(19) United States
(12) Patent Application Publication
(10) Pub. No.: US 2023/0080513 A1
(43) Pub. Date: Mar. 16, 2023

(54) COMPOSITIONS AND METHODS FOR COMBINATORIAL ASSEMBLY AND SCREENING FOR FUNCTIONAL SITE-DIRECTED NUCLEOTIDE MODIFIERS

(71) Applicant: Meristem SpA, Huechuraba (CL)

(72) Inventors: Bernardo POLLAK WILLIAMSON, Santiago (CL); Diego Ignacio LAGOS SUSAETA, Santiago (CL); Tomas Alberto NORAMBUENA ARENAS, Santiago (CL)

(73) Assignee: Meristem SpA, Santiago (CL)

(21) Appl. No.: 17/817,161

(22) Filed: Aug. 3, 2022

Related U.S. Application Data
(60) Provisional application No. 63/229,414, filed on Aug. 4, 2021.

(51) Int. Cl. C12N 15/10; C12N 9/78; C12N 9/22

(52) U.S. Cl. CPC C12N 15/102; C12N 9/78; C12N 9/22; C07K 2319/81; C12N 2310/20

(57) ABSTRACT

The present invention discloses a method where a site-directed nuclease (e.g. Cas nuclease) as well as a nucleotide modifying enzyme (e.g. deaminase) are provided as modules of a sequence assembly system, such as a golden gate cloning system, comprising cloning sites and/or regulatory sites. The assembly system provides capacities for generating a polypeptide consisting of at least two standardised modules, each which can be composed of multiple peptide motifs, which optionally can be joined along with other standardised modules for extending the polypeptide's functionality further. New assemblies intended for site-directed nucleotide modification are also provided in this invention.

### CROSS-REFERENCE TO RELATED APPLICATIONS

This application claims priority to U.S. Provisional Application No. 63/229,414, filed on Aug. 4, 2021, which is hereby incorporated by reference herein in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 230702000100SEQLIST.XML, date recorded: Aug. 2, 2022, size: 399,312 bytes).

### FIELD

This invention aims to provide methods and systems to assemble site-directed nucleases and nucleotide-modifying enzymes so as to develop nucleic acid modifiers in a multiple and combinatorial fashion.

The present invention is directed to develop molecular base-editors from systems relying on combinatorial methods combining different modules comprising site-directed nucleases, such as (but not limited to) Cas systems, and nucleotide modifying enzymes, such as (but not limited to) adenine and cytosine deaminases.

The invention described herein enables extension of functionality of the composed of base-editors by further composition with other fusion partners in a standardised manner. Peptide fusions can extend functionality of the polypeptide by conferring the polypeptide with the fusion partner's own molecular function.

### BACKGROUND

Site-directed nucleases such as Cas proteins, TALENs and Zinc finger nucleases enable sequence-specified enzymatic cleavage of nucleic acids. Engineering of these site-directed nucleases has enabled the development of nucleic acid binding proteins domains with high specificity which can be targeted to specific nucleic acid sequences (Jinek, M. et al., 2012, A programmable dual-RNA-guided DNA endonuclease in adaptive bacterial immunity. Science 337, 816-821). These domains, when fused to protein domains with nucleotide modification activity enable modification of nucleic acids.

For example, the combination of a Cas9 protein without cleavage activity and a cytosine deaminase domain enables the sequence-specified (in presence of a sgRNA complementary to a target sequence) modification of a cytosine to uracil nucleotide, which can lead to a thymine nucleotide replacement under certain conditions of DNA repair (Komor, A., Kim, Y., Packer, M. et al., 2016, Programmable editing of a target base in genomic DNA without double-stranded DNA cleavage. Nature 533, 420-424). This combination is commonly referred to as a base-editor.

Site-directed nucleases and DNA-modifying protein domains described to date are restricted to few classes (base-editors, targeted DNA-methylases and targeted DNA-demethylases, Cas-transposon systems) and only few functionally active combinations have been developed (Anzalone, A. V. etl al, 2020, Genome editing with CRISPR—Cas nucleases, base editors, transposases and prime editors. Nature biotechnology, 38(7), 824-844). However, there is an increasing number of individual protein modules (both site-directed nucleases and modifying protein modules) that are continuously being reported in the literature.

Methods for molecular recombination of nucleic acid sequences have been developed with the purpose of enhancing enzyme activity, protein expression or optimization of metabolic pathways but either require sequence homology for their success, or do not enable further flexibility for customisation and functionalisation. However, for the design and development of novel combinations of nucleic acid-modifying site-directed nucleases or their derivatives these methods are not adequate and novel nucleic acid-modifying site-directed nucleases are currently being developed through the ingenuity and expertise of skilled researchers. Standardised methods for the combinatorial composition, detection and functional characterization of nucleic-acid modifying enzymes, along with extension of function, have yet to be described.

### BRIEF SUMMARY

The present invention discloses a method where a site-directed nuclease as well as a nucleotide-modifying enzyme are provided as modules of a sequence assembly system, such as (but not limited to) a Golden Gate cloning system, comprising cloning sites and/or regulatory sites. In a variant of the present invention, the system comprises a plurality of site-directed nucleases as well as a plurality of nucleotide-modifying enzymes which are provided as modules of a sequence assembly system, comprising in turn a plurality of cloning sites and/or regulatory sites. The assembly strategy provides capacities for generating a polypeptide consisting of at least two standardised modules, each which can be composed of multiple peptide motifs, which optionally can be joined along with other standardised modules for extending the polypeptide's functionality further.

The following enumerated embodiments are representative of some aspects of the invention.
1. A method for obtaining assemblies of site-directed nucleases and nucleotide-modifying enzymes, the method comprising:
   a. providing a library of modular nucleic acid sequences comprising
      i. two or more site-directed nuclease modules each comprising a nucleic acid sequence encoding a different site-directed nuclease,
      ii. two or more nucleotide-modifying enzyme modules each comprising a nucleic acid sequence encoding a different nucleotide-modifying enzyme, and
      iii. optionally, one or more additional modules each comprising one or more nucleic acid sequences encoding other components;
   b. constructing two or more nucleic acid assemblies in vitro, wherein each nucleic acid assembly is constructed by joining a site-directed nuclease module with a nucleotide-modifying enzyme module to generate an nucleic acid assembly that encodes the site-directed nuclease and the nucleotide-modifying enzyme operably linked to express as a single polypeptide, and optionally, further joining one or more additional modules to the assembly nucleic acid sequence;
   c. expressing the two or more nucleic acid assemblies constructed in step (b) to produce the site-directed nucleases and the nucleotide-modifying enzymes as single polypeptides; and
   d. detecting a nucleotide-modifying activity for each of the single polypeptides expressed in step (c).
2. The method of embodiment 1, wherein the library was provided by:
   a-1 selecting two or more nucleic acid sequences each encoding a site-directed nuclease and two or more nucleic acid sequences each encoding a nucleotide-modifying enzyme;
   a-2 generating the two or more site-directed nuclease modules each comprising one of the two or more nucleic acid sequences encoding a site-directed nuclease selected in step (a-1); and
   a-3 generating the two or more nucleotide-modifying enzyme modules each comprising one of the two or more nucleic acid sequences encoding a nucleotide-modifying enzyme selected in step (a-1);
   a-4 optionally, generating one or more additional modules each comprising one or more nucleic acid sequences encoding other components; and
   a-5 optionally, codon optimizing one or more of the site-directed nuclease modules, the nucleotide modifying modules, the additional modules, or any combination thereof.
3. The method of embodiment 1 or embodiment 2, further comprising:
   e. analyzing each nucleotide-modifying activity detected to select one or more optimal nucleic acid assemblies.
4. The method of embodiment 3, further comprising:
   f. expressing the one or more optimal nucleic acid assemblies and purifying one or more polypeptides expressed from the optimal nucleic acid assembly.
5. The method of any one of embodiments 1 to 4, wherein the site directed nucleases comprise one or more Cas nucleases, one or more Cas nickases, one or more dead Cas nickases, or any combination thereof.
6. The method of any one of embodiments 1 to 5, wherein the site-directed nucleases are each independently selected from the group consisting of AsCas12a, Cas12a (Cpf1), Cas12b, Cas12e (CasX), Cas12j, Cas12f, Cas13a, Cas13b, Cas13d, Cas14a, Cas9, dCas9, Cas9-NGv1, CasX, CasY, CasΦ, CasΦ-2 (CasPhi2), dCasΦ-2 (dCasPhi2), CjCas9, eCas9, enAsCas12a, enAsCas12a-HF1, enLbCas12a, ErCas12a, eSpCas9, eSpCas9-1.1, FnCas12a, FnCas9, HF-nCAS9, HF2-CAS9, HypaCas9, LbCas12a, dLbCas12a, MbCas12a, NmCas9, SaCas9, SaurCas9, SauriCas9, ScCas9, SpCas9, SpCas9-HF1, SpCas9-NG, SpG, SpRY, SpyoCas9, St1Cas9, St3Cas9, StCas9, ttLbCas12a, xCas9, xCas9(3.6), and xCas9(3.7).
7. The method of any one of embodiments 1 to 5, wherein the site-directed nucleases comprise Cas12a (Cpf1), CasΦ, or a combination thereof.
8. The method of any one of embodiments 1 to 7, wherein the nucleotide-modifying enzymes comprise one or more deaminases, one or more demethylases, one or more methyltransferases, or any combination thereof.
9. The method of embodiment 8, wherein the nucleotide-modifying enzymes are deaminases.
10. The method of any one of embodiments 1 to 9, wherein the nucleotide-modifying enzymes are selected from the group consisting of APOBEC, APOBEC-like enzymes, AID, AID-like enzymes, A3A, DNMT3A, MQ3, LSD1, RrA3F, RrA3F-F130L, AmA1, SsA3B, SsA3B-R54Q, PpA1, PpA1-H122A, PpA1-R33A, Anc-rA1, rA1, AID, pmCDA1, A3A, eA3A, rA1-YE1, rA1-EE, rA1-YE2, rA1-YEE, A3A-Y130F, A3A-Y132D, A3B, AIDmono, TadA-7.10, CGBE1, hA3G-CTD, evoAPOBEC1, evoCDA1, evoFERNY, TadA-8e, TadA-9, minStart, and mTadA8e.
11. The method of any one of embodiments 1 to 10, wherein the library of modular nucleic acid sequences comprises one or more additional modules each comprising one or more nucleic acid sequences encoding other components.
12. The method of embodiment 11, wherein the additional modules comprise accessory component sequences encoding one or more accessory component polypeptides comprising a uracil-DNA glycosylase inhibitor (UGI), a fluorescent protein, a FokI protein, a methyltransferase, a Ψ synthase, a demethylase, one or more proteins of the plant abscisic acid (ABA) stress response pathway, a heterodimerizing leucine zipper, a Kruppel-associated Box (KRAB), a transcriptional activator, a repressive protein domain, an APOBEC enzyme, an APOBEC-like enzyme, an AID enzyme, an AID-like enzyme, or any combination thereof.
13. The method of embodiment 12, wherein the one or more accessory component polypeptides comprise a fluorescent protein.
14. The method of any one of embodiments 1 to 13, wherein one or more of the site-directed nuclease modules further comprise one or more linker sequences, each encoding a peptide linker and each operably linked to the nucleic acid sequence encoding a site-directed nuclease to express as a single polypeptide.
15. The method of any one of embodiments 1 to 14, wherein one or more of the nucleotide-modifying enzyme modules further comprise one or more linker sequences, each encoding a peptide linker and each operably linked to the nucleic acid sequence encoding a nucleotide-modifying enzyme to express as a single polypeptide.
16. The method of any one of embodiments 1 to 15, wherein one or more of the additional modules further comprise one or more linker sequences, each encoding a peptide linker and each operably linked to one or more of the accessory component sequences encoding one or more accessory component polypeptides to express as a single polypeptide.
17. The method of any one of embodiments 14 to 16, wherein the linker sequences encode peptide linkers comprising an XTEN peptide linker, a flexible linker GGS(xN), a polyproline motif, an all α-helical linker A(EAAAK)nA, or any combination thereof.
18. The method of any one of embodiments 1 to 17, wherein one or more of the site-directed nuclease modules further comprise one or more sequence motifs, each encoding a polypeptide motif and each operably linked to the nucleic acid sequence encoding a site-directed nuclease to express as a single polypeptide.
19. The method of any one of embodiments 1 to 18, wherein one or more of the nucleotide-modifying enzyme modules further comprise one or more sequence motifs, each encoding a polypeptide motif and each operably linked to the nucleic acid sequence encoding a nucleotide-modifying enzyme to express as a single polypeptide.
20. The method of any one of embodiments 1 to 19, wherein one or more of the additional modules further comprise one or more sequence motifs, each encoding a polypeptide motif and each operably linked to one or more of the accessory component sequences encoding one or more accessory component polypeptides to express as a single polypeptide.
21. The method of any one of embodiments 18 to 20, wherein the sequence motifs comprise a purification tag, a nuclear localization sequence, or a combination thereof.
22. The method of embodiment 21, wherein the purification tag is a 6×His peptide, optionally with a TEV protease cleavage site.
23. The method of embodiment 21, wherein the nuclear localization sequence is a SV40 nuclear localization sequence.
24. The method of any one of embodiments 1 to 23, wherein step (b) comprises constructing a combinatorial library of nucleic acid assemblies in vitro.
25. The method of any one of embodiments 1 to 24, wherein step (b) comprises constructing a plurality of nucleic acid assemblies in parallel.
26. The method of embodiment 25, wherein step (b) comprises constructing a plurality of nucleic acid assemblies in parallel in a single reaction.
27. The method of any one of embodiments 1 to 26, wherein step (b) comprises constructing two or more destination vectors, each comprising one of the nucleic acid assemblies.
28. The method embodiment 27, wherein constructing the two or more destination vectors comprises mixing two or more nucleic acid molecules each comprising one of the site-directed nuclease modules, one of the nucleotide-modifying enzyme modules, one of the additional modules, a protein-coding sequence, a regulatory sequence, or any combination thereof to produce the destination vectors.
29. The method of embodiment 28, wherein the nucleic acid molecules comprise one or more vectors, one or more linear polynucleotides, or a combination thereof.
30. The method of embodiment 29, wherein the vectors comprise one or more donor vectors each comprising one of the site-directed nuclease modules or one of the nucleotide-modifying enzyme modules.
31. The method of embodiment 29 or 30, wherein the vectors comprise one or more receiver vectors each comprising one of the site-directed nuclease modules or one of the nucleotide-modifying enzyme modules.
32. The method of any one of embodiments 28 to 31, wherein the protein-coding sequences comprise sequences encoding nucleotide-modifying enzymes, sequences encoding site-directed nucleases, sequences encoding linker peptides, sequences encoding polypeptide motifs, sequences encoding accessory component polypeptides, or any combination thereof.
33. The method of any one of embodiments 28 to 32, wherein the regulatory sequences comprise promoters, ribosome binding sites (RBSs), terminators, or any combination thereof.
34. The method of any one of embodiments 27 to 33, wherein constructing the two or more destination vectors comprises mixing the two or more nucleic acid molecules in a single reaction to produce the destination vectors.
35. The method of any one of embodiments 28 to 34, wherein the nucleic acid molecules comprise nucleic acid molecules that were synthesized in vitro.
36. The method any one of embodiments 27 to 35, wherein each destination vector further comprises a first promoter, a terminator, or a combination thereof, each operably linked to the nucleic acid assembly for expression of the nucleic acid assembly.
37. The method of embodiment 36, wherein the first promoter is a constitutive promoter.
38. The method of any one of embodiments 28 to 37, wherein nucleic acid molecules each comprise one or more Type IIS restriction sites such that digestion of the nucleic acid molecules with one or more corresponding Type IIS restriction enzymes produces linear polynucleotides with cohesive overhangs.
39. The method of embodiment 38, wherein the nucleic acid molecules comprise:
   i. a nucleic acid molecule comprising a promoter flanked by Type IIS restriction sites, wherein digestion of the nucleic acid molecule with one or more corresponding Type IIS restriction enzymes produces a linear polynucleotide comprising 5′ overhang A, the promoter, and 3′ overhang B;
   ii. a nucleic acid molecule comprising a 5′ untranslated region (UTR) and a ribosome binding site (RBS) flanked by Type IIS restriction sites, wherein digestion of the nucleic acid molecule with one or more corresponding Type IIS restriction enzymes produces a linear polynucleotide comprising 5′ overhang B, the 5′ UTR, the RBS, and 3′ overhang C;
   iii. a nucleic acid molecule comprising a nucleotide-modifying enzyme module flanked by Type IIS restriction sites, wherein digestion of the nucleic acid molecule with one or more corresponding Type IIS restriction enzymes produces a linear polynucleotide comprising 5′ overhang C, the nucleotide-modifying enzyme module, and 3′ overhang X;
   iv. a nucleic acid molecule comprising a site-directed nuclease module flanked by Type IIS restriction sites, wherein digestion of the nucleic acid molecule with one or more corresponding Type IIS restriction enzymes produces a linear polynucleotide comprising 5′ overhang X, the site-directed nuclease module, and 3′ overhang D;
   v. a nucleic acid molecule comprising one or more additional modules flanked by Type IIS restriction sites, wherein digestion of the nucleic acid molecule with one or more corresponding Type IIS restriction enzymes produces a linear polynucleotide comprising 5′ overhang D, the one or more additional modules, and 3′ overhang E; and/or
   vi. a nucleic acid molecule comprising a 3′ UTR and a terminator sequence flanked by Type IIS restriction sites, wherein digestion of the nucleic acid molecule with one or more corresponding Type IIS restriction enzymes produces a linear polynucleotide comprising 5′ overhang E, the site-directed nuclease module, and 3′ overhang F.
40. The method of embodiment 39, wherein overhang A is GGAG, overhang B is TACT, overhang C is AATG, overhang X is TGGA, overhang D is AGGT, overhang E is GCTT, and/or overhang F is CGCT.
41. The method of any one of embodiments 38 to 40, wherein the one or more corresponding Type IIS restriction enzymes comprise BsaI, SapI, or a combination thereof.
42. The method of any one of embodiments 27 to 41, wherein constructing the two or more destination vectors comprises mixing two or more nucleic acid molecules with the Type IIS restriction enzymes, a ligase enzyme, or a combination thereof in a single reaction mixture to produce the destination vectors.
43. The method of any one of embodiments 1 to 42, wherein step (c) comprises
   c-1 transforming two or more host cells with two or more destination vectors, and optionally with one or more additional vectors; and
   c-2 culturing the host cells and allowing expression of the nucleotide assembly to produce the site-directed nucleases and the nucleotide-modifying enzymes as single polypeptides.
44. The method of embodiment 43, wherein the host cells are yeast cells or bacterial cells.
45. The method of embodiment 43 or 44, wherein the destination vector further comprises a gRNA expression cassette comprising a gRNA sequence operably linked to a second promoter, a target-reporter expression cassette, or a combination thereof.
46. The method of any one of embodiments 43 to 45, wherein step (c-1) comprises transforming a host cell with one or more additional vectors.
47. The method of embodiment 46, wherein the one or more additional vectors comprise a gRNA expression cassette comprising a gRNA sequence operably linked to a second promoter, a target-reporter expression cassette comprising a target sequence and a reporter sequence operably linked to a third promoter, or a combination thereof.
48. The method of any one of embodiments 45 to 47, wherein the target-reporter expression cassette comprises a target sequence and a reporter sequence operably linked to a third promoter.
49. The method of embodiment 48, wherein the target sequence and the reporter sequence are operably linked to express as a single reporter polypeptide.
50. The method of embodiment 47 or 49, wherein the reporter sequence encodes a reporter molecule.
51. The method of embodiment 50, wherein the reporter molecule is a fluorescent protein.
52. The method of embodiment 51, wherein the fluorescent protein is GFP or sfGFP.
53. The method of any one of embodiments 47 to 52, wherein the third promoter is a constitutive promoter.
54. The method of any one of embodiments 47 to 53, wherein the target sequence comprises one or more target nucleotides for editing, wherein editing of the one or more target nucleotides results in the formation of an edited target sequence having one or more stop codons.
55. The method of embodiment 54, wherein editing of the one or more target nucleotides results in the expression of the reporter polypeptide.
56. The method of embodiment 54, wherein editing of the one or more target nucleotides results in the non-expression of the reporter polypeptide.
57. The method of any one of embodiments 43 to 56, wherein step (c) further comprises
   c-3 spreading the transformed host cells on a solid medium;
   c-4 allowing colonies to form on the solid medium; and
   c-4 allowing expression of the nucleotide assembly in the colonies to produce the site-directed nucleases and the nucleotide-modifying enzymes as single polypeptides.
58. The method of any one of embodiments 1 to 57, wherein step (d) comprises DNA sequencing, PCR, colony screening, detecting fluorescence, or any combination thereof.
59. The method any one of embodiments 49 to 58, wherein step (d) comprises detecting expression or non-expression of the reporter polypeptide in the host cells.
60. The method of embodiment 59, wherein the expression or non-expression of the reporter polypeptide is detected in liquid cultures.
61. The method of embodiment 59, wherein the expression or non-expression of the reporter polypeptide is detected in colonies on solid medium.
62. The method of embodiment 61, wherein step (d) further comprises identifying cells with non-expression of the reporter polypeptide as edited cells.
63. The method of embodiment 62, wherein each colony of edited cells is isolated and grown in a pure culture.
64. The method of any one of embodiments 3 to 63, wherein step (e) comprises sequencing the destination vectors, nucleic acid assemblies, edited target sequences, or any combination thereof of the edited cells.
65. The method of embodiment 64, wherein step (e) further comprises identifying the modified sites in each of the edited target sequences.
66. The method of embodiment 64 or 65, wherein step (e) further comprises determining the sequence context of the modified sites in each of the edited target sequences.
67. The method of any one of embodiments 64 to 66, wherein step (e) further comprises identifying the window of modification in each of the target sequences.
68. The method of any one of embodiments 3 to 67, wherein step (e) further comprises detecting off-target nucleotide edits in the edited cells by isolating and sequencing the DNA of the edited cells, wherein the DNA comprises destination vectors, genomic DNA, or a combination thereof.
69. The method of any one of embodiments 4 to 68, wherein step (f) comprises cloning the one or more optimal nucleic acid assemblies into an expression vector comprising the optimal nucleic acid assembly operably linked to a fourth promoter.
70. The method of embodiment 69, wherein the fourth promoter is a constitutive promoter.
71. The method of any one of embodiments 4 to 70, wherein step (f) comprises transforming cells with the expression vector, culturing the transformed cells, and allowing expression of one or more polypeptides encoded by the optimal nucleic acid assembly.
72. The method of embodiment 69, wherein the fourth promoter is an inducible promoter.
73. The method of embodiment 72, wherein step (f) comprises transforming cells with the expression vector, culturing the transformed cells, and inducing expression of one or more polypeptides encoded by the optimal nucleic acid assembly.
74. The method of any one of embodiments 4 to 73, further comprising purifying the one or more polypeptides encoded by the optimal nucleic acid assembly.
75. The method of embodiment 74, further comprising concentrating the one or more polypeptides encoded by the optimal nucleic acid assembly.
76. A method of selecting an optimal nucleic acid assembly for editing a target nucleotide, comprising the steps of any one of embodiments 1 to 75.
77. A library of nucleic acid assemblies of site-directed nucleases and nucleotide-modifying enzymes, comprising
   i. two or more site-directed nuclease modules each comprising a nucleic acid sequence encoding a different site-directed nuclease,
   ii. two or more nucleotide-modifying enzyme modules each comprising a nucleic acid sequence encoding a different nucleotide-modifying enzyme, and
   iii. optionally, one or more additional modules each comprising one or more nucleic acid sequences encoding other components;
   wherein each nucleic acid assembly comprises a site-directed nuclease module and a nucleotide-modifying enzyme module, wherein each nucleic acid assembly encodes the site-directed nuclease, the nucleotide-modifying enzyme, and, optionally, one or more additional modules, operably linked to express as a single polypeptide.
78. The library of embodiment 77, wherein the site directed nucleases comprise one or more Cas nucleases, one or more Cas nickases, one or more dead Cas nickases, or any combination thereof.
79. The library of embodiment 77 or 78, wherein the site-directed nucleases are each independently selected from the group consisting of AsCas12a, Cas12a (Cpf1), Cas12b, Cas12e (CasX), Cas12j, Cas12f, Cas13a, Cas13b, Cas13d, Cas14a, Cas9, dCas9, Cas9-NGv1, CasX, CasY, CasΦ, CasΦ-2 (CasPhi2), dCasΦ-2 (dCasPhi2), CjCas9, eCas9, enAsCas12a, enAsCas12a-HF1, enLbCas12a, ErCas12a, eSpCas9, eSpCas9-1.1, FnCas12a, FnCas9, HF-nCAS9, HF2-CAS9, HypaCas9, LbCas12a, dLbCas12a, MbCas12a, NmCas9, SaCas9, SaurCas9, SauriCas9, ScCas9, SpCas9, SpCas9-HF1, SpCas9-NG, SpG, SpRY, SpyoCas9, St1Cas9, St3Cas9, StCas9, ttLbCas12a, xCas9, xCas9(3.6), and xCas9(3.7).
80. The library of any one of embodiments 77 to 79, wherein the site-directed nucleases comprise Cas12a (Cpf1), CasΦ, or a combination thereof.
81. The library of any one of embodiments 77 to 80, wherein the nucleotide-modifying enzymes comprise one or more deaminases, one or more demethylases, one or more methyltransferases, or any combination thereof.
82. The library of embodiment 81, wherein the nucleotide-modifying enzymes are deaminases.
83. The library of any one of embodiments 77 to 82, wherein the nucleotide-modifying enzymes are selected from the group consisting of APOBEC, APOBEC-like enzymes, AID, AID-like enzymes, A3A, DNMT3A, MQ3, LSD1, RrA3F, RrA3F-F130L, AmA1, SsA3B, SsA3B-R54Q, PpA1, PpA1-H122A, PpA1-R33A, Anc-rA1, rA1, AID, pmCDA1, A3A, eA3A, rA1-YE1, rA1-EE, rA1-YE2, rA1-YEE, A3A-Y130F, A3A-Y132D, A3B, AIDmono, TadA-7.10, CGBE1, hA3G-CTD, evoAPOBEC1, evoCDA1, evoFERNY, TadA-8e, TadA-9, minStart, and mTadA8e.
84. The library of any one of embodiments 77 to 83, wherein the library of modular nucleic acid sequences comprises one or more additional modules each comprising one or more nucleic acid sequences encoding other components.
85. The library of embodiment 84, wherein the additional modules comprise accessory component sequences encoding one or more accessory component polypeptides comprising a uracil-DNA glycosylase inhibitor (UGI), a fluorescent protein, a FokI protein, a methyltransferase, a Ψ synthase, a demethylase, one or more proteins of the plant abscisic acid (ABA) stress response pathway, a heterodimerizing leucine zipper, a Kruppel-associated Box (KRAB), a transcriptional activator, a repressive protein domain, an APOBEC enzyme, an APOBEC-like enzyme, an AID enzyme, an AID-like enzyme, or any combination thereof.
86. The method of embodiment 85, wherein the one or more accessory component polypeptides comprise a fluorescent protein.
87. The library of any one of embodiments 77 to 86, wherein one or more of the site-directed nuclease modules further comprise one or more linker sequences, each encoding a peptide linker and each operably linked to the nucleic acid sequence encoding a site-directed nuclease to express as a single polypeptide.
88. The library of any one of embodiments 77 to 87, wherein one or more of the nucleotide-modifying enzyme modules further comprise one or more linker sequences, each encoding a peptide linker and each operably linked to the nucleic acid sequence encoding a nucleotide-modifying enzyme to express as a single polypeptide.
89. The library of any one of embodiments 77 to 88, wherein one or more of the additional modules further comprise one or more linker sequences, each encoding a peptide linker and each operably linked to one or more of the accessory component sequences encoding one or more accessory component polypeptides to express as a single polypeptide.
90. The library of embodiment 89, wherein the linker sequences encode peptide linkers comprising an XTEN peptide linker, a flexible linker GGS(xN), a polyproline motif, an all α-helical linker A(EAAAK)nA, or any combination thereof.
91. The library of any one of embodiments 77 to 90, wherein one or more of the site-directed nuclease modules further comprise one or more sequence motifs, each encoding a polypeptide motif and each operably linked to the nucleic acid sequence encoding a site-directed nuclease to express as a single polypeptide.
92. The library of any one of embodiments 77 to 91, wherein one or more of the nucleotide-modifying enzyme modules further comprise one or more sequence motifs, each encoding a polypeptide motif and each operably linked to the nucleic acid sequence encoding a nucleotide-modifying enzyme to express as a single polypeptide.
93. The library of any one of embodiments 77 to 92, wherein one or more of the additional modules further comprise one or more sequence motifs, each encoding a polypeptide motif and each operably linked to one or more of the accessory component sequences encoding one or more accessory component polypeptides to express as a single polypeptide.
94. The library of embodiment 93, wherein the sequence motifs comprise a purification tag, a nuclear localization sequence, or a combination thereof.
95. The library of embodiment 94, wherein the purification tag is a 6×His peptide, optionally with a TEV protease cleavage site.
96. The library of embodiment 94, wherein the nuclear localization sequence is a SV40 nuclear localization sequence.
97. The library any one of embodiments 77 to 96, wherein the nucleic acid assemblies comprise:
   i. a promoter flanked by 5′ overhang A and 3′ overhang B;
   ii. a 5′ untranslated region (UTR) and a ribosome binding site (RBS) flanked by 5′ overhang B and 3′ overhang C;
   iii. a nucleotide-modifying enzyme module flanked by 5′ overhang C and 3′ overhang X;
   iv. a site-directed nuclease module flanked by 5′ overhang X and 3′ overhang D;
   v. one or more additional modules flanked by 5′ overhang D and 3′ overhang E; and/or
   vi. a 3′ UTR and a terminator sequence flanked by 5′ overhang E and 3′ overhang F.
98. The library of embodiment 97, wherein overhang A is GGAG, overhang B is TACT, overhang C is AATG, overhang X is TGGA, overhang D is AGGT, overhang E is GCTT, and/or overhang F is CGCT
99. A base editor for editing a target nucleotide, wherein the base editor comprises
   i. a site-directed nuclease module comprising a site-directed nuclease selected from the group consisting of AsCas12a, Cas12a (Cpf1), Cas12b, Cas12e (CasX), Cas12j, Cas12f, Cas13a, Cas13b, Cas13d, Cas14a, Cas9, dCas9, Cas9-NGv1, CasX, CasY, CasΦ, CasΦ-2 (CasPhi2), dCasΦ-2 (dCasPhi2), CjCas9, eCas9, enAsCas12a, enAsCas12a-HF1, enLbCas12a, ErCas12a, eSpCas9, eSpCas9-1.1, FnCas12a, FnCas9, HF-nCAS9, HF2-CAS9, HypaCas9, LbCas12a, dLbCas12a, MbCas12a, NmCas9, SaCas9, SaurCas9, SauriCas9, ScCas9, SpCas9, SpCas9-HF1, SpCas9-NG, SpG, SpRY, SpyoCas9, St1Cas9, St3Cas9, StCas9, ttLbCas12a, xCas9, xCas9(3.6), and xCas9(3.7);
   ii. a nucleotide-modifying enzyme module comprising a nucleotide-modifying enzyme selected from the group consisting of APOBEC, APOBEC-like enzymes, AID, AID-like enzymes, A3A, DNMT3A, MQ3, LSD1, RrA3F, RrA3F-F130L, AmA1, SsA3B, SsA3B-R54Q, PpA1, PpA1-H122A, PpA1-R33A, Anc-rA1, rA1, AID, pmCDA1, A3A, eA3A, rA1-YE1, rA1-EE, rA1-YE2, rA1-YEE, A3A-Y130F, A3A-Y132D, A3B, AIDmono, TadA-7.10, CGBE1, hA3G-CTD, evoAPOBEC1, evoCDA1, evoFERNY, TadA-8e, TadA-9, minStart, and mTadA8e; and
   iii. optionally, one or more additional modules comprising one or more additional polypeptides;
   wherein the site-directed nuclease module, the nucleotide-modifying enzyme module, and, optionally, one or more additional modules, are fused as a single polypeptide.
100. The base editor of embodiment 99, wherein the site-directed nuclease module comprises a site-directed nuclease selected from the group consisting of dCas9, dLbCas12a (dCpf1), and CasΦ-2 (CasPhi2).
101. The base editor of embodiment 99 or embodiment 100, wherein the nucleotide-modifying enzyme module comprises a nucleotide-modifying enzyme selected from the group consisting of A3A, Anc-rA1, eA3A, evoCDA1, minStart, mTadA8e, rA1, rA1-YE1, A3A-Y130F, A3B, AID, AmA1, evoAPOBEC1, evoFERNY, hA3G-CTD, pmCDA1, PpA1-R33A, rA1-YEE, RrA3F-F130L, and SsA3B-R54Q.
102. A base editor for editing a target nucleotide comprising a site-directed nuclease module and a nucleotide-modifying enzyme module, wherein:
   (1) the site-directed nuclease module comprises dCas9 and the nucleotide-modifying enzyme module comprises A3A;
   (2) the site-directed nuclease module comprises dCas9 and the nucleotide-modifying enzyme module comprises Anc-rA1;
   (3) the site-directed nuclease module comprises dCas9 and the nucleotide-modifying enzyme module comprises eA3A;
   (4) the site-directed nuclease module comprises dCas9 and the nucleotide-modifying enzyme module comprises evoCDA1;
   (5) the site-directed nuclease module comprises dCas9 and the nucleotide-modifying enzyme module comprises minStart;
   (6) the site-directed nuclease module comprises dCas9 and the nucleotide-modifying enzyme module comprises mTadA8e;
   (7) the site-directed nuclease module comprises dCas9 and the nucleotide-modifying enzyme module comprises rA1;
   (8) the site-directed nuclease module comprises dCas9 and the nucleotide-modifying enzyme module comprises rA1-YE1;
   (9) the site-directed nuclease module comprises dCasΦ-2 (dCasPhi2) and the nucleotide-modifying enzyme module comprises A3A;
   (10) the site-directed nuclease module comprises dCasΦ-2 (dCasPhi2) and the nucleotide-modifying enzyme module comprises A3A-Y130F;
   (11) the site-directed nuclease module comprises dCasΦ-2 (dCasPhi2) and the nucleotide-modifying enzyme module comprises eA3A;
   (12) the site-directed nuclease module comprises dCasΦ-2 (dCasPhi2) and the nucleotide-modifying enzyme module comprises evoCDA1;
   (13) the site-directed nuclease module comprises dCasΦ-2 (dCasPhi2) and the nucleotide-modifying enzyme module comprises minStart;
   (14) the site-directed nuclease module comprises dCasΦ-2 (dCasPhi2) and the nucleotide-modifying enzyme module comprises mTadA8e;
   (15) the site-directed nuclease module comprises dCasΦ-2 (dCasPhi2) and the nucleotide-modifying enzyme module comprises rA1;
   (16) the site-directed nuclease module comprises dCasΦ-2 (dCasPhi2) and the nucleotide-modifying enzyme module comprises rA1-YE1;
   (17) the site-directed nuclease module comprises dLbCas12a and the nucleotide-modifying enzyme module comprises A3A;
   (18) the site-directed nuclease module comprises dLbCas12a and the nucleotide-modifying enzyme module comprises A3B;
   (19) the site-directed nuclease module comprises dLbCas12a and the nucleotide-modifying enzyme module comprises AID;
   (20) the site-directed nuclease module comprises dLbCas12a and the nucleotide-modifying enzyme module comprises AmA1;
   (21) the site-directed nuclease module comprises dLbCas12a and the nucleotide-modifying enzyme module comprises evoAPOBEC1;
   (22) the site-directed nuclease module comprises dLbCas12a and the nucleotide-modifying enzyme module comprises evoFERNY;
   (23) the site-directed nuclease module comprises dLbCas12a and the nucleotide-modifying enzyme module comprises hA3G-CTD;
   (24) the site-directed nuclease module comprises dLbCas12a and the nucleotide-modifying enzyme module comprises minStart;
   (25) the site-directed nuclease module comprises dLbCas12a and the nucleotide-modifying enzyme module comprises pmCDA1;
   (26) the site-directed nuclease module comprises dLbCas12a and the nucleotide-modifying enzyme module comprises PpA1-R33A;
   (27) the site-directed nuclease module comprises dLbCas12a and the nucleotide-modifying enzyme module comprises rA1-YEE;
   (28) the site-directed nuclease module comprises dLbCas12a and the nucleotide-modifying enzyme module comprises RrA3F-F130L; or
   (29) the site-directed nuclease module comprises dLbCas12a and the nucleotide-modifying enzyme module comprises SsA3B-R54Q.
103. The base editor of any one of embodiments 99 to 102, wherein the base editor comprises one or more additional modules comprising one or more additional polypeptides.
104. The base editor of embodiment 103, wherein the one or more additional polypeptides comprises a polypeptide for detecting the presence or the expression of the base editor in a cell or in tissue.
105. The base editor of embodiment 103 or embodiment 104, wherein the one or more additional polypeptides comprise a fluorescent protein or a reporter protein.
106. The base editor of embodiment 105, wherein the fluorescent protein is mNeonGreen (NG).
107. The base editor of any one of embodiments 103-106, wherein the one or more additional modules comprise one or more accessory component polypeptides comprising a uracil-DNA glycosylase inhibitor (UGI), a fluorescent protein, a FokI protein, a methyltransferase, a Ψ synthase, a demethylase, one or more proteins of the plant abscisic acid (ABA) stress response pathway, a heterodimerizing leucine zipper, a Kruppel-associated Box (KRAB), a transcriptional activator, a repressive protein domain, an APOBEC enzyme, an APOBEC-like enzyme, an AID enzyme, an AID-like enzyme, or any combination thereof.
108. The base editor of any one of embodiments 99 to 107, wherein the site-directed nuclease module further comprises one or more peptide linkers fused to the site-directed nuclease.
109. The base editor of any one of embodiments 99 to 108, wherein the nucleotide-modifying enzyme module further comprises one or more peptide linkers fused to the nucleotide-modifying enzyme.
110. The base editor of any one of embodiments 99 to 109, wherein the one or more additional modules further comprise one or more peptide linkers fused to the one or more additional polypeptides.
111. The base editor of any one of embodiments 108 to 110, wherein the one or more peptide linkers comprise an XTEN peptide linker, a flexible linker GGS(xN), a polyproline motif, an all α-helical linker A(EAAAK)nA, or any combination thereof.
112. The base editor of any one of embodiments 99 to 111, wherein the site-directed nuclease module further comprises one or more polypeptide motifs fused to the site-directed nuclease.
113. The base editor of any one of embodiments 99 to 112, wherein the nucleotide-modifying enzyme module further comprises one or more polypeptide motifs fused to the nucleotide-modifying enzyme.
114. The base editor of any one of embodiments 99 to 112, wherein the one or more additional modules further comprises one or more polypeptide motifs fused to the one or more additional polypeptides.
115. The base editor of any one of embodiments 112 to 114, wherein the polypeptide motifs comprise a purification tag, a nuclear localization sequence, or a combination thereof.
116. The base editor of embodiment 115, wherein the purification tag is a 6×His peptide, optionally with a TEV protease cleavage site.
117. The base editor of embodiment 115, wherein the nuclear localization sequence is a SV40 nuclear localization sequence.
118. A nucleic acid comprising a nucleic acid sequence encoding the base editor of any one of embodiments 99-117.
119. The nucleic acid of embodiment 118, further comprising a promoter operably linked to the nucleic acid sequence.
120. The nucleic acid of embodiment 118 or 119, wherein the nucleic acid sequence encoding the base editor has at least 75%, 80%, 85%, 90%, 91%, 92%, 93%, 94%, 94%, 95%, 96%, 97%, 98%, 99%, or 100% sequence identity to a nucleic acid sequence selected from the group consisting of SEQ ID NOs: 1-60.
121. A base editor for editing a target nucleotide, wherein the base editor has at least 75%, 80%, 85%, 90%, 91%, 92%, 93%, 94%, 94%, 95%, 96%, 97%, 98%, 99%, or 100% sequence identity to the amino acid sequence encoded by a nucleic acid sequence selected from the group consisting of SEQ ID NOs: 1-60.

### DESCRIPTION OF THE FIGURES

The present application can be understood by reference to the following description taken in conjunction with the accompanying figures.

FIG. 1. General depiction of the system here disclosed. The system comprises a number of steps which separates different subprocesses. These steps can be considered as part of a flowchart which comprises, but is not limited to, one or more of one through six logical steps, namely: selection of nucleases and nucleotide-modifying enzymes, e.g. deaminase (step 1); modular design and combinatorial assembly (step 2), construction of the assemblies (step 3), detection in vivo and isolation in vitro (step 4), characterisation of constructs (step 5); and optimised production of assemblies for use (step 6).

FIG. 2. Combinatorial assembly process as part of step 2 of the system here disclosed. Modular components representing LO elemental DNA parts including promoters, protein domains of particular function and terminator sequences are flanked by specific nucleotide sequences (herein defined by the symbols A, C, X, D, E, F) used for directional assembly using a Golden Gate-mediated cloning strategy. Specific overhang sequences used for directional assembly of LO parts are also used in order to establish functional designation of modules according to a gene model with determined ontology (promoter and 5′ untranslated region (UTR) with RBS, CDS segment 1, CDS segment 2, CDS segment 3,3′UTR and terminator). A detailed illustration of a nucleic acid-modifying domain (deaminase) and of a site-directed nuclease (Cas module) with constituting elements (6×His, NLS, XTEN, SGGS) is also shown.

FIG. 3. Main components for pBE vectors comprising: sfGFP editing target (for cytosine or adenine deaminase based BEs), gRNA (for Cas9 or Cas12 based BEs) and BE entry site (Top) or cloned BE (Bottom).

FIG. 4. Design of pre-sfGFP region in detail. Top: four putative stop codons that can be converted to stop codons by CBEs. Bottom: one stop codon that can be reversed to a nonstop stop codon by ABEs.

FIG. 5. Efficiency and prevalence of BE (deaminase-nuclease) in a combinatorial assembly experiment.

FIG. 6. Frequency of editing by CBEs on the cytidine of each four putative stop codons in the pre-sfGFP region.

FIGS. 7A-7B. Overview of combinatorial experiment. FIG. 7A shows the first half of a combinatorial experiment. FIG. 7B shows the second half of a combinatorial experiment.

FIGS. 8A-8C. Overall editing efficiency (all nucleotide positions tested on the pre-sfGFP sequence) of exemplary assembled base editors. FIG. 8A shows editing efficiency of base editors assembled in a combinatorial experiment with dCas9 and a list of deaminases. FIG. 8B shows editing efficiency of base editors assembled in a combinatorial experiment with dLbCas12a and a list of deaminases. FIG. 8C shows editing efficiency of base editors assembled in a combinatorial experiment with dCasPhi2 and a list of deaminases.

FIG. 9A-9C. Example of base editing windows where assembled base editors are tested. Efficiency is indicated as a percentage of single nucleotide editing at known positions on the pre-sfGFP sequence. Positions 1-4 represent the four putative stop codons within the pre-sfGFP sequence of the pBE9 and pBE12 plasmids that can be converted to stop codons when edited by a CBE. FIG. 9A shows editing windows of base editors assembled in a combinatorial experiment with dCas9 and a list of deaminases. FIG. 9B shows editing windows of base editors assembled in a combinatorial experiment with dLbCas12a and a list of deaminases. FIG. 9C shows editing windows of base editors assembled in a combinatorial experiment with dCasPhi2 and a list of deaminases.

FIGS. 10A-10B. Example of functionalization of base editors. In this case the base editor (A3A-dCas9) was assembled together with a fluorescent protein (NG) as a C-terminal part, and under the regulation of promoters p35S or pCmYLCV. In order to verify the expression as well as the localization of the base editor in a cell/tissue context, the corresponding vector containing the assembly was introduced into Nicotiana benthamiana tissue using Agrobacterium tumefaciens transformation. FIG. 10A shows expression of the NG-labeled A3A-dCas9 base editor under the control of the p35S promoter. FIG. 10B shows expression of the NG-labeled A3A-dCas9 base editor under the control of the pCmYLCV promoter.

### DETAILED DESCRIPTION

The following description of the embodiments is not intended to limit the invention to these embodiments, but rather to enable any person skilled in the art to make and use.

### Overview

This invention provides methods and systems to assemble site-directed nucleases and nucleotide-modifying enzymes to obtain nucleic acid modifiers in a multiple and combinatorial fashion. Furthermore, the invention provides new nucleic acid-modifiers obtained with such methodologies.

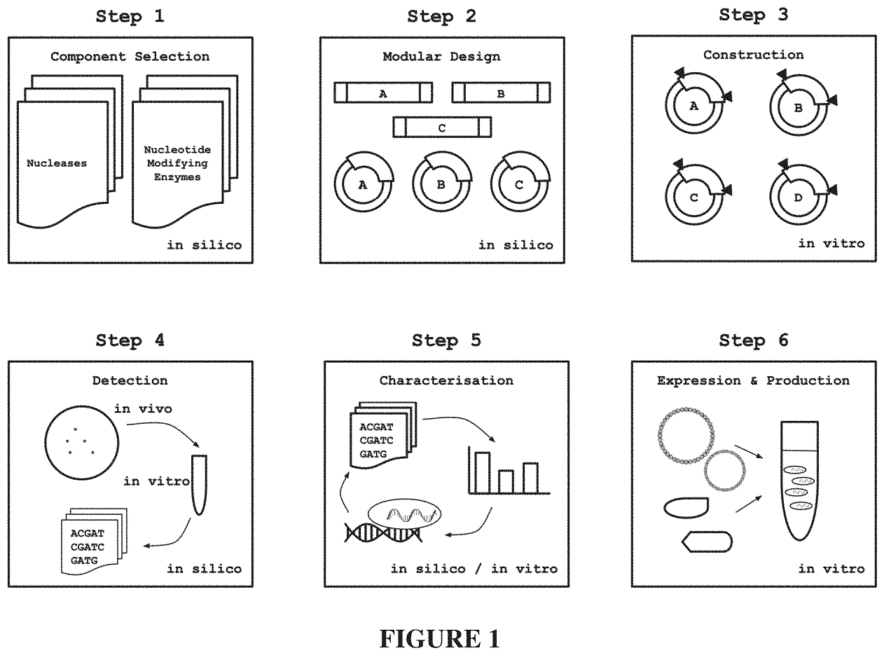

The system provided in this invention (FIG. 1) comprises a component selection step 1, which in turn comprises an in silico selected list of site-directed nucleases and a selected list of nucleotide-modifying enzymes. According to this invention the list of site-directed nucleases comprises, but is not limited to, Cas nucleases, as nickases or dead nickases. In the same way, amongst the nucleotide-modifying enzymes, the disclosed system can comprise any kind of deaminases, demethylases, methyltransferases, or any kind of enzyme able to modify nucleotides.

As part of the disclosed invention, the system comprises a combinatorial assembly step (step 2, in FIG. 1), which is designed in silico. This step comprises the modularisation of the different components found in a site-directed nuclease nucleotide-modifying enzyme assembly. The different components of such an assembly comprise, but are not limited to, a site-directed nuclease (selected from step 1 of the system), a nucleotide-modifying enzyme (selected also from step 1 of the system), and a linker (which links the nuclease and the nucleotide modifying enzyme). Other components can be present or not in the assemblies. These components comprise other accessory or modifying enzymes and/or event-triggering enzymes, as well as additional linkers and/or useful sequence motifs for post-processing events.

The step 3 of the system (FIG. 1) comprises the implementation of the steps described above experimentally, namely the construction of the assemblies in vitro. This step involves the combination of components bearers (e.g plasmids) in an in vitro reaction together with all other components allowing this reaction to occur, which comprise a proper medium and restriction endonucleases.

The step 4 (FIG. 1) involves the detection of the assemblies previously constructed. This process comprises the construction of libraries by cloning the assemblies as vectors into microorganisms able to keep such vectors and reproduce themself (e.g. bacterial cells). The detection can be achieved by incorporating a target sequence to be modified by the expressed assembly encoded in the vector. The modification of the target sequence accounts for a response which is measured or read by a detector or any device able to do so. This target sequence can be encoded in the same vector of the assembly or encoded in a different vector. The target sequence can be modified by guiding the assembly to a specific site inside the sequence. The final part of this step comprises the unveiling of the modified sequence by identifying the encoded nucleotide sequence and comparing it with the original target sequence.

A characterisation step (step 5, FIG. 1) can be also part of this disclosure, and comprises the determination of key features of the assemblies, according to the different parts composing them. This characterisation process involves the determination of an optimal modifying window that is the range at the target sequence which the assembly acts on, and the sequence context around which the modification can be accomplished e.g. seed/proximal or distal motifs. The step 5 of the disclosed system is optional since the characterisation process is not a mandatory step to provide new functional nuclease-modifying nucleotide enzyme assemblies, but it may be useful to provide information on specificity.

The final step (step 6, FIG. 1) comprises the expression, production and isolation in vitro of the assemblies previously constructed (and/or characterised). This step involves the combination of the required components and necessary media to accomplish such a production, and comprises the provision of necessary enzymes as well as one or more vectors encoding the assembly and/or the guide-sequence (e.g. guide-RNA) to direct the assembly to specific sequences to be modified. The assembly encoded in a vector certainly comprises at least an encoded nuclease and an encoded nucleotide-modifying enzyme, and may also comprise one or more encoded linkers and/or encoded accessory enzymes responsible for triggering other events associated with the nucleotide modification.

### Definitions

For the purposes and clarity of this disclosure some of the terms used in this invention must be defined.

The term nuclease is intended to define an enzyme of natural or artificial origin which binds one or more nucleotide sequences (e.g. DNA or RNA) and exerts (or not) one or multiple modifications on them. This nuclease can bind a nucleotide sequence through the recognition of a motif in the sequence (e.g an enzyme containing zinc-fingers which binds DNA at specific sites) or by assistance of another (specific) nucleotide sequence (e.g. Cas systems and related enzymes, which use RNA sequences as a guide to be directed to a specific site in the DNA).

A nucleotide-modifying enzyme typically defines an enzyme of natural or artificial origin that is able to modify the identity of nucleotides (ribonucleotides and/or deoxyribonucleotides) and/or attach or link other chemical moieties to them. The modification of nucleotides can be accomplished on a single or more free nucleotides, and in a sequence context as well.

The term linker typically refers to a chemical moiety that is attached (e.g. through covalent bonds) to two or more chemical parts originally isolated. A linker can be a polymer where each of its extremes bind covalently to major molecular complexes (e.g. a polypeptide which links two major proteins). The requirement of a chemical moiety for being a linker is to have a minimal or no effect on the properties and/or function of the parts it binds. Nonetheless, these chemical moieties can have a crucial role on the function of one of the major complexes, e.g. a nucleotide-modifying enzyme when linked to a nuclease may not be able to reach its target if the linker is not long enough.

In this invention, an accessory enzyme typically refers to an enzyme that can help or assist the process of nucleotide modification by interfering with the molecular events that promote such a modification or the events that prevent it. For example, if the modification of a nucleotide occurs in an in vivo context and there is a molecular machinery that can revert the modification, then it is desirable to have an accessory enzyme that impedes such a molecular machinery to act in order for the modification to linger.

A target sequence is typically a sequence of natural or artificial origin that contains the nucleotide(s) to be modified. This sequence must fulfill the requirements that allow a nuclease and a nucleotide-modifying enzyme to exert their function (e.g. binding and recognition requirements). This sequence can code a protein, can have a regulatory element, and/or any site of interest to be modified.

A guide nucleotide sequence is typically a sequence (e.g. RNA or DNA) that helps or assists a nuclease to recognise or to be directed to a specific site inside a target sequence. The guide nucleotide can be of natural or artificial origin and can be attached to the nuclease itself.

The term assembly is typically defined as a combination of elements that form a composite that has one or multiple properties and/or functions. In the context of this invention, an assembly refers to the combination of components encoded in a nucleotide sequence (e.g proteins, promoters, and/or regulatory sites encoded in a vector), and/or to the product encoded in such a nucleotide sequence (e.g. a number of linked proteins).

The term combinatorial assembly typically refers to the combination of elements in all possible configurations yielding, in turn, all possible composites or assemblies, taking into account the term assembly defined above.

The term reporter protein typically refers to a protein or enzyme that encompasses an observable or measurable characteristic in a cell or tissue context. Examples of reporter proteins include, e.g. a fluorescent protein, a luciferase enzyme, a beta-galactosidase enzyme, a (3-glucuronidase enzyme (e.g., encoded by a GUS reporter gene) and the like.

### DETAILED DESCRIPTION OF THE EMBODIMENTS

In a preferred embodiment, the system or method for obtaining assemblies of site-directed nucleases and nucleotide-modifying enzymes, comprises providing, in first step, a library of modular nucleic acid sequences composed of two or more site-directed nuclease modules each comprising a nucleic acid sequence encoding a different site-directed nuclease, two or more nucleotide-modifying enzyme modules each comprising a nucleic acid sequence encoding a different nucleotide-modifying enzyme, and optionally, one or more additional modules each comprising one or more nucleic acid sequences encoding other components. In a next step, the method comprises constructing two or more nucleic acid assemblies in vitro, wherein each nucleic acid assembly is constructed by joining a site-directed nuclease module with a nucleotide-modifying enzyme module to generate a nucleic acid assembly that encodes the site-directed nuclease and the nucleotide-modifying enzyme operably linked to express as a single polypeptide, and optionally, further joining one or more additional modules to the assembly nucleic acid sequence. In a further step, the method comprises expressing the two or more nucleic acid assemblies constructed in step in the previous step to produce the site-directed nucleases and the nucleotide-modifying enzymes as single polypeptides. In a final stage, the method comprises a step for detecting the nucleotide-modifying activity for each of the single polypeptides expressed in the previous step. Optionally, the method can comprise analyzing each nucleotide-modifying activity detected to select one or more optimal nucleic acid assemblies and/or expressing the one or more optimal nucleic acid assemblies and purifying one or more polypeptides expressed from the optimal nucleic acid assemblies. Additionally, the library aforementioned, is provided by selecting two or more nucleic acid sequences each encoding a site-directed nuclease and two or more nucleic acid sequences each encoding a nucleotide-modifying enzyme; by generating the two or more site-directed nuclease modules each comprising one of the two or more nucleic acid sequences encoding a site-directed nuclease selected previously; and by generating the two or more nucleotide-modifying enzyme modules each comprising one of the two or more nucleic acid sequences encoding a nucleotide-modifying enzyme selected previously. Optionally, the library can be provided by generating one or more additional modules each comprising one or more nucleic acid sequences encoding other components; and by codon optimizing one or more of the site-directed nuclease modules, the nucleotide modifying modules, the additional modules, or any combination thereof.

In a preferred embodiment, step 1 of the system here disclosed, comprises the selection of nucleases of the Cas type, which include, but are not limited to, AsCas12a, Cas12a (Cpf1), Cas12b, Cas12e (CasX), Cas12j, Cas12f, Cas13a, Cas13b, Cas13d, Cas14a, Cas9, dCas9, Cas9-NGv1, CasX, CasY, CasΦ, CasΦ-2 (CasPhi2), dCasΦ-2 (dCasPhi2), CjCas9, eCas9, enAsCas12a, enAsCas12a-HF1, enLbCas12a, ErCas12a, eSpCas9, eSpCas9-1.1, FnCas12a, FnCas9, HF-nCAS9, HF2-CAS9, HypaCas9, LbCas12a, dLbCas12a, MbCas12a, NmCas9, SaCas9, SaurCas9, SauriCas9, ScCas9, SpCas9, SpCas9-HF1, SpCas9-NG, SpG, SpRY, SpyoCas9, St1Cas9, St3Cas9, StCas9, ttLbCas12a, xCas9, xCas9(3.6), xCas9(3.7), and their corresponding nickase and dead versions. In the same way, amongst the nucleotide modifying enzymes, the disclosed system can comprise: APOBEC, APOBEC-like enzymes, AID, AID-like enzymes, A3A, DNMT3A, MQ3, LSD1, RrA3F, RrA3F-F130L, AmA1, SsA3B, SsA3B-R54Q, PpA1, PpA1-H122A, PpA1-R33A, Anc-rA1, rA1, AID, pmCDA1, A3A, eA3A, rA1-YE1, rA1-EE, rA1-YE2, rA1-YEE, A3A-Y130F, A3A-Y132D, A3B, AIDmono, TadA-7.10, CGBE1, hA3G-CTD, evoAPOBEC1, evoCDA1, evoFERNY, TadA-8e, TadA-9, minStart, and mTadA8e.

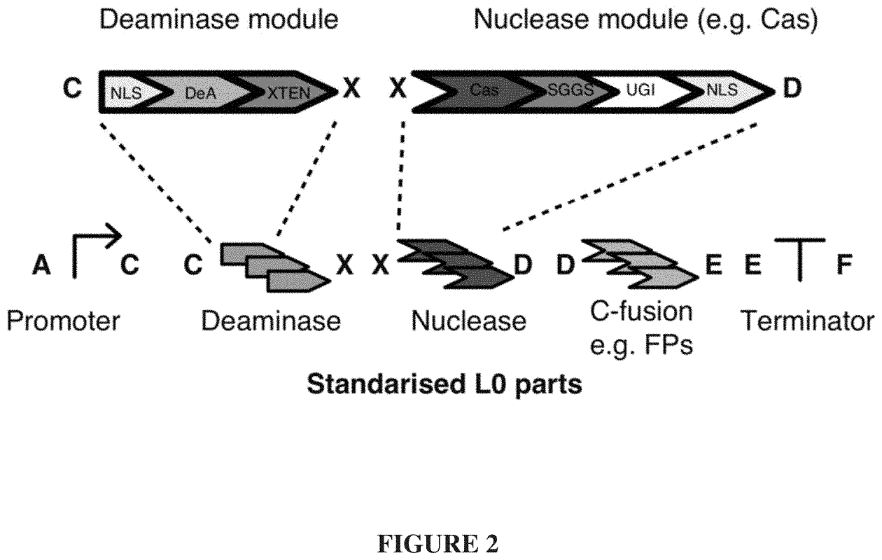

Embodiments of the invention disclosed herein, comprises the modularisation of the different components found in a nuclease-nucleotide modifying enzyme assembly through a combinatorial assembly step (step 2, FIG. 2). The different components of such an assembly may comprise, but are not limited to, a site-directed nuclease, a nucleotide-modifying enzyme (both described above), and a linker (which links the site-directed nuclease and the nucleotide-modifying enzyme). Thus, one or more of the site-directed nuclease modules may further comprise one or more linker sequences, each encoding a peptide linker and each operably linked to the nucleic acid sequence encoding a site-directed nuclease to express as a single polypeptide. The assembly can comprise further accessory or modifying enzymes and/or event-triggering enzymes, as well as additional linkers and/or useful sequence motifs for post-processing events. Amongst the other modifying enzymes and/or event-triggering enzymes, this invention comprises: uracil-DNA glycosylase inhibitors (UGI), fluorescent proteins, FokI, methyltransferases (e.g. m5U (like TrmA), 2′-O (like SpoU), DNMT3A or MQ3), Ψ synthases (like TruA), demethylases (e.g. LSD1), proteins of the plant abscisic acid (ABA) stress response pathway (e.g. PYL1 or ABI1), heterodimerising leucine zippers, Kruppel-associated Box (KRAB), transcriptional activators (e.g. VP64, P65, Rta, P65-HSF1 transactivation complex or MCP-fused VP64), repressive domains (e.g. SRDX), additional APOBEC or APOBEC-like enzymes, additional AID or AID-like enzymes. Additional modules may further comprise one or more sequence motifs, each encoding a polypeptide motif and each operably linked to one or more of the accessory component sequences encoding one or more accessory component polypeptides to express as a single polypeptide, wherein the sequence motifs comprise a purification tag, a nuclear localization sequence, or a combination thereof. The additional linkers and/or sequence motifs can be selected from: 6×His purification tag with TEV protease cleavage site, a SV40 nuclear localisation sequence, a XTEN peptide linker (US2011312881A1), a flexible linker such as a GGS(xN) repetition, more rigid linkers including polyproline motifs and an all α-helical linker A(EAAAK)nA; a nucleoplasmin nuclear localisation sequence; or any combination thereof.

In another embodiment of the invention, the method in the second step comprises constructing a combinatorial library of nucleic acid assemblies in vitro; constructing a plurality of nucleic acid assemblies in parallel; constructing a plurality of nucleic acid assemblies in parallel in a single reaction; and/or constructing two or more destination vectors, each comprising one of the nucleic acid assemblies. Regarding the latter, constructing the two or more destination vectors comprises, in turn, mixing two or more nucleic acid molecules each comprising one of the site-directed nuclease modules, one of the nucleotide-modifying enzyme modules, one of the additional modules, a protein-coding sequence, a regulatory sequence, or any combination thereof to produce the destination vectors. In this case, the nucleic acid molecules comprise one or more vectors, one or more linear polynucleotides, or a combination thereof, wherein the vectors may comprise one or more donor vectors each comprising one of the site-directed nuclease modules or one of the nucleotide-modifying enzyme modules, and wherein the vectors may comprise one or more receiver vectors each comprising one of the site-directed nuclease modules or one of the nucleotide-modifying enzyme modules. For its part, the protein-coding sequences may comprise sequences encoding nucleotide-modifying enzymes, sequences encoding site-directed nucleases, sequences encoding linker peptides, sequences encoding polypeptide motifs, sequences encoding accessory component polypeptides, or any combination thereof; and the regulatory sequences comprise promoters, ribosome binding sites (RBSs), terminators, or any combination thereof.

In addition, constructing the two or more destination vectors involves mixing the two or more nucleic acid molecules in a single reaction to produce the destination vectors, wherein the nucleic acid molecules comprise nucleic acid molecules that were synthesized in vitro. In some embodiments, each destination vector further comprises a first promoter, a terminator, or a combination thereof, each operably linked to the nucleic acid assembly for expression of the nucleic acid assembly. In this case, the first promoter can be a constitutive promoter.

Additionally or alternatively, the nucleic acid molecules each can comprise one or more Type IIS restriction sites such that digestion of the nucleic acid molecules with one or more corresponding Type IIS restriction enzymes produces linear polynucleotides with cohesive overhangs, which makes possible a combinatorial assembly step involving the digestion by a Type IIS restriction enzyme and consequent molecular ligation between compatible cohesive overhangs through the action of a DNA ligase. Multiple modules harbouring compatible overhangs can participate in the one-pot reaction to generate all possible combinations with cognate partners specified through the overhangs. Therefore, if added in equimolar concentrations, all possible combinations will be produced according to a probabilistic distribution. This method does not rely on sequence homology, but only on the compatibility of specified overhangs and enables defining the precise boundaries of the combinatorial assembly, as in the Golden Gate Shuffling method (Engler, C. et al., 2009, Golden Gate Shuffling: A One-Pot DNA Shuffling Method Based on Type IIs Restriction Enzymes. PLoS ONE 4(5), e5553). Thus the nucleic acid molecules can comprise a promoter flanked by Type IIS restriction sites, wherein digestion of the nucleic acid molecule with one or more corresponding Type IIS restriction enzymes produces a linear polynucleotide comprising 5′ overhang A, the promoter, and 3′ overhang B; a 5′ untranslated region (UTR) and a ribosome binding site (RBS) flanked by Type IIS restriction sites, wherein digestion of the nucleic acid molecule with one or more corresponding Type IIS restriction enzymes produces a linear polynucleotide comprising 5′ overhang B, the 5′ UTR, the RBS, and 3′ overhang C; a nucleotide-modifying enzyme module flanked by Type IIS restriction sites, wherein digestion of the nucleic acid molecule with one or more corresponding Type IIS restriction enzymes produces a linear polynucleotide comprising 5′ overhang C, the nucleotide-modifying enzyme module, and 3′ overhang X; a site-directed nuclease module flanked by Type IIS restriction sites, wherein digestion of the nucleic acid molecule with one or more corresponding Type IIS restriction enzymes produces a linear polynucleotide comprising 5′ overhang X, the site-directed nuclease module, and 3′ overhang D; one or more additional modules flanked by Type IIS restriction sites, wherein digestion of the nucleic acid molecule with one or more corresponding Type IIS restriction enzymes produces a linear polynucleotide comprising 5′ overhang D, the one or more additional modules, and 3′ overhang E; and/or a 3′ UTR and a terminator sequence flanked by Type IIS restriction sites, wherein digestion of the nucleic acid molecule with one or more corresponding Type IIS restriction enzymes produces a linear polynucleotide comprising 5′ overhang E, the site-directed nuclease module, and 3′ overhang F. Namely, in some embodiments, the overhang A is GGAG, overhang B is TACT, overhang C is AATG, overhang X is TGGA, overhang D is AGGT, overhang E is GCTT, and/or overhang F is CGCT. Additionally, these overhangs can be the results of the action of one or more Type IIS restriction enzymes, which comprise BsaI, SapI, or a combination thereof.

In the preferred embodiment, the step 3 of the system comprises the implementation of the steps described above experimentally, namely the synthesis of the different parts in order to construct the assemblies in vitro. This step involves the combination of synthesised vectors containing suitable restriction flanking sites for a proper cloning of nucleases and nucleotide-modifying enzymes. An in vitro reaction together with all other components (Type IIS restriction enzymes, a ligase enzyme, or a combination thereof) and a proper medium, allows the ligation to occur.

Additionally or alternatively, the step 4 of the method disclosed herein, comprises the construction of libraries by cloning the assemblies as vectors into microorganisms able to keep such vectors and reproduce themself (e.g. bacterial cells). The detection is achieved by means of a strategy which incorporates a target sequence to be modified by the expressed assembly encoded in the vector. This target sequence comprises, but is not limited to, a fluorescent protein or a reporter enzyme, which can be encoded in the same vector of the assembly or encoded in a different vector. The target sequence is modified by guiding the assembly to a specific site inside the sequence. To do so, the assembly comprises specific nucleotide sequences attached so as to be directed to the target to be modified (e.g. guide-sequence, guide-RNA). The final part of this step comprises the sequencing of the modified sequence by any means or technology available to do so, comprising Sanger sequencing, capillary electrophoresis, next-generation sequencing (NGS) such as Illumina or third-generation sequencing (TGS), including technologies such as Oxford Nanopore Technology (ONT), Pacific Biosciences (PB) or DNBSeq Technology (BGI).

In a variant of the embodiment, a characterisation step 5 can be performed. This step involves the determination of on-targets, off-targets, optimal editing window (position and scope), seed/proximal or distal motifs (e.g. sequence next to or distant from protospacer adjacent motifs in cases where nucleases require them), substrate preference and processivity, which are main parameters that define the performance of a nuclease-nucleotide modifying enzyme assembly.

In the preferred embodiment, the final step comprises the expression, production and isolation in vitro of the assemblies previously constructed (and/or characterised). This is performed by provisioning necessary enzymes as well as one or more vectors encoding the assembly and/or the guide-sequence (e.g. gRNA) to direct the assembly to specific sequences to be modified. The assembly encoded in a vector certainly comprises at least an encoded nuclease and an encoded nucleotide-modifying enzyme, and may also comprise one or more encoded linkers and/or encoded accessory enzymes responsible for triggering other events associated with the nucleotide modification. In this case, protein expression can be induced in a medium, and then total protein can be extracted, purified, concentrated and stored for later use.

In another embodiment of the invention, step 4 comprises transforming two or more host cells with two or more destination vectors, and optionally with one or more additional vectors; and culturing the host cells and allowing expression of the nucleotide assembly to produce the site-directed nucleases and the nucleotide-modifying enzymes as single polypeptides. In this case, the host cells can be yeast cells or bacterial cells. In turn, in some embodiments, the destination vector further comprises a gRNA expression cassette comprising a gRNA sequence operably linked to a second promoter, a target-reporter expression cassette, or a combination thereof. Such a target-reporter expression cassette can comprise a target sequence and a reporter sequence operably linked to a third promoter, or a combination thereof, and the target sequence and the reporter sequence can be operably linked to express as a single reporter polypeptide. The reporter sequence can encode a reporter molecule, which can be a fluorescent protein, or more specifically GFP, sfGFP, or mNeonGreen (NG). In turn, the third promoter can be a constitutive promoter, and the target sequence can comprise one or more target nucleotides for editing, wherein editing of the one or more target nucleotides results in the formation of an edited target sequence having one or more stop codons. Thus, editing of the one or more target nucleotides can result in the expression of the reporter polypeptide or in the non-expression of the reporter polypeptide.

Additionally or alternatively, step 4 of the methodology disclosed herein can comprise spreading the transformed host cells on a solid medium; allowing colonies to form on the solid medium; and allowing expression of the nucleotide assembly in the colonies to produce the site-directed nucleases and the nucleotide-modifying enzymes as single polypeptides.

In another embodiment of this disclosure, the final step can comprise DNA sequencing, PCR, colony screening, detecting fluorescence, or any combination thereof, as well as detecting expression or non-expression of the reporter polypeptide in the host cells, which can be detected in liquid cultures, or in colonies on solid medium. The step can also involve the identification of cells with non-expression of the reporter polypeptide as edited cells, wherein each colony of edited cells is isolated and grown in a pure culture. Furthermore, the step can comprise sequencing the destination vectors, nucleic acid assemblies, edited target sequences, or any combination thereof of the edited cells; identifying the modified sites in each of the edited target sequences; determining the sequence context of the modified sites in each of the edited target sequences; identifying the window of modification in each of the target sequences; detecting off-target nucleotide edits in the edited cells by isolating and sequencing the DNA of the edited cells, wherein the DNA comprises destination vectors, genomic DNA, or a combination thereof; cloning the one or more optimal nucleic acid assemblies into an expression vector comprising the optimal nucleic acid assembly operably linked to a fourth promoter, wherein the fourth promoter can be a constitutive promoter or an inducible promoter; transforming cells with the expression vector, culturing the transformed cells, and allowing expression of one or more polypeptides encoded by the optimal nucleic acid assembly; transforming cells with the expression vector, culturing the transformed cells, and inducing expression of one or more polypeptides encoded by the optimal nucleic acid assembly; purifying the one or more polypeptides encoded by the optimal nucleic acid assembly; and/or finally concentrating the one or more polypeptides encoded by the optimal nucleic acid assembly.

As part of this invention, the methodology and components described above are further useful for selecting an optimal nucleic acid assembly for editing a target nucleotide.

Also, as part of this invention, a library of nucleic acid assemblies of site-directed nucleases and nucleotide-modifying enzymes is provided. This library comprises two or more site-directed nuclease modules each comprising a nucleic acid sequence encoding a different site-directed nuclease; two or more nucleotide-modifying enzyme modules each comprising a nucleic acid sequence encoding a different nucleotide-modifying enzyme; and optionally, one or more additional modules each comprising one or more nucleic acid sequences encoding other components. In this case, each nucleic acid assembly comprises a site-directed nuclease module and a nucleotide-modifying enzyme module, wherein each nucleic acid assembly encodes the site-directed nuclease, the nucleotide-modifying enzyme, and, optionally, one or more additional modules, operably linked to express as a single polypeptide. In this library, the site directed nucleases may comprise one or more Cas nucleases, one or more Cas nickases, one or more dead Cas nickases, or any combination thereof, which can be each independently selected from the group consisting of AsCas12a, Cas12a (Cpf1), Cas12b, Cas12e (CasX), Cas12j, Cas12f, Cas13a, Cas13b, Cas13d, Cas14a, Cas9, dCas9, Cas9-NGv1, CasX, CasY, CasΦ, CasΦ-2 (CasPhi2), dCasΦ-2 (dCasPhi2), CjCas9, eCas9, enAsCas12a, enAsCas12a-HF1, enLbCas12a, ErCas12a, eSpCas9, eSpCas9-1.1, FnCas12a, FnCas9, HF-nCAS9, HF2-CAS9, HypaCas9, LbCas12a, dLbCas12a, MbCas12a, NmCas9, SaCas9, SaurCas9, SauriCas9, ScCas9, SpCas9, SpCas9-HF1, SpCas9-NG, SpG, SpRY, SpyoCas9, St1Cas9, St3Cas9, StCas9, ttLbCas12a, xCas9, xCas9(3.6), and xCas9(3.7). For its part, the nucleotide-modifying enzymes may comprise one or more deaminases, one or more demethylases, one or more methyltransferases, or any combination thereof, which can be selected from the group consisting of APOBEC, APOBEC-like enzymes, AID, AID-like enzymes, A3A, DNMT3A, MQ3, LSD1, RrA3F, RrA3F-F130L, AmA1, SsA3B, SsA3B-R54Q, PpA1, PpA1-H122A, PpA1-R33A, Anc-rA1, rA1, AID, pmCDA1, A3A, eA3A, rA1-YE1, rA1-EE, rA1-YE2, rA1-YEE, A3A-Y130F, A3A-Y132D, A3B, AIDmono, TadA-7.10, CGBE1, hA3G-CTD, evoAPOBEC1, evoCDA1, evoFERNY, TadA-8e, TadA-9, minStart, and mTadA8e.

The library so disclosed, can be composed of modular nucleic acid sequences which comprise one or more additional modules each comprising, in turn, one or more nucleic acid sequences encoding other components. The additional modules may comprise accessory component sequences encoding one or more accessory component polypeptides comprising a uracil-DNA glycosylase inhibitor (UGI), a fluorescent protein, a FokI protein, a methyltransferase, a Ψ synthase, a demethylase, one or more proteins of the plant abscisic acid (ABA) stress response pathway, a heterodimerizing leucine zipper, a Kruppel-associated Box (KRAB), a transcriptional activator, a repressive protein domain, an APOBEC enzyme, an APOBEC-like enzyme, an AID enzyme, an AID-like enzyme, or any combination thereof. In addition, the one or more of the site-directed nuclease modules may further comprise one or more linker sequences, each encoding a peptide linker and each operably linked to the nucleic acid sequence encoding a site-directed nuclease to express as a single polypeptide. The linker sequences can encode peptide linkers comprising an XTEN peptide linker, a flexible linker GGS(xN), a polyproline motif, an all α-helical linker A(EAAAK)nA, or any combination thereof. The site-directed nuclease modules can further comprise one or more sequence motifs, each encoding a polypeptide motif and each operably linked to the nucleic acid sequence encoding a site-directed nuclease to express as a single polypeptide. The sequence motifs can comprise a purification tag, a nuclear localization sequence, or a combination thereof, wherein the purification tag can be a 6×His peptide, optionally with a TEV protease cleavage site, and the nuclear localization sequence can be a SV40 nuclear localization sequence.

The nucleic acid assemblies of the library disclosed herein, can also comprise a promoter flanked by 5′ overhang A and 3′ overhang B; a 5′ untranslated region (UTR) and a ribosome binding site (RBS) flanked by 5′ overhang B and 3′ overhang C; a nucleotide-modifying enzyme module flanked by 5′ overhang C and 3′ overhang X; a site-directed nuclease module flanked by 5′ overhang X and 3′ overhang D; one or more additional modules flanked by 5′ overhang D and 3′ overhang E; and/or a 3′ UTR and a terminator sequence flanked by 5′ overhang E and 3′ overhang F. The overhangs can be the sites left by one or more Type IIS restriction enzymes. In this case, overhang A can be GGAG, overhang B can be TACT, overhang C can be AATG, overhang X can be TGGA, overhang D can be AGGT, overhang E can be GCTT, and/or overhang F can be CGCT.

As part of this invention, the methodology and components described above are further useful for selecting an optimal base editor for editing a target nucleotide.

As part of this invention, a base editor for editing a target nucleotide is provided. In some embodiments, the base editor comprises a site-directed nuclease module comprising a site-directed nuclease, a nucleotide-modifying enzyme module comprising a nucleotide-modifying enzyme, and, optionally, one or more additional modules comprising one or more additional polypeptides, wherein the site-directed nuclease module, the nucleotide-modifying enzyme module, and, optionally, one or more additional modules, are fused as a single polypeptide. In some embodiments, the a site-directed nuclease is selected from the group consisting of AsCas12a, Cas12a (Cpf1), Cas12b, Cas12e (CasX), Cas12j, Cas12f, Cas13a, Cas13b, Cas13d, Cas14a, Cas9, dCas9, Cas9-NGv1, CasX, CasY, CasΦ, CasΦ-2 (CasPhi2), dCasΦ-2 (dCasPhi2), CjCas9, eCas9, enAsCas12a, enAsCas12a-HF1, enLbCas12a, ErCas12a, eSpCas9, eSpCas9-1.1, FnCas12a, FnCas9, HF-nCAS9, HF2-CAS9, HypaCas9, LbCas12a, dLbCas12a, MbCas12a, NmCas9, SaCas9, SaurCas9, SauriCas9, ScCas9, SpCas9, SpCas9-HF1, SpCas9-NG, SpG, SpRY, SpyoCas9, St1Cas9, St3Cas9, StCas9, ttLbCas12a, xCas9, xCas9(3.6), and xCas9(3.7). In some embodiments, the nucleotide-modifying enzyme is selected from the group consisting of APOBEC, APOBEC-like enzymes, AID, AID-like enzymes, A3A, DNMT3A, MQ3, LSD1, RrA3F, RrA3F-F130L, AmA1, SsA3B, SsA3B-R54Q, PpA1, PpA1-H122A, PpA1-R33A, Anc-rA1, rA1, AID, pmCDA1, A3A, eA3A, rA1-YE1, rA1-EE, rA1-YE2, rA1-YEE, A3A-Y130F, A3A-Y132D, A3B, AIDmono, TadA-7.10, CGBE1, hA3G-CTD, evoAPOBEC1, evoCDA1, evoFERNY, TadA-8e, TadA-9, minStart, and mTadA8e.

The base editor so disclosed may comprise one or more additional modules comprising one or more additional polypeptides. The additional modules may comprise a polypeptide for detecting the presence or the expression of the base editor in a cell or in tissue. In some embodiments, the one or more additional polypeptides comprise a fluorescent protein (e.g., mNeonGreen (NG)) or a reporter protein (e.g., a reporter enzyme such as a luciferase enzyme, a beta-galactosidase enzyme, a β-glucuronidase enzyme (e.g., encoded by a GUS reporter gene)). The additional modules may comprise accessory component sequences encoding one or more accessory component polypeptides comprising a uracil-DNA glycosylase inhibitor (UGI), a fluorescent protein, a FokI protein, a methyltransferase, a Ψ synthase, a demethylase, one or more proteins of the plant abscisic acid (ABA) stress response pathway, a heterodimerizing leucine zipper, a Kruppel-associated Box (KRAB), a transcriptional activator, a repressive protein domain, an APOBEC enzyme, an APOBEC-like enzyme, an AID enzyme, an AID-like enzyme, or any combination thereof. In addition, the site-directed nuclease modules, nucleotide-modifying enzyme modules, and/or additional modules may further comprise one or more peptide linkers fused to the site-directed nuclease, the nucleotide-modifying enzyme, and/or the additional polypeptide. The peptide linkers may comprise an XTEN peptide linker, a flexible linker GGS(xN), a polyproline motif, an all α-helical linker A(EAAAK)nA, or any combination thereof. The site-directed nuclease modules, nucleotide-modifying enzyme modules, and/or additional modules can further comprise one or more sequence motifs, each encoding a polypeptide motif fused to the site-directed nuclease, the nucleotide-modifying enzyme, and/or the additional polypeptide. The sequence motifs can comprise a purification tag, a nuclear localization sequence, or a combination thereof, wherein the purification tag can be a 6×His peptide, optionally with a TEV protease cleavage site, and the nuclear localization sequence can be a SV40 nuclear localization sequence.

Sequences Encoding Site-Directed Nucleases, Nucleotide-Modifying Enzymes, and Accessory Components

TABLE 1
Nucleic acid sequences encoding site-directed nucleases, nucleotide-modifying
enzymes, and accessory components.

| SEQ | Category | Name | Nucleotide sequence |
|---|---|---|---|
| 1 | Base editor | A3A-dCas9 | atgggaagtcatcaccaccaccaccattcaggtgagaacctgtacttcca |
| | | | gagcatggctccgaaaaagaaacgtaaggtgggtatccacggcgttccgg |
| | | | cgatggaagcaagtccagcttcaggacccaggcacctgatggacccgcat |
| | | | attttcacctctaattttaacaacggtattggccgtcacaagacgtacct |
| | | | gtgttatgaagtggaacgtcttgacaacggcacctccgtgaaaatggacc |
| | | | agcatcgtggtttcttgcataatcaagctaaaaacctgctgtgcggtttc |
| | | | tacggccgtcacgcggaactgcgttttttggacctggttccgagcctgca |
| | | | actggacccggcacagatttatcgtgttacttggttcatcagctggtcgc |
| | | | cgtgttttagctggggttgcgccggcgaggtacgcgcgtttttgcaagag |
| | | | aacacccacgtgcgtttgcgcatcttcgccgctcgtatctacgattatga |
| | | | cccactgtataaagaagcgctccagatgctgcgcgatgcaggcgcgcagg |
| | | | tttccatcatgacctacgacgagttcaagcactgctgggatacctttgtc |
| | | | gatcatcagggctgcccgttccagccgtgggatggtctggatgagcacag |
| | | | ccaggcgttaagcggtcgcctcagagcgattctgcaaaaccaaggtaatt |
| | | | ctggaagtgagacacccgggacttcagaatccgcgaccccggagtctgga |
| | | | atggataaaaagtattcaataggactagctattggcaccaacagcgttgg |
| | | | ttgggcagttatcaccgatgaatataaggtcccgagcaagaagtttaagg |
| | | | tcctgggcaacaccgatcgccacagcataaaaaagaaccttattggtgcg |
| | | | ctgctgttcgattctggtgagactgccgaagcgacccgtctgaagagaac |
| | | | cgcgcgtcgtagatacacccggcgcaagaaccgtatctgctatctgcaag |
| | | | aaattttcagcaacgagatggctaaggtcgacgactccttctttcatcgc |
| | | | ctggaggaaagctttctggttgaggaagacaaaaagcacgagcgtcatcc |
| | | | gattttcggtaacattgttgatgaagttgcctaccacgaaaaatacccca |
| | | | caatttatcatctgcgtaaaaaactggtagactccaccgataaggcggat |
| | | | ttacgccttatctatttggcgctggcgcatatgattaagttccgcggtca |
| | | | ttttttgatcgagggcgacctgaatccggataacagcgacgttgacaaac |
| | | | tgtttatccagctggtgcagacctataaccagttgttcgaagagaacccg |
| | | | attaatgcgtctggcgtggacgcgaaggcgatcctgagcgctcgtctgtc |
| | | | caagtcacgccgcctggagaacttgatcgcgcaactgccgggtgagaaga |
| | | | aaaacggcttgttcggcaatctgattgctctgtccctaggtttgaccccg |
| | | | aatttcaaatctaattttgatctggcggaagatgcgaaattacagttgag |
| | | | caaggacacttacgatgacgatctggacaacctgctcgctcagatcggcg |
| | | | atcagtatgccgatcttttcctggccgcgaagaacctgagcgatgccatc |
| | | | ttgctatcggacatcttacgtgttaacaccgaaatcaccaaagctccgct |
| | | | aagcgcgtccatgattaaacgttacgatgaacaccaccaggatctgacgc |
| | | | tgctgaaagcgttggtgcgccagcaactgccggaaaaatacaaggaaatt |
| | | | ttcttcgaccaaagcaagaacggttatgcaggctatatcgacggtggtgc |
| | | | gtcgcaagaagaattttataaattcatcaaaccgattttggagaagatgg |
| | | | atggtaccgaggagctgctggtaaaattgaatcgggaagacctcttgcga |
| | | | aagcagcgtactttcgataatggtagcatcccgcaccagattcatctggg |
| | | | cgaacttcacgcaatcctacgtcgtcaagaggatttctacccgtttctga |
| | | | aggataaccgtgaaaagatcgagaagattctgacctttcgtattccgtat |
| | | | tatgtgggtccgctggcgagaggcaacagccgttttgcgtggatgacccg |
| | | | caagtcggaagagactatcacgccgtggaatttcgaggaggtcgtggaca |
| | | | agggtgcatcagcgcaatcttttattgaacgtatgaccaattttgataaa |
| | | | aaccttccaaatgaaaaagtgttgccgaagcacagcttgctgtacgagta |
| | | | tttcaccgtttacaatgaactgacgaaagtcaagtacgttaccgaaggta |
| | | | tgcgtaagccagcctttctgtctggggagcaaaaaaaagccattgtggat |
| | | | ctgctgttcaagaccaatcgcaaggttaccgtcaagcaactgaaagagga |
| | | | ttacttcaaaaaaatcgaatgttttgactccgtggaaatcagcggcgttg |
| | | | aagaccgctttaatgcatctctgggcacctaccacgatctactcaagatc |
| | | | atcaaggataaggacttcctggacaacgaagaaaatgaggatatcttgga |
| | | | agatattgtgctgactttaacgttgttcgaggatcgtgagatgattgaag |
| | | | aacgcctcaaaacgtacgcacatctgtttgacgataaggtgatgaaacag |
| | | | ctaaagcgccgtcgttacaccggttggggtcgtctgtctagaaagctgat |
| | | | taacggcattcgtgacaaacaatctggcaagaccatcctggacttcctca |
| | | | aatccgacggcttcgcaaatcgtaatttcatgcaactgatccatgatgat |
| | | | agcctgacctttaaagaagatattcagaaggctcaagtgtccggccaggg |
| | | | tgactcgctgcatgagcatatcgcgaatctggctggctcccctgcgataa |
| | | | aaaaaggtattctccaaacggtgaaagttgtggatgaactggttaaagtc |
| | | | atgggtcgtcataaaccggaaaacatcgtgattgagatggcacgtgagaa |
| | | | ccagaccacccagaaaggtcaaaaaaacagtcgtgagcgtatgaaacgta |
| | | | tcgaagaggggattaaggagttaggcagccagattctgaaggagcacccg |
| | | | gttgagaacacccagctgcaaaacgagaagctctacctgtattacctgca |
| | | | aaacggccgtgacatgtatgttgaccaggagctggacatcaatcgtctca |
| | | | gcgactacgacgtggatgctatcgtgccgcagagctttctaaaagatgat |
| | | | tccatcgacaataaagtgctcacccgtagcgacaagaaccgtggtaaaag |
| | | | cgacaacgtgccgagcgaggaagtagttaagaaaatgaaaaactactggc |
| | | | gtcagctattgaacgccaagcttatcacccagcgtaagtttgacaacttg |
| | | | acgaaggcagagcgtggcggcctgagcgagctagacaaggcgggttttat |
| | | | taaacgccagctggtggaaactcgccagattaccaaacacgttgcgcaaa |
| | | | tcttggactcacgcatgaatacgaagtacgacgaaaacgataaattgatc |
| | | | cgcgaggtgaaggtcataaccctgaaatctaaactggttagcgatttccg |
| | | | gaaggactttcaattttataaagtccgggagattaacaactatcaccatg |
| | | | cacacgacgcttacttaaatgcggttgttggcacggccctgatcaagaag |
| | | | tacccgaaactggaaagcgagtttgtttatggtgactacaaggtgtatga |
| | | | cgttcgcaagatgattgcaaaatccgaacaagagattggtaaggcgacgg |
| | | | ctaagtattttttctacagcaatatcatgaacttcttcaagactgaaatc |
| | | | accctggcgaacggtgaaattcgtaaacgtccgttgatcgaaaccaacgg |
| | | | tgaaaccggtgaaatcgtttgggataaaggtagggattttgcgaccgttc |
| | | | gtaaggtcctgagcatgccgcaggttaacatcgtgaaaaagaccgaagtg |
| | | | cagaccggcggtttctccaaggagagtatcttaccgaagcgcaacagtga |
| | | | caagctgatcgcgcgtaaaaaagactgggatccgaaaaaatacggcggct |
| | | | ttgactccccgaccgtcgcgtacagcgtactggtcgtggctaaagtggag |
| | | | aaaggcaaatcgaaaaagctgaaatccgttaaagagttgctgggtattac |
| | | | gattatggaacgttcgagcttcgagaaaaatccgatcgacttcctggagg |
| | | | ccaaaggctacaaggaagttaaaaaagacctgatcattaaactgccgaag |
| | | | tacagcctgttcgagctcgagaacggtcgcaaacgcatgctggcgagcgc |
| | | | gggtgagctccaaaaaggcaacgaattggctctgccgagcaagtacgtga |
| | | | acttcctgtatctggcctcccattatgaaaaactgaagggtagcccggaa |
| | | | gataacgaacaaaagcagctgttcgtggaacaacataaacactatctgga |
| | | | cgagattattgagcaaatcagcgagttcagcaaacgcgtgattctggcgg |
| | | | atgctaatctggacaaggttctttcagcctataataaacaccgtgataag |
| | | | ccgatccgtgaacaggcagaaaatattattcacctgttcaccctgacgaa |
| | | | cctgggtgcgccagcagcgtttaaatacttcgacacaacgatcgaccgga |
| | | | agcgttataccagcaccaaagaggttcttgacgccaccttgattcaccaa |
| | | | agcatcacgggtctgtatgaaacccgtattgacttgagccagttgggcgg |
| | | | agattcagggggaagtggcggaagcggtgggtctacgaacctgagcgaca |
| | | | ttattgaaaaagaaactggtaaacagttggtgatccaagaatccattctg |
| | | | atgttaccggaggaagtggaagaggtgatcggcaacaagccggagtccga |
| | | | catcctggttcacaccgcgtatgatgagtcgaccgatgagaatgttatgc |
| | | | tgctgaccagcgacgccccagagtacaaaccgtgggcgctcgtcatccag |
| | | | gatagcaatggcgaaaacaagatcaagatgttgtctggcggtagcatggc |
| | | | tccgaaaaagaagcgtaaagtaggtattcatggtgttccggcatcaggtt |
| | | | aa |
| 2 | Base editor | A3A-dCasPhi-2 | atgggaagtcatcaccaccaccaccattcaggtgagaacctgtacttcca |
| | | | gagcatggctccgaaaaagaaacgtaaggtgggtatccacggcgttccgg |
| | | | cgatggaagcaagtccagcttcaggacccaggcacctgatggacccgcat |
| | | | attttcacctctaattttaacaacggtattggccgtcacaagacgtacct |
| | | | gtgttatgaagtggaacgtcttgacaacggcacctccgtgaaaatggacc |
| | | | agcatcgtggtttcttgcataatcaagctaaaaacctgctgtgcggtttc |
| | | | tacggccgtcacgcggaactgcgttttttggacctggttccgagcctgca |
| | | | actggacccggcacagatttatcgtgttacttggttcatcagctggtcgc |
| | | | cgtgttttagctggggttgcgccggcgaggtacgcgcgtttttgcaagag |
| | | | aacacccacgtgcgtttgcgcatcttcgccgctcgtatctacgattatga |
| | | | cccactgtataaagaagcgctccagatgctgcgcgatgcaggcgcgcagg |
| | | | tttccatcatgacctacgacgagttcaagcactgctgggatacctttgtc |
| | | | gatcatcagggctgcccgttccagccgtgggatggtctggatgagcacag |
| | | | ccaggcgttaagcggtcgcctcagagcgattctgcaaaaccaaggtaatt |
| | | | ctggaagtgagacacccgggacttcagaatccgcgaccccggagtctgga |
| | | | atgccaaaacccgctgtagagtcagaattttcaaaagttttgaaaaagca |
| | | | ctttccgggagaacgtttccgctcgagctatatgaaaagaggcggtaaga |
| | | | tcctggctgcccaaggcgaagaggcagttgttgcgtatctacagggcaag |
| | | | tccgaggaggagcctccgaacttccagccaccggcgaagtgccacgtggt |
| | | | gaccaaatcccgtgacttcgcggaatggccgatcatgaaggcgtcggaag |
| | | | cgattcaacgttatatctatgcactgtcgaccaccgaacgcgcggcctgt |
| | | | aaaccgggcaaaagtagtgagagccatgcagcgtggttcgccgctaccgg |
| | | | tgtcagcaaccacggctacagccacgttcagggtctgaacctgatttttg |
| | | | atcataccttgggccgttacgatggtgttctgaaaaaggtccaactgcgc |
| | | | aacgaaaaggcgagagcgcgtttagagagcattaacgcgtctagagcaga |
| | | | tgaaggtttaccggagattaaagccgaggaggaagaagtggctacgaacg |
| | | | aaaccggccacctgttgcaaccgccgggtatcaatccgagcttttacgtg |
| | | | tatcagaccattagcccgcaggcataccgtccgcgtgacgagatcgtgct |
| | | | gccgccggagtacgcaggttacgtacgtgacccgaatgctccgattccgc |
| | | | tgggtgtggtgcgtaaccgttgcgatatccaaaaagggtgcccgggctac |
| | | | atcccggaatggcagcgtgaagcaggtaccgcgatcagcccgaaaacagg |
| | | | caaggccgtaactgtgccgggcctgtctccgaagaaaaacaaacgtatgc |
| | | | gtaggtactggcgctctgagaaggagaaagctcaagatgcgctgttggtt |
| | | | accgtacgcattggtaccgattgggttgtcatcgacgtgcgcggtctgtt |
| | | | gcgcaacgcccgttggcgcaccatcgctcccaaggacatcagcctgaatg |
| | | | cgctcctcgatttgtttaccggtgatccggtgatcgatgtgcgtcgcaat |
| | | | attgttacctttacgtacaccctcgacgcctgtggtacctacgcccgtaa |
| | | | atggaccttgaaaggcaaacagacgaaagcgaccctggacaaactgacgg |
| | | | ccactcagacggttgcgttggttgcgattgcgcttggtcagaccaacccg |
| | | | atttccgctggcattagccgtgttacacaagaaaatggtgcactgcaatg |
| | | | cgaaccgctggaccggttcaccctgccggatgacctgttgaaggacatca |
| | | | gcgcatatcgcattgcttgggacagaaacgaagaggaactacgcgctcgc |
| | | | agcgtcgaggccctgccggaggcgcaacaagcggaggtccgtgcgctgga |
| | | | cggggtgtccaaagaaaccgcgcgtacccaactgtgcgcagatttcggcc |
| | | | tggacccgaaacgtctgccgtgggataaaatgagctccaatactaccttt |
| | | | atcagcgaagccttgctgtctaacagcgtgagccgtgatcaggttttttt |
| | | | cactccggctccgaagaagggtgctaagaaaaaagcgcctgttgaagtta |
| | | | tgcgtaaagaccgcacttgggcacgtgcgtacaagccgcgtctgagcgtc |
| | | | gaagctcagaaactgaagaacgaagccctgtgggcacttaagcgcacctc |
| | | | cccggagtatttgaagctgagccgtcgcaaagaggagctgtgtcgtcgta |
| | | | gcatcaactatgttatcgaaaagacccgtcgccgtacgcaatgtcagatt |
| | | | gtgatcccggtgatcgaggatctgaatgttcgcttctttcatggttctgg |
| | | | taagagactgcccggttgggataatttcttcaccgctaaaaaagagaacc |
| | | | gttggttcatccagggcctgcacaaagcgtttagcgatctgcgtacgcac |
| | | | cgttccttctatgtttttgaagttcgtccggagcgcacctctattacctg |
| | | | tccaaagtgcggtcattgcgaggtgggtaatcgtgacggtgaggcgtttc |
| | | | agtgcctgagctgcggcaagacgtgcaacgcagatcttgacgtcgcgacc |
| | | | cataatctgacccaagtggcgttgaccggtaagaccatgccgaagcgcga |
| | | | ggagccgcgtgacgctcagggtacagcgccagcgcgcaagactaagaagg |
| | | | cgagcaaatcaaaggcgccaccggcggagcgcgaagaccagaccccggcg |
| | | | caagagccaagccagaccagttctggaggtagcgggggtagcggtggtag |
| | | | cactaacttaagtgatattatcgagaaagaaaccgggaaacagctggtga |
| | | | tccaagaaagcattctgatgctgcccgaagaagttgaagaagtgattggt |
| | | | aacaagcctgagtcggacatcttagtgcacaccgcgtacgatgaaagcac |
| | | | ggatgagaatgtgatgctgctcacctctgatgcccccgaatacaagccgt |
| | | | gggctctggtgatccaggactctaacggagaaaacaaaattaagatgctc |
| | | | tctggcggctccatggcgcctaaaaaaaaacgcaaagtggggattcacgg |
| | | | ggttccggcgtaa |
| 3 | Base editor | A3A- | atgggaagtcatcaccaccaccaccattcaggtgagaacctgtacttcca |
| | | dLbCas12 | gagcatggctccgaaaaaagaaacgtaaggtgggtatccacggcgttccg |
| | | | gcgatggaagcaagtcca |
| | | | gcttcaggacccaggcacctgatggacccgcatattttcacctctaattt |
| | | | taacaacggtattggccgtcacaagacgtacctgtgttatgaagtggaac |
| | | | gtcttgacaacggcacctccgtgaaaatggaccagcatcgtggtttcttg |
| | | | cataatcaagctaaaaacctgctgtgcggtttctacggccgtcacgcgga |
| | | | actgcgttttttggacctggttccgagcctgcaactggacccggcacaga |
| | | | tttatcgtgttacttggttcatcagctggtcgccgtgttttagctggggt |
| | | | tgcgccggcgaggtacgcgcgtttttgcaagagaacacccacgtgcgttt |
| | | | gcgcatcttcgccgctcgtatctacgattatgacccactgtataaagaag |
| | | | cgctccagatgctgcgcgatgcaggcgcgcaggtttccatcatgacctac |
| | | | gacgagttcaagcactgctgggatacctttgtcgatcatcagggctgccc |
| | | | gttccagccgtgggatggtctggatgagcacagccaggcgttaagcggtc |
| | | | gcctcagagcgattctgcaaaaccaaggtaattctggaagtgagacaccc |
| | | | gggacttcagaatccgcgaccccggagtctggaatgtcaaaactagaaaa |
| | | | gtttacaaattgttactccctgtcgaaaaccctgagattcaaagcgatcc |
| | | | cggtgggtaaaactcaagagaacattgataacaaacgtctgctggttgag |
| | | | gacgaaaagcgcgcggaggattacaaaggcgttaaaaagctgttggaccg |
| | | | ttattacctgtcctttattaatgatgttctgcacagcattaaattaaaga |
| | | | acctaaacaattacatctccctcttccgtaaaaagacccgcaccgaaaag |
| | | | gaaaataaagaactggagaaccttgagattaatctgcgtaaagaaatcgc |
| | | | taaagcgtttaaaggcaacgagggttataaatccttatttaaaaaagaca |
| | | | tcatcgaaacgattctcccagaattcctggacgacaaagacgagatcgcg |
| | | | ctggttaattccttcaacggctttaccaccgcattcaccggcttttttga |
| | | | taaccgcgaaaacatgttctctgaagaggccaaaagcacctcaattgcgt |
| | | | ttcgctgcattaacgaaaatctgacgcgttatatctctaacatggatatc |
| | | | ttcgagaaggttgacgctatctttgataaacatgaagtccaggagatcaa |
| | | | ggagaagatcctgaacagcgactacgacgtggaggatttcttcgagggtg |
| | | | agttcttcaattttgttttgacccaagaaggcatcgatgtctacaatgcg |
| | | | ataattggtggttttgttaccgagagcggggagaaaattaagggtctgaa |
| | | | cgaatatatcaacttgtacaaccagaaaaccaaacaaaaactgccgaagt |
| | | | ttaagccgctgtacaagcaagtcttgtcggatcgtgaatcgctgagcttt |
| | | | tacggcgaaggctacaccagcgatgaggaggtgttggaggtgttccgcaa |
| | | | tacgttgaataagaactctgaaattttttccagcattaagaagctggaaa |
| | | | aactattcaagaacttcgacgagtatagcagcgcaggtatctttgtgaaa |
| | | | aacggtccggctattagcaccatctcgaaagacattttcggcgaatggaa |
| | | | tgttattcgtgataagtggaatgccgaatacgacgacattcatctgaaga |
| | | | aaaaggcggttgtaactgaaaagtacgaggacgatcgtcgtaagtcgttc |
| | | | aaaaagatcggctccttctctctggagcaactgcaagagtacgccgatgc |
| | | | cgatttgtcagtcgtggagaagctgaaagagatcatcattcaaaaagttg |
| | | | acgaaatctataaggtctatggtagcagtgaaaaactgtttgacgcggac |
| | | | ttcgtgctggagaaaagcctgaagaaaaatgacgccgtggtggcaatcat |
| | | | gaaagatcttctggatagcgtcaagtctttcgaaaactacatcaaagcgt |
| | | | ttttcggtgaaggcaaggaaacgaatcgtgacgagagcttctatggtgat |
| | | | ttcgtcctagcctatgatatcctgcttaaggtggatcacatctatgatgc |
| | | | catccgcaactatgttacccagaaaccgtactcaaaggacaagttcaagt |
| | | | tatacttccagaatccgcagtttatgggtggctgggataaagataaggaa |
| | | | accgactatcgcgccaccattttacgttacggtagtaagtactatctggc |
| | | | aatcatggataaaaaatatgcgaaatgcctgcaaaagatcgacaaggacg |
| | | | atgttaacggcaattatgagaagatcaactacaaactgctgccgggccca |
| | | | aataagatgctgccgaaggtgtttttcagcaaaaaatggatggcgtatta |
| | | | caatccgagcgaggacatccaaaagatctacaaaaacggtacgtttaaga |
| | | | agggtgatatgttcaacctcaacgattgtcataaacttattgacttcttc |
| | | | aaggatagcatatctcgttatccgaagtggagcaacgcgtatgactttaa |
| | | | ctttagcgaaaccgagaaatacaaagatatcgcgggtttctatcgtgaag |
| | | | ttgaagaacagggttacaaagtgtccttcgagtccgcgagcaagaaggag |
| | | | gtagacaagctggtcgaggagggtaagttgtacatgtttcagatttacaa |
| | | | taaggacttttccgacaagtcccatggtacaccgaatttgcacaccatgt |
| | | | atttcaagcttctgttcgacgagaacaaccacggccagatccgtctgagc |
| | | | ggtggtgcagagctgttcatgcgtagagctagcctgaagaaagaagagtt |
| | | | ggtcgttcatccggctaattcgccgatcgcgaacaaaaatccagataacc |
| | | | ctaaaaagactacgaccctgagctatgatgtgtataaagacaaacgcttt |
| | | | agcgaagaccagtatgagttacacattccgatcgctatcaacaagtgccc |
| | | | gaagaatattttcaagatcaacaccgaagtgcgtgttcttttgaagcacg |
| | | | atgacaacccgtatgtgattggtattgctcgtggtgaacgtaatctgctg |
| | | | tacattgtggttgtggacggcaaaggaaacatcgtggaacagtacagcct |
| | | | gaacgaaatcattaacaatttcaacggcattcgcattaagacggattacc |
| | | | attctttgctggacaaaaaggagaaagagcgtttcgaggcccgtcagaac |
| | | | tggacctccatcgagaacattaaggagctgaaggcgggttacatcagcca |
| | | | ggtggtgcacaaaatctgcgaactggtggagaagtacgacgcggtgattg |
| | | | ctttggcggatttgaactccggttttaagaacagccgcgttaaagtggaa |
| | | | aagcaggtttatcagaaattcgaaaaaatgctgattgataaattgaatta |
| | | | catggttgacaaaaagtctaacccgtgtgcaaccggcggtgcgctgaagg |
| | | | gctaccaaatcaccaataagtttgaaagctttaagagcatgagcacgcaa |
| | | | aatggcttcattttttacattccggcgtggctgaccagcaagatcgatcc |
| | | | gtcaaccggctttgttaatttactgaagacgaagtatacctctattgccg |
| | | | actctaaaaaatttattagttccttcgatagaatcatgtatgttccggaa |
| | | | gaggacctgttcgaatttgcactggactacaagaactttagccgtaccga |
| | | | tgctgattatataaagaaatggaaactctacagctatggcaaccgcatcc |
| | | | gtatctttcgcaacccgaaaaaaaacaacgtgttcgactgggaagaggta |
| | | | tgtctgacttcagcgtacaaggagcttttcaataaatatggtatcaacta |
| | | | ccagcagggtgatatccgtgcactgctttgcgagcaaagcgataaagcgt |
| | | | tctattcaagcttcatggcgctgatgagcctgatgctgcaaatgcgcaac |
| | | | agcatcaccggtcgtaccgatgttgcatttctgatttctccagtaaaaaa |
| | | | cagcgatggtatcttttacgacagccgtaattacgaggcccaagaaaatg |
| | | | cgattttgccgaagaacgctgacgcgaatggtgcatacaacattgcaagg |
| | | | aaagttctgtgggcaatcggccaatttaaaaaagcggaagatgagaagct |
| | | | ggacaaagtgaaaattgcgattagcaacaaggagtggctggagtatgcgc |
| | | | agacctccgttaaatcagggggaagtggcggaagcggtgggtctacgaac |
| | | | ctgagcgacattattgaaaaagaaactggtaaacagttggtgatccaaga |
| | | | atccattctgatgttaccggaggaagtggaagaggtgatcggcaacaagc |
| | | | cggagtccgacatcctggttcacaccgcgtatgatgagtcgaccgatgag |
| | | | aatgttatgctgctgaccagcgacgccccagagtacaaaccgtgggcgct |
| | | | cgtcatccaggatagcaatggcgaaaacaagatcaagatgttgtctggcg |
| | | | gtagcatggctccgaaaaagaagcgtaaagtaggtattcatggtgttccg |
| | | | gcatcaggttaa |
| 4 | Base editor | A3A-Y130F- | atgggaagtcatcaccaccaccaccattcaggtgagaacctgtacttcca |
| | | Cas9 | gagcatggctccgaaaaadgaaacgtaaggtgggtatccacggcgttccg |
| | | | gcgatggaagcatcaccag |
| | | | ctagtggacccaggcacctgatggatccgcacatcttcacctccaatttt |
| | | | aataacggcatcggccgtcataagacgtacctgtgctatgaggttgaacg |
| | | | tttggacaacggtactagcgtcaagatggaccagcatcgtggtttccttc |
| | | | acaaccaagcgaaaaatctgctgtgcggtttttatggtcgtcacgccgaa |
| | | | ctgagattcttggatctggttccgtctctgcaactggacccggcgcagat |
| | | | ttatcgtgtgacctggttcatcagctggtcgccgtgctttagctggggtt |
| | | | gcgccggtgaagtgcgcgcatttctccaggagaacacccatgttcgtttg |
| | | | cgcatcttcgcggcacgtatcttcgattacgacccgctgtacaaggaggc |
| | | | gctgcaaatgttgcgtgatgctggcgctcaagttagcattatgacctacg |
| | | | acgaatttaaacactgttgggataccttcgtggatcatcagggctgtccg |
| | | | tttcagccatgggacggcctggacgagcacagccaggcgttatccggtcg |
| | | | cctgcgcgcgattctgcaaaaccagggaaactctggaagtgagacacccg |
| | | | ggacttcagaatccgcgaccccggagtctggaatggataaaaagtattca |
| | | | ataggactagctattggcaccaacagcgttggttgggcagttatcaccga |
| | | | tgaatataaggtcccgagcaagaagtttaaggtcctgggcaacaccgatc |
| | | | gccacagcataaaaaagaaccttattggtgcgctgctgttcgattctggt |
| | | | gagactgccgaagcgacccgtctgaagagaaccgcgcgtcgtagatacac |
| | | | ccggcgcaagaaccgtatctgctatctgcaagaaattttcagcaacgaga |
| | | | tggctaaggtcgacgactccttctttcatcgcctggaggaaagctttctg |
| | | | gttgaggaagacaaaaagcacgagcgtcatccgattttcggtaacattgt |
| | | | tgatgaagttgcctaccacgaaaaataccccacaatttatcatctgcgta |
| | | | aaaaactggtagactccaccgataaggcggatttacgccttatctatttg |
| | | | gcgctggcgcatatgattaagttccgcggtcattttttgatcgagggcga |
| | | | cctgaatccggataacagcgacgttgacaaactgtttatccagctggtgc |
| | | | agacctataaccagttgttcgaagagaacccgattaatgcgtctggcgtg |
| | | | gacgcgaaggcgatcctgagcgctcgtctgtccaagtcacgccgcctgga |
| | | | gaacttgatcgcgcaactgccgggtgagaagaaaaacggcttgttcggca |
| | | | atctgattgctctgtccctaggtttgaccccgaatttcaaatctaatttt |
| | | | gatctggcggaagatgcgaaattacagttgagcaaggacacttacgatga |
| | | | cgatctggacaacctgctcgctcagatcggcgatcagtatgccgatcttt |
| | | | tcctggccgcgaagaacctgagcgatgccatcttgctatcggacatctta |
| | | | cgtgttaacaccgaaatcaccaaagctccgctaagcgcgtccatgattaa |
| | | | acgttacgatgaacaccaccaggatctgacgctgctgaaagcgttggtgc |
| | | | gccagcaactgccggaaaaatacaaggaaattttcttcgaccaaagcaag |
| | | | aacggttatgcaggctatatcgacggtggtgcgtcgcaagaagaatttta |
| | | | taaattcatcaaaccgattttggagaagatggatggtaccgaggagctgc |
| | | | tggtaaaattgaatcgggaagacctcttgcgaaagcagcgtactttcgat |
| | | | aatggtagcatcccgcaccagattcatctgggcgaacttcacgcaatcct |
| | | | acgtcgtcaagaggatttctacccgtttctgaaggataaccgtgaaaaga |
| | | | tcgagaagattctgacctttcgtattccgtattatgtgggtccgctggcg |
| | | | agaggcaacagccgttttgcgtggatgacccgcaagtcggaagagactat |
| | | | cacgccgtggaatttcgaggaggtcgtggacaagggtgcatcagcgcaat |
| | | | cttttattgaacgtatgaccaattttgataaaaaccttccaaatgaaaaa |
| | | | gtgttgccgaagcacagcttgctgtacgagtatttcaccgtttacaatga |
| | | | actgacgaaagtcaagtacgttaccgaaggtatgcgtaagccagcctttc |
| | | | tgtctggggagcaaaaaaaagccattgtggatctgctgttcaagaccaat |
| | | | cgcaaggttaccgtcaagcaactgaaagaggattacttcaaaaaaatcga |
| | | | atgttttgactccgtggaaatcagcggcgttgaagaccgctttaatgcat |
| | | | ctctgggcacctaccacgatctactcaagatcatcaaggataaggacttc |
| | | | ctggacaacgaagaaaatgaggatatcttggaagatattgtgctgacttt |
| | | | aacgttgttcgaggatcgtgagatgattgaagaacgcctcaaaacgtacg |
| | | | cacatctgtttgacgataaggtgatgaaacagctaaagcgccgtcgttac |
| | | | accggttggggtcgtctgtctagaaagctgattaacggcattcgtgacaa |
| | | | acaatctggcaagaccatcctggacttcctcaaatccgacggcttcgcaa |
| | | | atcgtaatttcatgcaactgatccatgatgatagcctgacctttaaagaa |
| | | | gatattcagaaggctcaagtgtccggccagggtgactcgctgcatgagca |
| | | | tatcgcgaatctggctggctcccctgcgataaaaaaaggtattctccaaa |
| | | | cggtgaaagttgtggatgaactggttaaagtcatgggtcgtcataaaccg |
| | | | gaaaacatcgtgattgagatggcacgtgagaaccagaccacccagaaagg |
| | | | tcaaaaaaacagtcgtgagcgtatgaaacgtatcgaagaggggattaagg |
| | | | agttaggcagccagattctgaaggagcacccggttgagaacacccagctg |
| | | | caaaacgagaagctctacctgtattacctgcaaaacggccgtgacatgta |
| | | | tgttgaccaggagctggacatcaatcgtctcagcgactacgacgtggatg |
| | | | ctatcgtgccgcagagctttctaaaagatgattccatcgacaataaagtg |
| | | | ctcacccgtagcgacaagaaccgtggtaaaagcgacaacgtgccgagcga |
| | | | ggaagtagttaagaaaatgaaaaactactggcgtcagctattgaacgcca |
| | | | agcttatcacccagcgtaagtttgacaacttgacgaaggcagagcgtggc |
| | | | ggcctgagcgagctagacaaggcgggttttattaaacgccagctggtgga |
| | | | aactcgccagattaccaaacacgttgcgcaaatcttggactcacgcatga |
| | | | atacgaagtacgacgaaaacgataaattgatccgcgaggtgaaggtcata |
| | | | accctgaaatctaaactggttagcgatttccggaaggactttcaatttta |
| | | | taaagtccgggagattaacaactatcaccatgcacacgacgcttacttaa |
| | | | atgcggttgttggcacggccctgatcaagaagtacccgaaactggaaagc |
| | | | gagtttgtttatggtgactacaaggtgtatgacgttcgcaagatgattgc |
| | | | aaaatccgaacaagagattggtaaggcgacggctaagtattttttctaca |
| | | | gcaatatcatgaacttcttcaagactgaaatcaccctggcgaacggtgaa |
| | | | attcgtaaacgtccgttgatcgaaaccaacggtgaaaccggtgaaatcgt |
| | | | ttgggataaaggtagggattttgcgaccgttcgtaaggtcctgagcatgc |
| | | | cgcaggttaacatcgtgaaaaagaccgaagtgcagaccggcggtttctcc |
| | | | aaggagagtatcttaccgaagcgcaacagtgacaagctgatcgcgcgtaa |
| | | | aaaagactgggatccgaaaaaatacggcggctttgactccccgaccgtcg |
| | | | cgtacagcgtactggtcgtggctaaagtggagaaaggcaaatcgaaaaag |
| | | | ctgaaatccgttaaagagttgctgggtattacgattatggaacgttcgag |
| | | | cttcgagaaaaatccgatcgacttcctggaggccaaaggctacaaggaag |
| | | | ttaaaaaagacctgatcattaaactgccgaagtacagcctgttcgagctc |
| | | | gagaacggtcgcaaacgcatgctggcgagcgcgggtgagctccaaaaagg |
| | | | caacgaattggctctgccgagcaagtacgtgaacttcctgtatctggcct |
| | | | cccattatgaaaaactgaagggtagcccggaagataacgaacaaaagcag |
| | | | ctgttcgtggaacaacataaacactatctggacgagattattgagcaaat |
| | | | cagcgagttcagcaaacgcgtgattctggcggatgctaatctggacaagg |
| | | | ttctttcagcctataataaacaccgtgataagccgatccgtgaacaggca |
| | | | gaaaatattattcacctgttcaccctgacgaacctgggtgcgccagcagc |
| | | | gtttaaatacttcgacacaacgatcgaccggaagcgttataccagcacca |
| | | | aagaggttcttgacgccaccttgattcaccaaagcatcacgggtctgtat |
| | | | gaaacccgtattgacttgagccagttgggcggagattcagggggaagtgg |
| | | | cggaagcggtgggtctacgaacctgagcgacattattgaaaaagaaactg |
| | | | gtaaacagttggtgatccaagaatccattctgatgttaccggaggaagtg |
| | | | gaagaggtgatcggcaacaagccggagtccgacatcctggttcacaccgc |
| | | | gtatgatgagtcgaccgatgagaatgttatgctgctgaccagcgacgccc |
| | | | cagagtacaaaccgtgggcgctcgtcatccaggatagcaatggcgaaaac |
| | | | aagatcaagatgttgtctggcggtagcatggctccgaaaaagaagcgtaa |
| | | | agtaggtattcatggtgttccggcatcaggttaa |
| 5 | Base editor | A3A-Y130F- | atgggaagtcatcaccaccaccaccattcaggtgagaacctgtacttcca |
| | | dCasPhi-2 | gagcatggctccgaaaaagaaacgtaaggtgggtatccacggcgttccgg |
| | | | cgatggaagcatcaccagctagtggacccaggcacctgatggatccgcac |
| | | | atcttcacctccaattttaataacggcatcggccgtcataagacgtacct |
| | | | gtgctatgaggttgaacgtttggacaacggtactagcgtcaagatggacc |
| | | | agcatcgtggtttccttcacaaccaagcgaaaaatctgctgtgcggtttt |
| | | | tatggtcgtcacgccgaactgagattcttggatctggttccgtctctgca |
| | | | actggacccggcgcagatttatcgtgtgacctggttcatcagctggtcgc |
| | | | cgtgctttagctggggttgcgccggtgaagtgcgcgcatttctccaggag |
| | | | aacacccatgttcgtttgcgcatcttcgcggcacgtatcttcgattacga |
| | | | cccgctgtacaaggaggcgctgcaaatgttgcgtgatgctggcgctcaag |
| | | | ttagcattatgacctacgacgaatttaaacactgttgggataccttcgtg |
| | | | gatcatcagggctgtccgtttcagccatgggacggcctggacgagcacag |
| | | | ccaggcgttatccggtcgcctgcgcgcgattctgcaaaaccagggaaact |
| | | | ctggaagtgagacacccgggacttcagaatccgcgaccccggagtctgga |
| | | | atgccaaaacccgctgtagagtcagaattttcaaaagttttgaaaaagca |
| | | | ctttccgggagaacgtttccgctcgagctatatgaaaagaggcggtaaga |
| | | | tcctggctgcccaaggcgaagaggcagttgttgcgtatctacagggcaag |
| | | | tccgaggaggagcctccgaacttccagccaccggcgaagtgccacgtggt |
| | | | gaccaaatcccgtgacttcgcggaatggccgatcatgaaggcgtcggaag |
| | | | cgattcaacgttatatctatgcactgtcgaccaccgaacgcgcggcctgt |
| | | | aaaccgggcaaaagtagtgagagccatgcagcgtggttcgccgctaccgg |
| | | | tgtcagcaaccacggctacagccacgttcagggtctgaacctgatttttg |
| | | | atcataccttgggccgttacgatggtgttctgaaaaaggtccaactgcgc |
| | | | aacgaaaaggcgagagcgcgtttagagagcattaacgcgtctagagcaga |
| | | | tgaaggtttaccggagattaaagccgaggaggaagaagtggctacgaacg |
| | | | aaaccggccacctgttgcaaccgccgggtatcaatccgagcttttacgtg |
| | | | tatcagaccattagcccgcaggcataccgtccgcgtgacgagatcgtgct |
| | | | gccgccggagtacgcaggttacgtacgtgacccgaatgctccgattccgc |
| | | | tgggtgtggtgcgtaaccgttgcgatatccaaaaagggtgcccgggctac |
| | | | atcccggaatggcagcgtgaagcaggtaccgcgatcagcccgaaaacagg |
| | | | caaggccgtaactgtgccgggcctgtctccgaagaaaaacaaacgtatgc |
| | | | gtaggtactggcgctctgagaaggagaaagctcaagatgcgctgttggtt |
| | | | accgtacgcattggtaccgattgggttgtcatcgacgtgcgcggtctgtt |
| | | | gcgcaacgcccgttggcgcaccatcgctcccaaggacatcagcctgaatg |
| | | | cgctcctcgatttgtttaccggtgatccggtgatcgatgtgcgtcgcaat |
| | | | attgttacctttacgtacaccctcgacgcctgtggtacctacgcccgtaa |
| | | | atggaccttgaaaggcaaacagacgaaagcgaccctggacaaactgacgg |
| | | | ccactcagacggttgcgttggttgcgattgcgcttggtcagaccaacccg |
| | | | atttccgctggcattagccgtgttacacaagaaaatggtgcactgcaatg |
| | | | cgaaccgctggaccggttcaccctgccggatgacctgttgaaggacatca |
| | | | gcgcatatcgcattgcttgggacagaaacgaagaggaactacgcgctcgc |
| | | | agcgtcgaggccctgccggaggcgcaacaagcggaggtccgtgcgctgga |
| | | | cggggtgtccaaagaaaccgcgcgtacccaactgtgcgcagatttcggcc |
| | | | tggacccgaaacgtctgccgtgggataaaatgagctccaatactaccttt |
| | | | atcagcgaagccttgctgtctaacagcgtgagccgtgatcaggttttttt |
| | | | cactccggctccgaagaagggtgctaagaaaaaagcgcctgttgaagtta |
| | | | tgcgtaaagaccgcacttgggcacgtgcgtacaagccgcgtctgagcgtc |
| | | | gaagctcagaaactgaagaacgaagccctgtgggcacttaagcgcacctc |
| | | | cccggagtatttgaagctgagccgtcgcaaagaggagctgtgtcgtcgta |
| | | | gcatcaactatgttatcgaaaagacccgtcgccgtacgcaatgtcagatt |
| | | | gtgatcccggtgatcgaggatctgaatgttcgcttctttcatggttctgg |
| | | | taagagactgcccggttgggataatttcttcaccgctaaaaaagagaacc |
| | | | gttggttcatccagggcctgcacaaagcgtttagcgatctgcgtacgcac |
| | | | cgttccttctatgtttttgaagttcgtccggagcgcacctctattacctg |
| | | | tccaaagtgcggtcattgcgaggtgggtaatcgtgacggtgaggcgtttc |
| | | | agtgcctgagctgcggcaagacgtgcaacgcagatcttgacgtcgcgacc |
| | | | cataatctgacccaagtggcgttgaccggtaagaccatgccgaagcgcga |
| | | | ggagccgcgtgacgctcagggtacagcgccagcgcgcaagactaagaagg |
| | | | cgagcaaatcaaaggcgccaccggcggagcgcgaagaccagaccccggcg |
| | | | caagagccaagccagaccagttctggaggtagcgggggtagcggtggtag |
| | | | cactaacttaagtgatattatcgagaaagaaaccgggaaacagctggtga |
| | | | tccaagaaagcattctgatgctgcccgaagaagttgaagaagtgattggt |
| | | | aacaagcctgagtcggacatcttagtgcacaccgcgtacgatgaaagcac |
| | | | ggatgagaatgtgatgctgctcacctctgatgcccccgaatacaagccgt |
| | | | gggctctggtgatccaggactctaacggagaaaacaaaattaagatgctc |
| | | | tctggcggctccatggcgcctaaaaaaaaacgcaaagtggggattcacgg |
| | | | ggttccggcgtaa |
| 6 | Base editor | A3A-Y130F- | atgggaagtcatcaccaccaccaccattcaggtgagaacctgtacttcca |
| | | dLbCas12a | gagcatggctccgaaaaagaaacgtaaggtgggtatccacggcgttccgg |
| | | | cgatggaagcatcaccagctagtggacccaggcacctgatggatccgcac |
| | | | atcttcacctccaattttaataacggcatcggccgtcataagacgtacct |
| | | | gtgctatgaggttgaacgtttggacaacggtactagcgtcaagatggacc |
| | | | agcatcgtggtttccttcacaaccaagcgaaaaatctgctgtgcggtttt |
| | | | tatggtcgtcacgccgaactgagattcttggatctggttccgtctctgca |
| | | | actggacccggcgcagatttatcgtgtgacctggttcatcagctggtcgc |
| | | | cgtgctttagctggggttgcgccggtgaagtgcgcgcatttctccaggag |
| | | | aacacccatgttcgtttgcgcatcttcgcggcacgtatcttcgattacga |
| | | | cccgctgtacaaggaggcgctgcaaatgttgcgtgatgctggcgctcaag |
| | | | ttagcattatgacctacgacgaatttaaacactgttgggataccttcgtg |
| | | | gatcatcagggctgtccgtttcagccatgggacggcctggacgagcacag |
| | | | ccaggcgttatccggtcgcctgcgcgcgattctgcaaaaccagggaaact |
| | | | ctggaagtgagacacccgggacttcagaatccgcgaccccggagtctgga |
| | | | atgtcaaaactagaaaagtttacaaattgttactccctgtcgaaaaccct |
| | | | gagattcaaagcgatcccggtgggtaaaactcaagagaacattgataaca |
| | | | aacgtctgctggttgaggacgaaaagcgcgcggaggattacaaaggcgtt |
| | | | aaaaagctgttggaccgttattacctgtcctttattaatgatgttctgca |
| | | | cagcattaaattaaagaacctaaacaattacatctccctcttccgtaaaa |
| | | | agacccgcaccgaaaaggaaaataaagaactggagaaccttgagattaat |
| | | | ctgcgtaaagaaatcgctaaagcgtttaaaggcaacgagggttataaatc |
| | | | cttatttaaaaaagacatcatcgaaacgattctcccagaattcctggacg |
| | | | acaaagacgagatcgcgctggttaattccttcaacggctttaccaccgca |
| | | | ttcaccggcttttttgataaccgcgaaaacatgttctctgaagaggccaa |
| | | | aagcacctcaattgcgtttcgctgcattaacgaaaatctgacgcgttata |
| | | | tctctaacatggatatcttcgagaaggttgacgctatctttgataaacat |
| | | | gaagtccaggagatcaaggagaagatcctgaacagcgactacgacgtgga |
| | | | ggatttcttcgagggtgagttcttcaattttgttttgacccaagaaggca |
| | | | tcgatgtctacaatgcgataattggtggttttgttaccgagagcggggag |
| | | | aaaattaagggtctgaacgaatatatcaacttgtacaaccagaaaaccaa |
| | | | acaaaaactgccgaagtttaagccgctgtacaagcaagtcttgtcggatc |
| | | | gtgaatcgctgagcttttacggcgaaggctacaccagcgatgaggaggtg |
| | | | ttggaggtgttccgcaatacgttgaataagaactctgaaattttttccag |
| | | | cattaagaagctggaaaaactattcaagaacttcgacgagtatagcagcg |
| | | | caggtatctttgtgaaaaacggtccggctattagcaccatctcgaaagac |
| | | | attttcggcgaatggaatgttattcgtgataagtggaatgccgaatacga |
| | | | cgacattcatctgaagaaaaaggcggttgtaactgaaaagtacgaggacg |
| | | | atcgtcgtaagtcgttcaaaaagatcggctccttctctctggagcaactg |
| | | | caagagtacgccgatgccgatttgtcagtcgtggagaagctgaaagagat |
| | | | catcattcaaaaagttgacgaaatctataaggtctatggtagcagtgaaa |
| | | | aactgtttgacgcggacttcgtgctggagaaaagcctgaagaaaaatgac |
| | | | gccgtggtggcaatcatgaaagatcttctggatagcgtcaagtctttcga |
| | | | aaactacatcaaagcgtttttcggtgaaggcaaggaaacgaatcgtgacg |
| | | | agagcttctatggtgatttcgtcctagcctatgatatcctgcttaaggtg |
| | | | gatcacatctatgatgccatccgcaactatgttacccagaaaccgtactc |
| | | | aaaggacaagttcaagttatacttccagaatccgcagtttatgggtggct |
| | | | gggataaagataaggaaaccgactatcgcgccaccattttacgttacggt |
| | | | agtaagtactatctggcaatcatggataaaaaatatgcgaaatgcctgca |
| | | | aaagatcgacaaggacgatgttaacggcaattatgagaagatcaactaca |
| | | | aactgctgccgggcccaaataagatgctgccgaaggtgtttttcagcaaa |
| | | | aaatggatggcgtattacaatccgagcgaggacatccaaaagatctacaa |
| | | | aaacggtacgtttaagaagggtgatatgttcaacctcaacgattgtcata |
| | | | aacttattgacttcttcaaggatagcatatctcgttatccgaagtggagc |
| | | | aacgcgtatgactttaactttagcgaaaccgagaaatacaaagatatcgc |
| | | | gggtttctatcgtgaagttgaagaacagggttacaaagtgtccttcgagt |
| | | | ccgcgagcaagaaggaggtagacaagctggtcgaggagggtaagttgtac |
| | | | atgtttcagatttacaataaggacttttccgacaagtcccatggtacacc |
| | | | gaatttgcacaccatgtatttcaagcttctgttcgacgagaacaaccacg |
| | | | gccagatccgtctgagcggtggtgcagagctgttcatgcgtagagctagc |
| | | | ctgaagaaagaagagttggtcgttcatccggctaattcgccgatcgcgaa |
| | | | caaaaatccagataaccctaaaaagactacgaccctgagctatgatgtgt |
| | | | ataaagacaaacgctttagcgaagaccagtatgagttacacattccgatc |
| | | | gctatcaacaagtgcccgaagaatattttcaagatcaacaccgaagtgcg |
| | | | tgttcttttgaagcacgatgacaacccgtatgtgattggtattgctcgtg |
| | | | gtgaacgtaatctgctgtacattgtggttgtggacggcaaaggaaacatc |
| | | | gtggaacagtacagcctgaacgaaatcattaacaatttcaacggcattcg |
| | | | cattaagacggattaccattctttgctggacaaaaaggagaaagagcgtt |
| | | | tcgaggcccgtcagaactggacctccatcgagaacattaaggagctgaag |
| | | | gcgggttacatcagccaggtggtgcacaaaatctgcgaactggtggagaa |
| | | | gtacgacgcggtgattgctttggcggatttgaactccggttttaagaaca |
| | | | gccgcgttaaagtggaaaagcaggtttatcagaaattcgaaaaaatgctg |
| | | | attgataaattgaattacatggttgacaaaaagtctaacccgtgtgcaac |
| | | | cggcggtgcgctgaagggctaccaaatcaccaataagtttgaaagcttta |
| | | | agagcatgagcacgcaaaatggcttcattttttacattccggcgtggctg |
| | | | accagcaagatcgatccgtcaaccggctttgttaatttactgaagacgaa |
| | | | gtatacctctattgccgactctaaaaaatttattagttccttcgatagaa |
| | | | tcatgtatgttccggaagaggacctgttcgaatttgcactggactacaag |
| | | | aactttagccgtaccgatgctgattatataaagaaatggaaactctacag |
| | | | ctatggcaaccgcatccgtatctttcgcaacccgaaaaaaaacaacgtgt |
| | | | tcgactgggaagaggtatgtctgacttcagcgtacaaggagcttttcaat |
| | | | aaatatggtatcaactaccagcagggtgatatccgtgcactgctttgcga |
| | | | gcaaagcgataaagcgttctattcaagcttcatggcgctgatgagcctga |
| | | | tgctgcaaatgcgcaacagcatcaccggtcgtaccgatgttgcatttctg |
| | | | atttctccagtaaaaaacagcgatggtatcttttacgacagccgtaatta |
| | | | cgaggcccaagaaaatgcgattttgccgaagaacgctgacgcgaatggtg |
| | | | catacaacattgcaaggaaagttctgtgggcaatcggccaatttaaaaaa |
| | | | gcggaagatgagaagctggacaaagtgaaaattgcgattagcaacaagga |
| | | | gtggctggagtatgcgcagacctccgttaaatcagggggaagtggcggaa |
| | | | gcggtgggtctacgaacctgagcgacattattgaaaaagaaactggtaaa |
| | | | cagttggtgatccaagaatccattctgatgttaccggaggaagtggaaga |
| | | | ggtgatcggcaacaagccggagtccgacatcctggttcacaccgcgtatg |
| | | | atgagtcgaccgatgagaatgttatgctgctgaccagcgacgccccagag |
| | | | tacaaaccgtgggcgctcgtcatccaggatagcaatggcgaaagttccgg |
| | | | catcaggttaa |
| 7 | Base editor | A3B-dCas9 | atgggaagtcatcaccaccaccaccattcaggtgagaacctgtacttcca |
| | | | gagcatggctccgaaaaagaaacgtaaggtgggtatccacggcgttccgg |
| | | | cgatggaaatactaaggtatttaatggatcccgacactttcaccttcaat |
| | | | ttcaacaacgatccattggtcttgcgccgtcgtcagacctacttgtgcta |
| | | | tgaggttgaacgtctggataacggtacgtgggttttaatggatcagcata |
| | | | tgggtttcctgtgtaatgaagcgaagaacctgctgtgcggtttttacggc |
| | | | cgtcatgcggaactgcgttttcttgacctggttccgagcctgcaactgga |
| | | | cccggctcagatttaccgtgttacctggtttatctcgtggagcccgtgtt |
| | | | ttagctggggctgcgcgggtgaagtgcgcgcgtttttgcaagagaacacc |
| | | | cacgtgcgtctccgcatcttcgccgcacgtatttacgattatgacccgct |
| | | | gtataaagaggccctgcaaatgctgagagatgctggcgcacaagtgagca |
| | | | ttatgacctacgacgagttcgagtactgctgggatacgttcgtgtatcgc |
| | | | caaggttgcccgtttcagccgtgggacggcctggaggagcacagccaggc |
| | | | gctctccggtcgtttgcgcgcgatcctgcaatctggaagtgagacacccg |
| | | | ggacttcagaatccgcgaccccggagtctggaatggataaaaagtattca |
| | | | ataggactagctattggcaccaacagcgttggttgggcagttatcaccga |
| | | | tgaatataaggtcccgagcaagaagtttaaggtcctgggcaacaccgatc |
| | | | gccacagcataaaaaagaaccttattggtgcgctgctgttcgattctggt |
| | | | gagactgccgaagcgacccgtctgaagagaaccgcgcgtcgtagatacac |
| | | | ccggcgcaagaaccgtatctgctatctgcaagaaattttcagcaacgaga |
| | | | tggctaaggtcgacgactccttctttcatcgcctggaggaaagctttctg |
| | | | gttgaggaagacaaaaagcacgagcgtcatccgattttcggtaacattgt |
| | | | tgatgaagttgcctaccacgaaaaataccccacaatttatcatctgcgta |
| | | | aaaaactggtagactccaccgataaggcggatttacgccttatctatttg |
| | | | gcgctggcgcatatgattaagttccgcggtcattttttgatcgagggcga |
| | | | cctgaatccggataacagcgacgttgacaaactgtttatccagctggtgc |
| | | | agacctataaccagttgttcgaagagaacccgattaatgcgtctggcgtg |
| | | | gacgcgaaggcgatcctgagcgctcgtctgtccaagtcacgccgcctgga |
| | | | gaacttgatcgcgcaactgccgggtgagaagaaaaacggcttgttcggca |
| | | | atctgattgctctgtccctaggtttgaccccgaatttcaaatctaatttt |
| | | | gatctggcggaagatgcgaaattacagttgagcaaggacacttacgatga |
| | | | cgatctggacaacctgctcgctcagatcggcgatcagtatgccgatcttt |
| | | | tcctggccgcgaagaacctgagcgatgccatcttgctatcggacatctta |
| | | | cgtgttaacaccgaaatcaccaaagctccgctaagcgcgtccatgattaa |
| | | | acgttacgatgaacaccaccaggatctgacgctgctgaaagcgttggtgc |
| | | | gccagcaactgccggaaaaatacaaggaaattttcttcgaccaaagcaag |
| | | | aacggttatgcaggctatatcgacggtggtgcgtcgcaagaagaatttta |
| | | | taaattcatcaaaccgattttggagaagatggatggtaccgaggagctgc |
| | | | tggtaaaattgaatcgggaagacctcttgcgaaagcagcgtactttcgat |
| | | | aatggtagcatcccgcaccagattcatctgggcgaacttcacgcaatcct |
| | | | acgtcgtcaagaggatttctacccgtttctgaaggataaccgtgaaaaga |
| | | | tcgagaagattctgacctttcgtattccgtattatgtgggtccgctggcg |
| | | | agaggcaacagccgttttgcgtggatgacccgcaagtcggaagagactat |
| | | | cacgccgtggaatttcgaggaggtcgtggacaagggtgcatcagcgcaat |
| | | | cttttattgaacgtatgaccaattttgataaaaaccttccaaatgaaaaa |
| | | | gtgttgccgaagcacagcttgctgtacgagtatttcaccgtttacaatga |
| | | | actgacgaaagtcaagtacgttaccgaaggtatgcgtaagccagcctttc |
| | | | tgtctggggagcaaaaaaaagccattgtggatctgctgttcaagaccaat |
| | | | cgcaaggttaccgtcaagcaactgaaagaggattacttcaaaaaaatcga |
| | | | atgttttgactccgtggaaatcagcggcgttgaagaccgctttaatgcat |
| | | | ctctgggcacctaccacgatctactcaagatcatcaaggataaggacttc |
| | | | ctggacaacgaagaaaatgaggatatcttggaagatattgtgctgacttt |
| | | | aacgttgttcgaggatcgtgagatgattgaagaacgcctcaaaacgtacg |
| | | | cacatctgtttgacgataaggtgatgaaacagctaaagcgccgtcgttac |
| | | | accggttggggtcgtctgtctagaaagctgattaacggcattcgtgacaa |
| | | | acaatctggcaagaccatcctggacttcctcaaatccgacggcttcgcaa |
| | | | atcgtaatttcatgcaactgatccatgatgatagcctgacctttaaagaa |
| | | | gatattcagaaggctcaagtgtccggccagggtgactcgctgcatgagca |
| | | | tatcgcgaatctggctggctcccctgcgataaaaaaaggtattctccaaa |
| | | | cggtgaaagttgtggatgaactggttaaagtcatgggtcgtcataaaccg |
| | | | gaaaacatcgtgattgagatggcacgtgagaaccagaccacccagaaagg |
| | | | tcaaaaaaacagtcgtgagcgtatgaaacgtatcgaagaggggattaagg |
| | | | agttaggcagccagattctgaaggagcacccggttgagaacacccagctg |
| | | | caaaacgagaagctctacctgtattacctgcaaaacggccgtgacatgta |
| | | | tgttgaccaggagctggacatcaatcgtctcagcgactacgacgtggatg |
| | | | ctatcgtgccgcagagctttctaaaagatgattccatcgacaataaagtg |
| | | | ctcacccgtagcgacaagaaccgtggtaaaagcgacaacgtgccgagcga |
| | | | ggaagtagttaagaaaatgaaaaactactggcgtcagctattgaacgcca |
| | | | agcttatcacccagcgtaagtttgacaacttgacgaaggcagagcgtggc |
| | | | ggcctgagcgagctagacaaggcgggttttattaaacgccagctggtgga |
| | | | aactcgccagattaccaaacacgttgcgcaaatcttggactcacgcatga |
| | | | atacgaagtacgacgaaaacgataaattgatccgcgaggtgaaggtcata |
| | | | accctgaaatctaaactggttagcgatttccggaaggactttcaatttta |
| | | | taaagtccgggagattaacaactatcaccatgcacacgacgcttacttaa |
| | | | atgcggttgttggcacggccctgatcaagaagtacccgaaactggaaagc |
| | | | gagtttgtttatggtgactacaaggtgtatgacgttcgcaagatgattgc |
| | | | aaaatccgaacaagagattggtaaggcgacggctaagtattttttctaca |
| | | | gcaatatcatgaacttcttcaagactgaaatcaccctggcgaacggtgaa |
| | | | attcgtaaacgtccgttgatcgaaaccaacggtgaaaccggtgaaatcgt |
| | | | ttgggataaaggtagggattttgcgaccgttcgtaaggtcctgagcatgc |
| | | | cgcaggttaacatcgtgaaaaagaccgaagtgcagaccggcggtttctcc |
| | | | aaggagagtatcttaccgaagcgcaacagtgacaagctgatcgcgcgtaa |
| | | | aaaagactgggatccgaaaaaatacggcggctttgactccccgaccgtcg |
| | | | cgtacagcgtactggtcgtggctaaagtggagaaaggcaaatcgaaaaag |
| | | | ctgaaatccgttaaagagttgctgggtattacgattatggaacgttcgag |
| | | | cttcgagaaaaatccgatcgacttcctggaggccaaaggctacaaggaag |
| | | | ttaaaaaagacctgatcattaaactgccgaagtacagcctgttcgagctc |
| | | | gagaacggtcgcaaacgcatgctggcgagcgcgggtgagctccaaaaagg |
| | | | caacgaattggctctgccgagcaagtacgtgaacttcctgtatctggcct |
| | | | cccattatgaaaaactgaagggtagcccggaagataacgaacaaaagcag |
| | | | ctgttcgtggaacaacataaacactatctggacgagattattgagcaaat |
| | | | cagcgagttcagcaaacgcgtgattctggcggatgctaatctggacaagg |
| | | | ttctttcagcctataataaacaccgtgataagccgatccgtgaacaggca |
| | | | gaaaatattattcacctgttcaccctgacgaacctgggtgcgccagcagc |
| | | | gtttaaatacttcgacacaacgatcgaccggaagcgttataccagcacca |
| | | | aagaggttcttgacgccaccttgattcaccaaagcatcacgggtctgtat |
| | | | gaaacccgtattgacttgagccagttgggcggagattcagggggaagtgg |
| | | | cggaagcggtgggtctacgaacctgagcgacattattgaaaaagaaactg |
| | | | gtaaacagttggtgatccaagaatccattctgatgttaccggaggaagtg |
| | | | gaagaggtgatcggcaacaagccggagtccgacatcctggttcacaccgc |
| | | | gtatgatgagtcgaccgatgagaatgttatgctgctgaccagcgacgccc |
| | | | cagagtacaaaccgtgggcgctcgtcatccaggatagcaatggcgaaaac |
| | | | aagatcaagatgttgtctggcggtagcatggctccgaaaaagaagcgtaa |
| | | | agtaggtattcatggtgttccggcatcaggttaa |
| 8 | Base editor | A3B-dCasPhi-2 | atgggaagtcatcaccaccaccaccattcaggtgagaacctgtacttcca |
| | | | gagcatggctccgaaaaagaaacgtaaggtgggtatccacggcgttccgg |
| | | | cgatggaaatactaaggtatttaatggatcccgacactttcaccttcaat |
| | | | ttcaacaacgatccattggtcttgcgccgtcgtcagacctacttgtgcta |
| | | | tgaggttgaacgtctggataacggtacgtgggttttaatggatcagcata |
| | | | tgggtttcctgtgtaatgaagcgaagaacctgctgtgcggtttttacggc |
| | | | cgtcatgcggaactgcgttttcttgacctggttccgagcctgcaactgga |
| | | | cccggctcagatttaccgtgttacctggtttatctcgtggagcccgtgtt |
| | | | ttagctggggctgcgcgggtgaagtgcgcgcgtttttgcaagagaacacc |
| | | | cacgtgcgtctccgcatcttcgccgcacgtatttacgattatgacccgct |
| | | | gtataaagaggccctgcaaatgctgagagatgctggcgcacaagtgagca |
| | | | ttatgacctacgacgagttcgagtactgctgggatacgttcgtgtatcgc |
| | | | caaggttgcccgtttcagccgtgggacggcctggaggagcacagccaggc |
| | | | gctctccggtcgtttgcgcgcgatcctgcaatctggaagtgagacacccg |
| | | | ggacttcagaatccgcgaccccggagtctggaatgccaaaacccgctgta |
| | | | gagtcagaattttcaaaagttttgaaaaagcactttccgggagaacgttt |
| | | | ccgctcgagctatatgaaaagaggcggtaagatcctggctgcccaaggcg |
| | | | aagaggcagttgttgcgtatctacagggcaagtccgaggaggagcctccg |
| | | | aacttccagccaccggcgaagtgccacgtggtgaccaaatcccgtgactt |
| | | | cgcggaatggccgatcatgaaggcgtcggaagcgattcaacgttatatct |
| | | | atgcactgtcgaccaccgaacgcgcggcctgtaaaccgggcaaaagtagt |
| | | | gagagccatgcagcgtggttcgccgctaccggtgtcagcaaccacggcta |
| | | | cagccacgttcagggtctgaacctgatttttgatcataccttgggccgtt |
| | | | acgatggtgttctgaaaaaggtccaactgcgcaacgaaaaggcgagagcg |
| | | | cgtttagagagcattaacgcgtctagagcagatgaaggtttaccggagat |
| | | | taaagccgaggaggaagaagtggctacgaacgaaaccggccacctgttgc |
| | | | aaccgccgggtatcaatccgagcttttacgtgtatcagaccattagcccg |
| | | | caggcataccgtccgcgtgacgagatcgtgctgccgccggagtacgcagg |
| | | | ttacgtacgtgacccgaatgctccgattccgctgggtgtggtgcgtaacc |
| | | | gttgcgatatccaaaaagggtgcccgggctacatcccggaatggcagcgt |
| | | | gaagcaggtaccgcgatcagcccgaaaacaggcaaggccgtaactgtgcc |
| | | | gggcctgtctccgaagaaaaacaaacgtatgcgtaggtactggcgctctg |
| | | | agaaggagaaagctcaagatgcgctgttggttaccgtacgcattggtacc |
| | | | gattgggttgtcatcgacgtgcgcggtctgttgcgcaacgcccgttggcg |
| | | | caccatcgctcccaaggacatcagcctgaatgcgctcctcgatttgttta |
| | | | ccggtgatccggtgatcgatgtgcgtcgcaatattgttacctttacgtac |
| | | | accctcgacgcctgtggtacctacgcccgtaaatggaccttgaaaggcaa |
| | | | acagacgaaagcgaccctggacaaactgacggccactcagacggttgcgt |
| | | | tggttgcgattgcgcttggtcagaccaacccgatttccgctggcattagc |
| | | | cgtgttacacaagaaaatggtgcactgcaatgcgaaccgctggaccggtt |
| | | | caccctgccggatgacctgttgaaggacatcagcgcatatcgcattgctt |
| | | | gggacagaaacgaagaggaactacgcgctcgcagcgtcgaggccctgccg |
| | | | gaggcgcaacaagcggaggtccgtgcgctggacggggtgtccaaagaaac |
| | | | cgcgcgtacccaactgtgcgcagatttcggcctggacccgaaacgtctgc |
| | | | cgtgggataaaatgagctccaatactacctttatcagcgaagccttgctg |
| | | | tctaacagcgtgagccgtgatcaggtttttttcactccggctccgaagaa |
| | | | gggtgctaagaaaaaagcgcctgttgaagttatgcgtaaagaccgcactt |
| | | | gggcacgtgcgtacaagccgcgtctgagcgtcgaagctcagaaactgaag |
| | | | aacgaagccctgtgggcacttaagcgcacctccccggagtatttgaagct |
| | | | gagccgtcgcaaagaggagctgtgtcgtcgtagcatcaactatgttatcg |
| | | | aaaagacccgtcgccgtacgcaatgtcagattgtgatcccggtgatcgag |
| | | | gatctgaatgttcgcttctttcatggttctggtaagagactgcccggttg |
| | | | ggataatttcttcaccgctaaaaaagagaaccgttggttcatccagggcc |
| | | | tgcacaaagcgtttagcgatctgcgtacgcaccgttccttctatgttttt |
| | | | gaagttcgtccggagcgcacctctattacctgtccaaagtgcggtcattg |
| | | | cgaggtgggtaatcgtgacggtgaggcgtttcagtgcctgagctgcggca |
| | | | agacgtgcaacgcagatcttgacgtcgcgacccataatctgacccaagtg |
| | | | gcgttgaccggtaagaccatgccgaagcgcgaggagccgcgtgacgctca |
| | | | gggtacagcgccagcgcgcaagactaagaaggcgagcaaatcaaaggcgc |
| | | | caccggcggagcgcgaagaccagaccccggcgcaagagccaagccagacc |
| | | | agttctggaggtagcgggggtagcggtggtagcactaacttaagtgatat |
| | | | tatcgagaaagaaaccgggaaacagctggtgatccaagaaagcattctga |
| | | | tgctgcccgaagaagttgaagaagtgattggtaacaagcctgagtcggac |
| | | | atcttagtgcacaccgcgtacgatgaaagcacggatgagaatgtgatgct |
| | | | gctcacctctgatgcccccgaatacaagccgtgggctctggtgatccagg |
| | | | actctaacggagaaaacaaaattaagatgctctctggcggctccatggcg |
| | | | cctaaaaaaaaacgcaaagtggggattcacggggttccggcgtaa |
| 9 | Base editor | A3B-dLbCas12a | atgggaagtcatcaccaccaccaccattcaggtgagaacctgtacttcca |
| | | | gagcatggctccgaaaaagaaacgtaaggtgggtatccacggcgttccgg |
| | | | cgatggaaatactaaggtatttaatggatcccgacactttcaccttcaat |
| | | | ttcaacaacgatccattggtcttgcgccgtcgtcagacctacttgtgcta |
| | | | tgaggttgaacgtctggataacggtacgtgggttttaatggatcagcata |
| | | | tgggtttcctgtgtaatgaagcgaagaacctgctgtgcggtttttacggc |
| | | | cgtcatgcggaactgcgttttcttgacctggttccgagcctgcaactgga |
| | | | cccggctcagatttaccgtgttacctggtttatctcgtggagcccgtgtt |
| | | | ttagctggggctgcgcgggtgaagtgcgcgcgtttttgcaagagaacacc |
| | | | cacgtgcgtctccgcatcttcgccgcacgtatttacgattatgacccgct |
| | | | gtataaagaggccctgcaaatgctgagagatgctggcgcacaagtgagca |
| | | | ttatgacctacgacgagttcgagtactgctgggatacgttcgtgtatcgc |
| | | | caaggttgcccgtttcagccgtgggacggcctggaggagcacagccaggc |
| | | | gctctccggtcgtttgcgcgcgatcctgcaatctggaagtgagacacccg |
| | | | ggacttcagaatccgcgaccccggagtctggaatgtcaaaactagaaaag |
| | | | tttacaaattgttactccctgtcgaaaaccctgagattcaaagcgatccc |
| | | | ggtgggtaaaactcaagagaacattgataacaaacgtctgctggttgagg |
| | | | acgaaaagcgcgcggaggattacaaaggcgttaaaaagctgttggaccgt |
| | | | tattacctgtcctttattaatgatgttctgcacagcattaaattaaagaa |
| | | | cctaaacaattacatctccctcttccgtaaaaagacccgcaccgaaaagg |
| | | | aaaataaagaactggagaaccttgagattaatctgcgtaaagaaatcgct |
| | | | aaagcgtttaaaggcaacgagggttataaatccttatttaaaaaagacat |
| | | | catcgaaacgattctcccagaattcctggacgacaaagacgagatcgcgc |
| | | | tggttaattccttcaacggctttaccaccgcattcaccggcttttttgat |
| | | | aaccgcgaaaacatgttctctgaagaggccaaaagcacctcaattgcgtt |
| | | | tcgctgcattaacgaaaatctgacgcgttatatctctaacatggatatct |
| | | | tcgagaaggttgacgctatctttgataaacatgaagtccaggagatcaag |
| | | | gagaagatcctgaacagcgactacgacgtggaggatttcttcgagggtga |
| | | | gttcttcaattttgttttgacccaagaaggcatcgatgtctacaatgcga |
| | | | taattggtggttttgttaccgagagcggggagaaaattaagggtctgaac |
| | | | gaatatatcaacttgtacaaccagaaaaccaaacaaaaactgccgaagtt |
| | | | taagccgctgtacaagcaagtcttgtcggatcgtgaatcgctgagctttt |
| | | | acggcgaaggctacaccagcgatgaggaggtgttggaggtgttccgcaat |
| | | | acgttgaataagaactctgaaattttttccagcattaagaagctggaaaa |
| | | | actattcaagaacttcgacgagtatagcagcgcaggtatctttgtgaaaa |
| | | | acggtccggctattagcaccatctcgaaagacattttcggcgaatggaat |
| | | | gttattcgtgataagtggaatgccgaatacgacgacattcatctgaagaa |
| | | | aaaggcggttgtaactgaaaagtacgaggacgatcgtcgtaagtcgttca |
| | | | aaaagatcggctccttctctctggagcaactgcaagagtacgccgatgcc |
| | | | gatttgtcagtcgtggagaagctgaaagagatcatcattcaaaaagttga |
| | | | cgaaatctataaggtctatggtagcagtgaaaaactgtttgacgcggact |
| | | | tcgtgctggagaaaagcctgaagaaaaatgacgccgtggtggcaatcatg |
| | | | aaagatcttctggatagcgtcaagtctttcgaaaactacatcaaagcgtt |
| | | | tttcggtgaaggcaaggaaacgaatcgtgacgagagcttctatggtgatt |
| | | | tcgtcctagcctatgatatcctgcttaaggtggatcacatctatgatgcc |
| | | | atccgcaactatgttacccagaaaccgtactcaaaggacaagttcaagtt |
| | | | atacttccagaatccgcagtttatgggtggctgggataaagataaggaaa |
| | | | ccgactatcgcgccaccattttacgttacggtagtaagtactatctggca |
| | | | atcatggataaaaaatatgcgaaatgcctgcaaaagatcgacaaggacga |
| | | | tgttaacggcaattatgagaagatcaactacaaactgctgccgggcccaa |
| | | | ataagatgctgccgaaggtgtttttcagcaaaaaatggatggcgtattac |
| | | | aatccgagcgaggacatccaaaagatctacaaaaacggtacgtttaagaa |
| | | | gggtgatatgttcaacctcaacgattgtcataaacttattgacttcttca |
| | | | aggatagcatatctcgttatccgaagtggagcaacgcgtatgactttaac |
| | | | tttagcgaaaccgagaaatacaaagatatcgcgggtttctatcgtgaagt |
| | | | tgaagaacagggttacaaagtgtccttcgagtccgcgagcaagaaggagg |
| | | | tagacaagctggtcgaggagggtaagttgtacatgtttcagatttacaat |
| | | | aaggacttttccgacaagtcccatggtacaccgaatttgcacaccatgta |
| | | | tttcaagcttctgttcgacgagaacaaccacggccagatccgtctgagcg |
| | | | gtggtgcagagctgttcatgcgtagagctagcctgaagaaagaagagttg |
| | | | gtcgttcatccggctaattcgccgatcgcgaacaaaaatccagataaccc |
| | | | taaaaagactacgaccctgagctatgatgtgtataaagacaaacgcttta |
| | | | gcgaagaccagtatgagttacacattccgatcgctatcaacaagtgcccg |
| | | | aagaatattttcaagatcaacaccgaagtgcgtgttcttttgaagcacga |
| | | | tgacaacccgtatgtgattggtattgctcgtggtgaacgtaatctgctgt |
| | | | acattgtggttgtggacggcaaaggaaacatcgtggaacagtacagcctg |
| | | | aacgaaatcattaacaatttcaacggcattcgcattaagacggattacca |
| | | | ttctttgctggacaaaaaggagaaagagcgtttcgaggcccgtcagaact |
| | | | ggacctccatcgagaacattaaggagctgaaggcgggttacatcagccag |
| | | | gtggtgcacaaaatctgcgaactggtggagaagtacgacgcggtgattgc |
| | | | tttggcggatttgaactccggttttaagaacagccgcgttaaagtggaaa |
| | | | agcaggtttatcagaaattcgaaaaaatgctgattgataaattgaattac |
| | | | atggttgacaaaaagtctaacccgtgtgcaaccggcggtgcgctgaaggg |
| | | | ctaccaaatcaccaataagtttgaaagctttaagagcatgagcacgcaaa |
| | | | atggcttcattttttacattccggcgtggctgaccagcaagatcgatccg |
| | | | tcaaccggctttgttaatttactgaagacgaagtatacctctattgccga |
| | | | ctctaaaaaatttattagttccttcgatagaatcatgtatgttccggaag |
| | | | aggacctgttcgaatttgcactggactacaagaactttagccgtaccgat |
| | | | gctgattatataaagaaatggaaactctacagctatggcaaccgcatccg |
| | | | tatctttcgcaacccgaaaaaaaacaacgtgttcgactgggaagaggtat |
| | | | gtctgacttcagcgtacaaggagcttttcaataaatatggtatcaactac |
| | | | cagcagggtgatatccgtgcactgctttgcgagcaaagcgataaagcgtt |
| | | | ctattcaagcttcatggcgctgatgagcctgatgctgcaaatgcgcaaca |
| | | | gcatcaccggtcgtaccgatgttgcatttctgatttctccagtaaaaaac |
| | | | agcgatggtatcttttacgacagccgtaattacgaggcccaagaaaatgc |
| | | | gattttgccgaagaacgctgacgcgaatggtgcatacaacattgcaagga |
| | | | aagttctgtgggcaatcggccaatttaaaaaagcggaagatgagaagctg |
| | | | gacaaagtgaaaattgcgattagcaacaaggagtggctggagtatgcgca |
| | | | gacctccgttaaatcagggggaagtggcggaagcggtgggtctacgaacc |
| | | | tgagcgacattattgaaaaagaaactggtaaacagttggtgatccaagaa |
| | | | tccattctgatgttaccggaggaagtggaagaggtgatcggcaacaagcc |
| | | | ggagtccgacatcctggttcacaccgcgtatgatgagtcgaccgatgaga |
| | | | atgttatgctgctgaccagcgacgccccagagtacaaaccgtgggcgctc |
| | | | gtcatccaggatagcaatggcgaaaacaagatcaagatgttgtctggcgg |
| | | | tagcatggctccgaaaaagaagcgtaaagtaggtattcatggtgttccgg |
| | | | catcaggttaa |
| 10 | Base editor | AID-dCas9 | atgggaagtcatcaccaccaccaccattcaggtgagaacctgtacttcca |
| | | | gagcatggctccgaaaaagaaacgtaaggtgggtatccacggcgttccgg |
| | | | cgatggattcactattaatgaataggagaaaattcctgtatcagtttaag |
| | | | aacgtccgttgggctaaaggtcgtcgtgagacgtatttatgctacgtggt |
| | | | gaaacgtcgcgacagcgcaacctccttcagcctggacttcggctatctgc |
| | | | gtaacaaaaacggctgccacgtggagctgctgtttttgcgctacattagc |
| | | | gactgggatttggacccaggtcgctgctatcgtgttacctggtttacgag |
| | | | ctggtcgccgtgttacgactgtgcgcgtcatgtcgcggattttctgcgcg |
| | | | gtaatccgaatctgtccctgcgtatttttaccgcacgtctctacttctgc |
| | | | gaagatcgtaaggcggaaccggaaggcttgcgtcgcctgcatagagcggg |
| | | | tgttcagatcgctatcatgaccttcaaagattatttctactgctggaata |
| | | | ccttcgtggagaaccacgagcgtactttcaaggcctgggaaggccttcac |
| | | | gaaaacagcgttcgcttgtctcgtcaactgcgccgcatcctgctgccgct |
| | | | gtacgaggttgatgatctcagagacgcgtttcgtaccctgggtttgtctg |
| | | | gaagtgagacacccgggacttcagaatccgcgaccccggagtctggaatg |
| | | | gataaaaagtattcaataggactagctattggcaccaacagcgttggttg |
| | | | ggcagttatcaccgatgaatataaggtcccgagcaagaagtttaaggtcc |
| | | | tgggcaacaccgatcgccacagcataaaaaagaaccttattggtgcgctg |
| | | | ctgttcgattctggtgagactgccgaagcgacccgtctgaagagaaccgc |
| | | | gcgtcgtagatacacccggcgcaagaaccgtatctgctatctgcaagaaa |
| | | | ttttcagcaacgagatggctaaggtcgacgactccttctttcatcgcctg |
| | | | gaggaaagctttctggttgaggaagacaaaaagcacgagcgtcatccgat |
| | | | tttcggtaacattgttgatgaagttgcctaccacgaaaaataccccacaa |
| | | | tttatcatctgcgtaaaaaactggtagactccaccgataaggcggattta |
| | | | cgccttatctatttggcgctggcgcatatgattaagttccgcggtcattt |
| | | | tttgatcgagggcgacctgaatccggataacagcgacgttgacaaactgt |
| | | | ttatccagctggtgcagacctataaccagttgttcgaagagaacccgatt |
| | | | aatgcgtctggcgtggacgcgaaggcgatcctgagcgctcgtctgtccaa |
| | | | gtcacgccgcctggagaacttgatcgcgcaactgccgggtgagaagaaaa |
| | | | acggcttgttcggcaatctgattgctctgtccctaggtttgaccccgaat |
| | | | ttcaaatctaattttgatctggcggaagatgcgaaattacagttgagcaa |
| | | | ggacacttacgatgacgatctggacaacctgctcgctcagatcggcgatc |
| | | | agtatgccgatcttttcctggccgcgaagaacctgagcgatgccatcttg |
| | | | ctatcggacatcttacgtgttaacaccgaaatcaccaaagctccgctaag |
| | | | cgcgtccatgattaaacgttacgatgaacaccaccaggatctgacgctgc |
| | | | tgaaagcgttggtgcgccagcaactgccggaaaaatacaaggaaattttc |
| | | | ttcgaccaaagcaagaacggttatgcaggctatatcgacggtggtgcgtc |
| | | | gcaagaagaattttataaattcatcaaaccgattttggagaagatggatg |
| | | | gtaccgaggagctgctggtaaaattgaatcgggaagacctcttgcgaaag |
| | | | cagcgtactttcgataatggtagcatcccgcaccagattcatctgggcga |
| | | | acttcacgcaatcctacgtcgtcaagaggatttctacccgtttctgaagg |
| | | | ataaccgtgaaaagatcgagaagattctgacctttcgtattccgtattat |
| | | | gtgggtccgctggcgagaggcaacagccgttttgcgtggatgacccgcaa |
| | | | gtcggaagagactatcacgccgtggaatttcgaggaggtcgtggacaagg |
| | | | gtgcatcagcgcaatcttttattgaacgtatgaccaattttgataaaaac |
| | | | cttccaaatgaaaaagtgttgccgaagcacagcttgctgtacgagtattt |
| | | | caccgtttacaatgaactgacgaaagtcaagtacgttaccgaaggtatgc |
| | | | gtaagccagcctttctgtctggggagcaaaaaaaagccattgtggatctg |
| | | | ctgttcaagaccaatcgcaaggttaccgtcaagcaactgaaagaggatta |
| | | | cttcaaaaaaatcgaatgttttgactccgtggaaatcagcggcgttgaag |
| | | | accgctttaatgcatctctgggcacctaccacgatctactcaagatcatc |
| | | | aaggataaggacttcctggacaacgaagaaaatgaggatatcttggaaga |
| | | | tattgtgctgactttaacgttgttcgaggatcgtgagatgattgaagaac |
| | | | gcctcaaaacgtacgcacatctgtttgacgataaggtgatgaaacagcta |
| | | | aagcgccgtcgttacaccggttggggtcgtctgtctagaaagctgattaa |
| | | | cggcattcgtgacaaacaatctggcaagaccatcctggacttcctcaaat |
| | | | ccgacggcttcgcaaatcgtaatttcatgcaactgatccatgatgatagc |
| | | | ctgacctttaaagaagatattcagaaggctcaagtgtccggccagggtga |
| | | | ctcgctgcatgagcatatcgcgaatctggctggctcccctgcgataaaaa |
| | | | aaggtattctccaaacggtgaaagttgtggatgaactggttaaagtcatg |
| | | | ggtcgtcataaaccggaaaacatcgtgattgagatggcacgtgagaacca |
| | | | gaccacccagaaaggtcaaaaaaacagtcgtgagcgtatgaaacgtatcg |
| | | | aagaggggattaaggagttaggcagccagattctgaaggagcacccggtt |
| | | | gagaacacccagctgcaaaacgagaagctctacctgtattacctgcaaaa |
| | | | cggccgtgacatgtatgttgaccaggagctggacatcaatcgtctcagcg |
| | | | actacgacgtggatgctatcgtgccgcagagctttctaaaagatgattcc |
| | | | atcgacaataaagtgctcacccgtagcgacaagaaccgtggtaaaagcga |
| | | | caacgtgccgagcgaggaagtagttaagaaaatgaaaaactactggcgtc |
| | | | agctattgaacgccaagcttatcacccagcgtaagtttgacaacttgacg |
| | | | aaggcagagcgtggcggcctgagcgagctagacaaggcgggttttattaa |
| | | | acgccagctggtggaaactcgccagattaccaaacacgttgcgcaaatct |
| | | | tggactcacgcatgaatacgaagtacgacgaaaacgataaattgatccgc |
| | | | gaggtgaaggtcataaccctgaaatctaaactggttagcgatttccggaa |
| | | | ggactttcaattttataaagtccgggagattaacaactatcaccatgcac |
| | | | acgacgcttacttaaatgcggttgttggcacggccctgatcaagaagtac |
| | | | ccgaaactggaaagcgagtttgtttatggtgactacaaggtgtatgacgt |
| | | | tcgcaagatgattgcaaaatccgaacaagagattggtaaggcgacggcta |
| | | | agtattttttctacagcaatatcatgaacttcttcaagactgaaatcacc |
| | | | ctggcgaacggtgaaattcgtaaacgtccgttgatcgaaaccaacggtga |
| | | | aaccggtgaaatcgtttgggataaaggtagggattttgcgaccgttcgta |
| | | | aggtcctgagcatgccgcaggttaacatcgtgaaaaagaccgaagtgcag |
| | | | accggcggtttctccaaggagagtatcttaccgaagcgcaacagtgacaa |
| | | | gctgatcgcgcgtaaaaaagactgggatccgaaaaaatacggcggctttg |
| | | | actccccgaccgtcgcgtacagcgtactggtcgtggctaaagtggagaaa |
| | | | ggcaaatcgaaaaagctgaaatccgttaaagagttgctgggtattacgat |
| | | | tatggaacgttcgagcttcgagaaaaatccgatcgacttcctggaggcca |
| | | | aaggctacaaggaagttaaaaaagacctgatcattaaactgccgaagtac |
| | | | agcctgttcgagctcgagaacggtcgcaaacgcatgctggcgagcgcggg |
| | | | tgagctccaaaaaggcaacgaattggctctgccgagcaagtacgtgaact |
| | | | tcctgtatctggcctcccattatgaaaaactgaagggtagcccggaagat |
| | | | aacgaacaaaagcagctgttcgtggaacaacataaacactatctggacga |
| | | | gattattgagcaaatcagcgagttcagcaaacgcgtgattctggcggatg |
| | | | ctaatctggacaaggttctttcagcctataataaacaccgtgataagccg |
| | | | atccgtgaacaggcagaaaatattattcacctgttcaccctgacgaacct |
| | | | gggtgcgccagcagcgtttaaatacttcgacacaacgatcgaccggaagc |
| | | | gttataccagcaccaaagaggttcttgacgccaccttgattcaccaaagc |
| | | | atcacgggtctgtatgaaacccgtattgacttgagccagttgggcggaga |
| | | | ttcagggggaagtggcggaagcggtgggtctacgaacctgagcgacatta |
| | | | ttgaaaaagaaactggtaaacagttggtgatccaagaatccattctgatg |
| | | | ttaccggaggaagtggaagaggtgatcggcaacaagccggagtccgacat |
| | | | cctggttcacaccgcgtatgatgagtcgaccgatgagaatgttatgctgc |
| | | | tgaccagcgacgccccagagtacaaaccgtgggcgctcgtcatccaggat |
| | | | agcaatggcgaaaacaagatcaagatgttgtctggcggtagcatggctcc |
| | | | gaaaaagaagcgtaaagtaggtattcatggtgttccggcatcaggttaa |
| 11 | Base editor | AID-dCasPhi-2 | atgggaagtcatcaccaccaccaccattcaggtgagaacctgtacttcca |
| | | | gagcatggctccgaaaaagaaacgtaaggtgggtatccacggcgttccgg |
| | | | cgatggattcactattaatgaataggagaaaattcctgtatcagtttaag |
| | | | aacgtccgttgggctaaaggtcgtcgtgagacgtatttatgctacgtggt |
| | | | gaaacgtcgcgacagcgcaacctccttcagcctggacttcggctatctgc |
| | | | gtaacaaaaacggctgccacgtggagctgctgtttttgcgctacattagc |
| | | | gactgggatttggacccaggtcgctgctatcgtgttacctggtttacgag |
| | | | ctggtcgccgtgttacgactgtgcgcgtcatgtcgcggattttctgcgcg |
| | | | gtaatccgaatctgtccctgcgtatttttaccgcacgtctctacttctgc |
| | | | gaagatcgtaaggcggaaccggaaggcttgcgtcgcctgcatagagcggg |
| | | | tgttcagatcgctatcatgaccttcaaagattatttctactgctggaata |
| | | | ccttcgtggagaaccacgagcgtactttcaaggcctgggaaggccttcac |
| | | | gaaaacagcgttcgcttgtctcgtcaactgcgccgcatcctgctgccgct |
| | | | gtacgaggttgatgatctcagagacgcgtttcgtaccctgggtttgtctg |
| | | | gaagtgagacacccgggacttcagaatccgcgaccccggagtctggaatg |
| | | | ccaaaacccgctgtagagtcagaattttcaaaagttttgaaaaagcactt |
| | | | tccgggagaacgtttccgctcgagctatatgaaaagaggcggtaagatcc |
| | | | tggctgcccaaggcgaagaggcagttgttgcgtatctacagggcaagtcc |
| | | | gaggaggagcctccgaacttccagccaccggcgaagtgccacgtggtgac |
| | | | caaatcccgtgacttcgcggaatggccgatcatgaaggcgtcggaagcga |
| | | | ttcaacgttatatctatgcactgtcgaccaccgaacgcgcggcctgtaaa |
| | | | ccgggcaaaagtagtgagagccatgcagcgtggttcgccgctaccggtgt |
| | | | cagcaaccacggctacagccacgttcagggtctgaacctgatttttgatc |
| | | | ataccttgggccgttacgatggtgttctgaaaaaggtccaactgcgcaac |
| | | | gaaaaggcgagagcgcgtttagagagcattaacgcgtctagagcagatga |
| | | | aggtttaccggagattaaagccgaggaggaagaagtggctacgaacgaaa |
| | | | ccggccacctgttgcaaccgccgggtatcaatccgagcttttacgtgtat |
| | | | cagaccattagcccgcaggcataccgtccgcgtgacgagatcgtgctgcc |
| | | | gccggagtacgcaggttacgtacgtgacccgaatgctccgattccgctgg |
| | | | gtgtggtgcgtaaccgttgcgatatccaaaaagggtgcccgggctacatc |
| | | | ccggaatggcagcgtgaagcaggtaccgcgatcagcccgaaaacaggcaa |
| | | | ggccgtaactgtgccgggcctgtctccgaagaaaaacaaacgtatgcgta |
| | | | ggtactggcgctctgagaaggagaaagctcaagatgcgctgttggttacc |
| | | | gtacgcattggtaccgattgggttgtcatcgacgtgcgcggtctgttgcg |
| | | | caacgcccgttggcgcaccatcgctcccaaggacatcagcctgaatgcgc |
| | | | tcctcgatttgtttaccggtgatccggtgatcgatgtgcgtcgcaatatt |
| | | | gttacctttacgtacaccctcgacgcctgtggtacctacgcccgtaaatg |
| | | | gaccttgaaaggcaaacagacgaaagcgaccctggacaaactgacggcca |
| | | | ctcagacggttgcgttggttgcgattgcgcttggtcagaccaacccgatt |
| | | | tccgctggcattagccgtgttacacaagaaaatggtgcactgcaatgcga |
| | | | accgctggaccggttcaccctgccggatgacctgttgaaggacatcagcg |
| | | | catatcgcattgcttgggacagaaacgaagaggaactacgcgctcgcagc |
| | | | gtcgaggccctgccggaggcgcaacaagcggaggtccgtgcgctggacgg |
| | | | ggtgtccaaagaaaccgcgcgtacccaactgtgcgcagatttcggcctgg |
| | | | acccgaaacgtctgccgtgggataaaatgagctccaatactacctttatc |
| | | | agcgaagccttgctgtctaacagcgtgagccgtgatcaggtttttttcac |
| | | | tccggctccgaagaagggtgctaagaaaaaagcgcctgttgaagttatgc |
| | | | gtaaagaccgcacttgggcacgtgcgtacaagccgcgtctgagcgtcgaa |
| | | | gctcagaaactgaagaacgaagccctgtgggcacttaagcgcacctcccc |
| | | | ggagtatttgaagctgagccgtcgcaaagaggagctgtgtcgtcgtagca |
| | | | tcaactatgttatcgaaaagacccgtcgccgtacgcaatgtcagattgtg |
| | | | atcccggtgatcgaggatctgaatgttcgcttctttcatggttctggtaa |
| | | | gagactgcccggttgggataatttcttcaccgctaaaaaagagaaccgtt |
| | | | ggttcatccagggcctgcacaaagcgtttagcgatctgcgtacgcaccgt |
| | | | tccttctatgtttttgaagttcgtccggagcgcacctctattacctgtcc |
| | | | aaagtgcggtcattgcgaggtgggtaatcgtgacggtgaggcgtttcagt |
| | | | gcctgagctgcggcaagacgtgcaacgcagatcttgacgtcgcgacccat |
| | | | aatctgacccaagtggcgttgaccggtaagaccatgccgaagcgcgagga |
| | | | gccgcgtgacgctcagggtacagcgccagcgcgcaagactaagaaggcga |
| | | | gcaaatcaaaggcgccaccggcggagcgcgaagaccagaccccggcgcaa |
| | | | gagccaagccagaccagttctggaggtagcgggggtagcggtggtagcac |
| | | | taacttaagtgatattatcgagaaagaaaccgggaaacagctggtgatcc |
| | | | aagaaagcattctgatgctgcccgaagaagttgaagaagtgattggtaac |
| | | | aagcctgagtcggacatcttagtgcacaccgcgtacgatgaaagcacgga |
| | | | tgagaatgtgatgctgctcacctctgatgcccccgaatacaagccgtggg |
| | | | ctctggtgatccaggactctaacggagaaaacaaaattaagatgctctct |
| | | | ggcggctccatggcgcctaaaaaaaaacgcaaagtggggattcacggggt |
| | | | tccggcgtaa |
| 12 | Base editor | AID-dLbCas12a | atgggaagtcatcaccaccaccaccattcaggtgagaacctgtacttcca |
| | | | gagcatggctccgaaaaagaaacgtaaggtgggtatccacggcgttccgg |
| | | | cgatggattcactattaatgaataggagaaaattcctgtatcagtttaag |
| | | | aacgtccgttgggctaaaggtcgtcgtgagacgtatttatgctacgtggt |
| | | | gaaacgtcgcgacagcgcaacctccttcagcctggacttcggctatctgc |
| | | | gtaacaaaaacggctgccacgtggagctgctgtttttgcgctacattagc |
| | | | gactgggatttggacccaggtcgctgctatcgtgttacctggtttacgag |
| | | | ctggtcgccgtgttacgactgtgcgcgtcatgtcgcggattttctgcgcg |
| | | | gtaatccgaatctgtccctgcgtatttttaccgcacgtctctacttctgc |
| | | | gaagatcgtaaggcggaaccggaaggcttgcgtcgcctgcatagagcggg |
| | | | tgttcagatcgctatcatgaccttcaaagattatttctactgctggaata |
| | | | ccttcgtggagaaccacgagcgtactttcaaggcctgggaaggccttcac |
| | | | gaaaacagcgttcgcttgtctcgtcaactgcgccgcatcctgctgccgct |
| | | | gtacgaggttgatgatctcagagacgcgtttcgtaccctgggtttgtctg |
| | | | gaagtgagacacccgggacttcagaatccgcgaccccggagtctggaatg |
| | | | tcaaaactagaaaagtttacaaattgttactccctgtcgaaaaccctgag |
| | | | attcaaagcgatcccggtgggtaaaactcaagagaacattgataacaaac |
| | | | gtctgctggttgaggacgaaaagcgcgcggaggattacaaaggcgttaaa |
| | | | aagctgttggaccgttattacctgtcctttattaatgatgttctgcacag |
| | | | cattaaattaaagaacctaaacaattacatctccctcttccgtaaaaaga |
| | | | cccgcaccgaaaaggaaaataaagaactggagaaccttgagattaatctg |
| | | | cgtaaagaaatcgctaaagcgtttaaaggcaacgagggttataaatcctt |
| | | | atttaaaaaagacatcatcgaaacgattctcccagaattcctggacgaca |
| | | | aagacgagatcgcgctggttaattccttcaacggctttaccaccgcattc |
| | | | accggcttttttgataaccgcgaaaacatgttctctgaagaggccaaaag |
| | | | cacctcaattgcgtttcgctgcattaacgaaaatctgacgcgttatatct |
| | | | ctaacatggatatcttcgagaaggttgacgctatctttgataaacatgaa |
| | | | gtccaggagatcaaggagaagatcctgaacagcgactacgacgtggagga |
| | | | tttcttcgagggtgagttcttcaattttgttttgacccaagaaggcatcg |
| | | | atgtctacaatgcgataattggtggttttgttaccgagagcggggagaaa |
| | | | attaagggtctgaacgaatatatcaacttgtacaaccagaaaaccaaaca |
| | | | aaaactgccgaagtttaagccgctgtacaagcaagtcttgtcggatcgtg |
| | | | aatcgctgagcttttacggcgaaggctacaccagcgatgaggaggtgttg |
| | | | gaggtgttccgcaatacgttgaataagaactctgaaattttttccagcat |
| | | | taagaagctggaaaaactattcaagaacttcgacgagtatagcagcgcag |
| | | | gtatctttgtgaaaaacggtccggctattagcaccatctcgaaagacatt |
| | | | ttcggcgaatggaatgttattcgtgataagtggaatgccgaatacgacga |
| | | | cattcatctgaagaaaaaggcggttgtaactgaaaagtacgaggacgatc |
| | | | gtcgtaagtcgttcaaaaagatcggctccttctctctggagcaactgcaa |
| | | | gagtacgccgatgccgatttgtcagtcgtggagaagctgaaagagatcat |
| | | | cattcaaaaagttgacgaaatctataaggtctatggtagcagtgaaaaac |
| | | | tgtttgacgcggacttcgtgctggagaaaagcctgaagaaaaatgacgcc |
| | | | gtggtggcaatcatgaaagatcttctggatagcgtcaagtctttcgaaaa |
| | | | ctacatcaaagcgtttttcggtgaaggcaaggaaacgaatcgtgacgaga |
| | | | gcttctatggtgatttcgtcctagcctatgatatcctgcttaaggtggat |
| | | | cacatctatgatgccatccgcaactatgttacccagaaaccgtactcaaa |
| | | | ggacaagttcaagttatacttccagaatccgcagtttatgggtggctggg |
| | | | ataaagataaggaaaccgactatcgcgccaccattttacgttacggtagt |
| | | | aagtactatctggcaatcatggataaaaaatatgcgaaatgcctgcaaaa |
| | | | gatcgacaaggacgatgttaacggcaattatgagaagatcaactacaaac |
| | | | tgctgccgggcccaaataagatgctgccgaaggtgtttttcagcaaaaaa |
| | | | tggatggcgtattacaatccgagcgaggacatccaaaagatctacaaaaa |
| | | | cggtacgtttaagaagggtgatatgttcaacctcaacgattgtcataaac |
| | | | ttattgacttcttcaaggatagcatatctcgttatccgaagtggagcaac |
| | | | gcgtatgactttaactttagcgaaaccgagaaatacaaagatatcgcggg |
| | | | tttctatcgtgaagttgaagaacagggttacaaagtgtccttcgagtccg |
| | | | cgagcaagaaggaggtagacaagctggtcgaggagggtaagttgtacatg |
| | | | tttcagatttacaataaggacttttccgacaagtcccatggtacaccgaa |
| | | | tttgcacaccatgtatttcaagcttctgttcgacgagaacaaccacggcc |
| | | | agatccgtctgagcggtggtgcagagctgttcatgcgtagagctagcctg |
| | | | aagaaagaagagttggtcgttcatccggctaattcgccgatcgcgaacaa |
| | | | aaatccagataaccctaaaaagactacgaccctgagctatgatgtgtata |
| | | | aagacaaacgctttagcgaagaccagtatgagttacacattccgatcgct |
| | | | atcaacaagtgcccgaagaatattttcaagatcaacaccgaagtgcgtgt |
| | | | tcttttgaagcacgatgacaacccgtatgtgattggtattgctcgtggtg |
| | | | aacgtaatctgctgtacattgtggttgtggacggcaaaggaaacatcgtg |
| | | | gaacagtacagcctgaacgaaatcattaacaatttcaacggcattcgcat |
| | | | taagacggattaccattctttgctggacaaaaaggagaaagagcgtttcg |
| | | | aggcccgtcagaactggacctccatcgagaacattaaggagctgaaggcg |
| | | | ggttacatcagccaggtggtgcacaaaatctgcgaactggtggagaagta |
| | | | cgacgcggtgattgctttggcggatttgaactccggttttaagaacagcc |
| | | | gcgttaaagtggaaaagcaggtttatcagaaattcgaaaaaatgctgatt |
| | | | gataaattgaattacatggttgacaaaaagtctaacccgtgtgcaaccgg |
| | | | cggtgcgctgaagggctaccaaatcaccaataagtttgaaagctttaaga |
| | | | gcatgagcacgcaaaatggcttcattttttacattccggcgtggctgacc |
| | | | agcaagatcgatccgtcaaccggctttgttaatttactgaagacgaagta |
| | | | tacctctattgccgactctaaaaaatttattagttccttcgatagaatca |
| | | | tgtatgttccggaagaggacctgttcgaatttgcactggactacaagaac |
| | | | tttagccgtaccgatgctgattatataaagaaatggaaactctacagcta |
| | | | tggcaaccgcatccgtatctttcgcaacccgaaaaaaaacaacgtgttcg |
| | | | actgggaagaggtatgtctgacttcagcgtacaaggagcttttcaataaa |
| | | | tatggtatcaactaccagcagggtgatatccgtgcactgctttgcgagca |
| | | | aagcgataaagcgttctattcaagcttcatggcgctgatgagcctgatgc |
| | | | tgcaaatgcgcaacagcatcaccggtcgtaccgatgttgcatttctgatt |
| | | | tctccagtaaaaaacagcgatggtatcttttacgacagccgtaattacga |
| | | | ggcccaagaaaatgcgattttgccgaagaacgctgacgcgaatggtgcat |
| | | | acaacattgcaaggaaagttctgtgggcaatcggccaatttaaaaaagcg |
| | | | gaagatgagaagctggacaaagtgaaaattgcgattagcaacaaggagtg |
| | | | gctggagtatgcgcagacctccgttaaatcagggggaagtggcggaagcg |
| | | | gtgggtctacgaacctgagcgacattattgaaaaagaaactggtaaacag |
| | | | ttggtgatccaagaatccattctgatgttaccggaggaagtggaagaggt |
| | | | gatcggcaacaagccggagtccgacatcctggttcacaccgcgtatgatg |
| | | | agtcgaccgatgagaatgttatgctgctgaccagcgacgccccagagtac |
| | | | aaaccgtgggcgctcgtcatccaggatagcaatggcgaaaacaagatcaa |
| | | | gatgttgtctggcggtagcatggctccgaaaaagaagcgtaaagtaggta |
| | | | ttcatggtgttccggcatcaggttaa |
| 13 | Base editor | AmA1-dCas9 | atgggaagtcatcaccaccaccaccattcaggtgagaacctgtacttcca |
| | | | gagcatggctccgaaaaagaaacgtaaggtgggtatccacggcgttccgg |
| | | | cgatggctgattcaagtgaaaaaatgaggggacaatatatcagccgtgac |
| | | | acctttgagaaaaactacaaaccgattgacggcacgaaagaggcgcatct |
| | | | gctgtgcgaaattaaatggggtaagtacggcaagccgtggctgcattggt |
| | | | gtcaaaaccaacgtatgaatatccatgctgaagactacttcatgaacaac |
| | | | atcttcaaagccaaaaagcatccggtgcactgctacgtgacctggtattt |
| | | | gtcctggtcaccgtgcgcggattgtgcgagcaagatcgttaagttcctgg |
| | | | aggagcgtccttatctgaaactgaccatttatgttgcacagctgtattat |
| | | | cacactgaagaggagaaccgtaaaggtctgcgtttgctgcgttcgaagaa |
| | | | ggtgattatccgcgtgatggatatctctgattacaactactgctggaaag |
| | | | tctttgttagcaatcagaacggtaatgaagactactggccgctgcaattc |
| | | | gatccgtgggttaaggagaactacagccgcctcttggacattttctggga |
| | | | aagcaagtgccgttccccaaatccgtggtctggaagtgagacacccggga |
| | | | cttcagaatccgcgaccccggagtctggaatggataaaaagtattcaata |
| | | | ggactagctattggcaccaacagcgttggttgggcagttatcaccgatga |
| | | | atataaggtcccgagcaagaagtttaaggtcctgggcaacaccgatcgcc |
| | | | acagcataaaaaagaaccttattggtgcgctgctgttcgattctggtgag |
| | | | actgccgaagcgacccgtctgaagagaaccgcgcgtcgtagatacacccg |
| | | | gcgcaagaaccgtatctgctatctgcaagaaattttcagcaacgagatgg |
| | | | ctaaggtcgacgactccttctttcatcgcctggaggaaagctttctggtt |
| | | | gaggaagacaaaaagcacgagcgtcatccgattttcggtaacattgttga |
| | | | tgaagttgcctaccacgaaaaataccccacaatttatcatctgcgtaaaa |
| | | | aactggtagactccaccgataaggcggatttacgccttatctatttggcg |
| | | | ctggcgcatatgattaagttccgcggtcattttttgatcgagggcgacct |
| | | | gaatccggataacagcgacgttgacaaactgtttatccagctggtgcaga |
| | | | cctataaccagttgttcgaagagaacccgattaatgcgtctggcgtggac |
| | | | gcgaaggcgatcctgagcgctcgtctgtccaagtcacgccgcctggagaa |
| | | | cttgatcgcgcaactgccgggtgagaagaaaaacggcttgttcggcaatc |
| | | | tgattgctctgtccctaggtttgaccccgaatttcaaatctaattttgat |
| | | | ctggcggaagatgcgaaattacagttgagcaaggacacttacgatgacga |
| | | | tctggacaacctgctcgctcagatcggcgatcagtatgccgatcttttcc |
| | | | tggccgcgaagaacctgagcgatgccatcttgctatcggacatcttacgt |
| | | | gttaacaccgaaatcaccaaagctccgctaagcgcgtccatgattaaacg |
| | | | ttacgatgaacaccaccaggatctgacgctgctgaaagcgttggtgcgcc |
| | | | agcaactgccggaaaaatacaaggaaattttcttcgaccaaagcaagaac |
| | | | ggttatgcaggctatatcgacggtggtgcgtcgcaagaagaattttataa |
| | | | attcatcaaaccgattttggagaagatggatggtaccgaggagctgctgg |
| | | | taaaattgaatcgggaagacctcttgcgaaagcagcgtactttcgataat |
| | | | ggtagcatcccgcaccagattcatctgggcgaacttcacgcaatcctacg |
| | | | tcgtcaagaggatttctacccgtttctgaaggataaccgtgaaaagatcg |
| | | | agaagattctgacctttcgtattccgtattatgtgggtccgctggcgaga |
| | | | ggcaacagccgttttgcgtggatgacccgcaagtcggaagagactatcac |
| | | | gccgtggaatttcgaggaggtcgtggacaagggtgcatcagcgcaatctt |
| | | | ttattgaacgtatgaccaattttgataaaaaccttccaaatgaaaaagtg |
| | | | ttgccgaagcacagcttgctgtacgagtatttcaccgtttacaatgaact |
| | | | gacgaaagtcaagtacgttaccgaaggtatgcgtaagccagcctttctgt |
| | | | ctggggagcaaaaaaaagccattgtggatctgctgttcaagaccaatcgc |
| | | | aaggttaccgtcaagcaactgaaagaggattacttcaaaaaaatcgaatg |
| | | | ttttgactccgtggaaatcagcggcgttgaagaccgctttaatgcatctc |
| | | | tgggcacctaccacgatctactcaagatcatcaaggataaggacttcctg |
| | | | gacaacgaagaaaatgaggatatcttggaagatattgtgctgactttaac |
| | | | gttgttcgaggatcgtgagatgattgaagaacgcctcaaaacgtacgcac |
| | | | atctgtttgacgataaggtgatgaaacagctaaagcgccgtcgttacacc |
| | | | ggttggggtcgtctgtctagaaagctgattaacggcattcgtgacaaaca |
| | | | atctggcaagaccatcctggacttcctcaaatccgacggcttcgcaaatc |
| | | | gtaatttcatgcaactgatccatgatgatagcctgacctttaaagaagat |
| | | | attcagaaggctcaagtgtccggccagggtgactcgctgcatgagcatat |
| | | | cgcgaatctggctggctcccctgcgataaaaaaaggtattctccaaacgg |
| | | | tgaaagttgtggatgaactggttaaagtcatgggtcgtcataaaccggaa |
| | | | aacatcgtgattgagatggcacgtgagaaccagaccacccagaaaggtca |
| | | | aaaaaacagtcgtgagcgtatgaaacgtatcgaagaggggattaaggagt |
| | | | taggcagccagattctgaaggagcacccggttgagaacacccagctgcaa |
| | | | aacgagaagctctacctgtattacctgcaaaacggccgtgacatgtatgt |
| | | | tgaccaggagctggacatcaatcgtctcagcgactacgacgtggatgcta |
| | | | tcgtgccgcagagctttctaaaagatgattccatcgacaataaagtgctc |
| | | | acccgtagcgacaagaaccgtggtaaaagcgacaacgtgccgagcgagga |
| | | | agtagttaagaaaatgaaaaactactggcgtcagctattgaacgccaagc |
| | | | ttatcacccagcgtaagtttgacaacttgacgaaggcagagcgtggcggc |
| | | | ctgagcgagctagacaaggcgggttttattaaacgccagctggtggaaac |
| | | | tcgccagattaccaaacacgttgcgcaaatcttggactcacgcatgaata |
| | | | cgaagtacgacgaaaacgataaattgatccgcgaggtgaaggtcataacc |
| | | | ctgaaatctaaactggttagcgatttccggaaggactttcaattttataa |
| | | | agtccgggagattaacaactatcaccatgcacacgacgcttacttaaatg |
| | | | cggttgttggcacggccctgatcaagaagtacccgaaactggaaagcgag |
| | | | tttgtttatggtgactacaaggtgtatgacgttcgcaagatgattgcaaa |
| | | | atccgaacaagagattggtaaggcgacggctaagtattttttctacagca |
| | | | atatcatgaacttcttcaagactgaaatcaccctggcgaacggtgaaatt |
| | | | cgtaaacgtccgttgatcgaaaccaacggtgaaaccggtgaaatcgtttg |
| | | | ggataaaggtagggattttgcgaccgttcgtaaggtcctgagcatgccgc |
| | | | aggttaacatcgtgaaaaagaccgaagtgcagaccggcggtttctccaag |
| | | | gagagtatcttaccgaagcgcaacagtgacaagctgatcgcgcgtaaaaa |
| | | | agactgggatccgaaaaaatacggcggctttgactccccgaccgtcgcgt |
| | | | acagcgtactggtcgtggctaaagtggagaaaggcaaatcgaaaaagctg |
| | | | aaatccgttaaagagttgctgggtattacgattatggaacgttcgagctt |
| | | | cgagaaaaatccgatcgacttcctggaggccaaaggctacaaggaagtta |
| | | | aaaaagacctgatcattaaactgccgaagtacagcctgttcgagctcgag |
| | | | aacggtcgcaaacgcatgctggcgagcgcgggtgagctccaaaaaggcaa |
| | | | cgaattggctctgccgagcaagtacgtgaacttcctgtatctggcctccc |
| | | | attatgaaaaactgaagggtagcccggaagataacgaacaaaagcagctg |
| | | | ttcgtggaacaacataaacactatctggacgagattattgagcaaatcag |
| | | | cgagttcagcaaacgcgtgattctggcggatgctaatctggacaaggttc |
| | | | tttcagcctataataaacaccgtgataagccgatccgtgaacaggcagaa |
| | | | aatattattcacctgttcaccctgacgaacctgggtgcgccagcagcgtt |
| | | | taaatacttcgacacaacgatcgaccggaagcgttataccagcaccaaag |
| | | | aggttcttgacgccaccttgattcaccaaagcatcacgggtctgtatgaa |
| | | | acccgtattgacttgagccagttgggcggagattcagggggaagtggcgg |
| | | | aagcggtgggtctacgaacctgagcgacattattgaaaaagaaactggta |
| | | | aacagttggtgatccaagaatccattctgatgttaccggaggaagtggaa |
| | | | gaggtgatcggcaacaagccggagtccgacatcctggttcacaccgcgta |
| | | | tgatgagtcgaccgatgagaatgttatgctgctgaccagcgacgccccag |
| | | | agtacaaaccgtgggcgctcgtcatccaggatagcaatggcgaaaacaag |
| | | | atcaagatgttgtctggcggtagcatggctccgaaaaagaagcgtaaagt |
| | | | aggtattcatggtgttccggcatcaggttaa |
| 14 | Base editor | AmA1-dCasPhi-2 | atgggaagtcatcaccaccaccaccattcaggtgagaacctgtacttcca |
| | | | gagcatggctccgaaaaagaaacgtaaggtgggtatccacggcgttccgg |
| | | | cgatggctgattcaagtgaaaaaatgaggggacaatatatcagccgtgac |
| | | | acctttgagaaaaactacaaaccgattgacggcacgaaagaggcgcatct |
| | | | gctgtgcgaaattaaatggggtaagtacggcaagccgtggctgcattggt |
| | | | gtcaaaaccaacgtatgaatatccatgctgaagactacttcatgaacaac |
| | | | atcttcaaagccaaaaagcatccggtgcactgctacgtgacctggtattt |
| | | | gtcctggtcaccgtgcgcggattgtgcgagcaagatcgttaagttcctgg |
| | | | aggagcgtccttatctgaaactgaccatttatgttgcacagctgtattat |
| | | | cacactgaagaggagaaccgtaaaggtctgcgtttgctgcgttcgaagaa |
| | | | ggtgattatccgcgtgatggatatctctgattacaactactgctggaaag |
| | | | tctttgttagcaatcagaacggtaatgaagactactggccgctgcaattc |
| | | | gatccgtgggttaaggagaactacagccgcctcttggacattttctggga |
| | | | aagcaagtgccgttccccaaatccgtggtctggaagtgagacacccggga |
| | | | cttcagaatccgcgaccccggagtctggaatgccaaaacccgctgtagag |
| | | | tcagaattttcaaaagttttgaaaaagcactttccgggagaacgtttccg |
| | | | ctcgagctatatgaaaagaggcggtaagatcctggctgcccaaggcgaag |
| | | | aggcagttgttgcgtatctacagggcaagtccgaggaggagcctccgaac |
| | | | ttccagccaccggcgaagtgccacgtggtgaccaaatcccgtgacttcgc |
| | | | ggaatggccgatcatgaaggcgtcggaagcgattcaacgttatatctatg |
| | | | cactgtcgaccaccgaacgcgcggcctgtaaaccgggcaaaagtagtgag |
| | | | agccatgcagcgtggttcgccgctaccggtgtcagcaaccacggctacag |
| | | | ccacgttcagggtctgaacctgatttttgatcataccttgggccgttacg |
| | | | atggtgttctgaaaaaggtccaactgcgcaacgaaaaggcgagagcgcgt |
| | | | ttagagagcattaacgcgtctagagcagatgaaggtttaccggagattaa |
| | | | agccgaggaggaagaagtggctacgaacgaaaccggccacctgttgcaac |
| | | | cgccgggtatcaatccgagcttttacgtgtatcagaccattagcccgcag |
| | | | gcataccgtccgcgtgacgagatcgtgctgccgccggagtacgcaggtta |
| | | | cgtacgtgacccgaatgctccgattccgctgggtgtggtgcgtaaccgtt |
| | | | gcgatatccaaaaagggtgcccgggctacatcccggaatggcagcgtgaa |
| | | | gcaggtaccgcgatcagcccgaaaacaggcaaggccgtaactgtgccggg |
| | | | cctgtctccgaagaaaaacaaacgtatgcgtaggtactggcgctctgaga |
| | | | aggagaaagctcaagatgcgctgttggttaccgtacgcattggtaccgat |
| | | | tgggttgtcatcgacgtgcgcggtctgttgcgcaacgcccgttggcgcac |
| | | | catcgctcccaaggacatcagcctgaatgcgctcctcgatttgtttaccg |
| | | | gtgatccggtgatcgatgtgcgtcgcaatattgttacctttacgtacacc |
| | | | ctcgacgcctgtggtacctacgcccgtaaatggaccttgaaaggcaaaca |
| | | | gacgaaagcgaccctggacaaactgacggccactcagacggttgcgttgg |
| | | | ttgcgattgcgcttggtcagaccaacccgatttccgctggcattagccgt |
| | | | gttacacaagaaaatggtgcactgcaatgcgaaccgctggaccggttcac |
| | | | cctgccggatgacctgttgaaggacatcagcgcatatcgcattgcttggg |
| | | | acagaaacgaagaggaactacgcgctcgcagcgtcgaggccctgccggag |
| | | | gcgcaacaagcggaggtccgtgcgctggacggggtgtccaaagaaaccgc |
| | | | gcgtacccaactgtgcgcagatttcggcctggacccgaaacgtctgccgt |
| | | | gggataaaatgagctccaatactacctttatcagcgaagccttgctgtct |
| | | | aacagcgtgagccgtgatcaggtttttttcactccggctccgaagaaggg |
| | | | tgctaagaaaaaagcgcctgttgaagttatgcgtaaagaccgcacttggg |
| | | | cacgtgcgtacaagccgcgtctgagcgtcgaagctcagaaactgaagaac |
| | | | gaagccctgtgggcacttaagcgcacctccccggagtatttgaagctgag |
| | | | ccgtcgcaaagaggagctgtgtcgtcgtagcatcaactatgttatcgaaa |
| | | | agacccgtcgccgtacgcaatgtcagattgtgatcccggtgatcgaggat |
| | | | ctgaatgttcgcttctttcatggttctggtaagagactgcccggttggga |
| | | | taatttcttcaccgctaaaaaagagaaccgttggttcatccagggcctgc |
| | | | acaaagcgtttagcgatctgcgtacgcaccgttccttctatgtttttgaa |
| | | | gttcgtccggagcgcacctctattacctgtccaaagtgcggtcattgcga |
| | | | ggtgggtaatcgtgacggtgaggcgtttcagtgcctgagctgcggcaaga |
| | | | cgtgcaacgcagatcttgacgtcgcgacccataatctgacccaagtggcg |
| | | | ttgaccggtaagaccatgccgaagcgcgaggagccgcgtgacgctcaggg |
| | | | tacagcgccagcgcgcaagactaagaaggcgagcaaatcaaaggcgccac |
| | | | cggcggagcgcgaagaccagaccccggcgcaagagccaagccagaccagt |
| | | | tctggaggtagcgggggtagcggtggtagcactaacttaagtgatattat |
| | | | cgagaaagaaaccgggaaacagctggtgatccaagaaagcattctgatgc |
| | | | tgcccgaagaagttgaagaagtgattggtaacaagcctgagtcggacatc |
| | | | ttagtgcacaccgcgtacgatgaaagcacggatgagaatgtgatgctgct |
| | | | cacctctgatgcccccgaatacaagccgtgggctctggtgatccaggact |
| | | | ctaacggagaaaacaaaattaagatgctctctggcggctccatggcgcct |
| | | | aaaaaaaaacgcaaagtggggattcacggggttccggcgtaa |
| 15 | Base editor | AmA1-dLbCas12a | atgggaagtcatcaccaccaccaccattcaggtgagaacctgtacttcca |
| | | | gagcatggctccgaaaaagaaacgtaaggtgggtatccacggcgttccgg |
| | | | cgatggctgattcaagtgaaaaaatgaggggacaatatatcagccgtgac |
| | | | acctttgagaaaaactacaaaccgattgacggcacgaaagaggcgcatct |
| | | | gctgtgcgaaattaaatggggtaagtacggcaagccgtggctgcattggt |
| | | | gtcaaaaccaacgtatgaatatccatgctgaagactacttcatgaacaac |
| | | | atcttcaaagccaaaaagcatccggtgcactgctacgtgacctggtattt |
| | | | gtcctggtcaccgtgcgcggattgtgcgagcaagatcgttaagttcctgg |
| | | | aggagcgtccttatctgaaactgaccatttatgttgcacagctgtattat |
| | | | cacactgaagaggagaaccgtaaaggtctgcgtttgctgcgttcgaagaa |
| | | | ggtgattatccgcgtgatggatatctctgattacaactactgctggaaag |
| | | | tctttgttagcaatcagaacggtaatgaagactactggccgctgcaattc |
| | | | gatccgtgggttaaggagaactacagccgcctcttggacattttctggga |
| | | | aagcaagtgccgttccccaaatccgtggtctggaagtgagacacccggga |
| | | | cttcagaatccgcgaccccggagtctggaatgtcaaaactagaaaagttt |
| | | | acaaattgttactccctgtcgaaaaccctgagattcaaagcgatcccggt |
| | | | gggtaaaactcaagagaacattgataacaaacgtctgctggttgaggacg |
| | | | aaaagcgcgcggaggattacaaaggcgttaaaaagctgttggaccgttat |
| | | | tacctgtcctttattaatgatgttctgcacagcattaaattaaagaacct |
| | | | aaacaattacatctccctcttccgtaaaaagacccgcaccgaaaaggaaa |
| | | | ataaagaactggagaaccttgagattaatctgcgtaaagaaatcgctaaa |
| | | | gcgtttaaaggcaacgagggttataaatccttatttaaaaaagacatcat |
| | | | cgaaacgattctcccagaattcctggacgacaaagacgagatcgcgctgg |
| | | | ttaattccttcaacggctttaccaccgcattcaccggcttttttgataac |
| | | | cgcgaaaacatgttctctgaagaggccaaaagcacctcaattgcgtttcg |
| | | | ctgcattaacgaaaatctgacgcgttatatctctaacatggatatcttcg |
| | | | agaaggttgacgctatctttgataaacatgaagtccaggagatcaaggag |
| | | | aagatcctgaacagcgactacgacgtggaggatttcttcgagggtgagtt |
| | | | cttcaattttgttttgacccaagaaggcatcgatgtctacaatgcgataa |
| | | | ttggtggttttgttaccgagagcggggagaaaattaagggtctgaacgaa |
| | | | tatatcaacttgtacaaccagaaaaccaaacaaaaactgccgaagtttaa |
| | | | gccgctgtacaagcaagtcttgtcggatcgtgaatcgctgagcttttacg |
| | | | gcgaaggctacaccagcgatgaggaggtgttggaggtgttccgcaatacg |
| | | | ttgaataagaactctgaaattttttccagcattaagaagctggaaaaact |
| | | | attcaagaacttcgacgagtatagcagcgcaggtatctttgtgaaaaacg |
| | | | gtccggctattagcaccatctcgaaagacattttcggcgaatggaatgtt |
| | | | attcgtgataagtggaatgccgaatacgacgacattcatctgaagaaaaa |
| | | | ggcggttgtaactgaaaagtacgaggacgatcgtcgtaagtcgttcaaaa |
| | | | agatcggctccttctctctggagcaactgcaagagtacgccgatgccgat |
| | | | ttgtcagtcgtggagaagctgaaagagatcatcattcaaaaagttgacga |
| | | | aatctataaggtctatggtagcagtgaaaaactgtttgacgcggacttcg |
| | | | tgctggagaaaagcctgaagaaaaatgacgccgtggtggcaatcatgaaa |
| | | | gatcttctggatagcgtcaagtctttcgaaaactacatcaaagcgttttt |
| | | | cggtgaaggcaaggaaacgaatcgtgacgagagcttctatggtgatttcg |
| | | | tcctagcctatgatatcctgcttaaggtggatcacatctatgatgccatc |
| | | | cgcaactatgttacccagaaaccgtactcaaaggacaagttcaagttata |
| | | | cttccagaatccgcagtttatgggtggctgggataaagataaggaaaccg |
| | | | actatcgcgccaccattttacgttacggtagtaagtactatctggcaatc |
| | | | atggataaaaaatatgcgaaatgcctgcaaaagatcgacaaggacgatgt |
| | | | taacggcaattatgagaagatcaactacaaactgctgccgggcccaaata |
| | | | agatgctgccgaaggtgtttttcagcaaaaaatggatggcgtattacaat |
| | | | ccgagcgaggacatccaaaagatctacaaaaacggtacgtttaagaaggg |
| | | | tgatatgttcaacctcaacgattgtcataaacttattgacttcttcaagg |
| | | | atagcatatctcgttatccgaagtggagcaacgcgtatgactttaacttt |
| | | | agcgaaaccgagaaatacaaagatatcgcgggtttctatcgtgaagttga |
| | | | agaacagggttacaaagtgtccttcgagtccgcgagcaagaaggaggtag |
| | | | acaagctggtcgaggagggtaagttgtacatgtttcagatttacaataag |
| | | | gacttttccgacaagtcccatggtacaccgaatttgcacaccatgtattt |
| | | | caagcttctgttcgacgagaacaaccacggccagatccgtctgagcggtg |
| | | | gtgcagagctgttcatgcgtagagctagcctgaagaaagaagagttggtc |
| | | | gttcatccggctaattcgccgatcgcgaacaaaaatccagataaccctaa |
| | | | aaagactacgaccctgagctatgatgtgtataaagacaaacgctttagcg |
| | | | aagaccagtatgagttacacattccgatcgctatcaacaagtgcccgaag |
| | | | aatattttcaagatcaacaccgaagtgcgtgttcttttgaagcacgatga |
| | | | caacccgtatgtgattggtattgctcgtggtgaacgtaatctgctgtaca |
| | | | ttgtggttgtggacggcaaaggaaacatcgtggaacagtacagcctgaac |
| | | | gaaatcattaacaatttcaacggcattcgcattaagacggattaccattc |
| | | | tttgctggacaaaaaggagaaagagcgtttcgaggcccgtcagaactgga |
| | | | cctccatcgagaacattaaggagctgaaggcgggttacatcagccaggtg |
| | | | gtgcacaaaatctgcgaactggtggagaagtacgacgcggtgattgcttt |
| | | | ggcggatttgaactccggttttaagaacagccgcgttaaagtggaaaagc |
| | | | aggtttatcagaaattcgaaaaaatgctgattgataaattgaattacatg |
| | | | gttgacaaaaagtctaacccgtgtgcaaccggcggtgcgctgaagggcta |
| | | | ccaaatcaccaataagtttgaaagctttaagagcatgagcacgcaaaatg |
| | | | gcttcattttttacattccggcgtggctgaccagcaagatcgatccgtca |
| | | | accggctttgttaatttactgaagacgaagtatacctctattgccgactc |
| | | | taaaaaatttattagttccttcgatagaatcatgtatgttccggaagagg |
| | | | acctgttcgaatttgcactggactacaagaactttagccgtaccgatgct |
| | | | gattatataaagaaatggaaactctacagctatggcaaccgcatccgtat |
| | | | ctttcgcaacccgaaaaaaaacaacgtgttcgactgggaagaggtatgtc |
| | | | tgacttcagcgtacaaggagcttttcaataaatatggtatcaactaccag |
| | | | cagggtgatatccgtgcactgctttgcgagcaaagcgataaagcgttcta |
| | | | ttcaagcttcatggcgctgatgagcctgatgctgcaaatgcgcaacagca |
| | | | tcaccggtcgtaccgatgttgcatttctgatttctccagtaaaaaacagc |
| | | | gatggtatcttttacgacagccgtaattacgaggcccaagaaaatgcgat |
| | | | tttgccgaagaacgctgacgcgaatggtgcatacaacattgcaaggaaag |
| | | | ttctgtgggcaatcggccaatttaaaaaagcggaagatgagaagctggac |
| | | | aaagtgaaaattgcgattagcaacaaggagtggctggagtatgcgcagac |
| | | | ctccgttaaatcagggggaagtggcggaagcggtgggtctacgaacctga |
| | | | gcgacattattgaaaaagaaactggtaaacagttggtgatccaagaatcc |
| | | | attctgatgttaccggaggaagtggaagaggtgatcggcaacaagccgga |
| | | | gtccgacatcctggttcacaccgcgtatgatgagtcgaccgatgagaatg |
| | | | ttatgctgctgaccagcgacgccccagagtacaaaccgtgggcgctcgtc |
| | | | atccaggatagcaatggcgaaaacaagatcaagatgttgtctggcggtag |
| | | | catggctccgaaaaagaagcgtaaagtaggtattcatggtgttccggcat |
| | | | caggttaa |
| 16 | Base editor | Anc-rA1-dCas9 | atgggaagtcatcaccaccaccaccattcaggtgagaacctgtacttcca |
| | | | gagcatggctccgaaaaagaaacgtaaggtgggtatccacggcgttccgg |
| | | | cgatgagttcagaaacaggacccgttgctgtagacccgacgctgcgccgt |
| | | | cgtattgagccgcacgagttcgaggttttttttgatccgcgtgaactccg |
| | | | caaggagacatgcttgctgtacgagatcaaatggggcacttcacacaaaa |
| | | | tctggcgccacagcagcaagaacaccaccaagcacgtggaagtgaacttc |
| | | | atcgagaaattcacgtctgaacgtcacttttgtccgtccaccagctgtag |
| | | | cattacctggtttctatcctggagcccgtgcggtgaatgttccaaggcga |
| | | | ttaccgaattcttgtcgcaacatccgaacgtgactcttgttatttatgtg |
| | | | gcccgtttgtaccatcacatggatcagcagaatcgtcaaggcttgcgcga |
| | | | cctggttaacagcggtgttaccatccagatcatgaccgcaccggaatacg |
| | | | actactgctggcgtaatttcgtcaactatccgccgggcaaggaggcgcat |
| | | | tggccgcgttatccgccgctgtggatgaaactgtacgctctggaactgca |
| | | | tgcgggtattttaggcctgccaccgtgcttgaatattctgagacgcaagc |
| | | | agcctcagctgacctttttcacgatcgcgctgcaaagctgtcattatcaa |
| | | | cgtctgccgccacacatcctgtgggctaccggtctgaaatctggaagtga |
| | | | gacacccgggacttcagaatccgcgaccccggagtctggaatggataaaa |
| | | | agtattcaataggactagctattggcaccaacagcgttggttgggcagtt |
| | | | atcaccgatgaatataaggtcccgagcaagaagtttaaggtcctgggcaa |
| | | | caccgatcgccacagcataaaaaagaaccttattggtgcgctgctgttcg |
| | | | attctggtgagactgccgaagcgacccgtctgaagagaaccgcgcgtcgt |
| | | | agatacacccggcgcaagaaccgtatctgctatctgcaagaaattttcag |
| | | | caacgagatggctaaggtcgacgactccttctttcatcgcctggaggaaa |
| | | | gctttctggttgaggaagacaaaaagcacgagcgtcatccgattttcggt |
| | | | aacattgttgatgaagttgcctaccacgaaaaataccccacaatttatca |
| | | | tctgcgtaaaaaactggtagactccaccgataaggcggatttacgcctta |
| | | | tctatttggcgctggcgcatatgattaagttccgcggtcattttttgatc |
| | | | gagggcgacctgaatccggataacagcgacgttgacaaactgtttatcca |
| | | | gctggtgcagacctataaccagttgttcgaagagaacccgattaatgcgt |
| | | | ctggcgtggacgcgaaggcgatcctgagcgctcgtctgtccaagtcacgc |
| | | | cgcctggagaacttgatcgcgcaactgccgggtgagaagaaaaacggctt |
| | | | gttcggcaatctgattgctctgtccctaggtttgaccccgaatttcaaat |
| | | | ctaattttgatctggcggaagatgcgaaattacagttgagcaaggacact |
| | | | tacgatgacgatctggacaacctgctcgctcagatcggcgatcagtatgc |
| | | | cgatcttttcctggccgcgaagaacctgagcgatgccatcttgctatcgg |
| | | | acatcttacgtgttaacaccgaaatcaccaaagctccgctaagcgcgtcc |
| | | | atgattaaacgttacgatgaacaccaccaggatctgacgctgctgaaagc |
| | | | gttggtgcgccagcaactgccggaaaaatacaaggaaattttcttcgacc |
| | | | aaagcaagaacggttatgcaggctatatcgacggtggtgcgtcgcaagaa |
| | | | gaattttataaattcatcaaaccgattttggagaagatggatggtaccga |
| | | | ggagctgctggtaaaattgaatcgggaagacctcttgcgaaagcagcgta |
| | | | ctttcgataatggtagcatcccgcaccagattcatctgggcgaacttcac |
| | | | gcaatcctacgtcgtcaagaggatttctacccgtttctgaaggataaccg |
| | | | tgaaaagatcgagaagattctgacctttcgtattccgtattatgtgggtc |
| | | | cgctggcgagaggcaacagccgttttgcgtggatgacccgcaagtcggaa |
| | | | gagactatcacgccgtggaatttcgaggaggtcgtggacaagggtgcatc |
| | | | agcgcaatcttttattgaacgtatgaccaattttgataaaaaccttccaa |
| | | | atgaaaaagtgttgccgaagcacagcttgctgtacgagtatttcaccgtt |
| | | | tacaatgaactgacgaaagtcaagtacgttaccgaaggtatgcgtaagcc |
| | | | agcctttctgtctggggagcaaaaaaaagccattgtggatctgctgttca |
| | | | agaccaatcgcaaggttaccgtcaagcaactgaaagaggattacttcaaa |
| | | | aaaatcgaatgttttgactccgtggaaatcagcggcgttgaagaccgctt |
| | | | taatgcatctctgggcacctaccacgatctactcaagatcatcaaggata |
| | | | aggacttcctggacaacgaagaaaatgaggatatcttggaagatattgtg |
| | | | ctgactttaacgttgttcgaggatcgtgagatgattgaagaacgcctcaa |
| | | | aacgtacgcacatctgtttgacgataaggtgatgaaacagctaaagcgcc |
| | | | gtcgttacaccggttggggtcgtctgtctagaaagctgattaacggcatt |
| | | | cgtgacaaacaatctggcaagaccatcctggacttcctcaaatccgacgg |
| | | | cttcgcaaatcgtaatttcatgcaactgatccatgatgatagcctgacct |
| | | | ttaaagaagatattcagaaggctcaagtgtccggccagggtgactcgctg |
| | | | catgagcatatcgcgaatctggctggctcccctgcgataaaaaaaggtat |
| | | | tctccaaacggtgaaagttgtggatgaactggttaaagtcatgggtcgtc |
| | | | ataaaccggaaaacatcgtgattgagatggcacgtgagaaccagaccacc |
| | | | cagaaaggtcaaaaaaacagtcgtgagcgtatgaaacgtatcgaagaggg |
| | | | gattaaggagttaggcagccagattctgaaggagcacccggttgagaaca |
| | | | cccagctgcaaaacgagaagctctacctgtattacctgcaaaacggccgt |
| | | | gacatgtatgttgaccaggagctggacatcaatcgtctcagcgactacga |
| | | | cgtggatgctatcgtgccgcagagctttctaaaagatgattccatcgaca |
| | | | ataaagtgctcacccgtagcgacaagaaccgtggtaaaagcgacaacgtg |
| | | | ccgagcgaggaagtagttaagaaaatgaaaaactactggcgtcagctatt |
| | | | gaacgccaagcttatcacccagcgtaagtttgacaacttgacgaaggcag |
| | | | agcgtggcggcctgagcgagctagacaaggcgggttttattaaacgccag |
| | | | ctggtggaaactcgccagattaccaaacacgttgcgcaaatcttggactc |
| | | | acgcatgaatacgaagtacgacgaaaacgataaattgatccgcgaggtga |
| | | | aggtcataaccctgaaatctaaactggttagcgatttccggaaggacttt |
| | | | caattttataaagtccgggagattaacaactatcaccatgcacacgacgc |
| | | | ttacttaaatgcggttgttggcacggccctgatcaagaagtacccgaaac |
| | | | tggaaagcgagtttgtttatggtgactacaaggtgtatgacgttcgcaag |
| | | | atgattgcaaaatccgaacaagagattggtaaggcgacggctaagtattt |
| | | | tttctacagcaatatcatgaacttcttcaagactgaaatcaccctggcga |
| | | | acggtgaaattcgtaaacgtccgttgatcgaaaccaacggtgaaaccggt |
| | | | gaaatcgtttgggataaaggtagggattttgcgaccgttcgtaaggtcct |
| | | | gagcatgccgcaggttaacatcgtgaaaaagaccgaagtgcagaccggcg |
| | | | gtttctccaaggagagtatcttaccgaagcgcaacagtgacaagctgatc |
| | | | gcgcgtaaaaaagactgggatccgaaaaaatacggcggctttgactcccc |
| | | | gaccgtcgcgtacagcgtactggtcgtggctaaagtggagaaaggcaaat |
| | | | cgaaaaagctgaaatccgttaaagagttgctgggtattacgattatggaa |
| | | | cgttcgagcttcgagaaaaatccgatcgacttcctggaggccaaaggcta |
| | | | caaggaagttaaaaaagacctgatcattaaactgccgaagtacagcctgt |
| | | | tcgagctcgagaacggtcgcaaacgcatgctggcgagcgcgggtgagctc |
| | | | caaaaaggcaacgaattggctctgccgagcaagtacgtgaacttcctgta |
| | | | tctggcctcccattatgaaaaactgaagggtagcccggaagataacgaac |
| | | | aaaagcagctgttcgtggaacaacataaacactatctggacgagattatt |
| | | | gagcaaatcagcgagttcagcaaacgcgtgattctggcggatgctaatct |
| | | | ggacaaggttctttcagcctataataaacaccgtgataagccgatccgtg |
| | | | aacaggcagaaaatattattcacctgttcaccctgacgaacctgggtgcg |
| | | | ccagcagcgtttaaatacttcgacacaacgatcgaccggaagcgttatac |
| | | | cagcaccaaagaggttcttgacgccaccttgattcaccaaagcatcacgg |
| | | | gtctgtatgaaacccgtattgacttgagccagttgggcggagattcaggg |
| | | | ggaagtggcggaagcggtgggtctacgaacctgagcgacattattgaaaa |
| | | | agaaactggtaaacagttggtgatccaagaatccattctgatgttaccgg |
| | | | aggaagtggaagaggtgatcggcaacaagccggagtccgacatcctggtt |
| | | | cacaccgcgtatgatgagtcgaccgatgagaatgttatgctgctgaccag |
| | | | cgacgccccagagtacaaaccgtgggcgctcgtcatccaggatagcaatg |
| | | | gcgaaaacaagatcaagatgttgtctggcggtagcatggctccgaaaaag |
| | | | aagcgtaaagtaggtattcatggtgttccggcatcaggttaa |
| 17 | Base editor | Anc-rA1- | atgggaagtcatcaccaccaccaccattcaggtgagaacctgtacttcca |
| | | dCasPhi-2 | gagcatggctccgaaaaagaaacgtaaggtgggtatccacggcgttccgg |
| | | | cgatgagttcagaaacaggacccgttgctgtagacccgacgctgcgccgt |
| | | | cgtattgagccgcacgagttcgaggttttttttgatccgcgtgaactccg |
| | | | caaggagacatgcttgctgtacgagatcaaatggggcacttcacacaaaa |
| | | | tctggcgccacagcagcaagaacaccaccaagcacgtggaagtgaacttc |
| | | | atcgagaaattcacgtctgaacgtcacttttgtccgtccaccagctgtag |
| | | | cattacctggtttctatcctggagcccgtgcggtgaatgttccaaggcga |
| | | | ttaccgaattcttgtcgcaacatccgaacgtgactcttgttatttatgtg |
| | | | gcccgtttgtaccatcacatggatcagcagaatcgtcaaggcttgcgcga |
| | | | cctggttaacagcggtgttaccatccagatcatgaccgcaccggaatacg |
| | | | actactgctggcgtaatttcgtcaactatccgccgggcaaggaggcgcat |
| | | | tggccgcgttatccgccgctgtggatgaaactgtacgctctggaactgca |
| | | | tgcgggtattttaggcctgccaccgtgcttgaatattctgagacgcaagc |
| | | | agcctcagctgacctttttcacgatcgcgctgcaaagctgtcattatcaa |
| | | | cgtctgccgccacacatcctgtgggctaccggtctgaaatctggaagtga |
| | | | gacacccgggacttcagaatccgcgaccccggagtctggaatgccaaaac |
| | | | ccgctgtagagtcagaattttcaaaagttttgaaaaagcactttccggga |
| | | | gaacgtttccgctcgagctatatgaaaagaggcggtaagatcctggctgc |
| | | | ccaaggcgaagaggcagttgttgcgtatctacagggcaagtccgaggagg |
| | | | agcctccgaacttccagccaccggcgaagtgccacgtggtgaccaaatcc |
| | | | cgtgacttcgcggaatggccgatcatgaaggcgtcggaagcgattcaacg |
| | | | ttatatctatgcactgtcgaccaccgaacgcgcggcctgtaaaccgggca |
| | | | aaagtagtgagagccatgcagcgtggttcgccgctaccggtgtcagcaac |
| | | | cacggctacagccacgttcagggtctgaacctgatttttgatcatacctt |
| | | | gggccgttacgatggtgttctgaaaaaggtccaactgcgcaacgaaaagg |
| | | | cgagagcgcgtttagagagcattaacgcgtctagagcagatgaaggttta |
| | | | ccggagattaaagccgaggaggaagaagtggctacgaacgaaaccggcca |
| | | | cctgttgcaaccgccgggtatcaatccgagcttttacgtgtatcagacca |
| | | | ttagcccgcaggcataccgtccgcgtgacgagatcgtgctgccgccggag |
| | | | tacgcaggttacgtacgtgacccgaatgctccgattccgctgggtgtggt |
| | | | gcgtaaccgttgcgatatccaaaaagggtgcccgggctacatcccggaat |
| | | | ggcagcgtgaagcaggtaccgcgatcagcccgaaaacaggcaaggccgta |
| | | | actgtgccgggcctgtctccgaagaaaaacaaacgtatgcgtaggtactg |
| | | | gcgctctgagaaggagaaagctcaagatgcgctgttggttaccgtacgca |
| | | | ttggtaccgattgggttgtcatcgacgtgcgcggtctgttgcgcaacgcc |
| | | | cgttggcgcaccatcgctcccaaggacatcagcctgaatgcgctcctcga |
| | | | tttgtttaccggtgatccggtgatcgatgtgcgtcgcaatattgttacct |
| | | | ttacgtacaccctcgacgcctgtggtacctacgcccgtaaatggaccttg |
| | | | aaaggcaaacagacgaaagcgaccctggacaaactgacggccactcagac |
| | | | ggttgcgttggttgcgattgcgcttggtcagaccaacccgatttccgctg |
| | | | gcattagccgtgttacacaagaaaatggtgcactgcaatgcgaaccgctg |
| | | | gaccggttcaccctgccggatgacctgttgaaggacatcagcgcatatcg |
| | | | cattgcttgggacagaaacgaagaggaactacgcgctcgcagcgtcgagg |
| | | | ccctgccggaggcgcaacaagcggaggtccgtgcgctggacggggtgtcc |
| | | | aaagaaaccgcgcgtacccaactgtgcgcagatttcggcctggacccgaa |
| | | | acgtctgccgtgggataaaatgagctccaatactacctttatcagcgaag |
| | | | ccttgctgtctaacagcgtgagccgtgatcaggtttttttcactccggct |
| | | | ccgaagaagggtgctaagaaaaaagcgcctgttgaagttatgcgtaaaga |
| | | | ccgcacttgggcacgtgcgtacaagccgcgtctgagcgtcgaagctcaga |
| | | | aactgaagaacgaagccctgtgggcacttaagcgcacctccccggagtat |
| | | | ttgaagctgagccgtcgcaaagaggagctgtgtcgtcgtagcatcaacta |
| | | | tgttatcgaaaagacccgtcgccgtacgcaatgtcagattgtgatcccgg |
| | | | tgatcgaggatctgaatgttcgcttctttcatggttctggtaagagactg |
| | | | cccggttgggataatttcttcaccgctaaaaaagagaaccgttggttcat |
| | | | ccagggcctgcacaaagcgtttagcgatctgcgtacgcaccgttccttct |
| | | | atgtttttgaagttcgtccggagcgcacctctattacctgtccaaagtgc |
| | | | ggtcattgcgaggtgggtaatcgtgacggtgaggcgtttcagtgcctgag |
| | | | ctgcggcaagacgtgcaacgcagatcttgacgtcgcgacccataatctga |
| | | | cccaagtggcgttgaccggtaagaccatgccgaagcgcgaggagccgcgt |
| | | | gacgctcagggtacagcgccagcgcgcaagactaagaaggcgagcaaatc |
| | | | aaaggcgccaccggcggagcgcgaagaccagaccccggcgcaagagccaa |
| | | | gccagaccagttctggaggtagcgggggtagcggtggtagcactaactta |
| | | | agtgatattatcgagaaagaaaccgggaaacagctggtgatccaagaaag |
| | | | cattctgatgctgcccgaagaagttgaagaagtgattggtaacaagcctg |
| | | | agtcggacatcttagtgcacaccgcgtacgatgaaagcacggatgagaat |
| | | | gtgatgctgctcacctctgatgcccccgaatacaagccgtgggctctggt |
| | | | gatccaggactctaacggagaaaacaaaattaagatgctctctggcggct |
| | | | ccatggcgcctaaaaaaaaacgcaaagtggggattcacggggttccggcg |
| | | | taa |
| 18 | Base editor | Anc-rA1- | atgggaagtcatcaccaccaccaccattcaggtgagaacctgtacttcca |
| | | dLbCas12a | gagcatggctccgaaaaagaaacgtaaggtgggtatccacggcgttccgg |
| | | | cgatgagttcagaaacaggacccgttgctgtagacccgacgctgcgccgt |
| | | | cgtattgagccgcacgagttcgaggttttttttgatccgcgtgaactccg |
| | | | caaggagacatgcttgctgtacgagatcaaatggggcacttcacacaaaa |
| | | | tctggcgccacagcagcaagaacaccaccaagcacgtggaagtgaacttc |
| | | | atcgagaaattcacgtctgaacgtcacttttgtccgtccaccagctgtag |
| | | | cattacctggtttctatcctggagcccgtgcggtgaatgttccaaggcga |
| | | | ttaccgaattcttgtcgcaacatccgaacgtgactcttgttatttatgtg |
| | | | gcccgtttgtaccatcacatggatcagcagaatcgtcaaggcttgcgcga |
| | | | cctggttaacagcggtgttaccatccagatcatgaccgcaccggaatacg |
| | | | actactgctggcgtaatttcgtcaactatccgccgggcaaggaggcgcat |
| | | | tggccgcgttatccgccgctgtggatgaaactgtacgctctggaactgca |
| | | | tgcgggtattttaggcctgccaccgtgcttgaatattctgagacgcaagc |
| | | | agcctcagctgacctttttcacgatcgcgctgcaaagctgtcattatcaa |
| | | | cgtctgccgccacacatcctgtgggctaccggtctgaaatctggaagtga |
| | | | gacacccgggacttcagaatccgcgaccccggagtctggaatgtcaaaac |
| | | | tagaaaagtttacaaattgttactccctgtcgaaaaccctgagattcaaa |
| | | | gcgatcccggtgggtaaaactcaagagaacattgataacaaacgtctgct |
| | | | ggttgaggacgaaaagcgcgcggaggattacaaaggcgttaaaaagctgt |
| | | | tggaccgttattacctgtcctttattaatgatgttctgcacagcattaaa |
| | | | ttaaagaacctaaacaattacatctccctcttccgtaaaaagacccgcac |
| | | | cgaaaaggaaaataaagaactggagaaccttgagattaatctgcgtaaag |
| | | | aaatcgctaaagcgtttaaaggcaacgagggttataaatccttatttaaa |
| | | | aaagacatcatcgaaacgattctcccagaattcctggacgacaaagacga |
| | | | gatcgcgctggttaattccttcaacggctttaccaccgcattcaccggct |
| | | | tttttgataaccgcgaaaacatgttctctgaagaggccaaaagcacctca |
| | | | attgcgtttcgctgcattaacgaaaatctgacgcgttatatctctaacat |
| | | | ggatatcttcgagaaggttgacgctatctttgataaacatgaagtccagg |
| | | | agatcaaggagaagatcctgaacagcgactacgacgtggaggatttcttc |
| | | | gagggtgagttcttcaattttgttttgacccaagaaggcatcgatgtcta |
| | | | caatgcgataattggtggttttgttaccgagagcggggagaaaattaagg |
| | | | gtctgaacgaatatatcaacttgtacaaccagaaaaccaaacaaaaactg |
| | | | ccgaagtttaagccgctgtacaagcaagtcttgtcggatcgtgaatcgct |
| | | | gagcttttacggcgaaggctacaccagcgatgaggaggtgttggaggtgt |
| | | | tccgcaatacgttgaataagaactctgaaattttttccagcattaagaag |
| | | | ctggaaaaactattcaagaacttcgacgagtatagcagcgcaggtatctt |
| | | | tgtgaaaaacggtccggctattagcaccatctcgaaagacattttcggcg |
| | | | aatggaatgttattcgtgataagtggaatgccgaatacgacgacattcat |
| | | | ctgaagaaaaaggcggttgtaactgaaaagtacgaggacgatcgtcgtaa |
| | | | gtcgttcaaaaagatcggctccttctctctggagcaactgcaagagtacg |
| | | | ccgatgccgatttgtcagtcgtggagaagctgaaagagatcatcattcaa |
| | | | aaagttgacgaaatctataaggtctatggtagcagtgaaaaactgtttga |
| | | | cgcggacttcgtgctggagaaaagcctgaagaaaaatgacgccgtggtgg |
| | | | caatcatgaaagatcttctggatagcgtcaagtctttcgaaaactacatc |
| | | | aaagcgtttttcggtgaaggcaaggaaacgaatcgtgacgagagcttcta |
| | | | tggtgatttcgtcctagcctatgatatcctgcttaaggtggatcacatct |
| | | | atgatgccatccgcaactatgttacccagaaaccgtactcaaaggacaag |
| | | | ttcaagttatacttccagaatccgcagtttatgggtggctgggataaaga |
| | | | taaggaaaccgactatcgcgccaccattttacgttacggtagtaagtact |
| | | | atctggcaatcatggataaaaaatatgcgaaatgcctgcaaaagatcgac |
| | | | aaggacgatgttaacggcaattatgagaagatcaactacaaactgctgcc |
| | | | gggcccaaataagatgctgccgaaggtgtttttcagcaaaaaatggatgg |
| | | | cgtattacaatccgagcgaggacatccaaaagatctacaaaaacggtacg |
| | | | tttaagaagggtgatatgttcaacctcaacgattgtcataaacttattga |
| | | | cttcttcaaggatagcatatctcgttatccgaagtggagcaacgcgtatg |
| | | | actttaactttagcgaaaccgagaaatacaaagatatcgcgggtttctat |
| | | | cgtgaagttgaagaacagggttacaaagtgtccttcgagtccgcgagcaa |
| | | | gaaggaggtagacaagctggtcgaggagggtaagttgtacatgtttcaga |
| | | | tttacaataaggacttttccgacaagtcccatggtacaccgaatttgcac |
| | | | accatgtatttcaagcttctgttcgacgagaacaaccacggccagatccg |
| | | | tctgagcggtggtgcagagctgttcatgcgtagagctagcctgaagaaag |
| | | | aagagttggtcgttcatccggctaattcgccgatcgcgaacaaaaatcca |
| | | | gataaccctaaaaagactacgaccctgagctatgatgtgtataaagacaa |
| | | | acgctttagcgaagaccagtatgagttacacattccgatcgctatcaaca |
| | | | agtgcccgaagaatattttcaagatcaacaccgaagtgcgtgttcttttg |
| | | | aagcacgatgacaacccgtatgtgattggtattgctcgtggtgaacgtaa |
| | | | tctgctgtacattgtggttgtggacggcaaaggaaacatcgtggaacagt |
| | | | acagcctgaacgaaatcattaacaatttcaacggcattcgcattaagacg |
| | | | gattaccattctttgctggacaaaaaggagaaagagcgtttcgaggcccg |
| | | | tcagaactggacctccatcgagaacattaaggagctgaaggcgggttaca |
| | | | tcagccaggtggtgcacaaaatctgcgaactggtggagaagtacgacgcg |
| | | | gtgattgctttggcggatttgaactccggttttaagaacagccgcgttaa |
| | | | agtggaaaagcaggtttatcagaaattcgaaaaaatgctgattgataaat |
| | | | tgaattacatggttgacaaaaagtctaacccgtgtgcaaccggcggtgcg |
| | | | ctgaagggctaccaaatcaccaataagtttgaaagctttaagagcatgag |
| | | | cacgcaaaatggcttcattttttacattccggcgtggctgaccagcaaga |
| | | | tcgatccgtcaaccggctttgttaatttactgaagacgaagtatacctct |
| | | | attgccgactctaaaaaatttattagttccttcgatagaatcatgtatgt |
| | | | tccggaagaggacctgttcgaatttgcactggactacaagaactttagcc |
| | | | gtaccgatgctgattatataaagaaatggaaactctacagctatggcaac |
| | | | cgcatccgtatctttcgcaacccgaaaaaaaacaacgtgttcgactggga |
| | | | agaggtatgtctgacttcagcgtacaaggagcttttcaataaatatggta |
| | | | tcaactaccagcagggtgatatccgtgcactgctttgcgagcaaagcgat |
| | | | aaagcgttctattcaagcttcatggcgctgatgagcctgatgctgcaaat |
| | | | gcgcaacagcatcaccggtcgtaccgatgttgcatttctgatttctccag |
| | | | taaaaaacagcgatggtatcttttacgacagccgtaattacgaggcccaa |
| | | | gaaaatgcgattttgccgaagaacgctgacgcgaatggtgcatacaacat |
| | | | tgcaaggaaagttctgtgggcaatcggccaatttaaaaaagcggaagatg |
| | | | agaagctggacaaagtgaaaattgcgattagcaacaaggagtggctggag |
| | | | tatgcgcagacctccgttaaatcagggggaagtggcggaagcggtgggtc |
| | | | tacgaacctgagcgacattattgaaaaagaaactggtaaacagttggtga |
| | | | tccaagaatccattctgatgttaccggaggaagtggaagaggtgatcggc |
| | | | aacaagccggagtccgacatcctggttcacaccgcgtatgatgagtcgac |
| | | | cgatgagaatgttatgctgctgaccagcgacgccccagagtacaaaccgt |
| | | | gggcgctcgtcatccaggatagcaatggcgaaaacaagatcaagatgttg |
| | | | tctggcggtagcatggctccgaaaaagaagcgtaaagtaggtattcatgg |
| | | | tgttccggcatcaggttaa |
| 19 | Base editor | eA3A-dCas9 | atgggaagtcatcaccaccaccaccattcaggtgagaacctgtacttcca |
| | | | gagcatggctccgaaaaagaaacgtaaggtgggtatccacggcgttccgg |
| | | | cgatggaagcaagtcccgcttcaggaccaaggcacctgatggacccacat |
| | | | attttcaccagtaattttaacaacggcatcggccgtcacaagacctacct |
| | | | gtgttatgaggtcgaacgtctggataatggcacttccgtgaaaatggatc |
| | | | agcatcgtggtttcctgcatggtcaggctaagaatctgctttgcggtttc |
| | | | tatggtcgccacgccgaactgcgttttttggacctggttccgtccctcca |
| | | | actggacccggcgcaaatctatcgtgttacctggtttatcagctggtcgc |
| | | | cgtgtttcagctggggttgcgcaggcgaagtacgtgcgttcttacaagag |
| | | | aacacccatgtgcgcttgcgcatcttcgccgcacgtatttacgactacga |
| | | | cccgctgtataaagaggcgttgcaaatgctgcgcgatgctggcgcacagg |
| | | | ttagcattatgacgtacgacgaatttaagcactgctgggataccttcgtg |
| | | | gatcaccagggctgcccgtttcagccgtgggatggtttggacgagcactc |
| | | | tcaggcgctgagcggtcgtctcagagcgattctgcaaaaccagggaaact |
| | | | ctggaagtgagacacccgggacttcagaatccgcgaccccggagtctgga |
| | | | atggataaaaagtattcaataggactagctattggcaccaacagcgttgg |
| | | | ttgggcagttatcaccgatgaatataaggtcccgagcaagaagtttaagg |
| | | | tcctgggcaacaccgatcgccacagcataaaaaagaaccttattggtgcg |
| | | | ctgctgttcgattctggtgagactgccgaagcgacccgtctgaagagaac |
| | | | cgcgcgtcgtagatacacccggcgcaagaaccgtatctgctatctgcaag |
| | | | aaattttcagcaacgagatggctaaggtcgacgactccttctttcatcgc |
| | | | ctggaggaaagctttctggttgaggaagacaaaaagcacgagcgtcatcc |
| | | | gattttcggtaacattgttgatgaagttgcctaccacgaaaaatacccca |
| | | | caatttatcatctgcgtaaaaaactggtagactccaccgataaggcggat |
| | | | ttacgccttatctatttggcgctggcgcatatgattaagttccgcggtca |
| | | | ttttttgatcgagggcgacctgaatccggataacagcgacgttgacaaac |
| | | | tgtttatccagctggtgcagacctataaccagttgttcgaagagaacccg |
| | | | attaatgcgtctggcgtggacgcgaaggcgatcctgagcgctcgtctgtc |
| | | | caagtcacgccgcctggagaacttgatcgcgcaactgccgggtgagaaga |
| | | | aaaacggcttgttcggcaatctgattgctctgtccctaggtttgaccccg |
| | | | aatttcaaatctaattttgatctggcggaagatgcgaaattacagttgag |
| | | | caaggacacttacgatgacgatctggacaacctgctcgctcagatcggcg |
| | | | atcagtatgccgatcttttcctggccgcgaagaacctgagcgatgccatc |
| | | | ttgctatcggacatcttacgtgttaacaccgaaatcaccaaagctccgct |
| | | | aagcgcgtccatgattaaacgttacgatgaacaccaccaggatctgacgc |
| | | | tgctgaaagcgttggtgcgccagcaactgccggaaaaatacaaggaaatt |
| | | | ttcttcgaccaaagcaagaacggttatgcaggctatatcgacggtggtgc |
| | | | gtcgcaagaagaattttataaattcatcaaaccgattttggagaagatgg |
| | | | atggtaccgaggagctgctggtaaaattgaatcgggaagacctcttgcga |
| | | | aagcagcgtactttcgataatggtagcatcccgcaccagattcatctggg |
| | | | cgaacttcacgcaatcctacgtcgtcaagaggatttctacccgtttctga |
| | | | aggataaccgtgaaaagatcgagaagattctgacctttcgtattccgtat |
| | | | tatgtgggtccgctggcgagaggcaacagccgttttgcgtggatgacccg |
| | | | caagtcggaagagactatcacgccgtggaatttcgaggaggtcgtggaca |
| | | | agggtgcatcagcgcaatcttttattgaacgtatgaccaattttgataaa |
| | | | aaccttccaaatgaaaaagtgttgccgaagcacagcttgctgtacgagta |
| | | | tttcaccgtttacaatgaactgacgaaagtcaagtacgttaccgaaggta |
| | | | tgcgtaagccagcctttctgtctggggagcaaaaaaaagccattgtggat |
| | | | ctgctgttcaagaccaatcgcaaggttaccgtcaagcaactgaaagagga |
| | | | ttacttcaaaaaaatcgaatgttttgactccgtggaaatcagcggcgttg |
| | | | aagaccgctttaatgcatctctgggcacctaccacgatctactcaagatc |
| | | | atcaaggataaggacttcctggacaacgaagaaaatgaggatatcttgga |
| | | | agatattgtgctgactttaacgttgttcgaggatcgtgagatgattgaag |
| | | | aacgcctcaaaacgtacgcacatctgtttgacgataaggtgatgaaacag |
| | | | ctaaagcgccgtcgttacaccggttggggtcgtctgtctagaaagctgat |
| | | | taacggcattcgtgacaaacaatctggcaagaccatcctggacttcctca |
| | | | aatccgacggcttcgcaaatcgtaatttcatgcaactgatccatgatgat |
| | | | agcctgacctttaaagaagatattcagaaggctcaagtgtccggccaggg |
| | | | tgactcgctgcatgagcatatcgcgaatctggctggctcccctgcgataa |
| | | | aaaaaggtattctccaaacggtgaaagttgtggatgaactggttaaagtc |
| | | | atgggtcgtcataaaccggaaaacatcgtgattgagatggcacgtgagaa |
| | | | ccagaccacccagaaaggtcaaaaaaacagtcgtgagcgtatgaaacgta |
| | | | tcgaagaggggattaaggagttaggcagccagattctgaaggagcacccg |
| | | | gttgagaacacccagctgcaaaacgagaagctctacctgtattacctgca |
| | | | aaacggccgtgacatgtatgttgaccaggagctggacatcaatcgtctca |
| | | | gcgactacgacgtggatgctatcgtgccgcagagctttctaaaagatgat |
| | | | tccatcgacaataaagtgctcacccgtagcgacaagaaccgtggtaaaag |
| | | | cgacaacgtgccgagcgaggaagtagttaagaaaatgaaaaactactggc |
| | | | gtcagctattgaacgccaagcttatcacccagcgtaagtttgacaacttg |
| | | | acgaaggcagagcgtggcggcctgagcgagctagacaaggcgggttttat |
| | | | taaacgccagctggtggaaactcgccagattaccaaacacgttgcgcaaa |
| | | | tcttggactcacgcatgaatacgaagtacgacgaaaacgataaattgatc |
| | | | cgcgaggtgaaggtcataaccctgaaatctaaactggttagcgatttccg |
| | | | gaaggactttcaattttataaagtccgggagattaacaactatcaccatg |
| | | | cacacgacgcttacttaaatgcggttgttggcacggccctgatcaagaag |
| | | | tacccgaaactggaaagcgagtttgtttatggtgactacaaggtgtatga |
| | | | cgttcgcaagatgattgcaaaatccgaacaagagattggtaaggcgacgg |
| | | | ctaagtattttttctacagcaatatcatgaacttcttcaagactgaaatc |
| | | | accctggcgaacggtgaaattcgtaaacgtccgttgatcgaaaccaacgg |
| | | | tgaaaccggtgaaatcgtttgggataaaggtagggattttgcgaccgttc |
| | | | gtaaggtcctgagcatgccgcaggttaacatcgtgaaaaagaccgaagtg |
| | | | cagaccggcggtttctccaaggagagtatcttaccgaagcgcaacagtga |
| | | | caagctgatcgcgcgtaaaaaagactgggatccgaaaaaatacggcggct |
| | | | ttgactccccgaccgtcgcgtacagcgtactggtcgtggctaaagtggag |
| | | | aaaggcaaatcgaaaaagctgaaatccgttaaagagttgctgggtattac |
| | | | gattatggaacgttcgagcttcgagaaaaatccgatcgacttcctggagg |
| | | | ccaaaggctacaaggaagttaaaaaagacctgatcattaaactgccgaag |
| | | | tacagcctgttcgagctcgagaacggtcgcaaacgcatgctggcgagcgc |
| | | | gggtgagctccaaaaaggcaacgaattggctctgccgagcaagtacgtga |
| | | | acttcctgtatctggcctcccattatgaaaaactgaagggtagcccggaa |
| | | | gataacgaacaaaagcagctgttcgtggaacaacataaacactatctgga |
| | | | cgagattattgagcaaatcagcgagttcagcaaacgcgtgattctggcgg |
| | | | atgctaatctggacaaggttctttcagcctataataaacaccgtgataag |
| | | | ccgatccgtgaacaggcagaaaatattattcacctgttcaccctgacgaa |
| | | | cctgggtgcgccagcagcgtttaaatacttcgacacaacgatcgaccgga |
| | | | agcgttataccagcaccaaagaggttcttgacgccaccttgattcaccaa |
| | | | agcatcacgggtctgtatgaaacccgtattgacttgagccagttgggcgg |
| | | | agattcagggggaagtggcggaagcggtgggtctacgaacctgagcgaca |
| | | | ttattgaaaaagaaactggtaaacagttggtgatccaagaatccattctg |
| | | | atgttaccggaggaagtggaagaggtgatcggcaacaagccggagtccga |
| | | | catcctggttcacaccgcgtatgatgagtcgaccgatgagaatgttatgc |
| | | | tgctgaccagcgacgccccagagtacaaaccgtgggcgctcgtcatccag |
| | | | gatagcaatggcgaaaacaagatcaagatgttgtctggcggtagcatggc |
| | | | tccgaaaaagaagcgtaaagtaggtattcatggtgttccggcatcaggtt |
| | | | aa |
| 20 | Base editor | eA3A- | atgggaagtcatcaccaccaccaccattcaggtgagaacctgtacttcca |
| | | dCasPhi-2 | gagcatggctccgaaaaagaaacgtaaggtgggtatccacggcgttccgg |
| | | | cgatggaagcaagtcccgcttcaggaccaaggcacctgatggacccacat |
| | | | attttcaccagtaattttaacaacggcatcggccgtcacaagacctacct |
| | | | gtgttatgaggtcgaacgtctggataatggcacttccgtgaaaatggatc |
| | | | agcatcgtggtttcctgcatggtcaggctaagaatctgctttgcggtttc |
| | | | tatggtcgccacgccgaactgcgttttttggacctggttccgtccctcca |
| | | | actggacccggcgcaaatctatcgtgttacctggtttatcagctggtcgc |
| | | | cgtgtttcagctggggttgcgcaggcgaagtacgtgcgttcttacaagag |
| | | | aacacccatgtgcgcttgcgcatcttcgccgcacgtatttacgactacga |
| | | | cccgctgtataaagaggcgttgcaaatgctgcgcgatgctggcgcacagg |
| | | | ttagcattatgacgtacgacgaatttaagcactgctgggataccttcgtg |
| | | | gatcaccagggctgcccgtttcagccgtgggatggtttggacgagcactc |
| | | | tcaggcgctgagcggtcgtctcagagcgattctgcaaaaccagggaaact |
| | | | ctggaagtgagacacccgggacttcagaatccgcgaccccggagtctgga |
| | | | atgccaaaacccgctgtagagtcagaattttcaaaagttttgaaaaagca |
| | | | ctttccgggagaacgtttccgctcgagctatatgaaaagaggcggtaaga |
| | | | tcctggctgcccaaggcgaagaggcagttgttgcgtatctacagggcaag |
| | | | tccgaggaggagcctccgaacttccagccaccggcgaagtgccacgtggt |
| | | | gaccaaatcccgtgacttcgcggaatggccgatcatgaaggcgtcggaag |
| | | | cgattcaacgttatatctatgcactgtcgaccaccgaacgcgcggcctgt |
| | | | aaaccgggcaaaagtagtgagagccatgcagcgtggttcgccgctaccgg |
| | | | tgtcagcaaccacggctacagccacgttcagggtctgaacctgatttttg |
| | | | atcataccttgggccgttacgatggtgttctgaaaaaggtccaactgcgc |
| | | | aacgaaaaggcgagagcgcgtttagagagcattaacgcgtctagagcaga |
| | | | tgaaggtttaccggagattaaagccgaggaggaagaagtggctacgaacg |
| | | | aaaccggccacctgttgcaaccgccgggtatcaatccgagcttttacgtg |
| | | | tatcagaccattagcccgcaggcataccgtccgcgtgacgagatcgtgct |
| | | | gccgccggagtacgcaggttacgtacgtgacccgaatgctccgattccgc |
| | | | tgggtgtggtgcgtaaccgttgcgatatccaaaaagggtgcccgggctac |
| | | | atcccggaatggcagcgtgaagcaggtaccgcgatcagcccgaaaacagg |
| | | | caaggccgtaactgtgccgggcctgtctccgaagaaaaacaaacgtatgc |
| | | | gtaggtactggcgctctgagaaggagaaagctcaagatgcgctgttggtt |
| | | | accgtacgcattggtaccgattgggttgtcatcgacgtgcgcggtctgtt |
| | | | gcgcaacgcccgttggcgcaccatcgctcccaaggacatcagcctgaatg |
| | | | cgctcctcgatttgtttaccggtgatccggtgatcgatgtgcgtcgcaat |
| | | | attgttacctttacgtacaccctcgacgcctgtggtacctacgcccgtaa |
| | | | atggaccttgaaaggcaaacagacgaaagcgaccctggacaaactgacgg |
| | | | ccactcagacggttgcgttggttgcgattgcgcttggtcagaccaacccg |
| | | | atttccgctggcattagccgtgttacacaagaaaatggtgcactgcaatg |
| | | | cgaaccgctggaccggttcaccctgccggatgacctgttgaaggacatca |
| | | | gcgcatatcgcattgcttgggacagaaacgaagaggaactacgcgctcgc |
| | | | agcgtcgaggccctgccggaggcgcaacaagcggaggtccgtgcgctgga |
| | | | cggggtgtccaaagaaaccgcgcgtacccaactgtgcgcagatttcggcc |
| | | | tggacccgaaacgtctgccgtgggataaaatgagctccaatactaccttt |
| | | | atcagcgaagccttgctgtctaacagcgtgagccgtgatcaggttttttt |
| | | | cactccggctccgaagaagggtgctaagaaaaaagcgcctgttgaagtta |
| | | | tgcgtaaagaccgcacttgggcacgtgcgtacaagccgcgtctgagcgtc |
| | | | gaagctcagaaactgaagaacgaagccctgtgggcacttaagcgcacctc |
| | | | cccggagtatttgaagctgagccgtcgcaaagaggagctgtgtcgtcgta |
| | | | gcatcaactatgttatcgaaaagacccgtcgccgtacgcaatgtcagatt |
| | | | gtgatcccggtgatcgaggatctgaatgttcgcttctttcatggttctgg |
| | | | taagagactgcccggttgggataatttcttcaccgctaaaaaagagaacc |
| | | | gttggttcatccagggcctgcacaaagcgtttagcgatctgcgtacgcac |
| | | | cgttccttctatgtttttgaagttcgtccggagcgcacctctattacctg |
| | | | tccaaagtgcggtcattgcgaggtgggtaatcgtgacggtgaggcgtttc |
| | | | agtgcctgagctgcggcaagacgtgcaacgcagatcttgacgtcgcgacc |
| | | | cataatctgacccaagtggcgttgaccggtaagaccatgccgaagcgcga |
| | | | ggagccgcgtgacgctcagggtacagcgccagcgcgcaagactaagaagg |
| | | | cgagcaaatcaaaggcgccaccggcggagcgcgaagaccagaccccggcg |
| | | | caagagccaagccagaccagttctggaggtagcgggggtagcggtggtag |
| | | | cactaacttaagtgatattatcgagaaagaaaccgggaaacagctggtga |
| | | | tccaagaaagcattctgatgctgcccgaagaagttgaagaagtgattggt |
| | | | aacaagcctgagtcggacatcttagtgcacaccgcgtacgatgaaagcac |
| | | | ggatgagaatgtgatgctgctcacctctgatgcccccgaatacaagccgt |
| | | | gggctctggtgatccaggactctaacggagaaaacaaaattaagatgctc |
| | | | tctggcggctccatggcgcctaaaaaaaaacgcaaagtggggattcacgg |
| | | | ggttccggcgtaa |
| 21 | Base editor | eA3A- | atgggaagtcatcaccaccaccaccattcaggtgagaacctgtacttcca |
| | | dLbCas12a | gagcatggctccgaaaaagaaacgtaaggtgggtatccacggcgttccgg |
| | | | cgatggaagcaagtcccgcttcaggaccaaggcacctgatggacccacat |
| | | | attttcaccagtaattttaacaacggcatcggccgtcacaagacctacct |
| | | | gtgttatgaggtcgaacgtctggataatggcacttccgtgaaaatggatc |
| | | | agcatcgtggtttcctgcatggtcaggctaagaatctgctttgcggtttc |
| | | | tatggtcgccacgccgaactgcgttttttggacctggttccgtccctcca |
| | | | actggacccggcgcaaatctatcgtgttacctggtttatcagctggtcgc |
| | | | cgtgtttcagctggggttgcgcaggcgaagtacgtgcgttcttacaagag |
| | | | aacacccatgtgcgcttgcgcatcttcgccgcacgtatttacgactacga |
| | | | cccgctgtataaagaggcgttgcaaatgctgcgcgatgctggcgcacagg |
| | | | ttagcattatgacgtacgacgaatttaagcactgctgggataccttcgtg |
| | | | gatcaccagggctgcccgtttcagccgtgggatggtttggacgagcactc |
| | | | tcaggcgctgagcggtcgtctcagagcgattctgcaaaaccagggaaact |
| | | | ctggaagtgagacacccgggacttcagaatccgcgaccccggagtctgga |
| | | | atgtcaaaactagaaaagtttacaaattgttactccctgtcgaaaaccct |
| | | | gagattcaaagcgatcccggtgggtaaaactcaagagaacattgataaca |
| | | | aacgtctgctggttgaggacgaaaagcgcgcggaggattacaaaggcgtt |
| | | | aaaaagctgttggaccgttattacctgtcctttattaatgatgttctgca |
| | | | cagcattaaattaaagaacctaaacaattacatctccctcttccgtaaaa |
| | | | agacccgcaccgaaaaggaaaataaagaactggagaaccttgagattaat |
| | | | ctgcgtaaagaaatcgctaaagcgtttaaaggcaacgagggttataaatc |
| | | | cttatttaaaaaagacatcatcgaaacgattctcccagaattcctggacg |
| | | | acaaagacgagatcgcgctggttaattccttcaacggctttaccaccgca |
| | | | ttcaccggcttttttgataaccgcgaaaacatgttctctgaagaggccaa |
| | | | aagcacctcaattgcgtttcgctgcattaacgaaaatctgacgcgttata |
| | | | tctctaacatggatatcttcgagaaggttgacgctatctttgataaacat |
| | | | gaagtccaggagatcaaggagaagatcctgaacagcgactacgacgtgga |
| | | | ggatttcttcgagggtgagttcttcaattttgttttgacccaagaaggca |
| | | | tcgatgtctacaatgcgataattggtggttttgttaccgagagcggggag |
| | | | aaaattaagggtctgaacgaatatatcaacttgtacaaccagaaaaccaa |
| | | | acaaaaactgccgaagtttaagccgctgtacaagcaagtcttgtcggatc |
| | | | gtgaatcgctgagcttttacggcgaaggctacaccagcgatgaggaggtg |
| | | | ttggaggtgttccgcaatacgttgaataagaactctgaaattttttccag |
| | | | cattaagaagctggaaaaactattcaagaacttcgacgagtatagcagcg |
| | | | caggtatctttgtgaaaaacggtccggctattagcaccatctcgaaagac |
| | | | attttcggcgaatggaatgttattcgtgataagtggaatgccgaatacga |
| | | | cgacattcatctgaagaaaaaggcggttgtaactgaaaagtacgaggacg |
| | | | atcgtcgtaagtcgttcaaaaagatcggctccttctctctggagcaactg |
| | | | caagagtacgccgatgccgatttgtcagtcgtggagaagctgaaagagat |
| | | | catcattcaaaaagttgacgaaatctataaggtctatggtagcagtgaaa |
| | | | aactgtttgacgcggacttcgtgctggagaaaagcctgaagaaaaatgac |
| | | | gccgtggtggcaatcatgaaagatcttctggatagcgtcaagtctttcga |
| | | | aaactacatcaaagcgtttttcggtgaaggcaaggaaacgaatcgtgacg |
| | | | agagcttctatggtgatttcgtcctagcctatgatatcctgcttaaggtg |
| | | | gatcacatctatgatgccatccgcaactatgttacccagaaaccgtactc |
| | | | aaaggacaagttcaagttatacttccagaatccgcagtttatgggtggct |
| | | | gggataaagataaggaaaccgactatcgcgccaccattttacgttacggt |
| | | | agtaagtactatctggcaatcatggataaaaaatatgcgaaatgcctgca |
| | | | aaagatcgacaaggacgatgttaacggcaattatgagaagatcaactaca |
| | | | aactgctgccgggcccaaataagatgctgccgaaggtgtttttcagcaaa |
| | | | aaatggatggcgtattacaatccgagcgaggacatccaaaagatctacaa |
| | | | aaacggtacgtttaagaagggtgatatgttcaacctcaacgattgtcata |
| | | | aacttattgacttcttcaaggatagcatatctcgttatccgaagtggagc |
| | | | aacgcgtatgactttaactttagcgaaaccgagaaatacaaagatatcgc |
| | | | gggtttctatcgtgaagttgaagaacagggttacaaagtgtccttcgagt |
| | | | ccgcgagcaagaaggaggtagacaagctggtcgaggagggtaagttgtac |
| | | | atgtttcagatttacaataaggacttttccgacaagtcccatggtacacc |
| | | | gaatttgcacaccatgtatttcaagcttctgttcgacgagaacaaccacg |
| | | | gccagatccgtctgagcggtggtgcagagctgttcatgcgtagagctagc |
| | | | ctgaagaaagaagagttggtcgttcatccggctaattcgccgatcgcgaa |
| | | | caaaaatccagataaccctaaaaagactacgaccctgagctatgatgtgt |
| | | | ataaagacaaacgctttagcgaagaccagtatgagttacacattccgatc |
| | | | gctatcaacaagtgcccgaagaatattttcaagatcaacaccgaagtgcg |
| | | | tgttcttttgaagcacgatgacaacccgtatgtgattggtattgctcgtg |
| | | | gtgaacgtaatctgctgtacattgtggttgtggacggcaaaggaaacatc |
| | | | gtggaacagtacagcctgaacgaaatcattaacaatttcaacggcattcg |
| | | | cattaagacggattaccattctttgctggacaaaaaggagaaagagcgtt |
| | | | tcgaggcccgtcagaactggacctccatcgagaacattaaggagctgaag |
| | | | gcgggttacatcagccaggtggtgcacaaaatctgcgaactggtggagaa |
| | | | gtacgacgcggtgattgctttggcggatttgaactccggttttaagaaca |
| | | | gccgcgttaaagtggaaaagcaggtttatcagaaattcgaaaaaatgctg |
| | | | attgataaattgaattacatggttgacaaaaagtctaacccgtgtgcaac |
| | | | cggcggtgcgctgaagggctaccaaatcaccaataagtttgaaagcttta |
| | | | agagcatgagcacgcaaaatggcttcattttttacattccggcgtggctg |
| | | | accagcaagatcgatccgtcaaccggctttgttaatttactgaagacgaa |
| | | | gtatacctctattgccgactctaaaaaatttattagttccttcgatagaa |
| | | | tcatgtatgttccggaagaggacctgttcgaatttgcactggactacaag |
| | | | aactttagccgtaccgatgctgattatataaagaaatggaaactctacag |
| | | | ctatggcaaccgcatccgtatctttcgcaacccgaaaaaaaacaacgtgt |
| | | | tcgactgggaagaggtatgtctgacttcagcgtacaaggagcttttcaat |
| | | | aaatatggtatcaactaccagcagggtgatatccgtgcactgctttgcga |
| | | | gcaaagcgataaagcgttctattcaagcttcatggcgctgatgagcctga |
| | | | tgctgcaaatgcgcaacagcatcaccggtcgtaccgatgttgcatttctg |
| | | | atttctccagtaaaaaacagcgatggtatcttttacgacagccgtaatta |
| | | | cgaggcccaagaaaatgcgattttgccgaagaacgctgacgcgaatggtg |
| | | | catacaacattgcaaggaaagttctgtgggcaatcggccaatttaaaaaa |
| | | | gcggaagatgagaagctggacaaagtgaaaattgcgattagcaacaagga |
| | | | gtggctggagtatgcgcagacctccgttaaatcagggggaagtggcggaa |
| | | | gcggtgggtctacgaacctgagcgacattattgaaaaagaaactggtaaa |
| | | | cagttggtgatccaagaatccattctgatgttaccggaggaagtggaaga |
| | | | ggtgatcggcaacaagccggagtccgacatcctggttcacaccgcgtatg |
| | | | atgagtcgaccgatgagaatgttatgctgctgaccagcgacgccccagag |
| | | | tacaaaccgtgggcgctcgtcatccaggatagcaatggcgaaaacaagat |
| | | | caagatgttgtctggcggtagcatggctccgaaaaagaagcgtaaagtag |
| | | | gtattcatggtgttccggcatcaggttaa |
| 22 | Base editor | evoAPOBEC1- | atgggaagtcatcaccaccaccaccattcaggtgagaacctgtacttcca |
| | | dCas9 | gagcatggctccgaaaaagaaacgtaaggtgggtatccacggcgttccgg |
| | | | cgatgtcaagtaaaacaggacccgttgctgtagatccgacgctgagacgt |
| | | | cgcatcgagccgcacgaattcgaggtgttttttgatccgcgtgaactgcg |
| | | | caaggagacgtgcctgctgtatgagatcaactggggtggccgtcacagca |
| | | | tttggcgtcacacctcccaaaacacgaacaaacacgtggaagtcaatttc |
| | | | atcgaaaaattcaccaccgaacgttacttctgcccgaacacccgttgttc |
| | | | catcacctggttcctgtcttggagcccgtgtggtgagtgcagccgcgcaa |
| | | | ttactgagttcctgagccgttacccgaatgttaccctgtttatctatatc |
| | | | gcccgtttgtaccatctggcgaacccgcgcaaccgtcagggtttacgcga |
| | | | cttaattagctctggcgttaccattcagattatgaccgagcaagagagcg |
| | | | gttattgctggcataactttgtaaactatagcccgagcaatgaaagccat |
| | | | tggcctcgctacccgcacctgtgggttcgtttgtacgtgctggaactgta |
| | | | ctgcatcatccttggtttgccaccgtgcttgaatattttgagacgtaagc |
| | | | agtcgcaactgaccagttttacgatcgcgctacagagctgtcattatcag |
| | | | cgtctcccgccacacattctgtgggctaccggcctgaaaagcggcggtag |
| | | | cagcggtggctccagcggctcggaaactccgggcacctccgagtcagcga |
| | | | ccccggaatctggaagtgagacacccgggacttcagaatccgcgaccccg |
| | | | gagtctggaatggataaaaagtattcaataggactagctattggcaccaa |
| | | | cagcgttggttgggcagttatcaccgatgaatataaggtcccgagcaaga |
| | | | agtttaaggtcctgggcaacaccgatcgccacagcataaaaaagaacctt |
| | | | attggtgcgctgctgttcgattctggtgagactgccgaagcgacccgtct |
| | | | gaagagaaccgcgcgtcgtagatacacccggcgcaagaaccgtatctgct |
| | | | atctgcaagaaattttcagcaacgagatggctaaggtcgacgactccttc |
| | | | tttcatcgcctggaggaaagctttctggttgaggaagacaaaaagcacga |
| | | | gcgtcatccgattttcggtaacattgttgatgaagttgcctaccacgaaa |
| | | | aataccccacaatttatcatctgcgtaaaaaactggtagactccaccgat |
| | | | aaggcggatttacgccttatctatttggcgctggcgcatatgattaagtt |
| | | | ccgcggtcattttttgatcgagggcgacctgaatccggataacagcgacg |
| | | | ttgacaaactgtttatccagctggtgcagacctataaccagttgttcgaa |
| | | | gagaacccgattaatgcgtctggcgtggacgcgaaggcgatcctgagcgc |
| | | | tcgtctgtccaagtcacgccgcctggagaacttgatcgcgcaactgccgg |
| | | | gtgagaagaaaaacggcttgttcggcaatctgattgctctgtccctaggt |
| | | | ttgaccccgaatttcaaatctaattttgatctggcggaagatgcgaaatt |
| | | | acagttgagcaaggacacttacgatgacgatctggacaacctgctcgctc |
| | | | agatcggcgatcagtatgccgatcttttcctggccgcgaagaacctgagc |
| | | | gatgccatcttgctatcggacatcttacgtgttaacaccgaaatcaccaa |
| | | | agctccgctaagcgcgtccatgattaaacgttacgatgaacaccaccagg |
| | | | atctgacgctgctgaaagcgttggtgcgccagcaactgccggaaaaatac |
| | | | aaggaaattttcttcgaccaaagcaagaacggttatgcaggctatatcga |
| | | | cggtggtgcgtcgcaagaagaattttataaattcatcaaaccgattttgg |
| | | | agaagatggatggtaccgaggagctgctggtaaaattgaatcgggaagac |
| | | | ctcttgcgaaagcagcgtactttcgataatggtagcatcccgcaccagat |
| | | | tcatctgggcgaacttcacgcaatcctacgtcgtcaagaggatttctacc |
| | | | cgtttctgaaggataaccgtgaaaagatcgagaagattctgacctttcgt |
| | | | attccgtattatgtgggtccgctggcgagaggcaacagccgttttgcgtg |
| | | | gatgacccgcaagtcggaagagactatcacgccgtggaatttcgaggagg |
| | | | tcgtggacaagggtgcatcagcgcaatcttttattgaacgtatgaccaat |
| | | | tttgataaaaaccttccaaatgaaaaagtgttgccgaagcacagcttgct |
| | | | gtacgagtatttcaccgtttacaatgaactgacgaaagtcaagtacgtta |
| | | | ccgaaggtatgcgtaagccagcctttctgtctggggagcaaaaaaaagcc |
| | | | attgtggatctgctgttcaagaccaatcgcaaggttaccgtcaagcaact |
| | | | gaaagaggattacttcaaaaaaatcgaatgttttgactccgtggaaatca |
| | | | gcggcgttgaagaccgctttaatgcatctctgggcacctaccacgatcta |
| | | | ctcaagatcatcaaggataaggacttcctggacaacgaagaaaatgagga |
| | | | tatcttggaagatattgtgctgactttaacgttgttcgaggatcgtgaga |
| | | | tgattgaagaacgcctcaaaacgtacgcacatctgtttgacgataaggtg |
| | | | atgaaacagctaaagcgccgtcgttacaccggttggggtcgtctgtctag |
| | | | aaagctgattaacggcattcgtgacaaacaatctggcaagaccatcctgg |
| | | | acttcctcaaatccgacggcttcgcaaatcgtaatttcatgcaactgatc |
| | | | catgatgatagcctgacctttaaagaagatattcagaaggctcaagtgtc |
| | | | cggccagggtgactcgctgcatgagcatatcgcgaatctggctggctccc |
| | | | ctgcgataaaaaaaggtattctccaaacggtgaaagttgtggatgaactg |
| | | | gttaaagtcatgggtcgtcataaaccggaaaacatcgtgattgagatggc |
| | | | acgtgagaaccagaccacccagaaaggtcaaaaaaacagtcgtgagcgta |
| | | | tgaaacgtatcgaagaggggattaaggagttaggcagccagattctgaag |
| | | | gagcacccggttgagaacacccagctgcaaaacgagaagctctacctgta |
| | | | ttacctgcaaaacggccgtgacatgtatgttgaccaggagctggacatca |
| | | | atcgtctcagcgactacgacgtggatgctatcgtgccgcagagctttcta |
| | | | aaagatgattccatcgacaataaagtgctcacccgtagcgacaagaaccg |
| | | | tggtaaaagcgacaacgtgccgagcgaggaagtagttaagaaaatgaaaa |
| | | | actactggcgtcagctattgaacgccaagcttatcacccagcgtaagttt |
| | | | gacaacttgacgaaggcagagcgtggcggcctgagcgagctagacaaggc |
| | | | gggttttattaaacgccagctggtggaaactcgccagattaccaaacacg |
| | | | ttgcgcaaatcttggactcacgcatgaatacgaagtacgacgaaaacgat |
| | | | aaattgatccgcgaggtgaaggtcataaccctgaaatctaaactggttag |
| | | | cgatttccggaaggactttcaattttataaagtccgggagattaacaact |
| | | | atcaccatgcacacgacgcttacttaaatgcggttgttggcacggccctg |
| | | | atcaagaagtacccgaaactggaaagcgagtttgtttatggtgactacaa |
| | | | ggtgtatgacgttcgcaagatgattgcaaaatccgaacaagagattggta |
| | | | aggcgacggctaagtattttttctacagcaatatcatgaacttcttcaag |
| | | | actgaaatcaccctggcgaacggtgaaattcgtaaacgtccgttgatcga |
| | | | aaccaacggtgaaaccggtgaaatcgtttgggataaaggtagggattttg |
| | | | cgaccgttcgtaaggtcctgagcatgccgcaggttaacatcgtgaaaaag |
| | | | accgaagtgcagaccggcggtttctccaaggagagtatcttaccgaagcg |
| | | | caacagtgacaagctgatcgcgcgtaaaaaagactgggatccgaaaaaat |
| | | | acggcggctttgactccccgaccgtcgcgtacagcgtactggtcgtggct |
| | | | aaagtggagaaaggcaaatcgaaaaagctgaaatccgttaaagagttgct |
| | | | gggtattacgattatggaacgttcgagcttcgagaaaaatccgatcgact |
| | | | tcctggaggccaaaggctacaaggaagttaaaaaagacctgatcattaaa |
| | | | ctgccgaagtacagcctgttcgagctcgagaacggtcgcaaacgcatgct |
| | | | ggcgagcgcgggtgagctccaaaaaggcaacgaattggctctgccgagca |
| | | | agtacgtgaacttcctgtatctggcctcccattatgaaaaactgaagggt |
| | | | agcccggaagataacgaacaaaagcagctgttcgtggaacaacataaaca |
| | | | ctatctggacgagattattgagcaaatcagcgagttcagcaaacgcgtga |
| | | | ttctggcggatgctaatctggacaaggttctttcagcctataataaacac |
| | | | cgtgataagccgatccgtgaacaggcagaaaatattattcacctgttcac |
| | | | cctgacgaacctgggtgcgccagcagcgtttaaatacttcgacacaacga |
| | | | tcgaccggaagcgttataccagcaccaaagaggttcttgacgccaccttg |
| | | | attcaccaaagcatcacgggtctgtatgaaacccgtattgacttgagcca |
| | | | gttgggcggagattcagggggaagtggcggaagcggtgggtctacgaacc |
| | | | tgagcgacattattgaaaaagaaactggtaaacagttggtgatccaagaa |
| | | | tccattctgatgttaccggaggaagtggaagaggtgatcggcaacaagcc |
| | | | ggagtccgacatcctggttcacaccgcgtatgatgagtcgaccgatgaga |
| | | | atgttatgctgctgaccagcgacgccccagagtacaaaccgtgggcgctc |
| | | | gtcatccaggatagcaatggcgaaaacaagatcaagatgttgtctggcgg |
| | | | tagcatggctccgaaaaagaagcgtaaagtaggtattcatggtgttccgg |
| | | | catcaggttaa |
| 23 | Base editor | evo APOBEC1- | atgggaagtcatcaccaccaccaccattcaggtgagaacctgtacttcca |
| | | dCasPhi-2 | gagcatggctccgaaaaagaaacgtaaggtgggtatccacggcgttccgg |
| | | | cgatgtcaagtaaaacaggacccgttgctgtagatccgacgctgagacgt |
| | | | cgcatcgagccgcacgaattcgaggtgttttttgatccgcgtgaactgcg |
| | | | caaggagacgtgcctgctgtatgagatcaactggggtggccgtcacagca |
| | | | tttggcgtcacacctcccaaaacacgaacaaacacgtggaagtcaatttc |
| | | | atcgaaaaattcaccaccgaacgttacttctgcccgaacacccgttgttc |
| | | | catcacctggttcctgtcttggagcccgtgtggtgagtgcagccgcgcaa |
| | | | ttactgagttcctgagccgttacccgaatgttaccctgtttatctatatc |
| | | | gcccgtttgtaccatctggcgaacccgcgcaaccgtcagggtttacgcga |
| | | | cttaattagctctggcgttaccattcagattatgaccgagcaagagagcg |
| | | | gttattgctggcataactttgtaaactatagcccgagcaatgaaagccat |
| | | | tggcctcgctacccgcacctgtgggttcgtttgtacgtgctggaactgta |
| | | | ctgcatcatccttggtttgccaccgtgcttgaatattttgagacgtaagc |
| | | | agtcgcaactgaccagttttacgatcgcgctacagagctgtcattatcag |
| | | | cgtctcccgccacacattctgtgggctaccggcctgaaaagcggcggtag |
| | | | cagcggtggctccagcggctcggaaactccgggcacctccgagtcagcga |
| | | | ccccggaatctggaagtgagacacccgggacttcagaatccgcgaccccg |
| | | | gagtctggaatgccaaaacccgctgtagagtcagaattttcaaaagtttt |
| | | | gaaaaagcactttccgggagaacgtttccgctcgagctatatgaaaagag |
| | | | gcggtaagatcctggctgcccaaggcgaagaggcagttgttgcgtatcta |
| | | | cagggcaagtccgaggaggagcctccgaacttccagccaccggcgaagtg |
| | | | ccacgtggtgaccaaatcccgtgacttcgcggaatggccgatcatgaagg |
| | | | cgtcggaagcgattcaacgttatatctatgcactgtcgaccaccgaacgc |
| | | | gcggcctgtaaaccgggcaaaagtagtgagagccatgcagcgtggttcgc |
| | | | cgctaccggtgtcagcaaccacggctacagccacgttcagggtctgaacc |
| | | | tgatttttgatcataccttgggccgttacgatggtgttctgaaaaaggtc |
| | | | caactgcgcaacgaaaaggcgagagcgcgtttagagagcattaacgcgtc |
| | | | tagagcagatgaaggtttaccggagattaaagccgaggaggaagaagtgg |
| | | | ctacgaacgaaaccggccacctgttgcaaccgccgggtatcaatccgagc |
| | | | ttttacgtgtatcagaccattagcccgcaggcataccgtccgcgtgacga |
| | | | gatcgtgctgccgccggagtacgcaggttacgtacgtgacccgaatgctc |
| | | | cgattccgctgggtgtggtgcgtaaccgttgcgatatccaaaaagggtgc |
| | | | ccgggctacatcccggaatggcagcgtgaagcaggtaccgcgatcagccc |
| | | | gaaaacaggcaaggccgtaactgtgccgggcctgtctccgaagaaaaaca |
| | | | aacgtatgcgtaggtactggcgctctgagaaggagaaagctcaagatgcg |
| | | | ctgttggttaccgtacgcattggtaccgattgggttgtcatcgacgtgcg |
| | | | cggtctgttgcgcaacgcccgttggcgcaccatcgctcccaaggacatca |
| | | | gcctgaatgcgctcctcgatttgtttaccggtgatccggtgatcgatgtg |
| | | | cgtcgcaatattgttacctttacgtacaccctcgacgcctgtggtaccta |
| | | | cgcccgtaaatggaccttgaaaggcaaacagacgaaagcgaccctggaca |
| | | | aactgacggccactcagacggttgcgttggttgcgattgcgcttggtcag |
| | | | accaacccgatttccgctggcattagccgtgttacacaagaaaatggtgc |
| | | | actgcaatgcgaaccgctggaccggttcaccctgccggatgacctgttga |
| | | | aggacatcagcgcatatcgcattgcttgggacagaaacgaagaggaacta |
| | | | cgcgctcgcagcgtcgaggccctgccggaggcgcaacaagcggaggtccg |
| | | | tgcgctggacggggtgtccaaagaaaccgcgcgtacccaactgtgcgcag |
| | | | atttcggcctggacccgaaacgtctgccgtgggataaaatgagctccaat |
| | | | actacctttatcagcgaagccttgctgtctaacagcgtgagccgtgatca |
| | | | ggtttttttcactccggctccgaagaagggtgctaagaaaaaagcgcctg |
| | | | ttgaagttatgcgtaaagaccgcacttgggcacgtgcgtacaagccgcgt |
| | | | ctgagcgtcgaagctcagaaactgaagaacgaagccctgtgggcacttaa |
| | | | gcgcacctccccggagtatttgaagctgagccgtcgcaaagaggagctgt |
| | | | gtcgtcgtagcatcaactatgttatcgaaaagacccgtcgccgtacgcaa |
| | | | tgtcagattgtgatcccggtgatcgaggatctgaatgttcgcttctttca |
| | | | tggttctggtaagagactgcccggttgggataatttcttcaccgctaaaa |
| | | | aagagaaccgttggttcatccagggcctgcacaaagcgtttagcgatctg |
| | | | cgtacgcaccgttccttctatgtttttgaagttcgtccggagcgcacctc |
| | | | tattacctgtccaaagtgcggtcattgcgaggtgggtaatcgtgacggtg |
| | | | aggcgtttcagtgcctgagctgcggcaagacgtgcaacgcagatcttgac |
| | | | gtcgcgacccataatctgacccaagtggcgttgaccggtaagaccatgcc |
| | | | gaagcgcgaggagccgcgtgacgctcagggtacagcgccagcgcgcaaga |
| | | | ctaagaaggcgagcaaatcaaaggcgccaccggcggagcgcgaagaccag |
| | | | accccggcgcaagagccaagccagaccagttctggaggtagcgggggtag |
| | | | cggtggtagcactaacttaagtgatattatcgagaaagaaaccgggaaac |
| | | | agctggtgatccaagaaagcattctgatgctgcccgaagaagttgaagaa |
| | | | gtgattggtaacaagcctgagtcggacatcttagtgcacaccgcgtacga |
| | | | tgaaagcacggatgagaatgtgatgctgctcacctctgatgcccccgaat |
| | | | acaagccgtgggctctggtgatccaggactctaacggagaaaacaaaatt |
| | | | aagatgctctctggcggctccatggcgcctaaaaaaaaacgcaaagtggg |
| | | | gattcacggggttccggcgtaa |
| 24 | Base editor | evo APOBEC1- | atgggaagtcatcaccaccaccaccattcaggtgagaacctgtacttcca |
| | | dLbCas12a | gagcatggctccgaaaaagaaacgtaaggtgggtatccacggcgttccgg |
| | | | cgatgtcaagtaaaacaggacccgttgctgtagatccgacgctgagacgt |
| | | | cgcatcgagccgcacgaattcgaggtgttttttgatccgcgtgaactgcg |
| | | | caaggagacgtgcctgctgtatgagatcaactggggtggccgtcacagca |
| | | | tttggcgtcacacctcccaaaacacgaacaaacacgtggaagtcaatttc |
| | | | atcgaaaaattcaccaccgaacgttacttctgcccgaacacccgttgttc |
| | | | catcacctggttcctgtcttggagcccgtgtggtgagtgcagccgcgcaa |
| | | | ttactgagttcctgagccgttacccgaatgttaccctgtttatctatatc |
| | | | gcccgtttgtaccatctggcgaacccgcgcaaccgtcagggtttacgcga |
| | | | cttaattagctctggcgttaccattcagattatgaccgagcaagagagcg |
| | | | gttattgctggcataactttgtaaactatagcccgagcaatgaaagccat |
| | | | tggcctcgctacccgcacctgtgggttcgtttgtacgtgctggaactgta |
| | | | ctgcatcatccttggtttgccaccgtgcttgaatattttgagacgtaagc |
| | | | agtcgcaactgaccagttttacgatcgcgctacagagctgtcattatcag |
| | | | cgtctcccgccacacattctgtgggctaccggcctgaaaagcggcggtag |
| | | | cagcggtggctccagcggctcggaaactccgggcacctccgagtcagcga |
| | | | ccccggaatctggaagtgagacacccgggacttcagaatccgcgaccccg |
| | | | gagtctggaatgtcaaaactagaaaagtttacaaattgttactccctgtc |
| | | | gaaaaccctgagattcaaagcgatcccggtgggtaaaactcaagagaaca |
| | | | ttgataacaaacgtctgctggttgaggacgaaaagcgcgcggaggattac |
| | | | aaaggcgttaaaaagctgttggaccgttattacctgtcctttattaatga |
| | | | tgttctgcacagcattaaattaaagaacctaaacaattacatctccctct |
| | | | tccgtaaaaagacccgcaccgaaaaggaaaataaagaactggagaacctt |
| | | | gagattaatctgcgtaaagaaatcgctaaagcgtttaaaggcaacgaggg |
| | | | ttataaatccttatttaaaaaagacatcatcgaaacgattctcccagaat |
| | | | tcctggacgacaaagacgagatcgcgctggttaattccttcaacggcttt |
| | | | accaccgcattcaccggcttttttgataaccgcgaaaacatgttctctga |
| | | | agaggccaaaagcacctcaattgcgtttcgctgcattaacgaaaatctga |
| | | | cgcgttatatctctaacatggatatcttcgagaaggttgacgctatcttt |
| | | | gataaacatgaagtccaggagatcaaggagaagatcctgaacagcgacta |
| | | | cgacgtggaggatttcttcgagggtgagttcttcaattttgttttgaccc |
| | | | aagaaggcatcgatgtctacaatgcgataattggtggttttgttaccgag |
| | | | agcggggagaaaattaagggtctgaacgaatatatcaacttgtacaacca |
| | | | gaaaaccaaacaaaaactgccgaagtttaagccgctgtacaagcaagtct |
| | | | tgtcggatcgtgaatcgctgagcttttacggcgaaggctacaccagcgat |
| | | | gaggaggtgttggaggtgttccgcaatacgttgaataagaactctgaaat |
| | | | tttttccagcattaagaagctggaaaaactattcaagaacttcgacgagt |
| | | | atagcagcgcaggtatctttgtgaaaaacggtccggctattagcaccatc |
| | | | tcgaaagacattttcggcgaatggaatgttattcgtgataagtggaatgc |
| | | | cgaatacgacgacattcatctgaagaaaaaggcggttgtaactgaaaagt |
| | | | acgaggacgatcgtcgtaagtcgttcaaaaagatcggctccttctctctg |
| | | | gagcaactgcaagagtacgccgatgccgatttgtcagtcgtggagaagct |
| | | | gaaagagatcatcattcaaaaagttgacgaaatctataaggtctatggta |
| | | | gcagtgaaaaactgtttgacgcggacttcgtgctggagaaaagcctgaag |
| | | | aaaaatgacgccgtggtggcaatcatgaaagatcttctggatagcgtcaa |
| | | | gtctttcgaaaactacatcaaagcgtttttcggtgaaggcaaggaaacga |
| | | | atcgtgacgagagcttctatggtgatttcgtcctagcctatgatatcctg |
| | | | cttaaggtggatcacatctatgatgccatccgcaactatgttacccagaa |
| | | | accgtactcaaaggacaagttcaagttatacttccagaatccgcagttta |
| | | | tgggtggctgggataaagataaggaaaccgactatcgcgccaccatttta |
| | | | cgttacggtagtaagtactatctggcaatcatggataaaaaatatgcgaa |
| | | | atgcctgcaaaagatcgacaaggacgatgttaacggcaattatgagaaga |
| | | | tcaactacaaactgctgccgggcccaaataagatgctgccgaaggtgttt |
| | | | ttcagcaaaaaatggatggcgtattacaatccgagcgaggacatccaaaa |
| | | | gatctacaaaaacggtacgtttaagaagggtgatatgttcaacctcaacg |
| | | | attgtcataaacttattgacttcttcaaggatagcatatctcgttatccg |
| | | | aagtggagcaacgcgtatgactttaactttagcgaaaccgagaaatacaa |
| | | | agatatcgcgggtttctatcgtgaagttgaagaacagggttacaaagtgt |
| | | | ccttcgagtccgcgagcaagaaggaggtagacaagctggtcgaggagggt |
| | | | aagttgtacatgtttcagatttacaataaggacttttccgacaagtccca |
| | | | tggtacaccgaatttgcacaccatgtatttcaagcttctgttcgacgaga |
| | | | acaaccacggccagatccgtctgagcggtggtgcagagctgttcatgcgt |
| | | | agagctagcctgaagaaagaagagttggtcgttcatccggctaattcgcc |
| | | | gatcgcgaacaaaaatccagataaccctaaaaagactacgaccctgagct |
| | | | atgatgtgtataaagacaaacgctttagcgaagaccagtatgagttacac |
| | | | attccgatcgctatcaacaagtgcccgaagaatattttcaagatcaacac |
| | | | cgaagtgcgtgttcttttgaagcacgatgacaacccgtatgtgattggta |
| | | | ttgctcgtggtgaacgtaatctgctgtacattgtggttgtggacggcaaa |
| | | | ggaaacatcgtggaacagtacagcctgaacgaaatcattaacaatttcaa |
| | | | cggcattcgcattaagacggattaccattctttgctggacaaaaaggaga |
| | | | aagagcgtttcgaggcccgtcagaactggacctccatcgagaacattaag |
| | | | gagctgaaggcgggttacatcagccaggtggtgcacaaaatctgcgaact |
| | | | ggtggagaagtacgacgcggtgattgctttggcggatttgaactccggtt |
| | | | ttaagaacagccgcgttaaagtggaaaagcaggtttatcagaaattcgaa |
| | | | aaaatgctgattgataaattgaattacatggttgacaaaaagtctaaccc |
| | | | gtgtgcaaccggcggtgcgctgaagggctaccaaatcaccaataagtttg |
| | | | aaagctttaagagcatgagcacgcaaaatggcttcattttttacattccg |
| | | | gcgtggctgaccagcaagatcgatccgtcaaccggctttgttaatttact |
| | | | gaagacgaagtatacctctattgccgactctaaaaaatttattagttcct |
| | | | tcgatagaatcatgtatgttccggaagaggacctgttcgaatttgcactg |
| | | | gactacaagaactttagccgtaccgatgctgattatataaagaaatggaa |
| | | | actctacagctatggcaaccgcatccgtatctttcgcaacccgaaaaaaa |
| | | | acaacgtgttcgactgggaagaggtatgtctgacttcagcgtacaaggag |
| | | | cttttcaataaatatggtatcaactaccagcagggtgatatccgtgcact |
| | | | gctttgcgagcaaagcgataaagcgttctattcaagcttcatggcgctga |
| | | | tgagcctgatgctgcaaatgcgcaacagcatcaccggtcgtaccgatgtt |
| | | | gcatttctgatttctccagtaaaaaacagcgatggtatcttttacgacag |
| | | | ccgtaattacgaggcccaagaaaatgcgattttgccgaagaacgctgacg |
| | | | cgaatggtgcatacaacattgcaaggaaagttctgtgggcaatcggccaa |
| | | | tttaaaaaagcggaagatgagaagctggacaaagtgaaaattgcgattag |
| | | | caacaaggagtggctggagtatgcgcagacctccgttaaatcagggggaa |
| | | | gtggcggaagcggtgggtctacgaacctgagcgacattattgaaaaagaa |
| | | | actggtaaacagttggtgatccaagaatccattctgatgttaccggagga |
| | | | agtggaagaggtgatcggcaacaagccggagtccgacatcctggttcaca |
| | | | ccgcgtatgatgagtcgaccgatgagaatgttatgctgctgaccagcgac |
| | | | gccccagagtacaaaccgtgggcgctcgtcatccaggatagcaatggcga |
| | | | aaacaagatcaagatgttgtctggcggtagcatggctccgaaaaagaagc |
| | | | gtaaagtaggtattcatggtgttccggcatcaggttaa |
| 25 | Base editor | evoCDA1-dCas9 | atgggaagtcatcaccaccaccaccattcaggtgagaacctgtacttcca |
| | | | gagcatggctccgaaaaagaaacgtaaggtgggtatccacggcgttccgg |
| | | | cgatgtcaacagatgctgaatatgtaaggatacacgagaagctggacatc |
| | | | tacacctttaaaaagcagttctccaacaataaaaaatctgtcagccatcg |
| | | | ttgctacgtgttgtttgaactcaagcgtcgtggcgaacgccgtgcgtgct |
| | | | tttggggttacgctgtgaacaaaccgcagagcggtacggaacgtggtatt |
| | | | catgcggagatcttcagcatccgcaaggtggaagagtatctgcgcgataa |
| | | | cccgggtcaattcaccattaactggtacagcagctggtcaccgtgcgccg |
| | | | actgtgctgagaagattttggagtggtataaccaagaattgcgcggtaat |
| | | | ggccatactctgaagatctgggtgtgtaaactttattacgaaaagaacgc |
| | | | gcgtaatcaaattggtctgtggaatctgagagataacggcgttggtctga |
| | | | atgttatggtttccgagcactatcagtgctgccgtaaaatcttcattcag |
| | | | tccagccacaaccaactgaacgagaaccgttggctggaaaagaccctgaa |
| | | | gcgcgcggagaaacgtcgtagcgagctgagcattatgtttcaggttaaaa |
| | | | tcttacacaccaccaaatcgccggcagtctctggcggttcctcgggcggc |
| | | | agctctggctccgaaaccccaggtacgagcgagagcgcaaccccggaatc |
| | | | tggaagtgagacacccgggacttcagaatccgcgaccccggagtctggaa |
| | | | tggataaaaagtattcaataggactagctattggcaccaacagcgttggt |
| | | | tgggcagttatcaccgatgaatataaggtcccgagcaagaagtttaaggt |
| | | | cctgggcaacaccgatcgccacagcataaaaaagaaccttattggtgcgc |
| | | | tgctgttcgattctggtgagactgccgaagcgacccgtctgaagagaacc |
| | | | gcgcgtcgtagatacacccggcgcaagaaccgtatctgctatctgcaaga |
| | | | aattttcagcaacgagatggctaaggtcgacgactccttctttcatcgcc |
| | | | tggaggaaagctttctggttgaggaagacaaaaagcacgagcgtcatccg |
| | | | attttcggtaacattgttgatgaagttgcctaccacgaaaaataccccac |
| | | | aatttatcatctgcgtaaaaaactggtagactccaccgataaggcggatt |
| | | | tacgccttatctatttggcgctggcgcatatgattaagttccgcggtcat |
| | | | tttttgatcgagggcgacctgaatccggataacagcgacgttgacaaact |
| | | | gtttatccagctggtgcagacctataaccagttgttcgaagagaacccga |
| | | | ttaatgcgtctggcgtggacgcgaaggcgatcctgagcgctcgtctgtcc |
| | | | aagtcacgccgcctggagaacttgatcgcgcaactgccgggtgagaagaa |
| | | | aaacggcttgttcggcaatctgattgctctgtccctaggtttgaccccga |
| | | | atttcaaatctaattttgatctggcggaagatgcgaaattacagttgagc |
| | | | aaggacacttacgatgacgatctggacaacctgctcgctcagatcggcga |
| | | | tcagtatgccgatcttttcctggccgcgaagaacctgagcgatgccatct |
| | | | tgctatcggacatcttacgtgttaacaccgaaatcaccaaagctccgcta |
| | | | agcgcgtccatgattaaacgttacgatgaacaccaccaggatctgacgct |
| | | | gctgaaagcgttggtgcgccagcaactgccggaaaaatacaaggaaattt |
| | | | tcttcgaccaaagcaagaacggttatgcaggctatatcgacggtggtgcg |
| | | | tcgcaagaagaattttataaattcatcaaaccgattttggagaagatgga |
| | | | tggtaccgaggagctgctggtaaaattgaatcgggaagacctcttgcgaa |
| | | | agcagcgtactttcgataatggtagcatcccgcaccagattcatctgggc |
| | | | gaacttcacgcaatcctacgtcgtcaagaggatttctacccgtttctgaa |
| | | | ggataaccgtgaaaagatcgagaagattctgacctttcgtattccgtatt |
| | | | atgtgggtccgctggcgagaggcaacagccgttttgcgtggatgacccgc |
| | | | aagtcggaagagactatcacgccgtggaatttcgaggaggtcgtggacaa |
| | | | gggtgcatcagcgcaatcttttattgaacgtatgaccaattttgataaaa |
| | | | accttccaaatgaaaaagtgttgccgaagcacagcttgctgtacgagtat |
| | | | ttcaccgtttacaatgaactgacgaaagtcaagtacgttaccgaaggtat |
| | | | gcgtaagccagcctttctgtctggggagcaaaaaaaagccattgtggatc |
| | | | tgctgttcaagaccaatcgcaaggttaccgtcaagcaactgaaagaggat |
| | | | tacttcaaaaaaatcgaatgttttgactccgtggaaatcagcggcgttga |
| | | | agaccgctttaatgcatctctgggcacctaccacgatctactcaagatca |
| | | | tcaaggataaggacttcctggacaacgaagaaaatgaggatatcttggaa |
| | | | gatattgtgctgactttaacgttgttcgaggatcgtgagatgattgaaga |
| | | | acgcctcaaaacgtacgcacatctgtttgacgataaggtgatgaaacagc |
| | | | taaagcgccgtcgttacaccggttggggtcgtctgtctagaaagctgatt |
| | | | aacggcattcgtgacaaacaatctggcaagaccatcctggacttcctcaa |
| | | | atccgacggcttcgcaaatcgtaatttcatgcaactgatccatgatgata |
| | | | gcctgacctttaaagaagatattcagaaggctcaagtgtccggccagggt |
| | | | gactcgctgcatgagcatatcgcgaatctggctggctcccctgcgataaa |
| | | | aaaaggtattctccaaacggtgaaagttgtggatgaactggttaaagtca |
| | | | tgggtcgtcataaaccggaaaacatcgtgattgagatggcacgtgagaac |
| | | | cagaccacccagaaaggtcaaaaaaacagtcgtgagcgtatgaaacgtat |
| | | | cgaagaggggattaaggagttaggcagccagattctgaaggagcacccgg |
| | | | ttgagaacacccagctgcaaaacgagaagctctacctgtattacctgcaa |
| | | | aacggccgtgacatgtatgttgaccaggagctggacatcaatcgtctcag |
| | | | cgactacgacgtggatgctatcgtgccgcagagctttctaaaagatgatt |
| | | | ccatcgacaataaagtgctcacccgtagcgacaagaaccgtggtaaaagc |
| | | | gacaacgtgccgagcgaggaagtagttaagaaaatgaaaaactactggcg |
| | | | tcagctattgaacgccaagcttatcacccagcgtaagtttgacaacttga |
| | | | cgaaggcagagcgtggcggcctgagcgagctagacaaggcgggttttatt |
| | | | aaacgccagctggtggaaactcgccagattaccaaacacgttgcgcaaat |
| | | | cttggactcacgcatgaatacgaagtacgacgaaaacgataaattgatcc |
| | | | gcgaggtgaaggtcataaccctgaaatctaaactggttagcgatttccgg |
| | | | aaggactttcaattttataaagtccgggagattaacaactatcaccatgc |
| | | | acacgacgcttacttaaatgcggttgttggcacggccctgatcaagaagt |
| | | | acccgaaactggaaagcgagtttgtttatggtgactacaaggtgtatgac |
| | | | gttcgcaagatgattgcaaaatccgaacaagagattggtaaggcgacggc |
| | | | taagtattttttctacagcaatatcatgaacttcttcaagactgaaatca |
| | | | ccctggcgaacggtgaaattcgtaaacgtccgttgatcgaaaccaacggt |
| | | | gaaaccggtgaaatcgtttgggataaaggtagggattttgcgaccgttcg |
| | | | taaggtcctgagcatgccgcaggttaacatcgtgaaaaagaccgaagtgc |
| | | | agaccggcggtttctccaaggagagtatcttaccgaagcgcaacagtgac |
| | | | aagctgatcgcgcgtaaaaaagactgggatccgaaaaaatacggcggctt |
| | | | tgactccccgaccgtcgcgtacagcgtactggtcgtggctaaagtggaga |
| | | | aaggcaaatcgaaaaagctgaaatccgttaaagagttgctgggtattacg |
| | | | attatggaacgttcgagcttcgagaaaaatccgatcgacttcctggaggc |
| | | | caaaggctacaaggaagttaaaaaagacctgatcattaaactgccgaagt |
| | | | acagcctgttcgagctcgagaacggtcgcaaacgcatgctggcgagcgcg |
| | | | ggtgagctccaaaaaggcaacgaattggctctgccgagcaagtacgtgaa |
| | | | cttcctgtatctggcctcccattatgaaaaactgaagggtagcccggaag |
| | | | ataacgaacaaaagcagctgttcgtggaacaacataaacactatctggac |
| | | | gagattattgagcaaatcagcgagttcagcaaacgcgtgattctggcgga |
| | | | tgctaatctggacaaggttctttcagcctataataaacaccgtgataagc |
| | | | cgatccgtgaacaggcagaaaatattattcacctgttcaccctgacgaac |
| | | | ctgggtgcgccagcagcgtttaaatacttcgacacaacgatcgaccggaa |
| | | | gcgttataccagcaccaaagaggttcttgacgccaccttgattcaccaaa |
| | | | gcatcacgggtctgtatgaaacccgtattgacttgagccagttgggcgga |
| | | | gattcagggggaagtggcggaagcggtgggtctacgaacctgagcgacat |
| | | | tattgaaaaagaaactggtaaacagttggtgatccaagaatccattctga |
| | | | tgttaccggaggaagtggaagaggtgatcggcaacaagccggagtccgac |
| | | | atcctggttcacaccgcgtatgatgagtcgaccgatgagaatgttatgct |
| | | | gctgaccagcgacgccccagagtacaaaccgtgggcgctcgtcatccagg |
| | | | atagcaatggcgaaaacaagatcaagatgttgtctggcggtagcatggct |
| | | | ccgaaaaagaagcgtaaagtaggtattcatggtgttccggcatcaggtta |
| | | | a |
| 26 | Base editor | evoCDA1- | atgggaagtcatcaccaccaccaccattcaggtgagaacctgtacttcca |
| | | dCasPhi-2 | gagcatggctccgaaaaagaaacgtaaggtgggtatccacggcgttccgg |
| | | | cgatgtcaacagatgctgaatatgtaaggatacacgagaagctggacatc |
| | | | tacacctttaaaaagcagttctccaacaataaaaaatctgtcagccatcg |
| | | | ttgctacgtgttgtttgaactcaagcgtcgtggcgaacgccgtgcgtgct |
| | | | tttggggttacgctgtgaacaaaccgcagagcggtacggaacgtggtatt |
| | | | catgcggagatcttcagcatccgcaaggtggaagagtatctgcgcgataa |
| | | | cccgggtcaattcaccattaactggtacagcagctggtcaccgtgcgccg |
| | | | actgtgctgagaagattttggagtggtataaccaagaattgcgcggtaat |
| | | | ggccatactctgaagatctgggtgtgtaaactttattacgaaaagaacgc |
| | | | gcgtaatcaaattggtctgtggaatctgagagataacggcgttggtctga |
| | | | atgttatggtttccgagcactatcagtgctgccgtaaaatcttcattcag |
| | | | tccagccacaaccaactgaacgagaaccgttggctggaaaagaccctgaa |
| | | | gcgcgcggagaaacgtcgtagcgagctgagcattatgtttcaggttaaaa |
| | | | tcttacacaccaccaaatcgccggcagtctctggcggttcctcgggcggc |
| | | | agctctggctccgaaaccccaggtacgagcgagagcgcaaccccggaatc |
| | | | tggaagtgagacacccgggacttcagaatccgcgaccccggagtctggaa |
| | | | tgccaaaacccgctgtagagtcagaattttcaaaagttttgaaaaagcac |
| | | | tttccgggagaacgtttccgctcgagctatatgaaaagaggcggtaagat |
| | | | cctggctgcccaaggcgaagaggcagttgttgcgtatctacagggcaagt |
| | | | ccgaggaggagcctccgaacttccagccaccggcgaagtgccacgtggtg |
| | | | accaaatcccgtgacttcgcggaatggccgatcatgaaggcgtcggaagc |
| | | | gattcaacgttatatctatgcactgtcgaccaccgaacgcgcggcctgta |
| | | | aaccgggcaaaagtagtgagagccatgcagcgtggttcgccgctaccggt |
| | | | gtcagcaaccacggctacagccacgttcagggtctgaacctgatttttga |
| | | | tcataccttgggccgttacgatggtgttctgaaaaaggtccaactgcgca |
| | | | acgaaaaggcgagagcgcgtttagagagcattaacgcgtctagagcagat |
| | | | gaaggtttaccggagattaaagccgaggaggaagaagtggctacgaacga |
| | | | aaccggccacctgttgcaaccgccgggtatcaatccgagcttttacgtgt |
| | | | atcagaccattagcccgcaggcataccgtccgcgtgacgagatcgtgctg |
| | | | ccgccggagtacgcaggttacgtacgtgacccgaatgctccgattccgct |
| | | | gggtgtggtgcgtaaccgttgcgatatccaaaaagggtgcccgggctaca |
| | | | tcccggaatggcagcgtgaagcaggtaccgcgatcagcccgaaaacaggc |
| | | | aaggccgtaactgtgccgggcctgtctccgaagaaaaacaaacgtatgcg |
| | | | taggtactggcgctctgagaaggagaaagctcaagatgcgctgttggtta |
| | | | ccgtacgcattggtaccgattgggttgtcatcgacgtgcgcggtctgttg |
| | | | cgcaacgcccgttggcgcaccatcgctcccaaggacatcagcctgaatgc |
| | | | gctcctcgatttgtttaccggtgatccggtgatcgatgtgcgtcgcaata |
| | | | ttgttacctttacgtacaccctcgacgcctgtggtacctacgcccgtaaa |
| | | | tggaccttgaaaggcaaacagacgaaagcgaccctggacaaactgacggc |
| | | | cactcagacggttgcgttggttgcgattgcgcttggtcagaccaacccga |
| | | | tttccgctggcattagccgtgttacacaagaaaatggtgcactgcaatgc |
| | | | gaaccgctggaccggttcaccctgccggatgacctgttgaaggacatcag |
| | | | cgcatatcgcattgcttgggacagaaacgaagaggaactacgcgctcgca |
| | | | gcgtcgaggccctgccggaggcgcaacaagcggaggtccgtgcgctggac |
| | | | ggggtgtccaaagaaaccgcgcgtacccaactgtgcgcagatttcggcct |
| | | | ggacccgaaacgtctgccgtgggataaaatgagctccaatactaccttta |
| | | | tcagcgaagccttgctgtctaacagcgtgagccgtgatcaggtttttttc |
| | | | actccggctccgaagaagggtgctaagaaaaaagcgcctgttgaagttat |
| | | | gcgtaaagaccgcacttgggcacgtgcgtacaagccgcgtctgagcgtcg |
| | | | aagctcagaaactgaagaacgaagccctgtgggcacttaagcgcacctcc |
| | | | ccggagtatttgaagctgagccgtcgcaaagaggagctgtgtcgtcgtag |
| | | | catcaactatgttatcgaaaagacccgtcgccgtacgcaatgtcagattg |
| | | | tgatcccggtgatcgaggatctgaatgttcgcttctttcatggttctggt |
| | | | aagagactgcccggttgggataatttcttcaccgctaaaaaagagaaccg |
| | | | ttggttcatccagggcctgcacaaagcgtttagcgatctgcgtacgcacc |
| | | | gttccttctatgtttttgaagttcgtccggagcgcacctctattacctgt |
| | | | ccaaagtgcggtcattgcgaggtgggtaatcgtgacggtgaggcgtttca |
| | | | gtgcctgagctgcggcaagacgtgcaacgcagatcttgacgtcgcgaccc |
| | | | ataatctgacccaagtggcgttgaccggtaagaccatgccgaagcgcgag |
| | | | gagccgcgtgacgctcagggtacagcgccagcgcgcaagactaagaaggc |
| | | | gagcaaatcaaaggcgccaccggcggagcgcgaagaccagaccccggcgc |
| | | | aagagccaagccagaccagttctggaggtagcgggggtagcggtggtagc |
| | | | actaacttaagtgatattatcgagaaagaaaccgggaaacagctggtgat |
| | | | ccaagaaagcattctgatgctgcccgaagaagttgaagaagtgattggta |
| | | | acaagcctgagtcggacatcttagtgcacaccgcgtacgatgaaagcacg |
| | | | gatgagaatgtgatgctgctcacctctgatgcccccgaatacaagccgtg |
| | | | ggctctggtgatccaggactctaacggagaaaacaaaattaagatgctct |
| | | | ctggcggctccatggcgcctaaaaaaaaacgcaaagtggggattcacggg |
| | | | gttccggcgtaa |
| 27 | Base editor | evoCDA1- | atgggaagtcatcaccaccaccaccattcaggtgagaacctgtacttcca |
| | | dLbCas12a | gagcatggctccgaaaaagaaacgtaaggtgggtatccacggcgttccgg |
| | | | cgatgtcaacagatgctgaatatgtaaggatacacgagaagctggacatc |
| | | | tacacctttaaaaagcagttctccaacaataaaaaatctgtcagccatcg |
| | | | ttgctacgtgttgtttgaactcaagcgtcgtggcgaacgccgtgcgtgct |
| | | | tttggggttacgctgtgaacaaaccgcagagcggtacggaacgtggtatt |
| | | | catgcggagatcttcagcatccgcaaggtggaagagtatctgcgcgataa |
| | | | cccgggtcaattcaccattaactggtacagcagctggtcaccgtgcgccg |
| | | | actgtgctgagaagattttggagtggtataaccaagaattgcgcggtaat |
| | | | ggccatactctgaagatctgggtgtgtaaactttattacgaaaagaacgc |
| | | | gcgtaatcaaattggtctgtggaatctgagagataacggcgttggtctga |
| | | | atgttatggtttccgagcactatcagtgctgccgtaaaatcttcattcag |
| | | | tccagccacaaccaactgaacgagaaccgttggctggaaaagaccctgaa |
| | | | gcgcgcggagaaacgtcgtagcgagctgagcattatgtttcaggttaaaa |
| | | | tcttacacaccaccaaatcgccggcagtctctggcggttcctcgggcggc |
| | | | agctctggctccgaaaccccaggtacgagcgagagcgcaaccccggaatc |
| | | | tggaagtgagacacccgggacttcagaatccgcgaccccggagtctggaa |
| | | | tgtcaaaactagaaaagtttacaaattgttactccctgtcgaaaaccctg |
| | | | agattcaaagcgatcccggtgggtaaaactcaagagaacattgataacaa |
| | | | acgtctgctggttgaggacgaaaagcgcgcggaggattacaaaggcgtta |
| | | | aaaagctgttggaccgttattacctgtcctttattaatgatgttctgcac |
| | | | agcattaaattaaagaacctaaacaattacatctccctcttccgtaaaaa |
| | | | gacccgcaccgaaaaggaaaataaagaactggagaaccttgagattaatc |
| | | | tgcgtaaagaaatcgctaaagcgtttaaaggcaacgagggttataaatcc |
| | | | ttatttaaaaaagacatcatcgaaacgattctcccagaattcctggacga |
| | | | caaagacgagatcgcgctggttaattccttcaacggctttaccaccgcat |
| | | | tcaccggcttttttgataaccgcgaaaacatgttctctgaagaggccaaa |
| | | | agcacctcaattgcgtttcgctgcattaacgaaaatctgacgcgttatat |
| | | | ctctaacatggatatcttcgagaaggttgacgctatctttgataaacatg |
| | | | aagtccaggagatcaaggagaagatcctgaacagcgactacgacgtggag |
| | | | gatttcttcgagggtgagttcttcaattttgttttgacccaagaaggcat |
| | | | cgatgtctacaatgcgataattggtggttttgttaccgagagcggggaga |
| | | | aaattaagggtctgaacgaatatatcaacttgtacaaccagaaaaccaaa |
| | | | caaaaactgccgaagtttaagccgctgtacaagcaagtcttgtcggatcg |
| | | | tgaatcgctgagcttttacggcgaaggctacaccagcgatgaggaggtgt |
| | | | tggaggtgttccgcaatacgttgaataagaactctgaaattttttccagc |
| | | | attaagaagctggaaaaactattcaagaacttcgacgagtatagcagcgc |
| | | | aggtatctttgtgaaaaacggtccggctattagcaccatctcgaaagaca |
| | | | ttttcggcgaatggaatgttattcgtgataagtggaatgccgaatacgac |
| | | | gacattcatctgaagaaaaaggcggttgtaactgaaaagtacgaggacga |
| | | | tcgtcgtaagtcgttcaaaaagatcggctccttctctctggagcaactgc |
| | | | aagagtacgccgatgccgatttgtcagtcgtggagaagctgaaagagatc |
| | | | atcattcaaaaagttgacgaaatctataaggtctatggtagcagtgaaaa |
| | | | actgtttgacgcggacttcgtgctggagaaaagcctgaagaaaaatgacg |
| | | | ccgtggtggcaatcatgaaagatcttctggatagcgtcaagtctttcgaa |
| | | | aactacatcaaagcgtttttcggtgaaggcaaggaaacgaatcgtgacga |
| | | | gagcttctatggtgatttcgtcctagcctatgatatcctgcttaaggtgg |
| | | | atcacatctatgatgccatccgcaactatgttacccagaaaccgtactca |
| | | | aaggacaagttcaagttatacttccagaatccgcagtttatgggtggctg |
| | | | ggataaagataaggaaaccgactatcgcgccaccattttacgttacggta |
| | | | gtaagtactatctggcaatcatggataaaaaatatgcgaaatgcctgcaa |
| | | | aagatcgacaaggacgatgttaacggcaattatgagaagatcaactacaa |
| | | | actgctgccgggcccaaataagatgctgccgaaggtgtttttcagcaaaa |
| | | | aatggatggcgtattacaatccgagcgaggacatccaaaagatctacaaa |
| | | | aacggtacgtttaagaagggtgatatgttcaacctcaacgattgtcataa |
| | | | acttattgacttcttcaaggatagcatatctcgttatccgaagtggagca |
| | | | acgcgtatgactttaactttagcgaaaccgagaaatacaaagatatcgcg |
| | | | ggtttctatcgtgaagttgaagaacagggttacaaagtgtccttcgagtc |
| | | | cgcgagcaagaaggaggtagacaagctggtcgaggagggtaagttgtaca |
| | | | tgtttcagatttacaataaggacttttccgacaagtcccatggtacaccg |
| | | | aatttgcacaccatgtatttcaagcttctgttcgacgagaacaaccacgg |
| | | | ccagatccgtctgagcggtggtgcagagctgttcatgcgtagagctagcc |
| | | | tgaagaaagaagagttggtcgttcatccggctaattcgccgatcgcgaac |
| | | | aaaaatccagataaccctaaaaagactacgaccctgagctatgatgtgta |
| | | | taaagacaaacgctttagcgaagaccagtatgagttacacattccgatcg |
| | | | ctatcaacaagtgcccgaagaatattttcaagatcaacaccgaagtgcgt |
| | | | gttcttttgaagcacgatgacaacccgtatgtgattggtattgctcgtgg |
| | | | tgaacgtaatctgctgtacattgtggttgtggacggcaaaggaaacatcg |
| | | | tggaacagtacagcctgaacgaaatcattaacaatttcaacggcattcgc |
| | | | attaagacggattaccattctttgctggacaaaaaggagaaagagcgttt |
| | | | cgaggcccgtcagaactggacctccatcgagaacattaaggagctgaagg |
| | | | cgggttacatcagccaggtggtgcacaaaatctgcgaactggtggagaag |
| | | | tacgacgcggtgattgctttggcggatttgaactccggttttaagaacag |
| | | | ccgcgttaaagtggaaaagcaggtttatcagaaattcgaaaaaatgctga |
| | | | ttgataaattgaattacatggttgacaaaaagtctaacccgtgtgcaacc |
| | | | ggcggtgcgctgaagggctaccaaatcaccaataagtttgaaagctttaa |
| | | | gagcatgagcacgcaaaatggcttcattttttacattccggcgtggctga |
| | | | ccagcaagatcgatccgtcaaccggctttgttaatttactgaagacgaag |
| | | | tatacctctattgccgactctaaaaaatttattagttccttcgatagaat |
| | | | catgtatgttccggaagaggacctgttcgaatttgcactggactacaaga |
| | | | actttagccgtaccgatgctgattatataaagaaatggaaactctacagc |
| | | | tatggcaaccgcatccgtatctttcgcaacccgaaaaaaaacaacgtgtt |
| | | | cgactgggaagaggtatgtctgacttcagcgtacaaggagcttttcaata |
| | | | aatatggtatcaactaccagcagggtgatatccgtgcactgctttgcgag |
| | | | caaagcgataaagcgttctattcaagcttcatggcgctgatgagcctgat |
| | | | gctgcaaatgcgcaacagcatcaccggtcgtaccgatgttgcatttctga |
| | | | tttctccagtaaaaaacagcgatggtatcttttacgacagccgtaattac |
| | | | gaggcccaagaaaatgcgattttgccgaagaacgctgacgcgaatggtgc |
| | | | atacaacattgcaaggaaagttctgtgggcaatcggccaatttaaaaaag |
| | | | cggaagatgagaagctggacaaagtgaaaattgcgattagcaacaaggag |
| | | | tggctggagtatgcgcagacctccgttaaatcagggggaagtggcggaag |
| | | | cggtgggtctacgaacctgagcgacattattgaaaaagaaactggtaaac |
| | | | agttggtgatccaagaatccattctgatgttaccggaggaagtggaagag |
| | | | gtgatcggcaacaagccggagtccgacatcctggttcacaccgcgtatga |
| | | | tgagtcgaccgatgagaatgttatgctgctgaccagcgacgccccagagt |
| | | | acaaaccgtgggcgctcgtcatccaggatagcaatggcgaaaacaagatc |
| | | | aagatgttgtctggcggtagcatggctccgaaaaagaagcgtaaagtagg |
| | | | tattcatggtgttccggcatcaggttaa |
| 28 | Base editor | evoFERNY-dCas9 | atgggaagtcatcaccaccaccaccattcaggtgagaacctgtacttcca |
| | | | gagcatggctccgaaaaagaaacgtaaggtgggtatccacggcgttccgg |
| | | | cgatgtcatttgagaggaattatgatcccagagaattgcgcaaagagacg |
| | | | tacctgctgtatgaaatcaagtggggtaaaagcggcaagctgtggcgtca |
| | | | ttggtgtcaaaacaaccgcacccaacacgcggaagtttatttcctcgaga |
| | | | atattttcaacgcccgtcgttttaacccgagcacccactgctccatcacc |
| | | | tggtatctttcttggtcgccgtgtgcggagtgctcgcaaaaaatcgtgga |
| | | | cttcctgaaagaacatccgaatgtgaacctggagatctatgttgcgagat |
| | | | tgtactatccggagaacgaacgtaatcgtcagggcctgcgcgacttggtc |
| | | | aacagcggggtgaccattcgtattatggatctgccggattacaattactg |
| | | | ctggaaaacctttgtttccgatcagggtggcgacgaagactactggcctg |
| | | | gtcactttgctccgtggattaagcagtacagcctgaagctgagcggtggt |
| | | | tcttccggcggtagcagtggctccgagactccaggtacgagcgagagcgc |
| | | | aaccccggaatctggaagtgagacacccgggacttcagaatccgcgaccc |
| | | | cggagtctggaatggataaaaagtattcaataggactagctattggcacc |
| | | | aacagcgttggttgggcagttatcaccgatgaatataaggtcccgagcaa |
| | | | gaagtttaaggtcctgggcaacaccgatcgccacagcataaaaaagaacc |
| | | | ttattggtgcgctgctgttcgattctggtgagactgccgaagcgacccgt |
| | | | ctgaagagaaccgcgcgtcgtagatacacccggcgcaagaaccgtatctg |
| | | | ctatctgcaagaaattttcagcaacgagatggctaaggtcgacgactcct |
| | | | tctttcatcgcctggaggaaagctttctggttgaggaagacaaaaagcac |
| | | | gagcgtcatccgattttcggtaacattgttgatgaagttgcctaccacga |
| | | | aaaataccccacaatttatcatctgcgtaaaaaactggtagactccaccg |
| | | | ataaggcggatttacgccttatctatttggcgctggcgcatatgattaag |
| | | | ttccgcggtcattttttgatcgagggcgacctgaatccggataacagcga |
| | | | cgttgacaaactgtttatccagctggtgcagacctataaccagttgttcg |
| | | | aagagaacccgattaatgcgtctggcgtggacgcgaaggcgatcctgagc |
| | | | gctcgtctgtccaagtcacgccgcctggagaacttgatcgcgcaactgcc |
| | | | gggtgagaagaaaaacggcttgttcggcaatctgattgctctgtccctag |
| | | | gtttgaccccgaatttcaaatctaattttgatctggcggaagatgcgaaa |
| | | | ttacagttgagcaaggacacttacgatgacgatctggacaacctgctcgc |
| | | | tcagatcggcgatcagtatgccgatcttttcctggccgcgaagaacctga |
| | | | gcgatgccatcttgctatcggacatcttacgtgttaacaccgaaatcacc |
| | | | aaagctccgctaagcgcgtccatgattaaacgttacgatgaacaccacca |
| | | | ggatctgacgctgctgaaagcgttggtgcgccagcaactgccggaaaaat |
| | | | acaaggaaattttcttcgaccaaagcaagaacggttatgcaggctatatc |
| | | | gacggtggtgcgtcgcaagaagaattttataaattcatcaaaccgatttt |
| | | | ggagaagatggatggtaccgaggagctgctggtaaaattgaatcgggaag |
| | | | acctcttgcgaaagcagcgtactttcgataatggtagcatcccgcaccag |
| | | | attcatctgggcgaacttcacgcaatcctacgtcgtcaagaggatttcta |
| | | | cccgtttctgaaggataaccgtgaaaagatcgagaagattctgacctttc |
| | | | gtattccgtattatgtgggtccgctggcgagaggcaacagccgttttgcg |
| | | | tggatgacccgcaagtcggaagagactatcacgccgtggaatttcgagga |
| | | | ggtcgtggacaagggtgcatcagcgcaatcttttattgaacgtatgacca |
| | | | attttgataaaaaccttccaaatgaaaaagtgttgccgaagcacagcttg |
| | | | ctgtacgagtatttcaccgtttacaatgaactgacgaaagtcaagtacgt |
| | | | taccgaaggtatgcgtaagccagcctttctgtctggggagcaaaaaaaag |
| | | | ccattgtggatctgctgttcaagaccaatcgcaaggttaccgtcaagcaa |
| | | | ctgaaagaggattacttcaaaaaaatcgaatgttttgactccgtggaaat |
| | | | cagcggcgttgaagaccgctttaatgcatctctgggcacctaccacgatc |
| | | | tactcaagatcatcaaggataaggacttcctggacaacgaagaaaatgag |
| | | | gatatcttggaagatattgtgctgactttaacgttgttcgaggatcgtga |
| | | | gatgattgaagaacgcctcaaaacgtacgcacatctgtttgacgataagg |
| | | | tgatgaaacagctaaagcgccgtcgttacaccggttggggtcgtctgtct |
| | | | agaaagctgattaacggcattcgtgacaaacaatctggcaagaccatcct |
| | | | ggacttcctcaaatccgacggcttcgcaaatcgtaatttcatgcaactga |
| | | | tccatgatgatagcctgacctttaaagaagatattcagaaggctcaagtg |
| | | | tccggccagggtgactcgctgcatgagcatatcgcgaatctggctggctc |
| | | | ccctgcgataaaaaaaggtattctccaaacggtgaaagttgtggatgaac |
| | | | tggttaaagtcatgggtcgtcataaaccggaaaacatcgtgattgagatg |
| | | | gcacgtgagaaccagaccacccagaaaggtcaaaaaaacagtcgtgagcg |
| | | | tatgaaacgtatcgaagaggggattaaggagttaggcagccagattctga |
| | | | aggagcacccggttgagaacacccagctgcaaaacgagaagctctacctg |
| | | | tattacctgcaaaacggccgtgacatgtatgttgaccaggagctggacat |
| | | | caatcgtctcagcgactacgacgtggatgctatcgtgccgcagagctttc |
| | | | taaaagatgattccatcgacaataaagtgctcacccgtagcgacaagaac |
| | | | cgtggtaaaagcgacaacgtgccgagcgaggaagtagttaagaaaatgaa |
| | | | aaactactggcgtcagctattgaacgccaagcttatcacccagcgtaagt |
| | | | ttgacaacttgacgaaggcagagcgtggcggcctgagcgagctagacaag |
| | | | gcgggttttattaaacgccagctggtggaaactcgccagattaccaaaca |
| | | | cgttgcgcaaatcttggactcacgcatgaatacgaagtacgacgaaaacg |
| | | | ataaattgatccgcgaggtgaaggtcataaccctgaaatctaaactggtt |
| | | | agcgatttccggaaggactttcaattttataaagtccgggagattaacaa |
| | | | ctatcaccatgcacacgacgcttacttaaatgcggttgttggcacggccc |
| | | | tgatcaagaagtacccgaaactggaaagcgagtttgtttatggtgactac |
| | | | aaggtgtatgacgttcgcaagatgattgcaaaatccgaacaagagattgg |
| | | | taaggcgacggctaagtattttttctacagcaatatcatgaacttcttca |
| | | | agactgaaatcaccctggcgaacggtgaaattcgtaaacgtccgttgatc |
| | | | gaaaccaacggtgaaaccggtgaaatcgtttgggataaaggtagggattt |
| | | | tgcgaccgttcgtaaggtcctgagcatgccgcaggttaacatcgtgaaaa |
| | | | agaccgaagtgcagaccggcggtttctccaaggagagtatcttaccgaag |
| | | | cgcaacagtgacaagctgatcgcgcgtaaaaaagactgggatccgaaaaa |
| | | | atacggcggctttgactccccgaccgtcgcgtacagcgtactggtcgtgg |
| | | | ctaaagtggagaaaggcaaatcgaaaaagctgaaatccgttaaagagttg |
| | | | ctgggtattacgattatggaacgttcgagcttcgagaaaaatccgatcga |
| | | | cttcctggaggccaaaggctacaaggaagttaaaaaagacctgatcatta |
| | | | aactgccgaagtacagcctgttcgagctcgagaacggtcgcaaacgcatg |
| | | | ctggcgagcgcgggtgagctccaaaaaggcaacgaattggctctgccgag |
| | | | caagtacgtgaacttcctgtatctggcctcccattatgaaaaactgaagg |
| | | | gtagcccggaagataacgaacaaaagcagctgttcgtggaacaacataaa |
| | | | cactatctggacgagattattgagcaaatcagcgagttcagcaaacgcgt |
| | | | gattctggcggatgctaatctggacaaggttctttcagcctataataaac |
| | | | accgtgataagccgatccgtgaacaggcagaaaatattattcacctgttc |
| | | | accctgacgaacctgggtgcgccagcagcgtttaaatacttcgacacaac |
| | | | gatcgaccggaagcgttataccagcaccaaagaggttcttgacgccacct |
| | | | tgattcaccaaagcatcacgggtctgtatgaaacccgtattgacttgagc |
| | | | cagttgggcggagattcagggggaagtggcggaagcggtgggtctacgaa |
| | | | cctgagcgacattattgaaaaagaaactggtaaacagttggtgatccaag |
| | | | aatccattctgatgttaccggaggaagtggaagaggtgatcggcaacaag |
| | | | ccggagtccgacatcctggttcacaccgcgtatgatgagtcgaccgatga |
| | | | gaatgttatgctgctgaccagcgacgccccagagtacaaaccgtgggcgc |
| | | | tcgtcatccaggatagcaatggcgaaaacaagatcaagatgttgtctggc |
| | | | ggtagcatggctccgaaaaagaagcgtaaagtaggtattcatggtgttcc |
| | | | ggcatcaggttaa |
| 29 | Base editor | evoFERNY- | atgggaagtcatcaccaccaccaccattcaggtgagaacctgtacttcca |
| | | dCasPhi-2 | gagcatggctccgaaaaagaaacgtaaggtgggtatccacggcgttccgg |
| | | | cgatgtcatttgagaggaattatgatcccagagaattgcgcaaagagacg |
| | | | tacctgctgtatgaaatcaagtggggtaaaagcggcaagctgtggcgtca |
| | | | ttggtgtcaaaacaaccgcacccaacacgcggaagtttatttcctcgaga |
| | | | atattttcaacgcccgtcgttttaacccgagcacccactgctccatcacc |
| | | | tggtatctttcttggtcgccgtgtgcggagtgctcgcaaaaaatcgtgga |
| | | | cttcctgaaagaacatccgaatgtgaacctggagatctatgttgcgagat |
| | | | tgtactatccggagaacgaacgtaatcgtcagggcctgcgcgacttggtc |
| | | | aacagcggggtgaccattcgtattatggatctgccggattacaattactg |
| | | | ctggaaaacctttgtttccgatcagggtggcgacgaagactactggcctg |
| | | | gtcactttgctccgtggattaagcagtacagcctgaagctgagcggtggt |
| | | | tcttccggcggtagcagtggctccgagactccaggtacgagcgagagcgc |
| | | | aaccccggaatctggaagtgagacacccgggacttcagaatccgcgaccc |
| | | | cggagtctggaatgccaaaacccgctgtagagtcagaattttcaaaagtt |
| | | | ttgaaaaagcactttccgggagaacgtttccgctcgagctatatgaaaag |
| | | | aggcggtaagatcctggctgcccaaggcgaagaggcagttgttgcgtatc |
| | | | tacagggcaagtccgaggaggagcctccgaacttccagccaccggcgaag |
| | | | tgccacgtggtgaccaaatcccgtgacttcgcggaatggccgatcatgaa |
| | | | ggcgtcggaagcgattcaacgttatatctatgcactgtcgaccaccgaac |
| | | | gcgcggcctgtaaaccgggcaaaagtagtgagagccatgcagcgtggttc |
| | | | gccgctaccggtgtcagcaaccacggctacagccacgttcagggtctgaa |
| | | | cctgatttttgatcataccttgggccgttacgatggtgttctgaaaaagg |
| | | | tccaactgcgcaacgaaaaggcgagagcgcgtttagagagcattaacgcg |
| | | | tctagagcagatgaaggtttaccggagattaaagccgaggaggaagaagt |
| | | | ggctacgaacgaaaccggccacctgttgcaaccgccgggtatcaatccga |
| | | | gcttttacgtgtatcagaccattagcccgcaggcataccgtccgcgtgac |
| | | | gagatcgtgctgccgccggagtacgcaggttacgtacgtgacccgaatgc |
| | | | tccgattccgctgggtgtggtgcgtaaccgttgcgatatccaaaaagggt |
| | | | gcccgggctacatcccggaatggcagcgtgaagcaggtaccgcgatcagc |
| | | | ccgaaaacaggcaaggccgtaactgtgccgggcctgtctccgaagaaaaa |
| | | | caaacgtatgcgtaggtactggcgctctgagaaggagaaagctcaagatg |
| | | | cgctgttggttaccgtacgcattggtaccgattgggttgtcatcgacgtg |
| | | | cgcggtctgttgcgcaacgcccgttggcgcaccatcgctcccaaggacat |
| | | | cagcctgaatgcgctcctcgatttgtttaccggtgatccggtgatcgatg |
| | | | tgcgtcgcaatattgttacctttacgtacaccctcgacgcctgtggtacc |
| | | | tacgcccgtaaatggaccttgaaaggcaaacagacgaaagcgaccctgga |
| | | | caaactgacggccactcagacggttgcgttggttgcgattgcgcttggtc |
| | | | agaccaacccgatttccgctggcattagccgtgttacacaagaaaatggt |
| | | | gcactgcaatgcgaaccgctggaccggttcaccctgccggatgacctgtt |
| | | | gaaggacatcagcgcatatcgcattgcttgggacagaaacgaagaggaac |
| | | | tacgcgctcgcagcgtcgaggccctgccggaggcgcaacaagcggaggtc |
| | | | cgtgcgctggacggggtgtccaaagaaaccgcgcgtacccaactgtgcgc |
| | | | agatttcggcctggacccgaaacgtctgccgtgggataaaatgagctcca |
| | | | atactacctttatcagcgaagccttgctgtctaacagcgtgagccgtgat |
| | | | caggtttttttcactccggctccgaagaagggtgctaagaaaaaagcgcc |
| | | | tgttgaagttatgcgtaaagaccgcacttgggcacgtgcgtacaagccgc |
| | | | gtctgagcgtcgaagctcagaaactgaagaacgaagccctgtgggcactt |
| | | | aagcgcacctccccggagtatttgaagctgagccgtcgcaaagaggagct |
| | | | gtgtcgtcgtagcatcaactatgttatcgaaaagacccgtcgccgtacgc |
| | | | aatgtcagattgtgatcccggtgatcgaggatctgaatgttcgcttcttt |
| | | | catggttctggtaagagactgcccggttgggataatttcttcaccgctaa |
| | | | aaaagagaaccgttggttcatccagggcctgcacaaagcgtttagcgatc |
| | | | tgcgtacgcaccgttccttctatgtttttgaagttcgtccggagcgcacc |
| | | | tctattacctgtccaaagtgcggtcattgcgaggtgggtaatcgtgacgg |
| | | | tgaggcgtttcagtgcctgagctgcggcaagacgtgcaacgcagatcttg |
| | | | acgtcgcgacccataatctgacccaagtggcgttgaccggtaagaccatg |
| | | | ccgaagcgcgaggagccgcgtgacgctcagggtacagcgccagcgcgcaa |
| | | | gactaagaaggcgagcaaatcaaaggcgccaccggcggagcgcgaagacc |
| | | | agaccccggcgcaagagccaagccagaccagttctggaggtagcgggggt |
| | | | agcggtggtagcactaacttaagtgatattatcgagaaagaaaccgggaa |
| | | | acagctggtgatccaagaaagcattctgatgctgcccgaagaagttgaag |
| | | | aagtgattggtaacaagcctgagtcggacatcttagtgcacaccgcgtac |
| | | | gatgaaagcacggatgagaatgtgatgctgctcacctctgatgcccccga |
| | | | atacaagccgtgggctctggtgatccaggactctaacggagaaaacaaaa |
| | | | ttaagatgctctctggcggctccatggcgcctaaaaaaaaacgcaaagtg |
| | | | gggattcacggggttccggcgtaa |
| 30 | Base editor | evoFERNY- | atgggaagtcatcaccaccaccaccattcaggtgagaacctgtacttcca |
| | | dLbCas12a | gagcatggctccgaaaaagaaacgtaaggtgggtatccacggcgttccgg |
| | | | cgatgtcatttgagaggaattatgatcccagagaattgcgcaaagagacg |
| | | | tacctgctgtatgaaatcaagtggggtaaaagcggcaagctgtggcgtca |
| | | | ttggtgtcaaaacaaccgcacccaacacgcggaagtttatttcctcgaga |
| | | | atattttcaacgcccgtcgttttaacccgagcacccactgctccatcacc |
| | | | tggtatctttcttggtcgccgtgtgcggagtgctcgcaaaaaatcgtgga |
| | | | cttcctgaaagaacatccgaatgtgaacctggagatctatgttgcgagat |
| | | | tgtactatccggagaacgaacgtaatcgtcagggcctgcgcgacttggtc |
| | | | aacagcggggtgaccattcgtattatggatctgccggattacaattactg |
| | | | ctggaaaacctttgtttccgatcagggtggcgacgaagactactggcctg |
| | | | gtcactttgctccgtggattaagcagtacagcctgaagctgagcggtggt |
| | | | tcttccggcggtagcagtggctccgagactccaggtacgagcgagagcgc |
| | | | aaccccggaatctggaagtgagacacccgggacttcagaatccgcgaccc |
| | | | cggagtctggaatgtcaaaactagaaaagtttacaaattgttactccctg |
| | | | tcgaaaaccctgagattcaaagcgatcccggtgggtaaaactcaagagaa |
| | | | cattgataacaaacgtctgctggttgaggacgaaaagcgcgcggaggatt |
| | | | acaaaggcgttaaaaagctgttggaccgttattacctgtcctttattaat |
| | | | gatgttctgcacagcattaaattaaagaacctaaacaattacatctccct |
| | | | cttccgtaaaaagacccgcaccgaaaaggaaaataaagaactggagaacc |
| | | | ttgagattaatctgcgtaaagaaatcgctaaagcgtttaaaggcaacgag |
| | | | ggttataaatccttatttaaaaaagacatcatcgaaacgattctcccaga |
| | | | attcctggacgacaaagacgagatcgcgctggttaattccttcaacggct |
| | | | ttaccaccgcattcaccggcttttttgataaccgcgaaaacatgttctct |
| | | | gaagaggccaaaagcacctcaattgcgtttcgctgcattaacgaaaatct |
| | | | gacgcgttatatctctaacatggatatcttcgagaaggttgacgctatct |
| | | | ttgataaacatgaagtccaggagatcaaggagaagatcctgaacagcgac |
| | | | tacgacgtggaggatttcttcgagggtgagttcttcaattttgttttgac |
| | | | ccaagaaggcatcgatgtctacaatgcgataattggtggttttgttaccg |
| | | | agagcggggagaaaattaagggtctgaacgaatatatcaacttgtacaac |
| | | | cagaaaaccaaacaaaaactgccgaagtttaagccgctgtacaagcaagt |
| | | | cttgtcggatcgtgaatcgctgagcttttacggcgaaggctacaccagcg |
| | | | atgaggaggtgttggaggtgttccgcaatacgttgaataagaactctgaa |
| | | | attttttccagcattaagaagctggaaaaactattcaagaacttcgacga |
| | | | gtatagcagcgcaggtatctttgtgaaaaacggtccggctattagcacca |
| | | | tctcgaaagacattttcggcgaatggaatgttattcgtgataagtggaat |
| | | | gccgaatacgacgacattcatctgaagaaaaaggcggttgtaactgaaaa |
| | | | gtacgaggacgatcgtcgtaagtcgttcaaaaagatcggctccttctctc |
| | | | tggagcaactgcaagagtacgccgatgccgatttgtcagtcgtggagaag |
| | | | ctgaaagagatcatcattcaaaaagttgacgaaatctataaggtctatgg |
| | | | tagcagtgaaaaactgtttgacgcggacttcgtgctggagaaaagcctga |
| | | | agaaaaatgacgccgtggtggcaatcatgaaagatcttctggatagcgtc |
| | | | aagtctttcgaaaactacatcaaagcgtttttcggtgaaggcaaggaaac |
| | | | gaatcgtgacgagagcttctatggtgatttcgtcctagcctatgatatcc |
| | | | tgcttaaggtggatcacatctatgatgccatccgcaactatgttacccag |
| | | | aaaccgtactcaaaggacaagttcaagttatacttccagaatccgcagtt |
| | | | tatgggtggctgggataaagataaggaaaccgactatcgcgccaccattt |
| | | | tacgttacggtagtaagtactatctggcaatcatggataaaaaatatgcg |
| | | | aaatgcctgcaaaagatcgacaaggacgatgttaacggcaattatgagaa |
| | | | gatcaactacaaactgctgccgggcccaaataagatgctgccgaaggtgt |
| | | | ttttcagcaaaaaatggatggcgtattacaatccgagcgaggacatccaa |
| | | | aagatctacaaaaacggtacgtttaagaagggtgatatgttcaacctcaa |
| | | | cgattgtcataaacttattgacttcttcaaggatagcatatctcgttatc |
| | | | cgaagtggagcaacgcgtatgactttaactttagcgaaaccgagaaatac |
| | | | aaagatatcgcgggtttctatcgtgaagttgaagaacagggttacaaagt |
| | | | gtccttcgagtccgcgagcaagaaggaggtagacaagctggtcgaggagg |
| | | | gtaagttgtacatgtttcagatttacaataaggacttttccgacaagtcc |
| | | | catggtacaccgaatttgcacaccatgtatttcaagcttctgttcgacga |
| | | | gaacaaccacggccagatccgtctgagcggtggtgcagagctgttcatgc |
| | | | gtagagctagcctgaagaaagaagagttggtcgttcatccggctaattcg |
| | | | ccgatcgcgaacaaaaatccagataaccctaaaaagactacgaccctgag |
| | | | ctatgatgtgtataaagacaaacgctttagcgaagaccagtatgagttac |
| | | | acattccgatcgctatcaacaagtgcccgaagaatattttcaagatcaac |
| | | | accgaagtgcgtgttcttttgaagcacgatgacaacccgtatgtgattgg |
| | | | tattgctcgtggtgaacgtaatctgctgtacattgtggttgtggacggca |
| | | | aaggaaacatcgtggaacagtacagcctgaacgaaatcattaacaatttc |
| | | | aacggcattcgcattaagacggattaccattctttgctggacaaaaagga |
| | | | gaaagagcgtttcgaggcccgtcagaactggacctccatcgagaacatta |
| | | | aggagctgaaggcgggttacatcagccaggtggtgcacaaaatctgcgaa |
| | | | ctggtggagaagtacgacgcggtgattgctttggcggatttgaactccgg |
| | | | ttttaagaacagccgcgttaaagtggaaaagcaggtttatcagaaattcg |
| | | | aaaaaatgctgattgataaattgaattacatggttgacaaaaagtctaac |
| | | | ccgtgtgcaaccggcggtgcgctgaagggctaccaaatcaccaataagtt |
| | | | tgaaagctttaagagcatgagcacgcaaaatggcttcattttttacattc |
| | | | cggcgtggctgaccagcaagatcgatccgtcaaccggctttgttaattta |
| | | | ctgaagacgaagtatacctctattgccgactctaaaaaatttattagttc |
| | | | cttcgatagaatcatgtatgttccggaagaggacctgttcgaatttgcac |
| | | | tggactacaagaactttagccgtaccgatgctgattatataaagaaatgg |
| | | | aaactctacagctatggcaaccgcatccgtatctttcgcaacccgaaaaa |
| | | | aaacaacgtgttcgactgggaagaggtatgtctgacttcagcgtacaagg |
| | | | agcttttcaataaatatggtatcaactaccagcagggtgatatccgtgca |
| | | | ctgctttgcgagcaaagcgataaagcgttctattcaagcttcatggcgct |
| | | | gatgagcctgatgctgcaaatgcgcaacagcatcaccggtcgtaccgatg |
| | | | ttgcatttctgatttctccagtaaaaaacagcgatggtatcttttacgac |
| | | | agccgtaattacgaggcccaagaaaatgcgattttgccgaagaacgctga |
| | | | cgcgaatggtgcatacaacattgcaaggaaagttctgtgggcaatcggcc |
| | | | aatttaaaaaagcggaagatgagaagctggacaaagtgaaaattgcgatt |
| | | | agcaacaaggagtggctggagtatgcgcagacctccgttaaatcaggggg |
| | | | aagtggcggaagcggtgggtctacgaacctgagcgacattattgaaaaag |
| | | | aaactggtaaacagttggtgatccaagaatccattctgatgttaccggag |
| | | | gaagtggaagaggtgatcggcaacaagccggagtccgacatcctggttca |
| | | | caccgcgtatgatgagtcgaccgatgagaatgttatgctgctgaccagcg |
| | | | acgccccagagtacaaaccgtgggcgctcgtcatccaggatagcaatggc |
| | | | gaaaacaagatcaagatgttgtctggcggtagcatggctccgaaaaagaa |
| | | | gcgtaaagtaggtattcatggtgttccggcatcaggttaa |
| 31 | Base editor | hA3G-CTD-dCas9 | atgggaagtcatcaccaccaccaccattcaggtgagaacctgtacttcca |
| | | | gagcatggctccgaaaaagaaacgtaaggtgggtatccacggcgttccgg |
| | | | cgatggatcccccaacatttactttcaactttaataacgaaccgtgggtt |
| | | | cgcggtcgccacgaaacctacctgtgctatgaggtggaacgtatgcataa |
| | | | tgatacttgggttctgctgaatcaacgtagaggctttttatgcaaccaag |
| | | | ctccgcataaacatggttttctggaaggccgtcacgccgagctgtgcttc |
| | | | cttgacgtgatcccattctggaaactggacttggaccaggattatcgtgt |
| | | | gacctgtttcaccagctggtcgccgtgcttcagctgtgcgcaggagatgg |
| | | | cgaagttcatctccaaaaacaagcacgttagcttgtgcatttttaccgcg |
| | | | cgtatttacgatgatcaaggtcgctgccaagagggcctgcgtaccctggc |
| | | | agaggctggtgcgaagatctctatcatgacgtacagcgaatttaaacact |
| | | | gctgggacacgtttgtcgatcaccagggttgtccgttccagccgtgggac |
| | | | ggcttggacgaacatagccaggatttgtccggtcgtctgcgcgcaattct |
| | | | acagaatcaagagaactctggaagtgagacacccgggacttcagaatccg |
| | | | cgaccccggagtctggaatggataaaaagtattcaataggactagctatt |
| | | | ggcaccaacagcgttggttgggcagttatcaccgatgaatataaggtccc |
| | | | gagcaagaagtttaaggtcctgggcaacaccgatcgccacagcataaaaa |
| | | | agaaccttattggtgcgctgctgttcgattctggtgagactgccgaagcg |
| | | | acccgtctgaagagaaccgcgcgtcgtagatacacccggcgcaagaaccg |
| | | | tatctgctatctgcaagaaattttcagcaacgagatggctaaggtcgacg |
| | | | actccttctttcatcgcctggaggaaagctttctggttgaggaagacaaa |
| | | | aagcacgagcgtcatccgattttcggtaacattgttgatgaagttgccta |
| | | | ccacgaaaaataccccacaatttatcatctgcgtaaaaaactggtagact |
| | | | ccaccgataaggcggatttacgccttatctatttggcgctggcgcatatg |
| | | | attaagttccgcggtcattttttgatcgagggcgacctgaatccggataa |
| | | | cagcgacgttgacaaactgtttatccagctggtgcagacctataaccagt |
| | | | tgttcgaagagaacccgattaatgcgtctggcgtggacgcgaaggcgatc |
| | | | ctgagcgctcgtctgtccaagtcacgccgcctggagaacttgatcgcgca |
| | | | actgccgggtgagaagaaaaacggcttgttcggcaatctgattgctctgt |
| | | | ccctaggtttgaccccgaatttcaaatctaattttgatctggcggaagat |
| | | | gcgaaattacagttgagcaaggacacttacgatgacgatctggacaacct |
| | | | gctcgctcagatcggcgatcagtatgccgatcttttcctggccgcgaaga |
| | | | acctgagcgatgccatcttgctatcggacatcttacgtgttaacaccgaa |
| | | | atcaccaaagctccgctaagcgcgtccatgattaaacgttacgatgaaca |
| | | | ccaccaggatctgacgctgctgaaagcgttggtgcgccagcaactgccgg |
| | | | aaaaatacaaggaaattttcttcgaccaaagcaagaacggttatgcaggc |
| | | | tatatcgacggtggtgcgtcgcaagaagaattttataaattcatcaaacc |
| | | | gattttggagaagatggatggtaccgaggagctgctggtaaaattgaatc |
| | | | gggaagacctcttgcgaaagcagcgtactttcgataatggtagcatcccg |
| | | | caccagattcatctgggcgaacttcacgcaatcctacgtcgtcaagagga |
| | | | tttctacccgtttctgaaggataaccgtgaaaagatcgagaagattctga |
| | | | cctttcgtattccgtattatgtgggtccgctggcgagaggcaacagccgt |
| | | | tttgcgtggatgacccgcaagtcggaagagactatcacgccgtggaattt |
| | | | cgaggaggtcgtggacaagggtgcatcagcgcaatcttttattgaacgta |
| | | | tgaccaattttgataaaaaccttccaaatgaaaaagtgttgccgaagcac |
| | | | agcttgctgtacgagtatttcaccgtttacaatgaactgacgaaagtcaa |
| | | | gtacgttaccgaaggtatgcgtaagccagcctttctgtctggggagcaaa |
| | | | aaaaagccattgtggatctgctgttcaagaccaatcgcaaggttaccgtc |
| | | | aagcaactgaaagaggattacttcaaaaaaatcgaatgttttgactccgt |
| | | | ggaaatcagcggcgttgaagaccgctttaatgcatctctgggcacctacc |
| | | | acgatctactcaagatcatcaaggataaggacttcctggacaacgaagaa |
| | | | aatgaggatatcttggaagatattgtgctgactttaacgttgttcgagga |
| | | | tcgtgagatgattgaagaacgcctcaaaacgtacgcacatctgtttgacg |
| | | | ataaggtgatgaaacagctaaagcgccgtcgttacaccggttggggtcgt |
| | | | ctgtctagaaagctgattaacggcattcgtgacaaacaatctggcaagac |
| | | | catcctggacttcctcaaatccgacggcttcgcaaatcgtaatttcatgc |
| | | | aactgatccatgatgatagcctgacctttaaagaagatattcagaaggct |
| | | | caagtgtccggccagggtgactcgctgcatgagcatatcgcgaatctggc |
| | | | tggctcccctgcgataaaaaaaggtattctccaaacggtgaaagttgtgg |
| | | | atgaactggttaaagtcatgggtcgtcataaaccggaaaacatcgtgatt |
| | | | gagatggcacgtgagaaccagaccacccagaaaggtcaaaaaaacagtcg |
| | | | tgagcgtatgaaacgtatcgaagaggggattaaggagttaggcagccaga |
| | | | ttctgaaggagcacccggttgagaacacccagctgcaaaacgagaagctc |
| | | | tacctgtattacctgcaaaacggccgtgacatgtatgttgaccaggagct |
| | | | ggacatcaatcgtctcagcgactacgacgtggatgctatcgtgccgcaga |
| | | | gctttctaaaagatgattccatcgacaataaagtgctcacccgtagcgac |
| | | | aagaaccgtggtaaaagcgacaacgtgccgagcgaggaagtagttaagaa |
| | | | aatgaaaaactactggcgtcagctattgaacgccaagcttatcacccagc |
| | | | gtaagtttgacaacttgacgaaggcagagcgtggcggcctgagcgagcta |
| | | | gacaaggcgggttttattaaacgccagctggtggaaactcgccagattac |
| | | | caaacacgttgcgcaaatcttggactcacgcatgaatacgaagtacgacg |
| | | | aaaacgataaattgatccgcgaggtgaaggtcataaccctgaaatctaaa |
| | | | ctggttagcgatttccggaaggactttcaattttataaagtccgggagat |
| | | | taacaactatcaccatgcacacgacgcttacttaaatgcggttgttggca |
| | | | cggccctgatcaagaagtacccgaaactggaaagcgagtttgtttatggt |
| | | | gactacaaggtgtatgacgttcgcaagatgattgcaaaatccgaacaaga |
| | | | gattggtaaggcgacggctaagtattttttctacagcaatatcatgaact |
| | | | tcttcaagactgaaatcaccctggcgaacggtgaaattcgtaaacgtccg |
| | | | ttgatcgaaaccaacggtgaaaccggtgaaatcgtttgggataaaggtag |
| | | | ggattttgcgaccgttcgtaaggtcctgagcatgccgcaggttaacatcg |
| | | | tgaaaaagaccgaagtgcagaccggcggtttctccaaggagagtatctta |
| | | | ccgaagcgcaacagtgacaagctgatcgcgcgtaaaaaagactgggatcc |
| | | | gaaaaaatacggcggctttgactccccgaccgtcgcgtacagcgtactgg |
| | | | tcgtggctaaagtggagaaaggcaaatcgaaaaagctgaaatccgttaaa |
| | | | gagttgctgggtattacgattatggaacgttcgagcttcgagaaaaatcc |
| | | | gatcgacttcctggaggccaaaggctacaaggaagttaaaaaagacctga |
| | | | tcattaaactgccgaagtacagcctgttcgagctcgagaacggtcgcaaa |
| | | | cgcatgctggcgagcgcgggtgagctccaaaaaggcaacgaattggctct |
| | | | gccgagcaagtacgtgaacttcctgtatctggcctcccattatgaaaaac |
| | | | tgaagggtagcccggaagataacgaacaaaagcagctgttcgtggaacaa |
| | | | cataaacactatctggacgagattattgagcaaatcagcgagttcagcaa |
| | | | acgcgtgattctggcggatgctaatctggacaaggttctttcagcctata |
| | | | ataaacaccgtgataagccgatccgtgaacaggcagaaaatattattcac |
| | | | ctgttcaccctgacgaacctgggtgcgccagcagcgtttaaatacttcga |
| | | | cacaacgatcgaccggaagcgttataccagcaccaaagaggttcttgacg |
| | | | ccaccttgattcaccaaagcatcacgggtctgtatgaaacccgtattgac |
| | | | ttgagccagttgggcggagattcagggggaagtggcggaagcggtgggtc |
| | | | tacgaacctgagcgacattattgaaaaagaaactggtaaacagttggtga |
| | | | tccaagaatccattctgatgttaccggaggaagtggaagaggtgatcggc |
| | | | aacaagccggagtccgacatcctggttcacaccgcgtatgatgagtcgac |
| | | | cgatgagaatgttatgctgctgaccagcgacgccccagagtacaaaccgt |
| | | | gggcgctcgtcatccaggatagcaatggcgaaaacaagatcaagatgttg |
| | | | tctggcggtagcatggctccgaaaaagaagcgtaaagtaggtattcatgg |
| | | | tgttccggcatcaggttaa |
| 32 | Base editor | hA3G-CTD-dCasPhi-2 | atgggaagtcatcaccaccaccaccattcaggtgagaacctgtacttcca |
| | | | gagcatggctccgaaaaagaaacgtaaggtgggtatccacggcgttccgg |
| | | | cgatggatcccccaacatttactttcaactttaataacgaaccgtgggtt |
| | | | cgcggtcgccacgaaacctacctgtgctatgaggtggaacgtatgcataa |
| | | | tgatacttgggttctgctgaatcaacgtagaggctttttatgcaaccaag |
| | | | ctccgcataaacatggttttctggaaggccgtcacgccgagctgtgcttc |
| | | | cttgacgtgatcccattctggaaactggacttggaccaggattatcgtgt |
| | | | gacctgtttcaccagctggtcgccgtgcttcagctgtgcgcaggagatgg |
| | | | cgaagttcatctccaaaaacaagcacgttagcttgtgcatttttaccgcg |
| | | | cgtatttacgatgatcaaggtcgctgccaagagggcctgcgtaccctggc |
| | | | agaggctggtgcgaagatctctatcatgacgtacagcgaatttaaacact |
| | | | gctgggacacgtttgtcgatcaccagggttgtccgttccagccgtgggac |
| | | | ggcttggacgaacatagccaggatttgtccggtcgtctgcgcgcaattct |
| | | | acagaatcaagagaactctggaagtgagacacccgggacttcagaatccg |
| | | | cgaccccggagtctggaatgccaaaacccgctgtagagtcagaattttca |
| | | | aaagttttgaaaaagcactttccgggagaacgtttccgctcgagctatat |
| | | | gaaaagaggcggtaagatcctggctgcccaaggcgaagaggcagttgttg |
| | | | cgtatctacagggcaagtccgaggaggagcctccgaacttccagccaccg |
| | | | gcgaagtgccacgtggtgaccaaatcccgtgacttcgcggaatggccgat |
| | | | catgaaggcgtcggaagcgattcaacgttatatctatgcactgtcgacca |
| | | | ccgaacgcgcggcctgtaaaccgggcaaaagtagtgagagccatgcagcg |
| | | | tggttcgccgctaccggtgtcagcaaccacggctacagccacgttcaggg |
| | | | tctgaacctgatttttgatcataccttgggccgttacgatggtgttctga |
| | | | aaaaggtccaactgcgcaacgaaaaggcgagagcgcgtttagagagcatt |
| | | | aacgcgtctagagcagatgaaggtttaccggagattaaagccgaggagga |
| | | | agaagtggctacgaacgaaaccggccacctgttgcaaccgccgggtatca |
| | | | atccgagcttttacgtgtatcagaccattagcccgcaggcataccgtccg |
| | | | cgtgacgagatcgtgctgccgccggagtacgcaggttacgtacgtgaccc |
| | | | gaatgctccgattccgctgggtgtggtgcgtaaccgttgcgatatccaaa |
| | | | aagggtgcccgggctacatcccggaatggcagcgtgaagcaggtaccgcg |
| | | | atcagcccgaaaacaggcaaggccgtaactgtgccgggcctgtctccgaa |
| | | | gaaaaacaaacgtatgcgtaggtactggcgctctgagaaggagaaagctc |
| | | | aagatgcgctgttggttaccgtacgcattggtaccgattgggttgtcatc |
| | | | gacgtgcgcggtctgttgcgcaacgcccgttggcgcaccatcgctcccaa |
| | | | ggacatcagcctgaatgcgctcctcgatttgtttaccggtgatccggtga |
| | | | tcgatgtgcgtcgcaatattgttacctttacgtacaccctcgacgcctgt |
| | | | ggtacctacgcccgtaaatggaccttgaaaggcaaacagacgaaagcgac |
| | | | cctggacaaactgacggccactcagacggttgcgttggttgcgattgcgc |
| | | | ttggtcagaccaacccgatttccgctggcattagccgtgttacacaagaa |
| | | | aatggtgcactgcaatgcgaaccgctggaccggttcaccctgccggatga |
| | | | cctgttgaaggacatcagcgcatatcgcattgcttgggacagaaacgaag |
| | | | aggaactacgcgctcgcagcgtcgaggccctgccggaggcgcaacaagcg |
| | | | gaggtccgtgcgctggacggggtgtccaaagaaaccgcgcgtacccaact |
| | | | gtgcgcagatttcggcctggacccgaaacgtctgccgtgggataaaatga |
| | | | gctccaatactacctttatcagcgaagccttgctgtctaacagcgtgagc |
| | | | cgtgatcaggtttttttcactccggctccgaagaagggtgctaagaaaaa |
| | | | agcgcctgttgaagttatgcgtaaagaccgcacttgggcacgtgcgtaca |
| | | | agccgcgtctgagcgtcgaagctcagaaactgaagaacgaagccctgtgg |
| | | | gcacttaagcgcacctccccggagtatttgaagctgagccgtcgcaaaga |
| | | | ggagctgtgtcgtcgtagcatcaactatgttatcgaaaagacccgtcgcc |
| | | | gtacgcaatgtcagattgtgatcccggtgatcgaggatctgaatgttcgc |
| | | | ttctttcatggttctggtaagagactgcccggttgggataatttcttcac |
| | | | cgctaaaaaagagaaccgttggttcatccagggcctgcacaaagcgttta |
| | | | gcgatctgcgtacgcaccgttccttctatgtttttgaagttcgtccggag |
| | | | cgcacctctattacctgtccaaagtgcggtcattgcgaggtgggtaatcg |
| | | | tgacggtgaggcgtttcagtgcctgagctgcggcaagacgtgcaacgcag |
| | | | atcttgacgtcgcgacccataatctgacccaagtggcgttgaccggtaag |
| | | | accatgccgaagcgcgaggagccgcgtgacgctcagggtacagcgccagc |
| | | | gcgcaagactaagaaggcgagcaaatcaaaggcgccaccggcggagcgcg |
| | | | aagaccagaccccggcgcaagagccaagccagaccagttctggaggtagc |
| | | | gggggtagcggtggtagcactaacttaagtgatattatcgagaaagaaac |
| | | | cgggaaacagctggtgatccaagaaagcattctgatgctgcccgaagaag |
| | | | ttgaagaagtgattggtaacaagcctgagtcggacatcttagtgcacacc |
| | | | gcgtacgatgaaagcacggatgagaatgtgatgctgctcacctctgatgc |
| | | | ccccgaatacaagccgtgggctctggtgatccaggactctaacggagaaa |
| | | | acaaaattaagatgctctctggcggctccatggcgcctaaaaaaaaacgc |
| | | | aaagtggggattcacggggttccggcgtaa |
| 33 | Base editor | hA3G-CTD-dLbCas12a | atgggaagtcatcaccaccaccaccattcaggtgagaacctgtacttcca |
| | | | gagcatggctccgaaaaagaaacgtaaggtgggtatccacggcgttccgg |
| | | | cgatggatcccccaacatttactttcaactttaataacgaaccgtgggtt |
| | | | cgcggtcgccacgaaacctacctgtgctatgaggtggaacgtatgcataa |
| | | | tgatacttgggttctgctgaatcaacgtagaggctttttatgcaaccaag |
| | | | ctccgcataaacatggttttctggaaggccgtcacgccgagctgtgcttc |
| | | | cttgacgtgatcccattctggaaactggacttggaccaggattatcgtgt |
| | | | gacctgtttcaccagctggtcgccgtgcttcagctgtgcgcaggagatgg |
| | | | cgaagttcatctccaaaaacaagcacgttagcttgtgcatttttaccgcg |
| | | | cgtatttacgatgatcaaggtcgctgccaagagggcctgcgtaccctggc |
| | | | agaggctggtgcgaagatctctatcatgacgtacagcgaatttaaacact |
| | | | gctgggacacgtttgtcgatcaccagggttgtccgttccagccgtgggac |
| | | | ggcttggacgaacatagccaggatttgtccggtcgtctgcgcgcaattct |
| | | | acagaatcaagagaactctggaagtgagacacccgggacttcagaatccg |
| | | | cgaccccggagtctggaatgtcaaaactagaaaagtttacaaattgttac |
| | | | tccctgtcgaaaaccctgagattcaaagcgatcccggtgggtaaaactca |
| | | | agagaacattgataacaaacgtctgctggttgaggacgaaaagcgcgcgg |
| | | | aggattacaaaggcgttaaaaagctgttggaccgttattacctgtccttt |
| | | | attaatgatgttctgcacagcattaaattaaagaacctaaacaattacat |
| | | | ctccctcttccgtaaaaagacccgcaccgaaaaggaaaataaagaactgg |
| | | | agaaccttgagattaatctgcgtaaagaaatcgctaaagcgtttaaaggc |
| | | | aacgagggttataaatccttatttaaaaaagacatcatcgaaacgattct |
| | | | cccagaattcctggacgacaaagacgagatcgcgctggttaattccttca |
| | | | acggctttaccaccgcattcaccggcttttttgataaccgcgaaaacatg |
| | | | ttctctgaagaggccaaaagcacctcaattgcgtttcgctgcattaacga |
| | | | aaatctgacgcgttatatctctaacatggatatcttcgagaaggttgacg |
| | | | ctatctttgataaacatgaagtccaggagatcaaggagaagatcctgaac |
| | | | agcgactacgacgtggaggatttcttcgagggtgagttcttcaattttgt |
| | | | tttgacccaagaaggcatcgatgtctacaatgcgataattggtggttttg |
| | | | ttaccgagagcggggagaaaattaagggtctgaacgaatatatcaacttg |
| | | | tacaaccagaaaaccaaacaaaaactgccgaagtttaagccgctgtacaa |
| | | | gcaagtcttgtcggatcgtgaatcgctgagcttttacggcgaaggctaca |
| | | | ccagcgatgaggaggtgttggaggtgttccgcaatacgttgaataagaac |
| | | | tctgaaattttttccagcattaagaagctggaaaaactattcaagaactt |
| | | | cgacgagtatagcagcgcaggtatctttgtgaaaaacggtccggctatta |
| | | | gcaccatctcgaaagacattttcggcgaatggaatgttattcgtgataag |
| | | | tggaatgccgaatacgacgacattcatctgaagaaaaaggcggttgtaac |
| | | | tgaaaagtacgaggacgatcgtcgtaagtcgttcaaaaagatcggctcct |
| | | | tctctctggagcaactgcaagagtacgccgatgccgatttgtcagtcgtg |
| | | | gagaagctgaaagagatcatcattcaaaaagttgacgaaatctataaggt |
| | | | ctatggtagcagtgaaaaactgtttgacgcggacttcgtgctggagaaaa |
| | | | gcctgaagaaaaatgacgccgtggtggcaatcatgaaagatcttctggat |
| | | | agcgtcaagtctttcgaaaactacatcaaagcgtttttcggtgaaggcaa |
| | | | ggaaacgaatcgtgacgagagcttctatggtgatttcgtcctagcctatg |
| | | | atatcctgcttaaggtggatcacatctatgatgccatccgcaactatgtt |
| | | | acccagaaaccgtactcaaaggacaagttcaagttatacttccagaatcc |
| | | | gcagtttatgggtggctgggataaagataaggaaaccgactatcgcgcca |
| | | | ccattttacgttacggtagtaagtactatctggcaatcatggataaaaaa |
| | | | tatgcgaaatgcctgcaaaagatcgacaaggacgatgttaacggcaatta |
| | | | tgagaagatcaactacaaactgctgccgggcccaaataagatgctgccga |
| | | | aggtgtttttcagcaaaaaatggatggcgtattacaatccgagcgaggac |
| | | | atccaaaagatctacaaaaacggtacgtttaagaagggtgatatgttcaa |
| | | | cctcaacgattgtcataaacttattgacttcttcaaggatagcatatctc |
| | | | gttatccgaagtggagcaacgcgtatgactttaactttagcgaaaccgag |
| | | | aaatacaaagatatcgcgggtttctatcgtgaagttgaagaacagggtta |
| | | | caaagtgtccttcgagtccgcgagcaagaaggaggtagacaagctggtcg |
| | | | aggagggtaagttgtacatgtttcagatttacaataaggacttttccgac |
| | | | aagtcccatggtacaccgaatttgcacaccatgtatttcaagcttctgtt |
| | | | cgacgagaacaaccacggccagatccgtctgagcggtggtgcagagctgt |
| | | | tcatgcgtagagctagcctgaagaaagaagagttggtcgttcatccggct |
| | | | aattcgccgatcgcgaacaaaaatccagataaccctaaaaagactacgac |
| | | | cctgagctatgatgtgtataaagacaaacgctttagcgaagaccagtatg |
| | | | agttacacattccgatcgctatcaacaagtgcccgaagaatattttcaag |
| | | | atcaacaccgaagtgcgtgttcttttgaagcacgatgacaacccgtatgt |
| | | | gattggtattgctcgtggtgaacgtaatctgctgtacattgtggttgtgg |
| | | | acggcaaaggaaacatcgtggaacagtacagcctgaacgaaatcattaac |
| | | | aatttcaacggcattcgcattaagacggattaccattctttgctggacaa |
| | | | aaaggagaaagagcgtttcgaggcccgtcagaactggacctccatcgaga |
| | | | acattaaggagctgaaggcgggttacatcagccaggtggtgcacaaaatc |
| | | | tgcgaactggtggagaagtacgacgcggtgattgctttggcggatttgaa |
| | | | ctccggttttaagaacagccgcgttaaagtggaaaagcaggtttatcaga |
| | | | aattcgaaaaaatgctgattgataaattgaattacatggttgacaaaaag |
| | | | tctaacccgtgtgcaaccggcggtgcgctgaagggctaccaaatcaccaa |
| | | | taagtttgaaagctttaagagcatgagcacgcaaaatggcttcatttttt |
| | | | acattccggcgtggctgaccagcaagatcgatccgtcaaccggctttgtt |
| | | | aatttactgaagacgaagtatacctctattgccgactctaaaaaatttat |
| | | | tagttccttcgatagaatcatgtatgttccggaagaggacctgttcgaat |
| | | | ttgcactggactacaagaactttagccgtaccgatgctgattatataaag |
| | | | aaatggaaactctacagctatggcaaccgcatccgtatctttcgcaaccc |
| | | | gaaaaaaaacaacgtgttcgactgggaagaggtatgtctgacttcagcgt |
| | | | acaaggagcttttcaataaatatggtatcaactaccagcagggtgatatc |
| | | | cgtgcactgctttgcgagcaaagcgataaagcgttctattcaagcttcat |
| | | | ggcgctgatgagcctgatgctgcaaatgcgcaacagcatcaccggtcgta |
| | | | ccgatgttgcatttctgatttctccagtaaaaaacagcgatggtatcttt |
| | | | tacgacagccgtaattacgaggcccaagaaaatgcgattttgccgaagaa |
| | | | cgctgacgcgaatggtgcatacaacattgcaaggaaagttctgtgggcaa |
| | | | tcggccaatttaaaaaagcggaagatgagaagctggacaaagtgaaaatt |
| | | | gcgattagcaacaaggagtggctggagtatgcgcagacctccgttaaatc |
| | | | agggggaagtggcggaagcggtgggtctacgaacctgagcgacattattg |
| | | | aaaaagaaactggtaaacagttggtgatccaagaatccattctgatgtta |
| | | | ccggaggaagtggaagaggtgatcggcaacaagccggagtccgacatcct |
| | | | ggttcacaccgcgtatgatgagtcgaccgatgagaatgttatgctgctga |
| | | | ccagcgacgccccagagtacaaaccgtgggcgctcgtcatccaggatagc |
| | | | aatggcgaaaacaagatcaagatgttgtctggcggtagcatggctccgaa |
| | | | aaagaagcgtaaagtaggtattcatggtgttccggcatcaggttaa |
| 34 | Base editor | minStart-dCas9 | atgggcagccaccaccaccaccaccacagcggcgagaacctgtacttcca |
| | | | gtccatggctccaaagaagaagagaaaggttggaatccacggagttccag |
| | | | cttctggaatggataaaaagtattcaataggactagctattggcaccaac |
| | | | agcgttggttgggcagttatcaccgatgaatataaggtcccgagcaagaa |
| | | | gtttaaggtcctgggcaacaccgatcgccacagcataaaaaagaacctta |
| | | | ttggtgcgctgctgttcgattctggtgagactgccgaagcgacccgtctg |
| | | | aagagaaccgcgcgtcgtagatacacccggcgcaagaaccgtatctgcta |
| | | | tctgcaagaaattttcagcaacgagatggctaaggtcgacgactccttct |
| | | | ttcatcgcctggaggaaagctttctggttgaggaagacaaaaagcacgag |
| | | | cgtcatccgattttcggtaacattgttgatgaagttgcctaccacgaaaa |
| | | | ataccccacaatttatcatctgcgtaaaaaactggtagactccaccgata |
| | | | aggcggatttacgccttatctatttggcgctggcgcatatgattaagttc |
| | | | cgcggtcattttttgatcgagggcgacctgaatccggataacagcgacgt |
| | | | tgacaaactgtttatccagctggtgcagacctataaccagttgttcgaag |
| | | | agaacccgattaatgcgtctggcgtggacgcgaaggcgatcctgagcgct |
| | | | cgtctgtccaagtcacgccgcctggagaacttgatcgcgcaactgccggg |
| | | | tgagaagaaaaacggcttgttcggcaatctgattgctctgtccctaggtt |
| | | | tgaccccgaatttcaaatctaattttgatctggcggaagatgcgaaatta |
| | | | cagttgagcaaggacacttacgatgacgatctggacaacctgctcgctca |
| | | | gatcggcgatcagtatgccgatcttttcctggccgcgaagaacctgagcg |
| | | | atgccatcttgctatcggacatcttacgtgttaacaccgaaatcaccaaa |
| | | | gctccgctaagcgcgtccatgattaaacgttacgatgaacaccaccagga |
| | | | tctgacgctgctgaaagcgttggtgcgccagcaactgccggaaaaataca |
| | | | aggaaattttcttcgaccaaagcaagaacggttatgcaggctatatcgac |
| | | | ggtggtgcgtcgcaagaagaattttataaattcatcaaaccgattttgga |
| | | | gaagatggatggtaccgaggagctgctggtaaaattgaatcgggaagacc |
| | | | tcttgcgaaagcagcgtactttcgataatggtagcatcccgcaccagatt |
| | | | catctgggcgaacttcacgcaatcctacgtcgtcaagaggatttctaccc |
| | | | gtttctgaaggataaccgtgaaaagatcgagaagattctgacctttcgta |
| | | | ttccgtattatgtgggtccgctggcgagaggcaacagccgttttgcgtgg |
| | | | atgacccgcaagtcggaagagactatcacgccgtggaatttcgaggaggt |
| | | | cgtggacaagggtgcatcagcgcaatcttttattgaacgtatgaccaatt |
| | | | ttgataaaaaccttccaaatgaaaaagtgttgccgaagcacagcttgctg |
| | | | tacgagtatttcaccgtttacaatgaactgacgaaagtcaagtacgttac |
| | | | cgaaggtatgcgtaagccagcctttctgtctggggagcaaaaaaaagcca |
| | | | ttgtggatctgctgttcaagaccaatcgcaaggttaccgtcaagcaactg |
| | | | aaagaggattacttcaaaaaaatcgaatgttttgactccgtggaaatcag |
| | | | cggcgttgaagaccgctttaatgcatctctgggcacctaccacgatctac |
| | | | tcaagatcatcaaggataaggacttcctggacaacgaagaaaatgaggat |
| | | | atcttggaagatattgtgctgactttaacgttgttcgaggatcgtgagat |
| | | | gattgaagaacgcctcaaaacgtacgcacatctgtttgacgataaggtga |
| | | | tgaaacagctaaagcgccgtcgttacaccggttggggtcgtctgtctaga |
| | | | aagctgattaacggcattcgtgacaaacaatctggcaagaccatcctgga |
| | | | cttcctcaaatccgacggcttcgcaaatcgtaatttcatgcaactgatcc |
| | | | atgatgatagcctgacctttaaagaagatattcagaaggctcaagtgtcc |
| | | | ggccagggtgactcgctgcatgagcatatcgcgaatctggctggctcccc |
| | | | tgcgataaaaaaaggtattctccaaacggtgaaagttgtggatgaactgg |
| | | | ttaaagtcatgggtcgtcataaaccggaaaacatcgtgattgagatggca |
| | | | cgtgagaaccagaccacccagaaaggtcaaaaaaacagtcgtgagcgtat |
| | | | gaaacgtatcgaagaggggattaaggagttaggcagccagattctgaagg |
| | | | agcacccggttgagaacacccagctgcaaaacgagaagctctacctgtat |
| | | | tacctgcaaaacggccgtgacatgtatgttgaccaggagctggacatcaa |
| | | | tcgtctcagcgactacgacgtggatgctatcgtgccgcagagctttctaa |
| | | | aagatgattccatcgacaataaagtgctcacccgtagcgacaagaaccgt |
| | | | ggtaaaagcgacaacgtgccgagcgaggaagtagttaagaaaatgaaaaa |
| | | | ctactggcgtcagctattgaacgccaagcttatcacccagcgtaagtttg |
| | | | acaacttgacgaaggcagagcgtggcggcctgagcgagctagacaaggcg |
| | | | ggttttattaaacgccagctggtggaaactcgccagattaccaaacacgt |
| | | | tgcgcaaatcttggactcacgcatgaatacgaagtacgacgaaaacgata |
| | | | aattgatccgcgaggtgaaggtcataaccctgaaatctaaactggttagc |
| | | | gatttccggaaggactttcaattttataaagtccgggagattaacaacta |
| | | | tcaccatgcacacgacgcttacttaaatgcggttgttggcacggccctga |
| | | | tcaagaagtacccgaaactggaaagcgagtttgtttatggtgactacaag |
| | | | gtgtatgacgttcgcaagatgattgcaaaatccgaacaagagattggtaa |
| | | | ggcgacggctaagtattttttctacagcaatatcatgaacttcttcaaga |
| | | | ctgaaatcaccctggcgaacggtgaaattcgtaaacgtccgttgatcgaa |
| | | | accaacggtgaaaccggtgaaatcgtttgggataaaggtagggattttgc |
| | | | gaccgttcgtaaggtcctgagcatgccgcaggttaacatcgtgaaaaaga |
| | | | ccgaagtgcagaccggcggtttctccaaggagagtatcttaccgaagcgc |
| | | | aacagtgacaagctgatcgcgcgtaaaaaagactgggatccgaaaaaata |
| | | | cggcggctttgactccccgaccgtcgcgtacagcgtactggtcgtggcta |
| | | | aagtggagaaaggcaaatcgaaaaagctgaaatccgttaaagagttgctg |
| | | | ggtattacgattatggaacgttcgagcttcgagaaaaatccgatcgactt |
| | | | cctggaggccaaaggctacaaggaagttaaaaaagacctgatcattaaac |
| | | | tgccgaagtacagcctgttcgagctcgagaacggtcgcaaacgcatgctg |
| | | | gcgagcgcgggtgagctccaaaaaggcaacgaattggctctgccgagcaa |
| | | | gtacgtgaacttcctgtatctggcctcccattatgaaaaactgaagggta |
| | | | gcccggaagataacgaacaaaagcagctgttcgtggaacaacataaacac |
| | | | tatctggacgagattattgagcaaatcagcgagttcagcaaacgcgtgat |
| | | | tctggcggatgctaatctggacaaggttctttcagcctataataaacacc |
| | | | gtgataagccgatccgtgaacaggcagaaaatattattcacctgttcacc |
| | | | ctgacgaacctgggtgcgccagcagcgtttaaatacttcgacacaacgat |
| | | | cgaccggaagcgttataccagcaccaaagaggttcttgacgccaccttga |
| | | | ttcaccaaagcatcacgggtctgtatgaaacccgtattgacttgagccag |
| | | | ttgggcggagattcagggggaagtggcggaagcggtgggtctacgaacct |
| | | | gagcgacattattgaaaaagaaactggtaaacagttggtgatccaagaat |
| | | | ccattctgatgttaccggaggaagtggaagaggtgatcggcaacaagccg |
| | | | gagtccgacatcctggttcacaccgcgtatgatgagtcgaccgatgagaa |
| | | | tgttatgctgctgaccagcgacgccccagagtacaaaccgtgggcgctcg |
| | | | tcatccaggatagcaatggcgaaaacaagatcaagatgttgtctggcggt |
| | | | agcatggctccgaaaaagaagcgtaaagtaggtattcatggtgttccggc |
| | | | atcaggttaa |
| 35 | Base editor | minStart-dCasPhi-2 | atgggcagccaccaccaccaccaccacagcggcgagaacctgtacttcca |
| | | | gtccatggctccaaagaagaagagaaaggttggaatccacggagttccag |
| | | | cttctggaatgccaaaacccgctgtagagtcagaattttcaaaagttttg |
| | | | aaaaagcactttccgggagaacgtttccgctcgagctatatgaaaagagg |
| | | | cggtaagatcctggctgcccaaggcgaagaggcagttgttgcgtatctac |
| | | | agggcaagtccgaggaggagcctccgaacttccagccaccggcgaagtgc |
| | | | cacgtggtgaccaaatcccgtgacttcgcggaatggccgatcatgaaggc |
| | | | gtcggaagcgattcaacgttatatctatgcactgtcgaccaccgaacgcg |
| | | | cggcctgtaaaccgggcaaaagtagtgagagccatgcagcgtggttcgcc |
| | | | gctaccggtgtcagcaaccacggctacagccacgttcagggtctgaacct |
| | | | gatttttgatcataccttgggccgttacgatggtgttctgaaaaaggtcc |
| | | | aactgcgcaacgaaaaggcgagagcgcgtttagagagcattaacgcgtct |
| | | | agagcagatgaaggtttaccggagattaaagccgaggaggaagaagtggc |
| | | | tacgaacgaaaccggccacctgttgcaaccgccgggtatcaatccgagct |
| | | | tttacgtgtatcagaccattagcccgcaggcataccgtccgcgtgacgag |
| | | | atcgtgctgccgccggagtacgcaggttacgtacgtgacccgaatgctcc |
| | | | gattccgctgggtgtggtgcgtaaccgttgcgatatccaaaaagggtgcc |
| | | | cgggctacatcccggaatggcagcgtgaagcaggtaccgcgatcagcccg |
| | | | aaaacaggcaaggccgtaactgtgccgggcctgtctccgaagaaaaacaa |
| | | | acgtatgcgtaggtactggcgctctgagaaggagaaagctcaagatgcgc |
| | | | tgttggttaccgtacgcattggtaccgattgggttgtcatcgacgtgcgc |
| | | | ggtctgttgcgcaacgcccgttggcgcaccatcgctcccaaggacatcag |
| | | | cctgaatgcgctcctcgatttgtttaccggtgatccggtgatcgatgtgc |
| | | | gtcgcaatattgttacctttacgtacaccctcgacgcctgtggtacctac |
| | | | gcccgtaaatggaccttgaaaggcaaacagacgaaagcgaccctggacaa |
| | | | actgacggccactcagacggttgcgttggttgcgattgcgcttggtcaga |
| | | | ccaacccgatttccgctggcattagccgtgttacacaagaaaatggtgca |
| | | | ctgcaatgcgaaccgctggaccggttcaccctgccggatgacctgttgaa |
| | | | ggacatcagcgcatatcgcattgcttgggacagaaacgaagaggaactac |
| | | | gcgctcgcagcgtcgaggccctgccggaggcgcaacaagcggaggtccgt |
| | | | gcgctggacggggtgtccaaagaaaccgcgcgtacccaactgtgcgcaga |
| | | | tttcggcctggacccgaaacgtctgccgtgggataaaatgagctccaata |
| | | | ctacctttatcagcgaagccttgctgtctaacagcgtgagccgtgatcag |
| | | | gtttttttcactccggctccgaagaagggtgctaagaaaaaagcgcctgt |
| | | | tgaagttatgcgtaaagaccgcacttgggcacgtgcgtacaagccgcgtc |
| | | | tgagcgtcgaagctcagaaactgaagaacgaagccctgtgggcacttaag |
| | | | cgcacctccccggagtatttgaagctgagccgtcgcaaagaggagctgtg |
| | | | tcgtcgtagcatcaactatgttatcgaaaagacccgtcgccgtacgcaat |
| | | | gtcagattgtgatcccggtgatcgaggatctgaatgttcgcttctttcat |
| | | | ggttctggtaagagactgcccggttgggataatttcttcaccgctaaaaa |
| | | | agagaaccgttggttcatccagggcctgcacaaagcgtttagcgatctgc |
| | | | gtacgcaccgttccttctatgtttttgaagttcgtccggagcgcacctct |
| | | | attacctgtccaaagtgcggtcattgcgaggtgggtaatcgtgacggtga |
| | | | ggcgtttcagtgcctgagctgcggcaagacgtgcaacgcagatcttgacg |
| | | | tcgcgacccataatctgacccaagtggcgttgaccggtaagaccatgccg |
| | | | aagcgcgaggagccgcgtgacgctcagggtacagcgccagcgcgcaagac |
| | | | taagaaggcgagcaaatcaaaggcgccaccggcggagcgcgaagaccaga |
| | | | ccccggcgcaagagccaagccagaccagttctggaggtagcgggggtagc |
| | | | ggtggtagcactaacttaagtgatattatcgagaaagaaaccgggaaaca |
| | | | gctggtgatccaagaaagcattctgatgctgcccgaagaagttgaagaag |
| | | | tgattggtaacaagcctgagtcggacatcttagtgcacaccgcgtacgat |
| | | | gaaagcacggatgagaatgtgatgctgctcacctctgatgcccccgaata |
| | | | caagccgtgggctctggtgatccaggactctaacggagaaaacaaaatta |
| | | | agatgctctctggcggctccatggcgcctaaaaaaaaacgcaaagtgggg |
| | | | attcacggggttccggcgtaa |
| 36 | Base editor | minStart-dLbCas12a | atgggcagccaccaccaccaccaccacagcggcgagaacctgtacttcca |
| | | | gtccatggctccaaagaagaagagaaaggttggaatccacggagttccag |
| | | | cttctggaatgtcaaaactagaaaagtttacaaattgttactccctgtcg |
| | | | aaaaccctgagattcaaagcgatcccggtgggtaaaactcaagagaacat |
| | | | tgataacaaacgtctgctggttgaggacgaaaagcgcgcggaggattaca |
| | | | aaggcgttaaaaagctgttggaccgttattacctgtcctttattaatgat |
| | | | gttctgcacagcattaaattaaagaacctaaacaattacatctccctctt |
| | | | ccgtaaaaagacccgcaccgaaaaggaaaataaagaactggagaaccttg |
| | | | agattaatctgcgtaaagaaatcgctaaagcgtttaaaggcaacgagggt |
| | | | tataaatccttatttaaaaaagacatcatcgaaacgattctcccagaatt |
| | | | cctggacgacaaagacgagatcgcgctggttaattccttcaacggcttta |
| | | | ccaccgcattcaccggcttttttgataaccgcgaaaacatgttctctgaa |
| | | | gaggccaaaagcacctcaattgcgtttcgctgcattaacgaaaatctgac |
| | | | gcgttatatctctaacatggatatcttcgagaaggttgacgctatctttg |
| | | | ataaacatgaagtccaggagatcaaggagaagatcctgaacagcgactac |
| | | | gacgtggaggatttcttcgagggtgagttcttcaattttgttttgaccca |
| | | | agaaggcatcgatgtctacaatgcgataattggtggttttgttaccgaga |
| | | | gcggggagaaaattaagggtctgaacgaatatatcaacttgtacaaccag |
| | | | aaaaccaaacaaaaactgccgaagtttaagccgctgtacaagcaagtctt |
| | | | gtcggatcgtgaatcgctgagcttttacggcgaaggctacaccagcgatg |
| | | | aggaggtgttggaggtgttccgcaatacgttgaataagaactctgaaatt |
| | | | ttttccagcattaagaagctggaaaaactattcaagaacttcgacgagta |
| | | | tagcagcgcaggtatctttgtgaaaaacggtccggctattagcaccatct |
| | | | cgaaagacattttcggcgaatggaatgttattcgtgataagtggaatgcc |
| | | | gaatacgacgacattcatctgaagaaaaaggcggttgtaactgaaaagta |
| | | | cgaggacgatcgtcgtaagtcgttcaaaaagatcggctccttctctctgg |
| | | | agcaactgcaagagtacgccgatgccgatttgtcagtcgtggagaagctg |
| | | | aaagagatcatcattcaaaaagttgacgaaatctataaggtctatggtag |
| | | | cagtgaaaaactgtttgacgcggacttcgtgctggagaaaagcctgaaga |
| | | | aaaatgacgccgtggtggcaatcatgaaagatcttctggatagcgtcaag |
| | | | tctttcgaaaactacatcaaagcgtttttcggtgaaggcaaggaaacgaa |
| | | | tcgtgacgagagcttctatggtgatttcgtcctagcctatgatatcctgc |
| | | | ttaaggtggatcacatctatgatgccatccgcaactatgttacccagaaa |
| | | | ccgtactcaaaggacaagttcaagttatacttccagaatccgcagtttat |
| | | | gggtggctgggataaagataaggaaaccgactatcgcgccaccattttac |
| | | | gttacggtagtaagtactatctggcaatcatggataaaaaatatgcgaaa |
| | | | tgcctgcaaaagatcgacaaggacgatgttaacggcaattatgagaagat |
| | | | caactacaaactgctgccgggcccaaataagatgctgccgaaggtgtttt |
| | | | tcagcaaaaaatggatggcgtattacaatccgagcgaggacatccaaaag |
| | | | atctacaaaaacggtacgtttaagaagggtgatatgttcaacctcaacga |
| | | | ttgtcataaacttattgacttcttcaaggatagcatatctcgttatccga |
| | | | agtggagcaacgcgtatgactttaactttagcgaaaccgagaaatacaaa |
| | | | gatatcgcgggtttctatcgtgaagttgaagaacagggttacaaagtgtc |
| | | | cttcgagtccgcgagcaagaaggaggtagacaagctggtcgaggagggta |
| | | | agttgtacatgtttcagatttacaataaggacttttccgacaagtcccat |
| | | | ggtacaccgaatttgcacaccatgtatttcaagcttctgttcgacgagaa |
| | | | caaccacggccagatccgtctgagcggtggtgcagagctgttcatgcgta |
| | | | gagctagcctgaagaaagaagagttggtcgttcatccggctaattcgccg |
| | | | atcgcgaacaaaaatccagataaccctaaaaagactacgaccctgagcta |
| | | | tgatgtgtataaagacaaacgctttagcgaagaccagtatgagttacaca |
| | | | ttccgatcgctatcaacaagtgcccgaagaatattttcaagatcaacacc |
| | | | gaagtgcgtgttcttttgaagcacgatgacaacccgtatgtgattggtat |
| | | | tgctcgtggtgaacgtaatctgctgtacattgtggttgtggacggcaaag |
| | | | gaaacatcgtggaacagtacagcctgaacgaaatcattaacaatttcaac |
| | | | ggcattcgcattaagacggattaccattctttgctggacaaaaaggagaa |
| | | | agagcgtttcgaggcccgtcagaactggacctccatcgagaacattaagg |
| | | | agctgaaggcgggttacatcagccaggtggtgcacaaaatctgcgaactg |
| | | | gtggagaagtacgacgcggtgattgctttggcggatttgaactccggttt |
| | | | taagaacagccgcgttaaagtggaaaagcaggtttatcagaaattcgaaa |
| | | | aaatgctgattgataaattgaattacatggttgacaaaaagtctaacccg |
| | | | tgtgcaaccggcggtgcgctgaagggctaccaaatcaccaataagtttga |
| | | | aagctttaagagcatgagcacgcaaaatggcttcattttttacattccgg |
| | | | cgtggctgaccagcaagatcgatccgtcaaccggctttgttaatttactg |
| | | | aagacgaagtatacctctattgccgactctaaaaaatttattagttcctt |
| | | | cgatagaatcatgtatgttccggaagaggacctgttcgaatttgcactgg |
| | | | actacaagaactttagccgtaccgatgctgattatataaagaaatggaaa |
| | | | ctctacagctatggcaaccgcatccgtatctttcgcaacccgaaaaaaaa |
| | | | caacgtgttcgactgggaagaggtatgtctgacttcagcgtacaaggagc |
| | | | ttttcaataaatatggtatcaactaccagcagggtgatatccgtgcactg |
| | | | ctttgcgagcaaagcgataaagcgttctattcaagcttcatggcgctgat |
| | | | gagcctgatgctgcaaatgcgcaacagcatcaccggtcgtaccgatgttg |
| | | | catttctgatttctccagtaaaaaacagcgatggtatcttttacgacagc |
| | | | cgtaattacgaggcccaagaaaatgcgattttgccgaagaacgctgacgc |
| | | | gaatggtgcatacaacattgcaaggaaagttctgtgggcaatcggccaat |
| | | | ttaaaaaagcggaagatgagaagctggacaaagtgaaaattgcgattagc |
| | | | aacaaggagtggctggagtatgcgcagacctccgttaaatcagggggaag |
| | | | tggcggaagcggtgggtctacgaacctgagcgacattattgaaaaagaaa |
| | | | ctggtaaacagttggtgatccaagaatccattctgatgttaccggaggaa |
| | | | gtggaagaggtgatcggcaacaagccggagtccgacatcctggttcacac |
| | | | cgcgtatgatgagtcgaccgatgagaatgttatgctgctgaccagcgacg |
| | | | ccccagagtacaaaccgtgggcgctcgtcatccaggatagcaatggcgaa |
| | | | aacaagatcaagatgttgtctggcggtagcatggctccgaaaaagaagcg |
| | | | taaagtaggtattcatggtgttccggcatcaggttaa |
| 37 | Base editor | mTadA8e-dCas9 | atgggaagtcatcaccaccaccaccattcaggtgagaacctgtacttcca |
| | | | gagcatggctccgaaaaagaaacgtaaggtgggtatccacggcgttccgg |
| | | | cgagcggattagtaatgcaaaattataggctaatagacgctacgttgtat |
| | | | gtgacctttgaaccgtgcgttatgtgcgctggtgcaatgattcattctcg |
| | | | tatcggccgtgtcgtgttcggcgtgagaaacagcaagcgcggtgcggcgg |
| | | | gtagcctgatgaatgttctgaactacccgggtatgaaccaccgtgttgaa |
| | | | attaccgagggtatcttggcagacgagtgcgccgccctgctgtgtgattt |
| | | | ctaccgcatgccgcgtcaggtttttaacgcgcagaaaaaggcgcaaagca |
| | | | gcatcaatagcggcggtagctctggcggctcctccggttcggagtctgga |
| | | | agtgagacacccgggacttcagaatccgcgaccccggagtctggaatgga |
| | | | taaaaagtattcaataggactagctattggcaccaacagcgttggttggg |
| | | | cagttatcaccgatgaatataaggtcccgagcaagaagtttaaggtcctg |
| | | | ggcaacaccgatcgccacagcataaaaaagaaccttattggtgcgctgct |
| | | | gttcgattctggtgagactgccgaagcgacccgtctgaagagaaccgcgc |
| | | | gtcgtagatacacccggcgcaagaaccgtatctgctatctgcaagaaatt |
| | | | ttcagcaacgagatggctaaggtcgacgactccttctttcatcgcctgga |
| | | | ggaaagctttctggttgaggaagacaaaaagcacgagcgtcatccgattt |
| | | | tcggtaacattgttgatgaagttgcctaccacgaaaaataccccacaatt |
| | | | tatcatctgcgtaaaaaactggtagactccaccgataaggcggatttacg |
| | | | ccttatctatttggcgctggcgcatatgattaagttccgcggtcattttt |
| | | | tgatcgagggcgacctgaatccggataacagcgacgttgacaaactgttt |
| | | | atccagctggtgcagacctataaccagttgttcgaagagaacccgattaa |
| | | | tgcgtctggcgtggacgcgaaggcgatcctgagcgctcgtctgtccaagt |
| | | | cacgccgcctggagaacttgatcgcgcaactgccgggtgagaagaaaaac |
| | | | ggcttgttcggcaatctgattgctctgtccctaggtttgaccccgaattt |
| | | | caaatctaattttgatctggcggaagatgcgaaattacagttgagcaagg |
| | | | acacttacgatgacgatctggacaacctgctcgctcagatcggcgatcag |
| | | | tatgccgatcttttcctggccgcgaagaacctgagcgatgccatcttgct |
| | | | atcggacatcttacgtgttaacaccgaaatcaccaaagctccgctaagcg |
| | | | cgtccatgattaaacgttacgatgaacaccaccaggatctgacgctgctg |
| | | | aaagcgttggtgcgccagcaactgccggaaaaatacaaggaaattttctt |
| | | | cgaccaaagcaagaacggttatgcaggctatatcgacggtggtgcgtcgc |
| | | | aagaagaattttataaattcatcaaaccgattttggagaagatggatggt |
| | | | accgaggagctgctggtaaaattgaatcgggaagacctcttgcgaaagca |
| | | | gcgtactttcgataatggtagcatcccgcaccagattcatctgggcgaac |
| | | | ttcacgcaatcctacgtcgtcaagaggatttctacccgtttctgaaggat |
| | | | aaccgtgaaaagatcgagaagattctgacctttcgtattccgtattatgt |
| | | | gggtccgctggcgagaggcaacagccgttttgcgtggatgacccgcaagt |
| | | | cggaagagactatcacgccgtggaatttcgaggaggtcgtggacaagggt |
| | | | gcatcagcgcaatcttttattgaacgtatgaccaattttgataaaaacct |
| | | | tccaaatgaaaaagtgttgccgaagcacagcttgctgtacgagtatttca |
| | | | ccgtttacaatgaactgacgaaagtcaagtacgttaccgaaggtatgcgt |
| | | | aagccagcctttctgtctggggagcaaaaaaaagccattgtggatctgct |
| | | | gttcaagaccaatcgcaaggttaccgtcaagcaactgaaagaggattact |
| | | | tcaaaaaaatcgaatgttttgactccgtggaaatcagcggcgttgaagac |
| | | | cgctttaatgcatctctgggcacctaccacgatctactcaagatcatcaa |
| | | | ggataaggacttcctggacaacgaagaaaatgaggatatcttggaagata |
| | | | ttgtgctgactttaacgttgttcgaggatcgtgagatgattgaagaacgc |
| | | | ctcaaaacgtacgcacatctgtttgacgataaggtgatgaaacagctaaa |
| | | | gcgccgtcgttacaccggttggggtcgtctgtctagaaagctgattaacg |
| | | | gcattcgtgacaaacaatctggcaagaccatcctggacttcctcaaatcc |
| | | | gacggcttcgcaaatcgtaatttcatgcaactgatccatgatgatagcct |
| | | | gacctttaaagaagatattcagaaggctcaagtgtccggccagggtgact |
| | | | cgctgcatgagcatatcgcgaatctggctggctcccctgcgataaaaaaa |
| | | | ggtattctccaaacggtgaaagttgtggatgaactggttaaagtcatggg |
| | | | tcgtcataaaccggaaaacatcgtgattgagatggcacgtgagaaccaga |
| | | | ccacccagaaaggtcaaaaaaacagtcgtgagcgtatgaaacgtatcgaa |
| | | | gaggggattaaggagttaggcagccagattctgaaggagcacccggttga |
| | | | gaacacccagctgcaaaacgagaagctctacctgtattacctgcaaaacg |
| | | | gccgtgacatgtatgttgaccaggagctggacatcaatcgtctcagcgac |
| | | | tacgacgtggatgctatcgtgccgcagagctttctaaaagatgattccat |
| | | | cgacaataaagtgctcacccgtagcgacaagaaccgtggtaaaagcgaca |
| | | | acgtgccgagcgaggaagtagttaagaaaatgaaaaactactggcgtcag |
| | | | ctattgaacgccaagcttatcacccagcgtaagtttgacaacttgacgaa |
| | | | ggcagagcgtggcggcctgagcgagctagacaaggcgggttttattaaac |
| | | | gccagctggtggaaactcgccagattaccaaacacgttgcgcaaatcttg |
| | | | gactcacgcatgaatacgaagtacgacgaaaacgataaattgatccgcga |
| | | | ggtgaaggtcataaccctgaaatctaaactggttagcgatttccggaagg |
| | | | actttcaattttataaagtccgggagattaacaactatcaccatgcacac |
| | | | gacgcttacttaaatgcggttgttggcacggccctgatcaagaagtaccc |
| | | | gaaactggaaagcgagtttgtttatggtgactacaaggtgtatgacgttc |
| | | | gcaagatgattgcaaaatccgaacaagagattggtaaggcgacggctaag |
| | | | tattttttctacagcaatatcatgaacttcttcaagactgaaatcaccct |
| | | | ggcgaacggtgaaattcgtaaacgtccgttgatcgaaaccaacggtgaaa |
| | | | ccggtgaaatcgtttgggataaaggtagggattttgcgaccgttcgtaag |
| | | | gtcctgagcatgccgcaggttaacatcgtgaaaaagaccgaagtgcagac |
| | | | cggcggtttctccaaggagagtatcttaccgaagcgcaacagtgacaagc |
| | | | tgatcgcgcgtaaaaaagactgggatccgaaaaaatacggcggctttgac |
| | | | tccccgaccgtcgcgtacagcgtactggtcgtggctaaagtggagaaagg |
| | | | caaatcgaaaaagctgaaatccgttaaagagttgctgggtattacgatta |
| | | | tggaacgttcgagcttcgagaaaaatccgatcgacttcctggaggccaaa |
| | | | ggctacaaggaagttaaaaaagacctgatcattaaactgccgaagtacag |
| | | | cctgttcgagctcgagaacggtcgcaaacgcatgctggcgagcgcgggtg |
| | | | agctccaaaaaggcaacgaattggctctgccgagcaagtacgtgaacttc |
| | | | ctgtatctggcctcccattatgaaaaactgaagggtagcccggaagataa |
| | | | cgaacaaaagcagctgttcgtggaacaacataaacactatctggacgaga |
| | | | ttattgagcaaatcagcgagttcagcaaacgcgtgattctggcggatgct |
| | | | aatctggacaaggttctttcagcctataataaacaccgtgataagccgat |
| | | | ccgtgaacaggcagaaaatattattcacctgttcaccctgacgaacctgg |
| | | | gtgcgccagcagcgtttaaatacttcgacacaacgatcgaccggaagcgt |
| | | | tataccagcaccaaagaggttcttgacgccaccttgattcaccaaagcat |
| | | | cacgggtctgtatgaaacccgtattgacttgagccagttgggcggagatt |
| | | | cagggggaagtggcggaagcggtgggtctacgaacctgagcgacattatt |
| | | | gaaaaagaaactggtaaacagttggtgatccaagaatccattctgatgtt |
| | | | accggaggaagtggaagaggtgatcggcaacaagccggagtccgacatcc |
| | | | tggttcacaccgcgtatgatgagtcgaccgatgagaatgttatgctgctg |
| | | | accagcgacgccccagagtacaaaccgtgggcgctcgtcatccaggatag |
| | | | caatggcgaaaacaagatcaagatgttgtctggcggtagcatggctccga |
| | | | aaaagaagcgtaaagtaggtattcatggtgttccggcatcaggttaa |
| 38 | Base editor | mTadA8e-dCasPhi-2 | atgggaagtcatcaccaccaccaccattcaggtgagaacctgtacttcca |
| | | | gagcatggctccgaaaaagaaacgtaaggtgggtatccacggcgttccgg |
| | | | cgagcggattagtaatgcaaaattataggctaatagacgctacgttgtat |
| | | | gtgacctttgaaccgtgcgttatgtgcgctggtgcaatgattcattctcg |
| | | | tatcggccgtgtcgtgttcggcgtgagaaacagcaagcgcggtgcggcgg |
| | | | gtagcctgatgaatgttctgaactacccgggtatgaaccaccgtgttgaa |
| | | | attaccgagggtatcttggcagacgagtgcgccgccctgctgtgtgattt |
| | | | ctaccgcatgccgcgtcaggtttttaacgcgcagaaaaaggcgcaaagca |
| | | | gcatcaatagcggcggtagctctggcggctcctccggttcggagtctgga |
| | | | agtgagacacccgggacttcagaatccgcgaccccggagtctggaatgcc |
| | | | aaaacccgctgtagagtcagaattttcaaaagttttgaaaaagcactttc |
| | | | cgggagaacgtttccgctcgagctatatgaaaagaggcggtaagatcctg |
| | | | gctgcccaaggcgaagaggcagttgttgcgtatctacagggcaagtccga |
| | | | ggaggagcctccgaacttccagccaccggcgaagtgccacgtggtgacca |
| | | | aatcccgtgacttcgcggaatggccgatcatgaaggcgtcggaagcgatt |
| | | | caacgttatatctatgcactgtcgaccaccgaacgcgcggcctgtaaacc |
| | | | gggcaaaagtagtgagagccatgcagcgtggttcgccgctaccggtgtca |
| | | | gcaaccacggctacagccacgttcagggtctgaacctgatttttgatcat |
| | | | accttgggccgttacgatggtgttctgaaaaaggtccaactgcgcaacga |
| | | | aaaggcgagagcgcgtttagagagcattaacgcgtctagagcagatgaag |
| | | | gtttaccggagattaaagccgaggaggaagaagtggctacgaacgaaacc |
| | | | ggccacctgttgcaaccgccgggtatcaatccgagcttttacgtgtatca |
| | | | gaccattagcccgcaggcataccgtccgcgtgacgagatcgtgctgccgc |
| | | | cggagtacgcaggttacgtacgtgacccgaatgctccgattccgctgggt |
| | | | gtggtgcgtaaccgttgcgatatccaaaaagggtgcccgggctacatccc |
| | | | ggaatggcagcgtgaagcaggtaccgcgatcagcccgaaaacaggcaagg |
| | | | ccgtaactgtgccgggcctgtctccgaagaaaaacaaacgtatgcgtagg |
| | | | tactggcgctctgagaaggagaaagctcaagatgcgctgttggttaccgt |
| | | | acgcattggtaccgattgggttgtcatcgacgtgcgcggtctgttgcgca |
| | | | acgcccgttggcgcaccatcgctcccaaggacatcagcctgaatgcgctc |
| | | | ctcgatttgtttaccggtgatccggtgatcgatgtgcgtcgcaatattgt |
| | | | tacctttacgtacaccctcgacgcctgtggtacctacgcccgtaaatgga |
| | | | ccttgaaaggcaaacagacgaaagcgaccctggacaaactgacggccact |
| | | | cagacggttgcgttggttgcgattgcgcttggtcagaccaacccgatttc |
| | | | cgctggcattagccgtgttacacaagaaaatggtgcactgcaatgcgaac |
| | | | cgctggaccggttcaccctgccggatgacctgttgaaggacatcagcgca |
| | | | tatcgcattgcttgggacagaaacgaagaggaactacgcgctcgcagcgt |
| | | | cgaggccctgccggaggcgcaacaagcggaggtccgtgcgctggacgggg |
| | | | tgtccaaagaaaccgcgcgtacccaactgtgcgcagatttcggcctggac |
| | | | ccgaaacgtctgccgtgggataaaatgagctccaatactacctttatcag |
| | | | cgaagccttgctgtctaacagcgtgagccgtgatcaggtttttttcactc |
| | | | cggctccgaagaagggtgctaagaaaaaagcgcctgttgaagttatgcgt |
| | | | aaagaccgcacttgggcacgtgcgtacaagccgcgtctgagcgtcgaagc |
| | | | tcagaaactgaagaacgaagccctgtgggcacttaagcgcacctccccgg |
| | | | agtatttgaagctgagccgtcgcaaagaggagctgtgtcgtcgtagcatc |
| | | | aactatgttatcgaaaagacccgtcgccgtacgcaatgtcagattgtgat |
| | | | cccggtgatcgaggatctgaatgttcgcttctttcatggttctggtaaga |
| | | | gactgcccggttgggataatttcttcaccgctaaaaaagagaaccgttgg |
| | | | ttcatccagggcctgcacaaagcgtttagcgatctgcgtacgcaccgttc |
| | | | cttctatgtttttgaagttcgtccggagcgcacctctattacctgtccaa |
| | | | agtgcggtcattgcgaggtgggtaatcgtgacggtgaggcgtttcagtgc |
| | | | ctgagctgcggcaagacgtgcaacgcagatcttgacgtcgcgacccataa |
| | | | tctgacccaagtggcgttgaccggtaagaccatgccgaagcgcgaggagc |
| | | | cgcgtgacgctcagggtacagcgccagcgcgcaagactaagaaggcgagc |
| | | | aaatcaaaggcgccaccggcggagcgcgaagaccagaccccggcgcaaga |
| | | | gccaagccagaccagttctggaggtagcgggggtagcggtggtagcacta |
| | | | acttaagtgatattatcgagaaagaaaccgggaaacagctggtgatccaa |
| | | | gaaagcattctgatgctgcccgaagaagttgaagaagtgattggtaacaa |
| | | | gcctgagtcggacatcttagtgcacaccgcgtacgatgaaagcacggatg |
| | | | agaatgtgatgctgctcacctctgatgcccccgaatacaagccgtgggct |
| | | | ctggtgatccaggactctaacggagaaaacaaaattaagatgctctctgg |
| | | | cggctccatggcgcctaaaaaaaaacgcaaagtggggattcacggggttc |
| | | | cggcgtaa |
| 39 | Base editor | mTadA8e-dLbCas12a | atgggaagtcatcaccaccaccaccattcaggtgagaacctgtacttcca |
| | | | gagcatggctccgaaaaagaaacgtaaggtgggtatccacggcgttccgg |
| | | | cgagcggattagtaatgcaaaattataggctaatagacgctacgttgtat |
| | | | gtgacctttgaaccgtgcgttatgtgcgctggtgcaatgattcattctcg |
| | | | tatcggccgtgtcgtgttcggcgtgagaaacagcaagcgcggtgcggcgg |
| | | | gtagcctgatgaatgttctgaactacccgggtatgaaccaccgtgttgaa |
| | | | attaccgagggtatcttggcagacgagtgcgccgccctgctgtgtgattt |
| | | | ctaccgcatgccgcgtcaggtttttaacgcgcagaaaaaggcgcaaagca |
| | | | gcatcaatagcggcggtagctctggcggctcctccggttcggagtctgga |
| | | | agtgagacacccgggacttcagaatccgcgaccccggagtctggaatgtc |
| | | | aaaactagaaaagtttacaaattgttactccctgtcgaaaaccctgagat |
| | | | tcaaagcgatcccggtgggtaaaactcaagagaacattgataacaaacgt |
| | | | ctgctggttgaggacgaaaagcgcgcggaggattacaaaggcgttaaaaa |
| | | | gctgttggaccgttattacctgtcctttattaatgatgttctgcacagca |
| | | | ttaaattaaagaacctaaacaattacatctccctcttccgtaaaaagacc |
| | | | cgcaccgaaaaggaaaataaagaactggagaaccttgagattaatctgcg |
| | | | taaagaaatcgctaaagcgtttaaaggcaacgagggttataaatccttat |
| | | | ttaaaaaagacatcatcgaaacgattctcccagaattcctggacgacaaa |
| | | | gacgagatcgcgctggttaattccttcaacggctttaccaccgcattcac |
| | | | cggcttttttgataaccgcgaaaacatgttctctgaagaggccaaaagca |
| | | | cctcaattgcgtttcgctgcattaacgaaaatctgacgcgttatatctct |
| | | | aacatggatatcttcgagaaggttgacgctatctttgataaacatgaagt |
| | | | ccaggagatcaaggagaagatcctgaacagcgactacgacgtggaggatt |
| | | | tcttcgagggtgagttcttcaattttgttttgacccaagaaggcatcgat |
| | | | gtctacaatgcgataattggtggttttgttaccgagagcggggagaaaat |
| | | | taagggtctgaacgaatatatcaacttgtacaaccagaaaaccaaacaaa |
| | | | aactgccgaagtttaagccgctgtacaagcaagtcttgtcggatcgtgaa |
| | | | tcgctgagcttttacggcgaaggctacaccagcgatgaggaggtgttgga |
| | | | ggtgttccgcaatacgttgaataagaactctgaaattttttccagcatta |
| | | | agaagctggaaaaactattcaagaacttcgacgagtatagcagcgcaggt |
| | | | atctttgtgaaaaacggtccggctattagcaccatctcgaaagacatttt |
| | | | cggcgaatggaatgttattcgtgataagtggaatgccgaatacgacgaca |
| | | | ttcatctgaagaaaaaggcggttgtaactgaaaagtacgaggacgatcgt |
| | | | cgtaagtcgttcaaaaagatcggctccttctctctggagcaactgcaaga |
| | | | gtacgccgatgccgatttgtcagtcgtggagaagctgaaagagatcatca |
| | | | ttcaaaaagttgacgaaatctataaggtctatggtagcagtgaaaaactg |
| | | | tttgacgcggacttcgtgctggagaaaagcctgaagaaaaatgacgccgt |
| | | | ggtggcaatcatgaaagatcttctggatagcgtcaagtctttcgaaaact |
| | | | acatcaaagcgtttttcggtgaaggcaaggaaacgaatcgtgacgagagc |
| | | | ttctatggtgatttcgtcctagcctatgatatcctgcttaaggtggatca |
| | | | catctatgatgccatccgcaactatgttacccagaaaccgtactcaaagg |
| | | | acaagttcaagttatacttccagaatccgcagtttatgggtggctgggat |
| | | | aaagataaggaaaccgactatcgcgccaccattttacgttacggtagtaa |
| | | | gtactatctggcaatcatggataaaaaatatgcgaaatgcctgcaaaaga |
| | | | tcgacaaggacgatgttaacggcaattatgagaagatcaactacaaactg |
| | | | ctgccgggcccaaataagatgctgccgaaggtgtttttcagcaaaaaatg |
| | | | gatggcgtattacaatccgagcgaggacatccaaaagatctacaaaaacg |
| | | | gtacgtttaagaagggtgatatgttcaacctcaacgattgtcataaactt |
| | | | attgacttcttcaaggatagcatatctcgttatccgaagtggagcaacgc |
| | | | gtatgactttaactttagcgaaaccgagaaatacaaagatatcgcgggtt |
| | | | tctatcgtgaagttgaagaacagggttacaaagtgtccttcgagtccgcg |
| | | | agcaagaaggaggtagacaagctggtcgaggagggtaagttgtacatgtt |
| | | | tcagatttacaataaggacttttccgacaagtcccatggtacaccgaatt |
| | | | tgcacaccatgtatttcaagcttctgttcgacgagaacaaccacggccag |
| | | | atccgtctgagcggtggtgcagagctgttcatgcgtagagctagcctgaa |
| | | | gaaagaagagttggtcgttcatccggctaattcgccgatcgcgaacaaaa |
| | | | atccagataaccctaaaaagactacgaccctgagctatgatgtgtataaa |
| | | | gacaaacgctttagcgaagaccagtatgagttacacattccgatcgctat |
| | | | caacaagtgcccgaagaatattttcaagatcaacaccgaagtgcgtgttc |
| | | | ttttgaagcacgatgacaacccgtatgtgattggtattgctcgtggtgaa |
| | | | cgtaatctgctgtacattgtggttgtggacggcaaaggaaacatcgtgga |
| | | | acagtacagcctgaacgaaatcattaacaatttcaacggcattcgcatta |
| | | | agacggattaccattctttgctggacaaaaaggagaaagagcgtttcgag |
| | | | gcccgtcagaactggacctccatcgagaacattaaggagctgaaggcggg |
| | | | ttacatcagccaggtggtgcacaaaatctgcgaactggtggagaagtacg |
| | | | acgcggtgattgctttggcggatttgaactccggttttaagaacagccgc |
| | | | gttaaagtggaaaagcaggtttatcagaaattcgaaaaaatgctgattga |
| | | | taaattgaattacatggttgacaaaaagtctaacccgtgtgcaaccggcg |
| | | | gtgcgctgaagggctaccaaatcaccaataagtttgaaagctttaagagc |
| | | | atgagcacgcaaaatggcttcattttttacattccggcgtggctgaccag |
| | | | caagatcgatccgtcaaccggctttgttaatttactgaagacgaagtata |
| | | | cctctattgccgactctaaaaaatttattagttccttcgatagaatcatg |
| | | | tatgttccggaagaggacctgttcgaatttgcactggactacaagaactt |
| | | | tagccgtaccgatgctgattatataaagaaatggaaactctacagctatg |
| | | | gcaaccgcatccgtatctttcgcaacccgaaaaaaaacaacgtgttcgac |
| | | | tgggaagaggtatgtctgacttcagcgtacaaggagcttttcaataaata |
| | | | tggtatcaactaccagcagggtgatatccgtgcactgctttgcgagcaaa |
| | | | gcgataaagcgttctattcaagcttcatggcgctgatgagcctgatgctg |
| | | | caaatgcgcaacagcatcaccggtcgtaccgatgttgcatttctgatttc |
| | | | tccagtaaaaaacagcgatggtatcttttacgacagccgtaattacgagg |
| | | | cccaagaaaatgcgattttgccgaagaacgctgacgcgaatggtgcatac |
| | | | aacattgcaaggaaagttctgtgggcaatcggccaatttaaaaaagcgga |
| | | | agatgagaagctggacaaagtgaaaattgcgattagcaacaaggagtggc |
| | | | tggagtatgcgcagacctccgttaaatcagggggaagtggcggaagcggt |
| | | | gggtctacgaacctgagcgacattattgaaaaagaaactggtaaacagtt |
| | | | ggtgatccaagaatccattctgatgttaccggaggaagtggaagaggtga |
| | | | tcggcaacaagccggagtccgacatcctggttcacaccgcgtatgatgag |
| | | | tcgaccgatgagaatgttatgctgctgaccagcgacgccccagagtacaa |
| | | | accgtgggcgctcgtcatccaggatagcaatggcgaaaacaagatcaaga |
| | | | tgttgtctggcggtagcatggctccgaaaaagaagcgtaaagtaggtatt |
| | | | catggtgttccggcatcaggttaa |
| 40 | Base editor | pmCDA1-dCas9 | atgggaagtcatcaccaccaccaccattcaggtgagaacctgtacttcca |
| | | | gagcatggctccgaaaaagaaacgtaaggtgggtatccacggcgttccgg |
| | | | cgatgacagatgctgaatatgtaaggatacacgagaagttggacatctac |
| | | | acctttaaaaagcagttctttaacaacaaaaagagcgtcagccatcgttg |
| | | | ctatgttctgtttgagttgaagcgccgtggcgaacgtcgtgcgtgctttt |
| | | | ggggttatgctgtgaacaaaccgcagtctggtactgaacgcggtattcat |
| | | | gcagaaattttcagcattcgtaaggtggaagaatacctgcgcgataaccc |
| | | | aggtcagttcaccattaactggtacagcagctggtccccgtgcgcggatt |
| | | | gcgctgagaagatcttggagtggtacaatcaagagctgcgtggtaatggc |
| | | | cataccctgaaaatctgggcgtgcaaactgtattacgaaaagaacgcgcg |
| | | | taatcaaattggtctgtggaatctgcgtgacaacggcgtgggcctgaatg |
| | | | ttatggtttccgagcactatcagtgttgtcgtaaaatcttcatccaatcc |
| | | | tctcacaaccaattgaacgagaaccgctggctggagaagaccctgaagcg |
| | | | cgccgagaaaagacgtagcgaactctcgatcatgattcaggttaaaatct |
| | | | tacacacgaccaaaagcccggcagtgtctggaagtgagacacccgggact |
| | | | tcagaatccgcgaccccggagtctggaatggataaaaagtattcaatagg |
| | | | actagctattggcaccaacagcgttggttgggcagttatcaccgatgaat |
| | | | ataaggtcccgagcaagaagtttaaggtcctgggcaacaccgatcgccac |
| | | | agcataaaaaagaaccttattggtgcgctgctgttcgattctggtgagac |
| | | | tgccgaagcgacccgtctgaagagaaccgcgcgtcgtagatacacccggc |
| | | | gcaagaaccgtatctgctatctgcaagaaattttcagcaacgagatggct |
| | | | aaggtcgacgactccttctttcatcgcctggaggaaagctttctggttga |
| | | | ggaagacaaaaagcacgagcgtcatccgattttcggtaacattgttgatg |
| | | | aagttgcctaccacgaaaaataccccacaatttatcatctgcgtaaaaaa |
| | | | ctggtagactccaccgataaggcggatttacgccttatctatttggcgct |
| | | | ggcgcatatgattaagttccgcggtcattttttgatcgagggcgacctga |
| | | | atccggataacagcgacgttgacaaactgtttatccagctggtgcagacc |
| | | | tataaccagttgttcgaagagaacccgattaatgcgtctggcgtggacgc |
| | | | gaaggcgatcctgagcgctcgtctgtccaagtcacgccgcctggagaact |
| | | | tgatcgcgcaactgccgggtgagaagaaaaacggcttgttcggcaatctg |
| | | | attgctctgtccctaggtttgaccccgaatttcaaatctaattttgatct |
| | | | ggcggaagatgcgaaattacagttgagcaaggacacttacgatgacgatc |
| | | | tggacaacctgctcgctcagatcggcgatcagtatgccgatcttttcctg |
| | | | gccgcgaagaacctgagcgatgccatcttgctatcggacatcttacgtgt |
| | | | taacaccgaaatcaccaaagctccgctaagcgcgtccatgattaaacgtt |
| | | | acgatgaacaccaccaggatctgacgctgctgaaagcgttggtgcgccag |
| | | | caactgccggaaaaatacaaggaaattttcttcgaccaaagcaagaacgg |
| | | | ttatgcaggctatatcgacggtggtgcgtcgcaagaagaattttataaat |
| | | | tcatcaaaccgattttggagaagatggatggtaccgaggagctgctggta |
| | | | aaattgaatcgggaagacctcttgcgaaagcagcgtactttcgataatgg |
| | | | tagcatcccgcaccagattcatctgggcgaacttcacgcaatcctacgtc |
| | | | gtcaagaggatttctacccgtttctgaaggataaccgtgaaaagatcgag |
| | | | aagattctgacctttcgtattccgtattatgtgggtccgctggcgagagg |
| | | | caacagccgttttgcgtggatgacccgcaagtcggaagagactatcacgc |
| | | | cgtggaatttcgaggaggtcgtggacaagggtgcatcagcgcaatctttt |
| | | | attgaacgtatgaccaattttgataaaaaccttccaaatgaaaaagtgtt |
| | | | gccgaagcacagcttgctgtacgagtatttcaccgtttacaatgaactga |
| | | | cgaaagtcaagtacgttaccgaaggtatgcgtaagccagcctttctgtct |
| | | | ggggagcaaaaaaaagccattgtggatctgctgttcaagaccaatcgcaa |
| | | | ggttaccgtcaagcaactgaaagaggattacttcaaaaaaatcgaatgtt |
| | | | ttgactccgtggaaatcagcggcgttgaagaccgctttaatgcatctctg |
| | | | ggcacctaccacgatctactcaagatcatcaaggataaggacttcctgga |
| | | | caacgaagaaaatgaggatatcttggaagatattgtgctgactttaacgt |
| | | | tgttcgaggatcgtgagatgattgaagaacgcctcaaaacgtacgcacat |
| | | | ctgtttgacgataaggtgatgaaacagctaaagcgccgtcgttacaccgg |
| | | | ttggggtcgtctgtctagaaagctgattaacggcattcgtgacaaacaat |
| | | | ctggcaagaccatcctggacttcctcaaatccgacggcttcgcaaatcgt |
| | | | aatttcatgcaactgatccatgatgatagcctgacctttaaagaagatat |
| | | | tcagaaggctcaagtgtccggccagggtgactcgctgcatgagcatatcg |
| | | | cgaatctggctggctcccctgcgataaaaaaaggtattctccaaacggtg |
| | | | aaagttgtggatgaactggttaaagtcatgggtcgtcataaaccggaaaa |
| | | | catcgtgattgagatggcacgtgagaaccagaccacccagaaaggtcaaa |
| | | | aaaacagtcgtgagcgtatgaaacgtatcgaagaggggattaaggagtta |
| | | | ggcagccagattctgaaggagcacccggttgagaacacccagctgcaaaa |
| | | | cgagaagctctacctgtattacctgcaaaacggccgtgacatgtatgttg |
| | | | accaggagctggacatcaatcgtctcagcgactacgacgtggatgctatc |
| | | | gtgccgcagagctttctaaaagatgattccatcgacaataaagtgctcac |
| | | | ccgtagcgacaagaaccgtggtaaaagcgacaacgtgccgagcgaggaag |
| | | | tagttaagaaaatgaaaaactactggcgtcagctattgaacgccaagctt |
| | | | atcacccagcgtaagtttgacaacttgacgaaggcagagcgtggcggcct |
| | | | gagcgagctagacaaggcgggttttattaaacgccagctggtggaaactc |
| | | | gccagattaccaaacacgttgcgcaaatcttggactcacgcatgaatacg |
| | | | aagtacgacgaaaacgataaattgatccgcgaggtgaaggtcataaccct |
| | | | gaaatctaaactggttagcgatttccggaaggactttcaattttataaag |
| | | | tccgggagattaacaactatcaccatgcacacgacgcttacttaaatgcg |
| | | | gttgttggcacggccctgatcaagaagtacccgaaactggaaagcgagtt |
| | | | tgtttatggtgactacaaggtgtatgacgttcgcaagatgattgcaaaat |
| | | | ccgaacaagagattggtaaggcgacggctaagtattttttctacagcaat |
| | | | atcatgaacttcttcaagactgaaatcaccctggcgaacggtgaaattcg |
| | | | taaacgtccgttgatcgaaaccaacggtgaaaccggtgaaatcgtttggg |
| | | | ataaaggtagggattttgcgaccgttcgtaaggtcctgagcatgccgcag |
| | | | gttaacatcgtgaaaaagaccgaagtgcagaccggcggtttctccaagga |
| | | | gagtatcttaccgaagcgcaacagtgacaagctgatcgcgcgtaaaaaag |
| | | | actgggatccgaaaaaatacggcggctttgactccccgaccgtcgcgtac |
| | | | agcgtactggtcgtggctaaagtggagaaaggcaaatcgaaaaagctgaa |
| | | | atccgttaaagagttgctgggtattacgattatggaacgttcgagcttcg |
| | | | agaaaaatccgatcgacttcctggaggccaaaggctacaaggaagttaaa |
| | | | aaagacctgatcattaaactgccgaagtacagcctgttcgagctcgagaa |
| | | | cggtcgcaaacgcatgctggcgagcgcgggtgagctccaaaaaggcaacg |
| | | | aattggctctgccgagcaagtacgtgaacttcctgtatctggcctcccat |
| | | | tatgaaaaactgaagggtagcccggaagataacgaacaaaagcagctgtt |
| | | | cgtggaacaacataaacactatctggacgagattattgagcaaatcagcg |
| | | | agttcagcaaacgcgtgattctggcggatgctaatctggacaaggttctt |
| | | | tcagcctataataaacaccgtgataagccgatccgtgaacaggcagaaaa |
| | | | tattattcacctgttcaccctgacgaacctgggtgcgccagcagcgttta |
| | | | aatacttcgacacaacgatcgaccggaagcgttataccagcaccaaagag |
| | | | gttcttgacgccaccttgattcaccaaagcatcacgggtctgtatgaaac |
| | | | ccgtattgacttgagccagttgggcggagattcagggggaagtggcggaa |
| | | | gcggtgggtctacgaacctgagcgacattattgaaaaagaaactggtaaa |
| | | | cagttggtgatccaagaatccattctgatgttaccggaggaagtggaaga |
| | | | ggtgatcggcaacaagccggagtccgacatcctggttcacaccgcgtatg |
| | | | atgagtcgaccgatgagaatgttatgctgctgaccagcgacgccccagag |
| | | | tacaaaccgtgggcgctcgtcatccaggatagcaatggcgaaaacaagat |
| | | | caagatgttgtctggcggtagcatggctccgaaaaagaagcgtaaagtag |
| | | | gtattcatggtgttccggcatcaggttaa |
| 41 | Base editor | pmCDA1-dCasPhi-2 | atgggaagtcatcaccaccaccaccattcaggtgagaacctgtacttcca |
| | | | gagcatggctccgaaaaagaaacgtaaggtgggtatccacggcgttccgg |
| | | | cgatgacagatgctgaatatgtaaggatacacgagaagttggacatctac |
| | | | acctttaaaaagcagttctttaacaacaaaaagagcgtcagccatcgttg |
| | | | ctatgttctgtttgagttgaagcgccgtggcgaacgtcgtgcgtgctttt |
| | | | ggggttatgctgtgaacaaaccgcagtctggtactgaacgcggtattcat |
| | | | gcagaaattttcagcattcgtaaggtggaagaatacctgcgcgataaccc |
| | | | aggtcagttcaccattaactggtacagcagctggtccccgtgcgcggatt |
| | | | gcgctgagaagatcttggagtggtacaatcaagagctgcgtggtaatggc |
| | | | cataccctgaaaatctgggcgtgcaaactgtattacgaaaagaacgcgcg |
| | | | taatcaaattggtctgtggaatctgcgtgacaacggcgtgggcctgaatg |
| | | | ttatggtttccgagcactatcagtgttgtcgtaaaatcttcatccaatcc |
| | | | tctcacaaccaattgaacgagaaccgctggctggagaagaccctgaagcg |
| | | | cgccgagaaaagacgtagcgaactctcgatcatgattcaggttaaaatct |
| | | | tacacacgaccaaaagcccggcagtgtctggaagtgagacacccgggact |
| | | | tcagaatccgcgaccccggagtctggaatgccaaaacccgctgtagagtc |
| | | | agaattttcaaaagttttgaaaaagcactttccgggagaacgtttccgct |
| | | | cgagctatatgaaaagaggcggtaagatcctggctgcccaaggcgaagag |
| | | | gcagttgttgcgtatctacagggcaagtccgaggaggagcctccgaactt |
| | | | ccagccaccggcgaagtgccacgtggtgaccaaatcccgtgacttcgcgg |
| | | | aatggccgatcatgaaggcgtcggaagcgattcaacgttatatctatgca |
| | | | ctgtcgaccaccgaacgcgcggcctgtaaaccgggcaaaagtagtgagag |
| | | | ccatgcagcgtggttcgccgctaccggtgtcagcaaccacggctacagcc |
| | | | acgttcagggtctgaacctgatttttgatcataccttgggccgttacgat |
| | | | ggtgttctgaaaaaggtccaactgcgcaacgaaaaggcgagagcgcgttt |
| | | | agagagcattaacgcgtctagagcagatgaaggtttaccggagattaaag |
| | | | ccgaggaggaagaagtggctacgaacgaaaccggccacctgttgcaaccg |
| | | | ccgggtatcaatccgagcttttacgtgtatcagaccattagcccgcaggc |
| | | | ataccgtccgcgtgacgagatcgtgctgccgccggagtacgcaggttacg |
| | | | tacgtgacccgaatgctccgattccgctgggtgtggtgcgtaaccgttgc |
| | | | gatatccaaaaagggtgcccgggctacatcccggaatggcagcgtgaagc |
| | | | aggtaccgcgatcagcccgaaaacaggcaaggccgtaactgtgccgggcc |
| | | | tgtctccgaagaaaaacaaacgtatgcgtaggtactggcgctctgagaag |
| | | | gagaaagctcaagatgcgctgttggttaccgtacgcattggtaccgattg |
| | | | ggttgtcatcgacgtgcgcggtctgttgcgcaacgcccgttggcgcacca |
| | | | tcgctcccaaggacatcagcctgaatgcgctcctcgatttgtttaccggt |
| | | | gatccggtgatcgatgtgcgtcgcaatattgttacctttacgtacaccct |
| | | | cgacgcctgtggtacctacgcccgtaaatggaccttgaaaggcaaacaga |
| | | | cgaaagcgaccctggacaaactgacggccactcagacggttgcgttggtt |
| | | | gcgattgcgcttggtcagaccaacccgatttccgctggcattagccgtgt |
| | | | tacacaagaaaatggtgcactgcaatgcgaaccgctggaccggttcaccc |
| | | | tgccggatgacctgttgaaggacatcagcgcatatcgcattgcttgggac |
| | | | agaaacgaagaggaactacgcgctcgcagcgtcgaggccctgccggaggc |
| | | | gcaacaagcggaggtccgtgcgctggacggggtgtccaaagaaaccgcgc |
| | | | gtacccaactgtgcgcagatttcggcctggacccgaaacgtctgccgtgg |
| | | | gataaaatgagctccaatactacctttatcagcgaagccttgctgtctaa |
| | | | cagcgtgagccgtgatcaggtttttttcactccggctccgaagaagggtg |
| | | | ctaagaaaaaagcgcctgttgaagttatgcgtaaagaccgcacttgggca |
| | | | cgtgcgtacaagccgcgtctgagcgtcgaagctcagaaactgaagaacga |
| | | | agccctgtgggcacttaagcgcacctccccggagtatttgaagctgagcc |
| | | | gtcgcaaagaggagctgtgtcgtcgtagcatcaactatgttatcgaaaag |
| | | | acccgtcgccgtacgcaatgtcagattgtgatcccggtgatcgaggatct |
| | | | gaatgttcgcttctttcatggttctggtaagagactgcccggttgggata |
| | | | atttcttcaccgctaaaaaagagaaccgttggttcatccagggcctgcac |
| | | | aaagcgtttagcgatctgcgtacgcaccgttccttctatgtttttgaagt |
| | | | tcgtccggagcgcacctctattacctgtccaaagtgcggtcattgcgagg |
| | | | tgggtaatcgtgacggtgaggcgtttcagtgcctgagctgcggcaagacg |
| | | | tgcaacgcagatcttgacgtcgcgacccataatctgacccaagtggcgtt |
| | | | gaccggtaagaccatgccgaagcgcgaggagccgcgtgacgctcagggta |
| | | | cagcgccagcgcgcaagactaagaaggcgagcaaatcaaaggcgccaccg |
| | | | gcggagcgcgaagaccagaccccggcgcaagagccaagccagaccagttc |
| | | | tggaggtagcgggggtagcggtggtagcactaacttaagtgatattatcg |
| | | | agaaagaaaccgggaaacagctggtgatccaagaaagcattctgatgctg |
| | | | cccgaagaagttgaagaagtgattggtaacaagcctgagtcggacatctt |
| | | | agtgcacaccgcgtacgatgaaagcacggatgagaatgtgatgctgctca |
| | | | cctctgatgcccccgaatacaagccgtgggctctggtgatccaggactct |
| | | | aacggagaaaacaaaattaagatgctctctggcggctccatggcgcctaa |
| | | | aaaaaaacgcaaagtggggattcacggggttccggcgtaa |
| 42 | Base editor | pmCDA1-dLbCas12a | atgggaagtcatcaccaccaccaccattcaggtgagaacctgtacttccag |
| | | | agcatggctccgaaaaagaaacgtaaggtgggtatccacggcgttccggc |
| | | | gatgacagatgctgaatatgtaaggatacacgagaagttggacatctaca |
| | | | cctttaaaaagcagttctttaacaacaaaaagagcgtcagccatcgttgc |
| | | | tatgttctgtttgagttgaagcgccgtggcgaacgtcgtgcgtgcttttg |
| | | | gggttatgctgtgaacaaaccgcagtctggtactgaacgcggtattcatg |
| | | | cagaaattttcagcattcgtaaggtggaagaatacctgcgcgataaccca |
| | | | ggtcagttcaccattaactggtacagcagctggtccccgtgcgcggattg |
| | | | cgctgagaagatcttggagtggtacaatcaagagctgcgtggtaatggcc |
| | | | ataccctgaaaatctgggcgtgcaaactgtattacgaaaagaacgcgcgt |
| | | | aatcaaattggtctgtggaatctgcgtgacaacggcgtgggcctgaatgt |
| | | | tatggtttccgagcactatcagtgttgtcgtaaaatcttcatccaatcct |
| | | | ctcacaaccaattgaacgagaaccgctggctggagaagaccctgaagcgc |
| | | | gccgagaaaagacgtagcgaactctcgatcatgattcaggttaaaatctt |
| | | | acacacgaccaaaagcccggcagtgtctggaagtgagacacccgggactt |
| | | | cagaatccgcgaccccggagtctggaatgtcaaaactagaaaagtttaca |
| | | | aattgttactccctgtcgaaaaccctgagattcaaagcgatcccggtggg |
| | | | taaaactcaagagaacattgataacaaacgtctgctggttgaggacgaaa |
| | | | agcgcgcggaggattacaaaggcgttaaaaagctgttggaccgttattac |
| | | | ctgtcctttattaatgatgttctgcacagcattaaattaaagaacctaaa |
| | | | caattacatctccctcttccgtaaaaagacccgcaccgaaaaggaaaata |
| | | | aagaactggagaaccttgagattaatctgcgtaaagaaatcgctaaagcg |
| | | | tttaaaggcaacgagggttataaatccttatttaaaaaagacatcatcga |
| | | | aacgattctcccagaattcctggacgacaaagacgagatcgcgctggtta |
| | | | attccttcaacggctttaccaccgcattcaccggcttttttgataaccgc |
| | | | gaaaacatgttctctgaagaggccaaaagcacctcaattgcgtttcgctg |
| | | | cattaacgaaaatctgacgcgttatatctctaacatggatatcttcgaga |
| | | | aggttgacgctatctttgataaacatgaagtccaggagatcaaggagaag |
| | | | atcctgaacagcgactacgacgtggaggatttcttcgagggtgagttctt |
| | | | caattttgttttgacccaagaaggcatcgatgtctacaatgcgataattg |
| | | | gtggttttgttaccgagagcggggagaaaattaagggtctgaacgaatat |
| | | | atcaacttgtacaaccagaaaaccaaacaaaaactgccgaagtttaagcc |
| | | | gctgtacaagcaagtcttgtcggatcgtgaatcgctgagcttttacggcg |
| | | | aaggctacaccagcgatgaggaggtgttggaggtgttccgcaatacgttg |
| | | | aataagaactctgaaattttttccagcattaagaagctggaaaaactatt |
| | | | caagaacttcgacgagtatagcagcgcaggtatctttgtgaaaaacggtc |
| | | | cggctattagcaccatctcgaaagacattttcggcgaatggaatgttatt |
| | | | cgtgataagtggaatgccgaatacgacgacattcatctgaagaaaaaggc |
| | | | ggttgtaactgaaaagtacgaggacgatcgtcgtaagtcgttcaaaaaga |
| | | | tcggctccttctctctggagcaactgcaagagtacgccgatgccgatttg |
| | | | tcagtcgtggagaagctgaaagagatcatcattcaaaaagttgacgaaat |
| | | | ctataaggtctatggtagcagtgaaaaactgtttgacgcggacttcgtgc |
| | | | tggagaaaagcctgaagaaaaatgacgccgtggtggcaatcatgaaagat |
| | | | cttctggatagcgtcaagtctttcgaaaactacatcaaagcgtttttcgg |
| | | | tgaaggcaaggaaacgaatcgtgacgagagcttctatggtgatttcgtcc |
| | | | tagcctatgatatcctgcttaaggtggatcacatctatgatgccatccgc |
| | | | aactatgttacccagaaaccgtactcaaaggacaagttcaagttatactt |
| | | | ccagaatccgcagtttatgggtggctgggataaagataaggaaaccgact |
| | | | atcgcgccaccattttacgttacggtagtaagtactatctggcaatcatg |
| | | | gataaaaaatatgcgaaatgcctgcaaaagatcgacaaggacgatgttaa |
| | | | cggcaattatgagaagatcaactacaaactgctgccgggcccaaataaga |
| | | | tgctgccgaaggtgtttttcagcaaaaaatggatggcgtattacaatccg |
| | | | agcgaggacatccaaaagatctacaaaaacggtacgtttaagaagggtga |
| | | | tatgttcaacctcaacgattgtcataaacttattgacttcttcaaggata |
| | | | gcatatctcgttatccgaagtggagcaacgcgtatgactttaactttagc |
| | | | gaaaccgagaaatacaaagatatcgcgggtttctatcgtgaagttgaaga |
| | | | acagggttacaaagtgtccttcgagtccgcgagcaagaaggaggtagaca |
| | | | agctggtcgaggagggtaagttgtacatgtttcagatttacaataaggac |
| | | | ttttccgacaagtcccatggtacaccgaatttgcacaccatgtatttcaa |
| | | | gcttctgttcgacgagaacaaccacggccagatccgtctgagcggtggtg |
| | | | cagagctgttcatgcgtagagctagcctgaagaaagaagagttggtcgtt |
| | | | catccggctaattcgccgatcgcgaacaaaaatccagataaccctaaaaa |
| | | | gactacgaccctgagctatgatgtgtataaagacaaacgctttagcgaag |
| | | | accagtatgagttacacattccgatcgctatcaacaagtgcccgaagaat |
| | | | attttcaagatcaacaccgaagtgcgtgttcttttgaagcacgatgacaa |
| | | | cccgtatgtgattggtattgctcgtggtgaacgtaatctgctgtacattg |
| | | | tggttgtggacggcaaaggaaacatcgtggaacagtacagcctgaacgaa |
| | | | atcattaacaatttcaacggcattcgcattaagacggattaccattcttt |
| | | | gctggacaaaaaggagaaagagcgtttcgaggcccgtcagaactggacct |
| | | | ccatcgagaacattaaggagctgaaggcgggttacatcagccaggtggtg |
| | | | cacaaaatctgcgaactggtggagaagtacgacgcggtgattgctttggc |
| | | | ggatttgaactccggttttaagaacagccgcgttaaagtggaaaagcagg |
| | | | tttatcagaaattcgaaaaaatgctgattgataaattgaattacatggtt |
| | | | gacaaaaagtctaacccgtgtgcaaccggcggtgcgctgaagggctacca |
| | | | aatcaccaataagtttgaaagctttaagagcatgagcacgcaaaatggct |
| | | | tcattttttacattccggcgtggctgaccagcaagatcgatccgtcaacc |
| | | | ggctttgttaatttactgaagacgaagtatacctctattgccgactctaa |
| | | | aaaatttattagttccttcgatagaatcatgtatgttccggaagaggacc |
| | | | tgttcgaatttgcactggactacaagaactttagccgtaccgatgctgat |
| | | | tatataaagaaatggaaactctacagctatggcaaccgcatccgtatctt |
| | | | tcgcaacccgaaaaaaaacaacgtgttcgactgggaagaggtatgtctga |
| | | | cttcagcgtacaaggagcttttcaataaatatggtatcaactaccagcag |
| | | | ggtgatatccgtgcactgctttgcgagcaaagcgataaagcgttctattc |
| | | | aagcttcatggcgctgatgagcctgatgctgcaaatgcgcaacagcatca |
| | | | ccggtcgtaccgatgttgcatttctgatttctccagtaaaaaacagcgat |
| | | | ggtatcttttacgacagccgtaattacgaggcccaagaaaatgcgatttt |
| | | | gccgaagaacgctgacgcgaatggtgcatacaacattgcaaggaaagttc |
| | | | tgtgggcaatcggccaatttaaaaaagcggaagatgagaagctggacaaa |
| | | | gtgaaaattgcgattagcaacaaggagtggctggagtatgcgcagacctc |
| | | | cgttaaatcagggggaagtggcggaagcggtgggtctacgaacctgagcg |
| | | | acattattgaaaaagaaactggtaaacagttggtgatccaagaatccatt |
| | | | ctgatgttaccggaggaagtggaagaggtgatcggcaacaagccggagtc |
| | | | cgacatcctggttcacaccgcgtatgatgagtcgaccgatgagaatgtta |
| | | | tgctgctgaccagcgacgccccagagtacaaaccgtgggcgctcgtcatc |
| | | | caggatagcaatggcgaaaacaagatcaagatgttgtctggcggtagcat |
| | | | ggctccgaaaaagaagcgtaaagtaggtattcatggtgttccggcatcag |
| | | | gttaa |
| 43 | Base editor | PpA1-R33A-dCas9 | atgggaagtcatcaccaccaccaccattcaggtgagaacctgtacttcca |
| | | | gagcatggctccgaaaaagaaacgtaaggtgggtatccacggcgttccgg |
| | | | cgatgacaagtgaaaaaggaccctcaactggggacccaaccctgcgtcgt |
| | | | cgcattgaaagctgggagttcgacgttttttatgatccgcgtgagctggc |
| | | | gaaagagacgtgcctgttgtacgagatcaaatggggtatgagccgtaaaa |
| | | | tctggagatccagcggtaaaaacaccaccaatcacgtggaagtgaatttt |
| | | | atcaagaagttcaccagcgaacgtcgtttccacagctccatttcttgttc |
| | | | tattacctggtttctgtcttggtcgccgtgttgggagtgctcccaggcaa |
| | | | ttcgtgaattccttagtcaacatccgggcgtgaccctggtcatctacgtc |
| | | | gcccgtttgttttggcatatggaccagcgcaatcgtcagggtttgcgcga |
| | | | tctggttaacagcggcgttacgatccaaattatgcgtgcgagcgagtatt |
| | | | accactgctggcgcaacttcgtgaactatccaccgggcgatgaagcacat |
| | | | tggccgcaatatccgccgctgtggatgatgctgtacgcgctggaactcca |
| | | | ctgcattatcctgtcgctgccgccttgtctgaagatcagccgacgctggc |
| | | | agaaccacctcgcgtttttccgcctgcacttgcaaaactgccattaccag |
| | | | accatcccgccgcatatcctgttagctaccggtctgattcacccgagcgt |
| | | | tacttggcgttctggaagtgagacacccgggacttcagaatccgcgaccc |
| | | | cggagtctggaatggataaaaagtattcaataggactagctattggcacc |
| | | | aacagcgttggttgggcagttatcaccgatgaatataaggtcccgagcaa |
| | | | gaagtttaaggtcctgggcaacaccgatcgccacagcataaaaaagaacc |
| | | | ttattggtgcgctgctgttcgattctggtgagactgccgaagcgacccgt |
| | | | ctgaagagaaccgcgcgtcgtagatacacccggcgcaagaaccgtatctg |
| | | | ctatctgcaagaaattttcagcaacgagatggctaaggtcgacgactcct |
| | | | tctttcatcgcctggaggaaagctttctggttgaggaagacaaaaagcac |
| | | | gagcgtcatccgattttcggtaacattgttgatgaagttgcctaccacga |
| | | | aaaataccccacaatttatcatctgcgtaaaaaactggtagactccaccg |
| | | | ataaggcggatttacgccttatctatttggcgctggcgcatatgattaag |
| | | | ttccgcggtcattttttgatcgagggcgacctgaatccggataacagcga |
| | | | cgttgacaaactgtttatccagctggtgcagacctataaccagttgttcg |
| | | | aagagaacccgattaatgcgtctggcgtggacgcgaaggcgatcctgagc |
| | | | gctcgtctgtccaagtcacgccgcctggagaacttgatcgcgcaactgcc |
| | | | gggtgagaagaaaaacggcttgttcggcaatctgattgctctgtccctag |
| | | | gtttgaccccgaatttcaaatctaattttgatctggcggaagatgcgaaa |
| | | | ttacagttgagcaaggacacttacgatgacgatctggacaacctgctcgc |
| | | | tcagatcggcgatcagtatgccgatcttttcctggccgcgaagaacctga |
| | | | gcgatgccatcttgctatcggacatcttacgtgttaacaccgaaatcacc |
| | | | aaagctccgctaagcgcgtccatgattaaacgttacgatgaacaccacca |
| | | | ggatctgacgctgctgaaagcgttggtgcgccagcaactgccggaaaaat |
| | | | acaaggaaattttcttcgaccaaagcaagaacggttatgcaggctatatc |
| | | | gacggtggtgcgtcgcaagaagaattttataaattcatcaaaccgatttt |
| | | | ggagaagatggatggtaccgaggagctgctggtaaaattgaatcgggaag |
| | | | acctcttgcgaaagcagcgtactttcgataatggtagcatcccgcaccag |
| | | | attcatctgggcgaacttcacgcaatcctacgtcgtcaagaggatttcta |
| | | | cccgtttctgaaggataaccgtgaaaagatcgagaagattctgacctttc |
| | | | gtattccgtattatgtgggtccgctggcgagaggcaacagccgttttgcg |
| | | | tggatgacccgcaagtcggaagagactatcacgccgtggaatttcgagga |
| | | | ggtcgtggacaagggtgcatcagcgcaatcttttattgaacgtatgacca |
| | | | attttgataaaaaccttccaaatgaaaaagtgttgccgaagcacagcttg |
| | | | ctgtacgagtatttcaccgtttacaatgaactgacgaaagtcaagtacgt |
| | | | taccgaaggtatgcgtaagccagcctttctgtctggggagcaaaaaaaag |
| | | | ccattgtggatctgctgttcaagaccaatcgcaaggttaccgtcaagcaa |
| | | | ctgaaagaggattacttcaaaaaaatcgaatgttttgactccgtggaaat |
| | | | cagcggcgttgaagaccgctttaatgcatctctgggcacctaccacgatc |
| | | | tactcaagatcatcaaggataaggacttcctggacaacgaagaaaatgag |
| | | | gatatcttggaagatattgtgctgactttaacgttgttcgaggatcgtga |
| | | | gatgattgaagaacgcctcaaaacgtacgcacatctgtttgacgataagg |
| | | | tgatgaaacagctaaagcgccgtcgttacaccggttggggtcgtctgtct |
| | | | agaaagctgattaacggcattcgtgacaaacaatctggcaagaccatcct |
| | | | ggacttcctcaaatccgacggcttcgcaaatcgtaatttcatgcaactga |
| | | | tccatgatgatagcctgacctttaaagaagatattcagaaggctcaagtg |
| | | | tccggccagggtgactcgctgcatgagcatatcgcgaatctggctggctc |
| | | | ccctgcgataaaaaaaggtattctccaaacggtgaaagttgtggatgaac |
| | | | tggttaaagtcatgggtcgtcataaaccggaaaacatcgtgattgagatg |
| | | | gcacgtgagaaccagaccacccagaaaggtcaaaaaaacagtcgtgagcg |
| | | | tatgaaacgtatcgaagaggggattaaggagttaggcagccagattctga |
| | | | aggagcacccggttgagaacacccagctgcaaaacgagaagctctacctg |
| | | | tattacctgcaaaacggccgtgacatgtatgttgaccaggagctggacat |
| | | | caatcgtctcagcgactacgacgtggatgctatcgtgccgcagagctttc |
| | | | taaaagatgattccatcgacaataaagtgctcacccgtagcgacaagaac |
| | | | cgtggtaaaagcgacaacgtgccgagcgaggaagtagttaagaaaatgaa |
| | | | aaactactggcgtcagctattgaacgccaagcttatcacccagcgtaagt |
| | | | ttgacaacttgacgaaggcagagcgtggcggcctgagcgagctagacaag |
| | | | gcgggttttattaaacgccagctggtggaaactcgccagattaccaaaca |
| | | | cgttgcgcaaatcttggactcacgcatgaatacgaagtacgacgaaaacg |
| | | | ataaattgatccgcgaggtgaaggtcataaccctgaaatctaaactggtt |
| | | | agcgatttccggaaggactttcaattttataaagtccgggagattaacaa |
| | | | ctatcaccatgcacacgacgcttacttaaatgcggttgttggcacggccc |
| | | | tgatcaagaagtacccgaaactggaaagcgagtttgtttatggtgactac |
| | | | aaggtgtatgacgttcgcaagatgattgcaaaatccgaacaagagattgg |
| | | | taaggcgacggctaagtattttttctacagcaatatcatgaacttcttca |
| | | | agactgaaatcaccctggcgaacggtgaaattcgtaaacgtccgttgatc |
| | | | gaaaccaacggtgaaaccggtgaaatcgtttgggataaaggtagggattt |
| | | | tgcgaccgttcgtaaggtcctgagcatgccgcaggttaacatcgtgaaaa |
| | | | agaccgaagtgcagaccggcggtttctccaaggagagtatcttaccgaag |
| | | | cgcaacagtgacaagctgatcgcgcgtaaaaaagactgggatccgaaaaa |
| | | | atacggcggctttgactccccgaccgtcgcgtacagcgtactggtcgtgg |
| | | | ctaaagtggagaaaggcaaatcgaaaaagctgaaatccgttaaagagttg |
| | | | ctgggtattacgattatggaacgttcgagcttcgagaaaaatccgatcga |
| | | | cttcctggaggccaaaggctacaaggaagttaaaaaagacctgatcatta |
| | | | aactgccgaagtacagcctgttcgagctcgagaacggtcgcaaacgcatg |
| | | | ctggcgagcgcgggtgagctccaaaaaggcaacgaattggctctgccgag |
| | | | caagtacgtgaacttcctgtatctggcctcccattatgaaaaactgaagg |
| | | | gtagcccggaagataacgaacaaaagcagctgttcgtggaacaacataaa |
| | | | cactatctggacgagattattgagcaaatcagcgagttcagcaaacgcgt |
| | | | gattctggcggatgctaatctggacaaggttctttcagcctataataaac |
| | | | accgtgataagccgatccgtgaacaggcagaaaatattattcacctgttc |
| | | | accctgacgaacctgggtgcgccagcagcgtttaaatacttcgacacaac |
| | | | gatcgaccggaagcgttataccagcaccaaagaggttcttgacgccacct |
| | | | tgattcaccaaagcatcacgggtctgtatgaaacccgtattgacttgagc |
| | | | cagttgggcggagattcagggggaagtggcggaagcggtgggtctacgaa |
| | | | cctgagcgacattattgaaaaagaaactggtaaacagttggtgatccaag |
| | | | aatccattctgatgttaccggaggaagtggaagaggtgatcggcaacaag |
| | | | ccggagtccgacatcctggttcacaccgcgtatgatgagtcgaccgatga |
| | | | gaatgttatgctgctgaccagcgacgccccagagtacaaaccgtgggcgc |
| | | | tcgtcatccaggatagcaatggcgaaaacaagatcaagatgttgtctggc |
| | | | ggtagcatggctccgaaaaagaagcgtaaagtaggtattcatggtgttcc |
| | | | ggcatcaggttaa |
| 44 | Base editor | PpA1-R33A- | atgggaagtcatcaccaccaccaccattcaggtgagaacctgtacttcca |
| | | dCasPhi-2 | gagcatggctccgaaaaagaaacgtaaggtgggtatccacggcgttccgg |
| | | | cgatgacaagtgaaaaaggaccctcaactggggacccaaccctgcgtcgt |
| | | | cgcattgaaagctgggagttcgacgttttttatgatccgcgtgagctggc |
| | | | gaaagagacgtgcctgttgtacgagatcaaatggggtatgagccgtaaaa |
| | | | tctggagatccagcggtaaaaacaccaccaatcacgtggaagtgaatttt |
| | | | atcaagaagttcaccagcgaacgtcgtttccacagctccatttcttgttc |
| | | | tattacctggtttctgtcttggtcgccgtgttgggagtgctcccaggcaa |
| | | | ttcgtgaattccttagtcaacatccgggcgtgaccctggtcatctacgtc |
| | | | gcccgtttgttttggcatatggaccagcgcaatcgtcagggtttgcgcga |
| | | | tctggttaacagcggcgttacgatccaaattatgcgtgcgagcgagtatt |
| | | | accactgctggcgcaacttcgtgaactatccaccgggcgatgaagcacat |
| | | | tggccgcaatatccgccgctgtggatgatgctgtacgcgctggaactcca |
| | | | ctgcattatcctgtcgctgccgccttgtctgaagatcagccgacgctggc |
| | | | agaaccacctcgcgtttttccgcctgcacttgcaaaactgccattaccag |
| | | | accatcccgccgcatatcctgttagctaccggtctgattcacccgagcgt |
| | | | tacttggcgttctggaagtgagacacccgggacttcagaatccgcgaccc |
| | | | cggagtctggaatgccaaaacccgctgtagagtcagaattttcaaaagtt |
| | | | ttgaaaaagcactttccgggagaacgtttccgctcgagctatatgaaaag |
| | | | aggcggtaagatcctggctgcccaaggcgaagaggcagttgttgcgtatc |
| | | | tacagggcaagtccgaggaggagcctccgaacttccagccaccggcgaag |
| | | | tgccacgtggtgaccaaatcccgtgacttcgcggaatggccgatcatgaa |
| | | | ggcgtcggaagcgattcaacgttatatctatgcactgtcgaccaccgaac |
| | | | gcgcggcctgtaaaccgggcaaaagtagtgagagccatgcagcgtggttc |
| | | | gccgctaccggtgtcagcaaccacggctacagccacgttcagggtctgaa |
| | | | cctgatttttgatcataccttgggccgttacgatggtgttctgaaaaagg |
| | | | tccaactgcgcaacgaaaaggcgagagcgcgtttagagagcattaacgcg |
| | | | tctagagcagatgaaggtttaccggagattaaagccgaggaggaagaagt |
| | | | ggctacgaacgaaaccggccacctgttgcaaccgccgggtatcaatccga |
| | | | gcttttacgtgtatcagaccattagcccgcaggcataccgtccgcgtgac |
| | | | gagatcgtgctgccgccggagtacgcaggttacgtacgtgacccgaatgc |
| | | | tccgattccgctgggtgtggtgcgtaaccgttgcgatatccaaaaagggt |
| | | | gcccgggctacatcccggaatggcagcgtgaagcaggtaccgcgatcagc |
| | | | ccgaaaacaggcaaggccgtaactgtgccgggcctgtctccgaagaaaaa |
| | | | caaacgtatgcgtaggtactggcgctctgagaaggagaaagctcaagatg |
| | | | cgctgttggttaccgtacgcattggtaccgattgggttgtcatcgacgtg |
| | | | cgcggtctgttgcgcaacgcccgttggcgcaccatcgctcccaaggacat |
| | | | cagcctgaatgcgctcctcgatttgtttaccggtgatccggtgatcgatg |
| | | | tgcgtcgcaatattgttacctttacgtacaccctcgacgcctgtggtacc |
| | | | tacgcccgtaaatggaccttgaaaggcaaacagacgaaagcgaccctgga |
| | | | caaactgacggccactcagacggttgcgttggttgcgattgcgcttggtc |
| | | | agaccaacccgatttccgctggcattagccgtgttacacaagaaaatggt |
| | | | gcactgcaatgcgaaccgctggaccggttcaccctgccggatgacctgtt |
| | | | gaaggacatcagcgcatatcgcattgcttgggacagaaacgaagaggaac |
| | | | tacgcgctcgcagcgtcgaggccctgccggaggcgcaacaagcggaggtc |
| | | | cgtgcgctggacggggtgtccaaagaaaccgcgcgtacccaactgtgcgc |
| | | | agatttcggcctggacccgaaacgtctgccgtgggataaaatgagctcca |
| | | | atactacctttatcagcgaagccttgctgtctaacagcgtgagccgtgat |
| | | | caggtttttttcactccggctccgaagaagggtgctaagaaaaaagcgcc |
| | | | tgttgaagttatgcgtaaagaccgcacttgggcacgtgcgtacaagccgc |
| | | | gtctgagcgtcgaagctcagaaactgaagaacgaagccctgtgggcactt |
| | | | aagcgcacctccccggagtatttgaagctgagccgtcgcaaagaggagct |
| | | | gtgtcgtcgtagcatcaactatgttatcgaaaagacccgtcgccgtacgc |
| | | | aatgtcagattgtgatcccggtgatcgaggatctgaatgttcgcttcttt |
| | | | catggttctggtaagagactgcccggttgggataatttcttcaccgctaa |
| | | | aaaagagaaccgttggttcatccagggcctgcacaaagcgtttagcgatc |
| | | | tgcgtacgcaccgttccttctatgtttttgaagttcgtccggagcgcacc |
| | | | tctattacctgtccaaagtgcggtcattgcgaggtgggtaatcgtgacgg |
| | | | tgaggcgtttcagtgcctgagctgcggcaagacgtgcaacgcagatcttg |
| | | | acgtcgcgacccataatctgacccaagtggcgttgaccggtaagaccatg |
| | | | ccgaagcgcgaggagccgcgtgacgctcagggtacagcgccagcgcgcaa |
| | | | gactaagaaggcgagcaaatcaaaggcgccaccggcggagcgcgaagacc |
| | | | agaccccggcgcaagagccaagccagaccagttctggaggtagcgggggt |
| | | | agcggtggtagcactaacttaagtgatattatcgagaaagaaaccgggaa |
| | | | acagctggtgatccaagaaagcattctgatgctgcccgaagaagttgaag |
| | | | aagtgattggtaacaagcctgagtcggacatcttagtgcacaccgcgtac |
| | | | gatgaaagcacggatgagaatgtgatgctgctcacctctgatgcccccga |
| | | | atacaagccgtgggctctggtgatccaggactctaacggagaaaacaaaa |
| | | | ttaagatgctctctggcggctccatggcgcctaaaaaaaaacgcaaagtg |
| | | | gggattcacggggttccggcgtaa |
| 45 | Base editor | PpA1-R33A- | atgggaagtcatcaccaccaccaccattcaggtgagaacctgtacttcca |
| | | dLbCas12a | gagcatggctccgaaaaagaaacgtaaggtgggtatccacggcgttccgg |
| | | | cgatgacaagtgaaaaaggaccctcaactggggacccaaccctgcgtcgt |
| | | | cgcattgaaagctgggagttcgacgttttttatgatccgcgtgagctggc |
| | | | gaaagagacgtgcctgttgtacgagatcaaatggggtatgagccgtaaaa |
| | | | tctggagatccagcggtaaaaacaccaccaatcacgtggaagtgaatttt |
| | | | atcaagaagttcaccagcgaacgtcgtttccacagctccatttcttgttc |
| | | | tattacctggtttctgtcttggtcgccgtgttgggagtgctcccaggcaa |
| | | | ttcgtgaattccttagtcaacatccgggcgtgaccctggtcatctacgtc |
| | | | gcccgtttgttttggcatatggaccagcgcaatcgtcagggtttgcgcga |
| | | | tctggttaacagcggcgttacgatccaaattatgcgtgcgagcgagtatt |
| | | | accactgctggcgcaacttcgtgaactatccaccgggcgatgaagcacat |
| | | | tggccgcaatatccgccgctgtggatgatgctgtacgcgctggaactcca |
| | | | ctgcattatcctgtcgctgccgccttgtctgaagatcagccgacgctggc |
| | | | agaaccacctcgcgtttttccgcctgcacttgcaaaactgccattaccag |
| | | | accatcccgccgcatatcctgttagctaccggtctgattcacccgagcgt |
| | | | tacttggcgttctggaagtgagacacccgggacttcagaatccgcgaccc |
| | | | cggagtctggaatgtcaaaactagaaaagtttacaaattgttactccctg |
| | | | tcgaaaaccctgagattcaaagcgatcccggtgggtaaaactcaagagaa |
| | | | cattgataacaaacgtctgctggttgaggacgaaaagcgcgcggaggatt |
| | | | acaaaggcgttaaaaagctgttggaccgttattacctgtcctttattaat |
| | | | gatgttctgcacagcattaaattaaagaacctaaacaattacatctccct |
| | | | cttccgtaaaaagacccgcaccgaaaaggaaaataaagaactggagaacc |
| | | | ttgagattaatctgcgtaaagaaatcgctaaagcgtttaaaggcaacgag |
| | | | ggttataaatccttatttaaaaaagacatcatcgaaacgattctcccaga |
| | | | attcctggacgacaaagacgagatcgcgctggttaattccttcaacggct |
| | | | ttaccaccgcattcaccggcttttttgataaccgcgaaaacatgttctct |
| | | | gaagaggccaaaagcacctcaattgcgtttcgctgcattaacgaaaatct |
| | | | gacgcgttatatctctaacatggatatcttcgagaaggttgacgctatct |
| | | | ttgataaacatgaagtccaggagatcaaggagaagatcctgaacagcgac |
| | | | tacgacgtggaggatttcttcgagggtgagttcttcaattttgttttgac |
| | | | ccaagaaggcatcgatgtctacaatgcgataattggtggttttgttaccg |
| | | | agagcggggagaaaattaagggtctgaacgaatatatcaacttgtacaac |
| | | | cagaaaaccaaacaaaaactgccgaagtttaagccgctgtacaagcaagt |
| | | | cttgtcggatcgtgaatcgctgagcttttacggcgaaggctacaccagcg |
| | | | atgaggaggtgttggaggtgttccgcaatacgttgaataagaactctgaa |
| | | | attttttccagcattaagaagctggaaaaactattcaagaacttcgacga |
| | | | gtatagcagcgcaggtatctttgtgaaaaacggtccggctattagcacca |
| | | | tctcgaaagacattttcggcgaatggaatgttattcgtgataagtggaat |
| | | | gccgaatacgacgacattcatctgaagaaaaaggcggttgtaactgaaaa |
| | | | gtacgaggacgatcgtcgtaagtcgttcaaaaagatcggctccttctctc |
| | | | tggagcaactgcaagagtacgccgatgccgatttgtcagtcgtggagaag |
| | | | ctgaaagagatcatcattcaaaaagttgacgaaatctataaggtctatgg |
| | | | tagcagtgaaaaactgtttgacgcggacttcgtgctggagaaaagcctga |
| | | | agaaaaatgacgccgtggtggcaatcatgaaagatcttctggatagcgtc |
| | | | aagtctttcgaaaactacatcaaagcgtttttcggtgaaggcaaggaaac |
| | | | gaatcgtgacgagagcttctatggtgatttcgtcctagcctatgatatcc |
| | | | tgcttaaggtggatcacatctatgatgccatccgcaactatgttacccag |
| | | | aaaccgtactcaaaggacaagttcaagttatacttccagaatccgcagtt |
| | | | tatgggtggctgggataaagataaggaaaccgactatcgcgccaccattt |
| | | | tacgttacggtagtaagtactatctggcaatcatggataaaaaatatgcg |
| | | | aaatgcctgcaaaagatcgacaaggacgatgttaacggcaattatgagaa |
| | | | gatcaactacaaactgctgccgggcccaaataagatgctgccgaaggtgt |
| | | | ttttcagcaaaaaatggatggcgtattacaatccgagcgaggacatccaa |
| | | | aagatctacaaaaacggtacgtttaagaagggtgatatgttcaacctcaa |
| | | | cgattgtcataaacttattgacttcttcaaggatagcatatctcgttatc |
| | | | cgaagtggagcaacgcgtatgactttaactttagcgaaaccgagaaatac |
| | | | aaagatatcgcgggtttctatcgtgaagttgaagaacagggttacaaagt |
| | | | gtccttcgagtccgcgagcaagaaggaggtagacaagctggtcgaggagg |
| | | | gtaagttgtacatgtttcagatttacaataaggacttttccgacaagtcc |
| | | | catggtacaccgaatttgcacaccatgtatttcaagcttctgttcgacga |
| | | | gaacaaccacggccagatccgtctgagcggtggtgcagagctgttcatgc |
| | | | gtagagctagcctgaagaaagaagagttggtcgttcatccggctaattcg |
| | | | ccgatcgcgaacaaaaatccagataaccctaaaaagactacgaccctgag |
| | | | ctatgatgtgtataaagacaaacgctttagcgaagaccagtatgagttac |
| | | | acattccgatcgctatcaacaagtgcccgaagaatattttcaagatcaac |
| | | | accgaagtgcgtgttcttttgaagcacgatgacaacccgtatgtgattgg |
| | | | tattgctcgtggtgaacgtaatctgctgtacattgtggttgtggacggca |
| | | | aaggaaacatcgtggaacagtacagcctgaacgaaatcattaacaatttc |
| | | | aacggcattcgcattaagacggattaccattctttgctggacaaaaagga |
| | | | gaaagagcgtttcgaggcccgtcagaactggacctccatcgagaacatta |
| | | | aggagctgaaggcgggttacatcagccaggtggtgcacaaaatctgcgaa |
| | | | ctggtggagaagtacgacgcggtgattgctttggcggatttgaactccgg |
| | | | ttttaagaacagccgcgttaaagtggaaaagcaggtttatcagaaattcg |
| | | | aaaaaatgctgattgataaattgaattacatggttgacaaaaagtctaac |
| | | | ccgtgtgcaaccggcggtgcgctgaagggctaccaaatcaccaataagtt |
| | | | tgaaagctttaagagcatgagcacgcaaaatggcttcattttttacattc |
| | | | cggcgtggctgaccagcaagatcgatccgtcaaccggctttgttaattta |
| | | | ctgaagacgaagtatacctctattgccgactctaaaaaatttattagttc |
| | | | cttcgatagaatcatgtatgttccggaagaggacctgttcgaatttgcac |
| | | | tggactacaagaactttagccgtaccgatgctgattatataaagaaatgg |
| | | | aaactctacagctatggcaaccgcatccgtatctttcgcaacccgaaaaa |
| | | | aaacaacgtgttcgactgggaagaggtatgtctgacttcagcgtacaagg |
| | | | agcttttcaataaatatggtatcaactaccagcagggtgatatccgtgca |
| | | | ctgctttgcgagcaaagcgataaagcgttctattcaagcttcatggcgct |
| | | | gatgagcctgatgctgcaaatgcgcaacagcatcaccggtcgtaccgatg |
| | | | ttgcatttctgatttctccagtaaaaaacagcgatggtatcttttacgac |
| | | | agccgtaattacgaggcccaagaaaatgcgattttgccgaagaacgctga |
| | | | cgcgaatggtgcatacaacattgcaaggaaagttctgtgggcaatcggcc |
| | | | aatttaaaaaagcggaagatgagaagctggacaaagtgaaaattgcgatt |
| | | | agcaacaaggagtggctggagtatgcgcagacctccgttaaatcaggggg |
| | | | aagtggcggaagcggtgggtctacgaacctgagcgacattattgaaaaag |
| | | | aaactggtaaacagttggtgatccaagaatccattctgatgttaccggag |
| | | | gaagtggaagaggtgatcggcaacaagccggagtccgacatcctggttca |
| | | | caccgcgtatgatgagtcgaccgatgagaatgttatgctgctgaccagcg |
| | | | acgccccagagtacaaaccgtgggcgctcgtcatccaggatagcaatggc |
| | | | gaaaacaagatcaagatgttgtctggcggtagcatggctccgaaaaagaa |
| | | | gcgtaaagtaggtattcatggtgttccggcatcaggttaa |
| 46 | Base editor | rA1-dCas9 | atgggaagtcatcaccaccaccaccattcaggtgagaacctgtacttcca |
| | | | gagcatggctccgaaaaagaaacgtaaggtgggtatccacggcgttccgg |
| | | | cgatgagttcagaaacaggacccgttgctgtagacccgaccctgcgtcgc |
| | | | cgcattgagccgcacgagttcgaagtcttttttgatccgcgtgaactgcg |
| | | | caaagaaacctgtctgctgtatgaaattaactggggtggccgtcacagca |
| | | | tttggcgtcacactagtcaaaacaccaataaacacgtggaagttaacttc |
| | | | atcgagaaatttaccaccgagcgctacttctgcccgaatactcgttgttc |
| | | | tattacgtggtttctctcttggtcgccgtgcggcgagtgcagccgtgcga |
| | | | ttaccgagttcctgagccgttacccgcacgttaccctgtttatctatatc |
| | | | gcgcgtctgtaccatcatgcggacccgcgtaaccgtcaaggtctgcgtga |
| | | | tctgatcagcagcggtgttacgatccagattatgacggagcaggagtccg |
| | | | gctattgctggcgcaacttcgtgaactatagcccatccaatgaagctcat |
| | | | tggccgcgctacccgcacctgtgggtacgtttgtacgtgctggaactcta |
| | | | ctgcatcatccttggcctgccaccgtgcttgaatattttgagacgcaagc |
| | | | agccgcaactgaccttcttcaccatcgccctgcaaagctgtcattatcag |
| | | | cgtttgccgcctcacatcttatgggcaaccggtttgaagtctggaagtga |
| | | | gacacccgggacttcagaatccgcgaccccggagtctggaatggataaaa |
| | | | agtattcaataggactagctattggcaccaacagcgttggttgggcagtt |
| | | | atcaccgatgaatataaggtcccgagcaagaagtttaaggtcctgggcaa |
| | | | caccgatcgccacagcataaaaaagaaccttattggtgcgctgctgttcg |
| | | | attctggtgagactgccgaagcgacccgtctgaagagaaccgcgcgtcgt |
| | | | agatacacccggcgcaagaaccgtatctgctatctgcaagaaattttcag |
| | | | caacgagatggctaaggtcgacgactccttctttcatcgcctggaggaaa |
| | | | gctttctggttgaggaagacaaaaagcacgagcgtcatccgattttcggt |
| | | | aacattgttgatgaagttgcctaccacgaaaaataccccacaatttatca |
| | | | tctgcgtaaaaaactggtagactccaccgataaggcggatttacgcctta |
| | | | tctatttggcgctggcgcatatgattaagttccgcggtcattttttgatc |
| | | | gagggcgacctgaatccggataacagcgacgttgacaaactgtttatcca |
| | | | gctggtgcagacctataaccagttgttcgaagagaacccgattaatgcgt |
| | | | ctggcgtggacgcgaaggcgatcctgagcgctcgtctgtccaagtcacgc |
| | | | cgcctggagaacttgatcgcgcaactgccgggtgagaagaaaaacggctt |
| | | | gttcggcaatctgattgctctgtccctaggtttgaccccgaatttcaaat |
| | | | ctaattttgatctggcggaagatgcgaaattacagttgagcaaggacact |
| | | | tacgatgacgatctggacaacctgctcgctcagatcggcgatcagtatgc |
| | | | cgatcttttcctggccgcgaagaacctgagcgatgccatcttgctatcgg |
| | | | acatcttacgtgttaacaccgaaatcaccaaagctccgctaagcgcgtcc |
| | | | atgattaaacgttacgatgaacaccaccaggatctgacgctgctgaaagc |
| | | | gttggtgcgccagcaactgccggaaaaatacaaggaaattttcttcgacc |
| | | | aaagcaagaacggttatgcaggctatatcgacggtggtgcgtcgcaagaa |
| | | | gaattttataaattcatcaaaccgattttggagaagatggatggtaccga |
| | | | ggagctgctggtaaaattgaatcgggaagacctcttgcgaaagcagcgta |
| | | | ctttcgataatggtagcatcccgcaccagattcatctgggcgaacttcac |
| | | | gcaatcctacgtcgtcaagaggatttctacccgtttctgaaggataaccg |
| | | | tgaaaagatcgagaagattctgacctttcgtattccgtattatgtgggtc |
| | | | cgctggcgagaggcaacagccgttttgcgtggatgacccgcaagtcggaa |
| | | | gagactatcacgccgtggaatttcgaggaggtcgtggacaagggtgcatc |
| | | | agcgcaatcttttattgaacgtatgaccaattttgataaaaaccttccaa |
| | | | atgaaaaagtgttgccgaagcacagcttgctgtacgagtatttcaccgtt |
| | | | tacaatgaactgacgaaagtcaagtacgttaccgaaggtatgcgtaagcc |
| | | | agcctttctgtctggggagcaaaaaaaagccattgtggatctgctgttca |
| | | | agaccaatcgcaaggttaccgtcaagcaactgaaagaggattacttcaaa |
| | | | aaaatcgaatgttttgactccgtggaaatcagcggcgttgaagaccgctt |
| | | | taatgcatctctgggcacctaccacgatctactcaagatcatcaaggata |
| | | | aggacttcctggacaacgaagaaaatgaggatatcttggaagatattgtg |
| | | | ctgactttaacgttgttcgaggatcgtgagatgattgaagaacgcctcaa |
| | | | aacgtacgcacatctgtttgacgataaggtgatgaaacagctaaagcgcc |
| | | | gtcgttacaccggttggggtcgtctgtctagaaagctgattaacggcatt |
| | | | cgtgacaaacaatctggcaagaccatcctggacttcctcaaatccgacgg |
| | | | cttcgcaaatcgtaatttcatgcaactgatccatgatgatagcctgacct |
| | | | ttaaagaagatattcagaaggctcaagtgtccggccagggtgactcgctg |
| | | | catgagcatatcgcgaatctggctggctcccctgcgataaaaaaaggtat |
| | | | tctccaaacggtgaaagttgtggatgaactggttaaagtcatgggtcgtc |
| | | | ataaaccggaaaacatcgtgattgagatggcacgtgagaaccagaccacc |
| | | | cagaaaggtcaaaaaaacagtcgtgagcgtatgaaacgtatcgaagaggg |
| | | | gattaaggagttaggcagccagattctgaaggagcacccggttgagaaca |
| | | | cccagctgcaaaacgagaagctctacctgtattacctgcaaaacggccgt |
| | | | gacatgtatgttgaccaggagctggacatcaatcgtctcagcgactacga |
| | | | cgtggatgctatcgtgccgcagagctttctaaaagatgattccatcgaca |
| | | | ataaagtgctcacccgtagcgacaagaaccgtggtaaaagcgacaacgtg |
| | | | ccgagcgaggaagtagttaagaaaatgaaaaactactggcgtcagctatt |
| | | | gaacgccaagcttatcacccagcgtaagtttgacaacttgacgaaggcag |
| | | | agcgtggcggcctgagcgagctagacaaggcgggttttattaaacgccag |
| | | | ctggtggaaactcgccagattaccaaacacgttgcgcaaatcttggactc |
| | | | acgcatgaatacgaagtacgacgaaaacgataaattgatccgcgaggtga |
| | | | aggtcataaccctgaaatctaaactggttagcgatttccggaaggacttt |
| | | | caattttataaagtccgggagattaacaactatcaccatgcacacgacgc |
| | | | ttacttaaatgcggttgttggcacggccctgatcaagaagtacccgaaac |
| | | | tggaaagcgagtttgtttatggtgactacaaggtgtatgacgttcgcaag |
| | | | atgattgcaaaatccgaacaagagattggtaaggcgacggctaagtattt |
| | | | tttctacagcaatatcatgaacttcttcaagactgaaatcaccctggcga |
| | | | acggtgaaattcgtaaacgtccgttgatcgaaaccaacggtgaaaccggt |
| | | | gaaatcgtttgggataaaggtagggattttgcgaccgttcgtaaggtcct |
| | | | gagcatgccgcaggttaacatcgtgaaaaagaccgaagtgcagaccggcg |
| | | | gtttctccaaggagagtatcttaccgaagcgcaacagtgacaagctgatc |
| | | | gcgcgtaaaaaagactgggatccgaaaaaatacggcggctttgactcccc |
| | | | gaccgtcgcgtacagcgtactggtcgtggctaaagtggagaaaggcaaat |
| | | | cgaaaaagctgaaatccgttaaagagttgctgggtattacgattatggaa |
| | | | cgttcgagcttcgagaaaaatccgatcgacttcctggaggccaaaggcta |
| | | | caaggaagttaaaaaagacctgatcattaaactgccgaagtacagcctgt |
| | | | tcgagctcgagaacggtcgcaaacgcatgctggcgagcgcgggtgagctc |
| | | | caaaaaggcaacgaattggctctgccgagcaagtacgtgaacttcctgta |
| | | | tctggcctcccattatgaaaaactgaagggtagcccggaagataacgaac |
| | | | aaaagcagctgttcgtggaacaacataaacactatctggacgagattatt |
| | | | gagcaaatcagcgagttcagcaaacgcgtgattctggcggatgctaatct |
| | | | ggacaaggttctttcagcctataataaacaccgtgataagccgatccgtg |
| | | | aacaggcagaaaatattattcacctgttcaccctgacgaacctgggtgcg |
| | | | ccagcagcgtttaaatacttcgacacaacgatcgaccggaagcgttatac |
| | | | cagcaccaaagaggttcttgacgccaccttgattcaccaaagcatcacgg |
| | | | gtctgtatgaaacccgtattgacttgagccagttgggcggagattcaggg |
| | | | ggaagtggcggaagcggtgggtctacgaacctgagcgacattattgaaaa |
| | | | agaaactggtaaacagttggtgatccaagaatccattctgatgttaccgg |
| | | | aggaagtggaagaggtgatcggcaacaagccggagtccgacatcctggtt |
| | | | cacaccgcgtatgatgagtcgaccgatgagaatgttatgctgctgaccag |
| | | | cgacgccccagagtacaaaccgtgggcgctcgtcatccaggatagcaatg |
| | | | gcgaaaacaagatcaagatgttgtctggcggtagcatggctccgaaaaag |
| | | | aagcgtaaagtaggtattcatggtgttccggcatcaggttaa |
| 47 | Base editor | rA1-dCasPhi-2 | atgggaagtcatcaccaccaccaccattcaggtgagaacctgtacttcca |
| | | | gagcatggctccgaaaaagaaacgtaaggtgggtatccacggcgttccgg |
| | | | cgatgagttcagaaacaggacccgttgctgtagacccgaccctgcgtcgc |
| | | | cgcattgagccgcacgagttcgaagtcttttttgatccgcgtgaactgcg |
| | | | caaagaaacctgtctgctgtatgaaattaactggggtggccgtcacagca |
| | | | tttggcgtcacactagtcaaaacaccaataaacacgtggaagttaacttc |
| | | | atcgagaaatttaccaccgagcgctacttctgcccgaatactcgttgttc |
| | | | tattacgtggtttctctcttggtcgccgtgcggcgagtgcagccgtgcga |
| | | | ttaccgagttcctgagccgttacccgcacgttaccctgtttatctatatc |
| | | | gcgcgtctgtaccatcatgcggacccgcgtaaccgtcaaggtctgcgtga |
| | | | tctgatcagcagcggtgttacgatccagattatgacggagcaggagtccg |
| | | | gctattgctggcgcaacttcgtgaactatagcccatccaatgaagctcat |
| | | | tggccgcgctacccgcacctgtgggtacgtttgtacgtgctggaactcta |
| | | | ctgcatcatccttggcctgccaccgtgcttgaatattttgagacgcaagc |
| | | | agccgcaactgaccttcttcaccatcgccctgcaaagctgtcattatcag |
| | | | cgtttgccgcctcacatcttatgggcaaccggtttgaagtctggaagtga |
| | | | gacacccgggacttcagaatccgcgaccccggagtctggaatgccaaaac |
| | | | ccgctgtagagtcagaattttcaaaagttttgaaaaagcactttccggga |
| | | | gaacgtttccgctcgagctatatgaaaagaggcggtaagatcctggctgc |
| | | | ccaaggcgaagaggcagttgttgcgtatctacagggcaagtccgaggagg |
| | | | agcctccgaacttccagccaccggcgaagtgccacgtggtgaccaaatcc |
| | | | cgtgacttcgcggaatggccgatcatgaaggcgtcggaagcgattcaacg |
| | | | ttatatctatgcactgtcgaccaccgaacgcgcggcctgtaaaccgggca |
| | | | aaagtagtgagagccatgcagcgtggttcgccgctaccggtgtcagcaac |
| | | | cacggctacagccacgttcagggtctgaacctgatttttgatcatacctt |
| | | | gggccgttacgatggtgttctgaaaaaggtccaactgcgcaacgaaaagg |
| | | | cgagagcgcgtttagagagcattaacgcgtctagagcagatgaaggttta |
| | | | ccggagattaaagccgaggaggaagaagtggctacgaacgaaaccggcca |
| | | | cctgttgcaaccgccgggtatcaatccgagcttttacgtgtatcagacca |
| | | | ttagcccgcaggcataccgtccgcgtgacgagatcgtgctgccgccggag |
| | | | tacgcaggttacgtacgtgacccgaatgctccgattccgctgggtgtggt |
| | | | gcgtaaccgttgcgatatccaaaaagggtgcccgggctacatcccggaat |
| | | | ggcagcgtgaagcaggtaccgcgatcagcccgaaaacaggcaaggccgta |
| | | | actgtgccgggcctgtctccgaagaaaaacaaacgtatgcgtaggtactg |
| | | | gcgctctgagaaggagaaagctcaagatgcgctgttggttaccgtacgca |
| | | | ttggtaccgattgggttgtcatcgacgtgcgcggtctgttgcgcaacgcc |
| | | | cgttggcgcaccatcgctcccaaggacatcagcctgaatgcgctcctcga |
| | | | tttgtttaccggtgatccggtgatcgatgtgcgtcgcaatattgttacct |
| | | | ttacgtacaccctcgacgcctgtggtacctacgcccgtaaatggaccttg |
| | | | aaaggcaaacagacgaaagcgaccctggacaaactgacggccactcagac |
| | | | ggttgcgttggttgcgattgcgcttggtcagaccaacccgatttccgctg |
| | | | gcattagccgtgttacacaagaaaatggtgcactgcaatgcgaaccgctg |
| | | | gaccggttcaccctgccggatgacctgttgaaggacatcagcgcatatcg |
| | | | cattgcttgggacagaaacgaagaggaactacgcgctcgcagcgtcgagg |
| | | | ccctgccggaggcgcaacaagcggaggtccgtgcgctggacggggtgtcc |
| | | | aaagaaaccgcgcgtacccaactgtgcgcagatttcggcctggacccgaa |
| | | | acgtctgccgtgggataaaatgagctccaatactacctttatcagcgaag |
| | | | ccttgctgtctaacagcgtgagccgtgatcaggtttttttcactccggct |
| | | | ccgaagaagggtgctaagaaaaaagcgcctgttgaagttatgcgtaaaga |
| | | | ccgcacttgggcacgtgcgtacaagccgcgtctgagcgtcgaagctcaga |
| | | | aactgaagaacgaagccctgtgggcacttaagcgcacctccccggagtat |
| | | | ttgaagctgagccgtcgcaaagaggagctgtgtcgtcgtagcatcaacta |
| | | | tgttatcgaaaagacccgtcgccgtacgcaatgtcagattgtgatcccgg |
| | | | tgatcgaggatctgaatgttcgcttctttcatggttctggtaagagactg |
| | | | cccggttgggataatttcttcaccgctaaaaaagagaaccgttggttcat |
| | | | ccagggcctgcacaaagcgtttagcgatctgcgtacgcaccgttccttct |
| | | | atgtttttgaagttcgtccggagcgcacctctattacctgtccaaagtgc |
| | | | ggtcattgcgaggtgggtaatcgtgacggtgaggcgtttcagtgcctgag |
| | | | ctgcggcaagacgtgcaacgcagatcttgacgtcgcgacccataatctga |
| | | | cccaagtggcgttgaccggtaagaccatgccgaagcgcgaggagccgcgt |
| | | | gacgctcagggtacagcgccagcgcgcaagactaagaaggcgagcaaatc |
| | | | aaaggcgccaccggcggagcgcgaagaccagaccccggcgcaagagccaa |
| | | | gccagaccagttctggaggtagcgggggtagcggtggtagcactaactta |
| | | | agtgatattatcgagaaagaaaccgggaaacagctggtgatccaagaaag |
| | | | cattctgatgctgcccgaagaagttgaagaagtgattggtaacaagcctg |
| | | | agtcggacatcttagtgcacaccgcgtacgatgaaagcacggatgagaat |
| | | | gtgatgctgctcacctctgatgcccccgaatacaagccgtgggctctggt |
| | | | gatccaggactctaacggagaaaacaaaattaagatgctctctggcggct |
| | | | ccatggcgcctaaaaaaaaacgcaaagtggggattcacggggttccggcg |
| | | | taa |
| 48 | Base editor | rA1-dLbCas12a | atgggaagtcatcaccaccaccaccattcaggtgagaacctgtacttcca |
| | | | gagcatggctccgaaaaagaaacgtaaggtgggtatccacggcgttccgg |
| | | | cgatgagttcagaaacaggacccgttgctgtagacccgaccctgcgtcgc |
| | | | cgcattgagccgcacgagttcgaagtcttttttgatccgcgtgaactgcg |
| | | | caaagaaacctgtctgctgtatgaaattaactggggtggccgtcacagca |
| | | | tttggcgtcacactagtcaaaacaccaataaacacgtggaagttaacttc |
| | | | atcgagaaatttaccaccgagcgctacttctgcccgaatactcgttgttc |
| | | | tattacgtggtttctctcttggtcgccgtgcggcgagtgcagccgtgcga |
| | | | ttaccgagttcctgagccgttacccgcacgttaccctgtttatctatatc |
| | | | gcgcgtctgtaccatcatgcggacccgcgtaaccgtcaaggtctgcgtga |
| | | | tctgatcagcagcggtgttacgatccagattatgacggagcaggagtccg |
| | | | gctattgctggcgcaacttcgtgaactatagcccatccaatgaagctcat |
| | | | tggccgcgctacccgcacctgtgggtacgtttgtacgtgctggaactcta |
| | | | ctgcatcatccttggcctgccaccgtgcttgaatattttgagacgcaagc |
| | | | agccgcaactgaccttcttcaccatcgccctgcaaagctgtcattatcag |
| | | | cgtttgccgcctcacatcttatgggcaaccggtttgaagtctggaagtga |
| | | | gacacccgggacttcagaatccgcgaccccggagtctggaatgtcaaaac |
| | | | tagaaaagtttacaaattgttactccctgtcgaaaaccctgagattcaaa |
| | | | gcgatcccggtgggtaaaactcaagagaacattgataacaaacgtctgct |
| | | | ggttgaggacgaaaagcgcgcggaggattacaaaggcgttaaaaagctgt |
| | | | tggaccgttattacctgtcctttattaatgatgttctgcacagcattaaa |
| | | | ttaaagaacctaaacaattacatctccctcttccgtaaaaagacccgcac |
| | | | cgaaaaggaaaataaagaactggagaaccttgagattaatctgcgtaaag |
| | | | aaatcgctaaagcgtttaaaggcaacgagggttataaatccttatttaaa |
| | | | aaagacatcatcgaaacgattctcccagaattcctggacgacaaagacga |
| | | | gatcgcgctggttaattccttcaacggctttaccaccgcattcaccggct |
| | | | tttttgataaccgcgaaaacatgttctctgaagaggccaaaagcacctca |
| | | | attgcgtttcgctgcattaacgaaaatctgacgcgttatatctctaacat |
| | | | ggatatcttcgagaaggttgacgctatctttgataaacatgaagtccagg |
| | | | agatcaaggagaagatcctgaacagcgactacgacgtggaggatttcttc |
| | | | gagggtgagttcttcaattttgttttgacccaagaaggcatcgatgtcta |
| | | | caatgcgataattggtggttttgttaccgagagcggggagaaaattaagg |
| | | | gtctgaacgaatatatcaacttgtacaaccagaaaaccaaacaaaaactg |
| | | | ccgaagtttaagccgctgtacaagcaagtcttgtcggatcgtgaatcgct |
| | | | gagcttttacggcgaaggctacaccagcgatgaggaggtgttggaggtgt |
| | | | tccgcaatacgttgaataagaactctgaaattttttccagcattaagaag |
| | | | ctggaaaaactattcaagaacttcgacgagtatagcagcgcaggtatctt |
| | | | tgtgaaaaacggtccggctattagcaccatctcgaaagacattttcggcg |
| | | | aatggaatgttattcgtgataagtggaatgccgaatacgacgacattcat |
| | | | ctgaagaaaaaggcggttgtaactgaaaagtacgaggacgatcgtcgtaa |
| | | | gtcgttcaaaaagatcggctccttctctctggagcaactgcaagagtacg |
| | | | ccgatgccgatttgtcagtcgtggagaagctgaaagagatcatcattcaa |
| | | | aaagttgacgaaatctataaggtctatggtagcagtgaaaaactgtttga |
| | | | cgcggacttcgtgctggagaaaagcctgaagaaaaatgacgccgtggtgg |
| | | | caatcatgaaagatcttctggatagcgtcaagtctttcgaaaactacatc |
| | | | aaagcgtttttcggtgaaggcaaggaaacgaatcgtgacgagagcttcta |
| | | | tggtgatttcgtcctagcctatgatatcctgcttaaggtggatcacatct |
| | | | atgatgccatccgcaactatgttacccagaaaccgtactcaaaggacaag |
| | | | ttcaagttatacttccagaatccgcagtttatgggtggctgggataaaga |
| | | | taaggaaaccgactatcgcgccaccattttacgttacggtagtaagtact |
| | | | atctggcaatcatggataaaaaatatgcgaaatgcctgcaaaagatcgac |
| | | | aaggacgatgttaacggcaattatgagaagatcaactacaaactgctgcc |
| | | | gggcccaaataagatgctgccgaaggtgtttttcagcaaaaaatggatgg |
| | | | cgtattacaatccgagcgaggacatccaaaagatctacaaaaacggtacg |
| | | | tttaagaagggtgatatgttcaacctcaacgattgtcataaacttattga |
| | | | cttcttcaaggatagcatatctcgttatccgaagtggagcaacgcgtatg |
| | | | actttaactttagcgaaaccgagaaatacaaagatatcgcgggtttctat |
| | | | cgtgaagttgaagaacagggttacaaagtgtccttcgagtccgcgagcaa |
| | | | gaaggaggtagacaagctggtcgaggagggtaagttgtacatgtttcaga |
| | | | tttacaataaggacttttccgacaagtcccatggtacaccgaatttgcac |
| | | | accatgtatttcaagcttctgttcgacgagaacaaccacggccagatccg |
| | | | tctgagcggtggtgcagagctgttcatgcgtagagctagcctgaagaaag |
| | | | aagagttggtcgttcatccggctaattcgccgatcgcgaacaaaaatcca |
| | | | gataaccctaaaaagactacgaccctgagctatgatgtgtataaagacaa |
| | | | acgctttagcgaagaccagtatgagttacacattccgatcgctatcaaca |
| | | | agtgcccgaagaatattttcaagatcaacaccgaagtgcgtgttcttttg |
| | | | aagcacgatgacaacccgtatgtgattggtattgctcgtggtgaacgtaa |
| | | | tctgctgtacattgtggttgtggacggcaaaggaaacatcgtggaacagt |
| | | | acagcctgaacgaaatcattaacaatttcaacggcattcgcattaagacg |
| | | | gattaccattctttgctggacaaaaaggagaaagagcgtttcgaggcccg |
| | | | tcagaactggacctccatcgagaacattaaggagctgaaggcgggttaca |
| | | | tcagccaggtggtgcacaaaatctgcgaactggtggagaagtacgacgcg |
| | | | gtgattgctttggcggatttgaactccggttttaagaacagccgcgttaa |
| | | | agtggaaaagcaggtttatcagaaattcgaaaaaatgctgattgataaat |
| | | | tgaattacatggttgacaaaaagtctaacccgtgtgcaaccggcggtgcg |
| | | | ctgaagggctaccaaatcaccaataagtttgaaagctttaagagcatgag |
| | | | cacgcaaaatggcttcattttttacattccggcgtggctgaccagcaaga |
| | | | tcgatccgtcaaccggctttgttaatttactgaagacgaagtatacctct |
| | | | attgccgactctaaaaaatttattagttccttcgatagaatcatgtatgt |
| | | | tccggaagaggacctgttcgaatttgcactggactacaagaactttagcc |
| | | | gtaccgatgctgattatataaagaaatggaaactctacagctatggcaac |
| | | | cgcatccgtatctttcgcaacccgaaaaaaaacaacgtgttcgactggga |
| | | | agaggtatgtctgacttcagcgtacaaggagcttttcaataaatatggta |
| | | | tcaactaccagcagggtgatatccgtgcactgctttgcgagcaaagcgat |
| | | | aaagcgttctattcaagcttcatggcgctgatgagcctgatgctgcaaat |
| | | | gcgcaacagcatcaccggtcgtaccgatgttgcatttctgatttctccag |
| | | | taaaaaacagcgatggtatcttttacgacagccgtaattacgaggcccaa |
| | | | gaaaatgcgattttgccgaagaacgctgacgcgaatggtgcatacaacat |
| | | | tgcaaggaaagttctgtgggcaatcggccaatttaaaaaagcggaagatg |
| | | | agaagctggacaaagtgaaaattgcgattagcaacaaggagtggctggag |
| | | | tatgcgcagacctccgttaaatcagggggaagtggcggaagcggtgggtc |
| | | | tacgaacctgagcgacattattgaaaaagaaactggtaaacagttggtga |
| | | | tccaagaatccattctgatgttaccggaggaagtggaagaggtgatcggc |
| | | | aacaagccggagtccgacatcctggttcacaccgcgtatgatgagtcgac |
| | | | cgatgagaatgttatgctgctgaccagcgacgccccagagtacaaaccgt |
| | | | gggcgctcgtcatccaggatagcaatggcgaaaacaagatcaagatgttg |
| | | | tctggcggtagcatggctccgaaaaagaagcgtaaagtaggtattcatgg |
| | | | tgttccggcatcaggttaa |
| 49 | Base editor | rA1-YE1-dCas9 | atgggaagtcatcaccaccaccaccattcaggtgagaacctgtacttcca |
| | | | gagcatggctccgaaaaagaaacgtaaggtgggtatccacggcgttccgg |
| | | | cgatgagttcagaaacaggacccgttgctgtagacccgaccttgcgcaga |
| | | | cgtattgagccgcacgaattcgaggtgtttttcgacccgagagaattgcg |
| | | | caaagaaacctgtctgctgtacgagatcaactggggtggtcgtcatagca |
| | | | tctggcgtcacacctctcagaatacgaacaaacacgtggaagtcaatttc |
| | | | atcgagaagttcacgaccgagcgctacttctgtccgaacacccgttgttc |
| | | | gatcacgtggtttctgagctattctccgtgcggcgaatgcagccgtgcaa |
| | | | ttaccgagttcctgtcacgttacccgcacgtaactctgtttatctacatt |
| | | | gcgcgtctctaccatcatgctgatccggaaaaccgtcaaggtttacgcga |
| | | | tctgattagctccggcgtgaccatccagattatgactgagcaggagagcg |
| | | | gctattgctggcgcaactttgttaactattccccgtccaatgaggcgcat |
| | | | tggcctcgttatccgcacctgtgggttcgtctatacgttctggaattgta |
| | | | ctgcattatcctgggcctgccaccatgcttgaatattctgcgtcgcaagc |
| | | | agccgcaattgacctttttcaccatcgccctgcaaagctgccattatcag |
| | | | cgccttccgccgcacatcctgtgggcgaccggtctgaaatctggaagtga |
| | | | gacacccgggacttcagaatccgcgaccccggagtctggaatggataaaa |
| | | | agtattcaataggactagctattggcaccaacagcgttggttgggcagtt |
| | | | atcaccgatgaatataaggtcccgagcaagaagtttaaggtcctgggcaa |
| | | | caccgatcgccacagcataaaaaagaaccttattggtgcgctgctgttcg |
| | | | attctggtgagactgccgaagcgacccgtctgaagagaaccgcgcgtcgt |
| | | | agatacacccggcgcaagaaccgtatctgctatctgcaagaaattttcag |
| | | | caacgagatggctaaggtcgacgactccttctttcatcgcctggaggaaa |
| | | | gctttctggttgaggaagacaaaaagcacgagcgtcatccgattttcggt |
| | | | aacattgttgatgaagttgcctaccacgaaaaataccccacaatttatca |
| | | | tctgcgtaaaaaactggtagactccaccgataaggcggatttacgcctta |
| | | | tctatttggcgctggcgcatatgattaagttccgcggtcattttttgatc |
| | | | gagggcgacctgaatccggataacagcgacgttgacaaactgtttatcca |
| | | | gctggtgcagacctataaccagttgttcgaagagaacccgattaatgcgt |
| | | | ctggcgtggacgcgaaggcgatcctgagcgctcgtctgtccaagtcacgc |
| | | | cgcctggagaacttgatcgcgcaactgccgggtgagaagaaaaacggctt |
| | | | gttcggcaatctgattgctctgtccctaggtttgaccccgaatttcaaat |
| | | | ctaattttgatctggcggaagatgcgaaattacagttgagcaaggacact |
| | | | tacgatgacgatctggacaacctgctcgctcagatcggcgatcagtatgc |
| | | | cgatcttttcctggccgcgaagaacctgagcgatgccatcttgctatcgg |
| | | | acatcttacgtgttaacaccgaaatcaccaaagctccgctaagcgcgtcc |
| | | | atgattaaacgttacgatgaacaccaccaggatctgacgctgctgaaagc |
| | | | gttggtgcgccagcaactgccggaaaaatacaaggaaattttcttcgacc |
| | | | aaagcaagaacggttatgcaggctatatcgacggtggtgcgtcgcaagaa |
| | | | gaattttataaattcatcaaaccgattttggagaagatggatggtaccga |
| | | | ggagctgctggtaaaattgaatcgggaagacctcttgcgaaagcagcgta |
| | | | ctttcgataatggtagcatcccgcaccagattcatctgggcgaacttcac |
| | | | gcaatcctacgtcgtcaagaggatttctacccgtttctgaaggataaccg |
| | | | tgaaaagatcgagaagattctgacctttcgtattccgtattatgtgggtc |
| | | | cgctggcgagaggcaacagccgttttgcgtggatgacccgcaagtcggaa |
| | | | gagactatcacgccgtggaatttcgaggaggtcgtggacaagggtgcatc |
| | | | agcgcaatcttttattgaacgtatgaccaattttgataaaaaccttccaa |
| | | | atgaaaaagtgttgccgaagcacagcttgctgtacgagtatttcaccgtt |
| | | | tacaatgaactgacgaaagtcaagtacgttaccgaaggtatgcgtaagcc |
| | | | agcctttctgtctggggagcaaaaaaaagccattgtggatctgctgttca |
| | | | agaccaatcgcaaggttaccgtcaagcaactgaaagaggattacttcaaa |
| | | | aaaatcgaatgttttgactccgtggaaatcagcggcgttgaagaccgctt |
| | | | taatgcatctctgggcacctaccacgatctactcaagatcatcaaggata |
| | | | aggacttcctggacaacgaagaaaatgaggatatcttggaagatattgtg |
| | | | ctgactttaacgttgttcgaggatcgtgagatgattgaagaacgcctcaa |
| | | | aacgtacgcacatctgtttgacgataaggtgatgaaacagctaaagcgcc |
| | | | gtcgttacaccggttggggtcgtctgtctagaaagctgattaacggcatt |
| | | | cgtgacaaacaatctggcaagaccatcctggacttcctcaaatccgacgg |
| | | | cttcgcaaatcgtaatttcatgcaactgatccatgatgatagcctgacct |
| | | | ttaaagaagatattcagaaggctcaagtgtccggccagggtgactcgctg |
| | | | catgagcatatcgcgaatctggctggctcccctgcgataaaaaaaggtat |
| | | | tctccaaacggtgaaagttgtggatgaactggttaaagtcatgggtcgtc |
| | | | ataaaccggaaaacatcgtgattgagatggcacgtgagaaccagaccacc |
| | | | cagaaaggtcaaaaaaacagtcgtgagcgtatgaaacgtatcgaagaggg |
| | | | gattaaggagttaggcagccagattctgaaggagcacccggttgagaaca |
| | | | cccagctgcaaaacgagaagctctacctgtattacctgcaaaacggccgt |
| | | | gacatgtatgttgaccaggagctggacatcaatcgtctcagcgactacga |
| | | | cgtggatgctatcgtgccgcagagctttctaaaagatgattccatcgaca |
| | | | ataaagtgctcacccgtagcgacaagaaccgtggtaaaagcgacaacgtg |
| | | | ccgagcgaggaagtagttaagaaaatgaaaaactactggcgtcagctatt |
| | | | gaacgccaagcttatcacccagcgtaagtttgacaacttgacgaaggcag |
| | | | agcgtggcggcctgagcgagctagacaaggcgggttttattaaacgccag |
| | | | ctggtggaaactcgccagattaccaaacacgttgcgcaaatcttggactc |
| | | | acgcatgaatacgaagtacgacgaaaacgataaattgatccgcgaggtga |
| | | | aggtcataaccctgaaatctaaactggttagcgatttccggaaggacttt |
| | | | caattttataaagtccgggagattaacaactatcaccatgcacacgacgc |
| | | | ttacttaaatgcggttgttggcacggccctgatcaagaagtacccgaaac |
| | | | tggaaagcgagtttgtttatggtgactacaaggtgtatgacgttcgcaag |
| | | | atgattgcaaaatccgaacaagagattggtaaggcgacggctaagtattt |
| | | | tttctacagcaatatcatgaacttcttcaagactgaaatcaccctggcga |
| | | | acggtgaaattcgtaaacgtccgttgatcgaaaccaacggtgaaaccggt |
| | | | gaaatcgtttgggataaaggtagggattttgcgaccgttcgtaaggtcct |
| | | | gagcatgccgcaggttaacatcgtgaaaaagaccgaagtgcagaccggcg |
| | | | gtttctccaaggagagtatcttaccgaagcgcaacagtgacaagctgatc |
| | | | gcgcgtaaaaaagactgggatccgaaaaaatacggcggctttgactcccc |
| | | | gaccgtcgcgtacagcgtactggtcgtggctaaagtggagaaaggcaaat |
| | | | cgaaaaagctgaaatccgttaaagagttgctgggtattacgattatggaa |
| | | | cgttcgagcttcgagaaaaatccgatcgacttcctggaggccaaaggcta |
| | | | caaggaagttaaaaaagacctgatcattaaactgccgaagtacagcctgt |
| | | | tcgagctcgagaacggtcgcaaacgcatgctggcgagcgcgggtgagctc |
| | | | caaaaaggcaacgaattggctctgccgagcaagtacgtgaacttcctgta |
| | | | tctggcctcccattatgaaaaactgaagggtagcccggaagataacgaac |
| | | | aaaagcagctgttcgtggaacaacataaacactatctggacgagattatt |
| | | | gagcaaatcagcgagttcagcaaacgcgtgattctggcggatgctaatct |
| | | | ggacaaggttctttcagcctataataaacaccgtgataagccgatccgtg |
| | | | aacaggcagaaaatattattcacctgttcaccctgacgaacctgggtgcg |
| | | | ccagcagcgtttaaatacttcgacacaacgatcgaccggaagcgttatac |
| | | | cagcaccaaagaggttcttgacgccaccttgattcaccaaagcatcacgg |
| | | | gtctgtatgaaacccgtattgacttgagccagttgggcggagattcaggg |
| | | | ggaagtggcggaagcggtgggtctacgaacctgagcgacattattgaaaa |
| | | | agaaactggtaaacagttggtgatccaagaatccattctgatgttaccgg |
| | | | aggaagtggaagaggtgatcggcaacaagccggagtccgacatcctggtt |
| | | | cacaccgcgtatgatgagtcgaccgatgagaatgttatgctgctgaccag |
| | | | cgacgccccagagtacaaaccgtgggcgctcgtcatccaggatagcaatg |
| | | | gcgaaaacaagatcaagatgttgtctggcggtagcatggctccgaaaaag |
| | | | aagcgtaaagtaggtattcatggtgttccggcatcaggttaa |
| 50 | Base editor | rA1-YE1- | atgggaagtcatcaccaccaccaccattcaggtgagaacctgtacttcca |
| | | dCasPhi-2 | gagcatggctccgaaaaagaaacgtaaggtgggtatccacggcgttccgg |
| | | | cgatgagttcagaaacaggacccgttgctgtagacccgaccttgcgcaga |
| | | | cgtattgagccgcacgaattcgaggtgtttttcgacccgagagaattgcg |
| | | | caaagaaacctgtctgctgtacgagatcaactggggtggtcgtcatagca |
| | | | tctggcgtcacacctctcagaatacgaacaaacacgtggaagtcaatttc |
| | | | atcgagaagttcacgaccgagcgctacttctgtccgaacacccgttgttc |
| | | | gatcacgtggtttctgagctattctccgtgcggcgaatgcagccgtgcaa |
| | | | ttaccgagttcctgtcacgttacccgcacgtaactctgtttatctacatt |
| | | | gcgcgtctctaccatcatgctgatccggaaaaccgtcaaggtttacgcga |
| | | | tctgattagctccggcgtgaccatccagattatgactgagcaggagagcg |
| | | | gctattgctggcgcaactttgttaactattccccgtccaatgaggcgcat |
| | | | tggcctcgttatccgcacctgtgggttcgtctatacgttctggaattgta |
| | | | ctgcattatcctgggcctgccaccatgcttgaatattctgcgtcgcaagc |
| | | | agccgcaattgacctttttcaccatcgccctgcaaagctgccattatcag |
| | | | cgccttccgccgcacatcctgtgggcgaccggtctgaaatctggaagtga |
| | | | gacacccgggacttcagaatccgcgaccccggagtctggaatgccaaaac |
| | | | ccgctgtagagtcagaattttcaaaagttttgaaaaagcactttccggga |
| | | | gaacgtttccgctcgagctatatgaaaagaggcggtaagatcctggctgc |
| | | | ccaaggcgaagaggcagttgttgcgtatctacagggcaagtccgaggagg |
| | | | agcctccgaacttccagccaccggcgaagtgccacgtggtgaccaaatcc |
| | | | cgtgacttcgcggaatggccgatcatgaaggcgtcggaagcgattcaacg |
| | | | ttatatctatgcactgtcgaccaccgaacgcgcggcctgtaaaccgggca |
| | | | aaagtagtgagagccatgcagcgtggttcgccgctaccggtgtcagcaac |
| | | | cacggctacagccacgttcagggtctgaacctgatttttgatcatacctt |
| | | | gggccgttacgatggtgttctgaaaaaggtccaactgcgcaacgaaaagg |
| | | | cgagagcgcgtttagagagcattaacgcgtctagagcagatgaaggttta |
| | | | ccggagattaaagccgaggaggaagaagtggctacgaacgaaaccggcca |
| | | | cctgttgcaaccgccgggtatcaatccgagcttttacgtgtatcagacca |
| | | | ttagcccgcaggcataccgtccgcgtgacgagatcgtgctgccgccggag |
| | | | tacgcaggttacgtacgtgacccgaatgctccgattccgctgggtgtggt |
| | | | gcgtaaccgttgcgatatccaaaaagggtgcccgggctacatcccggaat |
| | | | ggcagcgtgaagcaggtaccgcgatcagcccgaaaacaggcaaggccgta |
| | | | actgtgccgggcctgtctccgaagaaaaacaaacgtatgcgtaggtactg |
| | | | gcgctctgagaaggagaaagctcaagatgcgctgttggttaccgtacgca |
| | | | ttggtaccgattgggttgtcatcgacgtgcgcggtctgttgcgcaacgcc |
| | | | cgttggcgcaccatcgctcccaaggacatcagcctgaatgcgctcctcga |
| | | | tttgtttaccggtgatccggtgatcgatgtgcgtcgcaatattgttacct |
| | | | ttacgtacaccctcgacgcctgtggtacctacgcccgtaaatggaccttg |
| | | | aaaggcaaacagacgaaagcgaccctggacaaactgacggccactcagac |
| | | | ggttgcgttggttgcgattgcgcttggtcagaccaacccgatttccgctg |
| | | | gcattagccgtgttacacaagaaaatggtgcactgcaatgcgaaccgctg |
| | | | gaccggttcaccctgccggatgacctgttgaaggacatcagcgcatatcg |
| | | | cattgcttgggacagaaacgaagaggaactacgcgctcgcagcgtcgagg |
| | | | ccctgccggaggcgcaacaagcggaggtccgtgcgctggacggggtgtcc |
| | | | aaagaaaccgcgcgtacccaactgtgcgcagatttcggcctggacccgaa |
| | | | acgtctgccgtgggataaaatgagctccaatactacctttatcagcgaag |
| | | | ccttgctgtctaacagcgtgagccgtgatcaggtttttttcactccggct |
| | | | ccgaagaagggtgctaagaaaaaagcgcctgttgaagttatgcgtaaaga |
| | | | ccgcacttgggcacgtgcgtacaagccgcgtctgagcgtcgaagctcaga |
| | | | aactgaagaacgaagccctgtgggcacttaagcgcacctccccggagtat |
| | | | ttgaagctgagccgtcgcaaagaggagctgtgtcgtcgtagcatcaacta |
| | | | tgttatcgaaaagacccgtcgccgtacgcaatgtcagattgtgatcccgg |
| | | | tgatcgaggatctgaatgttcgcttctttcatggttctggtaagagactg |
| | | | cccggttgggataatttcttcaccgctaaaaaagagaaccgttggttcat |
| | | | ccagggcctgcacaaagcgtttagcgatctgcgtacgcaccgttccttct |
| | | | atgtttttgaagttcgtccggagcgcacctctattacctgtccaaagtgc |
| | | | ggtcattgcgaggtgggtaatcgtgacggtgaggcgtttcagtgcctgag |
| | | | ctgcggcaagacgtgcaacgcagatcttgacgtcgcgacccataatctga |
| | | | cccaagtggcgttgaccggtaagaccatgccgaagcgcgaggagccgcgt |
| | | | gacgctcagggtacagcgccagcgcgcaagactaagaaggcgagcaaatc |
| | | | aaaggcgccaccggcggagcgcgaagaccagaccccggcgcaagagccaa |
| | | | gccagaccagttctggaggtagcgggggtagcggtggtagcactaactta |
| | | | agtgatattatcgagaaagaaaccgggaaacagctggtgatccaagaaag |
| | | | cattctgatgctgcccgaagaagttgaagaagtgattggtaacaagcctg |
| | | | agtcggacatcttagtgcacaccgcgtacgatgaaagcacggatgagaat |
| | | | gtgatgctgctcacctctgatgcccccgaatacaagccgtgggctctggt |
| | | | gatccaggactctaacggagaaaacaaaattaagatgctctctggcggct |
| | | | ccatggcgcctaaaaaaaaacgcaaagtggggattcacggggttccggcg |
| | | | taa |
| 51 | Base editor | rA1-YE1- | atgggaagtcatcaccaccaccaccattcaggtgagaacctgtacttcca |
| | | dLbCas12a | gagcatggctccgaaaaagaaacgtaaggtgggtatccacggcgttccgg |
| | | | cgatgagttcagaaacaggacccgttgctgtagacccgaccttgcgcaga |
| | | | cgtattgagccgcacgaattcgaggtgtttttcgacccgagagaattgcg |
| | | | caaagaaacctgtctgctgtacgagatcaactggggtggtcgtcatagca |
| | | | tctggcgtcacacctctcagaatacgaacaaacacgtggaagtcaatttc |
| | | | atcgagaagttcacgaccgagcgctacttctgtccgaacacccgttgttc |
| | | | gatcacgtggtttctgagctattctccgtgcggcgaatgcagccgtgcaa |
| | | | ttaccgagttcctgtcacgttacccgcacgtaactctgtttatctacatt |
| | | | gcgcgtctctaccatcatgctgatccggaaaaccgtcaaggtttacgcga |
| | | | tctgattagctccggcgtgaccatccagattatgactgagcaggagagcg |
| | | | gctattgctggcgcaactttgttaactattccccgtccaatgaggcgcat |
| | | | tggcctcgttatccgcacctgtgggttcgtctatacgttctggaattgta |
| | | | ctgcattatcctgggcctgccaccatgcttgaatattctgcgtcgcaagc |
| | | | agccgcaattgacctttttcaccatcgccctgcaaagctgccattatcag |
| | | | cgccttccgccgcacatcctgtgggcgaccggtctgaaatctggaagtga |
| | | | gacacccgggacttcagaatccgcgaccccggagtctggaatgtcaaaac |
| | | | tagaaaagtttacaaattgttactccctgtcgaaaaccctgagattcaaa |
| | | | gcgatcccggtgggtaaaactcaagagaacattgataacaaacgtctgct |
| | | | ggttgaggacgaaaagcgcgcggaggattacaaaggcgttaaaaagctgt |
| | | | tggaccgttattacctgtcctttattaatgatgttctgcacagcattaaa |
| | | | ttaaagaacctaaacaattacatctccctcttccgtaaaaagacccgcac |
| | | | cgaaaaggaaaataaagaactggagaaccttgagattaatctgcgtaaag |
| | | | aaatcgctaaagcgtttaaaggcaacgagggttataaatccttatttaaa |
| | | | aaagacatcatcgaaacgattctcccagaattcctggacgacaaagacga |
| | | | gatcgcgctggttaattccttcaacggctttaccaccgcattcaccggct |
| | | | tttttgataaccgcgaaaacatgttctctgaagaggccaaaagcacctca |
| | | | attgcgtttcgctgcattaacgaaaatctgacgcgttatatctctaacat |
| | | | ggatatcttcgagaaggttgacgctatctttgataaacatgaagtccagg |
| | | | agatcaaggagaagatcctgaacagcgactacgacgtggaggatttcttc |
| | | | gagggtgagttcttcaattttgttttgacccaagaaggcatcgatgtcta |
| | | | caatgcgataattggtggttttgttaccgagagcggggagaaaattaagg |
| | | | gtctgaacgaatatatcaacttgtacaaccagaaaaccaaacaaaaactg |
| | | | ccgaagtttaagccgctgtacaagcaagtcttgtcggatcgtgaatcgct |
| | | | gagcttttacggcgaaggctacaccagcgatgaggaggtgttggaggtgt |
| | | | tccgcaatacgttgaataagaactctgaaattttttccagcattaagaag |
| | | | ctggaaaaactattcaagaacttcgacgagtatagcagcgcaggtatctt |
| | | | tgtgaaaaacggtccggctattagcaccatctcgaaagacattttcggcg |
| | | | aatggaatgttattcgtgataagtggaatgccgaatacgacgacattcat |
| | | | ctgaagaaaaaggcggttgtaactgaaaagtacgaggacgatcgtcgtaa |
| | | | gtcgttcaaaaagatcggctccttctctctggagcaactgcaagagtacg |
| | | | ccgatgccgatttgtcagtcgtggagaagctgaaagagatcatcattcaa |
| | | | aaagttgacgaaatctataaggtctatggtagcagtgaaaaactgtttga |
| | | | cgcggacttcgtgctggagaaaagcctgaagaaaaatgacgccgtggtgg |
| | | | caatcatgaaagatcttctggatagcgtcaagtctttcgaaaactacatc |
| | | | aaagcgtttttcggtgaaggcaaggaaacgaatcgtgacgagagcttcta |
| | | | tggtgatttcgtcctagcctatgatatcctgcttaaggtggatcacatct |
| | | | atgatgccatccgcaactatgttacccagaaaccgtactcaaaggacaag |
| | | | ttcaagttatacttccagaatccgcagtttatgggtggctgggataaaga |
| | | | taaggaaaccgactatcgcgccaccattttacgttacggtagtaagtact |
| | | | atctggcaatcatggataaaaaatatgcgaaatgcctgcaaaagatcgac |
| | | | aaggacgatgttaacggcaattatgagaagatcaactacaaactgctgcc |
| | | | gggcccaaataagatgctgccgaaggtgtttttcagcaaaaaatggatgg |
| | | | cgtattacaatccgagcgaggacatccaaaagatctacaaaaacggtacg |
| | | | tttaagaagggtgatatgttcaacctcaacgattgtcataaacttattga |
| | | | cttcttcaaggatagcatatctcgttatccgaagtggagcaacgcgtatg |
| | | | actttaactttagcgaaaccgagaaatacaaagatatcgcgggtttctat |
| | | | cgtgaagttgaagaacagggttacaaagtgtccttcgagtccgcgagcaa |
| | | | gaaggaggtagacaagctggtcgaggagggtaagttgtacatgtttcaga |
| | | | tttacaataaggacttttccgacaagtcccatggtacaccgaatttgcac |
| | | | accatgtatttcaagcttctgttcgacgagaacaaccacggccagatccg |
| | | | tctgagcggtggtgcagagctgttcatgcgtagagctagcctgaagaaag |
| | | | aagagttggtcgttcatccggctaattcgccgatcgcgaacaaaaatcca |
| | | | gataaccctaaaaagactacgaccctgagctatgatgtgtataaagacaa |
| | | | acgctttagcgaagaccagtatgagttacacattccgatcgctatcaaca |
| | | | agtgcccgaagaatattttcaagatcaacaccgaagtgcgtgttcttttg |
| | | | aagcacgatgacaacccgtatgtgattggtattgctcgtggtgaacgtaa |
| | | | tctgctgtacattgtggttgtggacggcaaaggaaacatcgtggaacagt |
| | | | acagcctgaacgaaatcattaacaatttcaacggcattcgcattaagacg |
| | | | gattaccattctttgctggacaaaaaggagaaagagcgtttcgaggcccg |
| | | | tcagaactggacctccatcgagaacattaaggagctgaaggcgggttaca |
| | | | tcagccaggtggtgcacaaaatctgcgaactggtggagaagtacgacgcg |
| | | | gtgattgctttggcggatttgaactccggttttaagaacagccgcgttaa |
| | | | agtggaaaagcaggtttatcagaaattcgaaaaaatgctgattgataaat |
| | | | tgaattacatggttgacaaaaagtctaacccgtgtgcaaccggcggtgcg |
| | | | ctgaagggctaccaaatcaccaataagtttgaaagctttaagagcatgag |
| | | | cacgcaaaatggcttcattttttacattccggcgtggctgaccagcaaga |
| | | | tcgatccgtcaaccggctttgttaatttactgaagacgaagtatacctct |
| | | | attgccgactctaaaaaatttattagttccttcgatagaatcatgtatgt |
| | | | tccggaagaggacctgttcgaatttgcactggactacaagaactttagcc |
| | | | gtaccgatgctgattatataaagaaatggaaactctacagctatggcaac |
| | | | cgcatccgtatctttcgcaacccgaaaaaaaacaacgtgttcgactggga |
| | | | agaggtatgtctgacttcagcgtacaaggagcttttcaataaatatggta |
| | | | tcaactaccagcagggtgatatccgtgcactgctttgcgagcaaagcgat |
| | | | aaagcgttctattcaagcttcatggcgctgatgagcctgatgctgcaaat |
| | | | gcgcaacagcatcaccggtcgtaccgatgttgcatttctgatttctccag |
| | | | taaaaaacagcgatggtatcttttacgacagccgtaattacgaggcccaa |
| | | | gaaaatgcgattttgccgaagaacgctgacgcgaatggtgcatacaacat |
| | | | tgcaaggaaagttctgtgggcaatcggccaatttaaaaaagcggaagatg |
| | | | agaagctggacaaagtgaaaattgcgattagcaacaaggagtggctggag |
| | | | tatgcgcagacctccgttaaatcagggggaagtggcggaagcggtgggtc |
| | | | tacgaacctgagcgacattattgaaaaagaaactggtaaacagttggtga |
| | | | tccaagaatccattctgatgttaccggaggaagtggaagaggtgatcggc |
| | | | aacaagccggagtccgacatcctggttcacaccgcgtatgatgagtcgac |
| | | | cgatgagaatgttatgctgctgaccagcgacgccccagagtacaaaccgt |
| | | | gggcgctcgtcatccaggatagcaatggcgaaaacaagatcaagatgttg |
| | | | tctggcggtagcatggctccgaaaaagaagcgtaaagtaggtattcatgg |
| | | | tgttccggcatcaggttaa |
| 52 | Base editor | rA1-YEE-dCas9 | atgggaagtcatcaccaccaccaccattcaggtgagaacctgtacttcca |
| | | | gagcatggctccgaaaaagaaacgtaaggtgggtatccacggcgttccgg |
| | | | cgatgtcaagtgaaacaggacccgttgctgtagacccgaccttgagacgc |
| | | | cgcattgagccgcacgaatttgaagttttcttcgacccgcgtgaattgcg |
| | | | taaagagacgtgcttgctgtacgagattaactggggtggccgtcacagca |
| | | | tttggcgccacaccagccaaaacaccaacaaacatgtggaagtcaacttc |
| | | | atcgagaagttcactaccgagcgttacttttgtccgaatacgcgttgttc |
| | | | gatcacctggtttctgtcctattccccgtgcggtgaatgctcacgcgcaa |
| | | | tcactgagttcctgtctcgttacccgcacgtaaccctgtttatctatatc |
| | | | gcgcgtttgtaccatcatgcggatccggagaaccgccagggtctggaaga |
| | | | tttaattagcagcggcgttacgatccagattatgaccgagcaggagagcg |
| | | | gctattgctggcgtaatttcgtgaactattccccgtctaatgaagcgcat |
| | | | tggccgcgttacccacacctgtgggttcgtctctacgtgctggaattgta |
| | | | ctgcattatcctgggtttaccgccttgcctgaatattctacgtcgcaagc |
| | | | aaccgcagctgacctttttcaccatcgccctgcaaagctgtcattatcag |
| | | | cgtctgccgccacacatcctgtgggctaccggccttaaatctggaagtga |
| | | | gacacccgggacttcagaatccgcgaccccggagtctggaatggataaaa |
| | | | agtattcaataggactagctattggcaccaacagcgttggttgggcagtt |
| | | | atcaccgatgaatataaggtcccgagcaagaagtttaaggtcctgggcaa |
| | | | caccgatcgccacagcataaaaaagaaccttattggtgcgctgctgttcg |
| | | | attctggtgagactgccgaagcgacccgtctgaagagaaccgcgcgtcgt |
| | | | agatacacccggcgcaagaaccgtatctgctatctgcaagaaattttcag |
| | | | caacgagatggctaaggtcgacgactccttctttcatcgcctggaggaaa |
| | | | gctttctggttgaggaagacaaaaagcacgagcgtcatccgattttcggt |
| | | | aacattgttgatgaagttgcctaccacgaaaaataccccacaatttatca |
| | | | tctgcgtaaaaaactggtagactccaccgataaggcggatttacgcctta |
| | | | tctatttggcgctggcgcatatgattaagttccgcggtcattttttgatc |
| | | | gagggcgacctgaatccggataacagcgacgttgacaaactgtttatcca |
| | | | gctggtgcagacctataaccagttgttcgaagagaacccgattaatgcgt |
| | | | ctggcgtggacgcgaaggcgatcctgagcgctcgtctgtccaagtcacgc |
| | | | cgcctggagaacttgatcgcgcaactgccgggtgagaagaaaaacggctt |
| | | | gttcggcaatctgattgctctgtccctaggtttgaccccgaatttcaaat |
| | | | ctaattttgatctggcggaagatgcgaaattacagttgagcaaggacact |
| | | | tacgatgacgatctggacaacctgctcgctcagatcggcgatcagtatgc |
| | | | cgatcttttcctggccgcgaagaacctgagcgatgccatcttgctatcgg |
| | | | acatcttacgtgttaacaccgaaatcaccaaagctccgctaagcgcgtcc |
| | | | atgattaaacgttacgatgaacaccaccaggatctgacgctgctgaaagc |
| | | | gttggtgcgccagcaactgccggaaaaatacaaggaaattttcttcgacc |
| | | | aaagcaagaacggttatgcaggctatatcgacggtggtgcgtcgcaagaa |
| | | | gaattttataaattcatcaaaccgattttggagaagatggatggtaccga |
| | | | ggagctgctggtaaaattgaatcgggaagacctcttgcgaaagcagcgta |
| | | | ctttcgataatggtagcatcccgcaccagattcatctgggcgaacttcac |
| | | | gcaatcctacgtcgtcaagaggatttctacccgtttctgaaggataaccg |
| | | | tgaaaagatcgagaagattctgacctttcgtattccgtattatgtgggtc |
| | | | cgctggcgagaggcaacagccgttttgcgtggatgacccgcaagtcggaa |
| | | | gagactatcacgccgtggaatttcgaggaggtcgtggacaagggtgcatc |
| | | | agcgcaatcttttattgaacgtatgaccaattttgataaaaaccttccaa |
| | | | atgaaaaagtgttgccgaagcacagcttgctgtacgagtatttcaccgtt |
| | | | tacaatgaactgacgaaagtcaagtacgttaccgaaggtatgcgtaagcc |
| | | | agcctttctgtctggggagcaaaaaaaagccattgtggatctgctgttca |
| | | | agaccaatcgcaaggttaccgtcaagcaactgaaagaggattacttcaaa |
| | | | aaaatcgaatgttttgactccgtggaaatcagcggcgttgaagaccgctt |
| | | | taatgcatctctgggcacctaccacgatctactcaagatcatcaaggata |
| | | | aggacttcctggacaacgaagaaaatgaggatatcttggaagatattgtg |
| | | | ctgactttaacgttgttcgaggatcgtgagatgattgaagaacgcctcaa |
| | | | aacgtacgcacatctgtttgacgataaggtgatgaaacagctaaagcgcc |
| | | | gtcgttacaccggttggggtcgtctgtctagaaagctgattaacggcatt |
| | | | cgtgacaaacaatctggcaagaccatcctggacttcctcaaatccgacgg |
| | | | cttcgcaaatcgtaatttcatgcaactgatccatgatgatagcctgacct |
| | | | ttaaagaagatattcagaaggctcaagtgtccggccagggtgactcgctg |
| | | | catgagcatatcgcgaatctggctggctcccctgcgataaaaaaaggtat |
| | | | tctccaaacggtgaaagttgtggatgaactggttaaagtcatgggtcgtc |
| | | | ataaaccggaaaacatcgtgattgagatggcacgtgagaaccagaccacc |
| | | | cagaaaggtcaaaaaaacagtcgtgagcgtatgaaacgtatcgaagaggg |
| | | | gattaaggagttaggcagccagattctgaaggagcacccggttgagaaca |
| | | | cccagctgcaaaacgagaagctctacctgtattacctgcaaaacggccgt |
| | | | gacatgtatgttgaccaggagctggacatcaatcgtctcagcgactacga |
| | | | cgtggatgctatcgtgccgcagagctttctaaaagatgattccatcgaca |
| | | | ataaagtgctcacccgtagcgacaagaaccgtggtaaaagcgacaacgtg |
| | | | ccgagcgaggaagtagttaagaaaatgaaaaactactggcgtcagctatt |
| | | | gaacgccaagcttatcacccagcgtaagtttgacaacttgacgaaggcag |
| | | | agcgtggcggcctgagcgagctagacaaggcgggttttattaaacgccag |
| | | | ctggtggaaactcgccagattaccaaacacgttgcgcaaatcttggactc |
| | | | acgcatgaatacgaagtacgacgaaaacgataaattgatccgcgaggtga |
| | | | aggtcataaccctgaaatctaaactggttagcgatttccggaaggacttt |
| | | | caattttataaagtccgggagattaacaactatcaccatgcacacgacgc |
| | | | ttacttaaatgcggttgttggcacggccctgatcaagaagtacccgaaac |
| | | | tggaaagcgagtttgtttatggtgactacaaggtgtatgacgttcgcaag |
| | | | atgattgcaaaatccgaacaagagattggtaaggcgacggctaagtattt |
| | | | tttctacagcaatatcatgaacttcttcaagactgaaatcaccctggcga |
| | | | acggtgaaattcgtaaacgtccgttgatcgaaaccaacggtgaaaccggt |
| | | | gaaatcgtttgggataaaggtagggattttgcgaccgttcgtaaggtcct |
| | | | gagcatgccgcaggttaacatcgtgaaaaagaccgaagtgcagaccggcg |
| | | | gtttctccaaggagagtatcttaccgaagcgcaacagtgacaagctgatc |
| | | | gcgcgtaaaaaagactgggatccgaaaaaatacggcggctttgactcccc |
| | | | gaccgtcgcgtacagcgtactggtcgtggctaaagtggagaaaggcaaat |
| | | | cgaaaaagctgaaatccgttaaagagttgctgggtattacgattatggaa |
| | | | cgttcgagcttcgagaaaaatccgatcgacttcctggaggccaaaggcta |
| | | | caaggaagttaaaaaagacctgatcattaaactgccgaagtacagcctgt |
| | | | tcgagctcgagaacggtcgcaaacgcatgctggcgagcgcgggtgagctc |
| | | | caaaaaggcaacgaattggctctgccgagcaagtacgtgaacttcctgta |
| | | | tctggcctcccattatgaaaaactgaagggtagcccggaagataacgaac |
| | | | aaaagcagctgttcgtggaacaacataaacactatctggacgagattatt |
| | | | gagcaaatcagcgagttcagcaaacgcgtgattctggcggatgctaatct |
| | | | ggacaaggttctttcagcctataataaacaccgtgataagccgatccgtg |
| | | | aacaggcagaaaatattattcacctgttcaccctgacgaacctgggtgcg |
| | | | ccagcagcgtttaaatacttcgacacaacgatcgaccggaagcgttatac |
| | | | cagcaccaaagaggttcttgacgccaccttgattcaccaaagcatcacgg |
| | | | gtctgtatgaaacccgtattgacttgagccagttgggcggagattcaggg |
| | | | ggaagtggcggaagcggtgggtctacgaacctgagcgacattattgaaaa |
| | | | agaaactggtaaacagttggtgatccaagaatccattctgatgttaccgg |
| | | | aggaagtggaagaggtgatcggcaacaagccggagtccgacatcctggtt |
| | | | cacaccgcgtatgatgagtcgaccgatgagaatgttatgctgctgaccag |
| | | | cgacgccccagagtacaaaccgtgggcgctcgtcatccaggatagcaatg |
| | | | gcgaaaacaagatcaagatgttgtctggcggtagcatggctccgaaaaag |
| | | | aagcgtaaagtaggtattcatggtgttccggcatcaggttaa |
| 53 | Base editor | rA1-YEE- | atgggaagtcatcaccaccaccaccattcaggtgagaacctgtacttcca |
| | | dCasPhi-2 | gagcatggctccgaaaaagaaacgtaaggtgggtatccacggcgttccgg |
| | | | cgatgtcaagtgaaacaggacccgttgctgtagacccgaccttgagacgc |
| | | | cgcattgagccgcacgaatttgaagttttcttcgacccgcgtgaattgcg |
| | | | taaagagacgtgcttgctgtacgagattaactggggtggccgtcacagca |
| | | | tttggcgccacaccagccaaaacaccaacaaacatgtggaagtcaacttc |
| | | | atcgagaagttcactaccgagcgttacttttgtccgaatacgcgttgttc |
| | | | gatcacctggtttctgtcctattccccgtgcggtgaatgctcacgcgcaa |
| | | | tcactgagttcctgtctcgttacccgcacgtaaccctgtttatctatatc |
| | | | gcgcgtttgtaccatcatgcggatccggagaaccgccagggtctggaaga |
| | | | tttaattagcagcggcgttacgatccagattatgaccgagcaggagagcg |
| | | | gctattgctggcgtaatttcgtgaactattccccgtctaatgaagcgcat |
| | | | tggccgcgttacccacacctgtgggttcgtctctacgtgctggaattgta |
| | | | ctgcattatcctgggtttaccgccttgcctgaatattctacgtcgcaagc |
| | | | aaccgcagctgacctttttcaccatcgccctgcaaagctgtcattatcag |
| | | | cgtctgccgccacacatcctgtgggctaccggccttaaatctggaagtga |
| | | | gacacccgggacttcagaatccgcgaccccggagtctggaatgccaaaac |
| | | | ccgctgtagagtcagaattttcaaaagttttgaaaaagcactttccggga |
| | | | gaacgtttccgctcgagctatatgaaaagaggcggtaagatcctggctgc |
| | | | ccaaggcgaagaggcagttgttgcgtatctacagggcaagtccgaggagg |
| | | | agcctccgaacttccagccaccggcgaagtgccacgtggtgaccaaatcc |
| | | | cgtgacttcgcggaatggccgatcatgaaggcgtcggaagcgattcaacg |
| | | | ttatatctatgcactgtcgaccaccgaacgcgcggcctgtaaaccgggca |
| | | | aaagtagtgagagccatgcagcgtggttcgccgctaccggtgtcagcaac |
| | | | cacggctacagccacgttcagggtctgaacctgatttttgatcatacctt |
| | | | gggccgttacgatggtgttctgaaaaaggtccaactgcgcaacgaaaagg |
| | | | cgagagcgcgtttagagagcattaacgcgtctagagcagatgaaggttta |
| | | | ccggagattaaagccgaggaggaagaagtggctacgaacgaaaccggcca |
| | | | cctgttgcaaccgccgggtatcaatccgagcttttacgtgtatcagacca |
| | | | ttagcccgcaggcataccgtccgcgtgacgagatcgtgctgccgccggag |
| | | | tacgcaggttacgtacgtgacccgaatgctccgattccgctgggtgtggt |
| | | | gcgtaaccgttgcgatatccaaaaagggtgcccgggctacatcccggaat |
| | | | ggcagcgtgaagcaggtaccgcgatcagcccgaaaacaggcaaggccgta |
| | | | actgtgccgggcctgtctccgaagaaaaacaaacgtatgcgtaggtactg |
| | | | gcgctctgagaaggagaaagctcaagatgcgctgttggttaccgtacgca |
| | | | ttggtaccgattgggttgtcatcgacgtgcgcggtctgttgcgcaacgcc |
| | | | cgttggcgcaccatcgctcccaaggacatcagcctgaatgcgctcctcga |
| | | | tttgtttaccggtgatccggtgatcgatgtgcgtcgcaatattgttacct |
| | | | ttacgtacaccctcgacgcctgtggtacctacgcccgtaaatggaccttg |
| | | | aaaggcaaacagacgaaagcgaccctggacaaactgacggccactcagac |
| | | | ggttgcgttggttgcgattgcgcttggtcagaccaacccgatttccgctg |
| | | | gcattagccgtgttacacaagaaaatggtgcactgcaatgcgaaccgctg |
| | | | gaccggttcaccctgccggatgacctgttgaaggacatcagcgcatatcg |
| | | | cattgcttgggacagaaacgaagaggaactacgcgctcgcagcgtcgagg |
| | | | ccctgccggaggcgcaacaagcggaggtccgtgcgctggacggggtgtcc |
| | | | aaagaaaccgcgcgtacccaactgtgcgcagatttcggcctggacccgaa |
| | | | acgtctgccgtgggataaaatgagctccaatactacctttatcagcgaag |
| | | | ccttgctgtctaacagcgtgagccgtgatcaggtttttttcactccggct |
| | | | ccgaagaagggtgctaagaaaaaagcgcctgttgaagttatgcgtaaaga |
| | | | ccgcacttgggcacgtgcgtacaagccgcgtctgagcgtcgaagctcaga |
| | | | aactgaagaacgaagccctgtgggcacttaagcgcacctccccggagtat |
| | | | ttgaagctgagccgtcgcaaagaggagctgtgtcgtcgtagcatcaacta |
| | | | tgttatcgaaaagacccgtcgccgtacgcaatgtcagattgtgatcccgg |
| | | | tgatcgaggatctgaatgttcgcttctttcatggttctggtaagagactg |
| | | | cccggttgggataatttcttcaccgctaaaaaagagaaccgttggttcat |
| | | | ccagggcctgcacaaagcgtttagcgatctgcgtacgcaccgttccttct |
| | | | atgtttttgaagttcgtccggagcgcacctctattacctgtccaaagtgc |
| | | | ggtcattgcgaggtgggtaatcgtgacggtgaggcgtttcagtgcctgag |
| | | | ctgcggcaagacgtgcaacgcagatcttgacgtcgcgacccataatctga |
| | | | cccaagtggcgttgaccggtaagaccatgccgaagcgcgaggagccgcgt |
| | | | gacgctcagggtacagcgccagcgcgcaagactaagaaggcgagcaaatc |
| | | | aaaggcgccaccggcggagcgcgaagaccagaccccggcgcaagagccaa |
| | | | gccagaccagttctggaggtagcgggggtagcggtggtagcactaactta |
| | | | agtgatattatcgagaaagaaaccgggaaacagctggtgatccaagaaag |
| | | | cattctgatgctgcccgaagaagttgaagaagtgattggtaacaagcctg |
| | | | agtcggacatcttagtgcacaccgcgtacgatgaaagcacggatgagaat |
| | | | gtgatgctgctcacctctgatgcccccgaatacaagccgtgggctctggt |
| | | | gatccaggactctaacggagaaaacaaaattaagatgctctctggcggct |
| | | | ccatggcgcctaaaaaaaaacgcaaagtggggattcacggggttccggcg |
| | | | taa |
| 54 | Base editor | rA1-YEE- | atgggaagtcatcaccaccaccaccattcaggtgagaacctgtacttcca |
| | | dLbCas12a | gagcatggctccgaaaaagaaacgtaaggtgggtatccacggcgttccgg |
| | | | cgatgtcaagtgaaacaggacccgttgctgtagacccgaccttgagacgc |
| | | | cgcattgagccgcacgaatttgaagttttcttcgacccgcgtgaattgcg |
| | | | taaagagacgtgcttgctgtacgagattaactggggtggccgtcacagca |
| | | | tttggcgccacaccagccaaaacaccaacaaacatgtggaagtcaacttc |
| | | | atcgagaagttcactaccgagcgttacttttgtccgaatacgcgttgttc |
| | | | gatcacctggtttctgtcctattccccgtgcggtgaatgctcacgcgcaa |
| | | | tcactgagttcctgtctcgttacccgcacgtaaccctgtttatctatatc |
| | | | gcgcgtttgtaccatcatgcggatccggagaaccgccagggtctggaaga |
| | | | tttaattagcagcggcgttacgatccagattatgaccgagcaggagagcg |
| | | | gctattgctggcgtaatttcgtgaactattccccgtctaatgaagcgcat |
| | | | tggccgcgttacccacacctgtgggttcgtctctacgtgctggaattgta |
| | | | ctgcattatcctgggtttaccgccttgcctgaatattctacgtcgcaagc |
| | | | aaccgcagctgacctttttcaccatcgccctgcaaagctgtcattatcag |
| | | | cgtctgccgccacacatcctgtgggctaccggccttaaatctggaagtga |
| | | | gacacccgggacttcagaatccgcgaccccggagtctggaatgtcaaaac |
| | | | tagaaaagtttacaaattgttactccctgtcgaaaaccctgagattcaaa |
| | | | gcgatcccggtgggtaaaactcaagagaacattgataacaaacgtctgct |
| | | | ggttgaggacgaaaagcgcgcggaggattacaaaggcgttaaaaagctgt |
| | | | tggaccgttattacctgtcctttattaatgatgttctgcacagcattaaa |
| | | | ttaaagaacctaaacaattacatctccctcttccgtaaaaagacccgcac |
| | | | cgaaaaggaaaataaagaactggagaaccttgagattaatctgcgtaaag |
| | | | aaatcgctaaagcgtttaaaggcaacgagggttataaatccttatttaaa |
| | | | aaagacatcatcgaaacgattctcccagaattcctggacgacaaagacga |
| | | | gatcgcgctggttaattccttcaacggctttaccaccgcattcaccggct |
| | | | tttttgataaccgcgaaaacatgttctctgaagaggccaaaagcacctca |
| | | | attgcgtttcgctgcattaacgaaaatctgacgcgttatatctctaacat |
| | | | ggatatcttcgagaaggttgacgctatctttgataaacatgaagtccagg |
| | | | agatcaaggagaagatcctgaacagcgactacgacgtggaggatttcttc |
| | | | gagggtgagttcttcaattttgttttgacccaagaaggcatcgatgtcta |
| | | | caatgcgataattggtggttttgttaccgagagcggggagaaaattaagg |
| | | | gtctgaacgaatatatcaacttgtacaaccagaaaaccaaacaaaaactg |
| | | | ccgaagtttaagccgctgtacaagcaagtcttgtcggatcgtgaatcgct |
| | | | gagcttttacggcgaaggctacaccagcgatgaggaggtgttggaggtgt |
| | | | tccgcaatacgttgaataagaactctgaaattttttccagcattaagaag |
| | | | ctggaaaaactattcaagaacttcgacgagtatagcagcgcaggtatctt |
| | | | tgtgaaaaacggtccggctattagcaccatctcgaaagacattttcggcg |
| | | | aatggaatgttattcgtgataagtggaatgccgaatacgacgacattcat |
| | | | ctgaagaaaaaggcggttgtaactgaaaagtacgaggacgatcgtcgtaa |
| | | | gtcgttcaaaaagatcggctccttctctctggagcaactgcaagagtacg |
| | | | ccgatgccgatttgtcagtcgtggagaagctgaaagagatcatcattcaa |
| | | | aaagttgacgaaatctataaggtctatggtagcagtgaaaaactgtttga |
| | | | cgcggacttcgtgctggagaaaagcctgaagaaaaatgacgccgtggtgg |
| | | | caatcatgaaagatcttctggatagcgtcaagtctttcgaaaactacatc |
| | | | aaagcgtttttcggtgaaggcaaggaaacgaatcgtgacgagagcttcta |
| | | | tggtgatttcgtcctagcctatgatatcctgcttaaggtggatcacatct |
| | | | atgatgccatccgcaactatgttacccagaaaccgtactcaaaggacaag |
| | | | ttcaagttatacttccagaatccgcagtttatgggtggctgggataaaga |
| | | | taaggaaaccgactatcgcgccaccattttacgttacggtagtaagtact |
| | | | atctggcaatcatggataaaaaatatgcgaaatgcctgcaaaagatcgac |
| | | | aaggacgatgttaacggcaattatgagaagatcaactacaaactgctgcc |
| | | | gggcccaaataagatgctgccgaaggtgtttttcagcaaaaaatggatgg |
| | | | cgtattacaatccgagcgaggacatccaaaagatctacaaaaacggtacg |
| | | | tttaagaagggtgatatgttcaacctcaacgattgtcataaacttattga |
| | | | cttcttcaaggatagcatatctcgttatccgaagtggagcaacgcgtatg |
| | | | actttaactttagcgaaaccgagaaatacaaagatatcgcgggtttctat |
| | | | cgtgaagttgaagaacagggttacaaagtgtccttcgagtccgcgagcaa |
| | | | gaaggaggtagacaagctggtcgaggagggtaagttgtacatgtttcaga |
| | | | tttacaataaggacttttccgacaagtcccatggtacaccgaatttgcac |
| | | | accatgtatttcaagcttctgttcgacgagaacaaccacggccagatccg |
| | | | tctgagcggtggtgcagagctgttcatgcgtagagctagcctgaagaaag |
| | | | aagagttggtcgttcatccggctaattcgccgatcgcgaacaaaaatcca |
| | | | gataaccctaaaaagactacgaccctgagctatgatgtgtataaagacaa |
| | | | acgctttagcgaagaccagtatgagttacacattccgatcgctatcaaca |
| | | | agtgcccgaagaatattttcaagatcaacaccgaagtgcgtgttcttttg |
| | | | aagcacgatgacaacccgtatgtgattggtattgctcgtggtgaacgtaa |
| | | | tctgctgtacattgtggttgtggacggcaaaggaaacatcgtggaacagt |
| | | | acagcctgaacgaaatcattaacaatttcaacggcattcgcattaagacg |
| | | | gattaccattctttgctggacaaaaaggagaaagagcgtttcgaggcccg |
| | | | tcagaactggacctccatcgagaacattaaggagctgaaggcgggttaca |
| | | | tcagccaggtggtgcacaaaatctgcgaactggtggagaagtacgacgcg |
| | | | gtgattgctttggcggatttgaactccggttttaagaacagccgcgttaa |
| | | | agtggaaaagcaggtttatcagaaattcgaaaaaatgctgattgataaat |
| | | | tgaattacatggttgacaaaaagtctaacccgtgtgcaaccggcggtgcg |
| | | | ctgaagggctaccaaatcaccaataagtttgaaagctttaagagcatgag |
| | | | cacgcaaaatggcttcattttttacattccggcgtggctgaccagcaaga |
| | | | tcgatccgtcaaccggctttgttaatttactgaagacgaagtatacctct |
| | | | attgccgactctaaaaaatttattagttccttcgatagaatcatgtatgt |
| | | | tccggaagaggacctgttcgaatttgcactggactacaagaactttagcc |
| | | | gtaccgatgctgattatataaagaaatggaaactctacagctatggcaac |
| | | | cgcatccgtatctttcgcaacccgaaaaaaaacaacgtgttcgactggga |
| | | | agaggtatgtctgacttcagcgtacaaggagcttttcaataaatatggta |
| | | | tcaactaccagcagggtgatatccgtgcactgctttgcgagcaaagcgat |
| | | | aaagcgttctattcaagcttcatggcgctgatgagcctgatgctgcaaat |
| | | | gcgcaacagcatcaccggtcgtaccgatgttgcatttctgatttctccag |
| | | | taaaaaacagcgatggtatcttttacgacagccgtaattacgaggcccaa |
| | | | gaaaatgcgattttgccgaagaacgctgacgcgaatggtgcatacaacat |
| | | | tgcaaggaaagttctgtgggcaatcggccaatttaaaaaagcggaagatg |
| | | | agaagctggacaaagtgaaaattgcgattagcaacaaggagtggctggag |
| | | | tatgcgcagacctccgttaaatcagggggaagtggcggaagcggtgggtc |
| | | | tacgaacctgagcgacattattgaaaaagaaactggtaaacagttggtga |
| | | | tccaagaatccattctgatgttaccggaggaagtggaagaggtgatcggc |
| | | | aacaagccggagtccgacatcctggttcacaccgcgtatgatgagtcgac |
| | | | cgatgagaatgttatgctgctgaccagcgacgccccagagtacaaaccgt |
| | | | gggcgctcgtcatccaggatagcaatggcgaaaacaagatcaagatgttg |
| | | | tctggcggtagcatggctccgaaaaagaagcgtaaagtaggtattcatgg |
| | | | tgttccggcatcaggttaa |
| 55 | Base editor | RrA3F-F1301-dCas9 | atgggaagtcatcaccaccaccaccattcaggtgagaacctgtacttcca |
| | | | gagcatggctccgaaaaagaaacgtaaggtgggtatccacggcgttccgg |
| | | | cgatgaaacctcagattcgcgatcatcgtccgaatcctatggaggcgatg |
| | | | tatccgcatattttctactttcattttgaaaacttagaaaaagcctatgg |
| | | | ccgtaacgaaacttggctgtgctttaccgtagagattatcaaacaatatt |
| | | | taccggtaccctggaaaaaaggtgttttccgtaatcaggttgatccggag |
| | | | acacattgtcatgcagagaagtgcttcctgtcctggttctgcaataacac |
| | | | ccttagcccgaaaaagaattaccaagttacttggtacacgagctggtcgc |
| | | | catgtccagagtgcgcaggagaagtagcagaattcttagctgaacattcg |
| | | | aacgtaaaattgacaatctacaccgcccgtctctactacctgtgggacac |
| | | | cgactatcaggaaggcctgcgctcattgtctgaggagggcgcatcggtcg |
| | | | aaatcatggattatgaagattttcagtattgttgggaaaatttcgtatac |
| | | | gacgatggtgagccttttaaacgttggaaaggcctgaagtacaactttca |
| | | | gtctttgacccgtcgcttgcgcgaaattctgcaatctggaagtgagacac |
| | | | ccgggacttcagaatccgcgaccccggagtctggaatggataaaaagtat |
| | | | tcaataggactagctattggcaccaacagcgttggttgggcagttatcac |
| | | | cgatgaatataaggtcccgagcaagaagtttaaggtcctgggcaacaccg |
| | | | atcgccacagcataaaaaagaaccttattggtgcgctgctgttcgattct |
| | | | ggtgagactgccgaagcgacccgtctgaagagaaccgcgcgtcgtagata |
| | | | cacccggcgcaagaaccgtatctgctatctgcaagaaattttcagcaacg |
| | | | agatggctaaggtcgacgactccttctttcatcgcctggaggaaagcttt |
| | | | ctggttgaggaagacaaaaagcacgagcgtcatccgattttcggtaacat |
| | | | tgttgatgaagttgcctaccacgaaaaataccccacaatttatcatctgc |
| | | | gtaaaaaactggtagactccaccgataaggcggatttacgccttatctat |
| | | | ttggcgctggcgcatatgattaagttccgcggtcattttttgatcgaggg |
| | | | cgacctgaatccggataacagcgacgttgacaaactgtttatccagctgg |
| | | | tgcagacctataaccagttgttcgaagagaacccgattaatgcgtctggc |
| | | | gtggacgcgaaggcgatcctgagcgctcgtctgtccaagtcacgccgcct |
| | | | ggagaacttgatcgcgcaactgccgggtgagaagaaaaacggcttgttcg |
| | | | gcaatctgattgctctgtccctaggtttgaccccgaatttcaaatctaat |
| | | | tttgatctggcggaagatgcgaaattacagttgagcaaggacacttacga |
| | | | tgacgatctggacaacctgctcgctcagatcggcgatcagtatgccgatc |
| | | | ttttcctggccgcgaagaacctgagcgatgccatcttgctatcggacatc |
| | | | ttacgtgttaacaccgaaatcaccaaagctccgctaagcgcgtccatgat |
| | | | taaacgttacgatgaacaccaccaggatctgacgctgctgaaagcgttgg |
| | | | tgcgccagcaactgccggaaaaatacaaggaaattttcttcgaccaaagc |
| | | | aagaacggttatgcaggctatatcgacggtggtgcgtcgcaagaagaatt |
| | | | ttataaattcatcaaaccgattttggagaagatggatggtaccgaggagc |
| | | | tgctggtaaaattgaatcgggaagacctcttgcgaaagcagcgtactttc |
| | | | gataatggtagcatcccgcaccagattcatctgggcgaacttcacgcaat |
| | | | cctacgtcgtcaagaggatttctacccgtttctgaaggataaccgtgaaa |
| | | | agatcgagaagattctgacctttcgtattccgtattatgtgggtccgctg |
| | | | gcgagaggcaacagccgttttgcgtggatgacccgcaagtcggaagagac |
| | | | tatcacgccgtggaatttcgaggaggtcgtggacaagggtgcatcagcgc |
| | | | aatcttttattgaacgtatgaccaattttgataaaaaccttccaaatgaa |
| | | | aaagtgttgccgaagcacagcttgctgtacgagtatttcaccgtttacaa |
| | | | tgaactgacgaaagtcaagtacgttaccgaaggtatgcgtaagccagcct |
| | | | ttctgtctggggagcaaaaaaaagccattgtggatctgctgttcaagacc |
| | | | aatcgcaaggttaccgtcaagcaactgaaagaggattacttcaaaaaaat |
| | | | cgaatgttttgactccgtggaaatcagcggcgttgaagaccgctttaatg |
| | | | catctctgggcacctaccacgatctactcaagatcatcaaggataaggac |
| | | | ttcctggacaacgaagaaaatgaggatatcttggaagatattgtgctgac |
| | | | tttaacgttgttcgaggatcgtgagatgattgaagaacgcctcaaaacgt |
| | | | acgcacatctgtttgacgataaggtgatgaaacagctaaagcgccgtcgt |
| | | | tacaccggttggggtcgtctgtctagaaagctgattaacggcattcgtga |
| | | | caaacaatctggcaagaccatcctggacttcctcaaatccgacggcttcg |
| | | | caaatcgtaatttcatgcaactgatccatgatgatagcctgacctttaaa |
| | | | gaagatattcagaaggctcaagtgtccggccagggtgactcgctgcatga |
| | | | gcatatcgcgaatctggctggctcccctgcgataaaaaaaggtattctcc |
| | | | aaacggtgaaagttgtggatgaactggttaaagtcatgggtcgtcataaa |
| | | | ccggaaaacatcgtgattgagatggcacgtgagaaccagaccacccagaa |
| | | | aggtcaaaaaaacagtcgtgagcgtatgaaacgtatcgaagaggggatta |
| | | | aggagttaggcagccagattctgaaggagcacccggttgagaacacccag |
| | | | ctgcaaaacgagaagctctacctgtattacctgcaaaacggccgtgacat |
| | | | gtatgttgaccaggagctggacatcaatcgtctcagcgactacgacgtgg |
| | | | atgctatcgtgccgcagagctttctaaaagatgattccatcgacaataaa |
| | | | gtgctcacccgtagcgacaagaaccgtggtaaaagcgacaacgtgccgag |
| | | | cgaggaagtagttaagaaaatgaaaaactactggcgtcagctattgaacg |
| | | | ccaagcttatcacccagcgtaagtttgacaacttgacgaaggcagagcgt |
| | | | ggcggcctgagcgagctagacaaggcgggttttattaaacgccagctggt |
| | | | ggaaactcgccagattaccaaacacgttgcgcaaatcttggactcacgca |
| | | | tgaatacgaagtacgacgaaaacgataaattgatccgcgaggtgaaggtc |
| | | | ataaccctgaaatctaaactggttagcgatttccggaaggactttcaatt |
| | | | ttataaagtccgggagattaacaactatcaccatgcacacgacgcttact |
| | | | taaatgcggttgttggcacggccctgatcaagaagtacccgaaactggaa |
| | | | agcgagtttgtttatggtgactacaaggtgtatgacgttcgcaagatgat |
| | | | tgcaaaatccgaacaagagattggtaaggcgacggctaagtattttttct |
| | | | acagcaatatcatgaacttcttcaagactgaaatcaccctggcgaacggt |
| | | | gaaattcgtaaacgtccgttgatcgaaaccaacggtgaaaccggtgaaat |
| | | | cgtttgggataaaggtagggattttgcgaccgttcgtaaggtcctgagca |
| | | | tgccgcaggttaacatcgtgaaaaagaccgaagtgcagaccggcggtttc |
| | | | tccaaggagagtatcttaccgaagcgcaacagtgacaagctgatcgcgcg |
| | | | taaaaaagactgggatccgaaaaaatacggcggctttgactccccgaccg |
| | | | tcgcgtacagcgtactggtcgtggctaaagtggagaaaggcaaatcgaaa |
| | | | aagctgaaatccgttaaagagttgctgggtattacgattatggaacgttc |
| | | | gagcttcgagaaaaatccgatcgacttcctggaggccaaaggctacaagg |
| | | | aagttaaaaaagacctgatcattaaactgccgaagtacagcctgttcgag |
| | | | ctcgagaacggtcgcaaacgcatgctggcgagcgcgggtgagctccaaaa |
| | | | aggcaacgaattggctctgccgagcaagtacgtgaacttcctgtatctgg |
| | | | cctcccattatgaaaaactgaagggtagcccggaagataacgaacaaaag |
| | | | cagctgttcgtggaacaacataaacactatctggacgagattattgagca |
| | | | aatcagcgagttcagcaaacgcgtgattctggcggatgctaatctggaca |
| | | | aggttctttcagcctataataaacaccgtgataagccgatccgtgaacag |
| | | | gcagaaaatattattcacctgttcaccctgacgaacctgggtgcgccagc |
| | | | agcgtttaaatacttcgacacaacgatcgaccggaagcgttataccagca |
| | | | ccaaagaggttcttgacgccaccttgattcaccaaagcatcacgggtctg |
| | | | tatgaaacccgtattgacttgagccagttgggcggagattcagggggaag |
| | | | tggcggaagcggtgggtctacgaacctgagcgacattattgaaaaagaaa |
| | | | ctggtaaacagttggtgatccaagaatccattctgatgttaccggaggaa |
| | | | gtggaagaggtgatcggcaacaagccggagtccgacatcctggttcacac |
| | | | cgcgtatgatgagtcgaccgatgagaatgttatgctgctgaccagcgacg |
| | | | ccccagagtacaaaccgtgggcgctcgtcatccaggatagcaatggcgaa |
| | | | aacaagatcaagatgttgtctggcggtagcatggctccgaaaaagaagcg |
| | | | taaagtaggtattcatggtgttccggcatcaggttaa |
| 56 | Base editor | RrA3F-F1301- | atgggaagtcatcaccaccaccaccattcaggtgagaacctgtacttcca |
| | | dCasPhi-2 | gagcatggctccgaaaaagaaacgtaaggtgggtatccacggcgttccgg |
| | | | cgatgaaacctcagattcgcgatcatcgtccgaatcctatggaggcgatg |
| | | | tatccgcatattttctactttcattttgaaaacttagaaaaagcctatgg |
| | | | ccgtaacgaaacttggctgtgctttaccgtagagattatcaaacaatatt |
| | | | taccggtaccctggaaaaaaggtgttttccgtaatcaggttgatccggag |
| | | | acacattgtcatgcagagaagtgcttcctgtcctggttctgcaataacac |
| | | | ccttagcccgaaaaagaattaccaagttacttggtacacgagctggtcgc |
| | | | catgtccagagtgcgcaggagaagtagcagaattcttagctgaacattcg |
| | | | aacgtaaaattgacaatctacaccgcccgtctctactacctgtgggacac |
| | | | cgactatcaggaaggcctgcgctcattgtctgaggagggcgcatcggtcg |
| | | | aaatcatggattatgaagattttcagtattgttgggaaaatttcgtatac |
| | | | gacgatggtgagccttttaaacgttggaaaggcctgaagtacaactttca |
| | | | gtctttgacccgtcgcttgcgcgaaattctgcaatctggaagtgagacac |
| | | | ccgggacttcagaatccgcgaccccggagtctggaatgccaaaacccgct |
| | | | gtagagtcagaattttcaaaagttttgaaaaagcactttccgggagaacg |
| | | | tttccgctcgagctatatgaaaagaggcggtaagatcctggctgcccaag |
| | | | gcgaagaggcagttgttgcgtatctacagggcaagtccgaggaggagcct |
| | | | ccgaacttccagccaccggcgaagtgccacgtggtgaccaaatcccgtga |
| | | | cttcgcggaatggccgatcatgaaggcgtcggaagcgattcaacgttata |
| | | | tctatgcactgtcgaccaccgaacgcgcggcctgtaaaccgggcaaaagt |
| | | | agtgagagccatgcagcgtggttcgccgctaccggtgtcagcaaccacgg |
| | | | ctacagccacgttcagggtctgaacctgatttttgatcataccttgggcc |
| | | | gttacgatggtgttctgaaaaaggtccaactgcgcaacgaaaaggcgaga |
| | | | gcgcgtttagagagcattaacgcgtctagagcagatgaaggtttaccgga |
| | | | gattaaagccgaggaggaagaagtggctacgaacgaaaccggccacctgt |
| | | | tgcaaccgccgggtatcaatccgagcttttacgtgtatcagaccattagc |
| | | | ccgcaggcataccgtccgcgtgacgagatcgtgctgccgccggagtacgc |
| | | | aggttacgtacgtgacccgaatgctccgattccgctgggtgtggtgcgta |
| | | | accgttgcgatatccaaaaagggtgcccgggctacatcccggaatggcag |
| | | | cgtgaagcaggtaccgcgatcagcccgaaaacaggcaaggccgtaactgt |
| | | | gccgggcctgtctccgaagaaaaacaaacgtatgcgtaggtactggcgct |
| | | | ctgagaaggagaaagctcaagatgcgctgttggttaccgtacgcattggt |
| | | | accgattgggttgtcatcgacgtgcgcggtctgttgcgcaacgcccgttg |
| | | | gcgcaccatcgctcccaaggacatcagcctgaatgcgctcctcgatttgt |
| | | | ttaccggtgatccggtgatcgatgtgcgtcgcaatattgttacctttacg |
| | | | tacaccctcgacgcctgtggtacctacgcccgtaaatggaccttgaaagg |
| | | | caaacagacgaaagcgaccctggacaaactgacggccactcagacggttg |
| | | | cgttggttgcgattgcgcttggtcagaccaacccgatttccgctggcatt |
| | | | agccgtgttacacaagaaaatggtgcactgcaatgcgaaccgctggaccg |
| | | | gttcaccctgccggatgacctgttgaaggacatcagcgcatatcgcattg |
| | | | cttgggacagaaacgaagaggaactacgcgctcgcagcgtcgaggccctg |
| | | | ccggaggcgcaacaagcggaggtccgtgcgctggacggggtgtccaaaga |
| | | | aaccgcgcgtacccaactgtgcgcagatttcggcctggacccgaaacgtc |
| | | | tgccgtgggataaaatgagctccaatactacctttatcagcgaagccttg |
| | | | ctgtctaacagcgtgagccgtgatcaggtttttttcactccggctccgaa |
| | | | gaagggtgctaagaaaaaagcgcctgttgaagttatgcgtaaagaccgca |
| | | | cttgggcacgtgcgtacaagccgcgtctgagcgtcgaagctcagaaactg |
| | | | aagaacgaagccctgtgggcacttaagcgcacctccccggagtatttgaa |
| | | | gctgagccgtcgcaaagaggagctgtgtcgtcgtagcatcaactatgtta |
| | | | tcgaaaagacccgtcgccgtacgcaatgtcagattgtgatcccggtgatc |
| | | | gaggatctgaatgttcgcttctttcatggttctggtaagagactgcccgg |
| | | | ttgggataatttcttcaccgctaaaaaagagaaccgttggttcatccagg |
| | | | gcctgcacaaagcgtttagcgatctgcgtacgcaccgttccttctatgtt |
| | | | tttgaagttcgtccggagcgcacctctattacctgtccaaagtgcggtca |
| | | | ttgcgaggtgggtaatcgtgacggtgaggcgtttcagtgcctgagctgcg |
| | | | gcaagacgtgcaacgcagatcttgacgtcgcgacccataatctgacccaa |
| | | | gtggcgttgaccggtaagaccatgccgaagcgcgaggagccgcgtgacgc |
| | | | tcagggtacagcgccagcgcgcaagactaagaaggcgagcaaatcaaagg |
| | | | cgccaccggcggagcgcgaagaccagaccccggcgcaagagccaagccag |
| | | | accagttctggaggtagcgggggtagcggtggtagcactaacttaagtga |
| | | | tattatcgagaaagaaaccgggaaacagctggtgatccaagaaagcattc |
| | | | tgatgctgcccgaagaagttgaagaagtgattggtaacaagcctgagtcg |
| | | | gacatcttagtgcacaccgcgtacgatgaaagcacggatgagaatgtgat |
| | | | gctgctcacctctgatgcccccgaatacaagccgtgggctctggtgatcc |
| | | | aggactctaacggagaaaacaaaattaagatgctctctggcggctccatg |
| | | | gcgcctaaaaaaaaacgcaaagtggggattcacggggttccggcgtaa |
| 57 | Base editor | RrA3F-F1301- | atgggaagtcatcaccaccaccaccattcaggtgagaacctgtacttcca |
| | | dLbCas12a | gagcatggctccgaaaaagaaacgtaaggtgggtatccacggcgttccgg |
| | | | cgatgaaacctcagattcgcgatcatcgtccgaatcctatggaggcgatg |
| | | | tatccgcatattttctactttcattttgaaaacttagaaaaagcctatgg |
| | | | ccgtaacgaaacttggctgtgctttaccgtagagattatcaaacaatatt |
| | | | taccggtaccctggaaaaaaggtgttttccgtaatcaggttgatccggag |
| | | | acacattgtcatgcagagaagtgcttcctgtcctggttctgcaataacac |
| | | | ccttagcccgaaaaagaattaccaagttacttggtacacgagctggtcgc |
| | | | catgtccagagtgcgcaggagaagtagcagaattcttagctgaacattcg |
| | | | aacgtaaaattgacaatctacaccgcccgtctctactacctgtgggacac |
| | | | cgactatcaggaaggcctgcgctcattgtctgaggagggcgcatcggtcg |
| | | | aaatcatggattatgaagattttcagtattgttgggaaaatttcgtatac |
| | | | gacgatggtgagccttttaaacgttggaaaggcctgaagtacaactttca |
| | | | gtctttgacccgtcgcttgcgcgaaattctgcaatctggaagtgagacac |
| | | | ccgggacttcagaatccgcgaccccggagtctggaatgtcaaaactagaa |
| | | | aagtttacaaattgttactccctgtcgaaaaccctgagattcaaagcgat |
| | | | cccggtgggtaaaactcaagagaacattgataacaaacgtctgctggttg |
| | | | aggacgaaaagcgcgcggaggattacaaaggcgttaaaaagctgttggac |
| | | | cgttattacctgtcctttattaatgatgttctgcacagcattaaattaaa |
| | | | gaacctaaacaattacatctccctcttccgtaaaaagacccgcaccgaaa |
| | | | aggaaaataaagaactggagaaccttgagattaatctgcgtaaagaaatc |
| | | | gctaaagcgtttaaaggcaacgagggttataaatccttatttaaaaaaga |
| | | | catcatcgaaacgattctcccagaattcctggacgacaaagacgagatcg |
| | | | cgctggttaattccttcaacggctttaccaccgcattcaccggctttttt |
| | | | gataaccgcgaaaacatgttctctgaagaggccaaaagcacctcaattgc |
| | | | gtttcgctgcattaacgaaaatctgacgcgttatatctctaacatggata |
| | | | tcttcgagaaggttgacgctatctttgataaacatgaagtccaggagatc |
| | | | aaggagaagatcctgaacagcgactacgacgtggaggatttcttcgaggg |
| | | | tgagttcttcaattttgttttgacccaagaaggcatcgatgtctacaatg |
| | | | cgataattggtggttttgttaccgagagcggggagaaaattaagggtctg |
| | | | aacgaatatatcaacttgtacaaccagaaaaccaaacaaaaactgccgaa |
| | | | gtttaagccgctgtacaagcaagtcttgtcggatcgtgaatcgctgagct |
| | | | tttacggcgaaggctacaccagcgatgaggaggtgttggaggtgttccgc |
| | | | aatacgttgaataagaactctgaaattttttccagcattaagaagctgga |
| | | | aaaactattcaagaacttcgacgagtatagcagcgcaggtatctttgtga |
| | | | aaaacggtccggctattagcaccatctcgaaagacattttcggcgaatgg |
| | | | aatgttattcgtgataagtggaatgccgaatacgacgacattcatctgaa |
| | | | gaaaaaggcggttgtaactgaaaagtacgaggacgatcgtcgtaagtcgt |
| | | | tcaaaaagatcggctccttctctctggagcaactgcaagagtacgccgat |
| | | | gccgatttgtcagtcgtggagaagctgaaagagatcatcattcaaaaagt |
| | | | tgacgaaatctataaggtctatggtagcagtgaaaaactgtttgacgcgg |
| | | | acttcgtgctggagaaaagcctgaagaaaaatgacgccgtggtggcaatc |
| | | | atgaaagatcttctggatagcgtcaagtctttcgaaaactacatcaaagc |
| | | | gtttttcggtgaaggcaaggaaacgaatcgtgacgagagcttctatggtg |
| | | | atttcgtcctagcctatgatatcctgcttaaggtggatcacatctatgat |
| | | | gccatccgcaactatgttacccagaaaccgtactcaaaggacaagttcaa |
| | | | gttatacttccagaatccgcagtttatgggtggctgggataaagataagg |
| | | | aaaccgactatcgcgccaccattttacgttacggtagtaagtactatctg |
| | | | gcaatcatggataaaaaatatgcgaaatgcctgcaaaagatcgacaagga |
| | | | cgatgttaacggcaattatgagaagatcaactacaaactgctgccgggcc |
| | | | caaataagatgctgccgaaggtgtttttcagcaaaaaatggatggcgtat |
| | | | tacaatccgagcgaggacatccaaaagatctacaaaaacggtacgtttaa |
| | | | gaagggtgatatgttcaacctcaacgattgtcataaacttattgacttct |
| | | | tcaaggatagcatatctcgttatccgaagtggagcaacgcgtatgacttt |
| | | | aactttagcgaaaccgagaaatacaaagatatcgcgggtttctatcgtga |
| | | | agttgaagaacagggttacaaagtgtccttcgagtccgcgagcaagaagg |
| | | | aggtagacaagctggtcgaggagggtaagttgtacatgtttcagatttac |
| | | | aataaggacttttccgacaagtcccatggtacaccgaatttgcacaccat |
| | | | gtatttcaagcttctgttcgacgagaacaaccacggccagatccgtctga |
| | | | gcggtggtgcagagctgttcatgcgtagagctagcctgaagaaagaagag |
| | | | ttggtcgttcatccggctaattcgccgatcgcgaacaaaaatccagataa |
| | | | ccctaaaaagactacgaccctgagctatgatgtgtataaagacaaacgct |
| | | | ttagcgaagaccagtatgagttacacattccgatcgctatcaacaagtgc |
| | | | ccgaagaatattttcaagatcaacaccgaagtgcgtgttcttttgaagca |
| | | | cgatgacaacccgtatgtgattggtattgctcgtggtgaacgtaatctgc |
| | | | tgtacattgtggttgtggacggcaaaggaaacatcgtggaacagtacagc |
| | | | ctgaacgaaatcattaacaatttcaacggcattcgcattaagacggatta |
| | | | ccattctttgctggacaaaaaggagaaagagcgtttcgaggcccgtcaga |
| | | | actggacctccatcgagaacattaaggagctgaaggcgggttacatcagc |
| | | | caggtggtgcacaaaatctgcgaactggtggagaagtacgacgcggtgat |
| | | | tgctttggcggatttgaactccggttttaagaacagccgcgttaaagtgg |
| | | | aaaagcaggtttatcagaaattcgaaaaaatgctgattgataaattgaat |
| | | | tacatggttgacaaaaagtctaacccgtgtgcaaccggcggtgcgctgaa |
| | | | gggctaccaaatcaccaataagtttgaaagctttaagagcatgagcacgc |
| | | | aaaatggcttcattttttacattccggcgtggctgaccagcaagatcgat |
| | | | ccgtcaaccggctttgttaatttactgaagacgaagtatacctctattgc |
| | | | cgactctaaaaaatttattagttccttcgatagaatcatgtatgttccgg |
| | | | aagaggacctgttcgaatttgcactggactacaagaactttagccgtacc |
| | | | gatgctgattatataaagaaatggaaactctacagctatggcaaccgcat |
| | | | ccgtatctttcgcaacccgaaaaaaaacaacgtgttcgactgggaagagg |
| | | | tatgtctgacttcagcgtacaaggagcttttcaataaatatggtatcaac |
| | | | taccagcagggtgatatccgtgcactgctttgcgagcaaagcgataaagc |
| | | | gttctattcaagcttcatggcgctgatgagcctgatgctgcaaatgcgca |
| | | | acagcatcaccggtcgtaccgatgttgcatttctgatttctccagtaaaa |
| | | | aacagcgatggtatcttttacgacagccgtaattacgaggcccaagaaaa |
| | | | tgcgattttgccgaagaacgctgacgcgaatggtgcatacaacattgcaa |
| | | | ggaaagttctgtgggcaatcggccaatttaaaaaagcggaagatgagaag |
| | | | ctggacaaagtgaaaattgcgattagcaacaaggagtggctggagtatgc |
| | | | gcagacctccgttaaatcagggggaagtggcggaagcggtgggtctacga |
| | | | acctgagcgacattattgaaaaagaaactggtaaacagttggtgatccaa |
| | | | gaatccattctgatgttaccggaggaagtggaagaggtgatcggcaacaa |
| | | | gccggagtccgacatcctggttcacaccgcgtatgatgagtcgaccgatg |
| | | | agaatgttatgctgctgaccagcgacgccccagagtacaaaccgtgggcg |
| | | | ctcgtcatccaggatagcaatggcgaaaacaagatcaagatgttgtctgg |
| | | | cggtagcatggctccgaaaaagaagcgtaaagtaggtattcatggtgttc |
| | | | cggcatcaggttaa |
| 58 | Base editor | SsA3B-R54Q-dCas9 | atgggaagtcatcaccaccaccaccattcaggtgagaacctgtacttcca |
| | | | gagcatggctccgaaaaagaaacgtaaggtgggtatccacggcgttccgg |
| | | | cgatggatccacagaggctaagacaatggcccggtccgggtccggcgagc |
| | | | cgcggtggctacggccagcgtccgcgtatccgcaatccggaagagtggtt |
| | | | ccacgaactgagtccgagaacctttagcttccactttcgtaacctgcgtt |
| | | | ttgctagcggtcagaatcgcagctatatctgctgccaggtggaaggcaag |
| | | | aactgcttcttccaaggtatttttcaaaaccaggttccgccggacccacc |
| | | | ttgtcatgctgaactttgctttttgtcctggttccaaagctggggtttgt |
| | | | cgccggacgagcactattacgtgacctggttcatctcctggtccccgtgc |
| | | | tgtgaatgtgcggcgaaagtcgcccaatttctggaagagaaccgcaacgt |
| | | | gagcttgtctttgtctgcggcgcgtctgtactacttctggaaaagcgagt |
| | | | cccgtgagggcctgcgccgtctgtccgatctgggtgcacaggttggtatt |
| | | | atgtcgttccaagattttcagcattgttggaataactttgttcataatct |
| | | | gggcatgccgttccagccgtggaagaaattgcacaaaaactaccaacgtc |
| | | | tggtgaccgaattaaaacagattctccgcgaggagccggctacctatggc |
| | | | tctccacaggcgcaaggtaaggtgcgcatcggcagcaccgcggcgggtct |
| | | | gcgtcacagccatagccacacccgtagcgaggcgcatctgcgtcccaacc |
| | | | actcatctcgtcagcaccgtatcctgaatccgccgcgcgaggcccgtgcg |
| | | | cgtacgtgcgttctggtagacgcatcctggatttgctatcgttctggaag |
| | | | tgagacacccgggacttcagaatccgcgaccccggagtctggaatggata |
| | | | aaaagtattcaataggactagctattggcaccaacagcgttggttgggca |
| | | | gttatcaccgatgaatataaggtcccgagcaagaagtttaaggtcctggg |
| | | | caacaccgatcgccacagcataaaaaagaaccttattggtgcgctgctgt |
| | | | tcgattctggtgagactgccgaagcgacccgtctgaagagaaccgcgcgt |
| | | | cgtagatacacccggcgcaagaaccgtatctgctatctgcaagaaatttt |
| | | | cagcaacgagatggctaaggtcgacgactccttctttcatcgcctggagg |
| | | | aaagctttctggttgaggaagacaaaaagcacgagcgtcatccgattttc |
| | | | ggtaacattgttgatgaagttgcctaccacgaaaaataccccacaattta |
| | | | tcatctgcgtaaaaaactggtagactccaccgataaggcggatttacgcc |
| | | | ttatctatttggcgctggcgcatatgattaagttccgcggtcattttttg |
| | | | atcgagggcgacctgaatccggataacagcgacgttgacaaactgtttat |
| | | | ccagctggtgcagacctataaccagttgttcgaagagaacccgattaatg |
| | | | cgtctggcgtggacgcgaaggcgatcctgagcgctcgtctgtccaagtca |
| | | | cgccgcctggagaacttgatcgcgcaactgccgggtgagaagaaaaacgg |
| | | | cttgttcggcaatctgattgctctgtccctaggtttgaccccgaatttca |
| | | | aatctaattttgatctggcggaagatgcgaaattacagttgagcaaggac |
| | | | acttacgatgacgatctggacaacctgctcgctcagatcggcgatcagta |
| | | | tgccgatcttttcctggccgcgaagaacctgagcgatgccatcttgctat |
| | | | cggacatcttacgtgttaacaccgaaatcaccaaagctccgctaagcgcg |
| | | | tccatgattaaacgttacgatgaacaccaccaggatctgacgctgctgaa |
| | | | agcgttggtgcgccagcaactgccggaaaaatacaaggaaattttcttcg |
| | | | accaaagcaagaacggttatgcaggctatatcgacggtggtgcgtcgcaa |
| | | | gaagaattttataaattcatcaaaccgattttggagaagatggatggtac |
| | | | cgaggagctgctggtaaaattgaatcgggaagacctcttgcgaaagcagc |
| | | | gtactttcgataatggtagcatcccgcaccagattcatctgggcgaactt |
| | | | cacgcaatcctacgtcgtcaagaggatttctacccgtttctgaaggataa |
| | | | ccgtgaaaagatcgagaagattctgacctttcgtattccgtattatgtgg |
| | | | gtccgctggcgagaggcaacagccgttttgcgtggatgacccgcaagtcg |
| | | | gaagagactatcacgccgtggaatttcgaggaggtcgtggacaagggtgc |
| | | | atcagcgcaatcttttattgaacgtatgaccaattttgataaaaaccttc |
| | | | caaatgaaaaagtgttgccgaagcacagcttgctgtacgagtatttcacc |
| | | | gtttacaatgaactgacgaaagtcaagtacgttaccgaaggtatgcgtaa |
| | | | gccagcctttctgtctggggagcaaaaaaaagccattgtggatctgctgt |
| | | | tcaagaccaatcgcaaggttaccgtcaagcaactgaaagaggattacttc |
| | | | aaaaaaatcgaatgttttgactccgtggaaatcagcggcgttgaagaccg |
| | | | ctttaatgcatctctgggcacctaccacgatctactcaagatcatcaagg |
| | | | ataaggacttcctggacaacgaagaaaatgaggatatcttggaagatatt |
| | | | gtgctgactttaacgttgttcgaggatcgtgagatgattgaagaacgcct |
| | | | caaaacgtacgcacatctgtttgacgataaggtgatgaaacagctaaagc |
| | | | gccgtcgttacaccggttggggtcgtctgtctagaaagctgattaacggc |
| | | | attcgtgacaaacaatctggcaagaccatcctggacttcctcaaatccga |
| | | | cggcttcgcaaatcgtaatttcatgcaactgatccatgatgatagcctga |
| | | | cctttaaagaagatattcagaaggctcaagtgtccggccagggtgactcg |
| | | | ctgcatgagcatatcgcgaatctggctggctcccctgcgataaaaaaagg |
| | | | tattctccaaacggtgaaagttgtggatgaactggttaaagtcatgggtc |
| | | | gtcataaaccggaaaacatcgtgattgagatggcacgtgagaaccagacc |
| | | | acccagaaaggtcaaaaaaacagtcgtgagcgtatgaaacgtatcgaaga |
| | | | ggggattaaggagttaggcagccagattctgaaggagcacccggttgaga |
| | | | acacccagctgcaaaacgagaagctctacctgtattacctgcaaaacggc |
| | | | cgtgacatgtatgttgaccaggagctggacatcaatcgtctcagcgacta |
| | | | cgacgtggatgctatcgtgccgcagagctttctaaaagatgattccatcg |
| | | | acaataaagtgctcacccgtagcgacaagaaccgtggtaaaagcgacaac |
| | | | gtgccgagcgaggaagtagttaagaaaatgaaaaactactggcgtcagct |
| | | | attgaacgccaagcttatcacccagcgtaagtttgacaacttgacgaagg |
| | | | cagagcgtggcggcctgagcgagctagacaaggcgggttttattaaacgc |
| | | | cagctggtggaaactcgccagattaccaaacacgttgcgcaaatcttgga |
| | | | ctcacgcatgaatacgaagtacgacgaaaacgataaattgatccgcgagg |
| | | | tgaaggtcataaccctgaaatctaaactggttagcgatttccggaaggac |
| | | | tttcaattttataaagtccgggagattaacaactatcaccatgcacacga |
| | | | cgcttacttaaatgcggttgttggcacggccctgatcaagaagtacccga |
| | | | aactggaaagcgagtttgtttatggtgactacaaggtgtatgacgttcgc |
| | | | aagatgattgcaaaatccgaacaagagattggtaaggcgacggctaagta |
| | | | ttttttctacagcaatatcatgaacttcttcaagactgaaatcaccctgg |
| | | | cgaacggtgaaattcgtaaacgtccgttgatcgaaaccaacggtgaaacc |
| | | | ggtgaaatcgtttgggataaaggtagggattttgcgaccgttcgtaaggt |
| | | | cctgagcatgccgcaggttaacatcgtgaaaaagaccgaagtgcagaccg |
| | | | gcggtttctccaaggagagtatcttaccgaagcgcaacagtgacaagctg |
| | | | atcgcgcgtaaaaaagactgggatccgaaaaaatacggcggctttgactc |
| | | | cccgaccgtcgcgtacagcgtactggtcgtggctaaagtggagaaaggca |
| | | | aatcgaaaaagctgaaatccgttaaagagttgctgggtattacgattatg |
| | | | gaacgttcgagcttcgagaaaaatccgatcgacttcctggaggccaaagg |
| | | | ctacaaggaagttaaaaaagacctgatcattaaactgccgaagtacagcc |
| | | | tgttcgagctcgagaacggtcgcaaacgcatgctggcgagcgcgggtgag |
| | | | ctccaaaaaggcaacgaattggctctgccgagcaagtacgtgaacttcct |
| | | | gtatctggcctcccattatgaaaaactgaagggtagcccggaagataacg |
| | | | aacaaaagcagctgttcgtggaacaacataaacactatctggacgagatt |
| | | | attgagcaaatcagcgagttcagcaaacgcgtgattctggcggatgctaa |
| | | | tctggacaaggttctttcagcctataataaacaccgtgataagccgatcc |
| | | | gtgaacaggcagaaaatattattcacctgttcaccctgacgaacctgggt |
| | | | gcgccagcagcgtttaaatacttcgacacaacgatcgaccggaagcgtta |
| | | | taccagcaccaaagaggttcttgacgccaccttgattcaccaaagcatca |
| | | | cgggtctgtatgaaacccgtattgacttgagccagttgggcggagattca |
| | | | gggggaagtggcggaagcggtgggtctacgaacctgagcgacattattga |
| | | | aaaagaaactggtaaacagttggtgatccaagaatccattctgatgttac |
| | | | cggaggaagtggaagaggtgatcggcaacaagccggagtccgacatcctg |
| | | | gttcacaccgcgtatgatgagtcgaccgatgagaatgttatgctgctgac |
| | | | cagcgacgccccagagtacaaaccgtgggcgctcgtcatccaggatagca |
| | | | atggcgaaaacaagatcaagatgttgtctggcggtagcatggctccgaaa |
| | | | aagaagcgtaaagtaggtattcatggtgttccggcatcaggttaa |
| 59 | Base editor | SsA3B-R54Q- | atgggaagtcatcaccaccaccaccattcaggtgagaacctgtacttcca |
| | | dCasPhi-2 | gagcatggctccgaaaaagaaacgtaaggtgggtatccacggcgttccgg |
| | | | cgatggatccacagaggctaagacaatggcccggtccgggtccggcgagc |
| | | | cgcggtggctacggccagcgtccgcgtatccgcaatccggaagagtggtt |
| | | | ccacgaactgagtccgagaacctttagcttccactttcgtaacctgcgtt |
| | | | ttgctagcggtcagaatcgcagctatatctgctgccaggtggaaggcaag |
| | | | aactgcttcttccaaggtatttttcaaaaccaggttccgccggacccacc |
| | | | ttgtcatgctgaactttgctttttgtcctggttccaaagctggggtttgt |
| | | | cgccggacgagcactattacgtgacctggttcatctcctggtccccgtgc |
| | | | tgtgaatgtgcggcgaaagtcgcccaatttctggaagagaaccgcaacgt |
| | | | gagcttgtctttgtctgcggcgcgtctgtactacttctggaaaagcgagt |
| | | | cccgtgagggcctgcgccgtctgtccgatctgggtgcacaggttggtatt |
| | | | atgtcgttccaagattttcagcattgttggaataactttgttcataatct |
| | | | gggcatgccgttccagccgtggaagaaattgcacaaaaactaccaacgtc |
| | | | tggtgaccgaattaaaacagattctccgcgaggagccggctacctatggc |
| | | | tctccacaggcgcaaggtaaggtgcgcatcggcagcaccgcggcgggtct |
| | | | gcgtcacagccatagccacacccgtagcgaggcgcatctgcgtcccaacc |
| | | | actcatctcgtcagcaccgtatcctgaatccgccgcgcgaggcccgtgcg |
| | | | cgtacgtgcgttctggtagacgcatcctggatttgctatcgttctggaag |
| | | | tgagacacccgggacttcagaatccgcgaccccggagtctggaatgccaa |
| | | | aacccgctgtagagtcagaattttcaaaagttttgaaaaagcactttccg |
| | | | ggagaacgtttccgctcgagctatatgaaaagaggcggtaagatcctggc |
| | | | tgcccaaggcgaagaggcagttgttgcgtatctacagggcaagtccgagg |
| | | | aggagcctccgaacttccagccaccggcgaagtgccacgtggtgaccaaa |
| | | | tcccgtgacttcgcggaatggccgatcatgaaggcgtcggaagcgattca |
| | | | acgttatatctatgcactgtcgaccaccgaacgcgcggcctgtaaaccgg |
| | | | gcaaaagtagtgagagccatgcagcgtggttcgccgctaccggtgtcagc |
| | | | aaccacggctacagccacgttcagggtctgaacctgatttttgatcatac |
| | | | cttgggccgttacgatggtgttctgaaaaaggtccaactgcgcaacgaaa |
| | | | aggcgagagcgcgtttagagagcattaacgcgtctagagcagatgaaggt |
| | | | ttaccggagattaaagccgaggaggaagaagtggctacgaacgaaaccgg |
| | | | ccacctgttgcaaccgccgggtatcaatccgagcttttacgtgtatcaga |
| | | | ccattagcccgcaggcataccgtccgcgtgacgagatcgtgctgccgccg |
| | | | gagtacgcaggttacgtacgtgacccgaatgctccgattccgctgggtgt |
| | | | ggtgcgtaaccgttgcgatatccaaaaagggtgcccgggctacatcccgg |
| | | | aatggcagcgtgaagcaggtaccgcgatcagcccgaaaacaggcaaggcc |
| | | | gtaactgtgccgggcctgtctccgaagaaaaacaaacgtatgcgtaggta |
| | | | ctggcgctctgagaaggagaaagctcaagatgcgctgttggttaccgtac |
| | | | gcattggtaccgattgggttgtcatcgacgtgcgcggtctgttgcgcaac |
| | | | gcccgttggcgcaccatcgctcccaaggacatcagcctgaatgcgctcct |
| | | | cgatttgtttaccggtgatccggtgatcgatgtgcgtcgcaatattgtta |
| | | | cctttacgtacaccctcgacgcctgtggtacctacgcccgtaaatggacc |
| | | | ttgaaaggcaaacagacgaaagcgaccctggacaaactgacggccactca |
| | | | gacggttgcgttggttgcgattgcgcttggtcagaccaacccgatttccg |
| | | | ctggcattagccgtgttacacaagaaaatggtgcactgcaatgcgaaccg |
| | | | ctggaccggttcaccctgccggatgacctgttgaaggacatcagcgcata |
| | | | tcgcattgcttgggacagaaacgaagaggaactacgcgctcgcagcgtcg |
| | | | aggccctgccggaggcgcaacaagcggaggtccgtgcgctggacggggtg |
| | | | tccaaagaaaccgcgcgtacccaactgtgcgcagatttcggcctggaccc |
| | | | gaaacgtctgccgtgggataaaatgagctccaatactacctttatcagcg |
| | | | aagccttgctgtctaacagcgtgagccgtgatcaggtttttttcactccg |
| | | | gctccgaagaagggtgctaagaaaaaagcgcctgttgaagttatgcgtaa |
| | | | agaccgcacttgggcacgtgcgtacaagccgcgtctgagcgtcgaagctc |
| | | | agaaactgaagaacgaagccctgtgggcacttaagcgcacctccccggag |
| | | | tatttgaagctgagccgtcgcaaagaggagctgtgtcgtcgtagcatcaa |
| | | | ctatgttatcgaaaagacccgtcgccgtacgcaatgtcagattgtgatcc |
| | | | cggtgatcgaggatctgaatgttcgcttctttcatggttctggtaagaga |
| | | | ctgcccggttgggataatttcttcaccgctaaaaaagagaaccgttggtt |
| | | | catccagggcctgcacaaagcgtttagcgatctgcgtacgcaccgttcct |
| | | | tctatgtttttgaagttcgtccggagcgcacctctattacctgtccaaag |
| | | | tgcggtcattgcgaggtgggtaatcgtgacggtgaggcgtttcagtgcct |
| | | | gagctgcggcaagacgtgcaacgcagatcttgacgtcgcgacccataatc |
| | | | tgacccaagtggcgttgaccggtaagaccatgccgaagcgcgaggagccg |
| | | | cgtgacgctcagggtacagcgccagcgcgcaagactaagaaggcgagcaa |
| | | | atcaaaggcgccaccggcggagcgcgaagaccagaccccggcgcaagagc |
| | | | caagccagaccagttctggaggtagcgggggtagcggtggtagcactaac |
| | | | ttaagtgatattatcgagaaagaaaccgggaaacagctggtgatccaaga |
| | | | aagcattctgatgctgcccgaagaagttgaagaagtgattggtaacaagc |
| | | | ctgagtcggacatcttagtgcacaccgcgtacgatgaaagcacggatgag |
| | | | aatgtgatgctgctcacctctgatgcccccgaatacaagccgtgggctct |
| | | | ggtgatccaggactctaacggagaaaacaaaattaagatgctctctggcg |
| | | | gctccatggcgcctaaaaaaaaacgcaaagtggggattcacggggttccg |
| | | | gcgtaa |
| 60 | Base editor | SsA3B-R54Q- | atgggaagtcatcaccaccaccaccattcaggtgagaacctgtacttcca |
| | | dLbCas12a | gagcatggctccgaaaaagaaacgtaaggtgggtatccacggcgttccgg |
| | | | cgatggatccacagaggctaagacaatggcccggtccgggtccggcgagc |
| | | | cgcggtggctacggccagcgtccgcgtatccgcaatccggaagagtggtt |
| | | | ccacgaactgagtccgagaacctttagcttccactttcgtaacctgcgtt |
| | | | ttgctagcggtcagaatcgcagctatatctgctgccaggtggaaggcaag |
| | | | aactgcttcttccaaggtatttttcaaaaccaggttccgccggacccacc |
| | | | ttgtcatgctgaactttgctttttgtcctggttccaaagctggggtttgt |
| | | | cgccggacgagcactattacgtgacctggttcatctcctggtccccgtgc |
| | | | tgtgaatgtgcggcgaaagtcgcccaatttctggaagagaaccgcaacgt |
| | | | gagcttgtctttgtctgcggcgcgtctgtactacttctggaaaagcgagt |
| | | | cccgtgagggcctgcgccgtctgtccgatctgggtgcacaggttggtatt |
| | | | atgtcgttccaagattttcagcattgttggaataactttgttcataatct |
| | | | gggcatgccgttccagccgtggaagaaattgcacaaaaactaccaacgtc |
| | | | tggtgaccgaattaaaacagattctccgcgaggagccggctacctatggc |
| | | | tctccacaggcgcaaggtaaggtgcgcatcggcagcaccgcggcgggtct |
| | | | gcgtcacagccatagccacacccgtagcgaggcgcatctgcgtcccaacc |
| | | | actcatctcgtcagcaccgtatcctgaatccgccgcgcgaggcccgtgcg |
| | | | cgtacgtgcgttctggtagacgcatcctggatttgctatcgttctggaag |
| | | | tgagacacccgggacttcagaatccgcgaccccggagtctggaatgtcaa |
| | | | aactagaaaagtttacaaattgttactccctgtcgaaaaccctgagattc |
| | | | aaagcgatcccggtgggtaaaactcaagagaacattgataacaaacgtct |
| | | | gctggttgaggacgaaaagcgcgcggaggattacaaaggcgttaaaaagc |
| | | | tgttggaccgttattacctgtcctttattaatgatgttctgcacagcatt |
| | | | aaattaaagaacctaaacaattacatctccctcttccgtaaaaagacccg |
| | | | caccgaaaaggaaaataaagaactggagaaccttgagattaatctgcgta |
| | | | aagaaatcgctaaagcgtttaaaggcaacgagggttataaatccttattt |
| | | | aaaaaagacatcatcgaaacgattctcccagaattcctggacgacaaaga |
| | | | cgagatcgcgctggttaattccttcaacggctttaccaccgcattcaccg |
| | | | gcttttttgataaccgcgaaaacatgttctctgaagaggccaaaagcacc |
| | | | tcaattgcgtttcgctgcattaacgaaaatctgacgcgttatatctctaa |
| | | | catggatatcttcgagaaggttgacgctatctttgataaacatgaagtcc |
| | | | aggagatcaaggagaagatcctgaacagcgactacgacgtggaggatttc |
| | | | ttcgagggtgagttcttcaattttgttttgacccaagaaggcatcgatgt |
| | | | ctacaatgcgataattggtggttttgttaccgagagcggggagaaaatta |
| | | | agggtctgaacgaatatatcaacttgtacaaccagaaaaccaaacaaaaa |
| | | | ctgccgaagtttaagccgctgtacaagcaagtcttgtcggatcgtgaatc |
| | | | gctgagcttttacggcgaaggctacaccagcgatgaggaggtgttggagg |
| | | | tgttccgcaatacgttgaataagaactctgaaattttttccagcattaag |
| | | | aagctggaaaaactattcaagaacttcgacgagtatagcagcgcaggtat |
| | | | ctttgtgaaaaacggtccggctattagcaccatctcgaaagacattttcg |
| | | | gcgaatggaatgttattcgtgataagtggaatgccgaatacgacgacatt |
| | | | catctgaagaaaaaggcggttgtaactgaaaagtacgaggacgatcgtcg |
| | | | taagtcgttcaaaaagatcggctccttctctctggagcaactgcaagagt |
| | | | acgccgatgccgatttgtcagtcgtggagaagctgaaagagatcatcatt |
| | | | caaaaagttgacgaaatctataaggtctatggtagcagtgaaaaactgtt |
| | | | tgacgcggacttcgtgctggagaaaagcctgaagaaaaatgacgccgtgg |
| | | | tggcaatcatgaaagatcttctggatagcgtcaagtctttcgaaaactac |
| | | | atcaaagcgtttttcggtgaaggcaaggaaacgaatcgtgacgagagctt |
| | | | ctatggtgatttcgtcctagcctatgatatcctgcttaaggtggatcaca |
| | | | tctatgatgccatccgcaactatgttacccagaaaccgtactcaaaggac |
| | | | aagttcaagttatacttccagaatccgcagtttatgggtggctgggataa |
| | | | agataaggaaaccgactatcgcgccaccattttacgttacggtagtaagt |
| | | | actatctggcaatcatggataaaaaatatgcgaaatgcctgcaaaagatc |
| | | | gacaaggacgatgttaacggcaattatgagaagatcaactacaaactgct |
| | | | gccgggcccaaataagatgctgccgaaggtgtttttcagcaaaaaatgga |
| | | | tggcgtattacaatccgagcgaggacatccaaaagatctacaaaaacggt |
| | | | acgtttaagaagggtgatatgttcaacctcaacgattgtcataaacttat |
| | | | tgacttcttcaaggatagcatatctcgttatccgaagtggagcaacgcgt |
| | | | atgactttaactttagcgaaaccgagaaatacaaagatatcgcgggtttc |
| | | | tatcgtgaagttgaagaacagggttacaaagtgtccttcgagtccgcgag |
| | | | caagaaggaggtagacaagctggtcgaggagggtaagttgtacatgtttc |
| | | | agatttacaataaggacttttccgacaagtcccatggtacaccgaatttg |
| | | | cacaccatgtatttcaagcttctgttcgacgagaacaaccacggccagat |
| | | | ccgtctgagcggtggtgcagagctgttcatgcgtagagctagcctgaaga |
| | | | aagaagagttggtcgttcatccggctaattcgccgatcgcgaacaaaaat |
| | | | ccagataaccctaaaaagactacgaccctgagctatgatgtgtataaaga |
| | | | caaacgctttagcgaagaccagtatgagttacacattccgatcgctatca |
| | | | acaagtgcccgaagaatattttcaagatcaacaccgaagtgcgtgttctt |
| | | | ttgaagcacgatgacaacccgtatgtgattggtattgctcgtggtgaacg |
| | | | taatctgctgtacattgtggttgtggacggcaaaggaaacatcgtggaac |
| | | | agtacagcctgaacgaaatcattaacaatttcaacggcattcgcattaag |
| | | | acggattaccattctttgctggacaaaaaggagaaagagcgtttcgaggc |
| | | | ccgtcagaactggacctccatcgagaacattaaggagctgaaggcgggtt |
| | | | acatcagccaggtggtgcacaaaatctgcgaactggtggagaagtacgac |
| | | | gcggtgattgctttggcggatttgaactccggttttaagaacagccgcgt |
| | | | taaagtggaaaagcaggtttatcagaaattcgaaaaaatgctgattgata |
| | | | aattgaattacatggttgacaaaaagtctaacccgtgtgcaaccggcggt |
| | | | gcgctgaagggctaccaaatcaccaataagtttgaaagctttaagagcat |
| | | | gagcacgcaaaatggcttcattttttacattccggcgtggctgaccagca |
| | | | agatcgatccgtcaaccggctttgttaatttactgaagacgaagtatacc |
| | | | tctattgccgactctaaaaaatttattagttccttcgatagaatcatgta |
| | | | tgttccggaagaggacctgttcgaatttgcactggactacaagaacttta |
| | | | gccgtaccgatgctgattatataaagaaatggaaactctacagctatggc |
| | | | aaccgcatccgtatctttcgcaacccgaaaaaaaacaacgtgttcgactg |
| | | | ggaagaggtatgtctgacttcagcgtacaaggagcttttcaataaatatg |
| | | | gtatcaactaccagcagggtgatatccgtgcactgctttgcgagcaaagc |
| | | | gataaagcgttctattcaagcttcatggcgctgatgagcctgatgctgca |
| | | | aatgcgcaacagcatcaccggtcgtaccgatgttgcatttctgatttctc |
| | | | cagtaaaaaacagcgatggtatcttttacgacagccgtaattacgaggcc |
| | | | caagaaaatgcgattttgccgaagaacgctgacgcgaatggtgcatacaa |
| | | | cattgcaaggaaagttctgtgggcaatcggccaatttaaaaaagcggaag |
| | | | atgagaagctggacaaagtgaaaattgcgattagcaacaaggagtggctg |
| | | | gagtatgcgcagacctccgttaaatcagggggaagtggcggaagcggtgg |
| | | | gtctacgaacctgagcgacattattgaaaaagaaactggtaaacagttgg |
| | | | tgatccaagaatccattctgatgttaccggaggaagtggaagaggtgatc |
| | | | ggcaacaagccggagtccgacatcctggttcacaccgcgtatgatgagtc |
| | | | gaccgatgagaatgttatgctgctgaccagcgacgccccagagtacaaac |
| | | | cgtgggcgctcgtcatccaggatagcaatggcgaaaacaagatcaagatg |
| | | | ttgtctggcggtagcatggctccgaaaaagaagcgtaaagtaggtattca |
| | | | tggtgttccggcatcaggttaa |
| 61 | Purification | HisX6 | atgggaagtcatcaccaccaccaccattcaggt |
| | tag | | |
| 62 | Nuclear | Nucleoplasmin | tctggcggtagcatggctccgaaaaagaagcgtaaagtag |
| | localization | -NLS | gtattcatggtgttccggca |
| | sequence | | |
| 63 | Linker | SGGSx2 | tcagggggaagtggcggaagcggtgggtct |
| | sequence | | |
| 64 | Nuclear | SV40-NLS | atggctccgaaaaagaaacgtaaggtgggtatccacggcgttccggcg |
| | localization | | |
| | sequence | | |
| 65 | Protease | TEV-RS | gagaacctgtacttccagagc |
| | cleavage | | |
| | site | | |
| 66 | Accessory | UGI | acgaacctgagcgacattattgaaaaagaaactggtaaacagttggtg |
| | component | | atccaagaatccattctgatgttaccggaggaagtggaagaggtgatc |
| | | | ggcaacaagccggagtccgacatcctggttcacaccgcgtatgatgag |
| | | | tcgaccgatgagaatgttatgctgctgaccagcgacgccccagagtac |
| | | | aaaccgtgggcgctcgtcatccaggatagcaatggcgaaaacaagatc |
| | | | aagatgttg |
| 67 | Linker | XTEN-linker | tctggaagtgagacacccgggacttcagaatccgcgaccccggag |
| | sequence | | |
| 68 | Deaminase | A3A | atgggaagtcatcaccaccaccaccattcaggtgagaacctgtacttcca |
| | | | gagcatggctccgaaaaagaaacgtaaggtgggtatccacggcgttccgg |
| | | | cgatggaagcaagtccagcttcaggacccaggcacctgatggacccgcat |
| | | | attttcacctctaattttaacaacggtattggccgtcacaagacgtacct |
| | | | gtgttatgaagtggaacgtcttgacaacggcacctccgtgaaaatggacc |
| | | | agcatcgtggtttcttgcataatcaagctaaaaacctgctgtgcggtttc |
| | | | tacggccgtcacgcggaactgcgttttttggacctggttccgagcctgca |
| | | | actggacccggcacagatttatcgtgttacttggttcatcagctggtcgc |
| | | | cgtgttttagctggggttgcgccggcgaggtacgcgcgtttttgcaagag |
| | | | aacacccacgtgcgtttgcgcatcttcgccgctcgtatctacgattatga |
| | | | cccactgtataaagaagcgctccagatgctgcgcgatgcaggcgcgcagg |
| | | | tttccatcatgacctacgacgagttcaagcactgctgggatacctttgtc |
| | | | gatcatcagggctgcccgttccagccgtgggatggtctggatgagcacag |
| | | | ccaggcgttaagcggtcgcctcagagcgattctgcaaaaccaaggtaatt |
| | | | ctggaagtgagacacccgggacttcagaatccgcgaccccggagtctgga |
| 69 | Deaminase | A3A-Y130F | atgggaagtcatcaccaccaccaccattcaggtgagaacctgtacttcca |
| | | | gagcatggctccgaaaaagaaacgtaaggtgggtatccacggcgttccgg |
| | | | cgatggaagcatcaccagctagtggacccaggcacctgatggatccgcac |
| | | | atcttcacctccaattttaataacggcatcggccgtcataagacgtacct |
| | | | gtgctatgaggttgaacgtttggacaacggtactagcgtcaagatggacc |
| | | | agcatcgtggtttccttcacaaccaagcgaaaaatctgctgtgcggtttt |
| | | | tatggtcgtcacgccgaactgagattcttggatctggttccgtctctgca |
| | | | actggacccggcgcagatttatcgtgtgacctggttcatcagctggtcgc |
| | | | cgtgctttagctggggttgcgccggtgaagtgcgcgcatttctccaggag |
| | | | aacacccatgttcgtttgcgcatcttcgcggcacgtatcttcgattacga |
| | | | cccgctgtacaaggaggcgctgcaaatgttgcgtgatgctggcgctcaag |
| | | | ttagcattatgacctacgacgaatttaaacactgttgggataccttcgtg |
| | | | gatcatcagggctgtccgtttcagccatgggacggcctggacgagcacag |
| | | | ccaggcgttatccggtcgcctgcgcgcgattctgcaaaaccagggaaact |
| | | | ctggaagtgagacacccgggacttcagaatccgcgaccccggagtctgga |
| 70 | Deaminase | A3B | atgggaagtcatcaccaccaccaccattcaggtgagaacctgtacttcca |
| | | | gagcatggctccgaaaaagaaacgtaaggtgggtatccacggcgttccgg |
| | | | cgatggaaatactaaggtatttaatggatcccgacactttcaccttcaat |
| | | | ttcaacaacgatccattggtcttgcgccgtcgtcagacctacttgtgcta |
| | | | tgaggttgaacgtctggataacggtacgtgggttttaatggatcagcata |
| | | | tgggtttcctgtgtaatgaagcgaagaacctgctgtgcggtttttacggc |
| | | | cgtcatgcggaactgcgttttcttgacctggttccgagcctgcaactgga |
| | | | cccggctcagatttaccgtgttacctggtttatctcgtggagcccgtgtt |
| | | | ttagctggggctgcgcgggtgaagtgcgcgcgtttttgcaagagaacacc |
| | | | cacgtgcgtctccgcatcttcgccgcacgtatttacgattatgacccgct |
| | | | gtataaagaggccctgcaaatgctgagagatgctggcgcacaagtgagca |
| | | | ttatgacctacgacgagttcgagtactgctgggatacgttcgtgtatcgc |
| | | | caaggttgcccgtttcagccgtgggacggcctggaggagcacagccaggc |
| | | | gctctccggtcgtttgcgcgcgatcctgcaatctggaagtgagacacccg |
| | | | ggacttcagaatccgcgaccccggagtctgga |
| 71 | Deaminase | AID | atgggaagtcatcaccaccaccaccattcaggtgagaacctgtacttcca |
| | | | gagcatggctccgaaaaagaaacgtaaggtgggtatccacggcgttccgg |
| | | | cgatggattcactattaatgaataggagaaaattcctgtatcagtttaag |
| | | | aacgtccgttgggctaaaggtcgtcgtgagacgtatttatgctacgtggt |
| | | | gaaacgtcgcgacagcgcaacctccttcagcctggacttcggctatctgc |
| | | | gtaacaaaaacggctgccacgtggagctgctgtttttgcgctacattagc |
| | | | gactgggatttggacccaggtcgctgctatcgtgttacctggtttacgag |
| | | | ctggtcgccgtgttacgactgtgcgcgtcatgtcgcggattttctgcgcg |
| | | | gtaatccgaatctgtccctgcgtatttttaccgcacgtctctacttctgc |
| | | | gaagatcgtaaggcggaaccggaaggcttgcgtcgcctgcatagagcggg |
| | | | tgttcagatcgctatcatgaccttcaaagattatttctactgctggaata |
| | | | ccttcgtggagaaccacgagcgtactttcaaggcctgggaaggccttcac |
| | | | gaaaacagcgttcgcttgtctcgtcaactgcgccgcatcctgctgccgct |
| | | | gtacgaggttgatgatctcagagacgcgtttcgtaccctgggtttgtctg |
| | | | gaagtgagacacccgggacttcagaatccgcgaccccggagtctgga |
| 72 | Deaminase | AmA1 | atgggaagtcatcaccaccaccaccattcaggtgagaacctgtacttcca |
| | | | gagcatggctccgaaaaagaaacgtaaggtgggtatccacggcgttccgg |
| | | | cgatggctgattcaagtgaaaaaatgaggggacaatatatcagccgtgac |
| | | | acctttgagaaaaactacaaaccgattgacggcacgaaagaggcgcatct |
| | | | gctgtgcgaaattaaatggggtaagtacggcaagccgtggctgcattggt |
| | | | gtcaaaaccaacgtatgaatatccatgctgaagactacttcatgaacaac |
| | | | atcttcaaagccaaaaagcatccggtgcactgctacgtgacctggtattt |
| | | | gtcctggtcaccgtgcgcggattgtgcgagcaagatcgttaagttcctgg |
| | | | aggagcgtccttatctgaaactgaccatttatgttgcacagctgtattat |
| | | | cacactgaagaggagaaccgtaaaggtctgcgtttgctgcgttcgaagaa |
| | | | ggtgattatccgcgtgatggatatctctgattacaactactgctggaaag |
| | | | tctttgttagcaatcagaacggtaatgaagactactggccgctgcaattc |
| | | | gatccgtgggttaaggagaactacagccgcctcttggacattttctggga |
| | | | aagcaagtgccgttccccaaatccgtggtctggaagtgagacacccggga |
| | | | cttcagaatccgcgaccccggagtctgga |
| 73 | Deaminase | Anc-rA1 | atgggaagtcatcaccaccaccaccattcaggtgagaacctgtacttcca |
| | | | gagcatggctccgaaaaagaaacgtaaggtgggtatccacggcgttccgg |
| | | | cgatgagttcagaaacaggacccgttgctgtagacccgacgctgcgccgt |
| | | | cgtattgagccgcacgagttcgaggttttttttgatccgcgtgaactccg |
| | | | caaggagacatgcttgctgtacgagatcaaatggggcacttcacacaaaa |
| | | | tctggcgccacagcagcaagaacaccaccaagcacgtggaagtgaacttc |
| | | | atcgagaaattcacgtctgaacgtcacttttgtccgtccaccagctgtag |
| | | | cattacctggtttctatcctggagcccgtgcggtgaatgttccaaggcga |
| | | | ttaccgaattcttgtcgcaacatccgaacgtgactcttgttatttatgtg |
| | | | gcccgtttgtaccatcacatggatcagcagaatcgtcaaggcttgcgcga |
| | | | cctggttaacagcggtgttaccatccagatcatgaccgcaccggaatacg |
| | | | actactgctggcgtaatttcgtcaactatccgccgggcaaggaggcgcat |
| | | | tggccgcgttatccgccgctgtggatgaaactgtacgctctggaactgca |
| | | | tgcgggtattttaggcctgccaccgtgcttgaatattctgagacgcaagc |
| | | | agcctcagctgacctttttcacgatcgcgctgcaaagctgtcattatcaa |
| | | | cgtctgccgccacacatcctgtgggctaccggtctgaaatctggaagtga |
| | | | gacacccgggacttcagaatccgcgaccccggagtctgga |
| 74 | Deaminase | eA3A | atgggaagtcatcaccaccaccaccattcaggtgagaacctgtacttcca |
| | | | gagcatggctccgaaaaagaaacgtaaggtgggtatccacggcgttccgg |
| | | | cgatggaagcaagtcccgcttcaggaccaaggcacctgatggacccacat |
| | | | attttcaccagtaattttaacaacggcatcggccgtcacaagacctacct |
| | | | gtgttatgaggtcgaacgtctggataatggcacttccgtgaaaatggatc |
| | | | agcatcgtggtttcctgcatggtcaggctaagaatctgctttgcggtttc |
| | | | tatggtcgccacgccgaactgcgttttttggacctggttccgtccctcca |
| | | | actggacccggcgcaaatctatcgtgttacctggtttatcagctggtcgc |
| | | | cgtgtttcagctggggttgcgcaggcgaagtacgtgcgttcttacaagag |
| | | | aacacccatgtgcgcttgcgcatcttcgccgcacgtatttacgactacga |
| | | | cccgctgtataaagaggcgttgcaaatgctgcgcgatgctggcgcacagg |
| | | | ttagcattatgacgtacgacgaatttaagcactgctgggataccttcgtg |
| | | | gatcaccagggctgcccgtttcagccgtgggatggtttggacgagcactc |
| | | | tcaggcgctgagcggtcgtctcagagcgattctgcaaaaccagggaaact |
| | | | ctggaagtgagacacccgggacttcagaatccgcgaccccggagtctgga |
| 75 | Deaminase | evo APOBEC 1 | atgggaagtcatcaccaccaccaccattcaggtgagaacctgtacttcca |
| | | | gagcatggctccgaaaaagaaacgtaaggtgggtatccacggcgttccgg |
| | | | cgatgtcaagtaaaacaggacccgttgctgtagatccgacgctgagacgt |
| | | | cgcatcgagccgcacgaattcgaggtgttttttgatccgcgtgaactgcg |
| | | | caaggagacgtgcctgctgtatgagatcaactggggtggccgtcacagca |
| | | | tttggcgtcacacctcccaaaacacgaacaaacacgtggaagtcaatttc |
| | | | atcgaaaaattcaccaccgaacgttacttctgcccgaacacccgttgttc |
| | | | catcacctggttcctgtcttggagcccgtgtggtgagtgcagccgcgcaa |
| | | | ttactgagttcctgagccgttacccgaatgttaccctgtttatctatatc |
| | | | gcccgtttgtaccatctggcgaacccgcgcaaccgtcagggtttacgcga |
| | | | cttaattagctctggcgttaccattcagattatgaccgagcaagagagcg |
| | | | gttattgctggcataactttgtaaactatagcccgagcaatgaaagccat |
| | | | tggcctcgctacccgcacctgtgggttcgtttgtacgtgctggaactgta |
| | | | ctgcatcatccttggtttgccaccgtgcttgaatattttgagacgtaagc |
| | | | agtcgcaactgaccagttttacgatcgcgctacagagctgtcattatcag |
| | | | cgtctcccgccacacattctgtgggctaccggcctgaaaagcggcggtag |
| | | | cagcggtggctccagcggctcggaaactccgggcacctccgagtcagcga |
| | | | ccccggaatctggaagtgagacacccgggacttcagaatccgcgaccccg |
| | | | gagtctgga |
| 76 | Deaminase | evoCDA1 | atgggaagtcatcaccaccaccaccattcaggtgagaacctgtacttcca |
| | | | gagcatggctccgaaaaagaaacgtaaggtgggtatccacggcgttccgg |
| | | | cgatgtcaacagatgctgaatatgtaaggatacacgagaagctggacatc |
| | | | tacacctttaaaaagcagttctccaacaataaaaaatctgtcagccatcg |
| | | | ttgctacgtgttgtttgaactcaagcgtcgtggcgaacgccgtgcgtgct |
| | | | tttggggttacgctgtgaacaaaccgcagagcggtacggaacgtggtatt |
| | | | catgcggagatcttcagcatccgcaaggtggaagagtatctgcgcgataa |
| | | | cccgggtcaattcaccattaactggtacagcagctggtcaccgtgcgccg |
| | | | actgtgctgagaagattttggagtggtataaccaagaattgcgcggtaat |
| | | | ggccatactctgaagatctgggtgtgtaaactttattacgaaaagaacgc |
| | | | gcgtaatcaaattggtctgtggaatctgagagataacggcgttggtctga |
| | | | atgttatggtttccgagcactatcagtgctgccgtaaaatcttcattcag |
| | | | tccagccacaaccaactgaacgagaaccgttggctggaaaagaccctgaa |
| | | | gcgcgcggagaaacgtcgtagcgagctgagcattatgtttcaggttaaaa |
| | | | tcttacacaccaccaaatcgccggcagtctctggcggttcctcgggcggc |
| | | | agctctggctccgaaaccccaggtacgagcgagagcgcaaccccggaatc |
| | | | tggaagtgagacacccgggacttcagaatccgcgaccccggagtctgga |
| 77 | Deaminase | evoFERNY | atgggaagtcatcaccaccaccaccattcaggtgagaacctgtacttcca |
| | | | gagcatggctccgaaaaagaaacgtaaggtgggtatccacggcgttccgg |
| | | | cgatgtcatttgagaggaattatgatcccagagaattgcgcaaagagacg |
| | | | tacctgctgtatgaaatcaagtggggtaaaagcggcaagctgtggcgtca |
| | | | ttggtgtcaaaacaaccgcacccaacacgcggaagtttatttcctcgaga |
| | | | atattttcaacgcccgtcgttttaacccgagcacccactgctccatcacc |
| | | | tggtatctttcttggtcgccgtgtgcggagtgctcgcaaaaaatcgtgga |
| | | | cttcctgaaagaacatccgaatgtgaacctggagatctatgttgcgagat |
| | | | tgtactatccggagaacgaacgtaatcgtcagggcctgcgcgacttggtc |
| | | | aacagcggggtgaccattcgtattatggatctgccggattacaattactg |
| | | | ctggaaaacctttgtttccgatcagggtggcgacgaagactactggcctg |
| | | | gtcactttgctccgtggattaagcagtacagcctgaagctgagcggtggt |
| | | | tcttccggcggtagcagtggctccgagactccaggtacgagcgagagcgc |
| | | | aaccccggaatctggaagtgagacacccgggacttcagaatccgcgaccc |
| | | | cggagtctgga |
| 78 | Deaminase | hA3G-CTD | atgggaagtcatcaccaccaccaccattcaggtgagaacctgtacttcca |
| | | | gagcatggctccgaaaaagaaacgtaaggtgggtatccacggcgttccgg |
| | | | cgatggatcccccaacatttactttcaactttaataacgaaccgtgggtt |
| | | | cgcggtcgccacgaaacctacctgtgctatgaggtggaacgtatgcataa |
| | | | tgatacttgggttctgctgaatcaacgtagaggctttttatgcaaccaag |
| | | | ctccgcataaacatggttttctggaaggccgtcacgccgagctgtgcttc |
| | | | cttgacgtgatcccattctggaaactggacttggaccaggattatcgtgt |
| | | | gacctgtttcaccagctggtcgccgtgcttcagctgtgcgcaggagatgg |
| | | | cgaagttcatctccaaaaacaagcacgttagcttgtgcatttttaccgcg |
| | | | cgtatttacgatgatcaaggtcgctgccaagagggcctgcgtaccctggc |
| | | | agaggctggtgcgaagatctctatcatgacgtacagcgaatttaaacact |
| | | | gctgggacacgtttgtcgatcaccagggttgtccgttccagccgtgggac |
| | | | ggcttggacgaacatagccaggatttgtccggtcgtctgcgcgcaattct |
| | | | acagaatcaagagaactctggaagtgagacacccgggacttcagaatccg |
| | | | cgaccccggagtctgga |
| 79 | Deaminase | minStart | atgggcagccaccaccaccaccaccacagcggcgagaacctgtacttcca |
| | | | gtccatggctccaaagaagaagagaaaggttggaatccacggagttccag |
| | | | cttctgga |
| 80 | Deaminase | mTadA8e | atgggaagtcatcaccaccaccaccattcaggtgagaacctgtacttcca |
| | | | gagcatggctccgaaaaagaaacgtaaggtgggtatccacggcgttccgg |
| | | | cgagcggattagtaatgcaaaattataggctaatagacgctacgttgtat |
| | | | gtgacctttgaaccgtgcgttatgtgcgctggtgcaatgattcattctcg |
| | | | tatcggccgtgtcgtgttcggcgtgagaaacagcaagcgcggtgcggcgg |
| | | | gtagcctgatgaatgttctgaactacccgggtatgaaccaccgtgttgaa |
| | | | attaccgagggtatcttggcagacgagtgcgccgccctgctgtgtgattt |
| | | | ctaccgcatgccgcgtcaggtttttaacgcgcagaaaaaggcgcaaagca |
| | | | gcatcaatagcggcggtagctctggcggctcctccggttcggagtctgga |
| | | | agtgagacacccgggacttcagaatccgcgaccccggagtctgga |
| 81 | Deaminase | pmCDA1 | atgggaagtcatcaccaccaccaccattcaggtgagaacctgtacttcca |
| | | | gagcatggctccgaaaaagaaacgtaaggtgggtatccacggcgttccgg |
| | | | cgatgacagatgctgaatatgtaaggatacacgagaagttggacatctac |
| | | | acctttaaaaagcagttctttaacaacaaaaagagcgtcagccatcgttg |
| | | | ctatgttctgtttgagttgaagcgccgtggcgaacgtcgtgcgtgctttt |
| | | | ggggttatgctgtgaacaaaccgcagtctggtactgaacgcggtattcat |
| | | | gcagaaattttcagcattcgtaaggtggaagaatacctgcgcgataaccc |
| | | | aggtcagttcaccattaactggtacagcagctggtccccgtgcgcggatt |
| | | | gcgctgagaagatcttggagtggtacaatcaagagctgcgtggtaatggc |
| | | | cataccctgaaaatctgggcgtgcaaactgtattacgaaaagaacgcgcg |
| | | | taatcaaattggtctgtggaatctgcgtgacaacggcgtgggcctgaatg |
| | | | ttatggtttccgagcactatcagtgttgtcgtaaaatcttcatccaatcc |
| | | | tctcacaaccaattgaacgagaaccgctggctggagaagaccctgaagcg |
| | | | cgccgagaaaagacgtagcgaactctcgatcatgattcaggttaaaatct |
| | | | tacacacgaccaaaagcccggcagtgtctggaagtgagacacccgggact |
| | | | tcagaatccgcgaccccggagtctgga |
| 82 | Deaminase | PpA1-R33A | atgggaagtcatcaccaccaccaccattcaggtgagaacctgtacttcca |
| | | | gagcatggctccgaaaaagaaacgtaaggtgggtatccacggcgttccgg |
| | | | cgatgacaagtgaaaaaggaccctcaactggggacccaaccctgcgtcgt |
| | | | cgcattgaaagctgggagttcgacgttttttatgatccgcgtgagctggc |
| | | | gaaagagacgtgcctgttgtacgagatcaaatggggtatgagccgtaaaa |
| | | | tctggagatccagcggtaaaaacaccaccaatcacgtggaagtgaatttt |
| | | | atcaagaagttcaccagcgaacgtcgtttccacagctccatttcttgttc |
| | | | tattacctggtttctgtcttggtcgccgtgttgggagtgctcccaggcaa |
| | | | ttcgtgaattccttagtcaacatccgggcgtgaccctggtcatctacgtc |
| | | | gcccgtttgttttggcatatggaccagcgcaatcgtcagggtttgcgcga |
| | | | tctggttaacagcggcgttacgatccaaattatgcgtgcgagcgagtatt |
| | | | accactgctggcgcaacttcgtgaactatccaccgggcgatgaagcacat |
| | | | tggccgcaatatccgccgctgtggatgatgctgtacgcgctggaactcca |
| | | | ctgcattatcctgtcgctgccgccttgtctgaagatcagccgacgctggc |
| | | | agaaccacctcgcgtttttccgcctgcacttgcaaaactgccattaccag |
| | | | accatcccgccgcatatcctgttagctaccggtctgattcacccgagcgt |
| | | | tacttggcgttctggaagtgagacacccgggacttcagaatccgcgaccc |
| | | | cggagtctgga |
| 83 | Deaminase | rA1 | atgggaagtcatcaccaccaccaccattcaggtgagaacctgtacttcca |
| | | | gagcatggctccgaaaaagaaacgtaaggtgggtatccacggcgttccgg |
| | | | cgatgagttcagaaacaggacccgttgctgtagacccgaccctgcgtcgc |
| | | | cgcattgagccgcacgagttcgaagtcttttttgatccgcgtgaactgcg |
| | | | caaagaaacctgtctgctgtatgaaattaactggggtggccgtcacagca |
| | | | tttggcgtcacactagtcaaaacaccaataaacacgtggaagttaacttc |
| | | | atcgagaaatttaccaccgagcgctacttctgcccgaatactcgttgttc |
| | | | tattacgtggtttctctcttggtcgccgtgcggcgagtgcagccgtgcga |
| | | | ttaccgagttcctgagccgttacccgcacgttaccctgtttatctatatc |
| | | | gcgcgtctgtaccatcatgcggacccgcgtaaccgtcaaggtctgcgtga |
| | | | tctgatcagcagcggtgttacgatccagattatgacggagcaggagtccg |
| | | | gctattgctggcgcaacttcgtgaactatagcccatccaatgaagctcat |
| | | | tggccgcgctacccgcacctgtgggtacgtttgtacgtgctggaactcta |
| | | | ctgcatcatccttggcctgccaccgtgcttgaatattttgagacgcaagc |
| | | | agccgcaactgaccttcttcaccatcgccctgcaaagctgtcattatcag |
| | | | cgtttgccgcctcacatcttatgggcaaccggtttgaagtctggaagtga |
| | | | gacacccgggacttcagaatccgcgaccccggagtctgga |
| 84 | Deaminase | rA1-YE1 | atgggaagtcatcaccaccaccaccattcaggtgagaacctgtacttcca |
| | | | gagcatggctccgaaaaagaaacgtaaggtgggtatccacggcgttccgg |
| | | | cgatgagttcagaaacaggacccgttgctgtagacccgaccttgcgcaga |
| | | | cgtattgagccgcacgaattcgaggtgtttttcgacccgagagaattgcg |
| | | | caaagaaacctgtctgctgtacgagatcaactggggtggtcgtcatagca |
| | | | tctggcgtcacacctctcagaatacgaacaaacacgtggaagtcaatttc |
| | | | atcgagaagttcacgaccgagcgctacttctgtccgaacacccgttgttc |
| | | | gatcacgtggtttctgagctattctccgtgcggcgaatgcagccgtgcaa |
| | | | ttaccgagttcctgtcacgttacccgcacgtaactctgtttatctacatt |
| | | | gcgcgtctctaccatcatgctgatccggaaaaccgtcaaggtttacgcga |
| | | | tctgattagctccggcgtgaccatccagattatgactgagcaggagagcg |
| | | | gctattgctggcgcaactttgttaactattccccgtccaatgaggcgcat |
| | | | tggcctcgttatccgcacctgtgggttcgtctatacgttctggaattgta |
| | | | ctgcattatcctgggcctgccaccatgcttgaatattctgcgtcgcaagc |
| | | | agccgcaattgacctttttcaccatcgccctgcaaagctgccattatcag |
| | | | cgccttccgccgcacatcctgtgggcgaccggtctgaaatctggaagtga |
| | | | gacacccgggacttcagaatccgcgaccccggagtctgga |
| 85 | Deaminase | rA1-YEE | atgggaagtcatcaccaccaccaccattcaggtgagaacctgtacttcca |
| | | | gagcatggctccgaaaaagaaacgtaaggtgggtatccacggcgttccgg |
| | | | cgatgtcaagtgaaacaggacccgttgctgtagacccgaccttgagacgc |
| | | | cgcattgagccgcacgaatttgaagttttcttcgacccgcgtgaattgcg |
| | | | taaagagacgtgcttgctgtacgagattaactggggtggccgtcacagca |
| | | | tttggcgccacaccagccaaaacaccaacaaacatgtggaagtcaacttc |
| | | | atcgagaagttcactaccgagcgttacttttgtccgaatacgcgttgttc |
| | | | gatcacctggtttctgtcctattccccgtgcggtgaatgctcacgcgcaa |
| | | | tcactgagttcctgtctcgttacccgcacgtaaccctgtttatctatatc |
| | | | gcgcgtttgtaccatcatgcggatccggagaaccgccagggtctggaaga |
| | | | tttaattagcagcggcgttacgatccagattatgaccgagcaggagagcg |
| | | | gctattgctggcgtaatttcgtgaactattccccgtctaatgaagcgcat |
| | | | tggccgcgttacccacacctgtgggttcgtctctacgtgctggaattgta |
| | | | ctgcattatcctgggtttaccgccttgcctgaatattctacgtcgcaagc |
| | | | aaccgcagctgacctttttcaccatcgccctgcaaagctgtcattatcag |
| | | | cgtctgccgccacacatcctgtgggctaccggccttaaatctggaagtga |
| | | | gacacccgggacttcagaatccgcgaccccggagtctgga |
| 86 | Deaminase | RrA3F-F130L | atgggaagtcatcaccaccaccaccattcaggtgagaacctgtacttcca |
| | | | gagcatggctccgaaaaagaaacgtaaggtgggtatccacggcgttccgg |
| | | | cgatgaaacctcagattcgcgatcatcgtccgaatcctatggaggcgatg |
| | | | tatccgcatattttctactttcattttgaaaacttagaaaaagcctatgg |
| | | | ccgtaacgaaacttggctgtgctttaccgtagagattatcaaacaatatt |
| | | | taccggtaccctggaaaaaaggtgttttccgtaatcaggttgatccggag |
| | | | acacattgtcatgcagagaagtgcttcctgtcctggttctgcaataacac |
| | | | ccttagcccgaaaaagaattaccaagttacttggtacacgagctggtcgc |
| | | | catgtccagagtgcgcaggagaagtagcagaattcttagctgaacattcg |
| | | | aacgtaaaattgacaatctacaccgcccgtctctactacctgtgggacac |
| | | | cgactatcaggaaggcctgcgctcattgtctgaggagggcgcatcggtcg |
| | | | aaatcatggattatgaagattttcagtattgttgggaaaatttcgtatac |
| | | | gacgatggtgagccttttaaacgttggaaaggcctgaagtacaactttca |
| | | | gtctttgacccgtcgcttgcgcgaaattctgcaatctggaagtgagacac |
| | | | ccgggacttcagaatccgcgaccccggagtctgga |
| 87 | Deaminase | SsA3B-R54Q | atgggaagtcatcaccaccaccaccattcaggtgagaacctgtacttcca |
| | | | gagcatggctccgaaaaagaaacgtaaggtgggtatccacggcgttccgg |
| | | | cgatggatccacagaggctaagacaatggcccggtccgggtccggcgagc |
| | | | cgcggtggctacggccagcgtccgcgtatccgcaatccggaagagtggtt |
| | | | ccacgaactgagtccgagaacctttagcttccactttcgtaacctgcgtt |
| | | | ttgctagcggtcagaatcgcagctatatctgctgccaggtggaaggcaag |
| | | | aactgcttcttccaaggtatttttcaaaaccaggttccgccggacccacc |
| | | | ttgtcatgctgaactttgctttttgtcctggttccaaagctggggtttgt |
| | | | cgccggacgagcactattacgtgacctggttcatctcctggtccccgtgc |
| | | | tgtgaatgtgcggcgaaagtcgcccaatttctggaagagaaccgcaacgt |
| | | | gagcttgtctttgtctgcggcgcgtctgtactacttctggaaaagcgagt |
| | | | cccgtgagggcctgcgccgtctgtccgatctgggtgcacaggttggtatt |
| | | | atgtcgttccaagattttcagcattgttggaataactttgttcataatct |
| | | | gggcatgccgttccagccgtggaagaaattgcacaaaaactaccaacgtc |
| | | | tggtgaccgaattaaaacagattctccgcgaggagccggctacctatggc |
| | | | tctccacaggcgcaaggtaaggtgcgcatcggcagcaccgcggcgggtct |
| | | | gcgtcacagccatagccacacccgtagcgaggcgcatctgcgtcccaacc |
| | | | actcatctcgtcagcaccgtatcctgaatccgccgcgcgaggcccgtgcg |
| | | | cgtacgtgcgttctggtagacgcatcctggatttgctatcgttctggaag |
| | | | tgagacacccgggacttcagaatccgcgaccccggagtctgga |
| 88 | Nuclease | dCas9 | atggataaaaagtattcaataggactagctattggcaccaacagcgttgg |
| | | | ttgggcagttatcaccgatgaatataaggtcccgagcaagaagtttaagg |
| | | | tcctgggcaacaccgatcgccacagcataaaaaagaaccttattggtgcg |
| | | | ctgctgttcgattctggtgagactgccgaagcgacccgtctgaagagaac |
| | | | cgcgcgtcgtagatacacccggcgcaagaaccgtatctgctatctgcaag |
| | | | aaattttcagcaacgagatggctaaggtcgacgactccttctttcatcgc |
| | | | ctggaggaaagctttctggttgaggaagacaaaaagcacgagcgtcatcc |
| | | | gattttcggtaacattgttgatgaagttgcctaccacgaaaaatacccca |
| | | | caatttatcatctgcgtaaaaaactggtagactccaccgataaggcggat |
| | | | ttacgccttatctatttggcgctggcgcatatgattaagttccgcggtca |
| | | | ttttttgatcgagggcgacctgaatccggataacagcgacgttgacaaac |
| | | | tgtttatccagctggtgcagacctataaccagttgttcgaagagaacccg |
| | | | attaatgcgtctggcgtggacgcgaaggcgatcctgagcgctcgtctgtc |
| | | | caagtcacgccgcctggagaacttgatcgcgcaactgccgggtgagaaga |
| | | | aaaacggcttgttcggcaatctgattgctctgtccctaggtttgaccccg |
| | | | aatttcaaatctaattttgatctggcggaagatgcgaaattacagttgag |
| | | | caaggacacttacgatgacgatctggacaacctgctcgctcagatcggcg |
| | | | atcagtatgccgatcttttcctggccgcgaagaacctgagcgatgccatc |
| | | | ttgctatcggacatcttacgtgttaacaccgaaatcaccaaagctccgct |
| | | | aagcgcgtccatgattaaacgttacgatgaacaccaccaggatctgacgc |
| | | | tgctgaaagcgttggtgcgccagcaactgccggaaaaatacaaggaaatt |
| | | | ttcttcgaccaaagcaagaacggttatgcaggctatatcgacggtggtgc |
| | | | gtcgcaagaagaattttataaattcatcaaaccgattttggagaagatgg |
| | | | atggtaccgaggagctgctggtaaaattgaatcgggaagacctcttgcga |
| | | | aagcagcgtactttcgataatggtagcatcccgcaccagattcatctggg |
| | | | cgaacttcacgcaatcctacgtcgtcaagaggatttctacccgtttctga |
| | | | aggataaccgtgaaaagatcgagaagattctgacctttcgtattccgtat |
| | | | tatgtgggtccgctggcgagaggcaacagccgttttgcgtggatgacccg |
| | | | caagtcggaagagactatcacgccgtggaatttcgaggaggtcgtggaca |
| | | | agggtgcatcagcgcaatcttttattgaacgtatgaccaattttgataaa |
| | | | aaccttccaaatgaaaaagtgttgccgaagcacagcttgctgtacgagta |
| | | | tttcaccgtttacaatgaactgacgaaagtcaagtacgttaccgaaggta |
| | | | tgcgtaagccagcctttctgtctggggagcaaaaaaaagccattgtggat |
| | | | ctgctgttcaagaccaatcgcaaggttaccgtcaagcaactgaaagagga |
| | | | ttacttcaaaaaaatcgaatgttttgactccgtggaaatcagcggcgttg |
| | | | aagaccgctttaatgcatctctgggcacctaccacgatctactcaagatc |
| | | | atcaaggataaggacttcctggacaacgaagaaaatgaggatatcttgga |
| | | | agatattgtgctgactttaacgttgttcgaggatcgtgagatgattgaag |
| | | | aacgcctcaaaacgtacgcacatctgtttgacgataaggtgatgaaacag |
| | | | ctaaagcgccgtcgttacaccggttggggtcgtctgtctagaaagctgat |
| | | | taacggcattcgtgacaaacaatctggcaagaccatcctggacttcctca |
| | | | aatccgacggcttcgcaaatcgtaatttcatgcaactgatccatgatgat |
| | | | agcctgacctttaaagaagatattcagaaggctcaagtgtccggccaggg |
| | | | tgactcgctgcatgagcatatcgcgaatctggctggctcccctgcgataa |
| | | | aaaaaggtattctccaaacggtgaaagttgtggatgaactggttaaagtc |
| | | | atgggtcgtcataaaccggaaaacatcgtgattgagatggcacgtgagaa |
| | | | ccagaccacccagaaaggtcaaaaaaacagtcgtgagcgtatgaaacgta |
| | | | tcgaagaggggattaaggagttaggcagccagattctgaaggagcacccg |
| | | | gttgagaacacccagctgcaaaacgagaagctctacctgtattacctgca |
| | | | aaacggccgtgacatgtatgttgaccaggagctggacatcaatcgtctca |
| | | | gcgactacgacgtggatgctatcgtgccgcagagctttctaaaagatgat |
| | | | tccatcgacaataaagtgctcacccgtagcgacaagaaccgtggtaaaag |
| | | | cgacaacgtgccgagcgaggaagtagttaagaaaatgaaaaactactggc |
| | | | gtcagctattgaacgccaagcttatcacccagcgtaagtttgacaacttg |
| | | | acgaaggcagagcgtggcggcctgagcgagctagacaaggcgggttttat |
| | | | taaacgccagctggtggaaactcgccagattaccaaacacgttgcgcaaa |
| | | | tcttggactcacgcatgaatacgaagtacgacgaaaacgataaattgatc |
| | | | cgcgaggtgaaggtcataaccctgaaatctaaactggttagcgatttccg |
| | | | gaaggactttcaattttataaagtccgggagattaacaactatcaccatg |
| | | | cacacgacgcttacttaaatgcggttgttggcacggccctgatcaagaag |
| | | | tacccgaaactggaaagcgagtttgtttatggtgactacaaggtgtatga |
| | | | cgttcgcaagatgattgcaaaatccgaacaagagattggtaaggcgacgg |
| | | | ctaagtattttttctacagcaatatcatgaacttcttcaagactgaaatc |
| | | | accctggcgaacggtgaaattcgtaaacgtccgttgatcgaaaccaacgg |
| | | | tgaaaccggtgaaatcgtttgggataaaggtagggattttgcgaccgttc |
| | | | gtaaggtcctgagcatgccgcaggttaacatcgtgaaaaagaccgaagtg |
| | | | cagaccggcggtttctccaaggagagtatcttaccgaagcgcaacagtga |
| | | | caagctgatcgcgcgtaaaaaagactgggatccgaaaaaatacggcggct |
| | | | ttgactccccgaccgtcgcgtacagcgtactggtcgtggctaaagtggag |
| | | | aaaggcaaatcgaaaaagctgaaatccgttaaagagttgctgggtattac |
| | | | gattatggaacgttcgagcttcgagaaaaatccgatcgacttcctggagg |
| | | | ccaaaggctacaaggaagttaaaaaagacctgatcattaaactgccgaag |
| | | | tacagcctgttcgagctcgagaacggtcgcaaacgcatgctggcgagcgc |
| | | | gggtgagctccaaaaaggcaacgaattggctctgccgagcaagtacgtga |
| | | | acttcctgtatctggcctcccattatgaaaaactgaagggtagcccggaa |
| | | | gataacgaacaaaagcagctgttcgtggaacaacataaacactatctgga |
| | | | cgagattattgagcaaatcagcgagttcagcaaacgcgtgattctggcgg |
| | | | atgctaatctggacaaggttctttcagcctataataaacaccgtgataag |
| | | | ccgatccgtgaacaggcagaaaatattattcacctgttcaccctgacgaa |
| | | | cctgggtgcgccagcagcgtttaaatacttcgacacaacgatcgaccgga |
| | | | agcgttataccagcaccaaagaggttcttgacgccaccttgattcaccaa |
| | | | agcatcacgggtctgtatgaaacccgtattgacttgagccagttgggcgg |
| | | | agattcagggggaagtggcggaagcggtgggtctacgaacctgagcgaca |
| | | | ttattgaaaaagaaactggtaaacagttggtgatccaagaatccattctg |
| | | | atgttaccggaggaagtggaagaggtgatcggcaacaagccggagtccga |
| | | | catcctggttcacaccgcgtatgatgagtcgaccgatgagaatgttatgc |
| | | | tgctgaccagcgacgccccagagtacaaaccgtgggcgctcgtcatccag |
| | | | gatagcaatggcgaaaacaagatcaagatgttgtctggcggtagcatggc |
| | | | tccgaaaaagaagcgtaaagtaggtattcatggtgttccggcatcaggt |
| 89 | Nuclease | dCasPhi-2 | atgccaaaacccgctgtagagtcagaattttcaaaagttttgaaaaagca |
| | | | ctttccgggagaacgtttccgctcgagctatatgaaaagaggcggtaaga |
| | | | tcctggctgcccaaggcgaagaggcagttgttgcgtatctacagggcaag |
| | | | tccgaggaggagcctccgaacttccagccaccggcgaagtgccacgtggt |
| | | | gaccaaatcccgtgacttcgcggaatggccgatcatgaaggcgtcggaag |
| | | | cgattcaacgttatatctatgcactgtcgaccaccgaacgcgcggcctgt |
| | | | aaaccgggcaaaagtagtgagagccatgcagcgtggttcgccgctaccgg |
| | | | tgtcagcaaccacggctacagccacgttcagggtctgaacctgatttttg |
| | | | atcataccttgggccgttacgatggtgttctgaaaaaggtccaactgcgc |
| | | | aacgaaaaggcgagagcgcgtttagagagcattaacgcgtctagagcaga |
| | | | tgaaggtttaccggagattaaagccgaggaggaagaagtggctacgaacg |
| | | | aaaccggccacctgttgcaaccgccgggtatcaatccgagcttttacgtg |
| | | | tatcagaccattagcccgcaggcataccgtccgcgtgacgagatcgtgct |
| | | | gccgccggagtacgcaggttacgtacgtgacccgaatgctccgattccgc |
| | | | tgggtgtggtgcgtaaccgttgcgatatccaaaaagggtgcccgggctac |
| | | | atcccggaatggcagcgtgaagcaggtaccgcgatcagcccgaaaacagg |
| | | | caaggccgtaactgtgccgggcctgtctccgaagaaaaacaaacgtatgc |
| | | | gtaggtactggcgctctgagaaggagaaagctcaagatgcgctgttggtt |
| | | | accgtacgcattggtaccgattgggttgtcatcgacgtgcgcggtctgtt |
| | | | gcgcaacgcccgttggcgcaccatcgctcccaaggacatcagcctgaatg |
| | | | cgctcctcgatttgtttaccggtgatccggtgatcgatgtgcgtcgcaat |
| | | | attgttacctttacgtacaccctcgacgcctgtggtacctacgcccgtaa |
| | | | atggaccttgaaaggcaaacagacgaaagcgaccctggacaaactgacgg |
| | | | ccactcagacggttgcgttggttgcgattgcgcttggtcagaccaacccg |
| | | | atttccgctggcattagccgtgttacacaagaaaatggtgcactgcaatg |
| | | | cgaaccgctggaccggttcaccctgccggatgacctgttgaaggacatca |
| | | | gcgcatatcgcattgcttgggacagaaacgaagaggaactacgcgctcgc |
| | | | agcgtcgaggccctgccggaggcgcaacaagcggaggtccgtgcgctgga |
| | | | cggggtgtccaaagaaaccgcgcgtacccaactgtgcgcagatttcggcc |
| | | | tggacccgaaacgtctgccgtgggataaaatgagctccaatactaccttt |
| | | | atcagcgaagccttgctgtctaacagcgtgagccgtgatcaggttttttt |
| | | | cactccggctccgaagaagggtgctaagaaaaaagcgcctgttgaagtta |
| | | | tgcgtaaagaccgcacttgggcacgtgcgtacaagccgcgtctgagcgtc |
| | | | gaagctcagaaactgaagaacgaagccctgtgggcacttaagcgcacctc |
| | | | cccggagtatttgaagctgagccgtcgcaaagaggagctgtgtcgtcgta |
| | | | gcatcaactatgttatcgaaaagacccgtcgccgtacgcaatgtcagatt |
| | | | gtgatcccggtgatcgaggatctgaatgttcgcttctttcatggttctgg |
| | | | taagagactgcccggttgggataatttcttcaccgctaaaaaagagaacc |
| | | | gttggttcatccagggcctgcacaaagcgtttagcgatctgcgtacgcac |
| | | | cgttccttctatgtttttgaagttcgtccggagcgcacctctattacctg |
| | | | tccaaagtgcggtcattgcgaggtgggtaatcgtgacggtgaggcgtttc |
| | | | agtgcctgagctgcggcaagacgtgcaacgcagatcttgacgtcgcgacc |
| | | | cataatctgacccaagtggcgttgaccggtaagaccatgccgaagcgcga |
| | | | ggagccgcgtgacgctcagggtacagcgccagcgcgcaagactaagaagg |
| | | | cgagcaaatcaaaggcgccaccggcggagcgcgaagaccagaccccggcg |
| | | | caagagccaagccagaccagttctggaggtagcgggggtagcggtggtag |
| | | | cactaacttaagtgatattatcgagaaagaaaccgggaaacagctggtga |
| | | | tccaagaaagcattctgatgctgcccgaagaagttgaagaagtgattggt |
| | | | aacaagcctgagtcggacatcttagtgcacaccgcgtacgatgaaagcac |
| | | | ggatgagaatgtgatgctgctcacctctgatgcccccgaatacaagccgt |
| | | | gggctctggtgatccaggactctaacggagaaaacaaaattaagatgctc |
| | | | tctggcggctccatggcgcctaaaaaaaaacgcaaagtggggattcacgg |
| | | | ggttccggcgtcaggt |
| 90 | Nuclease | dLbCas12a | atgtcaaaactagaaaagtttacaaattgttactccctgtcgaaaaccct |
| | | | gagattcaaagcgatcccggtgggtaaaactcaagagaacattgataaca |
| | | | aacgtctgctggttgaggacgaaaagcgcgcggaggattacaaaggcgtt |
| | | | aaaaagctgttggaccgttattacctgtcctttattaatgatgttctgca |
| | | | cagcattaaattaaagaacctaaacaattacatctccctcttccgtaaaa |
| | | | agacccgcaccgaaaaggaaaataaagaactggagaaccttgagattaat |
| | | | ctgcgtaaagaaatcgctaaagcgtttaaaggcaacgagggttataaatc |
| | | | cttatttaaaaaagacatcatcgaaacgattctcccagaattcctggacg |
| | | | acaaagacgagatcgcgctggttaattccttcaacggctttaccaccgca |
| | | | ttcaccggcttttttgataaccgcgaaaacatgttctctgaagaggccaa |
| | | | aagcacctcaattgcgtttcgctgcattaacgaaaatctgacgcgttata |
| | | | tctctaacatggatatcttcgagaaggttgacgctatctttgataaacat |
| | | | gaagtccaggagatcaaggagaagatcctgaacagcgactacgacgtgga |
| | | | ggatttcttcgagggtgagttcttcaattttgttttgacccaagaaggca |
| | | | tcgatgtctacaatgcgataattggtggttttgttaccgagagcggggag |
| | | | aaaattaagggtctgaacgaatatatcaacttgtacaaccagaaaaccaa |
| | | | acaaaaactgccgaagtttaagccgctgtacaagcaagtcttgtcggatc |
| | | | gtgaatcgctgagcttttacggcgaaggctacaccagcgatgaggaggtg |
| | | | ttggaggtgttccgcaatacgttgaataagaactctgaaattttttccag |
| | | | cattaagaagctggaaaaactattcaagaacttcgacgagtatagcagcg |
| | | | caggtatctttgtgaaaaacggtccggctattagcaccatctcgaaagac |
| | | | attttcggcgaatggaatgttattcgtgataagtggaatgccgaatacga |
| | | | cgacattcatctgaagaaaaaggcggttgtaactgaaaagtacgaggacg |
| | | | atcgtcgtaagtcgttcaaaaagatcggctccttctctctggagcaactg |
| | | | caagagtacgccgatgccgatttgtcagtcgtggagaagctgaaagagat |
| | | | catcattcaaaaagttgacgaaatctataaggtctatggtagcagtgaaa |
| | | | aactgtttgacgcggacttcgtgctggagaaaagcctgaagaaaaatgac |
| | | | gccgtggtggcaatcatgaaagatcttctggatagcgtcaagtctttcga |
| | | | aaactacatcaaagcgtttttcggtgaaggcaaggaaacgaatcgtgacg |
| | | | agagcttctatggtgatttcgtcctagcctatgatatcctgcttaaggtg |
| | | | gatcacatctatgatgccatccgcaactatgttacccagaaaccgtactc |
| | | | aaaggacaagttcaagttatacttccagaatccgcagtttatgggtggct |
| | | | gggataaagataaggaaaccgactatcgcgccaccattttacgttacggt |
| | | | agtaagtactatctggcaatcatggataaaaaatatgcgaaatgcctgca |
| | | | aaagatcgacaaggacgatgttaacggcaattatgagaagatcaactaca |
| | | | aactgctgccgggcccaaataagatgctgccgaaggtgtttttcagcaaa |
| | | | aaatggatggcgtattacaatccgagcgaggacatccaaaagatctacaa |
| | | | aaacggtacgtttaagaagggtgatatgttcaacctcaacgattgtcata |
| | | | aacttattgacttcttcaaggatagcatatctcgttatccgaagtggagc |
| | | | aacgcgtatgactttaactttagcgaaaccgagaaatacaaagatatcgc |
| | | | gggtttctatcgtgaagttgaagaacagggttacaaagtgtccttcgagt |
| | | | ccgcgagcaagaaggaggtagacaagctggtcgaggagggtaagttgtac |
| | | | atgtttcagatttacaataaggacttttccgacaagtcccatggtacacc |
| | | | gaatttgcacaccatgtatttcaagcttctgttcgacgagaacaaccacg |
| | | | gccagatccgtctgagcggtggtgcagagctgttcatgcgtagagctagc |
| | | | ctgaagaaagaagagttggtcgttcatccggctaattcgccgatcgcgaa |
| | | | caaaaatccagataaccctaaaaagactacgaccctgagctatgatgtgt |
| | | | ataaagacaaacgctttagcgaagaccagtatgagttacacattccgatc |
| | | | gctatcaacaagtgcccgaagaatattttcaagatcaacaccgaagtgcg |
| | | | tgttcttttgaagcacgatgacaacccgtatgtgattggtattgctcgtg |
| | | | gtgaacgtaatctgctgtacattgtggttgtggacggcaaaggaaacatc |
| | | | gtggaacagtacagcctgaacgaaatcattaacaatttcaacggcattcg |
| | | | cattaagacggattaccattctttgctggacaaaaaggagaaagagcgtt |
| | | | tcgaggcccgtcagaactggacctccatcgagaacattaaggagctgaag |
| | | | gcgggttacatcagccaggtggtgcacaaaatctgcgaactggtggagaa |
| | | | gtacgacgcggtgattgctttggcggatttgaactccggttttaagaaca |
| | | | gccgcgttaaagtggaaaagcaggtttatcagaaattcgaaaaaatgctg |
| | | | attgataaattgaattacatggttgacaaaaagtctaacccgtgtgcaac |
| | | | cggcggtgcgctgaagggctaccaaatcaccaataagtttgaaagcttta |
| | | | agagcatgagcacgcaaaatggcttcattttttacattccggcgtggctg |
| | | | accagcaagatcgatccgtcaaccggctttgttaatttactgaagacgaa |
| | | | gtatacctctattgccgactctaaaaaatttattagttccttcgatagaa |
| | | | tcatgtatgttccggaagaggacctgttcgaatttgcactggactacaag |
| | | | aactttagccgtaccgatgctgattatataaagaaatggaaactctacag |
| | | | ctatggcaaccgcatccgtatctttcgcaacccgaaaaaaaacaacgtgt |
| | | | tcgactgggaagaggtatgtctgacttcagcgtacaaggagcttttcaat |
| | | | aaatatggtatcaactaccagcagggtgatatccgtgcactgctttgcga |
| | | | gcaaagcgataaagcgttctattcaagcttcatggcgctgatgagcctga |
| | | | tgctgcaaatgcgcaacagcatcaccggtcgtaccgatgttgcatttctg |
| | | | atttctccagtaaaaaacagcgatggtatcttttacgacagccgtaatta |
| | | | cgaggcccaagaaaatgcgattttgccgaagaacgctgacgcgaatggtg |
| | | | catacaacattgcaaggaaagttctgtgggcaatcggccaatttaaaaaa |
| | | | gcggaagatgagaagctggacaaagtgaaaattgcgattagcaacaagga |
| | | | gtggctggagtatgcgcagacctccgttaaatcagggggaagtggcggaa |
| | | | gcggtgggtctacgaacctgagcgacattattgaaaaagaaactggtaaa |
| | | | cagttggtgatccaagaatccattctgatgttaccggaggaagtggaaga |
| | | | ggtgatcggcaacaagccggagtccgacatcctggttcacaccgcgtatg |
| | | | atgagtcgaccgatgagaatgttatgctgctgaccagcgacgccccagag |
| | | | tacaaaccgtgggcgctcgtcatccaggatagcaatggcgaaaacaagat |
| | | | caagatgttgtctggcggtagcatggctccgaaaaagaagcgtaaagtag |
| | | | gtattcatggtgttccggcatcaggt |
| 91 | Protospacer | AsCas12-pS | taatttctactcttgtagatgtcagcaacgacaggcagctggg |
| 92 | Protospacer | CasPhi-pS | caacgattgcccctcacgaggggacgtcagcaacgacaggcagctggg |
| 93 | Protospacer | dCas9-pS | gtcagcaacgacaggcagctgttttagagctagaaatagcaagttaaaat |
| | | | aaggctagtccgttatcaacttgaaaaagtggcaccgagtcggtgctttt |
| | | | tt |
| 94 | Protospacer | ErCas12-pS | gtcaaaagacctttttaatttctactcttgtagatgtcagcaacgacagg |
| | | | cagctggg |
| 95 | Protospacer | FnCas12-pS | aatttctactgttgtagatgtcagcaacgacaggcagctggg |
| 96 | Protospacer | LbCas12-pS | aatttctactaagtgtagatgtcagcaacgacaggcagctggg |
| 97 | Protospacer | MbCas12-pS | aatttctactgtttgtagatgtcagcaacgacaggcagctgggcgacccc |
| 98 | Functional- | XTEN-NG-N7 | ggatctggaagtgagacacccgggacttcagaatccggtctatggtgag |
| | ization | 1-48:xten | caagggcgaggaggataacatggcctctctcccagcgacacatgagtta |
| | component | 49-756:NG | cacatctttggctccatcaacggtgtggactttgacatggtgggtcaggg |
| | | 757-1011:N7 | caccggcaatccaaatgatggttatgaggagttaaacctgaagtccacca |
| | | 1012-1012:Stop | agggtgacctccagttctccccctggattctggtccctcatatcgggtat |
| | | | ggcttccatcagtacctgccctaccctgacgggatgtcgcctttccaggc |
| | | | cgccatggtagatggctccggataccaagtccatcgcacaatgcagtttg |
| | | | aagatggtgcctcccttactgttaactaccgctacacctacgagggaagc |
| | | | cacatcaaaggagaggcccaggtgaaggggactggtttccctgctgacgg |
| | | | tcctgtgatgaccaactcgctgaccgctgcggactggtgcaggtcgaaga |
| | | | agacttaccccaacgacaaaaccatcatcagtacctttaagtggagttac |
| | | | accactggaaatggcaagcgctaccggagcactgcgcggaccacctacac |
| | | | ctttgccaagccaatggcggctaactatctgaagaaccagccgatgtacg |
| | | | tgttccgtaagacggagctcaagcactccaagaccgagctcaacttcaag |
| | | | gagtggcaaaaggcctttaccgatgtgatgggcatggacgagctgtacaa |
| | | | gtcaggtgctgcagcggccgaattcaagcgtgaagaacaagcaaggaaag |
| | | | ctaaggtgaacaatgagaaaaagacggaaatagtgaaaccagagagttgt |
| | | | agcaatgaaggagatgtcaaggatctgaaaagaaaggactctgaggatgg |
| | | | aaacgagggtgaggaagaagaagcttcttcgaaaccgaaaaagccaaaag |
| | | | ttgctctttctcatcttcaggacattgacgacacagaagctgatcaagaa |
| | | | gaagagtaa |
| 99 | Promoter | pCmYLCV | tggcagacatactgtcccacaaatgaagatggaatctgtaaaagaaaacg |
| | | | cgtgaaataatgcgtctgacaaaggttaggtcggctgcctttaatcaata |
| | | | ccaaagtggtccctaccacgatggaaaaactgtgcagtcggtttggcttt |
| | | | ttctgacgaacaaataagattcgtggccgacaggtgggggtccaccatgt |
| | | | gaaggcatcttcagactccaataatggagcaatgacgtaagggcttacga |
| | | | aataagtaagggtagtttgggaaatgtccactcacccgtcagtctataaa |
| | | | tacttagcccctccctcattgttaagggagcaaaatctcagagagatagt |
| | | | cctagagagagaaagagagcaagtagcctagaagtagtcaaggcggcgaa |
| | | | gtattcaggcacgtggccaggaagaagaaaagccaagacgacgaaaacag |
| | | | gtaagagctaagctta |
| 100 | Promoter | p35S | agattagccttttcaatttcagaaagaatgctaacccacagatggttaga |
| | | | gaggcttacgcagcaagtctcatcaagacgatctacccgagcaataatct |
| | | | acaggaaatcaaataccttcccaagaaggttaaagatgcagtcaaaagat |
| | | | tcaggactaactgcatcaagaacacagagaaagatatatttctcaagatc |
| | | | agaagtactattccagtatggacgattcaaggcttgcttcacaaaccaag |
| | | | gcaagtaatagagattggagtctctaaaaaggtagttcccactgaatcaa |
| | | | aggccatggagtcaaagattcaaatagaggacctaacagaactcgccgta |
| | | | aagactggcgaacagttcatacagagtctcttacgactcaatgacaagaa |
| | | | gaaaatcttcgtcaacatggtggagcacgacacacttgtctactccaaaa |
| | | | atatcaaagatacagtctcagtagaccaaagggcaattgagacttttcaa |
| | | | caaagggtaatatccggaaacctcctcggattccattgcccagctatctg |
| | | | tcactttattgtgaagatagtggaaaaggaaggtggctcctacaaatgcc |
| | | | atcattgcgataaaggaaaggccatcgttgaagatgcctctgccgacagt |
| | | | ggtcccaaagatggacccccacccacgaggagcatcgtggaaaaagaaaa |
| | | | cgttccaaccacgtctacaaagcaagtggattgatgtgatatctccactg |
| | | | acgtaagggatgacgcacaatcccactatccttcgcaagacccttcctct |
| | | | atataaggaagttcatttcatttggagagaacacgggggactctagagga |
| | | | tccaaca |
| 101 | Functional- | pCmYLCV- | tggcagacatactgtcccacaaatgaagatggaatctgtaaaagaaaacg |
| | ized base | A3A-dCas9- | cgtgaaataatgcgtctgacaaaggttaggtcggctgcctttaatcaata |
| | editor | NG | ccaaagtggtccctaccacgatggaaaaactgtgcagtcggtttggcttt |
| | | 1-466:pCmYLCV | ttctgacgaacaaataagattcgtggccgacaggtgggggtccaccatgt |
| | | 467-5671:A3A- | gaaggcatcttcagactccaataatggagcaatgacgtaagggcttacg |
| | | dCas9 | aaataagtaagggtagtttgggaaatgtccactcacccgtcagtctata |
| | | 5672:6730:-NG | aatacttagcccctccctcattgttaagggagcaaaatctcagagagata |
| | | | gtcctagagagagaaagagagcaagtagcctagaagtagtcaaggcggcg |
| | | | aagtattcaggcacgtggccaggaagaagaaaagccaagacgacgaaaac |
| | | | aggtaagagctaagcttaatgggaagtcatcaccaccaccaccattcagg |
| | | | tgagaacctgtacttccagagcatggctccgaaaaagaaacgtaaggtgg |
| | | | gtatccacggcgttccggcgatggaagcaagtccagcttcaggacccagg |
| | | | cacctgatggacccgcatattttcacctctaattttaacaacggtattgg |
| | | | ccgtcacaagacgtacctgtgttatgaagtggaacgtcttgacaacggca |
| | | | cctccgtgaaaatggaccagcatcgtggtttcttgcataatcaagctaaa |
| | | | aacctgctgtgcggtttctacggccgtcacgcggaactgcgttttttgga |
| | | | cctggttccgagcctgcaactggacccggcacagatttatcgtgttactt |
| | | | ggttcatcagctggtcgccgtgttttagctggggttgcgccggcgaggta |
| | | | cgcgcgtttttgcaagagaacacccacgtgcgtttgcgcatcttcgccgc |
| | | | tcgtatctacgattatgacccactgtataaagaagcgctccagatgctgc |
| | | | gcgatgcaggcgcgcaggtttccatcatgacctacgacgagttcaagcac |
| | | | tgctgggatacctttgtcgatcatcagggctgcccgttccagccgtggga |
| | | | tggtctggatgagcacagccaggcgttaagcggtcgcctcagagcgattc |
| | | | tgcaaaaccaaggtaattctggaagtgagacacccgggacttcagaatcc |
| | | | gcgaccccggagtctggaatggataaaaagtattcaataggactagctat |
| | | | tggcaccaacagcgttggttgggcagttatcaccgatgaatataaggtcc |
| | | | cgagcaagaagtttaaggtcctgggcaacaccgatcgccacagcataaaa |
| | | | aagaaccttattggtgcgctgctgttcgattctggtgagactgccgaagc |
| | | | gacccgtctgaagagaaccgcgcgtcgtagatacacccggcgcaagaacc |
| | | | gtatctgctatctgcaagaaattttcagcaacgagatggctaaggtcgac |
| | | | gactccttctttcatcgcctggaggaaagctttctggttgaggaagacaa |
| | | | aaagcacgagcgtcatccgattttcggtaacattgttgatgaagttgcct |
| | | | accacgaaaaataccccacaatttatcatctgcgtaaaaaactggtagac |
| | | | tccaccgataaggcggatttacgccttatctatttggcgctggcgcatat |
| | | | gattaagttccgcggtcattttttgatcgagggcgacctgaatccggata |
| | | | acagcgacgttgacaaactgtttatccagctggtgcagacctataaccag |
| | | | ttgttcgaagagaacccgattaatgcgtctggcgtggacgcgaaggcgat |
| | | | cctgagcgctcgtctgtccaagtcacgccgcctggagaacttgatcgcgc |
| | | | aactgccgggtgagaagaaaaacggcttgttcggcaatctgattgctctg |
| | | | tccctaggtttgaccccgaatttcaaatctaattttgatctggcggaaga |
| | | | tgcgaaattacagttgagcaaggacacttacgatgacgatctggacaacc |
| | | | tgctcgctcagatcggcgatcagtatgccgatcttttcctggccgcgaag |
| | | | aacctgagcgatgccatcttgctatcggacatcttacgtgttaacaccga |
| | | | aatcaccaaagctccgctaagcgcgtccatgattaaacgttacgatgaac |
| | | | accaccaggatctgacgctgctgaaagcgttggtgcgccagcaactgccg |
| | | | gaaaaatacaaggaaattttcttcgaccaaagcaagaacggttatgcagg |
| | | | ctatatcgacggtggtgcgtcgcaagaagaattttataaattcatcaaac |
| | | | cgattttggagaagatggatggtaccgaggagctgctggtaaaattgaat |
| | | | cgggaagacctcttgcgaaagcagcgtactttcgataatggtagcatccc |
| | | | gcaccagattcatctgggcgaacttcacgcaatcctacgtcgtcaagagg |
| | | | atttctacccgtttctgaaggataaccgtgaaaagatcgagaagattctg |
| | | | acctttcgtattccgtattatgtgggtccgctggcgagaggcaacagccg |
| | | | ttttgcgtggatgacccgcaagtcggaagagactatcacgccgtggaatt |
| | | | tcgaggaggtcgtggacaagggtgcatcagcgcaatcttttattgaacgt |
| | | | atgaccaattttgataaaaaccttccaaatgaaaaagtgttgccgaagca |
| | | | cagcttgctgtacgagtatttcaccgtttacaatgaactgacgaaagtca |
| | | | agtacgttaccgaaggtatgcgtaagccagcctttctgtctggggagcaa |
| | | | aaaaaagccattgtggatctgctgttcaagaccaatcgcaaggttaccgt |
| | | | caagcaactgaaagaggattacttcaaaaaaatcgaatgttttgactccg |
| | | | tggaaatcagcggcgttgaagaccgctttaatgcatctctgggcacctac |
| | | | cacgatctactcaagatcatcaaggataaggacttcctggacaacgaaga |
| | | | aaatgaggatatcttggaagatattgtgctgactttaacgttgttcgagg |
| | | | atcgtgagatgattgaagaacgcctcaaaacgtacgcacatctgtttgac |
| | | | gataaggtgatgaaacagctaaagcgccgtcgttacaccggttggggtcg |
| | | | tctgtctagaaagctgattaacggcattcgtgacaaacaatctggcaaga |
| | | | ccatcctggacttcctcaaatccgacggcttcgcaaatcgtaatttcatg |
| | | | caactgatccatgatgatagcctgacctttaaagaagatattcagaaggc |
| | | | tcaagtgtccggccagggtgactcgctgcatgagcatatcgcgaatctgg |
| | | | ctggctcccctgcgataaaaaaaggtattctccaaacggtgaaagttgtg |
| | | | gatgaactggttaaagtcatgggtcgtcataaaccggaaaacatcgtgat |
| | | | tgagatggcacgtgagaaccagaccacccagaaaggtcaaaaaaacagtc |
| | | | gtgagcgtatgaaacgtatcgaagaggggattaaggagttaggcagccag |
| | | | attctgaaggagcacccggttgagaacacccagctgcaaaacgagaagct |
| | | | ctacctgtattacctgcaaaacggccgtgacatgtatgttgaccaggagc |
| | | | tggacatcaatcgtctcagcgactacgacgtggatgctatcgtgccgcag |
| | | | agctttctaaaagatgattccatcgacaataaagtgctcacccgtagcga |
| | | | caagaaccgtggtaaaagcgacaacgtgccgagcgaggaagtagttaaga |
| | | | aaatgaaaaactactggcgtcagctattgaacgccaagcttatcacccag |
| | | | cgtaagtttgacaacttgacgaaggcagagcgtggcggcctgagcgagct |
| | | | agacaaggcgggttttattaaacgccagctggtggaaactcgccagatta |
| | | | ccaaacacgttgcgcaaatcttggactcacgcatgaatacgaagtacgac |
| | | | gaaaacgataaattgatccgcgaggtgaaggtcataaccctgaaatctaa |
| | | | actggttagcgatttccggaaggactttcaattttataaagtccgggaga |
| | | | ttaacaactatcaccatgcacacgacgcttacttaaatgcggttgttggc |
| | | | acggccctgatcaagaagtacccgaaactggaaagcgagtttgtttatgg |
| | | | tgactacaaggtgtatgacgttcgcaagatgattgcaaaatccgaacaag |
| | | | agattggtaaggcgacggctaagtattttttctacagcaatatcatgaac |
| | | | ttcttcaagactgaaatcaccctggcgaacggtgaaattcgtaaacgtcc |
| | | | gttgatcgaaaccaacggtgaaaccggtgaaatcgtttgggataaaggta |
| | | | gggattttgcgaccgttcgtaaggtcctgagcatgccgcaggttaacatc |
| | | | gtgaaaaagaccgaagtgcagaccggcggtttctccaaggagagtatctt |
| | | | accgaagcgcaacagtgacaagctgatcgcgcgtaaaaaagactgggatc |
| | | | cgaaaaaatacggcggctttgactccccgaccgtcgcgtacagcgtactg |
| | | | gtcgtggctaaagtggagaaaggcaaatcgaaaaagctgaaatccgttaa |
| | | | agagttgctgggtattacgattatggaacgttcgagcttcgagaaaaatc |
| | | | cgatcgacttcctggaggccaaaggctacaaggaagttaaaaaagacctg |
| | | | atcattaaactgccgaagtacagcctgttcgagctcgagaacggtcgcaa |
| | | | acgcatgctggcgagcgcgggtgagctccaaaaaggcaacgaattggctc |
| | | | tgccgagcaagtacgtgaacttcctgtatctggcctcccattatgaaaaa |
| | | | ctgaagggtagcccggaagataacgaacaaaagcagctgttcgtggaaca |
| | | | acataaacactatctggacgagattattgagcaaatcagcgagttcagca |
| | | | aacgcgtgattctggcggatgctaatctggacaaggttctttcagcctat |
| | | | aataaacaccgtgataagccgatccgtgaacaggcagaaaatattattca |
| | | | cctgttcaccctgacgaacctgggtgcgccagcagcgtttaaatacttcg |
| | | | acacaacgatcgaccggaagcgttataccagcaccaaagaggttcttgac |
| | | | gccaccttgattcaccaaagcatcacgggtctgtatgaaacccgtattga |
| | | | cttgagccagttgggcggagattcagggggaagtggcggaagcggtgggt |
| | | | ctacgaacctgagcgacattattgaaaaagaaactggtaaacagttggtg |
| | | | atccaagaatccattctgatgttaccggaggaagtggaagaggtgatcgg |
| | | | caacaagccggagtccgacatcctggttcacaccgcgtatgatgagtcga |
| | | | ccgatgagaatgttatgctgctgaccagcgacgccccagagtacaaaccg |
| | | | tgggcgctcgtcatccaggatagcaatggcgaaaacaagatcaagatgtt |
| | | | gtctggcggtagcatggctccgaaaaagaagcgtaaagtaggtattcatg |
| | | | gtgttccggcatcaggttcaggttctggaagtgagacacccgggacttca |
| | | | gaatccgcgaccccggagtctatggtgagcaagggcgaggaggataacat |
| | | | ggcctctctcccagcgacacatgagttacacatctttggctccatcaacg |
| | | | gtgtggactttgacatggtgggtcagggcaccggcaatccaaatgatggt |
| | | | tatgaggagttaaacctgaagtccaccaagggtgacctccagttctcccc |
| | | | ctggattctggtccctcatatcgggtatggcttccatcagtacctgccct |
| | | | accctgacgggatgtcgcctttccaggccgccatggtagatggctccgga |
| | | | taccaagtccatcgcacaatgcagtttgaagatggtgcctcccttactgt |
| | | | taactaccgctacacctacgagggaagccacatcaaaggagaggcccagg |
| | | | tgaaggggactggtttccctgctgacggtcctgtgatgaccaactcgctg |
| | | | accgctgcggactggtgcaggtcgaagaagacttaccccaacgacaaaac |
| | | | catcatcagtacctttaagtggagttacaccactggaaatggcaagcgct |
| | | | accggagcactgcgcggaccacctacacctttgccaagccaatggcggct |
| | | | aactatctgaagaaccagccgatgtacgtgttccgtaagacggagctcaa |
| | | | gcactccaagaccgagctcaacttcaaggagtggcaaaaggcctttaccg |
| | | | atgtgatgggcatggacgagctgtacaagtcaggtgctgcagcggccgaa |
| | | | ttcaagcgtgaagaacaagcaaggaaagctaaggtgaacaatgagaaaaa |
| | | | gacggaaatagtgaaaccagagagttgtagcaatgaaggagatgtcaagg |
| | | | atctgaaaagaaaggactctgaggatggaaacgagggtgaggaagaagaa |
| | | | gcttcttcgaaaccgaaaaagccaaaagttgctctttctcatcttcagga |
| | | | cattgacgacacagaagctgatcaagaagaagagtaagcttctctagcta |
| | | | gagtcgatcgacaagctcgagtttctccataataatgtgtgagtagttcc |
| | | | cagataagggaattagggttcctatagggtttcgctcatgtgttgagcat |
| | | | ataagaaacccttagtatgtatttgtatttgtaaaatacttctatcaata |
| | | | aaatttctaattcctaaaaccaaaatccagtactaaaatccagatcgct |
| 102 | Functional- | p35S-A3A- | agattagccttttcaatttcagaaagaatgctaacccacagatggt |
| | ized base | dCas9-NG | tagagaggcttacgcagcaagtctcatcaagacgatctacccgagcaat |
| | editor | 1-857:p35S | aatctacaggaaatcaaataccttcccaagaaggttaaagatgcagtca |
| | | 858-6062:A3A- | aaagattcaggactaactgcatcaagaacacagagaaagatatatttct |
| | | dCas9 | caagatcagaagtactattccagtatggacgattcaaggcttgcttcac |
| | | 6063-7076:NG | aaaccaaggcaagtaatagagattggagtctctaaaaaggtagttcccac |
| | | | tgaatcaaaggccatggagtcaaagattcaaatagaggacctaacagaac |
| | | | tcgccgtaaagactggcgaacagttcatacagagtctcttacgactcaat |
| | | | gacaagaagaaaatcttcgtcaacatggtggagcacgacacacttgtcta |
| | | | ctccaaaaatatcaaagatacagtctcagtagaccaaagggcaattgaga |
| | | | cttttcaacaaagggtaatatccggaaacctcctcggattccattgccca |
| | | | gctatctgtcactttattgtgaagatagtggaaaaggaaggtggctccta |
| | | | caaatgccatcattgcgataaaggaaaggccatcgttgaagatgcctctg |
| | | | ccgacagtggtcccaaagatggacccccacccacgaggagcatcgtggaa |
| | | | aaagaaaacgttccaaccacgtctacaaagcaagtggattgatgtgatat |
| | | | ctccactgacgtaagggatgacgcacaatcccactatccttcgcaagacc |
| | | | cttcctctatataaggaagttcatttcatttggagagaacacgggggact |
| | | | ctagaggatccaacaatgggaagtcatcaccaccaccaccattcaggtga |
| | | | gaacctgtacttccagagcatggctccgaaaaagaaacgtaaggtgggta |
| | | | tccacggcgttccggcgatggaagcaagtccagcttcaggacccaggcac |
| | | | ctgatggacccgcatattttcacctctaattttaacaacggtattggccg |
| | | | tcacaagacgtacctgtgttatgaagtggaacgtcttgacaacggcacct |
| | | | ccgtgaaaatggaccagcatcgtggtttcttgcataatcaagctaaaaac |
| | | | ctgctgtgcggtttctacggccgtcacgcggaactgcgttttttggacct |
| | | | ggttccgagcctgcaactggacccggcacagatttatcgtgttacttggt |
| | | | tcatcagctggtcgccgtgttttagctggggttgcgccggcgaggtacgc |
| | | | gcgtttttgcaagagaacacccacgtgcgtttgcgcatcttcgccgctcg |
| | | | tatctacgattatgacccactgtataaagaagcgctccagatgctgcgcg |
| | | | atgcaggcgcgcaggtttccatcatgacctacgacgagttcaagcactgc |
| | | | tgggatacctttgtcgatcatcagggctgcccgttccagccgtgggatgg |
| | | | tctggatgagcacagccaggcgttaagcggtcgcctcagagcgattctgc |
| | | | aaaaccaaggtaattctggaagtgagacacccgggacttcagaatccgcg |
| | | | accccggagtctggaatggataaaaagtattcaataggactagctattgg |
| | | | caccaacagcgttggttgggcagttatcaccgatgaatataaggtcccga |
| | | | gcaagaagtttaaggtcctgggcaacaccgatcgccacagcataaaaaag |
| | | | aaccttattggtgcgctgctgttcgattctggtgagactgccgaagcgac |
| | | | ccgtctgaagagaaccgcgcgtcgtagatacacccggcgcaagaaccgta |
| | | | tctgctatctgcaagaaattttcagcaacgagatggctaaggtcgacgac |
| | | | tccttctttcatcgcctggaggaaagctttctggttgaggaagacaaaaa |
| | | | gcacgagcgtcatccgattttcggtaacattgttgatgaagttgcctacc |
| | | | acgaaaaataccccacaatttatcatctgcgtaaaaaactggtagactcc |
| | | | accgataaggcggatttacgccttatctatttggcgctggcgcatatgat |
| | | | taagttccgcggtcattttttgatcgagggcgacctgaatccggataaca |
| | | | gcgacgttgacaaactgtttatccagctggtgcagacctataaccagttg |
| | | | ttcgaagagaacccgattaatgcgtctggcgtggacgcgaaggcgatcct |
| | | | gagcgctcgtctgtccaagtcacgccgcctggagaacttgatcgcgcaac |
| | | | tgccgggtgagaagaaaaacggcttgttcggcaatctgattgctctgtcc |
| | | | ctaggtttgaccccgaatttcaaatctaattttgatctggcggaagatgc |
| | | | gaaattacagttgagcaaggacacttacgatgacgatctggacaacctgc |
| | | | tcgctcagatcggcgatcagtatgccgatcttttcctggccgcgaagaac |
| | | | ctgagcgatgccatcttgctatcggacatcttacgtgttaacaccgaaat |
| | | | caccaaagctccgctaagcgcgtccatgattaaacgttacgatgaacacc |
| | | | accaggatctgacgctgctgaaagcgttggtgcgccagcaactgccggaa |
| | | | aaatacaaggaaattttcttcgaccaaagcaagaacggttatgcaggcta |
| | | | tatcgacggtggtgcgtcgcaagaagaattttataaattcatcaaaccga |
| | | | ttttggagaagatggatggtaccgaggagctgctggtaaaattgaatcgg |
| | | | gaagacctcttgcgaaagcagcgtactttcgataatggtagcatcccgca |
| | | | ccagattcatctgggcgaacttcacgcaatcctacgtcgtcaagaggatt |
| | | | tctacccgtttctgaaggataaccgtgaaaagatcgagaagattctgacc |
| | | | tttcgtattccgtattatgtgggtccgctggcgagaggcaacagccgttt |
| | | | tgcgtggatgacccgcaagtcggaagagactatcacgccgtggaatttcg |
| | | | aggaggtcgtggacaagggtgcatcagcgcaatcttttattgaacgtatg |
| | | | accaattttgataaaaaccttccaaatgaaaaagtgttgccgaagcacag |
| | | | cttgctgtacgagtatttcaccgtttacaatgaactgacgaaagtcaagt |
| | | | acgttaccgaaggtatgcgtaagccagcctttctgtctggggagcaaaaa |
| | | | aaagccattgtggatctgctgttcaagaccaatcgcaaggttaccgtcaa |
| | | | gcaactgaaagaggattacttcaaaaaaatcgaatgttttgactccgtgg |
| | | | aaatcagcggcgttgaagaccgctttaatgcatctctgggcacctaccac |
| | | | gatctactcaagatcatcaaggataaggacttcctggacaacgaagaaaa |
| | | | tgaggatatcttggaagatattgtgctgactttaacgttgttcgaggatc |
| | | | gtgagatgattgaagaacgcctcaaaacgtacgcacatctgtttgacgat |
| | | | aaggtgatgaaacagctaaagcgccgtcgttacaccggttggggtcgtct |
| | | | gtctagaaagctgattaacggcattcgtgacaaacaatctggcaagacca |
| | | | tcctggacttcctcaaatccgacggcttcgcaaatcgtaatttcatgcaa |
| | | | ctgatccatgatgatagcctgacctttaaagaagatattcagaaggctca |
| | | | agtgtccggccagggtgactcgctgcatgagcatatcgcgaatctggctg |
| | | | gctcccctgcgataaaaaaaggtattctccaaacggtgaaagttgtggat |
| | | | gaactggttaaagtcatgggtcgtcataaaccggaaaacatcgtgattga |
| | | | gatggcacgtgagaaccagaccacccagaaaggtcaaaaaaacagtcgtg |
| | | | agcgtatgaaacgtatcgaagaggggattaaggagttaggcagccagatt |
| | | | ctgaaggagcacccggttgagaacacccagctgcaaaacgagaagctcta |
| | | | cctgtattacctgcaaaacggccgtgacatgtatgttgaccaggagctgg |
| | | | acatcaatcgtctcagcgactacgacgtggatgctatcgtgccgcagagc |
| | | | tttctaaaagatgattccatcgacaataaagtgctcacccgtagcgacaa |
| | | | gaaccgtggtaaaagcgacaacgtgccgagcgaggaagtagttaagaaaa |
| | | | tgaaaaactactggcgtcagctattgaacgccaagcttatcacccagcgt |
| | | | aagtttgacaacttgacgaaggcagagcgtggcggcctgagcgagctaga |
| | | | caaggcgggttttattaaacgccagctggtggaaactcgccagattacca |
| | | | aacacgttgcgcaaatcttggactcacgcatgaatacgaagtacgacgaa |
| | | | aacgataaattgatccgcgaggtgaaggtcataaccctgaaatctaaact |
| | | | ggttagcgatttccggaaggactttcaattttataaagtccgggagatta |
| | | | acaactatcaccatgcacacgacgcttacttaaatgcggttgttggcacg |
| | | | gccctgatcaagaagtacccgaaactggaaagcgagtttgtttatggtga |
| | | | ctacaaggtgtatgacgttcgcaagatgattgcaaaatccgaacaagaga |
| | | | ttggtaaggcgacggctaagtattttttctacagcaatatcatgaacttc |
| | | | ttcaagactgaaatcaccctggcgaacggtgaaattcgtaaacgtccgtt |
| | | | gatcgaaaccaacggtgaaaccggtgaaatcgtttgggataaaggtaggg |
| | | | attttgcgaccgttcgtaaggtcctgagcatgccgcaggttaacatcgtg |
| | | | aaaaagaccgaagtgcagaccggcggtttctccaaggagagtatcttacc |
| | | | gaagcgcaacagtgacaagctgatcgcgcgtaaaaaagactgggatccga |
| | | | aaaaatacggcggctttgactccccgaccgtcgcgtacagcgtactggtc |
| | | | gtggctaaagtggagaaaggcaaatcgaaaaagctgaaatccgttaaaga |
| | | | gttgctgggtattacgattatggaacgttcgagcttcgagaaaaatccga |
| | | | tcgacttcctggaggccaaaggctacaaggaagttaaaaaagacctgatc |
| | | | attaaactgccgaagtacagcctgttcgagctcgagaacggtcgcaaacg |
| | | | catgctggcgagcgcgggtgagctccaaaaaggcaacgaattggctctgc |
| | | | cgagcaagtacgtgaacttcctgtatctggcctcccattatgaaaaactg |
| | | | aagggtagcccggaagataacgaacaaaagcagctgttcgtggaacaaca |
| | | | taaacactatctggacgagattattgagcaaatcagcgagttcagcaaac |
| | | | gcgtgattctggcggatgctaatctggacaaggttctttcagcctataat |
| | | | aaacaccgtgataagccgatccgtgaacaggcagaaaatattattcacct |
| | | | gttcaccctgacgaacctgggtgcgccagcagcgtttaaatacttcgaca |
| | | | caacgatcgaccggaagcgttataccagcaccaaagaggttcttgacgcc |
| | | | accttgattcaccaaagcatcacgggtctgtatgaaacccgtattgactt |
| | | | gagccagttgggcggagattcagggggaagtggcggaagcggtgggtcta |
| | | | cgaacctgagcgacattattgaaaaagaaactggtaaacagttggtgatc |
| | | | caagaatccattctgatgttaccggaggaagtggaagaggtgatcggcaa |
| | | | caagccggagtccgacatcctggttcacaccgcgtatgatgagtcgaccg |
| | | | atgagaatgttatgctgctgaccagcgacgccccagagtacaaaccgtgg |
| | | | gcgctcgtcatccaggatagcaatggcgaaaacaagatcaagatgttgtc |
| | | | tggcggtagcatggctccgaaaaagaagcgtaaagtaggtattcatggtg |
| | | | ttccggcatcaggttcaggttctggaagtgagacacccgggacttcagaa |
| | | | tccgcgaccccggagtctatggtgagcaagggcgaggaggataacatggc |
| | | | ctctctcccagcgacacatgagttacacatctttggctccatcaacggtg |
| | | | tggactttgacatggtgggtcagggcaccggcaatccaaatgatggttat |
| | | | gaggagttaaacctgaagtccaccaagggtgacctccagttctccccctg |
| | | | gattctggtccctcatatcgggtatggcttccatcagtacctgccctacc |
| | | | ctgacgggatgtcgcctttccaggccgccatggtagatggctccggatac |
| | | | caagtccatcgcacaatgcagtttgaagatggtgcctcccttactgttaa |
| | | | ctaccgctacacctacgagggaagccacatcaaaggagaggcccaggtga |
| | | | aggggactggtttccctgctgacggtcctgtgatgaccaactcgctgacc |
| | | | gctgcggactggtgcaggtcgaagaagacttaccccaacgacaaaaccat |
| | | | catcagtacctttaagtggagttacaccactggaaatggcaagcgctacc |
| | | | ggagcactgcgcggaccacctacacctttgccaagccaatggcggctaac |
| | | | tatctgaagaaccagccgatgtacgtgttccgtaagacggagctcaagca |
| | | | ctccaagaccgagctcaacttcaaggagtggcaaaaggcctttaccgatg |
| | | | tgatgggcatggacgagctgtacaagtcaggtgctgcagcggccgaattc |
| | | | aagcgtgaagaacaagcaaggaaagctaaggtgaacaatgagaaaaagac |
| | | | ggaaatagtgaaaccagagagttgtagcaatgaaggagatgtcaaggatc |
| | | | tgaaaagaaaggactctgaggatggaaacgagggtgaggaagaagaagct |
| | | | tcttcgaaaccgaaaaagccaaaagttgctctttctcatcttcaggacat |
| | | | tgacgacacagaagctgatcaagaagaagagtaagcttctctagctagag |
| | | | tcgatcgacaagctcgagtttctccataataatgtgtgagtagttcccag |
| | | | ataagggaattagggttcctatagggtttcgctcatgtgttgagcatata |
| | | | agaaacccttagtatgtatttgtatttgtaaaatacttctatcaataaaa |
| | | | tttctaattcctaaaaccaaaatccagtactaaaatccagatcgct |

### EXAMPLES

The presently disclosed subject matter will be better understood by reference to the following Examples, which are provided as exemplary of the invention, and not by way of limitation.

### Example 1

Selection of Nucleases and Deaminases

The selection of nucleases and deaminases was conducted by performing a literature search of reported CBE base-editors. Along with other novel nucleases or deaminases not reported in their application as base-editor, a selection was performed based on their efficiency, PAM requirement, enzyme substrate and size to encompass the editing space. Sequences for all reported variants were obtained through the NCBI nucleotide sequence database and collected for further processing. Sequences for cognate deaminase fusion partners were also collected, and design considerations were determined from reported literature of functional activity. Sequences were codon-optimised using GenSmart™ Codon Optimization (GenScript).

Modular Assembly

Design of assemblies was established using a modular cloning strategy with standardised overhangs (Weber et al., 2011, A modular cloning system for standardized assembly of multigene constructs, PLoS ONE 6(2): e16765; Patron et al., 2015 “Standards for plant synthetic biology: A common syntax for exchange of DNA parts”, New Phytologist 208(1):13-19 (2015); EP2580332B1). An adaptation was made to enable CDS to be composed from 3 different modular parts by including an additional overhang situated between the deaminase and the Cas protein that enables placing serine-glycine amino acids between both modules. Using the proposed standardised overhangs, the promoter is situated between A (GGAG) and B (TACT), the 5′UTR/RBS sequence between B and C (AATG), the deaminase module between C and X (TGGA), the Cas protein between X and D (AGGT), an additional coding C-terminus module between D and E (GCTT) and finally a 3′UTR and terminator sequence between E and F (CGCT) overhangs (FIG. 2).

The Deaminase module comprises a DNA sequence encoding a His purification tag with a TEV cleavage site and a SV40 nuclear localisation sequence designed and added to the 5′ of the codon-optimised deaminases (at the N-terminus) and DNA sequence encoding a XTEN linker with additional flexible amino acids at the 3′ of the codon-optimised deaminases (at the C-terminus). The Nuclease module comprises a DNA sequence encoding a UGI, a small flexible SGGS linker and a nucleoplasmin bipartite nuclear localisation signal at the 3′ of the codon-optimized site-directed nucleases (at the C-terminus).

The vector backbones in which these modular components are situated, contain convergent flanking BsaI sites that enable release of the part for consequent directional ligation with compatible overhangs into the destination vector.

Vector Design for Detection of Activity

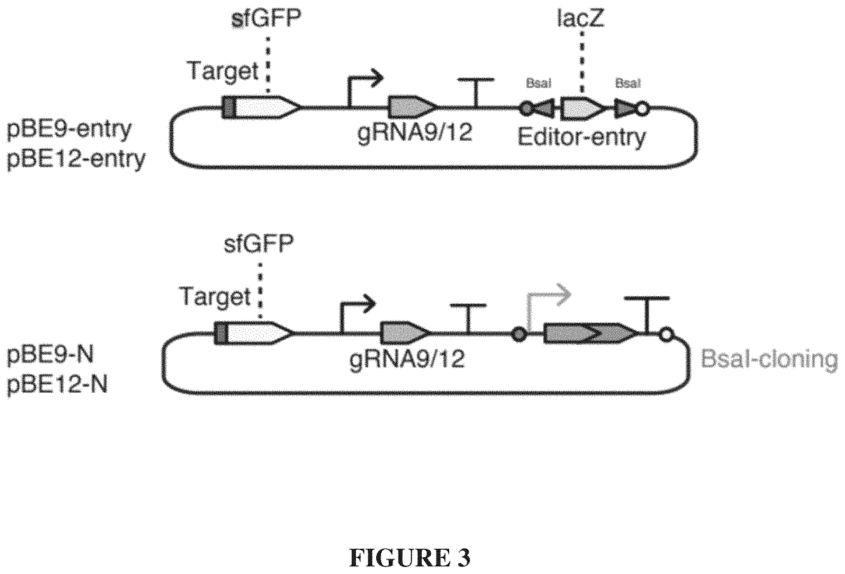

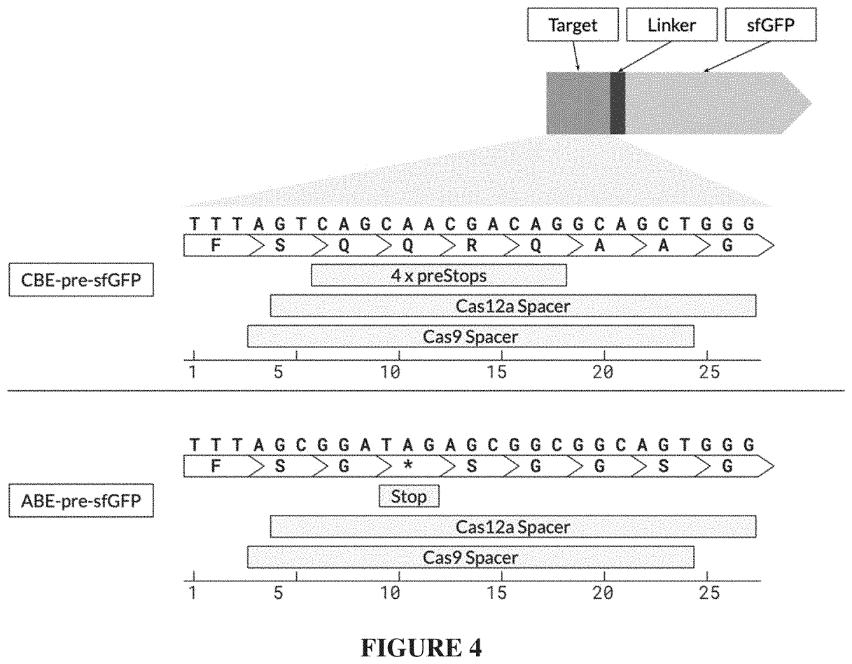

The Destination vectors (pBE-entry) were prepared in order to perform a phenotypic assay of function of cloned base-editors using a constitutively expressed fluorescent protein. These vectors contain three main components (FIG. 3):
I) Divergent BsaI restriction sites that enable cloning of base-editor cassette: promoter (AB), 5′UTR/RBS (BC), deaminase module (CX), nuclease module (XD), C-terminus module (DE) and 3′UTR and terminator sequence (EF).
II) Constitutive or regulable cassettes for expression of nuclease gRNA. AarI restriction sites in this component allow cloning of gRNA protospacer sequences to target pre-sfGFP regions for CBEs composed by Cas9-based base-editors.
III) A cassette for constitutive expression of sfGFP. Additionally, this component contains a pre-sfGFP region (5′ of sfGFP sequence) that, for CBEs, is composed of multiple putative stop codons at the 5′ section of the coding sequence for early protein termination upon CBE editing event (FIG. 4).
In vitro construction of vectors encoding editors

The three main components described before along with the deaminase and nuclease modules were synthesized by Twist Bioscience. Components of destination vectors (pBE-entry) contain SapI flanking sites. In vitro construction of these destination vectors was performed by SapI-mediated Golden Gate assembly using synthesized fragments (Twist Bioscience) and a PCR-amplified pCAodd-1 vector backbone (Pollak, B. et al., 2020, Universal loop assembly: open, efficient and cross-kingdom DNA fabrication, Synth Biol (Oxf). 5(1), ysaa001) with flanking SapI restriction sites. Cloning of base-editors to destination vectors (pBE-entry) was performed as described in Pollak et al. (2019, Loop assembly: a simple and open system for recursive fabrication of DNA circuits. New Phytologist 222(1), 628-640) by odd level (BsaI) ligation.

Detection and Characterisation of Editors

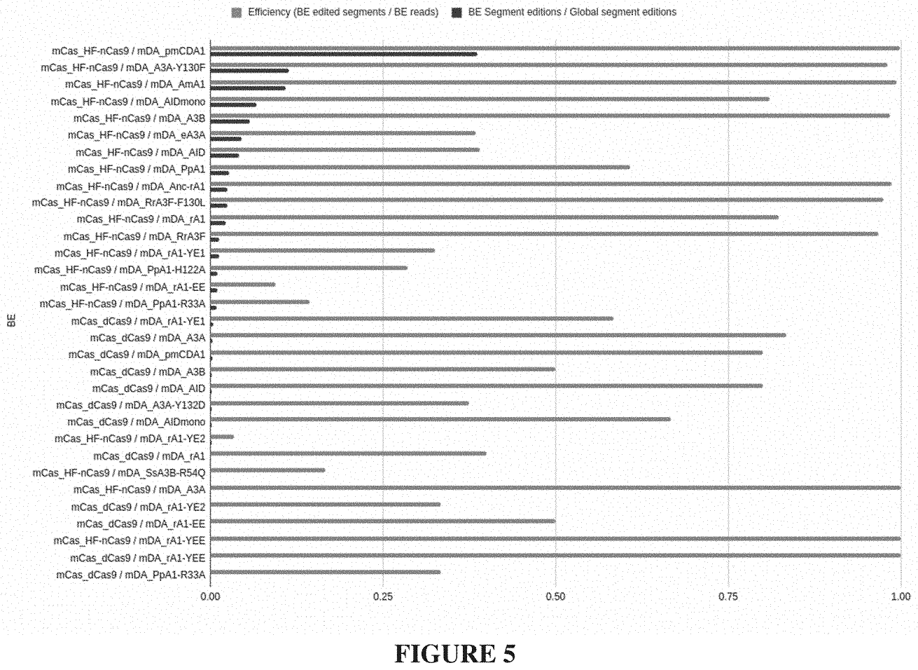

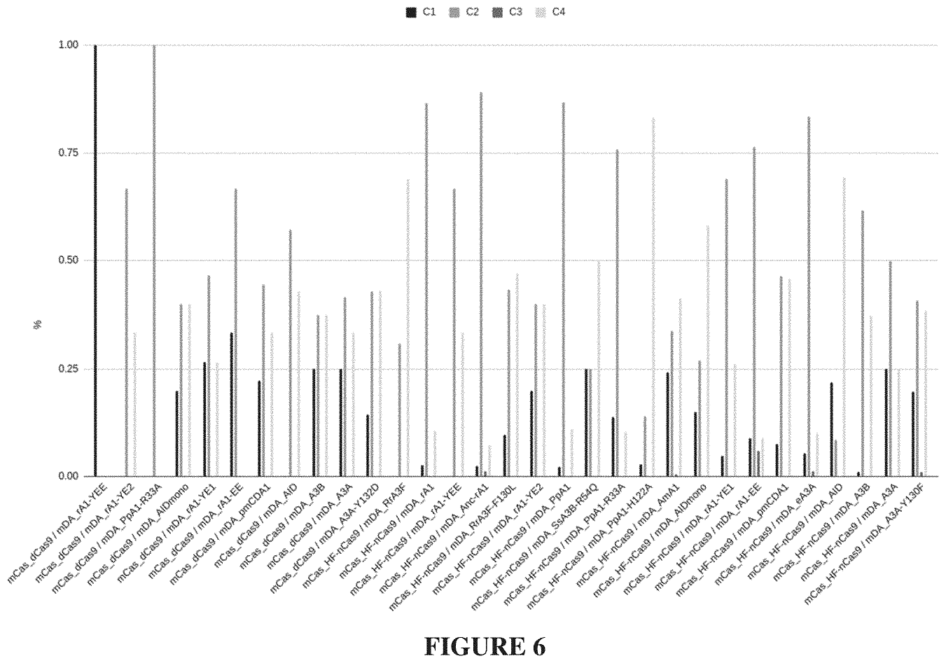

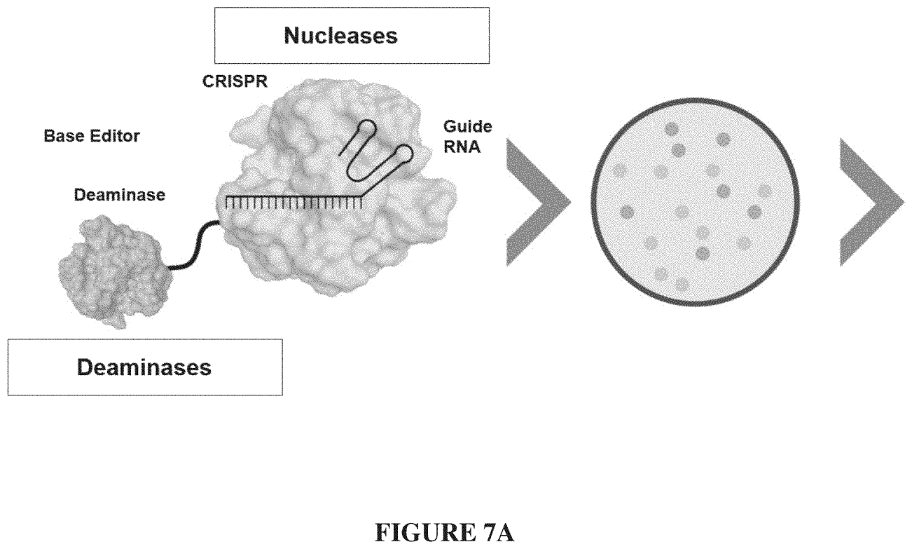

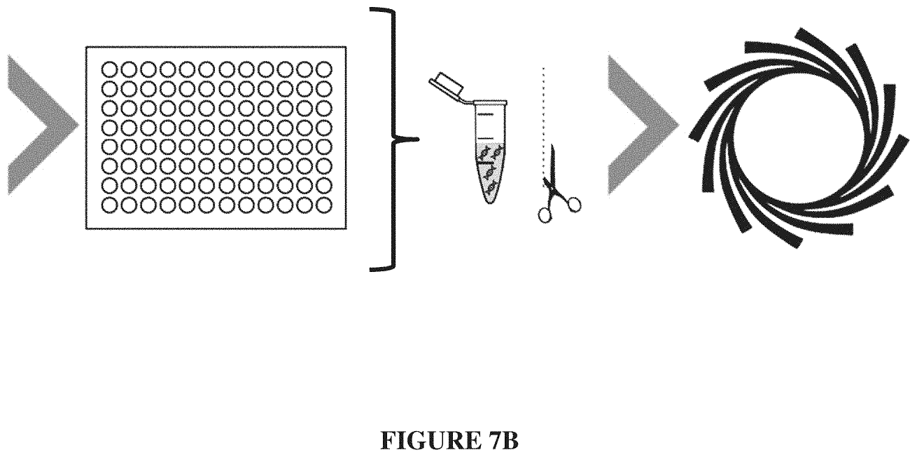

A combinatorial experiment comprising four Cas9 nuclease modules and 23 deaminase modules was performed by one-pot odd level (BsaI) ligation of base-editor cassettes as described in Pollak et al. (2019). After ligation, chemically competent TOP10 cells (no. C4040100; ThermoFisher) were transformed and plated into LB containing Kanamycin and a defined concentration of inductor for base editor expression. Edited and unedited colonies (Nonfluorescent/Fluorescent) were grown overnight in 0.5 LB/Kan ml in 2-ml 96-well plates. Fluorescent and nonfluorescent cultures were combined separately and plasmid extracted using the Promega Wizard® Plus SV Minipreps DNA Purification System and further digested by the PstI restriction enzyme. Both linear plasmid combinations were separately sequenced using Oxford Nanopore Technologies Min-lon flow cells (R.9.4.1) and Genomic DNA by Ligation (SQK-LSK109). Single reads were analyzed using bioinformatics for recognition of base editor by BLAST, detection of single edits in the pre-sfGFP region as well as generation of statistics (active base-editor combinations, number of edits for each single base-editor, efficiency estimation) (FIGS. 5 and 6). On-target, off-target, editing window (position and broad), substrate preference and processivity are the main parameters that define the performance of a base-editor. A plasmid-based platform was developed so as to enable the determination of these parameters in a high-throughput experiment by applying the modular design of the base editors assembly, target multiplexing, analysis by sequencing and bioinformatics.

Expression and Production of Editors

Vector for expression of A3A (deaminase)+HFnCas9 (nuclease) base-editor under regulation of pBAD promoter were assembled as described in Pollak et al. (2019) by odd level (BsaI) ligation of: AB_pBAD (promoter)+BC_B0034m (RBS)+CX_A3A (deaminase module)+XD_HFnCas9 (nuclease module)+DE_3×Stop (translation stop codons)+EF_B0015 (Terminator) to a pCAodd-1 vector backbone. Ligation product was transformed to chemically competent TOP10 cells (no. C4040100; ThermoFisher). Protein expression was induced by adding 0.1% Arabinose (final concentration) to a 1:100 inoculated LB liquid medium grown at 25° C. for 16h. Total protein was extracted using B-PER (Bacterial Protein Extraction Reagent, no.78243; ThermoFisher), purified using HisPur Cobalt Resin (no.89964; ThermoFisher) and concentrated and buffer exchanged using Pierce concentrators (no.88523; ThermoFisher).

### Example 2

Assembled Base Editors

Following the methodology described in Example 1, the following base editors were assembled using nuclease dCas9: A3A/dCas9, Anc-rA1/dCas9, eA3A/dCas9, evoCDA1/dCas9, minStart/dCas9, mTadA8e/dCas9, rA1/dCas9, rA1-YE1/dCas9; using nuclease dCasPhi2: A3A/dCasPhi2, A3A-Y130F/dCasPhi2, eA3A/dCasPhi2, evoCDA1/dCasPhi2, minStart/dCasPhi2, mTadA8e/dCasPhi2, rA1/dCasPhi2, rA1-YE1/dCasPhi2; and using nuclease dLbCas12a: A3A/dLbCas12a, A3B/dLbCas12a, AID/dLbCas12a, AmA1/dLbCas12a, evoAPOBEC1/dLbCas12a, evoFERNY/dLbCas12a, hA3G-CTD/dLbCas12a, minStart/dLbCas12a, pmCDA1/dLbCas12a, PpA1-R33A/dLbCas12a, rA1-YEE/dLbCas12a, RrA3F-F130L/dLbCas12a, SsA3B-R54Q/dLbCas12a. The base editors were assembled as follows, from N-terminus to C-terminus: 6×His/TEV-Cleavage site/SV40-NLS/Deaminase/XTEN-Linker/Site-directed nuclease/SGGS-Linker/UGI/Nucleoplasmin-NLS. minStart/Site-directed nuclease was used as a negative control for the nucleotide-modifying enzyme module, which lacks a deaminase and consists of, from N-terminus to C-terminus, 6×His/TEV-Cleavage site/SV40-NLS/XTEN-Linker/Site-directed nuclease/SGGS-Linker/UGI/Nucleoplasmin-NLS. minStart was used as a negative control for the nucleotide-modifying enzyme module, which lacks a deaminase and consists of, from N-terminus to C-terminus, 6×His/TEV-Cleavage site/SV40-NLS/XTEN-Linker. All these base editors were characterized in terms of overall performance and editing window.

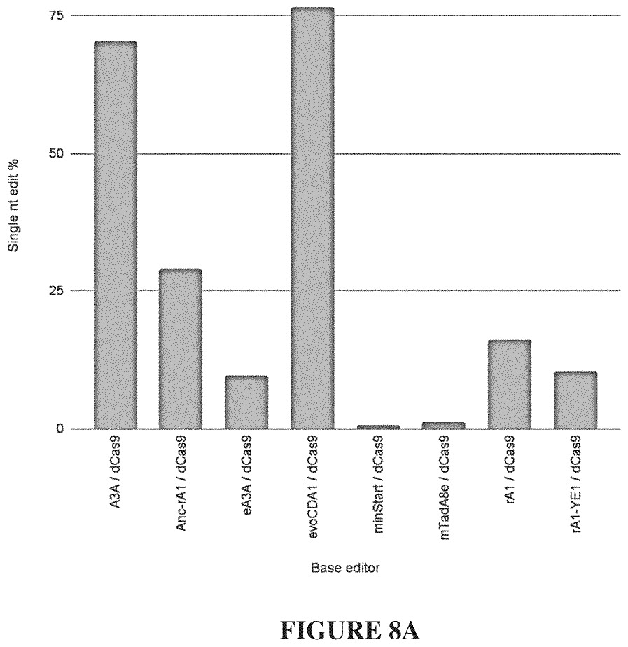

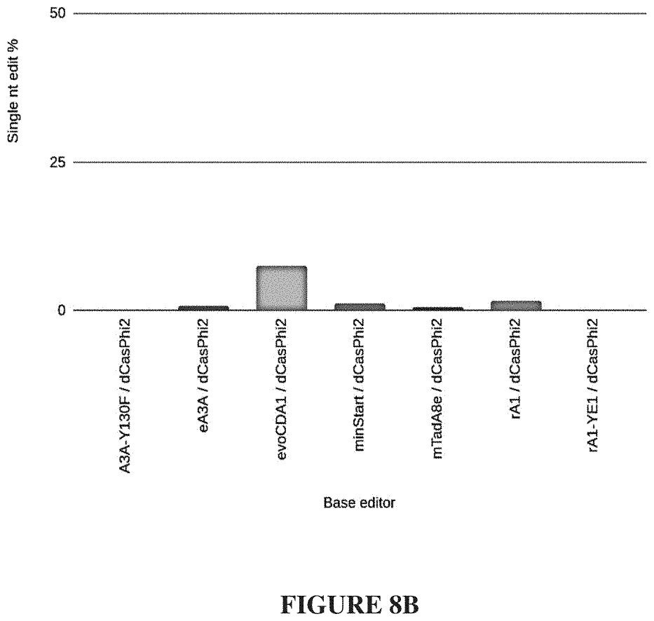

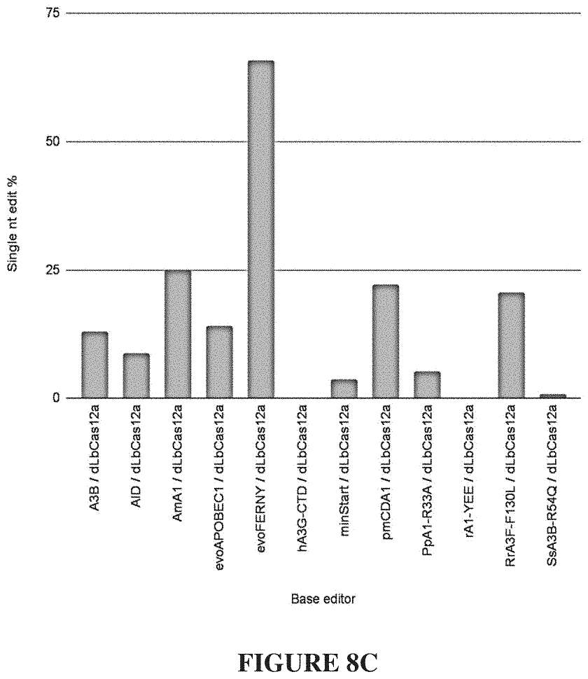

Overall performances of these assembled base editors are depicted in FIGS. 8A-8C. In the case of editors based on dCas9, all editors were able to perform editing at any of the 4 sites within the pre-sfGFP sequence, reaching a maximum with deaminase evoCDA1 and a minimum with the negative control for the deaminase function, minStart (FIG. 8A). On the other hand, editing with editors based on nucleases Cas12a or CasPhi2 was dependent on the deaminase with which these editors were assembled. In the case of Cas12a editors, a maximum percentage of editing was performed when using deaminase evoFERNY, nevertheless when using deaminases hA3G-CTD or rA1-YEE no editing was observed (FIG. 8B). In the case of CasPhi2 editors, they could perform editing on nucleotide sites, but with rates far below those observed with the other editors. When these editors were assembled with deaminases A3A-Y130F or rA1-YE1, no editing was observed (FIG. 8C).

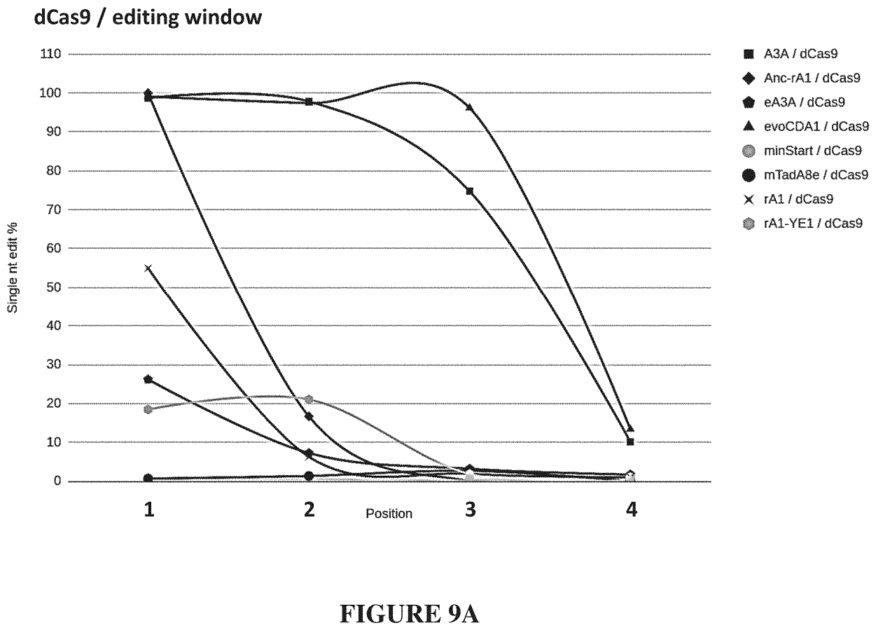

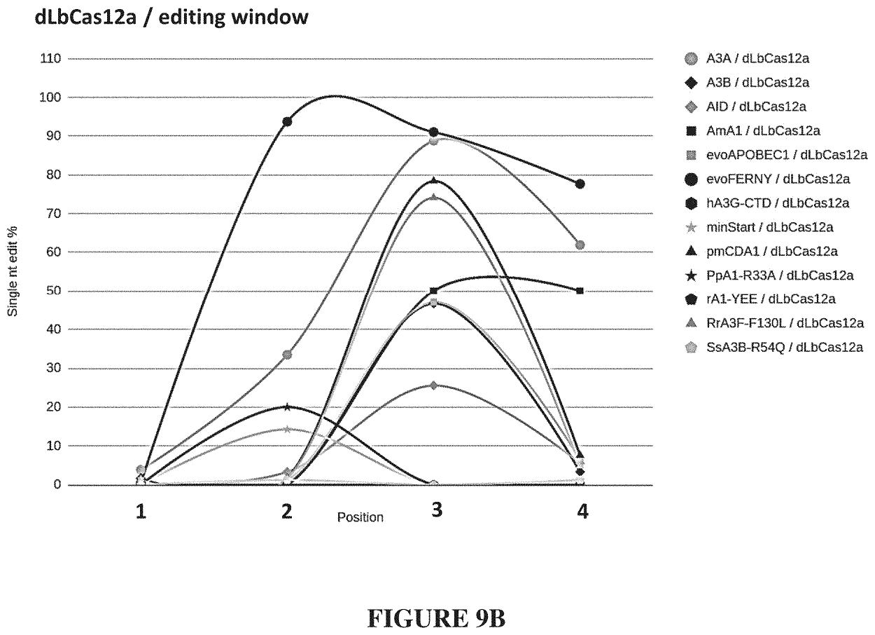

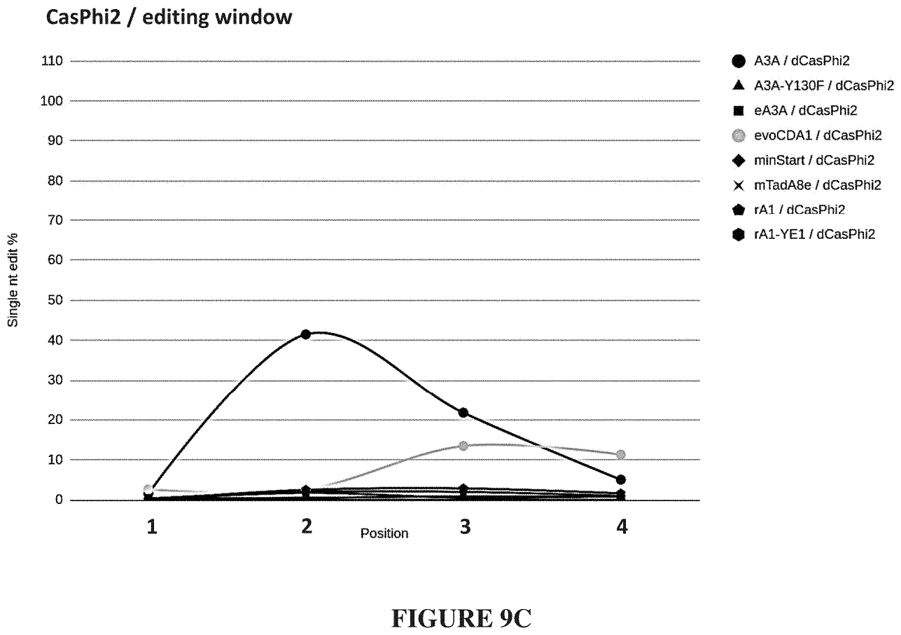

As depicted in FIGS. 9A-9C, the assembled base editors showed different optimal editing windows. In this case, the editing was performed on the pre-sfGFP region of the assemblies which contains 4 potential sites (per sgRNA), labeled as positions 1-4 in FIGS. 9A-9C. The optimal position is position 1 when using dCas9, but if this nuclease is assembled together with evoCDA1 or A3A deaminases, positions 2 and 3 can be optimal positions as well (FIG. 9A). This means that in the case of dCas9 base editors the target is preferentially edited at sites far from the PAM site of the sgRNA. Regarding the base editors based on nuclease Cas12a, optimal site was reported to be position 3, however for deaminases evoFERNY, pmCDA1 and RrA3F-F130L, the optimal site was position 2 (FIG. 9B). In the case of editors based on nuclease CasPhi2, significant editing was observed when using deaminases A3A or evoCDA1, wherein the optimal sites were position 2 and position 3 of the pre-sfGFP sequence, respectively (FIG. 9C).

### Example 3

Functionalization of Base Editors

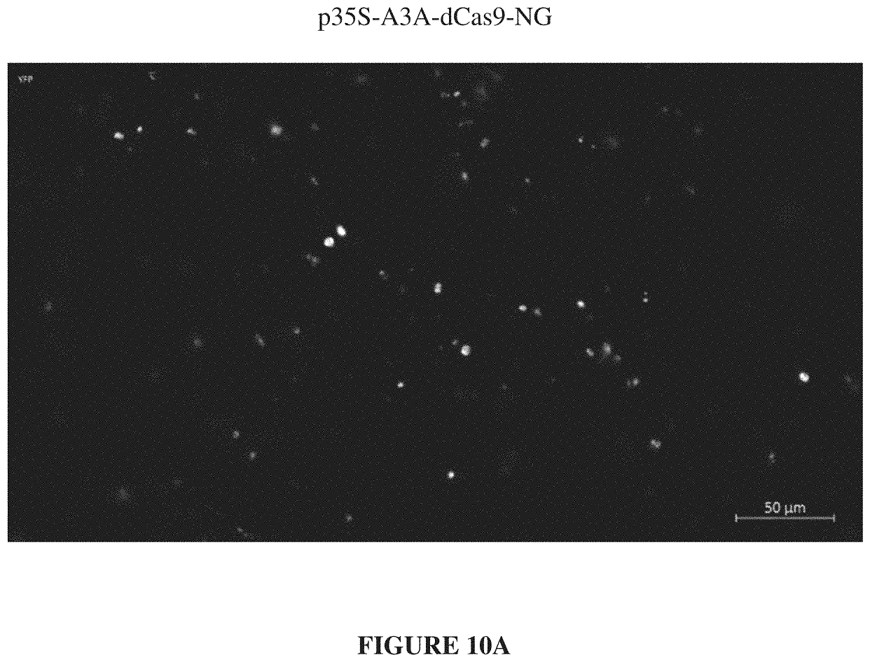

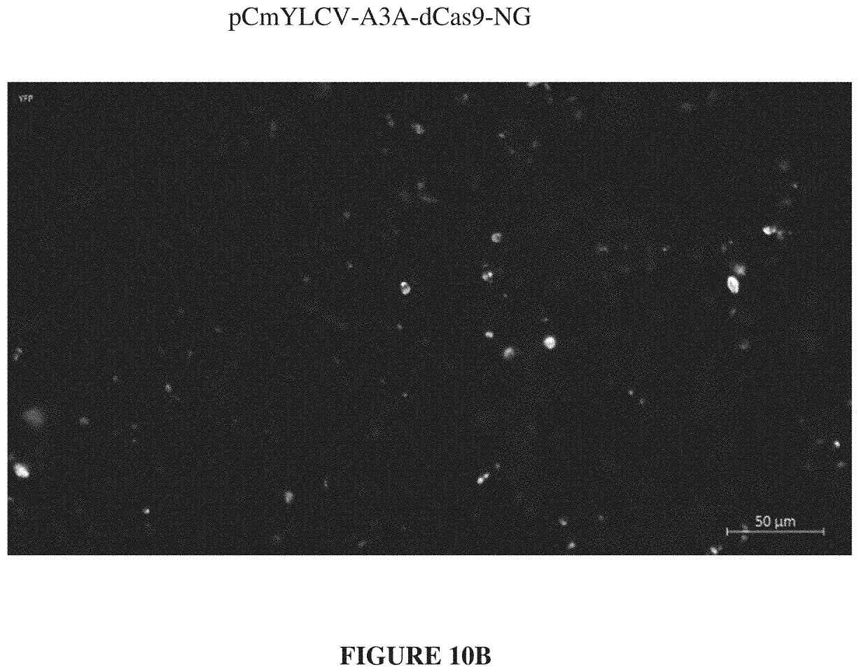

As part of the invention herein disclosed, the methodology comprises the assembly of base editors together with additional functional components to enable the detection of the presence and/or the functioning of the editor in a cell or tissue context. FIGS. 10A-10B present an example of the ability of the methodology to provide such functionalization capabilities. In this case, the base editor (A3A-dCas9) was assembled together with a fluorescent protein (mNeonGreen/NG) as part of the C-terminal of the assembly. The respective vector contained the assembled base editor and its components under the regulation of promoters p35S (FIG. 10A) or pCmYLCV (FIG. 10B). In order to verify the expression as well as localization of the base editor, the vector was introduced into Nicotiana benthamiana tissue using Agrobacterium tumefaciens transformation. Fluorescent cells or tissue showed that the introduced vector bearing the assembled editor was being transcribed and the produced editor was localized to the nucleus, hence accounting for the expression of the editor as well. This example shows that assemblies and methods described herein are able to provide the ability to add functional components accounting for the presence and/or functioning of the assembled base editors.

Embodiments of the methods and/or systems disclosed herein can include every combination and permutation of the various system components and the various method processes, including any variants (e.g. embodiments, variations, examples, specific examples, figures, etc.), where portions of embodiments of the method and/or processes described herein can be performed asynchronously (e.g. sequentially), concurrently (e.g. in parallel), or in any other suitable order by and/or using one or more instances, elements, components of, and/or other aspects of the systems and/or other entities described herein.

Any of the variants described herein (e.g., embodiments, variations, examples, specific examples, figures, etc.) and/or any portion of the variants described herein can be additionally or alternatively combined, aggregated, excluded, used, performed serially, performed in parallel, and/or otherwise applied.

Portions of embodiments of the methods and/or systems can be embodied and/or implemented at least in part as a machine configured to receive a computer-readable medium storing computer-readable instructions. The instructions can be executed by computer-executable components that can be integrated with the system. The computer-readable medium can be stored on any suitable computer-readable media such as RAMs, ROMs, flash memory, EEPROMs, optical devices (CD or DVD), hard drives, floppy drives, or any suitable device. The computer-executable component can be a general or application specific processor, but any suitable dedicated hardware or hardware/firmware combination device can alternatively or additionally execute the instructions.

As a person skilled in the art will recognize from the previous detailed description and from the figures, modifications and changes can be made to embodiments of the methods, systems, and/or variants without departing from the scope of this invention.

### ABBREVIATIONS

6×His: Polypeptide composed of 6 Histidines

A3A: DNA cytidine deaminase APOBEC3A

AID: Activation-induced cytidine deaminase

APOBEC: Apolipoprotein B mRNA editing enzyme (cytidine deaminase)

BE: Base editor

BLAST: Basic Local Alignment Search Tool

Cas protein: CRISPR associated protein (RNA-guided DNA endonuclease enzyme)

CBE: Cytidine base editor

CDS: Coding sequence

DNA: Deoxyribonucleic Acid

NLS: Nuclear localization signal or sequence

RNA: Ribonucleic Acid

sfGFP: Superfolder GFP (green fluorescent protein)

sgRNA: Single guide RNA

TALEN: Transcription activator-like effector nuclease

UTR: Untranslated region (of a transcript)

What is claimed is:

1. A method for obtaining assemblies of site-directed nucleases and nucleotide-modifying enzymes, the method comprising:
a. providing a library of modular nucleic acid sequences comprising
i. two or more site-directed nuclease modules each comprising a nucleic acid sequence encoding a different site-directed nuclease,
ii. two or more nucleotide-modifying enzyme modules each comprising a nucleic acid sequence encoding a different nucleotide-modifying enzyme, and
b. constructing two or more nucleic acid assemblies in vitro, wherein each nucleic acid assembly is constructed by joining a site-directed nuclease module with a nucleotide-modifying enzyme module to generate an nucleic acid assembly that encodes the site-directed nuclease and the nucleotide-modifying enzyme operably linked to express as a single polypeptide;
c. expressing the two or more nucleic acid assemblies constructed in step (b) to produce the site-directed nucleases and the nucleotide-modifying enzymes as single polypeptides; and
d. detecting a nucleotide-modifying activity for each of the single polypeptides expressed in step (c).

2. The method of claim 1, further comprising:
e. analyzing each nucleotide-modifying activity detected to select one or more optimal nucleic acid assemblies.

3. The method of claim 1, wherein:
i. the site directed nucleases comprise one or more Cas nucleases, one or more Cas nickases, one or more dead Cas nickases, or any combination thereof; and/or
ii. the nucleotide-modifying enzymes comprise one or more deaminases, one or more demethylases, one or more methyltransferases, or any combination thereof.

4. The method of claim 1, wherein:
i. the site-directed nucleases are each independently selected from the group consisting of AsCas12a, Cas12a (Cpf1), Cas12b, Cas12e (CasX), Cas12j, Cas12f, Cas13a, Cas13b, Cas13d, Cas14a, Cas9, dCas9, Cas9-NGv1, CasX, CasY, CasΦ, CasΦ-2 (CasPhi2), dCasΦ-2 (dCasPhi2), CjCas9, eCas9, enAsCas12a, enAsCas12a-HF1, enLbCas12a, ErCas12a, eSpCas9, eSpCas9-1.1, FnCas12a, FnCas9, HF-nCAS9, HF2-CAS9, HypaCas9, LbCas12a, d(LbCas12a), MbCas12a, NmCas9, SaCas9, SaurCas9, SauriCas9, ScCas9, SpCas9, SpCas9-HF1, SpCas9-NG, SpG, SpRY, SpyoCas9, St1Cas9, St3Cas9, StCas9, ttLbCas12a, xCas9, xCas9(3.6), and xCas9(3.7); and/or
ii. the nucleotide-modifying enzymes are selected from the group consisting of APOBEC, APOBEC-like enzymes, AID, AID-like enzymes, A3A, DNMT3A, MQ3, LSD1, RrA3F, RrA3F-F130L, AmA1, SsA3B, SsA3B-R54Q, PpA1, PpA1-H122A, PpA1-R33A, Anc-rA1, rA1, AID, pmCDA1, A3A, eA3A, rA1-YE1, rA1-EE, rA1-YE2, rA1-YEE, A3A-Y130F, A3A-Y132D, A3B, AIDmono, TadA-7.10, CGBE1, hA3G-CTD, evoAPOBEC1, evoCDA1, evoFERNY, TadA-8e, TadA-9, minStart, and mTadA8e.

5. The method of claim 1, wherein step (b) comprises:
(i) constructing a combinatorial library of nucleic acid assemblies in vitro;
(ii) constructing a plurality of nucleic acid assemblies in parallel in a single reaction; and/or
(iii) constructing two or more destination vectors, each comprising one of the nucleic acid assemblies.

6. The method claim 5, wherein constructing the two or more destination vectors comprises mixing two or more nucleic acid molecules each comprising one of the site-directed nuclease modules, one of the nucleotide-modifying enzyme modules, one of the additional modules, a protein-coding sequence, a regulatory sequence, or any combination thereof to produce the destination vectors.

7. The method of claim 6, wherein the nucleic acid molecules comprise:
i. one or more donor vectors each comprising one of the site-directed nuclease modules or one of the nucleotide-modifying enzyme modules; and/or
ii. one or more receiver vectors each comprising one of the site-directed nuclease modules or one of the nucleotide-modifying enzyme modules.

8. The method of claim 5, wherein each destination vector further comprises a first promoter, a terminator, or a combination thereof, each operably linked to the nucleic acid assembly for expression of the nucleic acid assembly.

9. The method of claim 5, wherein the destination vector further comprises a gRNA expression cassette comprising a gRNA sequence operably linked to a second promoter, a target-reporter expression cassette, or a combination thereof.

10. The method of claim 9, wherein the target-reporter expression cassette comprises a target sequence and a reporter sequence operably linked to a third promoter, wherein the target sequence and the reporter sequence are operably linked to express as a single reporter polypeptide, wherein the target sequence comprises one or more target nucleotides for editing, wherein editing of the one or more target nucleotides results in the formation of an edited target sequence having one or more stop codons, and wherein editing of the one or more target nucleotides results in the non-expression of the reporter polypeptide.

11. The method of claim 10, wherein step (e) further comprises:
i. identifying the modified sites in each of the edited target sequences;
ii. determining the sequence context of the modified sites in each of the edited target sequences; and/or
iii. identifying the window of modification in each of the target sequences.

12. A method of selecting an optimal nucleic acid assembly for editing a target nucleotide, comprising the steps of claim 2.

13. A library of nucleic acid assemblies of site-directed nucleases and nucleotide-modifying enzymes, comprising
i. two or more site-directed nuclease modules each comprising a nucleic acid sequence encoding a different site-directed nuclease, and
ii. two or more nucleotide-modifying enzyme modules each comprising a nucleic acid sequence encoding a different nucleotide-modifying enzyme;
wherein each nucleic acid assembly comprises a site-directed nuclease module and a nucleotide-modifying enzyme module, wherein each nucleic acid assembly encodes the site-directed nuclease, the nucleotide-modifying enzyme.

14. The library of claim 13, wherein:
i. the site directed nucleases comprise one or more Cas nucleases, one or more Cas nickases, one or more dead Cas nickases, or any combination thereof; and/or
ii. the nucleotide-modifying enzymes comprise one or more deaminases, one or more demethylases, one or more methyltransferases, or any combination thereof.

15. The library of claim 13, wherein:
i. the site-directed nucleases are each independently selected from the group consisting of AsCas12a, Cas12a (Cpf1), Cas12b, Cas12e (CasX), Cas12j, Cas12f, Cas13a, Cas13b, Cas13d, Cas14a, Cas9, dCas9, Cas9-NGv1, CasX, CasY, CasΦ, CasΦ-2 (CasPhi2), dCasΦ-2 (dCasPhi2), CjCas9, eCas9, enAsCas12a, enAsCas12a-HF1, enLbCas12a, ErCas12a, eSpCas9, eSpCas9-1.1, FnCas12a, FnCas9, HF-nCAS9, HF2-CAS9, HypaCas9, LbCas12a, dLbCas12a, MbCas12a, NmCas9, SaCas9, SaurCas9, SauriCas9, ScCas9, SpCas9, SpCas9-HF1, SpCas9-NG, SpG, SpRY, SpyoCas9, St1Cas9, St3Cas9, StCas9, ttLbCas12a, xCas9, xCas9(3.6), and xCas9(3.7); and/or
ii. the nucleotide-modifying enzymes are selected from the group consisting of APOBEC, APOBEC-like enzymes, AID, AID-like enzymes, A3A, DNMT3A, MQ3, LSD1, RrA3F, RrA3F-F130L, AmA1, SsA3B, SsA3B-R54Q, PpA1, PpA1-H122A, PpA1-R33A, Anc-rA1, rA1, AID, pmCDA1, A3A, eA3A, rA1-YE1, rA1-EE, rA1-YE2, rA1-YEE, A3A-Y130F, A3A-Y132D, A3B, AIDmono, TadA-7.10, CGBE1, hA3G-CTD, evoAPOBEC1, evoCDA1, evoFERNY, TadA-8e, TadA-9, minStart, and mTadA8e.

16. A base editor for editing a target nucleotide, wherein the base editor comprises:
i. a site-directed nuclease module comprising a site-directed nuclease selected from the group consisting of AsCas12a, Cas12a (Cpf1), Cas12b, Cas12e (CasX), Cas12j, Cas12f, Cas13a, Cas13b, Cas13d, Cas14a, Cas9, dCas9, Cas9-NGv1, CasX, CasY, CasΦ, CasΦ-2 (CasPhi2), dCasΦ-2 (dCasPhi2), CjCas9, eCas9, enAsCas12a, enAsCas12a-HF1, enLbCas12a, ErCas12a, eSpCas9, eSpCas9-1.1, FnCas12a, FnCas9, HF-nCAS9, HF2-CAS9, HypaCas9, LbCas12a, dLbCas12a, MbCas12a, NmCas9, SaCas9, SaurCas9, SauriCas9, ScCas9, SpCas9, SpCas9-HF1, SpCas9-NG, SpG, SpRY, SpyoCas9, St1Cas9, St3Cas9, StCas9, ttLbCas12a, xCas9, xCas9(3.6), and xCas9(3.7); and
ii. a nucleotide-modifying enzyme module comprising a nucleotide-modifying enzyme selected from the group consisting of APOBEC, APOBEC-like enzymes, AID, AID-like enzymes, A3A, DNMT3A, MQ3, LSD1, RrA3F, RrA3F-F130L, AmA1, SsA3B, SsA3B-R54Q, PpA1, PpA1-H122A, PpA1-R33A, Anc-rA1, rA1, AID, pmCDA1, A3A, eA3A, rA1-YE1, rA1-EE, rA1-YE2, rA1-YEE, A3A-Y130F, A3A-Y132D, A3B, AIDmono, TadA-7.10, CGBE1, hA3G-CTD, evoAPOBEC1, evoCDA1, evoFERNY, TadA-8e, TadA-9, minStart, and mTadA8e;
wherein the site-directed nuclease module and the nucleotide-modifying enzyme module are fused as a single polypeptide.

17. The base editor of claim 16, wherein:
i. the site-directed nuclease module comprises a site-directed nuclease selected from the group consisting of dCas9, dLbCas12a (dCpf1), and CasΦ-2 (CasPhi2); and/or
ii. the nucleotide-modifying enzyme module comprises a nucleotide-modifying enzyme selected from the group consisting of A3A, Anc-rA1, eA3A, evoCDA1, minStart, mTadA8e, rA1, rA1-YE1, A3A-Y130F, A3B, AID, AmA1, evoAPOBEC1, evoFERNY, hA3G-CTD, pmCDA1, PpA1-R33A, rA1-YEE, RrA3F-F130L, and SsA3B-R54Q.

18. The base editor of claim 16, wherein:
(1) the site-directed nuclease module comprises dCas9 and the nucleotide-modifying enzyme module comprises A3A;
(2) the site-directed nuclease module comprises dCas9 and the nucleotide-modifying enzyme module comprises Anc-rA1;
(3) the site-directed nuclease module comprises dCas9 and the nucleotide-modifying enzyme module comprises eA3A;
(4) the site-directed nuclease module comprises dCas9 and the nucleotide-modifying enzyme module comprises evoCDA1;
(5) the site-directed nuclease module comprises dCas9 and the nucleotide-modifying enzyme module comprises minStart;
(6) the site-directed nuclease module comprises dCas9 and the nucleotide-modifying enzyme module comprises mTadA8e;
(7) the site-directed nuclease module comprises dCas9 and the nucleotide-modifying enzyme module comprises rA1;
(8) the site-directed nuclease module comprises dCas9 and the nucleotide-modifying enzyme module comprises rA1-YE1;
(9) the site-directed nuclease module comprises dCasΦ-2 (dCasPhi2) and the nucleotide-modifying enzyme module comprises A3A;
(10) the site-directed nuclease module comprises dCasΦ-2 (dCasPhi2) and the nucleotide-modifying enzyme module comprises A3A-Y130F;
(11) the site-directed nuclease module comprises dCasΦ-2 (dCasPhi2) and the nucleotide-modifying enzyme module comprises eA3A;
(12) the site-directed nuclease module comprises dCasΦ-2 (dCasPhi2) and the nucleotide-modifying enzyme module comprises evoCDA1;
(13) the site-directed nuclease module comprises dCasΦ-2 (dCasPhi2) and the nucleotide-modifying enzyme module comprises minStart;
(14) the site-directed nuclease module comprises dCasΦ-2 (dCasPhi2) and the nucleotide-modifying enzyme module comprises mTadA8e;
(15) the site-directed nuclease module comprises dCasΦ-2 (dCasPhi2) and the nucleotide-modifying enzyme module comprises rA1;
(16) the site-directed nuclease module comprises dCasΦ-2 (dCasPhi2) and the nucleotide-modifying enzyme module comprises rA1-YE1;
(17) the site-directed nuclease module comprises dLbCas12a and the nucleotide-modifying enzyme module comprises A3A;
(18) the site-directed nuclease module comprises dLbCas12a and the nucleotide-modifying enzyme module comprises A3B;
(19) the site-directed nuclease module comprises dLbCas12a and the nucleotide-modifying enzyme module comprises AID;
(20) the site-directed nuclease module comprises dLbCas12a and the nucleotide-modifying enzyme module comprises AmA1;
(21) the site-directed nuclease module comprises dLbCas12a and the nucleotide-modifying enzyme module comprises evoAPOBEC1;
(22) the site-directed nuclease module comprises dLbCas12a and the nucleotide-modifying enzyme module comprises evoFERNY;
(23) the site-directed nuclease module comprises dLbCas12a and the nucleotide-modifying enzyme module comprises hA3G-CTD;
(24) the site-directed nuclease module comprises dLbCas12a and the nucleotide-modifying enzyme module comprises minStart;
(25) the site-directed nuclease module comprises dLbCas12a and the nucleotide-modifying enzyme module comprises pmCDA1;
(26) the site-directed nuclease module comprises dLbCas12a and the nucleotide-modifying enzyme module comprises PpA1-R33A;
(27) the site-directed nuclease module comprises dLbCas12a and the nucleotide-modifying enzyme module comprises rA1-YEE;
(28) the site-directed nuclease module comprises dLbCas12a and the nucleotide-modifying enzyme module comprises RrA3F-F130L; or
(29) the site-directed nuclease module comprises dLbCas12a and the nucleotide-modifying enzyme module comprises SsA3B-R54Q.

19. The base editor of claim 16, wherein the base editor comprises one or more additional modules comprising one or more additional polypeptides comprising a polypeptide for detecting the presence or the expression of the base editor in a cell or in tissue.

20. The base editor of claim 16, wherein the one or more additional polypeptides comprise a fluorescent protein or a reporter protein.


SEQUENCE LISTING (102 sequences)

SEQ ID NO: 1 - DNA, 5202 bp, synthetic construct
  source 1..5202: mol_type = other DNA
atgggaagtcatcaccaccaccaccattcaggtgagaacctgtacttccagagcatggctccgaaaaagaaacgtaaggtgggtatccacggcgttccggcgatggaagcaagtccagcttcaggacccaggcacctgatggacccgcatattttcacctctaattttaacaacggtattggccgtcacaagacgtacctgtgttatgaagtggaacgtcttgacaacggcacctccgtgaaaatggaccagcatcgtggtttcttgcataatcaagctaaaaacctgctgtgcggtttctacggccgtcacgcggaactgcgttttttggacctggttccgagcctgcaactggacccggcacagatttatcgtgttacttggttcatcagctggtcgccgtgttttagctggggttgcgccggcgaggtacgcgcgtttttgcaagagaacacccacgtgcgtttgcgcatcttcgccgctcgtatctacgattatgacccactgtataaagaagcgctccagatgctgcgcgatgcaggcgcgcaggtttccatcatgacctacgacgagttcaagcactgctgggatacctttgtcgatcatcagggctgcccgttccagccgtgggatggtctggatgagcacagccaggcgttaagcggtcgcctcagagcgattctgcaaaaccaaggtaattctggaagtgagacacccgggacttcagaatccgcgaccccggagtctggaatggataaaaagtattcaataggactagctattggcaccaacagcgttggttgggcagttatcaccgatgaatataaggtcccgagcaagaagtttaaggtcctgggcaacaccgatcgccacagcataaaaaagaaccttattggtgcgctgctgttcgattctggtgagactgccgaagcgacccgtctgaagagaaccgcgcgtcgtagatacacccggcgcaagaaccgtatctgctatctgcaagaaattttcagcaacgagatggctaaggtcgacgactccttctttcatcgcctggaggaaagctttctggttgaggaagacaaaaagcacgagcgtcatccgattttcggtaacattgttgatgaagttgcctaccacgaaaaataccccacaatttatcatctgcgtaaaaaactggtagactccaccgataaggcggatttacgccttatctatttggcgctggcgcatatgattaagttccgcggtcattttttgatcgagggcgacctgaatccggataacagcgacgttgacaaactgtttatccagctggtgcagacctataaccagttgttcgaagagaacccgattaatgcgtctggcgtggacgcgaaggcgatcctgagcgctcgtctgtccaagtcacgccgcctggagaacttgatcgcgcaactgccgggtgagaagaaaaacggcttgttcggcaatctgattgctctgtccctaggtttgaccccgaatttcaaatctaattttgatctggcggaagatgcgaaattacagttgagcaaggacacttacgatgacgatctggacaacctgctcgctcagatcggcgatcagtatgccgatcttttcctggccgcgaagaacctgagcgatgccatcttgctatcggacatcttacgtgttaacaccgaaatcaccaaagctccgctaagcgcgtccatgattaaacgttacgatgaacaccaccaggatctgacgctgctgaaagcgttggtgcgccagcaactgccggaaaaatacaaggaaattttcttcgaccaaagcaagaacggttatgcaggctatatcgacggtggtgcgtcgcaagaagaattttataaattcatcaaaccgattttggagaagatggatggtaccgaggagctgctggtaaaattgaatcgggaagacctcttgcgaaagcagcgtactttcgataatggtagcatcccgcaccagattcatctgggcgaacttcacgcaatcctacgtcgtcaagaggatttctacccgtttctgaaggataaccgtgaaaagatcgagaagattctgacctttcgtattccgtattatgtgggtccgctggcgagaggcaacagccgttttgcgtggatgacccgcaagtcggaagagactatcacgccgtggaatttcgaggaggtcgtggacaagggtgcatcagcgcaatcttttattgaacgtatgaccaattttgataaaaaccttccaaatgaaaaagtgttgccgaagcacagcttgctgtacgagtatttcaccgtttacaatgaactgacgaaagtcaagtacgttaccgaaggtatgcgtaagccagcctttctgtctggggagcaaaaaaaagccattgtggatctgctgttcaagaccaatcgcaaggttaccgtcaagcaactgaaagaggattacttcaaaaaaatcgaatgttttgactccgtggaaatcagcggcgttgaagaccgctttaatgcatctctgggcacctaccacgatctactcaagatcatcaaggataaggacttcctggacaacgaagaaaatgaggatatcttggaagatattgtgctgactttaacgttgttcgaggatcgtgagatgattgaagaacgcctcaaaacgtacgcacatctgtttgacgataaggtgatgaaacagctaaagcgccgtcgttacaccggttggggtcgtctgtctagaaagctgattaacggcattcgtgacaaacaatctggcaagaccatcctggacttcctcaaatccgacggcttcgcaaatcgtaatttcatgcaactgatccatgatgatagcctgacctttaaagaagatattcagaaggctcaagtgtccggccagggtgactcgctgcatgagcatatcgcgaatctggctggctcccctgcgataaaaaaaggtattctccaaacggtgaaagttgtggatgaactggttaaagtcatgggtcgtcataaaccggaaaacatcgtgattgagatggcacgtgagaaccagaccacccagaaaggtcaaaaaaacagtcgtgagcgtatgaaacgtatcgaagaggggattaaggagttaggcagccagattctgaaggagcacccggttgagaacacccagctgcaaaacgagaagctctacctgtattacctgcaaaacggccgtgacatgtatgttgaccaggagctggacatcaatcgtctcagcgactacgacgtggatgctatcgtgccgcagagctttctaaaagatgattccatcgacaataaagtgctcacccgtagcgacaagaaccgtggtaaaagcgacaacgtgccgagcgaggaagtagttaagaaaatgaaaaactactggcgtcagctattgaacgccaagcttatcacccagcgtaagtttgacaacttgacgaaggcagagcgtggcggcctgagcgagctagacaaggcgggttttattaaacgccagctggtggaaactcgccagattaccaaacacgttgcgcaaatcttggactcacgcatgaatacgaagtacgacgaaaacgataaattgatccgcgaggtgaaggtcataaccctgaaatctaaactggttagcgatttccggaaggactttcaattttataaagtccgggagattaacaactatcaccatgcacacgacgcttacttaaatgcggttgttggcacggccctgatcaagaagtacccgaaactggaaagcgagtttgtttatggtgactacaaggtgtatgacgttcgcaagatgattgcaaaatccgaacaagagattggtaaggcgacggctaagtattttttctacagcaatatcatgaacttcttcaagactgaaatcaccctggcgaacggtgaaattcgtaaacgtccgttgatcgaaaccaacggtgaaaccggtgaaatcgtttgggataaaggtagggattttgcgaccgttcgtaaggtcctgagcatgccgcaggttaacatcgtgaaaaagaccgaagtgcagaccggcggtttctccaaggagagtatcttaccgaagcgcaacagtgacaagctgatcgcgcgtaaaaaagactgggatccgaaaaaatacggcggctttgactccccgaccgtcgcgtacagcgtactggtcgtggctaaagtggagaaaggcaaatcgaaaaagctgaaatccgttaaagagttgctgggtattacgattatggaacgttcgagcttcgagaaaaatccgatcgacttcctggaggccaaaggctacaaggaagttaaaaaagacctgatcattaaactgccgaagtacagcctgttcgagctcgagaacggtcgcaaacgcatgctggcgagcgcgggtgagctccaaaaaggcaacgaattggctctgccgagcaagtacgtgaacttcctgtatctggcctcccattatgaaaaactgaagggtagcccggaagataacgaacaaaagcagctgttcgtggaacaacataaacactatctggacgagattattgagcaaatcagcgagttcagcaaacgcgtgattctggcggatgctaatctggacaaggttctttcagcctataataaacaccgtgataagccgatccgtgaacaggcagaaaatattattcacctgttcaccctgacgaacctgggtgcgccagcagcgtttaaatacttcgacacaacgatcgaccggaagcgttataccagcaccaaagaggttcttgacgccaccttgattcaccaaagcatcacgggtctgtatgaaacccgtattgacttgagccagttgggcggagattcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 2 - DNA, 3363 bp, synthetic construct
  source 1..3363: mol_type = other DNA
atgggaagtcatcaccaccaccaccattcaggtgagaacctgtacttccagagcatggctccgaaaaagaaacgtaaggtgggtatccacggcgttccggcgatggaagcaagtccagcttcaggacccaggcacctgatggacccgcatattttcacctctaattttaacaacggtattggccgtcacaagacgtacctgtgttatgaagtggaacgtcttgacaacggcacctccgtgaaaatggaccagcatcgtggtttcttgcataatcaagctaaaaacctgctgtgcggtttctacggccgtcacgcggaactgcgttttttggacctggttccgagcctgcaactggacccggcacagatttatcgtgttacttggttcatcagctggtcgccgtgttttagctggggttgcgccggcgaggtacgcgcgtttttgcaagagaacacccacgtgcgtttgcgcatcttcgccgctcgtatctacgattatgacccactgtataaagaagcgctccagatgctgcgcgatgcaggcgcgcaggtttccatcatgacctacgacgagttcaagcactgctgggatacctttgtcgatcatcagggctgcccgttccagccgtgggatggtctggatgagcacagccaggcgttaagcggtcgcctcagagcgattctgcaaaaccaaggtaattctggaagtgagacacccgggacttcagaatccgcgaccccggagtctggaatgccaaaacccgctgtagagtcagaattttcaaaagttttgaaaaagcactttccgggagaacgtttccgctcgagctatatgaaaagaggcggtaagatcctggctgcccaaggcgaagaggcagttgttgcgtatctacagggcaagtccgaggaggagcctccgaacttccagccaccggcgaagtgccacgtggtgaccaaatcccgtgacttcgcggaatggccgatcatgaaggcgtcggaagcgattcaacgttatatctatgcactgtcgaccaccgaacgcgcggcctgtaaaccgggcaaaagtagtgagagccatgcagcgtggttcgccgctaccggtgtcagcaaccacggctacagccacgttcagggtctgaacctgatttttgatcataccttgggccgttacgatggtgttctgaaaaaggtccaactgcgcaacgaaaaggcgagagcgcgtttagagagcattaacgcgtctagagcagatgaaggtttaccggagattaaagccgaggaggaagaagtggctacgaacgaaaccggccacctgttgcaaccgccgggtatcaatccgagcttttacgtgtatcagaccattagcccgcaggcataccgtccgcgtgacgagatcgtgctgccgccggagtacgcaggttacgtacgtgacccgaatgctccgattccgctgggtgtggtgcgtaaccgttgcgatatccaaaaagggtgcccgggctacatcccggaatggcagcgtgaagcaggtaccgcgatcagcccgaaaacaggcaaggccgtaactgtgccgggcctgtctccgaagaaaaacaaacgtatgcgtaggtactggcgctctgagaaggagaaagctcaagatgcgctgttggttaccgtacgcattggtaccgattgggttgtcatcgacgtgcgcggtctgttgcgcaacgcccgttggcgcaccatcgctcccaaggacatcagcctgaatgcgctcctcgatttgtttaccggtgatccggtgatcgatgtgcgtcgcaatattgttacctttacgtacaccctcgacgcctgtggtacctacgcccgtaaatggaccttgaaaggcaaacagacgaaagcgaccctggacaaactgacggccactcagacggttgcgttggttgcgattgcgcttggtcagaccaacccgatttccgctggcattagccgtgttacacaagaaaatggtgcactgcaatgcgaaccgctggaccggttcaccctgccggatgacctgttgaaggacatcagcgcatatcgcattgcttgggacagaaacgaagaggaactacgcgctcgcagcgtcgaggccctgccggaggcgcaacaagcggaggtccgtgcgctggacggggtgtccaaagaaaccgcgcgtacccaactgtgcgcagatttcggcctggacccgaaacgtctgccgtgggataaaatgagctccaatactacctttatcagcgaagccttgctgtctaacagcgtgagccgtgatcaggtttttttcactccggctccgaagaagggtgctaagaaaaaagcgcctgttgaagttatgcgtaaagaccgcacttgggcacgtgcgtacaagccgcgtctgagcgtcgaagctcagaaactgaagaacgaagccctgtgggcacttaagcgcacctccccggagtatttgaagctgagccgtcgcaaagaggagctgtgtcgtcgtagcatcaactatgttatcgaaaagacccgtcgccgtacgcaatgtcagattgtgatcccggtgatcgaggatctgaatgttcgcttctttcatggttctggtaagagactgcccggttgggataatttcttcaccgctaaaaaagagaaccgttggttcatccagggcctgcacaaagcgtttagcgatctgcgtacgcaccgttccttctatgtttttgaagttcgtccggagcgcacctctattacctgtccaaagtgcggtcattgcgaggtgggtaatcgtgacggtgaggcgtttcagtgcctgagctgcggcaagacgtgcaacgcagatcttgacgtcgcgacccataatctgacccaagtggcgttgaccggtaagaccatgccgaagcgcgaggagccgcgtgacgctcagggtacagcgccagcgcgcaagactaagaaggcgagcaaatcaaaggcgccaccggcggagcgcgaagaccagaccccggcgcaagagccaagccagaccagttctggaggtagcgggggtagcggtggtagcactaacttaagtgatattatcgagaaagaaaccgggaaacagctggtgatccaagaaagcattctgatgctgcccgaagaagttgaagaagtgattggtaacaagcctgagtcggacatcttagtgcacaccgcgtacgatgaaagcacggatgagaatgtgatgctgctcacctctgatgcccccgaatacaagccgtgggctctggtgatccaggactctaacggagaaaacaaaattaagatgctctctggcggctccatggcgcctaaaaaaaaacgcaaagtggggattcacggggttccggcgtaa

SEQ ID NO: 3 - DNA, 4779 bp, synthetic construct
  source 1..4779: mol_type = other DNA
atgggaagtcatcaccaccaccaccattcaggtgagaacctgtacttccagagcatggctccgaaaaagaaacgtaaggtgggtatccacggcgttccggcgatggaagcaagtccagcttcaggacccaggcacctgatggacccgcatattttcacctctaattttaacaacggtattggccgtcacaagacgtacctgtgttatgaagtggaacgtcttgacaacggcacctccgtgaaaatggaccagcatcgtggtttcttgcataatcaagctaaaaacctgctgtgcggtttctacggccgtcacgcggaactgcgttttttggacctggttccgagcctgcaactggacccggcacagatttatcgtgttacttggttcatcagctggtcgccgtgttttagctggggttgcgccggcgaggtacgcgcgtttttgcaagagaacacccacgtgcgtttgcgcatcttcgccgctcgtatctacgattatgacccactgtataaagaagcgctccagatgctgcgcgatgcaggcgcgcaggtttccatcatgacctacgacgagttcaagcactgctgggatacctttgtcgatcatcagggctgcccgttccagccgtgggatggtctggatgagcacagccaggcgttaagcggtcgcctcagagcgattctgcaaaaccaaggtaattctggaagtgagacacccgggacttcagaatccgcgaccccggagtctggaatgtcaaaactagaaaagtttacaaattgttactccctgtcgaaaaccctgagattcaaagcgatcccggtgggtaaaactcaagagaacattgataacaaacgtctgctggttgaggacgaaaagcgcgcggaggattacaaaggcgttaaaaagctgttggaccgttattacctgtcctttattaatgatgttctgcacagcattaaattaaagaacctaaacaattacatctccctcttccgtaaaaagacccgcaccgaaaaggaaaataaagaactggagaaccttgagattaatctgcgtaaagaaatcgctaaagcgtttaaaggcaacgagggttataaatccttatttaaaaaagacatcatcgaaacgattctcccagaattcctggacgacaaagacgagatcgcgctggttaattccttcaacggctttaccaccgcattcaccggcttttttgataaccgcgaaaacatgttctctgaagaggccaaaagcacctcaattgcgtttcgctgcattaacgaaaatctgacgcgttatatctctaacatggatatcttcgagaaggttgacgctatctttgataaacatgaagtccaggagatcaaggagaagatcctgaacagcgactacgacgtggaggatttcttcgagggtgagttcttcaattttgttttgacccaagaaggcatcgatgtctacaatgcgataattggtggttttgttaccgagagcggggagaaaattaagggtctgaacgaatatatcaacttgtacaaccagaaaaccaaacaaaaactgccgaagtttaagccgctgtacaagcaagtcttgtcggatcgtgaatcgctgagcttttacggcgaaggctacaccagcgatgaggaggtgttggaggtgttccgcaatacgttgaataagaactctgaaattttttccagcattaagaagctggaaaaactattcaagaacttcgacgagtatagcagcgcaggtatctttgtgaaaaacggtccggctattagcaccatctcgaaagacattttcggcgaatggaatgttattcgtgataagtggaatgccgaatacgacgacattcatctgaagaaaaaggcggttgtaactgaaaagtacgaggacgatcgtcgtaagtcgttcaaaaagatcggctccttctctctggagcaactgcaagagtacgccgatgccgatttgtcagtcgtggagaagctgaaagagatcatcattcaaaaagttgacgaaatctataaggtctatggtagcagtgaaaaactgtttgacgcggacttcgtgctggagaaaagcctgaagaaaaatgacgccgtggtggcaatcatgaaagatcttctggatagcgtcaagtctttcgaaaactacatcaaagcgtttttcggtgaaggcaaggaaacgaatcgtgacgagagcttctatggtgatttcgtcctagcctatgatatcctgcttaaggtggatcacatctatgatgccatccgcaactatgttacccagaaaccgtactcaaaggacaagttcaagttatacttccagaatccgcagtttatgggtggctgggataaagataaggaaaccgactatcgcgccaccattttacgttacggtagtaagtactatctggcaatcatggataaaaaatatgcgaaatgcctgcaaaagatcgacaaggacgatgttaacggcaattatgagaagatcaactacaaactgctgccgggcccaaataagatgctgccgaaggtgtttttcagcaaaaaatggatggcgtattacaatccgagcgaggacatccaaaagatctacaaaaacggtacgtttaagaagggtgatatgttcaacctcaacgattgtcataaacttattgacttcttcaaggatagcatatctcgttatccgaagtggagcaacgcgtatgactttaactttagcgaaaccgagaaatacaaagatatcgcgggtttctatcgtgaagttgaagaacagggttacaaagtgtccttcgagtccgcgagcaagaaggaggtagacaagctggtcgaggagggtaagttgtacatgtttcagatttacaataaggacttttccgacaagtcccatggtacaccgaatttgcacaccatgtatttcaagcttctgttcgacgagaacaaccacggccagatccgtctgagcggtggtgcagagctgttcatgcgtagagctagcctgaagaaagaagagttggtcgttcatccggctaattcgccgatcgcgaacaaaaatccagataaccctaaaaagactacgaccctgagctatgatgtgtataaagacaaacgctttagcgaagaccagtatgagttacacattccgatcgctatcaacaagtgcccgaagaatattttcaagatcaacaccgaagtgcgtgttcttttgaagcacgatgacaacccgtatgtgattggtattgctcgtggtgaacgtaatctgctgtacattgtggttgtggacggcaaaggaaacatcgtggaacagtacagcctgaacgaaatcattaacaatttcaacggcattcgcattaagacggattaccattctttgctggacaaaaaggagaaagagcgtttcgaggcccgtcagaactggacctccatcgagaacattaaggagctgaaggcgggttacatcagccaggtggtgcacaaaatctgcgaactggtggagaagtacgacgcggtgattgctttggcggatttgaactccggttttaagaacagccgcgttaaagtggaaaagcaggtttatcagaaattcgaaaaaatgctgattgataaattgaattacatggttgacaaaaagtctaacccgtgtgcaaccggcggtgcgctgaagggctaccaaatcaccaataagtttgaaagctttaagagcatgagcacgcaaaatggcttcattttttacattccggcgtggctgaccagcaagatcgatccgtcaaccggctttgttaatttactgaagacgaagtatacctctattgccgactctaaaaaatttattagttccttcgatagaatcatgtatgttccggaagaggacctgttcgaatttgcactggactacaagaactttagccgtaccgatgctgattatataaagaaatggaaactctacagctatggcaaccgcatccgtatctttcgcaacccgaaaaaaaacaacgtgttcgactgggaagaggtatgtctgacttcagcgtacaaggagcttttcaataaatatggtatcaactaccagcagggtgatatccgtgcactgctttgcgagcaaagcgataaagcgttctattcaagcttcatggcgctgatgagcctgatgctgcaaatgcgcaacagcatcaccggtcgtaccgatgttgcatttctgatttctccagtaaaaaacagcgatggtatcttttacgacagccgtaattacgaggcccaagaaaatgcgattttgccgaagaacgctgacgcgaatggtgcatacaacattgcaaggaaagttctgtgggcaatcggccaatttaaaaaagcggaagatgagaagctggacaaagtgaaaattgcgattagcaacaaggagtggctggagtatgcgcagacctccgttaaatcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 4 - DNA, 5202 bp, synthetic construct
  source 1..5202: mol_type = other DNA
atgggaagtcatcaccaccaccaccattcaggtgagaacctgtacttccagagcatggctccgaaaaagaaacgtaaggtgggtatccacggcgttccggcgatggaagcatcaccagctagtggacccaggcacctgatggatccgcacatcttcacctccaattttaataacggcatcggccgtcataagacgtacctgtgctatgaggttgaacgtttggacaacggtactagcgtcaagatggaccagcatcgtggtttccttcacaaccaagcgaaaaatctgctgtgcggtttttatggtcgtcacgccgaactgagattcttggatctggttccgtctctgcaactggacccggcgcagatttatcgtgtgacctggttcatcagctggtcgccgtgctttagctggggttgcgccggtgaagtgcgcgcatttctccaggagaacacccatgttcgtttgcgcatcttcgcggcacgtatcttcgattacgacccgctgtacaaggaggcgctgcaaatgttgcgtgatgctggcgctcaagttagcattatgacctacgacgaatttaaacactgttgggataccttcgtggatcatcagggctgtccgtttcagccatgggacggcctggacgagcacagccaggcgttatccggtcgcctgcgcgcgattctgcaaaaccagggaaactctggaagtgagacacccgggacttcagaatccgcgaccccggagtctggaatggataaaaagtattcaataggactagctattggcaccaacagcgttggttgggcagttatcaccgatgaatataaggtcccgagcaagaagtttaaggtcctgggcaacaccgatcgccacagcataaaaaagaaccttattggtgcgctgctgttcgattctggtgagactgccgaagcgacccgtctgaagagaaccgcgcgtcgtagatacacccggcgcaagaaccgtatctgctatctgcaagaaattttcagcaacgagatggctaaggtcgacgactccttctttcatcgcctggaggaaagctttctggttgaggaagacaaaaagcacgagcgtcatccgattttcggtaacattgttgatgaagttgcctaccacgaaaaataccccacaatttatcatctgcgtaaaaaactggtagactccaccgataaggcggatttacgccttatctatttggcgctggcgcatatgattaagttccgcggtcattttttgatcgagggcgacctgaatccggataacagcgacgttgacaaactgtttatccagctggtgcagacctataaccagttgttcgaagagaacccgattaatgcgtctggcgtggacgcgaaggcgatcctgagcgctcgtctgtccaagtcacgccgcctggagaacttgatcgcgcaactgccgggtgagaagaaaaacggcttgttcggcaatctgattgctctgtccctaggtttgaccccgaatttcaaatctaattttgatctggcggaagatgcgaaattacagttgagcaaggacacttacgatgacgatctggacaacctgctcgctcagatcggcgatcagtatgccgatcttttcctggccgcgaagaacctgagcgatgccatcttgctatcggacatcttacgtgttaacaccgaaatcaccaaagctccgctaagcgcgtccatgattaaacgttacgatgaacaccaccaggatctgacgctgctgaaagcgttggtgcgccagcaactgccggaaaaatacaaggaaattttcttcgaccaaagcaagaacggttatgcaggctatatcgacggtggtgcgtcgcaagaagaattttataaattcatcaaaccgattttggagaagatggatggtaccgaggagctgctggtaaaattgaatcgggaagacctcttgcgaaagcagcgtactttcgataatggtagcatcccgcaccagattcatctgggcgaacttcacgcaatcctacgtcgtcaagaggatttctacccgtttctgaaggataaccgtgaaaagatcgagaagattctgacctttcgtattccgtattatgtgggtccgctggcgagaggcaacagccgttttgcgtggatgacccgcaagtcggaagagactatcacgccgtggaatttcgaggaggtcgtggacaagggtgcatcagcgcaatcttttattgaacgtatgaccaattttgataaaaaccttccaaatgaaaaagtgttgccgaagcacagcttgctgtacgagtatttcaccgtttacaatgaactgacgaaagtcaagtacgttaccgaaggtatgcgtaagccagcctttctgtctggggagcaaaaaaaagccattgtggatctgctgttcaagaccaatcgcaaggttaccgtcaagcaactgaaagaggattacttcaaaaaaatcgaatgttttgactccgtggaaatcagcggcgttgaagaccgctttaatgcatctctgggcacctaccacgatctactcaagatcatcaaggataaggacttcctggacaacgaagaaaatgaggatatcttggaagatattgtgctgactttaacgttgttcgaggatcgtgagatgattgaagaacgcctcaaaacgtacgcacatctgtttgacgataaggtgatgaaacagctaaagcgccgtcgttacaccggttggggtcgtctgtctagaaagctgattaacggcattcgtgacaaacaatctggcaagaccatcctggacttcctcaaatccgacggcttcgcaaatcgtaatttcatgcaactgatccatgatgatagcctgacctttaaagaagatattcagaaggctcaagtgtccggccagggtgactcgctgcatgagcatatcgcgaatctggctggctcccctgcgataaaaaaaggtattctccaaacggtgaaagttgtggatgaactggttaaagtcatgggtcgtcataaaccggaaaacatcgtgattgagatggcacgtgagaaccagaccacccagaaaggtcaaaaaaacagtcgtgagcgtatgaaacgtatcgaagaggggattaaggagttaggcagccagattctgaaggagcacccggttgagaacacccagctgcaaaacgagaagctctacctgtattacctgcaaaacggccgtgacatgtatgttgaccaggagctggacatcaatcgtctcagcgactacgacgtggatgctatcgtgccgcagagctttctaaaagatgattccatcgacaataaagtgctcacccgtagcgacaagaaccgtggtaaaagcgacaacgtgccgagcgaggaagtagttaagaaaatgaaaaactactggcgtcagctattgaacgccaagcttatcacccagcgtaagtttgacaacttgacgaaggcagagcgtggcggcctgagcgagctagacaaggcgggttttattaaacgccagctggtggaaactcgccagattaccaaacacgttgcgcaaatcttggactcacgcatgaatacgaagtacgacgaaaacgataaattgatccgcgaggtgaaggtcataaccctgaaatctaaactggttagcgatttccggaaggactttcaattttataaagtccgggagattaacaactatcaccatgcacacgacgcttacttaaatgcggttgttggcacggccctgatcaagaagtacccgaaactggaaagcgagtttgtttatggtgactacaaggtgtatgacgttcgcaagatgattgcaaaatccgaacaagagattggtaaggcgacggctaagtattttttctacagcaatatcatgaacttcttcaagactgaaatcaccctggcgaacggtgaaattcgtaaacgtccgttgatcgaaaccaacggtgaaaccggtgaaatcgtttgggataaaggtagggattttgcgaccgttcgtaaggtcctgagcatgccgcaggttaacatcgtgaaaaagaccgaagtgcagaccggcggtttctccaaggagagtatcttaccgaagcgcaacagtgacaagctgatcgcgcgtaaaaaagactgggatccgaaaaaatacggcggctttgactccccgaccgtcgcgtacagcgtactggtcgtggctaaagtggagaaaggcaaatcgaaaaagctgaaatccgttaaagagttgctgggtattacgattatggaacgttcgagcttcgagaaaaatccgatcgacttcctggaggccaaaggctacaaggaagttaaaaaagacctgatcattaaactgccgaagtacagcctgttcgagctcgagaacggtcgcaaacgcatgctggcgagcgcgggtgagctccaaaaaggcaacgaattggctctgccgagcaagtacgtgaacttcctgtatctggcctcccattatgaaaaactgaagggtagcccggaagataacgaacaaaagcagctgttcgtggaacaacataaacactatctggacgagattattgagcaaatcagcgagttcagcaaacgcgtgattctggcggatgctaatctggacaaggttctttcagcctataataaacaccgtgataagccgatccgtgaacaggcagaaaatattattcacctgttcaccctgacgaacctgggtgcgccagcagcgtttaaatacttcgacacaacgatcgaccggaagcgttataccagcaccaaagaggttcttgacgccaccttgattcaccaaagcatcacgggtctgtatgaaacccgtattgacttgagccagttgggcggagattcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 5 - DNA, 3363 bp, synthetic construct
  source 1..3363: mol_type = other DNA
atgggaagtcatcaccaccaccaccattcaggtgagaacctgtacttccagagcatggctccgaaaaagaaacgtaaggtgggtatccacggcgttccggcgatggaagcatcaccagctagtggacccaggcacctgatggatccgcacatcttcacctccaattttaataacggcatcggccgtcataagacgtacctgtgctatgaggttgaacgtttggacaacggtactagcgtcaagatggaccagcatcgtggtttccttcacaaccaagcgaaaaatctgctgtgcggtttttatggtcgtcacgccgaactgagattcttggatctggttccgtctctgcaactggacccggcgcagatttatcgtgtgacctggttcatcagctggtcgccgtgctttagctggggttgcgccggtgaagtgcgcgcatttctccaggagaacacccatgttcgtttgcgcatcttcgcggcacgtatcttcgattacgacccgctgtacaaggaggcgctgcaaatgttgcgtgatgctggcgctcaagttagcattatgacctacgacgaatttaaacactgttgggataccttcgtggatcatcagggctgtccgtttcagccatgggacggcctggacgagcacagccaggcgttatccggtcgcctgcgcgcgattctgcaaaaccagggaaactctggaagtgagacacccgggacttcagaatccgcgaccccggagtctggaatgccaaaacccgctgtagagtcagaattttcaaaagttttgaaaaagcactttccgggagaacgtttccgctcgagctatatgaaaagaggcggtaagatcctggctgcccaaggcgaagaggcagttgttgcgtatctacagggcaagtccgaggaggagcctccgaacttccagccaccggcgaagtgccacgtggtgaccaaatcccgtgacttcgcggaatggccgatcatgaaggcgtcggaagcgattcaacgttatatctatgcactgtcgaccaccgaacgcgcggcctgtaaaccgggcaaaagtagtgagagccatgcagcgtggttcgccgctaccggtgtcagcaaccacggctacagccacgttcagggtctgaacctgatttttgatcataccttgggccgttacgatggtgttctgaaaaaggtccaactgcgcaacgaaaaggcgagagcgcgtttagagagcattaacgcgtctagagcagatgaaggtttaccggagattaaagccgaggaggaagaagtggctacgaacgaaaccggccacctgttgcaaccgccgggtatcaatccgagcttttacgtgtatcagaccattagcccgcaggcataccgtccgcgtgacgagatcgtgctgccgccggagtacgcaggttacgtacgtgacccgaatgctccgattccgctgggtgtggtgcgtaaccgttgcgatatccaaaaagggtgcccgggctacatcccggaatggcagcgtgaagcaggtaccgcgatcagcccgaaaacaggcaaggccgtaactgtgccgggcctgtctccgaagaaaaacaaacgtatgcgtaggtactggcgctctgagaaggagaaagctcaagatgcgctgttggttaccgtacgcattggtaccgattgggttgtcatcgacgtgcgcggtctgttgcgcaacgcccgttggcgcaccatcgctcccaaggacatcagcctgaatgcgctcctcgatttgtttaccggtgatccggtgatcgatgtgcgtcgcaatattgttacctttacgtacaccctcgacgcctgtggtacctacgcccgtaaatggaccttgaaaggcaaacagacgaaagcgaccctggacaaactgacggccactcagacggttgcgttggttgcgattgcgcttggtcagaccaacccgatttccgctggcattagccgtgttacacaagaaaatggtgcactgcaatgcgaaccgctggaccggttcaccctgccggatgacctgttgaaggacatcagcgcatatcgcattgcttgggacagaaacgaagaggaactacgcgctcgcagcgtcgaggccctgccggaggcgcaacaagcggaggtccgtgcgctggacggggtgtccaaagaaaccgcgcgtacccaactgtgcgcagatttcggcctggacccgaaacgtctgccgtgggataaaatgagctccaatactacctttatcagcgaagccttgctgtctaacagcgtgagccgtgatcaggtttttttcactccggctccgaagaagggtgctaagaaaaaagcgcctgttgaagttatgcgtaaagaccgcacttgggcacgtgcgtacaagccgcgtctgagcgtcgaagctcagaaactgaagaacgaagccctgtgggcacttaagcgcacctccccggagtatttgaagctgagccgtcgcaaagaggagctgtgtcgtcgtagcatcaactatgttatcgaaaagacccgtcgccgtacgcaatgtcagattgtgatcccggtgatcgaggatctgaatgttcgcttctttcatggttctggtaagagactgcccggttgggataatttcttcaccgctaaaaaagagaaccgttggttcatccagggcctgcacaaagcgtttagcgatctgcgtacgcaccgttccttctatgtttttgaagttcgtccggagcgcacctctattacctgtccaaagtgcggtcattgcgaggtgggtaatcgtgacggtgaggcgtttcagtgcctgagctgcggcaagacgtgcaacgcagatcttgacgtcgcgacccataatctgacccaagtggcgttgaccggtaagaccatgccgaagcgcgaggagccgcgtgacgctcagggtacagcgccagcgcgcaagactaagaaggcgagcaaatcaaaggcgccaccggcggagcgcgaagaccagaccccggcgcaagagccaagccagaccagttctggaggtagcgggggtagcggtggtagcactaacttaagtgatattatcgagaaagaaaccgggaaacagctggtgatccaagaaagcattctgatgctgcccgaagaagttgaagaagtgattggtaacaagcctgagtcggacatcttagtgcacaccgcgtacgatgaaagcacggatgagaatgtgatgctgctcacctctgatgcccccgaatacaagccgtgggctctggtgatccaggactctaacggagaaaacaaaattaagatgctctctggcggctccatggcgcctaaaaaaaaacgcaaagtggggattcacggggttccggcgtaa

SEQ ID NO: 6 - DNA, 4779 bp, synthetic construct
  source 1..4779: mol_type = other DNA
atgggaagtcatcaccaccaccaccattcaggtgagaacctgtacttccagagcatggctccgaaaaagaaacgtaaggtgggtatccacggcgttccggcgatggaagcatcaccagctagtggacccaggcacctgatggatccgcacatcttcacctccaattttaataacggcatcggccgtcataagacgtacctgtgctatgaggttgaacgtttggacaacggtactagcgtcaagatggaccagcatcgtggtttccttcacaaccaagcgaaaaatctgctgtgcggtttttatggtcgtcacgccgaactgagattcttggatctggttccgtctctgcaactggacccggcgcagatttatcgtgtgacctggttcatcagctggtcgccgtgctttagctggggttgcgccggtgaagtgcgcgcatttctccaggagaacacccatgttcgtttgcgcatcttcgcggcacgtatcttcgattacgacccgctgtacaaggaggcgctgcaaatgttgcgtgatgctggcgctcaagttagcattatgacctacgacgaatttaaacactgttgggataccttcgtggatcatcagggctgtccgtttcagccatgggacggcctggacgagcacagccaggcgttatccggtcgcctgcgcgcgattctgcaaaaccagggaaactctggaagtgagacacccgggacttcagaatccgcgaccccggagtctggaatgtcaaaactagaaaagtttacaaattgttactccctgtcgaaaaccctgagattcaaagcgatcccggtgggtaaaactcaagagaacattgataacaaacgtctgctggttgaggacgaaaagcgcgcggaggattacaaaggcgttaaaaagctgttggaccgttattacctgtcctttattaatgatgttctgcacagcattaaattaaagaacctaaacaattacatctccctcttccgtaaaaagacccgcaccgaaaaggaaaataaagaactggagaaccttgagattaatctgcgtaaagaaatcgctaaagcgtttaaaggcaacgagggttataaatccttatttaaaaaagacatcatcgaaacgattctcccagaattcctggacgacaaagacgagatcgcgctggttaattccttcaacggctttaccaccgcattcaccggcttttttgataaccgcgaaaacatgttctctgaagaggccaaaagcacctcaattgcgtttcgctgcattaacgaaaatctgacgcgttatatctctaacatggatatcttcgagaaggttgacgctatctttgataaacatgaagtccaggagatcaaggagaagatcctgaacagcgactacgacgtggaggatttcttcgagggtgagttcttcaattttgttttgacccaagaaggcatcgatgtctacaatgcgataattggtggttttgttaccgagagcggggagaaaattaagggtctgaacgaatatatcaacttgtacaaccagaaaaccaaacaaaaactgccgaagtttaagccgctgtacaagcaagtcttgtcggatcgtgaatcgctgagcttttacggcgaaggctacaccagcgatgaggaggtgttggaggtgttccgcaatacgttgaataagaactctgaaattttttccagcattaagaagctggaaaaactattcaagaacttcgacgagtatagcagcgcaggtatctttgtgaaaaacggtccggctattagcaccatctcgaaagacattttcggcgaatggaatgttattcgtgataagtggaatgccgaatacgacgacattcatctgaagaaaaaggcggttgtaactgaaaagtacgaggacgatcgtcgtaagtcgttcaaaaagatcggctccttctctctggagcaactgcaagagtacgccgatgccgatttgtcagtcgtggagaagctgaaagagatcatcattcaaaaagttgacgaaatctataaggtctatggtagcagtgaaaaactgtttgacgcggacttcgtgctggagaaaagcctgaagaaaaatgacgccgtggtggcaatcatgaaagatcttctggatagcgtcaagtctttcgaaaactacatcaaagcgtttttcggtgaaggcaaggaaacgaatcgtgacgagagcttctatggtgatttcgtcctagcctatgatatcctgcttaaggtggatcacatctatgatgccatccgcaactatgttacccagaaaccgtactcaaaggacaagttcaagttatacttccagaatccgcagtttatgggtggctgggataaagataaggaaaccgactatcgcgccaccattttacgttacggtagtaagtactatctggcaatcatggataaaaaatatgcgaaatgcctgcaaaagatcgacaaggacgatgttaacggcaattatgagaagatcaactacaaactgctgccgggcccaaataagatgctgccgaaggtgtttttcagcaaaaaatggatggcgtattacaatccgagcgaggacatccaaaagatctacaaaaacggtacgtttaagaagggtgatatgttcaacctcaacgattgtcataaacttattgacttcttcaaggatagcatatctcgttatccgaagtggagcaacgcgtatgactttaactttagcgaaaccgagaaatacaaagatatcgcgggtttctatcgtgaagttgaagaacagggttacaaagtgtccttcgagtccgcgagcaagaaggaggtagacaagctggtcgaggagggtaagttgtacatgtttcagatttacaataaggacttttccgacaagtcccatggtacaccgaatttgcacaccatgtatttcaagcttctgttcgacgagaacaaccacggccagatccgtctgagcggtggtgcagagctgttcatgcgtagagctagcctgaagaaagaagagttggtcgttcatccggctaattcgccgatcgcgaacaaaaatccagataaccctaaaaagactacgaccctgagctatgatgtgtataaagacaaacgctttagcgaagaccagtatgagttacacattccgatcgctatcaacaagtgcccgaagaatattttcaagatcaacaccgaagtgcgtgttcttttgaagcacgatgacaacccgtatgtgattggtattgctcgtggtgaacgtaatctgctgtacattgtggttgtggacggcaaaggaaacatcgtggaacagtacagcctgaacgaaatcattaacaatttcaacggcattcgcattaagacggattaccattctttgctggacaaaaaggagaaagagcgtttcgaggcccgtcagaactggacctccatcgagaacattaaggagctgaaggcgggttacatcagccaggtggtgcacaaaatctgcgaactggtggagaagtacgacgcggtgattgctttggcggatttgaactccggttttaagaacagccgcgttaaagtggaaaagcaggtttatcagaaattcgaaaaaatgctgattgataaattgaattacatggttgacaaaaagtctaacccgtgtgcaaccggcggtgcgctgaagggctaccaaatcaccaataagtttgaaagctttaagagcatgagcacgcaaaatggcttcattttttacattccggcgtggctgaccagcaagatcgatccgtcaaccggctttgttaatttactgaagacgaagtatacctctattgccgactctaaaaaatttattagttccttcgatagaatcatgtatgttccggaagaggacctgttcgaatttgcactggactacaagaactttagccgtaccgatgctgattatataaagaaatggaaactctacagctatggcaaccgcatccgtatctttcgcaacccgaaaaaaaacaacgtgttcgactgggaagaggtatgtctgacttcagcgtacaaggagcttttcaataaatatggtatcaactaccagcagggtgatatccgtgcactgctttgcgagcaaagcgataaagcgttctattcaagcttcatggcgctgatgagcctgatgctgcaaatgcgcaacagcatcaccggtcgtaccgatgttgcatttctgatttctccagtaaaaaacagcgatggtatcttttacgacagccgtaattacgaggcccaagaaaatgcgattttgccgaagaacgctgacgcgaatggtgcatacaacattgcaaggaaagttctgtgggcaatcggccaatttaaaaaagcggaagatgagaagctggacaaagtgaaaattgcgattagcaacaaggagtggctggagtatgcgcagacctccgttaaatcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 7 - DNA, 5184 bp, synthetic construct
  source 1..5184: mol_type = other DNA
atgggaagtcatcaccaccaccaccattcaggtgagaacctgtacttccagagcatggctccgaaaaagaaacgtaaggtgggtatccacggcgttccggcgatggaaatactaaggtatttaatggatcccgacactttcaccttcaatttcaacaacgatccattggtcttgcgccgtcgtcagacctacttgtgctatgaggttgaacgtctggataacggtacgtgggttttaatggatcagcatatgggtttcctgtgtaatgaagcgaagaacctgctgtgcggtttttacggccgtcatgcggaactgcgttttcttgacctggttccgagcctgcaactggacccggctcagatttaccgtgttacctggtttatctcgtggagcccgtgttttagctggggctgcgcgggtgaagtgcgcgcgtttttgcaagagaacacccacgtgcgtctccgcatcttcgccgcacgtatttacgattatgacccgctgtataaagaggccctgcaaatgctgagagatgctggcgcacaagtgagcattatgacctacgacgagttcgagtactgctgggatacgttcgtgtatcgccaaggttgcccgtttcagccgtgggacggcctggaggagcacagccaggcgctctccggtcgtttgcgcgcgatcctgcaatctggaagtgagacacccgggacttcagaatccgcgaccccggagtctggaatggataaaaagtattcaataggactagctattggcaccaacagcgttggttgggcagttatcaccgatgaatataaggtcccgagcaagaagtttaaggtcctgggcaacaccgatcgccacagcataaaaaagaaccttattggtgcgctgctgttcgattctggtgagactgccgaagcgacccgtctgaagagaaccgcgcgtcgtagatacacccggcgcaagaaccgtatctgctatctgcaagaaattttcagcaacgagatggctaaggtcgacgactccttctttcatcgcctggaggaaagctttctggttgaggaagacaaaaagcacgagcgtcatccgattttcggtaacattgttgatgaagttgcctaccacgaaaaataccccacaatttatcatctgcgtaaaaaactggtagactccaccgataaggcggatttacgccttatctatttggcgctggcgcatatgattaagttccgcggtcattttttgatcgagggcgacctgaatccggataacagcgacgttgacaaactgtttatccagctggtgcagacctataaccagttgttcgaagagaacccgattaatgcgtctggcgtggacgcgaaggcgatcctgagcgctcgtctgtccaagtcacgccgcctggagaacttgatcgcgcaactgccgggtgagaagaaaaacggcttgttcggcaatctgattgctctgtccctaggtttgaccccgaatttcaaatctaattttgatctggcggaagatgcgaaattacagttgagcaaggacacttacgatgacgatctggacaacctgctcgctcagatcggcgatcagtatgccgatcttttcctggccgcgaagaacctgagcgatgccatcttgctatcggacatcttacgtgttaacaccgaaatcaccaaagctccgctaagcgcgtccatgattaaacgttacgatgaacaccaccaggatctgacgctgctgaaagcgttggtgcgccagcaactgccggaaaaatacaaggaaattttcttcgaccaaagcaagaacggttatgcaggctatatcgacggtggtgcgtcgcaagaagaattttataaattcatcaaaccgattttggagaagatggatggtaccgaggagctgctggtaaaattgaatcgggaagacctcttgcgaaagcagcgtactttcgataatggtagcatcccgcaccagattcatctgggcgaacttcacgcaatcctacgtcgtcaagaggatttctacccgtttctgaaggataaccgtgaaaagatcgagaagattctgacctttcgtattccgtattatgtgggtccgctggcgagaggcaacagccgttttgcgtggatgacccgcaagtcggaagagactatcacgccgtggaatttcgaggaggtcgtggacaagggtgcatcagcgcaatcttttattgaacgtatgaccaattttgataaaaaccttccaaatgaaaaagtgttgccgaagcacagcttgctgtacgagtatttcaccgtttacaatgaactgacgaaagtcaagtacgttaccgaaggtatgcgtaagccagcctttctgtctggggagcaaaaaaaagccattgtggatctgctgttcaagaccaatcgcaaggttaccgtcaagcaactgaaagaggattacttcaaaaaaatcgaatgttttgactccgtggaaatcagcggcgttgaagaccgctttaatgcatctctgggcacctaccacgatctactcaagatcatcaaggataaggacttcctggacaacgaagaaaatgaggatatcttggaagatattgtgctgactttaacgttgttcgaggatcgtgagatgattgaagaacgcctcaaaacgtacgcacatctgtttgacgataaggtgatgaaacagctaaagcgccgtcgttacaccggttggggtcgtctgtctagaaagctgattaacggcattcgtgacaaacaatctggcaagaccatcctggacttcctcaaatccgacggcttcgcaaatcgtaatttcatgcaactgatccatgatgatagcctgacctttaaagaagatattcagaaggctcaagtgtccggccagggtgactcgctgcatgagcatatcgcgaatctggctggctcccctgcgataaaaaaaggtattctccaaacggtgaaagttgtggatgaactggttaaagtcatgggtcgtcataaaccggaaaacatcgtgattgagatggcacgtgagaaccagaccacccagaaaggtcaaaaaaacagtcgtgagcgtatgaaacgtatcgaagaggggattaaggagttaggcagccagattctgaaggagcacccggttgagaacacccagctgcaaaacgagaagctctacctgtattacctgcaaaacggccgtgacatgtatgttgaccaggagctggacatcaatcgtctcagcgactacgacgtggatgctatcgtgccgcagagctttctaaaagatgattccatcgacaataaagtgctcacccgtagcgacaagaaccgtggtaaaagcgacaacgtgccgagcgaggaagtagttaagaaaatgaaaaactactggcgtcagctattgaacgccaagcttatcacccagcgtaagtttgacaacttgacgaaggcagagcgtggcggcctgagcgagctagacaaggcgggttttattaaacgccagctggtggaaactcgccagattaccaaacacgttgcgcaaatcttggactcacgcatgaatacgaagtacgacgaaaacgataaattgatccgcgaggtgaaggtcataaccctgaaatctaaactggttagcgatttccggaaggactttcaattttataaagtccgggagattaacaactatcaccatgcacacgacgcttacttaaatgcggttgttggcacggccctgatcaagaagtacccgaaactggaaagcgagtttgtttatggtgactacaaggtgtatgacgttcgcaagatgattgcaaaatccgaacaagagattggtaaggcgacggctaagtattttttctacagcaatatcatgaacttcttcaagactgaaatcaccctggcgaacggtgaaattcgtaaacgtccgttgatcgaaaccaacggtgaaaccggtgaaatcgtttgggataaaggtagggattttgcgaccgttcgtaaggtcctgagcatgccgcaggttaacatcgtgaaaaagaccgaagtgcagaccggcggtttctccaaggagagtatcttaccgaagcgcaacagtgacaagctgatcgcgcgtaaaaaagactgggatccgaaaaaatacggcggctttgactccccgaccgtcgcgtacagcgtactggtcgtggctaaagtggagaaaggcaaatcgaaaaagctgaaatccgttaaagagttgctgggtattacgattatggaacgttcgagcttcgagaaaaatccgatcgacttcctggaggccaaaggctacaaggaagttaaaaaagacctgatcattaaactgccgaagtacagcctgttcgagctcgagaacggtcgcaaacgcatgctggcgagcgcgggtgagctccaaaaaggcaacgaattggctctgccgagcaagtacgtgaacttcctgtatctggcctcccattatgaaaaactgaagggtagcccggaagataacgaacaaaagcagctgttcgtggaacaacataaacactatctggacgagattattgagcaaatcagcgagttcagcaaacgcgtgattctggcggatgctaatctggacaaggttctttcagcctataataaacaccgtgataagccgatccgtgaacaggcagaaaatattattcacctgttcaccctgacgaacctgggtgcgccagcagcgtttaaatacttcgacacaacgatcgaccggaagcgttataccagcaccaaagaggttcttgacgccaccttgattcaccaaagcatcacgggtctgtatgaaacccgtattgacttgagccagttgggcggagattcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 8 - DNA, 3345 bp, synthetic construct
  source 1..3345: mol_type = other DNA
atgggaagtcatcaccaccaccaccattcaggtgagaacctgtacttccagagcatggctccgaaaaagaaacgtaaggtgggtatccacggcgttccggcgatggaaatactaaggtatttaatggatcccgacactttcaccttcaatttcaacaacgatccattggtcttgcgccgtcgtcagacctacttgtgctatgaggttgaacgtctggataacggtacgtgggttttaatggatcagcatatgggtttcctgtgtaatgaagcgaagaacctgctgtgcggtttttacggccgtcatgcggaactgcgttttcttgacctggttccgagcctgcaactggacccggctcagatttaccgtgttacctggtttatctcgtggagcccgtgttttagctggggctgcgcgggtgaagtgcgcgcgtttttgcaagagaacacccacgtgcgtctccgcatcttcgccgcacgtatttacgattatgacccgctgtataaagaggccctgcaaatgctgagagatgctggcgcacaagtgagcattatgacctacgacgagttcgagtactgctgggatacgttcgtgtatcgccaaggttgcccgtttcagccgtgggacggcctggaggagcacagccaggcgctctccggtcgtttgcgcgcgatcctgcaatctggaagtgagacacccgggacttcagaatccgcgaccccggagtctggaatgccaaaacccgctgtagagtcagaattttcaaaagttttgaaaaagcactttccgggagaacgtttccgctcgagctatatgaaaagaggcggtaagatcctggctgcccaaggcgaagaggcagttgttgcgtatctacagggcaagtccgaggaggagcctccgaacttccagccaccggcgaagtgccacgtggtgaccaaatcccgtgacttcgcggaatggccgatcatgaaggcgtcggaagcgattcaacgttatatctatgcactgtcgaccaccgaacgcgcggcctgtaaaccgggcaaaagtagtgagagccatgcagcgtggttcgccgctaccggtgtcagcaaccacggctacagccacgttcagggtctgaacctgatttttgatcataccttgggccgttacgatggtgttctgaaaaaggtccaactgcgcaacgaaaaggcgagagcgcgtttagagagcattaacgcgtctagagcagatgaaggtttaccggagattaaagccgaggaggaagaagtggctacgaacgaaaccggccacctgttgcaaccgccgggtatcaatccgagcttttacgtgtatcagaccattagcccgcaggcataccgtccgcgtgacgagatcgtgctgccgccggagtacgcaggttacgtacgtgacccgaatgctccgattccgctgggtgtggtgcgtaaccgttgcgatatccaaaaagggtgcccgggctacatcccggaatggcagcgtgaagcaggtaccgcgatcagcccgaaaacaggcaaggccgtaactgtgccgggcctgtctccgaagaaaaacaaacgtatgcgtaggtactggcgctctgagaaggagaaagctcaagatgcgctgttggttaccgtacgcattggtaccgattgggttgtcatcgacgtgcgcggtctgttgcgcaacgcccgttggcgcaccatcgctcccaaggacatcagcctgaatgcgctcctcgatttgtttaccggtgatccggtgatcgatgtgcgtcgcaatattgttacctttacgtacaccctcgacgcctgtggtacctacgcccgtaaatggaccttgaaaggcaaacagacgaaagcgaccctggacaaactgacggccactcagacggttgcgttggttgcgattgcgcttggtcagaccaacccgatttccgctggcattagccgtgttacacaagaaaatggtgcactgcaatgcgaaccgctggaccggttcaccctgccggatgacctgttgaaggacatcagcgcatatcgcattgcttgggacagaaacgaagaggaactacgcgctcgcagcgtcgaggccctgccggaggcgcaacaagcggaggtccgtgcgctggacggggtgtccaaagaaaccgcgcgtacccaactgtgcgcagatttcggcctggacccgaaacgtctgccgtgggataaaatgagctccaatactacctttatcagcgaagccttgctgtctaacagcgtgagccgtgatcaggtttttttcactccggctccgaagaagggtgctaagaaaaaagcgcctgttgaagttatgcgtaaagaccgcacttgggcacgtgcgtacaagccgcgtctgagcgtcgaagctcagaaactgaagaacgaagccctgtgggcacttaagcgcacctccccggagtatttgaagctgagccgtcgcaaagaggagctgtgtcgtcgtagcatcaactatgttatcgaaaagacccgtcgccgtacgcaatgtcagattgtgatcccggtgatcgaggatctgaatgttcgcttctttcatggttctggtaagagactgcccggttgggataatttcttcaccgctaaaaaagagaaccgttggttcatccagggcctgcacaaagcgtttagcgatctgcgtacgcaccgttccttctatgtttttgaagttcgtccggagcgcacctctattacctgtccaaagtgcggtcattgcgaggtgggtaatcgtgacggtgaggcgtttcagtgcctgagctgcggcaagacgtgcaacgcagatcttgacgtcgcgacccataatctgacccaagtggcgttgaccggtaagaccatgccgaagcgcgaggagccgcgtgacgctcagggtacagcgccagcgcgcaagactaagaaggcgagcaaatcaaaggcgccaccggcggagcgcgaagaccagaccccggcgcaagagccaagccagaccagttctggaggtagcgggggtagcggtggtagcactaacttaagtgatattatcgagaaagaaaccgggaaacagctggtgatccaagaaagcattctgatgctgcccgaagaagttgaagaagtgattggtaacaagcctgagtcggacatcttagtgcacaccgcgtacgatgaaagcacggatgagaatgtgatgctgctcacctctgatgcccccgaatacaagccgtgggctctggtgatccaggactctaacggagaaaacaaaattaagatgctctctggcggctccatggcgcctaaaaaaaaacgcaaagtggggattcacggggttccggcgtaa

SEQ ID NO: 9 - DNA, 4761 bp, synthetic construct
  source 1..4761: mol_type = other DNA
atgggaagtcatcaccaccaccaccattcaggtgagaacctgtacttccagagcatggctccgaaaaagaaacgtaaggtgggtatccacggcgttccggcgatggaaatactaaggtatttaatggatcccgacactttcaccttcaatttcaacaacgatccattggtcttgcgccgtcgtcagacctacttgtgctatgaggttgaacgtctggataacggtacgtgggttttaatggatcagcatatgggtttcctgtgtaatgaagcgaagaacctgctgtgcggtttttacggccgtcatgcggaactgcgttttcttgacctggttccgagcctgcaactggacccggctcagatttaccgtgttacctggtttatctcgtggagcccgtgttttagctggggctgcgcgggtgaagtgcgcgcgtttttgcaagagaacacccacgtgcgtctccgcatcttcgccgcacgtatttacgattatgacccgctgtataaagaggccctgcaaatgctgagagatgctggcgcacaagtgagcattatgacctacgacgagttcgagtactgctgggatacgttcgtgtatcgccaaggttgcccgtttcagccgtgggacggcctggaggagcacagccaggcgctctccggtcgtttgcgcgcgatcctgcaatctggaagtgagacacccgggacttcagaatccgcgaccccggagtctggaatgtcaaaactagaaaagtttacaaattgttactccctgtcgaaaaccctgagattcaaagcgatcccggtgggtaaaactcaagagaacattgataacaaacgtctgctggttgaggacgaaaagcgcgcggaggattacaaaggcgttaaaaagctgttggaccgttattacctgtcctttattaatgatgttctgcacagcattaaattaaagaacctaaacaattacatctccctcttccgtaaaaagacccgcaccgaaaaggaaaataaagaactggagaaccttgagattaatctgcgtaaagaaatcgctaaagcgtttaaaggcaacgagggttataaatccttatttaaaaaagacatcatcgaaacgattctcccagaattcctggacgacaaagacgagatcgcgctggttaattccttcaacggctttaccaccgcattcaccggcttttttgataaccgcgaaaacatgttctctgaagaggccaaaagcacctcaattgcgtttcgctgcattaacgaaaatctgacgcgttatatctctaacatggatatcttcgagaaggttgacgctatctttgataaacatgaagtccaggagatcaaggagaagatcctgaacagcgactacgacgtggaggatttcttcgagggtgagttcttcaattttgttttgacccaagaaggcatcgatgtctacaatgcgataattggtggttttgttaccgagagcggggagaaaattaagggtctgaacgaatatatcaacttgtacaaccagaaaaccaaacaaaaactgccgaagtttaagccgctgtacaagcaagtcttgtcggatcgtgaatcgctgagcttttacggcgaaggctacaccagcgatgaggaggtgttggaggtgttccgcaatacgttgaataagaactctgaaattttttccagcattaagaagctggaaaaactattcaagaacttcgacgagtatagcagcgcaggtatctttgtgaaaaacggtccggctattagcaccatctcgaaagacattttcggcgaatggaatgttattcgtgataagtggaatgccgaatacgacgacattcatctgaagaaaaaggcggttgtaactgaaaagtacgaggacgatcgtcgtaagtcgttcaaaaagatcggctccttctctctggagcaactgcaagagtacgccgatgccgatttgtcagtcgtggagaagctgaaagagatcatcattcaaaaagttgacgaaatctataaggtctatggtagcagtgaaaaactgtttgacgcggacttcgtgctggagaaaagcctgaagaaaaatgacgccgtggtggcaatcatgaaagatcttctggatagcgtcaagtctttcgaaaactacatcaaagcgtttttcggtgaaggcaaggaaacgaatcgtgacgagagcttctatggtgatttcgtcctagcctatgatatcctgcttaaggtggatcacatctatgatgccatccgcaactatgttacccagaaaccgtactcaaaggacaagttcaagttatacttccagaatccgcagtttatgggtggctgggataaagataaggaaaccgactatcgcgccaccattttacgttacggtagtaagtactatctggcaatcatggataaaaaatatgcgaaatgcctgcaaaagatcgacaaggacgatgttaacggcaattatgagaagatcaactacaaactgctgccgggcccaaataagatgctgccgaaggtgtttttcagcaaaaaatggatggcgtattacaatccgagcgaggacatccaaaagatctacaaaaacggtacgtttaagaagggtgatatgttcaacctcaacgattgtcataaacttattgacttcttcaaggatagcatatctcgttatccgaagtggagcaacgcgtatgactttaactttagcgaaaccgagaaatacaaagatatcgcgggtttctatcgtgaagttgaagaacagggttacaaagtgtccttcgagtccgcgagcaagaaggaggtagacaagctggtcgaggagggtaagttgtacatgtttcagatttacaataaggacttttccgacaagtcccatggtacaccgaatttgcacaccatgtatttcaagcttctgttcgacgagaacaaccacggccagatccgtctgagcggtggtgcagagctgttcatgcgtagagctagcctgaagaaagaagagttggtcgttcatccggctaattcgccgatcgcgaacaaaaatccagataaccctaaaaagactacgaccctgagctatgatgtgtataaagacaaacgctttagcgaagaccagtatgagttacacattccgatcgctatcaacaagtgcccgaagaatattttcaagatcaacaccgaagtgcgtgttcttttgaagcacgatgacaacccgtatgtgattggtattgctcgtggtgaacgtaatctgctgtacattgtggttgtggacggcaaaggaaacatcgtggaacagtacagcctgaacgaaatcattaacaatttcaacggcattcgcattaagacggattaccattctttgctggacaaaaaggagaaagagcgtttcgaggcccgtcagaactggacctccatcgagaacattaaggagctgaaggcgggttacatcagccaggtggtgcacaaaatctgcgaactggtggagaagtacgacgcggtgattgctttggcggatttgaactccggttttaagaacagccgcgttaaagtggaaaagcaggtttatcagaaattcgaaaaaatgctgattgataaattgaattacatggttgacaaaaagtctaacccgtgtgcaaccggcggtgcgctgaagggctaccaaatcaccaataagtttgaaagctttaagagcatgagcacgcaaaatggcttcattttttacattccggcgtggctgaccagcaagatcgatccgtcaaccggctttgttaatttactgaagacgaagtatacctctattgccgactctaaaaaatttattagttccttcgatagaatcatgtatgttccggaagaggacctgttcgaatttgcactggactacaagaactttagccgtaccgatgctgattatataaagaaatggaaactctacagctatggcaaccgcatccgtatctttcgcaacccgaaaaaaaacaacgtgttcgactgggaagaggtatgtctgacttcagcgtacaaggagcttttcaataaatatggtatcaactaccagcagggtgatatccgtgcactgctttgcgagcaaagcgataaagcgttctattcaagcttcatggcgctgatgagcctgatgctgcaaatgcgcaacagcatcaccggtcgtaccgatgttgcatttctgatttctccagtaaaaaacagcgatggtatcttttacgacagccgtaattacgaggcccaagaaaatgcgattttgccgaagaacgctgacgcgaatggtgcatacaacattgcaaggaaagttctgtgggcaatcggccaatttaaaaaagcggaagatgagaagctggacaaagtgaaaattgcgattagcaacaaggagtggctggagtatgcgcagacctccgttaaatcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 10 - DNA, 5199 bp, synthetic construct
  source 1..5199: mol_type = other DNA
atgggaagtcatcaccaccaccaccattcaggtgagaacctgtacttccagagcatggctccgaaaaagaaacgtaaggtgggtatccacggcgttccggcgatggattcactattaatgaataggagaaaattcctgtatcagtttaagaacgtccgttgggctaaaggtcgtcgtgagacgtatttatgctacgtggtgaaacgtcgcgacagcgcaacctccttcagcctggacttcggctatctgcgtaacaaaaacggctgccacgtggagctgctgtttttgcgctacattagcgactgggatttggacccaggtcgctgctatcgtgttacctggtttacgagctggtcgccgtgttacgactgtgcgcgtcatgtcgcggattttctgcgcggtaatccgaatctgtccctgcgtatttttaccgcacgtctctacttctgcgaagatcgtaaggcggaaccggaaggcttgcgtcgcctgcatagagcgggtgttcagatcgctatcatgaccttcaaagattatttctactgctggaataccttcgtggagaaccacgagcgtactttcaaggcctgggaaggccttcacgaaaacagcgttcgcttgtctcgtcaactgcgccgcatcctgctgccgctgtacgaggttgatgatctcagagacgcgtttcgtaccctgggtttgtctggaagtgagacacccgggacttcagaatccgcgaccccggagtctggaatggataaaaagtattcaataggactagctattggcaccaacagcgttggttgggcagttatcaccgatgaatataaggtcccgagcaagaagtttaaggtcctgggcaacaccgatcgccacagcataaaaaagaaccttattggtgcgctgctgttcgattctggtgagactgccgaagcgacccgtctgaagagaaccgcgcgtcgtagatacacccggcgcaagaaccgtatctgctatctgcaagaaattttcagcaacgagatggctaaggtcgacgactccttctttcatcgcctggaggaaagctttctggttgaggaagacaaaaagcacgagcgtcatccgattttcggtaacattgttgatgaagttgcctaccacgaaaaataccccacaatttatcatctgcgtaaaaaactggtagactccaccgataaggcggatttacgccttatctatttggcgctggcgcatatgattaagttccgcggtcattttttgatcgagggcgacctgaatccggataacagcgacgttgacaaactgtttatccagctggtgcagacctataaccagttgttcgaagagaacccgattaatgcgtctggcgtggacgcgaaggcgatcctgagcgctcgtctgtccaagtcacgccgcctggagaacttgatcgcgcaactgccgggtgagaagaaaaacggcttgttcggcaatctgattgctctgtccctaggtttgaccccgaatttcaaatctaattttgatctggcggaagatgcgaaattacagttgagcaaggacacttacgatgacgatctggacaacctgctcgctcagatcggcgatcagtatgccgatcttttcctggccgcgaagaacctgagcgatgccatcttgctatcggacatcttacgtgttaacaccgaaatcaccaaagctccgctaagcgcgtccatgattaaacgttacgatgaacaccaccaggatctgacgctgctgaaagcgttggtgcgccagcaactgccggaaaaatacaaggaaattttcttcgaccaaagcaagaacggttatgcaggctatatcgacggtggtgcgtcgcaagaagaattttataaattcatcaaaccgattttggagaagatggatggtaccgaggagctgctggtaaaattgaatcgggaagacctcttgcgaaagcagcgtactttcgataatggtagcatcccgcaccagattcatctgggcgaacttcacgcaatcctacgtcgtcaagaggatttctacccgtttctgaaggataaccgtgaaaagatcgagaagattctgacctttcgtattccgtattatgtgggtccgctggcgagaggcaacagccgttttgcgtggatgacccgcaagtcggaagagactatcacgccgtggaatttcgaggaggtcgtggacaagggtgcatcagcgcaatcttttattgaacgtatgaccaattttgataaaaaccttccaaatgaaaaagtgttgccgaagcacagcttgctgtacgagtatttcaccgtttacaatgaactgacgaaagtcaagtacgttaccgaaggtatgcgtaagccagcctttctgtctggggagcaaaaaaaagccattgtggatctgctgttcaagaccaatcgcaaggttaccgtcaagcaactgaaagaggattacttcaaaaaaatcgaatgttttgactccgtggaaatcagcggcgttgaagaccgctttaatgcatctctgggcacctaccacgatctactcaagatcatcaaggataaggacttcctggacaacgaagaaaatgaggatatcttggaagatattgtgctgactttaacgttgttcgaggatcgtgagatgattgaagaacgcctcaaaacgtacgcacatctgtttgacgataaggtgatgaaacagctaaagcgccgtcgttacaccggttggggtcgtctgtctagaaagctgattaacggcattcgtgacaaacaatctggcaagaccatcctggacttcctcaaatccgacggcttcgcaaatcgtaatttcatgcaactgatccatgatgatagcctgacctttaaagaagatattcagaaggctcaagtgtccggccagggtgactcgctgcatgagcatatcgcgaatctggctggctcccctgcgataaaaaaaggtattctccaaacggtgaaagttgtggatgaactggttaaagtcatgggtcgtcataaaccggaaaacatcgtgattgagatggcacgtgagaaccagaccacccagaaaggtcaaaaaaacagtcgtgagcgtatgaaacgtatcgaagaggggattaaggagttaggcagccagattctgaaggagcacccggttgagaacacccagctgcaaaacgagaagctctacctgtattacctgcaaaacggccgtgacatgtatgttgaccaggagctggacatcaatcgtctcagcgactacgacgtggatgctatcgtgccgcagagctttctaaaagatgattccatcgacaataaagtgctcacccgtagcgacaagaaccgtggtaaaagcgacaacgtgccgagcgaggaagtagttaagaaaatgaaaaactactggcgtcagctattgaacgccaagcttatcacccagcgtaagtttgacaacttgacgaaggcagagcgtggcggcctgagcgagctagacaaggcgggttttattaaacgccagctggtggaaactcgccagattaccaaacacgttgcgcaaatcttggactcacgcatgaatacgaagtacgacgaaaacgataaattgatccgcgaggtgaaggtcataaccctgaaatctaaactggttagcgatttccggaaggactttcaattttataaagtccgggagattaacaactatcaccatgcacacgacgcttacttaaatgcggttgttggcacggccctgatcaagaagtacccgaaactggaaagcgagtttgtttatggtgactacaaggtgtatgacgttcgcaagatgattgcaaaatccgaacaagagattggtaaggcgacggctaagtattttttctacagcaatatcatgaacttcttcaagactgaaatcaccctggcgaacggtgaaattcgtaaacgtccgttgatcgaaaccaacggtgaaaccggtgaaatcgtttgggataaaggtagggattttgcgaccgttcgtaaggtcctgagcatgccgcaggttaacatcgtgaaaaagaccgaagtgcagaccggcggtttctccaaggagagtatcttaccgaagcgcaacagtgacaagctgatcgcgcgtaaaaaagactgggatccgaaaaaatacggcggctttgactccccgaccgtcgcgtacagcgtactggtcgtggctaaagtggagaaaggcaaatcgaaaaagctgaaatccgttaaagagttgctgggtattacgattatggaacgttcgagcttcgagaaaaatccgatcgacttcctggaggccaaaggctacaaggaagttaaaaaagacctgatcattaaactgccgaagtacagcctgttcgagctcgagaacggtcgcaaacgcatgctggcgagcgcgggtgagctccaaaaaggcaacgaattggctctgccgagcaagtacgtgaacttcctgtatctggcctcccattatgaaaaactgaagggtagcccggaagataacgaacaaaagcagctgttcgtggaacaacataaacactatctggacgagattattgagcaaatcagcgagttcagcaaacgcgtgattctggcggatgctaatctggacaaggttctttcagcctataataaacaccgtgataagccgatccgtgaacaggcagaaaatattattcacctgttcaccctgacgaacctgggtgcgccagcagcgtttaaatacttcgacacaacgatcgaccggaagcgttataccagcaccaaagaggttcttgacgccaccttgattcaccaaagcatcacgggtctgtatgaaacccgtattgacttgagccagttgggcggagattcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 11 - DNA, 3360 bp, synthetic construct
  source 1..3360: mol_type = other DNA
atgggaagtcatcaccaccaccaccattcaggtgagaacctgtacttccagagcatggctccgaaaaagaaacgtaaggtgggtatccacggcgttccggcgatggattcactattaatgaataggagaaaattcctgtatcagtttaagaacgtccgttgggctaaaggtcgtcgtgagacgtatttatgctacgtggtgaaacgtcgcgacagcgcaacctccttcagcctggacttcggctatctgcgtaacaaaaacggctgccacgtggagctgctgtttttgcgctacattagcgactgggatttggacccaggtcgctgctatcgtgttacctggtttacgagctggtcgccgtgttacgactgtgcgcgtcatgtcgcggattttctgcgcggtaatccgaatctgtccctgcgtatttttaccgcacgtctctacttctgcgaagatcgtaaggcggaaccggaaggcttgcgtcgcctgcatagagcgggtgttcagatcgctatcatgaccttcaaagattatttctactgctggaataccttcgtggagaaccacgagcgtactttcaaggcctgggaaggccttcacgaaaacagcgttcgcttgtctcgtcaactgcgccgcatcctgctgccgctgtacgaggttgatgatctcagagacgcgtttcgtaccctgggtttgtctggaagtgagacacccgggacttcagaatccgcgaccccggagtctggaatgccaaaacccgctgtagagtcagaattttcaaaagttttgaaaaagcactttccgggagaacgtttccgctcgagctatatgaaaagaggcggtaagatcctggctgcccaaggcgaagaggcagttgttgcgtatctacagggcaagtccgaggaggagcctccgaacttccagccaccggcgaagtgccacgtggtgaccaaatcccgtgacttcgcggaatggccgatcatgaaggcgtcggaagcgattcaacgttatatctatgcactgtcgaccaccgaacgcgcggcctgtaaaccgggcaaaagtagtgagagccatgcagcgtggttcgccgctaccggtgtcagcaaccacggctacagccacgttcagggtctgaacctgatttttgatcataccttgggccgttacgatggtgttctgaaaaaggtccaactgcgcaacgaaaaggcgagagcgcgtttagagagcattaacgcgtctagagcagatgaaggtttaccggagattaaagccgaggaggaagaagtggctacgaacgaaaccggccacctgttgcaaccgccgggtatcaatccgagcttttacgtgtatcagaccattagcccgcaggcataccgtccgcgtgacgagatcgtgctgccgccggagtacgcaggttacgtacgtgacccgaatgctccgattccgctgggtgtggtgcgtaaccgttgcgatatccaaaaagggtgcccgggctacatcccggaatggcagcgtgaagcaggtaccgcgatcagcccgaaaacaggcaaggccgtaactgtgccgggcctgtctccgaagaaaaacaaacgtatgcgtaggtactggcgctctgagaaggagaaagctcaagatgcgctgttggttaccgtacgcattggtaccgattgggttgtcatcgacgtgcgcggtctgttgcgcaacgcccgttggcgcaccatcgctcccaaggacatcagcctgaatgcgctcctcgatttgtttaccggtgatccggtgatcgatgtgcgtcgcaatattgttacctttacgtacaccctcgacgcctgtggtacctacgcccgtaaatggaccttgaaaggcaaacagacgaaagcgaccctggacaaactgacggccactcagacggttgcgttggttgcgattgcgcttggtcagaccaacccgatttccgctggcattagccgtgttacacaagaaaatggtgcactgcaatgcgaaccgctggaccggttcaccctgccggatgacctgttgaaggacatcagcgcatatcgcattgcttgggacagaaacgaagaggaactacgcgctcgcagcgtcgaggccctgccggaggcgcaacaagcggaggtccgtgcgctggacggggtgtccaaagaaaccgcgcgtacccaactgtgcgcagatttcggcctggacccgaaacgtctgccgtgggataaaatgagctccaatactacctttatcagcgaagccttgctgtctaacagcgtgagccgtgatcaggtttttttcactccggctccgaagaagggtgctaagaaaaaagcgcctgttgaagttatgcgtaaagaccgcacttgggcacgtgcgtacaagccgcgtctgagcgtcgaagctcagaaactgaagaacgaagccctgtgggcacttaagcgcacctccccggagtatttgaagctgagccgtcgcaaagaggagctgtgtcgtcgtagcatcaactatgttatcgaaaagacccgtcgccgtacgcaatgtcagattgtgatcccggtgatcgaggatctgaatgttcgcttctttcatggttctggtaagagactgcccggttgggataatttcttcaccgctaaaaaagagaaccgttggttcatccagggcctgcacaaagcgtttagcgatctgcgtacgcaccgttccttctatgtttttgaagttcgtccggagcgcacctctattacctgtccaaagtgcggtcattgcgaggtgggtaatcgtgacggtgaggcgtttcagtgcctgagctgcggcaagacgtgcaacgcagatcttgacgtcgcgacccataatctgacccaagtggcgttgaccggtaagaccatgccgaagcgcgaggagccgcgtgacgctcagggtacagcgccagcgcgcaagactaagaaggcgagcaaatcaaaggcgccaccggcggagcgcgaagaccagaccccggcgcaagagccaagccagaccagttctggaggtagcgggggtagcggtggtagcactaacttaagtgatattatcgagaaagaaaccgggaaacagctggtgatccaagaaagcattctgatgctgcccgaagaagttgaagaagtgattggtaacaagcctgagtcggacatcttagtgcacaccgcgtacgatgaaagcacggatgagaatgtgatgctgctcacctctgatgcccccgaatacaagccgtgggctctggtgatccaggactctaacggagaaaacaaaattaagatgctctctggcggctccatggcgcctaaaaaaaaacgcaaagtggggattcacggggttccggcgtaa

SEQ ID NO: 12 - DNA, 4776 bp, synthetic construct
  source 1..4776: mol_type = other DNA
atgggaagtcatcaccaccaccaccattcaggtgagaacctgtacttccagagcatggctccgaaaaagaaacgtaaggtgggtatccacggcgttccggcgatggattcactattaatgaataggagaaaattcctgtatcagtttaagaacgtccgttgggctaaaggtcgtcgtgagacgtatttatgctacgtggtgaaacgtcgcgacagcgcaacctccttcagcctggacttcggctatctgcgtaacaaaaacggctgccacgtggagctgctgtttttgcgctacattagcgactgggatttggacccaggtcgctgctatcgtgttacctggtttacgagctggtcgccgtgttacgactgtgcgcgtcatgtcgcggattttctgcgcggtaatccgaatctgtccctgcgtatttttaccgcacgtctctacttctgcgaagatcgtaaggcggaaccggaaggcttgcgtcgcctgcatagagcgggtgttcagatcgctatcatgaccttcaaagattatttctactgctggaataccttcgtggagaaccacgagcgtactttcaaggcctgggaaggccttcacgaaaacagcgttcgcttgtctcgtcaactgcgccgcatcctgctgccgctgtacgaggttgatgatctcagagacgcgtttcgtaccctgggtttgtctggaagtgagacacccgggacttcagaatccgcgaccccggagtctggaatgtcaaaactagaaaagtttacaaattgttactccctgtcgaaaaccctgagattcaaagcgatcccggtgggtaaaactcaagagaacattgataacaaacgtctgctggttgaggacgaaaagcgcgcggaggattacaaaggcgttaaaaagctgttggaccgttattacctgtcctttattaatgatgttctgcacagcattaaattaaagaacctaaacaattacatctccctcttccgtaaaaagacccgcaccgaaaaggaaaataaagaactggagaaccttgagattaatctgcgtaaagaaatcgctaaagcgtttaaaggcaacgagggttataaatccttatttaaaaaagacatcatcgaaacgattctcccagaattcctggacgacaaagacgagatcgcgctggttaattccttcaacggctttaccaccgcattcaccggcttttttgataaccgcgaaaacatgttctctgaagaggccaaaagcacctcaattgcgtttcgctgcattaacgaaaatctgacgcgttatatctctaacatggatatcttcgagaaggttgacgctatctttgataaacatgaagtccaggagatcaaggagaagatcctgaacagcgactacgacgtggaggatttcttcgagggtgagttcttcaattttgttttgacccaagaaggcatcgatgtctacaatgcgataattggtggttttgttaccgagagcggggagaaaattaagggtctgaacgaatatatcaacttgtacaaccagaaaaccaaacaaaaactgccgaagtttaagccgctgtacaagcaagtcttgtcggatcgtgaatcgctgagcttttacggcgaaggctacaccagcgatgaggaggtgttggaggtgttccgcaatacgttgaataagaactctgaaattttttccagcattaagaagctggaaaaactattcaagaacttcgacgagtatagcagcgcaggtatctttgtgaaaaacggtccggctattagcaccatctcgaaagacattttcggcgaatggaatgttattcgtgataagtggaatgccgaatacgacgacattcatctgaagaaaaaggcggttgtaactgaaaagtacgaggacgatcgtcgtaagtcgttcaaaaagatcggctccttctctctggagcaactgcaagagtacgccgatgccgatttgtcagtcgtggagaagctgaaagagatcatcattcaaaaagttgacgaaatctataaggtctatggtagcagtgaaaaactgtttgacgcggacttcgtgctggagaaaagcctgaagaaaaatgacgccgtggtggcaatcatgaaagatcttctggatagcgtcaagtctttcgaaaactacatcaaagcgtttttcggtgaaggcaaggaaacgaatcgtgacgagagcttctatggtgatttcgtcctagcctatgatatcctgcttaaggtggatcacatctatgatgccatccgcaactatgttacccagaaaccgtactcaaaggacaagttcaagttatacttccagaatccgcagtttatgggtggctgggataaagataaggaaaccgactatcgcgccaccattttacgttacggtagtaagtactatctggcaatcatggataaaaaatatgcgaaatgcctgcaaaagatcgacaaggacgatgttaacggcaattatgagaagatcaactacaaactgctgccgggcccaaataagatgctgccgaaggtgtttttcagcaaaaaatggatggcgtattacaatccgagcgaggacatccaaaagatctacaaaaacggtacgtttaagaagggtgatatgttcaacctcaacgattgtcataaacttattgacttcttcaaggatagcatatctcgttatccgaagtggagcaacgcgtatgactttaactttagcgaaaccgagaaatacaaagatatcgcgggtttctatcgtgaagttgaagaacagggttacaaagtgtccttcgagtccgcgagcaagaaggaggtagacaagctggtcgaggagggtaagttgtacatgtttcagatttacaataaggacttttccgacaagtcccatggtacaccgaatttgcacaccatgtatttcaagcttctgttcgacgagaacaaccacggccagatccgtctgagcggtggtgcagagctgttcatgcgtagagctagcctgaagaaagaagagttggtcgttcatccggctaattcgccgatcgcgaacaaaaatccagataaccctaaaaagactacgaccctgagctatgatgtgtataaagacaaacgctttagcgaagaccagtatgagttacacattccgatcgctatcaacaagtgcccgaagaatattttcaagatcaacaccgaagtgcgtgttcttttgaagcacgatgacaacccgtatgtgattggtattgctcgtggtgaacgtaatctgctgtacattgtggttgtggacggcaaaggaaacatcgtggaacagtacagcctgaacgaaatcattaacaatttcaacggcattcgcattaagacggattaccattctttgctggacaaaaaggagaaagagcgtttcgaggcccgtcagaactggacctccatcgagaacattaaggagctgaaggcgggttacatcagccaggtggtgcacaaaatctgcgaactggtggagaagtacgacgcggtgattgctttggcggatttgaactccggttttaagaacagccgcgttaaagtggaaaagcaggtttatcagaaattcgaaaaaatgctgattgataaattgaattacatggttgacaaaaagtctaacccgtgtgcaaccggcggtgcgctgaagggctaccaaatcaccaataagtttgaaagctttaagagcatgagcacgcaaaatggcttcattttttacattccggcgtggctgaccagcaagatcgatccgtcaaccggctttgttaatttactgaagacgaagtatacctctattgccgactctaaaaaatttattagttccttcgatagaatcatgtatgttccggaagaggacctgttcgaatttgcactggactacaagaactttagccgtaccgatgctgattatataaagaaatggaaactctacagctatggcaaccgcatccgtatctttcgcaacccgaaaaaaaacaacgtgttcgactgggaagaggtatgtctgacttcagcgtacaaggagcttttcaataaatatggtatcaactaccagcagggtgatatccgtgcactgctttgcgagcaaagcgataaagcgttctattcaagcttcatggcgctgatgagcctgatgctgcaaatgcgcaacagcatcaccggtcgtaccgatgttgcatttctgatttctccagtaaaaaacagcgatggtatcttttacgacagccgtaattacgaggcccaagaaaatgcgattttgccgaagaacgctgacgcgaatggtgcatacaacattgcaaggaaagttctgtgggcaatcggccaatttaaaaaagcggaagatgagaagctggacaaagtgaaaattgcgattagcaacaaggagtggctggagtatgcgcagacctccgttaaatcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 13 - DNA, 5181 bp, synthetic construct
  source 1..5181: mol_type = other DNA
atgggaagtcatcaccaccaccaccattcaggtgagaacctgtacttccagagcatggctccgaaaaagaaacgtaaggtgggtatccacggcgttccggcgatggctgattcaagtgaaaaaatgaggggacaatatatcagccgtgacacctttgagaaaaactacaaaccgattgacggcacgaaagaggcgcatctgctgtgcgaaattaaatggggtaagtacggcaagccgtggctgcattggtgtcaaaaccaacgtatgaatatccatgctgaagactacttcatgaacaacatcttcaaagccaaaaagcatccggtgcactgctacgtgacctggtatttgtcctggtcaccgtgcgcggattgtgcgagcaagatcgttaagttcctggaggagcgtccttatctgaaactgaccatttatgttgcacagctgtattatcacactgaagaggagaaccgtaaaggtctgcgtttgctgcgttcgaagaaggtgattatccgcgtgatggatatctctgattacaactactgctggaaagtctttgttagcaatcagaacggtaatgaagactactggccgctgcaattcgatccgtgggttaaggagaactacagccgcctcttggacattttctgggaaagcaagtgccgttccccaaatccgtggtctggaagtgagacacccgggacttcagaatccgcgaccccggagtctggaatggataaaaagtattcaataggactagctattggcaccaacagcgttggttgggcagttatcaccgatgaatataaggtcccgagcaagaagtttaaggtcctgggcaacaccgatcgccacagcataaaaaagaaccttattggtgcgctgctgttcgattctggtgagactgccgaagcgacccgtctgaagagaaccgcgcgtcgtagatacacccggcgcaagaaccgtatctgctatctgcaagaaattttcagcaacgagatggctaaggtcgacgactccttctttcatcgcctggaggaaagctttctggttgaggaagacaaaaagcacgagcgtcatccgattttcggtaacattgttgatgaagttgcctaccacgaaaaataccccacaatttatcatctgcgtaaaaaactggtagactccaccgataaggcggatttacgccttatctatttggcgctggcgcatatgattaagttccgcggtcattttttgatcgagggcgacctgaatccggataacagcgacgttgacaaactgtttatccagctggtgcagacctataaccagttgttcgaagagaacccgattaatgcgtctggcgtggacgcgaaggcgatcctgagcgctcgtctgtccaagtcacgccgcctggagaacttgatcgcgcaactgccgggtgagaagaaaaacggcttgttcggcaatctgattgctctgtccctaggtttgaccccgaatttcaaatctaattttgatctggcggaagatgcgaaattacagttgagcaaggacacttacgatgacgatctggacaacctgctcgctcagatcggcgatcagtatgccgatcttttcctggccgcgaagaacctgagcgatgccatcttgctatcggacatcttacgtgttaacaccgaaatcaccaaagctccgctaagcgcgtccatgattaaacgttacgatgaacaccaccaggatctgacgctgctgaaagcgttggtgcgccagcaactgccggaaaaatacaaggaaattttcttcgaccaaagcaagaacggttatgcaggctatatcgacggtggtgcgtcgcaagaagaattttataaattcatcaaaccgattttggagaagatggatggtaccgaggagctgctggtaaaattgaatcgggaagacctcttgcgaaagcagcgtactttcgataatggtagcatcccgcaccagattcatctgggcgaacttcacgcaatcctacgtcgtcaagaggatttctacccgtttctgaaggataaccgtgaaaagatcgagaagattctgacctttcgtattccgtattatgtgggtccgctggcgagaggcaacagccgttttgcgtggatgacccgcaagtcggaagagactatcacgccgtggaatttcgaggaggtcgtggacaagggtgcatcagcgcaatcttttattgaacgtatgaccaattttgataaaaaccttccaaatgaaaaagtgttgccgaagcacagcttgctgtacgagtatttcaccgtttacaatgaactgacgaaagtcaagtacgttaccgaaggtatgcgtaagccagcctttctgtctggggagcaaaaaaaagccattgtggatctgctgttcaagaccaatcgcaaggttaccgtcaagcaactgaaagaggattacttcaaaaaaatcgaatgttttgactccgtggaaatcagcggcgttgaagaccgctttaatgcatctctgggcacctaccacgatctactcaagatcatcaaggataaggacttcctggacaacgaagaaaatgaggatatcttggaagatattgtgctgactttaacgttgttcgaggatcgtgagatgattgaagaacgcctcaaaacgtacgcacatctgtttgacgataaggtgatgaaacagctaaagcgccgtcgttacaccggttggggtcgtctgtctagaaagctgattaacggcattcgtgacaaacaatctggcaagaccatcctggacttcctcaaatccgacggcttcgcaaatcgtaatttcatgcaactgatccatgatgatagcctgacctttaaagaagatattcagaaggctcaagtgtccggccagggtgactcgctgcatgagcatatcgcgaatctggctggctcccctgcgataaaaaaaggtattctccaaacggtgaaagttgtggatgaactggttaaagtcatgggtcgtcataaaccggaaaacatcgtgattgagatggcacgtgagaaccagaccacccagaaaggtcaaaaaaacagtcgtgagcgtatgaaacgtatcgaagaggggattaaggagttaggcagccagattctgaaggagcacccggttgagaacacccagctgcaaaacgagaagctctacctgtattacctgcaaaacggccgtgacatgtatgttgaccaggagctggacatcaatcgtctcagcgactacgacgtggatgctatcgtgccgcagagctttctaaaagatgattccatcgacaataaagtgctcacccgtagcgacaagaaccgtggtaaaagcgacaacgtgccgagcgaggaagtagttaagaaaatgaaaaactactggcgtcagctattgaacgccaagcttatcacccagcgtaagtttgacaacttgacgaaggcagagcgtggcggcctgagcgagctagacaaggcgggttttattaaacgccagctggtggaaactcgccagattaccaaacacgttgcgcaaatcttggactcacgcatgaatacgaagtacgacgaaaacgataaattgatccgcgaggtgaaggtcataaccctgaaatctaaactggttagcgatttccggaaggactttcaattttataaagtccgggagattaacaactatcaccatgcacacgacgcttacttaaatgcggttgttggcacggccctgatcaagaagtacccgaaactggaaagcgagtttgtttatggtgactacaaggtgtatgacgttcgcaagatgattgcaaaatccgaacaagagattggtaaggcgacggctaagtattttttctacagcaatatcatgaacttcttcaagactgaaatcaccctggcgaacggtgaaattcgtaaacgtccgttgatcgaaaccaacggtgaaaccggtgaaatcgtttgggataaaggtagggattttgcgaccgttcgtaaggtcctgagcatgccgcaggttaacatcgtgaaaaagaccgaagtgcagaccggcggtttctccaaggagagtatcttaccgaagcgcaacagtgacaagctgatcgcgcgtaaaaaagactgggatccgaaaaaatacggcggctttgactccccgaccgtcgcgtacagcgtactggtcgtggctaaagtggagaaaggcaaatcgaaaaagctgaaatccgttaaagagttgctgggtattacgattatggaacgttcgagcttcgagaaaaatccgatcgacttcctggaggccaaaggctacaaggaagttaaaaaagacctgatcattaaactgccgaagtacagcctgttcgagctcgagaacggtcgcaaacgcatgctggcgagcgcgggtgagctccaaaaaggcaacgaattggctctgccgagcaagtacgtgaacttcctgtatctggcctcccattatgaaaaactgaagggtagcccggaagataacgaacaaaagcagctgttcgtggaacaacataaacactatctggacgagattattgagcaaatcagcgagttcagcaaacgcgtgattctggcggatgctaatctggacaaggttctttcagcctataataaacaccgtgataagccgatccgtgaacaggcagaaaatattattcacctgttcaccctgacgaacctgggtgcgccagcagcgtttaaatacttcgacacaacgatcgaccggaagcgttataccagcaccaaagaggttcttgacgccaccttgattcaccaaagcatcacgggtctgtatgaaacccgtattgacttgagccagttgggcggagattcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 14 - DNA, 3342 bp, synthetic construct
  source 1..3342: mol_type = other DNA
atgggaagtcatcaccaccaccaccattcaggtgagaacctgtacttccagagcatggctccgaaaaagaaacgtaaggtgggtatccacggcgttccggcgatggctgattcaagtgaaaaaatgaggggacaatatatcagccgtgacacctttgagaaaaactacaaaccgattgacggcacgaaagaggcgcatctgctgtgcgaaattaaatggggtaagtacggcaagccgtggctgcattggtgtcaaaaccaacgtatgaatatccatgctgaagactacttcatgaacaacatcttcaaagccaaaaagcatccggtgcactgctacgtgacctggtatttgtcctggtcaccgtgcgcggattgtgcgagcaagatcgttaagttcctggaggagcgtccttatctgaaactgaccatttatgttgcacagctgtattatcacactgaagaggagaaccgtaaaggtctgcgtttgctgcgttcgaagaaggtgattatccgcgtgatggatatctctgattacaactactgctggaaagtctttgttagcaatcagaacggtaatgaagactactggccgctgcaattcgatccgtgggttaaggagaactacagccgcctcttggacattttctgggaaagcaagtgccgttccccaaatccgtggtctggaagtgagacacccgggacttcagaatccgcgaccccggagtctggaatgccaaaacccgctgtagagtcagaattttcaaaagttttgaaaaagcactttccgggagaacgtttccgctcgagctatatgaaaagaggcggtaagatcctggctgcccaaggcgaagaggcagttgttgcgtatctacagggcaagtccgaggaggagcctccgaacttccagccaccggcgaagtgccacgtggtgaccaaatcccgtgacttcgcggaatggccgatcatgaaggcgtcggaagcgattcaacgttatatctatgcactgtcgaccaccgaacgcgcggcctgtaaaccgggcaaaagtagtgagagccatgcagcgtggttcgccgctaccggtgtcagcaaccacggctacagccacgttcagggtctgaacctgatttttgatcataccttgggccgttacgatggtgttctgaaaaaggtccaactgcgcaacgaaaaggcgagagcgcgtttagagagcattaacgcgtctagagcagatgaaggtttaccggagattaaagccgaggaggaagaagtggctacgaacgaaaccggccacctgttgcaaccgccgggtatcaatccgagcttttacgtgtatcagaccattagcccgcaggcataccgtccgcgtgacgagatcgtgctgccgccggagtacgcaggttacgtacgtgacccgaatgctccgattccgctgggtgtggtgcgtaaccgttgcgatatccaaaaagggtgcccgggctacatcccggaatggcagcgtgaagcaggtaccgcgatcagcccgaaaacaggcaaggccgtaactgtgccgggcctgtctccgaagaaaaacaaacgtatgcgtaggtactggcgctctgagaaggagaaagctcaagatgcgctgttggttaccgtacgcattggtaccgattgggttgtcatcgacgtgcgcggtctgttgcgcaacgcccgttggcgcaccatcgctcccaaggacatcagcctgaatgcgctcctcgatttgtttaccggtgatccggtgatcgatgtgcgtcgcaatattgttacctttacgtacaccctcgacgcctgtggtacctacgcccgtaaatggaccttgaaaggcaaacagacgaaagcgaccctggacaaactgacggccactcagacggttgcgttggttgcgattgcgcttggtcagaccaacccgatttccgctggcattagccgtgttacacaagaaaatggtgcactgcaatgcgaaccgctggaccggttcaccctgccggatgacctgttgaaggacatcagcgcatatcgcattgcttgggacagaaacgaagaggaactacgcgctcgcagcgtcgaggccctgccggaggcgcaacaagcggaggtccgtgcgctggacggggtgtccaaagaaaccgcgcgtacccaactgtgcgcagatttcggcctggacccgaaacgtctgccgtgggataaaatgagctccaatactacctttatcagcgaagccttgctgtctaacagcgtgagccgtgatcaggtttttttcactccggctccgaagaagggtgctaagaaaaaagcgcctgttgaagttatgcgtaaagaccgcacttgggcacgtgcgtacaagccgcgtctgagcgtcgaagctcagaaactgaagaacgaagccctgtgggcacttaagcgcacctccccggagtatttgaagctgagccgtcgcaaagaggagctgtgtcgtcgtagcatcaactatgttatcgaaaagacccgtcgccgtacgcaatgtcagattgtgatcccggtgatcgaggatctgaatgttcgcttctttcatggttctggtaagagactgcccggttgggataatttcttcaccgctaaaaaagagaaccgttggttcatccagggcctgcacaaagcgtttagcgatctgcgtacgcaccgttccttctatgtttttgaagttcgtccggagcgcacctctattacctgtccaaagtgcggtcattgcgaggtgggtaatcgtgacggtgaggcgtttcagtgcctgagctgcggcaagacgtgcaacgcagatcttgacgtcgcgacccataatctgacccaagtggcgttgaccggtaagaccatgccgaagcgcgaggagccgcgtgacgctcagggtacagcgccagcgcgcaagactaagaaggcgagcaaatcaaaggcgccaccggcggagcgcgaagaccagaccccggcgcaagagccaagccagaccagttctggaggtagcgggggtagcggtggtagcactaacttaagtgatattatcgagaaagaaaccgggaaacagctggtgatccaagaaagcattctgatgctgcccgaagaagttgaagaagtgattggtaacaagcctgagtcggacatcttagtgcacaccgcgtacgatgaaagcacggatgagaatgtgatgctgctcacctctgatgcccccgaatacaagccgtgggctctggtgatccaggactctaacggagaaaacaaaattaagatgctctctggcggctccatggcgcctaaaaaaaaacgcaaagtggggattcacggggttccggcgtaa

SEQ ID NO: 15 - DNA, 4758 bp, synthetic construct
  source 1..4758: mol_type = other DNA
atgggaagtcatcaccaccaccaccattcaggtgagaacctgtacttccagagcatggctccgaaaaagaaacgtaaggtgggtatccacggcgttccggcgatggctgattcaagtgaaaaaatgaggggacaatatatcagccgtgacacctttgagaaaaactacaaaccgattgacggcacgaaagaggcgcatctgctgtgcgaaattaaatggggtaagtacggcaagccgtggctgcattggtgtcaaaaccaacgtatgaatatccatgctgaagactacttcatgaacaacatcttcaaagccaaaaagcatccggtgcactgctacgtgacctggtatttgtcctggtcaccgtgcgcggattgtgcgagcaagatcgttaagttcctggaggagcgtccttatctgaaactgaccatttatgttgcacagctgtattatcacactgaagaggagaaccgtaaaggtctgcgtttgctgcgttcgaagaaggtgattatccgcgtgatggatatctctgattacaactactgctggaaagtctttgttagcaatcagaacggtaatgaagactactggccgctgcaattcgatccgtgggttaaggagaactacagccgcctcttggacattttctgggaaagcaagtgccgttccccaaatccgtggtctggaagtgagacacccgggacttcagaatccgcgaccccggagtctggaatgtcaaaactagaaaagtttacaaattgttactccctgtcgaaaaccctgagattcaaagcgatcccggtgggtaaaactcaagagaacattgataacaaacgtctgctggttgaggacgaaaagcgcgcggaggattacaaaggcgttaaaaagctgttggaccgttattacctgtcctttattaatgatgttctgcacagcattaaattaaagaacctaaacaattacatctccctcttccgtaaaaagacccgcaccgaaaaggaaaataaagaactggagaaccttgagattaatctgcgtaaagaaatcgctaaagcgtttaaaggcaacgagggttataaatccttatttaaaaaagacatcatcgaaacgattctcccagaattcctggacgacaaagacgagatcgcgctggttaattccttcaacggctttaccaccgcattcaccggcttttttgataaccgcgaaaacatgttctctgaagaggccaaaagcacctcaattgcgtttcgctgcattaacgaaaatctgacgcgttatatctctaacatggatatcttcgagaaggttgacgctatctttgataaacatgaagtccaggagatcaaggagaagatcctgaacagcgactacgacgtggaggatttcttcgagggtgagttcttcaattttgttttgacccaagaaggcatcgatgtctacaatgcgataattggtggttttgttaccgagagcggggagaaaattaagggtctgaacgaatatatcaacttgtacaaccagaaaaccaaacaaaaactgccgaagtttaagccgctgtacaagcaagtcttgtcggatcgtgaatcgctgagcttttacggcgaaggctacaccagcgatgaggaggtgttggaggtgttccgcaatacgttgaataagaactctgaaattttttccagcattaagaagctggaaaaactattcaagaacttcgacgagtatagcagcgcaggtatctttgtgaaaaacggtccggctattagcaccatctcgaaagacattttcggcgaatggaatgttattcgtgataagtggaatgccgaatacgacgacattcatctgaagaaaaaggcggttgtaactgaaaagtacgaggacgatcgtcgtaagtcgttcaaaaagatcggctccttctctctggagcaactgcaagagtacgccgatgccgatttgtcagtcgtggagaagctgaaagagatcatcattcaaaaagttgacgaaatctataaggtctatggtagcagtgaaaaactgtttgacgcggacttcgtgctggagaaaagcctgaagaaaaatgacgccgtggtggcaatcatgaaagatcttctggatagcgtcaagtctttcgaaaactacatcaaagcgtttttcggtgaaggcaaggaaacgaatcgtgacgagagcttctatggtgatttcgtcctagcctatgatatcctgcttaaggtggatcacatctatgatgccatccgcaactatgttacccagaaaccgtactcaaaggacaagttcaagttatacttccagaatccgcagtttatgggtggctgggataaagataaggaaaccgactatcgcgccaccattttacgttacggtagtaagtactatctggcaatcatggataaaaaatatgcgaaatgcctgcaaaagatcgacaaggacgatgttaacggcaattatgagaagatcaactacaaactgctgccgggcccaaataagatgctgccgaaggtgtttttcagcaaaaaatggatggcgtattacaatccgagcgaggacatccaaaagatctacaaaaacggtacgtttaagaagggtgatatgttcaacctcaacgattgtcataaacttattgacttcttcaaggatagcatatctcgttatccgaagtggagcaacgcgtatgactttaactttagcgaaaccgagaaatacaaagatatcgcgggtttctatcgtgaagttgaagaacagggttacaaagtgtccttcgagtccgcgagcaagaaggaggtagacaagctggtcgaggagggtaagttgtacatgtttcagatttacaataaggacttttccgacaagtcccatggtacaccgaatttgcacaccatgtatttcaagcttctgttcgacgagaacaaccacggccagatccgtctgagcggtggtgcagagctgttcatgcgtagagctagcctgaagaaagaagagttggtcgttcatccggctaattcgccgatcgcgaacaaaaatccagataaccctaaaaagactacgaccctgagctatgatgtgtataaagacaaacgctttagcgaagaccagtatgagttacacattccgatcgctatcaacaagtgcccgaagaatattttcaagatcaacaccgaagtgcgtgttcttttgaagcacgatgacaacccgtatgtgattggtattgctcgtggtgaacgtaatctgctgtacattgtggttgtggacggcaaaggaaacatcgtggaacagtacagcctgaacgaaatcattaacaatttcaacggcattcgcattaagacggattaccattctttgctggacaaaaaggagaaagagcgtttcgaggcccgtcagaactggacctccatcgagaacattaaggagctgaaggcgggttacatcagccaggtggtgcacaaaatctgcgaactggtggagaagtacgacgcggtgattgctttggcggatttgaactccggttttaagaacagccgcgttaaagtggaaaagcaggtttatcagaaattcgaaaaaatgctgattgataaattgaattacatggttgacaaaaagtctaacccgtgtgcaaccggcggtgcgctgaagggctaccaaatcaccaataagtttgaaagctttaagagcatgagcacgcaaaatggcttcattttttacattccggcgtggctgaccagcaagatcgatccgtcaaccggctttgttaatttactgaagacgaagtatacctctattgccgactctaaaaaatttattagttccttcgatagaatcatgtatgttccggaagaggacctgttcgaatttgcactggactacaagaactttagccgtaccgatgctgattatataaagaaatggaaactctacagctatggcaaccgcatccgtatctttcgcaacccgaaaaaaaacaacgtgttcgactgggaagaggtatgtctgacttcagcgtacaaggagcttttcaataaatatggtatcaactaccagcagggtgatatccgtgcactgctttgcgagcaaagcgataaagcgttctattcaagcttcatggcgctgatgagcctgatgctgcaaatgcgcaacagcatcaccggtcgtaccgatgttgcatttctgatttctccagtaaaaaacagcgatggtatcttttacgacagccgtaattacgaggcccaagaaaatgcgattttgccgaagaacgctgacgcgaatggtgcatacaacattgcaaggaaagttctgtgggcaatcggccaatttaaaaaagcggaagatgagaagctggacaaagtgaaaattgcgattagcaacaaggagtggctggagtatgcgcagacctccgttaaatcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 16 - DNA, 5292 bp, synthetic construct
  source 1..5292: mol_type = other DNA
atgggaagtcatcaccaccaccaccattcaggtgagaacctgtacttccagagcatggctccgaaaaagaaacgtaaggtgggtatccacggcgttccggcgatgagttcagaaacaggacccgttgctgtagacccgacgctgcgccgtcgtattgagccgcacgagttcgaggttttttttgatccgcgtgaactccgcaaggagacatgcttgctgtacgagatcaaatggggcacttcacacaaaatctggcgccacagcagcaagaacaccaccaagcacgtggaagtgaacttcatcgagaaattcacgtctgaacgtcacttttgtccgtccaccagctgtagcattacctggtttctatcctggagcccgtgcggtgaatgttccaaggcgattaccgaattcttgtcgcaacatccgaacgtgactcttgttatttatgtggcccgtttgtaccatcacatggatcagcagaatcgtcaaggcttgcgcgacctggttaacagcggtgttaccatccagatcatgaccgcaccggaatacgactactgctggcgtaatttcgtcaactatccgccgggcaaggaggcgcattggccgcgttatccgccgctgtggatgaaactgtacgctctggaactgcatgcgggtattttaggcctgccaccgtgcttgaatattctgagacgcaagcagcctcagctgacctttttcacgatcgcgctgcaaagctgtcattatcaacgtctgccgccacacatcctgtgggctaccggtctgaaatctggaagtgagacacccgggacttcagaatccgcgaccccggagtctggaatggataaaaagtattcaataggactagctattggcaccaacagcgttggttgggcagttatcaccgatgaatataaggtcccgagcaagaagtttaaggtcctgggcaacaccgatcgccacagcataaaaaagaaccttattggtgcgctgctgttcgattctggtgagactgccgaagcgacccgtctgaagagaaccgcgcgtcgtagatacacccggcgcaagaaccgtatctgctatctgcaagaaattttcagcaacgagatggctaaggtcgacgactccttctttcatcgcctggaggaaagctttctggttgaggaagacaaaaagcacgagcgtcatccgattttcggtaacattgttgatgaagttgcctaccacgaaaaataccccacaatttatcatctgcgtaaaaaactggtagactccaccgataaggcggatttacgccttatctatttggcgctggcgcatatgattaagttccgcggtcattttttgatcgagggcgacctgaatccggataacagcgacgttgacaaactgtttatccagctggtgcagacctataaccagttgttcgaagagaacccgattaatgcgtctggcgtggacgcgaaggcgatcctgagcgctcgtctgtccaagtcacgccgcctggagaacttgatcgcgcaactgccgggtgagaagaaaaacggcttgttcggcaatctgattgctctgtccctaggtttgaccccgaatttcaaatctaattttgatctggcggaagatgcgaaattacagttgagcaaggacacttacgatgacgatctggacaacctgctcgctcagatcggcgatcagtatgccgatcttttcctggccgcgaagaacctgagcgatgccatcttgctatcggacatcttacgtgttaacaccgaaatcaccaaagctccgctaagcgcgtccatgattaaacgttacgatgaacaccaccaggatctgacgctgctgaaagcgttggtgcgccagcaactgccggaaaaatacaaggaaattttcttcgaccaaagcaagaacggttatgcaggctatatcgacggtggtgcgtcgcaagaagaattttataaattcatcaaaccgattttggagaagatggatggtaccgaggagctgctggtaaaattgaatcgggaagacctcttgcgaaagcagcgtactttcgataatggtagcatcccgcaccagattcatctgggcgaacttcacgcaatcctacgtcgtcaagaggatttctacccgtttctgaaggataaccgtgaaaagatcgagaagattctgacctttcgtattccgtattatgtgggtccgctggcgagaggcaacagccgttttgcgtggatgacccgcaagtcggaagagactatcacgccgtggaatttcgaggaggtcgtggacaagggtgcatcagcgcaatcttttattgaacgtatgaccaattttgataaaaaccttccaaatgaaaaagtgttgccgaagcacagcttgctgtacgagtatttcaccgtttacaatgaactgacgaaagtcaagtacgttaccgaaggtatgcgtaagccagcctttctgtctggggagcaaaaaaaagccattgtggatctgctgttcaagaccaatcgcaaggttaccgtcaagcaactgaaagaggattacttcaaaaaaatcgaatgttttgactccgtggaaatcagcggcgttgaagaccgctttaatgcatctctgggcacctaccacgatctactcaagatcatcaaggataaggacttcctggacaacgaagaaaatgaggatatcttggaagatattgtgctgactttaacgttgttcgaggatcgtgagatgattgaagaacgcctcaaaacgtacgcacatctgtttgacgataaggtgatgaaacagctaaagcgccgtcgttacaccggttggggtcgtctgtctagaaagctgattaacggcattcgtgacaaacaatctggcaagaccatcctggacttcctcaaatccgacggcttcgcaaatcgtaatttcatgcaactgatccatgatgatagcctgacctttaaagaagatattcagaaggctcaagtgtccggccagggtgactcgctgcatgagcatatcgcgaatctggctggctcccctgcgataaaaaaaggtattctccaaacggtgaaagttgtggatgaactggttaaagtcatgggtcgtcataaaccggaaaacatcgtgattgagatggcacgtgagaaccagaccacccagaaaggtcaaaaaaacagtcgtgagcgtatgaaacgtatcgaagaggggattaaggagttaggcagccagattctgaaggagcacccggttgagaacacccagctgcaaaacgagaagctctacctgtattacctgcaaaacggccgtgacatgtatgttgaccaggagctggacatcaatcgtctcagcgactacgacgtggatgctatcgtgccgcagagctttctaaaagatgattccatcgacaataaagtgctcacccgtagcgacaagaaccgtggtaaaagcgacaacgtgccgagcgaggaagtagttaagaaaatgaaaaactactggcgtcagctattgaacgccaagcttatcacccagcgtaagtttgacaacttgacgaaggcagagcgtggcggcctgagcgagctagacaaggcgggttttattaaacgccagctggtggaaactcgccagattaccaaacacgttgcgcaaatcttggactcacgcatgaatacgaagtacgacgaaaacgataaattgatccgcgaggtgaaggtcataaccctgaaatctaaactggttagcgatttccggaaggactttcaattttataaagtccgggagattaacaactatcaccatgcacacgacgcttacttaaatgcggttgttggcacggccctgatcaagaagtacccgaaactggaaagcgagtttgtttatggtgactacaaggtgtatgacgttcgcaagatgattgcaaaatccgaacaagagattggtaaggcgacggctaagtattttttctacagcaatatcatgaacttcttcaagactgaaatcaccctggcgaacggtgaaattcgtaaacgtccgttgatcgaaaccaacggtgaaaccggtgaaatcgtttgggataaaggtagggattttgcgaccgttcgtaaggtcctgagcatgccgcaggttaacatcgtgaaaaagaccgaagtgcagaccggcggtttctccaaggagagtatcttaccgaagcgcaacagtgacaagctgatcgcgcgtaaaaaagactgggatccgaaaaaatacggcggctttgactccccgaccgtcgcgtacagcgtactggtcgtggctaaagtggagaaaggcaaatcgaaaaagctgaaatccgttaaagagttgctgggtattacgattatggaacgttcgagcttcgagaaaaatccgatcgacttcctggaggccaaaggctacaaggaagttaaaaaagacctgatcattaaactgccgaagtacagcctgttcgagctcgagaacggtcgcaaacgcatgctggcgagcgcgggtgagctccaaaaaggcaacgaattggctctgccgagcaagtacgtgaacttcctgtatctggcctcccattatgaaaaactgaagggtagcccggaagataacgaacaaaagcagctgttcgtggaacaacataaacactatctggacgagattattgagcaaatcagcgagttcagcaaacgcgtgattctggcggatgctaatctggacaaggttctttcagcctataataaacaccgtgataagccgatccgtgaacaggcagaaaatattattcacctgttcaccctgacgaacctgggtgcgccagcagcgtttaaatacttcgacacaacgatcgaccggaagcgttataccagcaccaaagaggttcttgacgccaccttgattcaccaaagcatcacgggtctgtatgaaacccgtattgacttgagccagttgggcggagattcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 17 - DNA, 3453 bp, synthetic construct
  source 1..3453: mol_type = other DNA
atgggaagtcatcaccaccaccaccattcaggtgagaacctgtacttccagagcatggctccgaaaaagaaacgtaaggtgggtatccacggcgttccggcgatgagttcagaaacaggacccgttgctgtagacccgacgctgcgccgtcgtattgagccgcacgagttcgaggttttttttgatccgcgtgaactccgcaaggagacatgcttgctgtacgagatcaaatggggcacttcacacaaaatctggcgccacagcagcaagaacaccaccaagcacgtggaagtgaacttcatcgagaaattcacgtctgaacgtcacttttgtccgtccaccagctgtagcattacctggtttctatcctggagcccgtgcggtgaatgttccaaggcgattaccgaattcttgtcgcaacatccgaacgtgactcttgttatttatgtggcccgtttgtaccatcacatggatcagcagaatcgtcaaggcttgcgcgacctggttaacagcggtgttaccatccagatcatgaccgcaccggaatacgactactgctggcgtaatttcgtcaactatccgccgggcaaggaggcgcattggccgcgttatccgccgctgtggatgaaactgtacgctctggaactgcatgcgggtattttaggcctgccaccgtgcttgaatattctgagacgcaagcagcctcagctgacctttttcacgatcgcgctgcaaagctgtcattatcaacgtctgccgccacacatcctgtgggctaccggtctgaaatctggaagtgagacacccgggacttcagaatccgcgaccccggagtctggaatgccaaaacccgctgtagagtcagaattttcaaaagttttgaaaaagcactttccgggagaacgtttccgctcgagctatatgaaaagaggcggtaagatcctggctgcccaaggcgaagaggcagttgttgcgtatctacagggcaagtccgaggaggagcctccgaacttccagccaccggcgaagtgccacgtggtgaccaaatcccgtgacttcgcggaatggccgatcatgaaggcgtcggaagcgattcaacgttatatctatgcactgtcgaccaccgaacgcgcggcctgtaaaccgggcaaaagtagtgagagccatgcagcgtggttcgccgctaccggtgtcagcaaccacggctacagccacgttcagggtctgaacctgatttttgatcataccttgggccgttacgatggtgttctgaaaaaggtccaactgcgcaacgaaaaggcgagagcgcgtttagagagcattaacgcgtctagagcagatgaaggtttaccggagattaaagccgaggaggaagaagtggctacgaacgaaaccggccacctgttgcaaccgccgggtatcaatccgagcttttacgtgtatcagaccattagcccgcaggcataccgtccgcgtgacgagatcgtgctgccgccggagtacgcaggttacgtacgtgacccgaatgctccgattccgctgggtgtggtgcgtaaccgttgcgatatccaaaaagggtgcccgggctacatcccggaatggcagcgtgaagcaggtaccgcgatcagcccgaaaacaggcaaggccgtaactgtgccgggcctgtctccgaagaaaaacaaacgtatgcgtaggtactggcgctctgagaaggagaaagctcaagatgcgctgttggttaccgtacgcattggtaccgattgggttgtcatcgacgtgcgcggtctgttgcgcaacgcccgttggcgcaccatcgctcccaaggacatcagcctgaatgcgctcctcgatttgtttaccggtgatccggtgatcgatgtgcgtcgcaatattgttacctttacgtacaccctcgacgcctgtggtacctacgcccgtaaatggaccttgaaaggcaaacagacgaaagcgaccctggacaaactgacggccactcagacggttgcgttggttgcgattgcgcttggtcagaccaacccgatttccgctggcattagccgtgttacacaagaaaatggtgcactgcaatgcgaaccgctggaccggttcaccctgccggatgacctgttgaaggacatcagcgcatatcgcattgcttgggacagaaacgaagaggaactacgcgctcgcagcgtcgaggccctgccggaggcgcaacaagcggaggtccgtgcgctggacggggtgtccaaagaaaccgcgcgtacccaactgtgcgcagatttcggcctggacccgaaacgtctgccgtgggataaaatgagctccaatactacctttatcagcgaagccttgctgtctaacagcgtgagccgtgatcaggtttttttcactccggctccgaagaagggtgctaagaaaaaagcgcctgttgaagttatgcgtaaagaccgcacttgggcacgtgcgtacaagccgcgtctgagcgtcgaagctcagaaactgaagaacgaagccctgtgggcacttaagcgcacctccccggagtatttgaagctgagccgtcgcaaagaggagctgtgtcgtcgtagcatcaactatgttatcgaaaagacccgtcgccgtacgcaatgtcagattgtgatcccggtgatcgaggatctgaatgttcgcttctttcatggttctggtaagagactgcccggttgggataatttcttcaccgctaaaaaagagaaccgttggttcatccagggcctgcacaaagcgtttagcgatctgcgtacgcaccgttccttctatgtttttgaagttcgtccggagcgcacctctattacctgtccaaagtgcggtcattgcgaggtgggtaatcgtgacggtgaggcgtttcagtgcctgagctgcggcaagacgtgcaacgcagatcttgacgtcgcgacccataatctgacccaagtggcgttgaccggtaagaccatgccgaagcgcgaggagccgcgtgacgctcagggtacagcgccagcgcgcaagactaagaaggcgagcaaatcaaaggcgccaccggcggagcgcgaagaccagaccccggcgcaagagccaagccagaccagttctggaggtagcgggggtagcggtggtagcactaacttaagtgatattatcgagaaagaaaccgggaaacagctggtgatccaagaaagcattctgatgctgcccgaagaagttgaagaagtgattggtaacaagcctgagtcggacatcttagtgcacaccgcgtacgatgaaagcacggatgagaatgtgatgctgctcacctctgatgcccccgaatacaagccgtgggctctggtgatccaggactctaacggagaaaacaaaattaagatgctctctggcggctccatggcgcctaaaaaaaaacgcaaagtggggattcacggggttccggcgtaa

SEQ ID NO: 18 - DNA, 4869 bp, synthetic construct
  source 1..4869: mol_type = other DNA
atgggaagtcatcaccaccaccaccattcaggtgagaacctgtacttccagagcatggctccgaaaaagaaacgtaaggtgggtatccacggcgttccggcgatgagttcagaaacaggacccgttgctgtagacccgacgctgcgccgtcgtattgagccgcacgagttcgaggttttttttgatccgcgtgaactccgcaaggagacatgcttgctgtacgagatcaaatggggcacttcacacaaaatctggcgccacagcagcaagaacaccaccaagcacgtggaagtgaacttcatcgagaaattcacgtctgaacgtcacttttgtccgtccaccagctgtagcattacctggtttctatcctggagcccgtgcggtgaatgttccaaggcgattaccgaattcttgtcgcaacatccgaacgtgactcttgttatttatgtggcccgtttgtaccatcacatggatcagcagaatcgtcaaggcttgcgcgacctggttaacagcggtgttaccatccagatcatgaccgcaccggaatacgactactgctggcgtaatttcgtcaactatccgccgggcaaggaggcgcattggccgcgttatccgccgctgtggatgaaactgtacgctctggaactgcatgcgggtattttaggcctgccaccgtgcttgaatattctgagacgcaagcagcctcagctgacctttttcacgatcgcgctgcaaagctgtcattatcaacgtctgccgccacacatcctgtgggctaccggtctgaaatctggaagtgagacacccgggacttcagaatccgcgaccccggagtctggaatgtcaaaactagaaaagtttacaaattgttactccctgtcgaaaaccctgagattcaaagcgatcccggtgggtaaaactcaagagaacattgataacaaacgtctgctggttgaggacgaaaagcgcgcggaggattacaaaggcgttaaaaagctgttggaccgttattacctgtcctttattaatgatgttctgcacagcattaaattaaagaacctaaacaattacatctccctcttccgtaaaaagacccgcaccgaaaaggaaaataaagaactggagaaccttgagattaatctgcgtaaagaaatcgctaaagcgtttaaaggcaacgagggttataaatccttatttaaaaaagacatcatcgaaacgattctcccagaattcctggacgacaaagacgagatcgcgctggttaattccttcaacggctttaccaccgcattcaccggcttttttgataaccgcgaaaacatgttctctgaagaggccaaaagcacctcaattgcgtttcgctgcattaacgaaaatctgacgcgttatatctctaacatggatatcttcgagaaggttgacgctatctttgataaacatgaagtccaggagatcaaggagaagatcctgaacagcgactacgacgtggaggatttcttcgagggtgagttcttcaattttgttttgacccaagaaggcatcgatgtctacaatgcgataattggtggttttgttaccgagagcggggagaaaattaagggtctgaacgaatatatcaacttgtacaaccagaaaaccaaacaaaaactgccgaagtttaagccgctgtacaagcaagtcttgtcggatcgtgaatcgctgagcttttacggcgaaggctacaccagcgatgaggaggtgttggaggtgttccgcaatacgttgaataagaactctgaaattttttccagcattaagaagctggaaaaactattcaagaacttcgacgagtatagcagcgcaggtatctttgtgaaaaacggtccggctattagcaccatctcgaaagacattttcggcgaatggaatgttattcgtgataagtggaatgccgaatacgacgacattcatctgaagaaaaaggcggttgtaactgaaaagtacgaggacgatcgtcgtaagtcgttcaaaaagatcggctccttctctctggagcaactgcaagagtacgccgatgccgatttgtcagtcgtggagaagctgaaagagatcatcattcaaaaagttgacgaaatctataaggtctatggtagcagtgaaaaactgtttgacgcggacttcgtgctggagaaaagcctgaagaaaaatgacgccgtggtggcaatcatgaaagatcttctggatagcgtcaagtctttcgaaaactacatcaaagcgtttttcggtgaaggcaaggaaacgaatcgtgacgagagcttctatggtgatttcgtcctagcctatgatatcctgcttaaggtggatcacatctatgatgccatccgcaactatgttacccagaaaccgtactcaaaggacaagttcaagttatacttccagaatccgcagtttatgggtggctgggataaagataaggaaaccgactatcgcgccaccattttacgttacggtagtaagtactatctggcaatcatggataaaaaatatgcgaaatgcctgcaaaagatcgacaaggacgatgttaacggcaattatgagaagatcaactacaaactgctgccgggcccaaataagatgctgccgaaggtgtttttcagcaaaaaatggatggcgtattacaatccgagcgaggacatccaaaagatctacaaaaacggtacgtttaagaagggtgatatgttcaacctcaacgattgtcataaacttattgacttcttcaaggatagcatatctcgttatccgaagtggagcaacgcgtatgactttaactttagcgaaaccgagaaatacaaagatatcgcgggtttctatcgtgaagttgaagaacagggttacaaagtgtccttcgagtccgcgagcaagaaggaggtagacaagctggtcgaggagggtaagttgtacatgtttcagatttacaataaggacttttccgacaagtcccatggtacaccgaatttgcacaccatgtatttcaagcttctgttcgacgagaacaaccacggccagatccgtctgagcggtggtgcagagctgttcatgcgtagagctagcctgaagaaagaagagttggtcgttcatccggctaattcgccgatcgcgaacaaaaatccagataaccctaaaaagactacgaccctgagctatgatgtgtataaagacaaacgctttagcgaagaccagtatgagttacacattccgatcgctatcaacaagtgcccgaagaatattttcaagatcaacaccgaagtgcgtgttcttttgaagcacgatgacaacccgtatgtgattggtattgctcgtggtgaacgtaatctgctgtacattgtggttgtggacggcaaaggaaacatcgtggaacagtacagcctgaacgaaatcattaacaatttcaacggcattcgcattaagacggattaccattctttgctggacaaaaaggagaaagagcgtttcgaggcccgtcagaactggacctccatcgagaacattaaggagctgaaggcgggttacatcagccaggtggtgcacaaaatctgcgaactggtggagaagtacgacgcggtgattgctttggcggatttgaactccggttttaagaacagccgcgttaaagtggaaaagcaggtttatcagaaattcgaaaaaatgctgattgataaattgaattacatggttgacaaaaagtctaacccgtgtgcaaccggcggtgcgctgaagggctaccaaatcaccaataagtttgaaagctttaagagcatgagcacgcaaaatggcttcattttttacattccggcgtggctgaccagcaagatcgatccgtcaaccggctttgttaatttactgaagacgaagtatacctctattgccgactctaaaaaatttattagttccttcgatagaatcatgtatgttccggaagaggacctgttcgaatttgcactggactacaagaactttagccgtaccgatgctgattatataaagaaatggaaactctacagctatggcaaccgcatccgtatctttcgcaacccgaaaaaaaacaacgtgttcgactgggaagaggtatgtctgacttcagcgtacaaggagcttttcaataaatatggtatcaactaccagcagggtgatatccgtgcactgctttgcgagcaaagcgataaagcgttctattcaagcttcatggcgctgatgagcctgatgctgcaaatgcgcaacagcatcaccggtcgtaccgatgttgcatttctgatttctccagtaaaaaacagcgatggtatcttttacgacagccgtaattacgaggcccaagaaaatgcgattttgccgaagaacgctgacgcgaatggtgcatacaacattgcaaggaaagttctgtgggcaatcggccaatttaaaaaagcggaagatgagaagctggacaaagtgaaaattgcgattagcaacaaggagtggctggagtatgcgcagacctccgttaaatcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 19 - DNA, 5202 bp, synthetic construct
  source 1..5202: mol_type = other DNA
atgggaagtcatcaccaccaccaccattcaggtgagaacctgtacttccagagcatggctccgaaaaagaaacgtaaggtgggtatccacggcgttccggcgatggaagcaagtcccgcttcaggaccaaggcacctgatggacccacatattttcaccagtaattttaacaacggcatcggccgtcacaagacctacctgtgttatgaggtcgaacgtctggataatggcacttccgtgaaaatggatcagcatcgtggtttcctgcatggtcaggctaagaatctgctttgcggtttctatggtcgccacgccgaactgcgttttttggacctggttccgtccctccaactggacccggcgcaaatctatcgtgttacctggtttatcagctggtcgccgtgtttcagctggggttgcgcaggcgaagtacgtgcgttcttacaagagaacacccatgtgcgcttgcgcatcttcgccgcacgtatttacgactacgacccgctgtataaagaggcgttgcaaatgctgcgcgatgctggcgcacaggttagcattatgacgtacgacgaatttaagcactgctgggataccttcgtggatcaccagggctgcccgtttcagccgtgggatggtttggacgagcactctcaggcgctgagcggtcgtctcagagcgattctgcaaaaccagggaaactctggaagtgagacacccgggacttcagaatccgcgaccccggagtctggaatggataaaaagtattcaataggactagctattggcaccaacagcgttggttgggcagttatcaccgatgaatataaggtcccgagcaagaagtttaaggtcctgggcaacaccgatcgccacagcataaaaaagaaccttattggtgcgctgctgttcgattctggtgagactgccgaagcgacccgtctgaagagaaccgcgcgtcgtagatacacccggcgcaagaaccgtatctgctatctgcaagaaattttcagcaacgagatggctaaggtcgacgactccttctttcatcgcctggaggaaagctttctggttgaggaagacaaaaagcacgagcgtcatccgattttcggtaacattgttgatgaagttgcctaccacgaaaaataccccacaatttatcatctgcgtaaaaaactggtagactccaccgataaggcggatttacgccttatctatttggcgctggcgcatatgattaagttccgcggtcattttttgatcgagggcgacctgaatccggataacagcgacgttgacaaactgtttatccagctggtgcagacctataaccagttgttcgaagagaacccgattaatgcgtctggcgtggacgcgaaggcgatcctgagcgctcgtctgtccaagtcacgccgcctggagaacttgatcgcgcaactgccgggtgagaagaaaaacggcttgttcggcaatctgattgctctgtccctaggtttgaccccgaatttcaaatctaattttgatctggcggaagatgcgaaattacagttgagcaaggacacttacgatgacgatctggacaacctgctcgctcagatcggcgatcagtatgccgatcttttcctggccgcgaagaacctgagcgatgccatcttgctatcggacatcttacgtgttaacaccgaaatcaccaaagctccgctaagcgcgtccatgattaaacgttacgatgaacaccaccaggatctgacgctgctgaaagcgttggtgcgccagcaactgccggaaaaatacaaggaaattttcttcgaccaaagcaagaacggttatgcaggctatatcgacggtggtgcgtcgcaagaagaattttataaattcatcaaaccgattttggagaagatggatggtaccgaggagctgctggtaaaattgaatcgggaagacctcttgcgaaagcagcgtactttcgataatggtagcatcccgcaccagattcatctgggcgaacttcacgcaatcctacgtcgtcaagaggatttctacccgtttctgaaggataaccgtgaaaagatcgagaagattctgacctttcgtattccgtattatgtgggtccgctggcgagaggcaacagccgttttgcgtggatgacccgcaagtcggaagagactatcacgccgtggaatttcgaggaggtcgtggacaagggtgcatcagcgcaatcttttattgaacgtatgaccaattttgataaaaaccttccaaatgaaaaagtgttgccgaagcacagcttgctgtacgagtatttcaccgtttacaatgaactgacgaaagtcaagtacgttaccgaaggtatgcgtaagccagcctttctgtctggggagcaaaaaaaagccattgtggatctgctgttcaagaccaatcgcaaggttaccgtcaagcaactgaaagaggattacttcaaaaaaatcgaatgttttgactccgtggaaatcagcggcgttgaagaccgctttaatgcatctctgggcacctaccacgatctactcaagatcatcaaggataaggacttcctggacaacgaagaaaatgaggatatcttggaagatattgtgctgactttaacgttgttcgaggatcgtgagatgattgaagaacgcctcaaaacgtacgcacatctgtttgacgataaggtgatgaaacagctaaagcgccgtcgttacaccggttggggtcgtctgtctagaaagctgattaacggcattcgtgacaaacaatctggcaagaccatcctggacttcctcaaatccgacggcttcgcaaatcgtaatttcatgcaactgatccatgatgatagcctgacctttaaagaagatattcagaaggctcaagtgtccggccagggtgactcgctgcatgagcatatcgcgaatctggctggctcccctgcgataaaaaaaggtattctccaaacggtgaaagttgtggatgaactggttaaagtcatgggtcgtcataaaccggaaaacatcgtgattgagatggcacgtgagaaccagaccacccagaaaggtcaaaaaaacagtcgtgagcgtatgaaacgtatcgaagaggggattaaggagttaggcagccagattctgaaggagcacccggttgagaacacccagctgcaaaacgagaagctctacctgtattacctgcaaaacggccgtgacatgtatgttgaccaggagctggacatcaatcgtctcagcgactacgacgtggatgctatcgtgccgcagagctttctaaaagatgattccatcgacaataaagtgctcacccgtagcgacaagaaccgtggtaaaagcgacaacgtgccgagcgaggaagtagttaagaaaatgaaaaactactggcgtcagctattgaacgccaagcttatcacccagcgtaagtttgacaacttgacgaaggcagagcgtggcggcctgagcgagctagacaaggcgggttttattaaacgccagctggtggaaactcgccagattaccaaacacgttgcgcaaatcttggactcacgcatgaatacgaagtacgacgaaaacgataaattgatccgcgaggtgaaggtcataaccctgaaatctaaactggttagcgatttccggaaggactttcaattttataaagtccgggagattaacaactatcaccatgcacacgacgcttacttaaatgcggttgttggcacggccctgatcaagaagtacccgaaactggaaagcgagtttgtttatggtgactacaaggtgtatgacgttcgcaagatgattgcaaaatccgaacaagagattggtaaggcgacggctaagtattttttctacagcaatatcatgaacttcttcaagactgaaatcaccctggcgaacggtgaaattcgtaaacgtccgttgatcgaaaccaacggtgaaaccggtgaaatcgtttgggataaaggtagggattttgcgaccgttcgtaaggtcctgagcatgccgcaggttaacatcgtgaaaaagaccgaagtgcagaccggcggtttctccaaggagagtatcttaccgaagcgcaacagtgacaagctgatcgcgcgtaaaaaagactgggatccgaaaaaatacggcggctttgactccccgaccgtcgcgtacagcgtactggtcgtggctaaagtggagaaaggcaaatcgaaaaagctgaaatccgttaaagagttgctgggtattacgattatggaacgttcgagcttcgagaaaaatccgatcgacttcctggaggccaaaggctacaaggaagttaaaaaagacctgatcattaaactgccgaagtacagcctgttcgagctcgagaacggtcgcaaacgcatgctggcgagcgcgggtgagctccaaaaaggcaacgaattggctctgccgagcaagtacgtgaacttcctgtatctggcctcccattatgaaaaactgaagggtagcccggaagataacgaacaaaagcagctgttcgtggaacaacataaacactatctggacgagattattgagcaaatcagcgagttcagcaaacgcgtgattctggcggatgctaatctggacaaggttctttcagcctataataaacaccgtgataagccgatccgtgaacaggcagaaaatattattcacctgttcaccctgacgaacctgggtgcgccagcagcgtttaaatacttcgacacaacgatcgaccggaagcgttataccagcaccaaagaggttcttgacgccaccttgattcaccaaagcatcacgggtctgtatgaaacccgtattgacttgagccagttgggcggagattcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 20 - DNA, 3363 bp, synthetic construct
  source 1..3363: mol_type = other DNA
atgggaagtcatcaccaccaccaccattcaggtgagaacctgtacttccagagcatggctccgaaaaagaaacgtaaggtgggtatccacggcgttccggcgatggaagcaagtcccgcttcaggaccaaggcacctgatggacccacatattttcaccagtaattttaacaacggcatcggccgtcacaagacctacctgtgttatgaggtcgaacgtctggataatggcacttccgtgaaaatggatcagcatcgtggtttcctgcatggtcaggctaagaatctgctttgcggtttctatggtcgccacgccgaactgcgttttttggacctggttccgtccctccaactggacccggcgcaaatctatcgtgttacctggtttatcagctggtcgccgtgtttcagctggggttgcgcaggcgaagtacgtgcgttcttacaagagaacacccatgtgcgcttgcgcatcttcgccgcacgtatttacgactacgacccgctgtataaagaggcgttgcaaatgctgcgcgatgctggcgcacaggttagcattatgacgtacgacgaatttaagcactgctgggataccttcgtggatcaccagggctgcccgtttcagccgtgggatggtttggacgagcactctcaggcgctgagcggtcgtctcagagcgattctgcaaaaccagggaaactctggaagtgagacacccgggacttcagaatccgcgaccccggagtctggaatgccaaaacccgctgtagagtcagaattttcaaaagttttgaaaaagcactttccgggagaacgtttccgctcgagctatatgaaaagaggcggtaagatcctggctgcccaaggcgaagaggcagttgttgcgtatctacagggcaagtccgaggaggagcctccgaacttccagccaccggcgaagtgccacgtggtgaccaaatcccgtgacttcgcggaatggccgatcatgaaggcgtcggaagcgattcaacgttatatctatgcactgtcgaccaccgaacgcgcggcctgtaaaccgggcaaaagtagtgagagccatgcagcgtggttcgccgctaccggtgtcagcaaccacggctacagccacgttcagggtctgaacctgatttttgatcataccttgggccgttacgatggtgttctgaaaaaggtccaactgcgcaacgaaaaggcgagagcgcgtttagagagcattaacgcgtctagagcagatgaaggtttaccggagattaaagccgaggaggaagaagtggctacgaacgaaaccggccacctgttgcaaccgccgggtatcaatccgagcttttacgtgtatcagaccattagcccgcaggcataccgtccgcgtgacgagatcgtgctgccgccggagtacgcaggttacgtacgtgacccgaatgctccgattccgctgggtgtggtgcgtaaccgttgcgatatccaaaaagggtgcccgggctacatcccggaatggcagcgtgaagcaggtaccgcgatcagcccgaaaacaggcaaggccgtaactgtgccgggcctgtctccgaagaaaaacaaacgtatgcgtaggtactggcgctctgagaaggagaaagctcaagatgcgctgttggttaccgtacgcattggtaccgattgggttgtcatcgacgtgcgcggtctgttgcgcaacgcccgttggcgcaccatcgctcccaaggacatcagcctgaatgcgctcctcgatttgtttaccggtgatccggtgatcgatgtgcgtcgcaatattgttacctttacgtacaccctcgacgcctgtggtacctacgcccgtaaatggaccttgaaaggcaaacagacgaaagcgaccctggacaaactgacggccactcagacggttgcgttggttgcgattgcgcttggtcagaccaacccgatttccgctggcattagccgtgttacacaagaaaatggtgcactgcaatgcgaaccgctggaccggttcaccctgccggatgacctgttgaaggacatcagcgcatatcgcattgcttgggacagaaacgaagaggaactacgcgctcgcagcgtcgaggccctgccggaggcgcaacaagcggaggtccgtgcgctggacggggtgtccaaagaaaccgcgcgtacccaactgtgcgcagatttcggcctggacccgaaacgtctgccgtgggataaaatgagctccaatactacctttatcagcgaagccttgctgtctaacagcgtgagccgtgatcaggtttttttcactccggctccgaagaagggtgctaagaaaaaagcgcctgttgaagttatgcgtaaagaccgcacttgggcacgtgcgtacaagccgcgtctgagcgtcgaagctcagaaactgaagaacgaagccctgtgggcacttaagcgcacctccccggagtatttgaagctgagccgtcgcaaagaggagctgtgtcgtcgtagcatcaactatgttatcgaaaagacccgtcgccgtacgcaatgtcagattgtgatcccggtgatcgaggatctgaatgttcgcttctttcatggttctggtaagagactgcccggttgggataatttcttcaccgctaaaaaagagaaccgttggttcatccagggcctgcacaaagcgtttagcgatctgcgtacgcaccgttccttctatgtttttgaagttcgtccggagcgcacctctattacctgtccaaagtgcggtcattgcgaggtgggtaatcgtgacggtgaggcgtttcagtgcctgagctgcggcaagacgtgcaacgcagatcttgacgtcgcgacccataatctgacccaagtggcgttgaccggtaagaccatgccgaagcgcgaggagccgcgtgacgctcagggtacagcgccagcgcgcaagactaagaaggcgagcaaatcaaaggcgccaccggcggagcgcgaagaccagaccccggcgcaagagccaagccagaccagttctggaggtagcgggggtagcggtggtagcactaacttaagtgatattatcgagaaagaaaccgggaaacagctggtgatccaagaaagcattctgatgctgcccgaagaagttgaagaagtgattggtaacaagcctgagtcggacatcttagtgcacaccgcgtacgatgaaagcacggatgagaatgtgatgctgctcacctctgatgcccccgaatacaagccgtgggctctggtgatccaggactctaacggagaaaacaaaattaagatgctctctggcggctccatggcgcctaaaaaaaaacgcaaagtggggattcacggggttccggcgtaa

SEQ ID NO: 21 - DNA, 4779 bp, synthetic construct
  source 1..4779: mol_type = other DNA
atgggaagtcatcaccaccaccaccattcaggtgagaacctgtacttccagagcatggctccgaaaaagaaacgtaaggtgggtatccacggcgttccggcgatggaagcaagtcccgcttcaggaccaaggcacctgatggacccacatattttcaccagtaattttaacaacggcatcggccgtcacaagacctacctgtgttatgaggtcgaacgtctggataatggcacttccgtgaaaatggatcagcatcgtggtttcctgcatggtcaggctaagaatctgctttgcggtttctatggtcgccacgccgaactgcgttttttggacctggttccgtccctccaactggacccggcgcaaatctatcgtgttacctggtttatcagctggtcgccgtgtttcagctggggttgcgcaggcgaagtacgtgcgttcttacaagagaacacccatgtgcgcttgcgcatcttcgccgcacgtatttacgactacgacccgctgtataaagaggcgttgcaaatgctgcgcgatgctggcgcacaggttagcattatgacgtacgacgaatttaagcactgctgggataccttcgtggatcaccagggctgcccgtttcagccgtgggatggtttggacgagcactctcaggcgctgagcggtcgtctcagagcgattctgcaaaaccagggaaactctggaagtgagacacccgggacttcagaatccgcgaccccggagtctggaatgtcaaaactagaaaagtttacaaattgttactccctgtcgaaaaccctgagattcaaagcgatcccggtgggtaaaactcaagagaacattgataacaaacgtctgctggttgaggacgaaaagcgcgcggaggattacaaaggcgttaaaaagctgttggaccgttattacctgtcctttattaatgatgttctgcacagcattaaattaaagaacctaaacaattacatctccctcttccgtaaaaagacccgcaccgaaaaggaaaataaagaactggagaaccttgagattaatctgcgtaaagaaatcgctaaagcgtttaaaggcaacgagggttataaatccttatttaaaaaagacatcatcgaaacgattctcccagaattcctggacgacaaagacgagatcgcgctggttaattccttcaacggctttaccaccgcattcaccggcttttttgataaccgcgaaaacatgttctctgaagaggccaaaagcacctcaattgcgtttcgctgcattaacgaaaatctgacgcgttatatctctaacatggatatcttcgagaaggttgacgctatctttgataaacatgaagtccaggagatcaaggagaagatcctgaacagcgactacgacgtggaggatttcttcgagggtgagttcttcaattttgttttgacccaagaaggcatcgatgtctacaatgcgataattggtggttttgttaccgagagcggggagaaaattaagggtctgaacgaatatatcaacttgtacaaccagaaaaccaaacaaaaactgccgaagtttaagccgctgtacaagcaagtcttgtcggatcgtgaatcgctgagcttttacggcgaaggctacaccagcgatgaggaggtgttggaggtgttccgcaatacgttgaataagaactctgaaattttttccagcattaagaagctggaaaaactattcaagaacttcgacgagtatagcagcgcaggtatctttgtgaaaaacggtccggctattagcaccatctcgaaagacattttcggcgaatggaatgttattcgtgataagtggaatgccgaatacgacgacattcatctgaagaaaaaggcggttgtaactgaaaagtacgaggacgatcgtcgtaagtcgttcaaaaagatcggctccttctctctggagcaactgcaagagtacgccgatgccgatttgtcagtcgtggagaagctgaaagagatcatcattcaaaaagttgacgaaatctataaggtctatggtagcagtgaaaaactgtttgacgcggacttcgtgctggagaaaagcctgaagaaaaatgacgccgtggtggcaatcatgaaagatcttctggatagcgtcaagtctttcgaaaactacatcaaagcgtttttcggtgaaggcaaggaaacgaatcgtgacgagagcttctatggtgatttcgtcctagcctatgatatcctgcttaaggtggatcacatctatgatgccatccgcaactatgttacccagaaaccgtactcaaaggacaagttcaagttatacttccagaatccgcagtttatgggtggctgggataaagataaggaaaccgactatcgcgccaccattttacgttacggtagtaagtactatctggcaatcatggataaaaaatatgcgaaatgcctgcaaaagatcgacaaggacgatgttaacggcaattatgagaagatcaactacaaactgctgccgggcccaaataagatgctgccgaaggtgtttttcagcaaaaaatggatggcgtattacaatccgagcgaggacatccaaaagatctacaaaaacggtacgtttaagaagggtgatatgttcaacctcaacgattgtcataaacttattgacttcttcaaggatagcatatctcgttatccgaagtggagcaacgcgtatgactttaactttagcgaaaccgagaaatacaaagatatcgcgggtttctatcgtgaagttgaagaacagggttacaaagtgtccttcgagtccgcgagcaagaaggaggtagacaagctggtcgaggagggtaagttgtacatgtttcagatttacaataaggacttttccgacaagtcccatggtacaccgaatttgcacaccatgtatttcaagcttctgttcgacgagaacaaccacggccagatccgtctgagcggtggtgcagagctgttcatgcgtagagctagcctgaagaaagaagagttggtcgttcatccggctaattcgccgatcgcgaacaaaaatccagataaccctaaaaagactacgaccctgagctatgatgtgtataaagacaaacgctttagcgaagaccagtatgagttacacattccgatcgctatcaacaagtgcccgaagaatattttcaagatcaacaccgaagtgcgtgttcttttgaagcacgatgacaacccgtatgtgattggtattgctcgtggtgaacgtaatctgctgtacattgtggttgtggacggcaaaggaaacatcgtggaacagtacagcctgaacgaaatcattaacaatttcaacggcattcgcattaagacggattaccattctttgctggacaaaaaggagaaagagcgtttcgaggcccgtcagaactggacctccatcgagaacattaaggagctgaaggcgggttacatcagccaggtggtgcacaaaatctgcgaactggtggagaagtacgacgcggtgattgctttggcggatttgaactccggttttaagaacagccgcgttaaagtggaaaagcaggtttatcagaaattcgaaaaaatgctgattgataaattgaattacatggttgacaaaaagtctaacccgtgtgcaaccggcggtgcgctgaagggctaccaaatcaccaataagtttgaaagctttaagagcatgagcacgcaaaatggcttcattttttacattccggcgtggctgaccagcaagatcgatccgtcaaccggctttgttaatttactgaagacgaagtatacctctattgccgactctaaaaaatttattagttccttcgatagaatcatgtatgttccggaagaggacctgttcgaatttgcactggactacaagaactttagccgtaccgatgctgattatataaagaaatggaaactctacagctatggcaaccgcatccgtatctttcgcaacccgaaaaaaaacaacgtgttcgactgggaagaggtatgtctgacttcagcgtacaaggagcttttcaataaatatggtatcaactaccagcagggtgatatccgtgcactgctttgcgagcaaagcgataaagcgttctattcaagcttcatggcgctgatgagcctgatgctgcaaatgcgcaacagcatcaccggtcgtaccgatgttgcatttctgatttctccagtaaaaaacagcgatggtatcttttacgacagccgtaattacgaggcccaagaaaatgcgattttgccgaagaacgctgacgcgaatggtgcatacaacattgcaaggaaagttctgtgggcaatcggccaatttaaaaaagcggaagatgagaagctggacaaagtgaaaattgcgattagcaacaaggagtggctggagtatgcgcagacctccgttaaatcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 22 - DNA, 5361 bp, synthetic construct
  source 1..5361: mol_type = other DNA
atgggaagtcatcaccaccaccaccattcaggtgagaacctgtacttccagagcatggctccgaaaaagaaacgtaaggtgggtatccacggcgttccggcgatgtcaagtaaaacaggacccgttgctgtagatccgacgctgagacgtcgcatcgagccgcacgaattcgaggtgttttttgatccgcgtgaactgcgcaaggagacgtgcctgctgtatgagatcaactggggtggccgtcacagcatttggcgtcacacctcccaaaacacgaacaaacacgtggaagtcaatttcatcgaaaaattcaccaccgaacgttacttctgcccgaacacccgttgttccatcacctggttcctgtcttggagcccgtgtggtgagtgcagccgcgcaattactgagttcctgagccgttacccgaatgttaccctgtttatctatatcgcccgtttgtaccatctggcgaacccgcgcaaccgtcagggtttacgcgacttaattagctctggcgttaccattcagattatgaccgagcaagagagcggttattgctggcataactttgtaaactatagcccgagcaatgaaagccattggcctcgctacccgcacctgtgggttcgtttgtacgtgctggaactgtactgcatcatccttggtttgccaccgtgcttgaatattttgagacgtaagcagtcgcaactgaccagttttacgatcgcgctacagagctgtcattatcagcgtctcccgccacacattctgtgggctaccggcctgaaaagcggcggtagcagcggtggctccagcggctcggaaactccgggcacctccgagtcagcgaccccggaatctggaagtgagacacccgggacttcagaatccgcgaccccggagtctggaatggataaaaagtattcaataggactagctattggcaccaacagcgttggttgggcagttatcaccgatgaatataaggtcccgagcaagaagtttaaggtcctgggcaacaccgatcgccacagcataaaaaagaaccttattggtgcgctgctgttcgattctggtgagactgccgaagcgacccgtctgaagagaaccgcgcgtcgtagatacacccggcgcaagaaccgtatctgctatctgcaagaaattttcagcaacgagatggctaaggtcgacgactccttctttcatcgcctggaggaaagctttctggttgaggaagacaaaaagcacgagcgtcatccgattttcggtaacattgttgatgaagttgcctaccacgaaaaataccccacaatttatcatctgcgtaaaaaactggtagactccaccgataaggcggatttacgccttatctatttggcgctggcgcatatgattaagttccgcggtcattttttgatcgagggcgacctgaatccggataacagcgacgttgacaaactgtttatccagctggtgcagacctataaccagttgttcgaagagaacccgattaatgcgtctggcgtggacgcgaaggcgatcctgagcgctcgtctgtccaagtcacgccgcctggagaacttgatcgcgcaactgccgggtgagaagaaaaacggcttgttcggcaatctgattgctctgtccctaggtttgaccccgaatttcaaatctaattttgatctggcggaagatgcgaaattacagttgagcaaggacacttacgatgacgatctggacaacctgctcgctcagatcggcgatcagtatgccgatcttttcctggccgcgaagaacctgagcgatgccatcttgctatcggacatcttacgtgttaacaccgaaatcaccaaagctccgctaagcgcgtccatgattaaacgttacgatgaacaccaccaggatctgacgctgctgaaagcgttggtgcgccagcaactgccggaaaaatacaaggaaattttcttcgaccaaagcaagaacggttatgcaggctatatcgacggtggtgcgtcgcaagaagaattttataaattcatcaaaccgattttggagaagatggatggtaccgaggagctgctggtaaaattgaatcgggaagacctcttgcgaaagcagcgtactttcgataatggtagcatcccgcaccagattcatctgggcgaacttcacgcaatcctacgtcgtcaagaggatttctacccgtttctgaaggataaccgtgaaaagatcgagaagattctgacctttcgtattccgtattatgtgggtccgctggcgagaggcaacagccgttttgcgtggatgacccgcaagtcggaagagactatcacgccgtggaatttcgaggaggtcgtggacaagggtgcatcagcgcaatcttttattgaacgtatgaccaattttgataaaaaccttccaaatgaaaaagtgttgccgaagcacagcttgctgtacgagtatttcaccgtttacaatgaactgacgaaagtcaagtacgttaccgaaggtatgcgtaagccagcctttctgtctggggagcaaaaaaaagccattgtggatctgctgttcaagaccaatcgcaaggttaccgtcaagcaactgaaagaggattacttcaaaaaaatcgaatgttttgactccgtggaaatcagcggcgttgaagaccgctttaatgcatctctgggcacctaccacgatctactcaagatcatcaaggataaggacttcctggacaacgaagaaaatgaggatatcttggaagatattgtgctgactttaacgttgttcgaggatcgtgagatgattgaagaacgcctcaaaacgtacgcacatctgtttgacgataaggtgatgaaacagctaaagcgccgtcgttacaccggttggggtcgtctgtctagaaagctgattaacggcattcgtgacaaacaatctggcaagaccatcctggacttcctcaaatccgacggcttcgcaaatcgtaatttcatgcaactgatccatgatgatagcctgacctttaaagaagatattcagaaggctcaagtgtccggccagggtgactcgctgcatgagcatatcgcgaatctggctggctcccctgcgataaaaaaaggtattctccaaacggtgaaagttgtggatgaactggttaaagtcatgggtcgtcataaaccggaaaacatcgtgattgagatggcacgtgagaaccagaccacccagaaaggtcaaaaaaacagtcgtgagcgtatgaaacgtatcgaagaggggattaaggagttaggcagccagattctgaaggagcacccggttgagaacacccagctgcaaaacgagaagctctacctgtattacctgcaaaacggccgtgacatgtatgttgaccaggagctggacatcaatcgtctcagcgactacgacgtggatgctatcgtgccgcagagctttctaaaagatgattccatcgacaataaagtgctcacccgtagcgacaagaaccgtggtaaaagcgacaacgtgccgagcgaggaagtagttaagaaaatgaaaaactactggcgtcagctattgaacgccaagcttatcacccagcgtaagtttgacaacttgacgaaggcagagcgtggcggcctgagcgagctagacaaggcgggttttattaaacgccagctggtggaaactcgccagattaccaaacacgttgcgcaaatcttggactcacgcatgaatacgaagtacgacgaaaacgataaattgatccgcgaggtgaaggtcataaccctgaaatctaaactggttagcgatttccggaaggactttcaattttataaagtccgggagattaacaactatcaccatgcacacgacgcttacttaaatgcggttgttggcacggccctgatcaagaagtacccgaaactggaaagcgagtttgtttatggtgactacaaggtgtatgacgttcgcaagatgattgcaaaatccgaacaagagattggtaaggcgacggctaagtattttttctacagcaatatcatgaacttcttcaagactgaaatcaccctggcgaacggtgaaattcgtaaacgtccgttgatcgaaaccaacggtgaaaccggtgaaatcgtttgggataaaggtagggattttgcgaccgttcgtaaggtcctgagcatgccgcaggttaacatcgtgaaaaagaccgaagtgcagaccggcggtttctccaaggagagtatcttaccgaagcgcaacagtgacaagctgatcgcgcgtaaaaaagactgggatccgaaaaaatacggcggctttgactccccgaccgtcgcgtacagcgtactggtcgtggctaaagtggagaaaggcaaatcgaaaaagctgaaatccgttaaagagttgctgggtattacgattatggaacgttcgagcttcgagaaaaatccgatcgacttcctggaggccaaaggctacaaggaagttaaaaaagacctgatcattaaactgccgaagtacagcctgttcgagctcgagaacggtcgcaaacgcatgctggcgagcgcgggtgagctccaaaaaggcaacgaattggctctgccgagcaagtacgtgaacttcctgtatctggcctcccattatgaaaaactgaagggtagcccggaagataacgaacaaaagcagctgttcgtggaacaacataaacactatctggacgagattattgagcaaatcagcgagttcagcaaacgcgtgattctggcggatgctaatctggacaaggttctttcagcctataataaacaccgtgataagccgatccgtgaacaggcagaaaatattattcacctgttcaccctgacgaacctgggtgcgccagcagcgtttaaatacttcgacacaacgatcgaccggaagcgttataccagcaccaaagaggttcttgacgccaccttgattcaccaaagcatcacgggtctgtatgaaacccgtattgacttgagccagttgggcggagattcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 23 - DNA, 3522 bp, synthetic construct
  source 1..3522: mol_type = other DNA
atgggaagtcatcaccaccaccaccattcaggtgagaacctgtacttccagagcatggctccgaaaaagaaacgtaaggtgggtatccacggcgttccggcgatgtcaagtaaaacaggacccgttgctgtagatccgacgctgagacgtcgcatcgagccgcacgaattcgaggtgttttttgatccgcgtgaactgcgcaaggagacgtgcctgctgtatgagatcaactggggtggccgtcacagcatttggcgtcacacctcccaaaacacgaacaaacacgtggaagtcaatttcatcgaaaaattcaccaccgaacgttacttctgcccgaacacccgttgttccatcacctggttcctgtcttggagcccgtgtggtgagtgcagccgcgcaattactgagttcctgagccgttacccgaatgttaccctgtttatctatatcgcccgtttgtaccatctggcgaacccgcgcaaccgtcagggtttacgcgacttaattagctctggcgttaccattcagattatgaccgagcaagagagcggttattgctggcataactttgtaaactatagcccgagcaatgaaagccattggcctcgctacccgcacctgtgggttcgtttgtacgtgctggaactgtactgcatcatccttggtttgccaccgtgcttgaatattttgagacgtaagcagtcgcaactgaccagttttacgatcgcgctacagagctgtcattatcagcgtctcccgccacacattctgtgggctaccggcctgaaaagcggcggtagcagcggtggctccagcggctcggaaactccgggcacctccgagtcagcgaccccggaatctggaagtgagacacccgggacttcagaatccgcgaccccggagtctggaatgccaaaacccgctgtagagtcagaattttcaaaagttttgaaaaagcactttccgggagaacgtttccgctcgagctatatgaaaagaggcggtaagatcctggctgcccaaggcgaagaggcagttgttgcgtatctacagggcaagtccgaggaggagcctccgaacttccagccaccggcgaagtgccacgtggtgaccaaatcccgtgacttcgcggaatggccgatcatgaaggcgtcggaagcgattcaacgttatatctatgcactgtcgaccaccgaacgcgcggcctgtaaaccgggcaaaagtagtgagagccatgcagcgtggttcgccgctaccggtgtcagcaaccacggctacagccacgttcagggtctgaacctgatttttgatcataccttgggccgttacgatggtgttctgaaaaaggtccaactgcgcaacgaaaaggcgagagcgcgtttagagagcattaacgcgtctagagcagatgaaggtttaccggagattaaagccgaggaggaagaagtggctacgaacgaaaccggccacctgttgcaaccgccgggtatcaatccgagcttttacgtgtatcagaccattagcccgcaggcataccgtccgcgtgacgagatcgtgctgccgccggagtacgcaggttacgtacgtgacccgaatgctccgattccgctgggtgtggtgcgtaaccgttgcgatatccaaaaagggtgcccgggctacatcccggaatggcagcgtgaagcaggtaccgcgatcagcccgaaaacaggcaaggccgtaactgtgccgggcctgtctccgaagaaaaacaaacgtatgcgtaggtactggcgctctgagaaggagaaagctcaagatgcgctgttggttaccgtacgcattggtaccgattgggttgtcatcgacgtgcgcggtctgttgcgcaacgcccgttggcgcaccatcgctcccaaggacatcagcctgaatgcgctcctcgatttgtttaccggtgatccggtgatcgatgtgcgtcgcaatattgttacctttacgtacaccctcgacgcctgtggtacctacgcccgtaaatggaccttgaaaggcaaacagacgaaagcgaccctggacaaactgacggccactcagacggttgcgttggttgcgattgcgcttggtcagaccaacccgatttccgctggcattagccgtgttacacaagaaaatggtgcactgcaatgcgaaccgctggaccggttcaccctgccggatgacctgttgaaggacatcagcgcatatcgcattgcttgggacagaaacgaagaggaactacgcgctcgcagcgtcgaggccctgccggaggcgcaacaagcggaggtccgtgcgctggacggggtgtccaaagaaaccgcgcgtacccaactgtgcgcagatttcggcctggacccgaaacgtctgccgtgggataaaatgagctccaatactacctttatcagcgaagccttgctgtctaacagcgtgagccgtgatcaggtttttttcactccggctccgaagaagggtgctaagaaaaaagcgcctgttgaagttatgcgtaaagaccgcacttgggcacgtgcgtacaagccgcgtctgagcgtcgaagctcagaaactgaagaacgaagccctgtgggcacttaagcgcacctccccggagtatttgaagctgagccgtcgcaaagaggagctgtgtcgtcgtagcatcaactatgttatcgaaaagacccgtcgccgtacgcaatgtcagattgtgatcccggtgatcgaggatctgaatgttcgcttctttcatggttctggtaagagactgcccggttgggataatttcttcaccgctaaaaaagagaaccgttggttcatccagggcctgcacaaagcgtttagcgatctgcgtacgcaccgttccttctatgtttttgaagttcgtccggagcgcacctctattacctgtccaaagtgcggtcattgcgaggtgggtaatcgtgacggtgaggcgtttcagtgcctgagctgcggcaagacgtgcaacgcagatcttgacgtcgcgacccataatctgacccaagtggcgttgaccggtaagaccatgccgaagcgcgaggagccgcgtgacgctcagggtacagcgccagcgcgcaagactaagaaggcgagcaaatcaaaggcgccaccggcggagcgcgaagaccagaccccggcgcaagagccaagccagaccagttctggaggtagcgggggtagcggtggtagcactaacttaagtgatattatcgagaaagaaaccgggaaacagctggtgatccaagaaagcattctgatgctgcccgaagaagttgaagaagtgattggtaacaagcctgagtcggacatcttagtgcacaccgcgtacgatgaaagcacggatgagaatgtgatgctgctcacctctgatgcccccgaatacaagccgtgggctctggtgatccaggactctaacggagaaaacaaaattaagatgctctctggcggctccatggcgcctaaaaaaaaacgcaaagtggggattcacggggttccggcgtaa

SEQ ID NO: 24 - DNA, 4938 bp, synthetic construct
  source 1..4938: mol_type = other DNA
atgggaagtcatcaccaccaccaccattcaggtgagaacctgtacttccagagcatggctccgaaaaagaaacgtaaggtgggtatccacggcgttccggcgatgtcaagtaaaacaggacccgttgctgtagatccgacgctgagacgtcgcatcgagccgcacgaattcgaggtgttttttgatccgcgtgaactgcgcaaggagacgtgcctgctgtatgagatcaactggggtggccgtcacagcatttggcgtcacacctcccaaaacacgaacaaacacgtggaagtcaatttcatcgaaaaattcaccaccgaacgttacttctgcccgaacacccgttgttccatcacctggttcctgtcttggagcccgtgtggtgagtgcagccgcgcaattactgagttcctgagccgttacccgaatgttaccctgtttatctatatcgcccgtttgtaccatctggcgaacccgcgcaaccgtcagggtttacgcgacttaattagctctggcgttaccattcagattatgaccgagcaagagagcggttattgctggcataactttgtaaactatagcccgagcaatgaaagccattggcctcgctacccgcacctgtgggttcgtttgtacgtgctggaactgtactgcatcatccttggtttgccaccgtgcttgaatattttgagacgtaagcagtcgcaactgaccagttttacgatcgcgctacagagctgtcattatcagcgtctcccgccacacattctgtgggctaccggcctgaaaagcggcggtagcagcggtggctccagcggctcggaaactccgggcacctccgagtcagcgaccccggaatctggaagtgagacacccgggacttcagaatccgcgaccccggagtctggaatgtcaaaactagaaaagtttacaaattgttactccctgtcgaaaaccctgagattcaaagcgatcccggtgggtaaaactcaagagaacattgataacaaacgtctgctggttgaggacgaaaagcgcgcggaggattacaaaggcgttaaaaagctgttggaccgttattacctgtcctttattaatgatgttctgcacagcattaaattaaagaacctaaacaattacatctccctcttccgtaaaaagacccgcaccgaaaaggaaaataaagaactggagaaccttgagattaatctgcgtaaagaaatcgctaaagcgtttaaaggcaacgagggttataaatccttatttaaaaaagacatcatcgaaacgattctcccagaattcctggacgacaaagacgagatcgcgctggttaattccttcaacggctttaccaccgcattcaccggcttttttgataaccgcgaaaacatgttctctgaagaggccaaaagcacctcaattgcgtttcgctgcattaacgaaaatctgacgcgttatatctctaacatggatatcttcgagaaggttgacgctatctttgataaacatgaagtccaggagatcaaggagaagatcctgaacagcgactacgacgtggaggatttcttcgagggtgagttcttcaattttgttttgacccaagaaggcatcgatgtctacaatgcgataattggtggttttgttaccgagagcggggagaaaattaagggtctgaacgaatatatcaacttgtacaaccagaaaaccaaacaaaaactgccgaagtttaagccgctgtacaagcaagtcttgtcggatcgtgaatcgctgagcttttacggcgaaggctacaccagcgatgaggaggtgttggaggtgttccgcaatacgttgaataagaactctgaaattttttccagcattaagaagctggaaaaactattcaagaacttcgacgagtatagcagcgcaggtatctttgtgaaaaacggtccggctattagcaccatctcgaaagacattttcggcgaatggaatgttattcgtgataagtggaatgccgaatacgacgacattcatctgaagaaaaaggcggttgtaactgaaaagtacgaggacgatcgtcgtaagtcgttcaaaaagatcggctccttctctctggagcaactgcaagagtacgccgatgccgatttgtcagtcgtggagaagctgaaagagatcatcattcaaaaagttgacgaaatctataaggtctatggtagcagtgaaaaactgtttgacgcggacttcgtgctggagaaaagcctgaagaaaaatgacgccgtggtggcaatcatgaaagatcttctggatagcgtcaagtctttcgaaaactacatcaaagcgtttttcggtgaaggcaaggaaacgaatcgtgacgagagcttctatggtgatttcgtcctagcctatgatatcctgcttaaggtggatcacatctatgatgccatccgcaactatgttacccagaaaccgtactcaaaggacaagttcaagttatacttccagaatccgcagtttatgggtggctgggataaagataaggaaaccgactatcgcgccaccattttacgttacggtagtaagtactatctggcaatcatggataaaaaatatgcgaaatgcctgcaaaagatcgacaaggacgatgttaacggcaattatgagaagatcaactacaaactgctgccgggcccaaataagatgctgccgaaggtgtttttcagcaaaaaatggatggcgtattacaatccgagcgaggacatccaaaagatctacaaaaacggtacgtttaagaagggtgatatgttcaacctcaacgattgtcataaacttattgacttcttcaaggatagcatatctcgttatccgaagtggagcaacgcgtatgactttaactttagcgaaaccgagaaatacaaagatatcgcgggtttctatcgtgaagttgaagaacagggttacaaagtgtccttcgagtccgcgagcaagaaggaggtagacaagctggtcgaggagggtaagttgtacatgtttcagatttacaataaggacttttccgacaagtcccatggtacaccgaatttgcacaccatgtatttcaagcttctgttcgacgagaacaaccacggccagatccgtctgagcggtggtgcagagctgttcatgcgtagagctagcctgaagaaagaagagttggtcgttcatccggctaattcgccgatcgcgaacaaaaatccagataaccctaaaaagactacgaccctgagctatgatgtgtataaagacaaacgctttagcgaagaccagtatgagttacacattccgatcgctatcaacaagtgcccgaagaatattttcaagatcaacaccgaagtgcgtgttcttttgaagcacgatgacaacccgtatgtgattggtattgctcgtggtgaacgtaatctgctgtacattgtggttgtggacggcaaaggaaacatcgtggaacagtacagcctgaacgaaatcattaacaatttcaacggcattcgcattaagacggattaccattctttgctggacaaaaaggagaaagagcgtttcgaggcccgtcagaactggacctccatcgagaacattaaggagctgaaggcgggttacatcagccaggtggtgcacaaaatctgcgaactggtggagaagtacgacgcggtgattgctttggcggatttgaactccggttttaagaacagccgcgttaaagtggaaaagcaggtttatcagaaattcgaaaaaatgctgattgataaattgaattacatggttgacaaaaagtctaacccgtgtgcaaccggcggtgcgctgaagggctaccaaatcaccaataagtttgaaagctttaagagcatgagcacgcaaaatggcttcattttttacattccggcgtggctgaccagcaagatcgatccgtcaaccggctttgttaatttactgaagacgaagtatacctctattgccgactctaaaaaatttattagttccttcgatagaatcatgtatgttccggaagaggacctgttcgaatttgcactggactacaagaactttagccgtaccgatgctgattatataaagaaatggaaactctacagctatggcaaccgcatccgtatctttcgcaacccgaaaaaaaacaacgtgttcgactgggaagaggtatgtctgacttcagcgtacaaggagcttttcaataaatatggtatcaactaccagcagggtgatatccgtgcactgctttgcgagcaaagcgataaagcgttctattcaagcttcatggcgctgatgagcctgatgctgcaaatgcgcaacagcatcaccggtcgtaccgatgttgcatttctgatttctccagtaaaaaacagcgatggtatcttttacgacagccgtaattacgaggcccaagaaaatgcgattttgccgaagaacgctgacgcgaatggtgcatacaacattgcaaggaaagttctgtgggcaatcggccaatttaaaaaagcggaagatgagaagctggacaaagtgaaaattgcgattagcaacaaggagtggctggagtatgcgcagacctccgttaaatcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 25 - DNA, 5301 bp, synthetic construct
  source 1..5301: mol_type = other DNA
atgggaagtcatcaccaccaccaccattcaggtgagaacctgtacttccagagcatggctccgaaaaagaaacgtaaggtgggtatccacggcgttccggcgatgtcaacagatgctgaatatgtaaggatacacgagaagctggacatctacacctttaaaaagcagttctccaacaataaaaaatctgtcagccatcgttgctacgtgttgtttgaactcaagcgtcgtggcgaacgccgtgcgtgcttttggggttacgctgtgaacaaaccgcagagcggtacggaacgtggtattcatgcggagatcttcagcatccgcaaggtggaagagtatctgcgcgataacccgggtcaattcaccattaactggtacagcagctggtcaccgtgcgccgactgtgctgagaagattttggagtggtataaccaagaattgcgcggtaatggccatactctgaagatctgggtgtgtaaactttattacgaaaagaacgcgcgtaatcaaattggtctgtggaatctgagagataacggcgttggtctgaatgttatggtttccgagcactatcagtgctgccgtaaaatcttcattcagtccagccacaaccaactgaacgagaaccgttggctggaaaagaccctgaagcgcgcggagaaacgtcgtagcgagctgagcattatgtttcaggttaaaatcttacacaccaccaaatcgccggcagtctctggcggttcctcgggcggcagctctggctccgaaaccccaggtacgagcgagagcgcaaccccggaatctggaagtgagacacccgggacttcagaatccgcgaccccggagtctggaatggataaaaagtattcaataggactagctattggcaccaacagcgttggttgggcagttatcaccgatgaatataaggtcccgagcaagaagtttaaggtcctgggcaacaccgatcgccacagcataaaaaagaaccttattggtgcgctgctgttcgattctggtgagactgccgaagcgacccgtctgaagagaaccgcgcgtcgtagatacacccggcgcaagaaccgtatctgctatctgcaagaaattttcagcaacgagatggctaaggtcgacgactccttctttcatcgcctggaggaaagctttctggttgaggaagacaaaaagcacgagcgtcatccgattttcggtaacattgttgatgaagttgcctaccacgaaaaataccccacaatttatcatctgcgtaaaaaactggtagactccaccgataaggcggatttacgccttatctatttggcgctggcgcatatgattaagttccgcggtcattttttgatcgagggcgacctgaatccggataacagcgacgttgacaaactgtttatccagctggtgcagacctataaccagttgttcgaagagaacccgattaatgcgtctggcgtggacgcgaaggcgatcctgagcgctcgtctgtccaagtcacgccgcctggagaacttgatcgcgcaactgccgggtgagaagaaaaacggcttgttcggcaatctgattgctctgtccctaggtttgaccccgaatttcaaatctaattttgatctggcggaagatgcgaaattacagttgagcaaggacacttacgatgacgatctggacaacctgctcgctcagatcggcgatcagtatgccgatcttttcctggccgcgaagaacctgagcgatgccatcttgctatcggacatcttacgtgttaacaccgaaatcaccaaagctccgctaagcgcgtccatgattaaacgttacgatgaacaccaccaggatctgacgctgctgaaagcgttggtgcgccagcaactgccggaaaaatacaaggaaattttcttcgaccaaagcaagaacggttatgcaggctatatcgacggtggtgcgtcgcaagaagaattttataaattcatcaaaccgattttggagaagatggatggtaccgaggagctgctggtaaaattgaatcgggaagacctcttgcgaaagcagcgtactttcgataatggtagcatcccgcaccagattcatctgggcgaacttcacgcaatcctacgtcgtcaagaggatttctacccgtttctgaaggataaccgtgaaaagatcgagaagattctgacctttcgtattccgtattatgtgggtccgctggcgagaggcaacagccgttttgcgtggatgacccgcaagtcggaagagactatcacgccgtggaatttcgaggaggtcgtggacaagggtgcatcagcgcaatcttttattgaacgtatgaccaattttgataaaaaccttccaaatgaaaaagtgttgccgaagcacagcttgctgtacgagtatttcaccgtttacaatgaactgacgaaagtcaagtacgttaccgaaggtatgcgtaagccagcctttctgtctggggagcaaaaaaaagccattgtggatctgctgttcaagaccaatcgcaaggttaccgtcaagcaactgaaagaggattacttcaaaaaaatcgaatgttttgactccgtggaaatcagcggcgttgaagaccgctttaatgcatctctgggcacctaccacgatctactcaagatcatcaaggataaggacttcctggacaacgaagaaaatgaggatatcttggaagatattgtgctgactttaacgttgttcgaggatcgtgagatgattgaagaacgcctcaaaacgtacgcacatctgtttgacgataaggtgatgaaacagctaaagcgccgtcgttacaccggttggggtcgtctgtctagaaagctgattaacggcattcgtgacaaacaatctggcaagaccatcctggacttcctcaaatccgacggcttcgcaaatcgtaatttcatgcaactgatccatgatgatagcctgacctttaaagaagatattcagaaggctcaagtgtccggccagggtgactcgctgcatgagcatatcgcgaatctggctggctcccctgcgataaaaaaaggtattctccaaacggtgaaagttgtggatgaactggttaaagtcatgggtcgtcataaaccggaaaacatcgtgattgagatggcacgtgagaaccagaccacccagaaaggtcaaaaaaacagtcgtgagcgtatgaaacgtatcgaagaggggattaaggagttaggcagccagattctgaaggagcacccggttgagaacacccagctgcaaaacgagaagctctacctgtattacctgcaaaacggccgtgacatgtatgttgaccaggagctggacatcaatcgtctcagcgactacgacgtggatgctatcgtgccgcagagctttctaaaagatgattccatcgacaataaagtgctcacccgtagcgacaagaaccgtggtaaaagcgacaacgtgccgagcgaggaagtagttaagaaaatgaaaaactactggcgtcagctattgaacgccaagcttatcacccagcgtaagtttgacaacttgacgaaggcagagcgtggcggcctgagcgagctagacaaggcgggttttattaaacgccagctggtggaaactcgccagattaccaaacacgttgcgcaaatcttggactcacgcatgaatacgaagtacgacgaaaacgataaattgatccgcgaggtgaaggtcataaccctgaaatctaaactggttagcgatttccggaaggactttcaattttataaagtccgggagattaacaactatcaccatgcacacgacgcttacttaaatgcggttgttggcacggccctgatcaagaagtacccgaaactggaaagcgagtttgtttatggtgactacaaggtgtatgacgttcgcaagatgattgcaaaatccgaacaagagattggtaaggcgacggctaagtattttttctacagcaatatcatgaacttcttcaagactgaaatcaccctggcgaacggtgaaattcgtaaacgtccgttgatcgaaaccaacggtgaaaccggtgaaatcgtttgggataaaggtagggattttgcgaccgttcgtaaggtcctgagcatgccgcaggttaacatcgtgaaaaagaccgaagtgcagaccggcggtttctccaaggagagtatcttaccgaagcgcaacagtgacaagctgatcgcgcgtaaaaaagactgggatccgaaaaaatacggcggctttgactccccgaccgtcgcgtacagcgtactggtcgtggctaaagtggagaaaggcaaatcgaaaaagctgaaatccgttaaagagttgctgggtattacgattatggaacgttcgagcttcgagaaaaatccgatcgacttcctggaggccaaaggctacaaggaagttaaaaaagacctgatcattaaactgccgaagtacagcctgttcgagctcgagaacggtcgcaaacgcatgctggcgagcgcgggtgagctccaaaaaggcaacgaattggctctgccgagcaagtacgtgaacttcctgtatctggcctcccattatgaaaaactgaagggtagcccggaagataacgaacaaaagcagctgttcgtggaacaacataaacactatctggacgagattattgagcaaatcagcgagttcagcaaacgcgtgattctggcggatgctaatctggacaaggttctttcagcctataataaacaccgtgataagccgatccgtgaacaggcagaaaatattattcacctgttcaccctgacgaacctgggtgcgccagcagcgtttaaatacttcgacacaacgatcgaccggaagcgttataccagcaccaaagaggttcttgacgccaccttgattcaccaaagcatcacgggtctgtatgaaacccgtattgacttgagccagttgggcggagattcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 26 - DNA, 3462 bp, synthetic construct
  source 1..3462: mol_type = other DNA
atgggaagtcatcaccaccaccaccattcaggtgagaacctgtacttccagagcatggctccgaaaaagaaacgtaaggtgggtatccacggcgttccggcgatgtcaacagatgctgaatatgtaaggatacacgagaagctggacatctacacctttaaaaagcagttctccaacaataaaaaatctgtcagccatcgttgctacgtgttgtttgaactcaagcgtcgtggcgaacgccgtgcgtgcttttggggttacgctgtgaacaaaccgcagagcggtacggaacgtggtattcatgcggagatcttcagcatccgcaaggtggaagagtatctgcgcgataacccgggtcaattcaccattaactggtacagcagctggtcaccgtgcgccgactgtgctgagaagattttggagtggtataaccaagaattgcgcggtaatggccatactctgaagatctgggtgtgtaaactttattacgaaaagaacgcgcgtaatcaaattggtctgtggaatctgagagataacggcgttggtctgaatgttatggtttccgagcactatcagtgctgccgtaaaatcttcattcagtccagccacaaccaactgaacgagaaccgttggctggaaaagaccctgaagcgcgcggagaaacgtcgtagcgagctgagcattatgtttcaggttaaaatcttacacaccaccaaatcgccggcagtctctggcggttcctcgggcggcagctctggctccgaaaccccaggtacgagcgagagcgcaaccccggaatctggaagtgagacacccgggacttcagaatccgcgaccccggagtctggaatgccaaaacccgctgtagagtcagaattttcaaaagttttgaaaaagcactttccgggagaacgtttccgctcgagctatatgaaaagaggcggtaagatcctggctgcccaaggcgaagaggcagttgttgcgtatctacagggcaagtccgaggaggagcctccgaacttccagccaccggcgaagtgccacgtggtgaccaaatcccgtgacttcgcggaatggccgatcatgaaggcgtcggaagcgattcaacgttatatctatgcactgtcgaccaccgaacgcgcggcctgtaaaccgggcaaaagtagtgagagccatgcagcgtggttcgccgctaccggtgtcagcaaccacggctacagccacgttcagggtctgaacctgatttttgatcataccttgggccgttacgatggtgttctgaaaaaggtccaactgcgcaacgaaaaggcgagagcgcgtttagagagcattaacgcgtctagagcagatgaaggtttaccggagattaaagccgaggaggaagaagtggctacgaacgaaaccggccacctgttgcaaccgccgggtatcaatccgagcttttacgtgtatcagaccattagcccgcaggcataccgtccgcgtgacgagatcgtgctgccgccggagtacgcaggttacgtacgtgacccgaatgctccgattccgctgggtgtggtgcgtaaccgttgcgatatccaaaaagggtgcccgggctacatcccggaatggcagcgtgaagcaggtaccgcgatcagcccgaaaacaggcaaggccgtaactgtgccgggcctgtctccgaagaaaaacaaacgtatgcgtaggtactggcgctctgagaaggagaaagctcaagatgcgctgttggttaccgtacgcattggtaccgattgggttgtcatcgacgtgcgcggtctgttgcgcaacgcccgttggcgcaccatcgctcccaaggacatcagcctgaatgcgctcctcgatttgtttaccggtgatccggtgatcgatgtgcgtcgcaatattgttacctttacgtacaccctcgacgcctgtggtacctacgcccgtaaatggaccttgaaaggcaaacagacgaaagcgaccctggacaaactgacggccactcagacggttgcgttggttgcgattgcgcttggtcagaccaacccgatttccgctggcattagccgtgttacacaagaaaatggtgcactgcaatgcgaaccgctggaccggttcaccctgccggatgacctgttgaaggacatcagcgcatatcgcattgcttgggacagaaacgaagaggaactacgcgctcgcagcgtcgaggccctgccggaggcgcaacaagcggaggtccgtgcgctggacggggtgtccaaagaaaccgcgcgtacccaactgtgcgcagatttcggcctggacccgaaacgtctgccgtgggataaaatgagctccaatactacctttatcagcgaagccttgctgtctaacagcgtgagccgtgatcaggtttttttcactccggctccgaagaagggtgctaagaaaaaagcgcctgttgaagttatgcgtaaagaccgcacttgggcacgtgcgtacaagccgcgtctgagcgtcgaagctcagaaactgaagaacgaagccctgtgggcacttaagcgcacctccccggagtatttgaagctgagccgtcgcaaagaggagctgtgtcgtcgtagcatcaactatgttatcgaaaagacccgtcgccgtacgcaatgtcagattgtgatcccggtgatcgaggatctgaatgttcgcttctttcatggttctggtaagagactgcccggttgggataatttcttcaccgctaaaaaagagaaccgttggttcatccagggcctgcacaaagcgtttagcgatctgcgtacgcaccgttccttctatgtttttgaagttcgtccggagcgcacctctattacctgtccaaagtgcggtcattgcgaggtgggtaatcgtgacggtgaggcgtttcagtgcctgagctgcggcaagacgtgcaacgcagatcttgacgtcgcgacccataatctgacccaagtggcgttgaccggtaagaccatgccgaagcgcgaggagccgcgtgacgctcagggtacagcgccagcgcgcaagactaagaaggcgagcaaatcaaaggcgccaccggcggagcgcgaagaccagaccccggcgcaagagccaagccagaccagttctggaggtagcgggggtagcggtggtagcactaacttaagtgatattatcgagaaagaaaccgggaaacagctggtgatccaagaaagcattctgatgctgcccgaagaagttgaagaagtgattggtaacaagcctgagtcggacatcttagtgcacaccgcgtacgatgaaagcacggatgagaatgtgatgctgctcacctctgatgcccccgaatacaagccgtgggctctggtgatccaggactctaacggagaaaacaaaattaagatgctctctggcggctccatggcgcctaaaaaaaaacgcaaagtggggattcacggggttccggcgtaa

SEQ ID NO: 27 - DNA, 4878 bp, synthetic construct
  source 1..4878: mol_type = other DNA
atgggaagtcatcaccaccaccaccattcaggtgagaacctgtacttccagagcatggctccgaaaaagaaacgtaaggtgggtatccacggcgttccggcgatgtcaacagatgctgaatatgtaaggatacacgagaagctggacatctacacctttaaaaagcagttctccaacaataaaaaatctgtcagccatcgttgctacgtgttgtttgaactcaagcgtcgtggcgaacgccgtgcgtgcttttggggttacgctgtgaacaaaccgcagagcggtacggaacgtggtattcatgcggagatcttcagcatccgcaaggtggaagagtatctgcgcgataacccgggtcaattcaccattaactggtacagcagctggtcaccgtgcgccgactgtgctgagaagattttggagtggtataaccaagaattgcgcggtaatggccatactctgaagatctgggtgtgtaaactttattacgaaaagaacgcgcgtaatcaaattggtctgtggaatctgagagataacggcgttggtctgaatgttatggtttccgagcactatcagtgctgccgtaaaatcttcattcagtccagccacaaccaactgaacgagaaccgttggctggaaaagaccctgaagcgcgcggagaaacgtcgtagcgagctgagcattatgtttcaggttaaaatcttacacaccaccaaatcgccggcagtctctggcggttcctcgggcggcagctctggctccgaaaccccaggtacgagcgagagcgcaaccccggaatctggaagtgagacacccgggacttcagaatccgcgaccccggagtctggaatgtcaaaactagaaaagtttacaaattgttactccctgtcgaaaaccctgagattcaaagcgatcccggtgggtaaaactcaagagaacattgataacaaacgtctgctggttgaggacgaaaagcgcgcggaggattacaaaggcgttaaaaagctgttggaccgttattacctgtcctttattaatgatgttctgcacagcattaaattaaagaacctaaacaattacatctccctcttccgtaaaaagacccgcaccgaaaaggaaaataaagaactggagaaccttgagattaatctgcgtaaagaaatcgctaaagcgtttaaaggcaacgagggttataaatccttatttaaaaaagacatcatcgaaacgattctcccagaattcctggacgacaaagacgagatcgcgctggttaattccttcaacggctttaccaccgcattcaccggcttttttgataaccgcgaaaacatgttctctgaagaggccaaaagcacctcaattgcgtttcgctgcattaacgaaaatctgacgcgttatatctctaacatggatatcttcgagaaggttgacgctatctttgataaacatgaagtccaggagatcaaggagaagatcctgaacagcgactacgacgtggaggatttcttcgagggtgagttcttcaattttgttttgacccaagaaggcatcgatgtctacaatgcgataattggtggttttgttaccgagagcggggagaaaattaagggtctgaacgaatatatcaacttgtacaaccagaaaaccaaacaaaaactgccgaagtttaagccgctgtacaagcaagtcttgtcggatcgtgaatcgctgagcttttacggcgaaggctacaccagcgatgaggaggtgttggaggtgttccgcaatacgttgaataagaactctgaaattttttccagcattaagaagctggaaaaactattcaagaacttcgacgagtatagcagcgcaggtatctttgtgaaaaacggtccggctattagcaccatctcgaaagacattttcggcgaatggaatgttattcgtgataagtggaatgccgaatacgacgacattcatctgaagaaaaaggcggttgtaactgaaaagtacgaggacgatcgtcgtaagtcgttcaaaaagatcggctccttctctctggagcaactgcaagagtacgccgatgccgatttgtcagtcgtggagaagctgaaagagatcatcattcaaaaagttgacgaaatctataaggtctatggtagcagtgaaaaactgtttgacgcggacttcgtgctggagaaaagcctgaagaaaaatgacgccgtggtggcaatcatgaaagatcttctggatagcgtcaagtctttcgaaaactacatcaaagcgtttttcggtgaaggcaaggaaacgaatcgtgacgagagcttctatggtgatttcgtcctagcctatgatatcctgcttaaggtggatcacatctatgatgccatccgcaactatgttacccagaaaccgtactcaaaggacaagttcaagttatacttccagaatccgcagtttatgggtggctgggataaagataaggaaaccgactatcgcgccaccattttacgttacggtagtaagtactatctggcaatcatggataaaaaatatgcgaaatgcctgcaaaagatcgacaaggacgatgttaacggcaattatgagaagatcaactacaaactgctgccgggcccaaataagatgctgccgaaggtgtttttcagcaaaaaatggatggcgtattacaatccgagcgaggacatccaaaagatctacaaaaacggtacgtttaagaagggtgatatgttcaacctcaacgattgtcataaacttattgacttcttcaaggatagcatatctcgttatccgaagtggagcaacgcgtatgactttaactttagcgaaaccgagaaatacaaagatatcgcgggtttctatcgtgaagttgaagaacagggttacaaagtgtccttcgagtccgcgagcaagaaggaggtagacaagctggtcgaggagggtaagttgtacatgtttcagatttacaataaggacttttccgacaagtcccatggtacaccgaatttgcacaccatgtatttcaagcttctgttcgacgagaacaaccacggccagatccgtctgagcggtggtgcagagctgttcatgcgtagagctagcctgaagaaagaagagttggtcgttcatccggctaattcgccgatcgcgaacaaaaatccagataaccctaaaaagactacgaccctgagctatgatgtgtataaagacaaacgctttagcgaagaccagtatgagttacacattccgatcgctatcaacaagtgcccgaagaatattttcaagatcaacaccgaagtgcgtgttcttttgaagcacgatgacaacccgtatgtgattggtattgctcgtggtgaacgtaatctgctgtacattgtggttgtggacggcaaaggaaacatcgtggaacagtacagcctgaacgaaatcattaacaatttcaacggcattcgcattaagacggattaccattctttgctggacaaaaaggagaaagagcgtttcgaggcccgtcagaactggacctccatcgagaacattaaggagctgaaggcgggttacatcagccaggtggtgcacaaaatctgcgaactggtggagaagtacgacgcggtgattgctttggcggatttgaactccggttttaagaacagccgcgttaaagtggaaaagcaggtttatcagaaattcgaaaaaatgctgattgataaattgaattacatggttgacaaaaagtctaacccgtgtgcaaccggcggtgcgctgaagggctaccaaatcaccaataagtttgaaagctttaagagcatgagcacgcaaaatggcttcattttttacattccggcgtggctgaccagcaagatcgatccgtcaaccggctttgttaatttactgaagacgaagtatacctctattgccgactctaaaaaatttattagttccttcgatagaatcatgtatgttccggaagaggacctgttcgaatttgcactggactacaagaactttagccgtaccgatgctgattatataaagaaatggaaactctacagctatggcaaccgcatccgtatctttcgcaacccgaaaaaaaacaacgtgttcgactgggaagaggtatgtctgacttcagcgtacaaggagcttttcaataaatatggtatcaactaccagcagggtgatatccgtgcactgctttgcgagcaaagcgataaagcgttctattcaagcttcatggcgctgatgagcctgatgctgcaaatgcgcaacagcatcaccggtcgtaccgatgttgcatttctgatttctccagtaaaaaacagcgatggtatcttttacgacagccgtaattacgaggcccaagaaaatgcgattttgccgaagaacgctgacgcgaatggtgcatacaacattgcaaggaaagttctgtgggcaatcggccaatttaaaaaagcggaagatgagaagctggacaaagtgaaaattgcgattagcaacaaggagtggctggagtatgcgcagacctccgttaaatcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 28 - DNA, 5163 bp, synthetic construct
  source 1..5163: mol_type = other DNA
atgggaagtcatcaccaccaccaccattcaggtgagaacctgtacttccagagcatggctccgaaaaagaaacgtaaggtgggtatccacggcgttccggcgatgtcatttgagaggaattatgatcccagagaattgcgcaaagagacgtacctgctgtatgaaatcaagtggggtaaaagcggcaagctgtggcgtcattggtgtcaaaacaaccgcacccaacacgcggaagtttatttcctcgagaatattttcaacgcccgtcgttttaacccgagcacccactgctccatcacctggtatctttcttggtcgccgtgtgcggagtgctcgcaaaaaatcgtggacttcctgaaagaacatccgaatgtgaacctggagatctatgttgcgagattgtactatccggagaacgaacgtaatcgtcagggcctgcgcgacttggtcaacagcggggtgaccattcgtattatggatctgccggattacaattactgctggaaaacctttgtttccgatcagggtggcgacgaagactactggcctggtcactttgctccgtggattaagcagtacagcctgaagctgagcggtggttcttccggcggtagcagtggctccgagactccaggtacgagcgagagcgcaaccccggaatctggaagtgagacacccgggacttcagaatccgcgaccccggagtctggaatggataaaaagtattcaataggactagctattggcaccaacagcgttggttgggcagttatcaccgatgaatataaggtcccgagcaagaagtttaaggtcctgggcaacaccgatcgccacagcataaaaaagaaccttattggtgcgctgctgttcgattctggtgagactgccgaagcgacccgtctgaagagaaccgcgcgtcgtagatacacccggcgcaagaaccgtatctgctatctgcaagaaattttcagcaacgagatggctaaggtcgacgactccttctttcatcgcctggaggaaagctttctggttgaggaagacaaaaagcacgagcgtcatccgattttcggtaacattgttgatgaagttgcctaccacgaaaaataccccacaatttatcatctgcgtaaaaaactggtagactccaccgataaggcggatttacgccttatctatttggcgctggcgcatatgattaagttccgcggtcattttttgatcgagggcgacctgaatccggataacagcgacgttgacaaactgtttatccagctggtgcagacctataaccagttgttcgaagagaacccgattaatgcgtctggcgtggacgcgaaggcgatcctgagcgctcgtctgtccaagtcacgccgcctggagaacttgatcgcgcaactgccgggtgagaagaaaaacggcttgttcggcaatctgattgctctgtccctaggtttgaccccgaatttcaaatctaattttgatctggcggaagatgcgaaattacagttgagcaaggacacttacgatgacgatctggacaacctgctcgctcagatcggcgatcagtatgccgatcttttcctggccgcgaagaacctgagcgatgccatcttgctatcggacatcttacgtgttaacaccgaaatcaccaaagctccgctaagcgcgtccatgattaaacgttacgatgaacaccaccaggatctgacgctgctgaaagcgttggtgcgccagcaactgccggaaaaatacaaggaaattttcttcgaccaaagcaagaacggttatgcaggctatatcgacggtggtgcgtcgcaagaagaattttataaattcatcaaaccgattttggagaagatggatggtaccgaggagctgctggtaaaattgaatcgggaagacctcttgcgaaagcagcgtactttcgataatggtagcatcccgcaccagattcatctgggcgaacttcacgcaatcctacgtcgtcaagaggatttctacccgtttctgaaggataaccgtgaaaagatcgagaagattctgacctttcgtattccgtattatgtgggtccgctggcgagaggcaacagccgttttgcgtggatgacccgcaagtcggaagagactatcacgccgtggaatttcgaggaggtcgtggacaagggtgcatcagcgcaatcttttattgaacgtatgaccaattttgataaaaaccttccaaatgaaaaagtgttgccgaagcacagcttgctgtacgagtatttcaccgtttacaatgaactgacgaaagtcaagtacgttaccgaaggtatgcgtaagccagcctttctgtctggggagcaaaaaaaagccattgtggatctgctgttcaagaccaatcgcaaggttaccgtcaagcaactgaaagaggattacttcaaaaaaatcgaatgttttgactccgtggaaatcagcggcgttgaagaccgctttaatgcatctctgggcacctaccacgatctactcaagatcatcaaggataaggacttcctggacaacgaagaaaatgaggatatcttggaagatattgtgctgactttaacgttgttcgaggatcgtgagatgattgaagaacgcctcaaaacgtacgcacatctgtttgacgataaggtgatgaaacagctaaagcgccgtcgttacaccggttggggtcgtctgtctagaaagctgattaacggcattcgtgacaaacaatctggcaagaccatcctggacttcctcaaatccgacggcttcgcaaatcgtaatttcatgcaactgatccatgatgatagcctgacctttaaagaagatattcagaaggctcaagtgtccggccagggtgactcgctgcatgagcatatcgcgaatctggctggctcccctgcgataaaaaaaggtattctccaaacggtgaaagttgtggatgaactggttaaagtcatgggtcgtcataaaccggaaaacatcgtgattgagatggcacgtgagaaccagaccacccagaaaggtcaaaaaaacagtcgtgagcgtatgaaacgtatcgaagaggggattaaggagttaggcagccagattctgaaggagcacccggttgagaacacccagctgcaaaacgagaagctctacctgtattacctgcaaaacggccgtgacatgtatgttgaccaggagctggacatcaatcgtctcagcgactacgacgtggatgctatcgtgccgcagagctttctaaaagatgattccatcgacaataaagtgctcacccgtagcgacaagaaccgtggtaaaagcgacaacgtgccgagcgaggaagtagttaagaaaatgaaaaactactggcgtcagctattgaacgccaagcttatcacccagcgtaagtttgacaacttgacgaaggcagagcgtggcggcctgagcgagctagacaaggcgggttttattaaacgccagctggtggaaactcgccagattaccaaacacgttgcgcaaatcttggactcacgcatgaatacgaagtacgacgaaaacgataaattgatccgcgaggtgaaggtcataaccctgaaatctaaactggttagcgatttccggaaggactttcaattttataaagtccgggagattaacaactatcaccatgcacacgacgcttacttaaatgcggttgttggcacggccctgatcaagaagtacccgaaactggaaagcgagtttgtttatggtgactacaaggtgtatgacgttcgcaagatgattgcaaaatccgaacaagagattggtaaggcgacggctaagtattttttctacagcaatatcatgaacttcttcaagactgaaatcaccctggcgaacggtgaaattcgtaaacgtccgttgatcgaaaccaacggtgaaaccggtgaaatcgtttgggataaaggtagggattttgcgaccgttcgtaaggtcctgagcatgccgcaggttaacatcgtgaaaaagaccgaagtgcagaccggcggtttctccaaggagagtatcttaccgaagcgcaacagtgacaagctgatcgcgcgtaaaaaagactgggatccgaaaaaatacggcggctttgactccccgaccgtcgcgtacagcgtactggtcgtggctaaagtggagaaaggcaaatcgaaaaagctgaaatccgttaaagagttgctgggtattacgattatggaacgttcgagcttcgagaaaaatccgatcgacttcctggaggccaaaggctacaaggaagttaaaaaagacctgatcattaaactgccgaagtacagcctgttcgagctcgagaacggtcgcaaacgcatgctggcgagcgcgggtgagctccaaaaaggcaacgaattggctctgccgagcaagtacgtgaacttcctgtatctggcctcccattatgaaaaactgaagggtagcccggaagataacgaacaaaagcagctgttcgtggaacaacataaacactatctggacgagattattgagcaaatcagcgagttcagcaaacgcgtgattctggcggatgctaatctggacaaggttctttcagcctataataaacaccgtgataagccgatccgtgaacaggcagaaaatattattcacctgttcaccctgacgaacctgggtgcgccagcagcgtttaaatacttcgacacaacgatcgaccggaagcgttataccagcaccaaagaggttcttgacgccaccttgattcaccaaagcatcacgggtctgtatgaaacccgtattgacttgagccagttgggcggagattcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 29 - DNA, 3324 bp, synthetic construct
  source 1..3324: mol_type = other DNA
atgggaagtcatcaccaccaccaccattcaggtgagaacctgtacttccagagcatggctccgaaaaagaaacgtaaggtgggtatccacggcgttccggcgatgtcatttgagaggaattatgatcccagagaattgcgcaaagagacgtacctgctgtatgaaatcaagtggggtaaaagcggcaagctgtggcgtcattggtgtcaaaacaaccgcacccaacacgcggaagtttatttcctcgagaatattttcaacgcccgtcgttttaacccgagcacccactgctccatcacctggtatctttcttggtcgccgtgtgcggagtgctcgcaaaaaatcgtggacttcctgaaagaacatccgaatgtgaacctggagatctatgttgcgagattgtactatccggagaacgaacgtaatcgtcagggcctgcgcgacttggtcaacagcggggtgaccattcgtattatggatctgccggattacaattactgctggaaaacctttgtttccgatcagggtggcgacgaagactactggcctggtcactttgctccgtggattaagcagtacagcctgaagctgagcggtggttcttccggcggtagcagtggctccgagactccaggtacgagcgagagcgcaaccccggaatctggaagtgagacacccgggacttcagaatccgcgaccccggagtctggaatgccaaaacccgctgtagagtcagaattttcaaaagttttgaaaaagcactttccgggagaacgtttccgctcgagctatatgaaaagaggcggtaagatcctggctgcccaaggcgaagaggcagttgttgcgtatctacagggcaagtccgaggaggagcctccgaacttccagccaccggcgaagtgccacgtggtgaccaaatcccgtgacttcgcggaatggccgatcatgaaggcgtcggaagcgattcaacgttatatctatgcactgtcgaccaccgaacgcgcggcctgtaaaccgggcaaaagtagtgagagccatgcagcgtggttcgccgctaccggtgtcagcaaccacggctacagccacgttcagggtctgaacctgatttttgatcataccttgggccgttacgatggtgttctgaaaaaggtccaactgcgcaacgaaaaggcgagagcgcgtttagagagcattaacgcgtctagagcagatgaaggtttaccggagattaaagccgaggaggaagaagtggctacgaacgaaaccggccacctgttgcaaccgccgggtatcaatccgagcttttacgtgtatcagaccattagcccgcaggcataccgtccgcgtgacgagatcgtgctgccgccggagtacgcaggttacgtacgtgacccgaatgctccgattccgctgggtgtggtgcgtaaccgttgcgatatccaaaaagggtgcccgggctacatcccggaatggcagcgtgaagcaggtaccgcgatcagcccgaaaacaggcaaggccgtaactgtgccgggcctgtctccgaagaaaaacaaacgtatgcgtaggtactggcgctctgagaaggagaaagctcaagatgcgctgttggttaccgtacgcattggtaccgattgggttgtcatcgacgtgcgcggtctgttgcgcaacgcccgttggcgcaccatcgctcccaaggacatcagcctgaatgcgctcctcgatttgtttaccggtgatccggtgatcgatgtgcgtcgcaatattgttacctttacgtacaccctcgacgcctgtggtacctacgcccgtaaatggaccttgaaaggcaaacagacgaaagcgaccctggacaaactgacggccactcagacggttgcgttggttgcgattgcgcttggtcagaccaacccgatttccgctggcattagccgtgttacacaagaaaatggtgcactgcaatgcgaaccgctggaccggttcaccctgccggatgacctgttgaaggacatcagcgcatatcgcattgcttgggacagaaacgaagaggaactacgcgctcgcagcgtcgaggccctgccggaggcgcaacaagcggaggtccgtgcgctggacggggtgtccaaagaaaccgcgcgtacccaactgtgcgcagatttcggcctggacccgaaacgtctgccgtgggataaaatgagctccaatactacctttatcagcgaagccttgctgtctaacagcgtgagccgtgatcaggtttttttcactccggctccgaagaagggtgctaagaaaaaagcgcctgttgaagttatgcgtaaagaccgcacttgggcacgtgcgtacaagccgcgtctgagcgtcgaagctcagaaactgaagaacgaagccctgtgggcacttaagcgcacctccccggagtatttgaagctgagccgtcgcaaagaggagctgtgtcgtcgtagcatcaactatgttatcgaaaagacccgtcgccgtacgcaatgtcagattgtgatcccggtgatcgaggatctgaatgttcgcttctttcatggttctggtaagagactgcccggttgggataatttcttcaccgctaaaaaagagaaccgttggttcatccagggcctgcacaaagcgtttagcgatctgcgtacgcaccgttccttctatgtttttgaagttcgtccggagcgcacctctattacctgtccaaagtgcggtcattgcgaggtgggtaatcgtgacggtgaggcgtttcagtgcctgagctgcggcaagacgtgcaacgcagatcttgacgtcgcgacccataatctgacccaagtggcgttgaccggtaagaccatgccgaagcgcgaggagccgcgtgacgctcagggtacagcgccagcgcgcaagactaagaaggcgagcaaatcaaaggcgccaccggcggagcgcgaagaccagaccccggcgcaagagccaagccagaccagttctggaggtagcgggggtagcggtggtagcactaacttaagtgatattatcgagaaagaaaccgggaaacagctggtgatccaagaaagcattctgatgctgcccgaagaagttgaagaagtgattggtaacaagcctgagtcggacatcttagtgcacaccgcgtacgatgaaagcacggatgagaatgtgatgctgctcacctctgatgcccccgaatacaagccgtgggctctggtgatccaggactctaacggagaaaacaaaattaagatgctctctggcggctccatggcgcctaaaaaaaaacgcaaagtggggattcacggggttccggcgtaa

SEQ ID NO: 30 - DNA, 4740 bp, synthetic construct
  source 1..4740: mol_type = other DNA
atgggaagtcatcaccaccaccaccattcaggtgagaacctgtacttccagagcatggctccgaaaaagaaacgtaaggtgggtatccacggcgttccggcgatgtcatttgagaggaattatgatcccagagaattgcgcaaagagacgtacctgctgtatgaaatcaagtggggtaaaagcggcaagctgtggcgtcattggtgtcaaaacaaccgcacccaacacgcggaagtttatttcctcgagaatattttcaacgcccgtcgttttaacccgagcacccactgctccatcacctggtatctttcttggtcgccgtgtgcggagtgctcgcaaaaaatcgtggacttcctgaaagaacatccgaatgtgaacctggagatctatgttgcgagattgtactatccggagaacgaacgtaatcgtcagggcctgcgcgacttggtcaacagcggggtgaccattcgtattatggatctgccggattacaattactgctggaaaacctttgtttccgatcagggtggcgacgaagactactggcctggtcactttgctccgtggattaagcagtacagcctgaagctgagcggtggttcttccggcggtagcagtggctccgagactccaggtacgagcgagagcgcaaccccggaatctggaagtgagacacccgggacttcagaatccgcgaccccggagtctggaatgtcaaaactagaaaagtttacaaattgttactccctgtcgaaaaccctgagattcaaagcgatcccggtgggtaaaactcaagagaacattgataacaaacgtctgctggttgaggacgaaaagcgcgcggaggattacaaaggcgttaaaaagctgttggaccgttattacctgtcctttattaatgatgttctgcacagcattaaattaaagaacctaaacaattacatctccctcttccgtaaaaagacccgcaccgaaaaggaaaataaagaactggagaaccttgagattaatctgcgtaaagaaatcgctaaagcgtttaaaggcaacgagggttataaatccttatttaaaaaagacatcatcgaaacgattctcccagaattcctggacgacaaagacgagatcgcgctggttaattccttcaacggctttaccaccgcattcaccggcttttttgataaccgcgaaaacatgttctctgaagaggccaaaagcacctcaattgcgtttcgctgcattaacgaaaatctgacgcgttatatctctaacatggatatcttcgagaaggttgacgctatctttgataaacatgaagtccaggagatcaaggagaagatcctgaacagcgactacgacgtggaggatttcttcgagggtgagttcttcaattttgttttgacccaagaaggcatcgatgtctacaatgcgataattggtggttttgttaccgagagcggggagaaaattaagggtctgaacgaatatatcaacttgtacaaccagaaaaccaaacaaaaactgccgaagtttaagccgctgtacaagcaagtcttgtcggatcgtgaatcgctgagcttttacggcgaaggctacaccagcgatgaggaggtgttggaggtgttccgcaatacgttgaataagaactctgaaattttttccagcattaagaagctggaaaaactattcaagaacttcgacgagtatagcagcgcaggtatctttgtgaaaaacggtccggctattagcaccatctcgaaagacattttcggcgaatggaatgttattcgtgataagtggaatgccgaatacgacgacattcatctgaagaaaaaggcggttgtaactgaaaagtacgaggacgatcgtcgtaagtcgttcaaaaagatcggctccttctctctggagcaactgcaagagtacgccgatgccgatttgtcagtcgtggagaagctgaaagagatcatcattcaaaaagttgacgaaatctataaggtctatggtagcagtgaaaaactgtttgacgcggacttcgtgctggagaaaagcctgaagaaaaatgacgccgtggtggcaatcatgaaagatcttctggatagcgtcaagtctttcgaaaactacatcaaagcgtttttcggtgaaggcaaggaaacgaatcgtgacgagagcttctatggtgatttcgtcctagcctatgatatcctgcttaaggtggatcacatctatgatgccatccgcaactatgttacccagaaaccgtactcaaaggacaagttcaagttatacttccagaatccgcagtttatgggtggctgggataaagataaggaaaccgactatcgcgccaccattttacgttacggtagtaagtactatctggcaatcatggataaaaaatatgcgaaatgcctgcaaaagatcgacaaggacgatgttaacggcaattatgagaagatcaactacaaactgctgccgggcccaaataagatgctgccgaaggtgtttttcagcaaaaaatggatggcgtattacaatccgagcgaggacatccaaaagatctacaaaaacggtacgtttaagaagggtgatatgttcaacctcaacgattgtcataaacttattgacttcttcaaggatagcatatctcgttatccgaagtggagcaacgcgtatgactttaactttagcgaaaccgagaaatacaaagatatcgcgggtttctatcgtgaagttgaagaacagggttacaaagtgtccttcgagtccgcgagcaagaaggaggtagacaagctggtcgaggagggtaagttgtacatgtttcagatttacaataaggacttttccgacaagtcccatggtacaccgaatttgcacaccatgtatttcaagcttctgttcgacgagaacaaccacggccagatccgtctgagcggtggtgcagagctgttcatgcgtagagctagcctgaagaaagaagagttggtcgttcatccggctaattcgccgatcgcgaacaaaaatccagataaccctaaaaagactacgaccctgagctatgatgtgtataaagacaaacgctttagcgaagaccagtatgagttacacattccgatcgctatcaacaagtgcccgaagaatattttcaagatcaacaccgaagtgcgtgttcttttgaagcacgatgacaacccgtatgtgattggtattgctcgtggtgaacgtaatctgctgtacattgtggttgtggacggcaaaggaaacatcgtggaacagtacagcctgaacgaaatcattaacaatttcaacggcattcgcattaagacggattaccattctttgctggacaaaaaggagaaagagcgtttcgaggcccgtcagaactggacctccatcgagaacattaaggagctgaaggcgggttacatcagccaggtggtgcacaaaatctgcgaactggtggagaagtacgacgcggtgattgctttggcggatttgaactccggttttaagaacagccgcgttaaagtggaaaagcaggtttatcagaaattcgaaaaaatgctgattgataaattgaattacatggttgacaaaaagtctaacccgtgtgcaaccggcggtgcgctgaagggctaccaaatcaccaataagtttgaaagctttaagagcatgagcacgcaaaatggcttcattttttacattccggcgtggctgaccagcaagatcgatccgtcaaccggctttgttaatttactgaagacgaagtatacctctattgccgactctaaaaaatttattagttccttcgatagaatcatgtatgttccggaagaggacctgttcgaatttgcactggactacaagaactttagccgtaccgatgctgattatataaagaaatggaaactctacagctatggcaaccgcatccgtatctttcgcaacccgaaaaaaaacaacgtgttcgactgggaagaggtatgtctgacttcagcgtacaaggagcttttcaataaatatggtatcaactaccagcagggtgatatccgtgcactgctttgcgagcaaagcgataaagcgttctattcaagcttcatggcgctgatgagcctgatgctgcaaatgcgcaacagcatcaccggtcgtaccgatgttgcatttctgatttctccagtaaaaaacagcgatggtatcttttacgacagccgtaattacgaggcccaagaaaatgcgattttgccgaagaacgctgacgcgaatggtgcatacaacattgcaaggaaagttctgtgggcaatcggccaatttaaaaaagcggaagatgagaagctggacaaagtgaaaattgcgattagcaacaaggagtggctggagtatgcgcagacctccgttaaatcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 31 - DNA, 5169 bp, synthetic construct
  source 1..5169: mol_type = other DNA
atgggaagtcatcaccaccaccaccattcaggtgagaacctgtacttccagagcatggctccgaaaaagaaacgtaaggtgggtatccacggcgttccggcgatggatcccccaacatttactttcaactttaataacgaaccgtgggttcgcggtcgccacgaaacctacctgtgctatgaggtggaacgtatgcataatgatacttgggttctgctgaatcaacgtagaggctttttatgcaaccaagctccgcataaacatggttttctggaaggccgtcacgccgagctgtgcttccttgacgtgatcccattctggaaactggacttggaccaggattatcgtgtgacctgtttcaccagctggtcgccgtgcttcagctgtgcgcaggagatggcgaagttcatctccaaaaacaagcacgttagcttgtgcatttttaccgcgcgtatttacgatgatcaaggtcgctgccaagagggcctgcgtaccctggcagaggctggtgcgaagatctctatcatgacgtacagcgaatttaaacactgctgggacacgtttgtcgatcaccagggttgtccgttccagccgtgggacggcttggacgaacatagccaggatttgtccggtcgtctgcgcgcaattctacagaatcaagagaactctggaagtgagacacccgggacttcagaatccgcgaccccggagtctggaatggataaaaagtattcaataggactagctattggcaccaacagcgttggttgggcagttatcaccgatgaatataaggtcccgagcaagaagtttaaggtcctgggcaacaccgatcgccacagcataaaaaagaaccttattggtgcgctgctgttcgattctggtgagactgccgaagcgacccgtctgaagagaaccgcgcgtcgtagatacacccggcgcaagaaccgtatctgctatctgcaagaaattttcagcaacgagatggctaaggtcgacgactccttctttcatcgcctggaggaaagctttctggttgaggaagacaaaaagcacgagcgtcatccgattttcggtaacattgttgatgaagttgcctaccacgaaaaataccccacaatttatcatctgcgtaaaaaactggtagactccaccgataaggcggatttacgccttatctatttggcgctggcgcatatgattaagttccgcggtcattttttgatcgagggcgacctgaatccggataacagcgacgttgacaaactgtttatccagctggtgcagacctataaccagttgttcgaagagaacccgattaatgcgtctggcgtggacgcgaaggcgatcctgagcgctcgtctgtccaagtcacgccgcctggagaacttgatcgcgcaactgccgggtgagaagaaaaacggcttgttcggcaatctgattgctctgtccctaggtttgaccccgaatttcaaatctaattttgatctggcggaagatgcgaaattacagttgagcaaggacacttacgatgacgatctggacaacctgctcgctcagatcggcgatcagtatgccgatcttttcctggccgcgaagaacctgagcgatgccatcttgctatcggacatcttacgtgttaacaccgaaatcaccaaagctccgctaagcgcgtccatgattaaacgttacgatgaacaccaccaggatctgacgctgctgaaagcgttggtgcgccagcaactgccggaaaaatacaaggaaattttcttcgaccaaagcaagaacggttatgcaggctatatcgacggtggtgcgtcgcaagaagaattttataaattcatcaaaccgattttggagaagatggatggtaccgaggagctgctggtaaaattgaatcgggaagacctcttgcgaaagcagcgtactttcgataatggtagcatcccgcaccagattcatctgggcgaacttcacgcaatcctacgtcgtcaagaggatttctacccgtttctgaaggataaccgtgaaaagatcgagaagattctgacctttcgtattccgtattatgtgggtccgctggcgagaggcaacagccgttttgcgtggatgacccgcaagtcggaagagactatcacgccgtggaatttcgaggaggtcgtggacaagggtgcatcagcgcaatcttttattgaacgtatgaccaattttgataaaaaccttccaaatgaaaaagtgttgccgaagcacagcttgctgtacgagtatttcaccgtttacaatgaactgacgaaagtcaagtacgttaccgaaggtatgcgtaagccagcctttctgtctggggagcaaaaaaaagccattgtggatctgctgttcaagaccaatcgcaaggttaccgtcaagcaactgaaagaggattacttcaaaaaaatcgaatgttttgactccgtggaaatcagcggcgttgaagaccgctttaatgcatctctgggcacctaccacgatctactcaagatcatcaaggataaggacttcctggacaacgaagaaaatgaggatatcttggaagatattgtgctgactttaacgttgttcgaggatcgtgagatgattgaagaacgcctcaaaacgtacgcacatctgtttgacgataaggtgatgaaacagctaaagcgccgtcgttacaccggttggggtcgtctgtctagaaagctgattaacggcattcgtgacaaacaatctggcaagaccatcctggacttcctcaaatccgacggcttcgcaaatcgtaatttcatgcaactgatccatgatgatagcctgacctttaaagaagatattcagaaggctcaagtgtccggccagggtgactcgctgcatgagcatatcgcgaatctggctggctcccctgcgataaaaaaaggtattctccaaacggtgaaagttgtggatgaactggttaaagtcatgggtcgtcataaaccggaaaacatcgtgattgagatggcacgtgagaaccagaccacccagaaaggtcaaaaaaacagtcgtgagcgtatgaaacgtatcgaagaggggattaaggagttaggcagccagattctgaaggagcacccggttgagaacacccagctgcaaaacgagaagctctacctgtattacctgcaaaacggccgtgacatgtatgttgaccaggagctggacatcaatcgtctcagcgactacgacgtggatgctatcgtgccgcagagctttctaaaagatgattccatcgacaataaagtgctcacccgtagcgacaagaaccgtggtaaaagcgacaacgtgccgagcgaggaagtagttaagaaaatgaaaaactactggcgtcagctattgaacgccaagcttatcacccagcgtaagtttgacaacttgacgaaggcagagcgtggcggcctgagcgagctagacaaggcgggttttattaaacgccagctggtggaaactcgccagattaccaaacacgttgcgcaaatcttggactcacgcatgaatacgaagtacgacgaaaacgataaattgatccgcgaggtgaaggtcataaccctgaaatctaaactggttagcgatttccggaaggactttcaattttataaagtccgggagattaacaactatcaccatgcacacgacgcttacttaaatgcggttgttggcacggccctgatcaagaagtacccgaaactggaaagcgagtttgtttatggtgactacaaggtgtatgacgttcgcaagatgattgcaaaatccgaacaagagattggtaaggcgacggctaagtattttttctacagcaatatcatgaacttcttcaagactgaaatcaccctggcgaacggtgaaattcgtaaacgtccgttgatcgaaaccaacggtgaaaccggtgaaatcgtttgggataaaggtagggattttgcgaccgttcgtaaggtcctgagcatgccgcaggttaacatcgtgaaaaagaccgaagtgcagaccggcggtttctccaaggagagtatcttaccgaagcgcaacagtgacaagctgatcgcgcgtaaaaaagactgggatccgaaaaaatacggcggctttgactccccgaccgtcgcgtacagcgtactggtcgtggctaaagtggagaaaggcaaatcgaaaaagctgaaatccgttaaagagttgctgggtattacgattatggaacgttcgagcttcgagaaaaatccgatcgacttcctggaggccaaaggctacaaggaagttaaaaaagacctgatcattaaactgccgaagtacagcctgttcgagctcgagaacggtcgcaaacgcatgctggcgagcgcgggtgagctccaaaaaggcaacgaattggctctgccgagcaagtacgtgaacttcctgtatctggcctcccattatgaaaaactgaagggtagcccggaagataacgaacaaaagcagctgttcgtggaacaacataaacactatctggacgagattattgagcaaatcagcgagttcagcaaacgcgtgattctggcggatgctaatctggacaaggttctttcagcctataataaacaccgtgataagccgatccgtgaacaggcagaaaatattattcacctgttcaccctgacgaacctgggtgcgccagcagcgtttaaatacttcgacacaacgatcgaccggaagcgttataccagcaccaaagaggttcttgacgccaccttgattcaccaaagcatcacgggtctgtatgaaacccgtattgacttgagccagttgggcggagattcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 32 - DNA, 3330 bp, synthetic construct
  source 1..3330: mol_type = other DNA
atgggaagtcatcaccaccaccaccattcaggtgagaacctgtacttccagagcatggctccgaaaaagaaacgtaaggtgggtatccacggcgttccggcgatggatcccccaacatttactttcaactttaataacgaaccgtgggttcgcggtcgccacgaaacctacctgtgctatgaggtggaacgtatgcataatgatacttgggttctgctgaatcaacgtagaggctttttatgcaaccaagctccgcataaacatggttttctggaaggccgtcacgccgagctgtgcttccttgacgtgatcccattctggaaactggacttggaccaggattatcgtgtgacctgtttcaccagctggtcgccgtgcttcagctgtgcgcaggagatggcgaagttcatctccaaaaacaagcacgttagcttgtgcatttttaccgcgcgtatttacgatgatcaaggtcgctgccaagagggcctgcgtaccctggcagaggctggtgcgaagatctctatcatgacgtacagcgaatttaaacactgctgggacacgtttgtcgatcaccagggttgtccgttccagccgtgggacggcttggacgaacatagccaggatttgtccggtcgtctgcgcgcaattctacagaatcaagagaactctggaagtgagacacccgggacttcagaatccgcgaccccggagtctggaatgccaaaacccgctgtagagtcagaattttcaaaagttttgaaaaagcactttccgggagaacgtttccgctcgagctatatgaaaagaggcggtaagatcctggctgcccaaggcgaagaggcagttgttgcgtatctacagggcaagtccgaggaggagcctccgaacttccagccaccggcgaagtgccacgtggtgaccaaatcccgtgacttcgcggaatggccgatcatgaaggcgtcggaagcgattcaacgttatatctatgcactgtcgaccaccgaacgcgcggcctgtaaaccgggcaaaagtagtgagagccatgcagcgtggttcgccgctaccggtgtcagcaaccacggctacagccacgttcagggtctgaacctgatttttgatcataccttgggccgttacgatggtgttctgaaaaaggtccaactgcgcaacgaaaaggcgagagcgcgtttagagagcattaacgcgtctagagcagatgaaggtttaccggagattaaagccgaggaggaagaagtggctacgaacgaaaccggccacctgttgcaaccgccgggtatcaatccgagcttttacgtgtatcagaccattagcccgcaggcataccgtccgcgtgacgagatcgtgctgccgccggagtacgcaggttacgtacgtgacccgaatgctccgattccgctgggtgtggtgcgtaaccgttgcgatatccaaaaagggtgcccgggctacatcccggaatggcagcgtgaagcaggtaccgcgatcagcccgaaaacaggcaaggccgtaactgtgccgggcctgtctccgaagaaaaacaaacgtatgcgtaggtactggcgctctgagaaggagaaagctcaagatgcgctgttggttaccgtacgcattggtaccgattgggttgtcatcgacgtgcgcggtctgttgcgcaacgcccgttggcgcaccatcgctcccaaggacatcagcctgaatgcgctcctcgatttgtttaccggtgatccggtgatcgatgtgcgtcgcaatattgttacctttacgtacaccctcgacgcctgtggtacctacgcccgtaaatggaccttgaaaggcaaacagacgaaagcgaccctggacaaactgacggccactcagacggttgcgttggttgcgattgcgcttggtcagaccaacccgatttccgctggcattagccgtgttacacaagaaaatggtgcactgcaatgcgaaccgctggaccggttcaccctgccggatgacctgttgaaggacatcagcgcatatcgcattgcttgggacagaaacgaagaggaactacgcgctcgcagcgtcgaggccctgccggaggcgcaacaagcggaggtccgtgcgctggacggggtgtccaaagaaaccgcgcgtacccaactgtgcgcagatttcggcctggacccgaaacgtctgccgtgggataaaatgagctccaatactacctttatcagcgaagccttgctgtctaacagcgtgagccgtgatcaggtttttttcactccggctccgaagaagggtgctaagaaaaaagcgcctgttgaagttatgcgtaaagaccgcacttgggcacgtgcgtacaagccgcgtctgagcgtcgaagctcagaaactgaagaacgaagccctgtgggcacttaagcgcacctccccggagtatttgaagctgagccgtcgcaaagaggagctgtgtcgtcgtagcatcaactatgttatcgaaaagacccgtcgccgtacgcaatgtcagattgtgatcccggtgatcgaggatctgaatgttcgcttctttcatggttctggtaagagactgcccggttgggataatttcttcaccgctaaaaaagagaaccgttggttcatccagggcctgcacaaagcgtttagcgatctgcgtacgcaccgttccttctatgtttttgaagttcgtccggagcgcacctctattacctgtccaaagtgcggtcattgcgaggtgggtaatcgtgacggtgaggcgtttcagtgcctgagctgcggcaagacgtgcaacgcagatcttgacgtcgcgacccataatctgacccaagtggcgttgaccggtaagaccatgccgaagcgcgaggagccgcgtgacgctcagggtacagcgccagcgcgcaagactaagaaggcgagcaaatcaaaggcgccaccggcggagcgcgaagaccagaccccggcgcaagagccaagccagaccagttctggaggtagcgggggtagcggtggtagcactaacttaagtgatattatcgagaaagaaaccgggaaacagctggtgatccaagaaagcattctgatgctgcccgaagaagttgaagaagtgattggtaacaagcctgagtcggacatcttagtgcacaccgcgtacgatgaaagcacggatgagaatgtgatgctgctcacctctgatgcccccgaatacaagccgtgggctctggtgatccaggactctaacggagaaaacaaaattaagatgctctctggcggctccatggcgcctaaaaaaaaacgcaaagtggggattcacggggttccggcgtaa

SEQ ID NO: 33 - DNA, 4746 bp, synthetic construct
  source 1..4746: mol_type = other DNA
atgggaagtcatcaccaccaccaccattcaggtgagaacctgtacttccagagcatggctccgaaaaagaaacgtaaggtgggtatccacggcgttccggcgatggatcccccaacatttactttcaactttaataacgaaccgtgggttcgcggtcgccacgaaacctacctgtgctatgaggtggaacgtatgcataatgatacttgggttctgctgaatcaacgtagaggctttttatgcaaccaagctccgcataaacatggttttctggaaggccgtcacgccgagctgtgcttccttgacgtgatcccattctggaaactggacttggaccaggattatcgtgtgacctgtttcaccagctggtcgccgtgcttcagctgtgcgcaggagatggcgaagttcatctccaaaaacaagcacgttagcttgtgcatttttaccgcgcgtatttacgatgatcaaggtcgctgccaagagggcctgcgtaccctggcagaggctggtgcgaagatctctatcatgacgtacagcgaatttaaacactgctgggacacgtttgtcgatcaccagggttgtccgttccagccgtgggacggcttggacgaacatagccaggatttgtccggtcgtctgcgcgcaattctacagaatcaagagaactctggaagtgagacacccgggacttcagaatccgcgaccccggagtctggaatgtcaaaactagaaaagtttacaaattgttactccctgtcgaaaaccctgagattcaaagcgatcccggtgggtaaaactcaagagaacattgataacaaacgtctgctggttgaggacgaaaagcgcgcggaggattacaaaggcgttaaaaagctgttggaccgttattacctgtcctttattaatgatgttctgcacagcattaaattaaagaacctaaacaattacatctccctcttccgtaaaaagacccgcaccgaaaaggaaaataaagaactggagaaccttgagattaatctgcgtaaagaaatcgctaaagcgtttaaaggcaacgagggttataaatccttatttaaaaaagacatcatcgaaacgattctcccagaattcctggacgacaaagacgagatcgcgctggttaattccttcaacggctttaccaccgcattcaccggcttttttgataaccgcgaaaacatgttctctgaagaggccaaaagcacctcaattgcgtttcgctgcattaacgaaaatctgacgcgttatatctctaacatggatatcttcgagaaggttgacgctatctttgataaacatgaagtccaggagatcaaggagaagatcctgaacagcgactacgacgtggaggatttcttcgagggtgagttcttcaattttgttttgacccaagaaggcatcgatgtctacaatgcgataattggtggttttgttaccgagagcggggagaaaattaagggtctgaacgaatatatcaacttgtacaaccagaaaaccaaacaaaaactgccgaagtttaagccgctgtacaagcaagtcttgtcggatcgtgaatcgctgagcttttacggcgaaggctacaccagcgatgaggaggtgttggaggtgttccgcaatacgttgaataagaactctgaaattttttccagcattaagaagctggaaaaactattcaagaacttcgacgagtatagcagcgcaggtatctttgtgaaaaacggtccggctattagcaccatctcgaaagacattttcggcgaatggaatgttattcgtgataagtggaatgccgaatacgacgacattcatctgaagaaaaaggcggttgtaactgaaaagtacgaggacgatcgtcgtaagtcgttcaaaaagatcggctccttctctctggagcaactgcaagagtacgccgatgccgatttgtcagtcgtggagaagctgaaagagatcatcattcaaaaagttgacgaaatctataaggtctatggtagcagtgaaaaactgtttgacgcggacttcgtgctggagaaaagcctgaagaaaaatgacgccgtggtggcaatcatgaaagatcttctggatagcgtcaagtctttcgaaaactacatcaaagcgtttttcggtgaaggcaaggaaacgaatcgtgacgagagcttctatggtgatttcgtcctagcctatgatatcctgcttaaggtggatcacatctatgatgccatccgcaactatgttacccagaaaccgtactcaaaggacaagttcaagttatacttccagaatccgcagtttatgggtggctgggataaagataaggaaaccgactatcgcgccaccattttacgttacggtagtaagtactatctggcaatcatggataaaaaatatgcgaaatgcctgcaaaagatcgacaaggacgatgttaacggcaattatgagaagatcaactacaaactgctgccgggcccaaataagatgctgccgaaggtgtttttcagcaaaaaatggatggcgtattacaatccgagcgaggacatccaaaagatctacaaaaacggtacgtttaagaagggtgatatgttcaacctcaacgattgtcataaacttattgacttcttcaaggatagcatatctcgttatccgaagtggagcaacgcgtatgactttaactttagcgaaaccgagaaatacaaagatatcgcgggtttctatcgtgaagttgaagaacagggttacaaagtgtccttcgagtccgcgagcaagaaggaggtagacaagctggtcgaggagggtaagttgtacatgtttcagatttacaataaggacttttccgacaagtcccatggtacaccgaatttgcacaccatgtatttcaagcttctgttcgacgagaacaaccacggccagatccgtctgagcggtggtgcagagctgttcatgcgtagagctagcctgaagaaagaagagttggtcgttcatccggctaattcgccgatcgcgaacaaaaatccagataaccctaaaaagactacgaccctgagctatgatgtgtataaagacaaacgctttagcgaagaccagtatgagttacacattccgatcgctatcaacaagtgcccgaagaatattttcaagatcaacaccgaagtgcgtgttcttttgaagcacgatgacaacccgtatgtgattggtattgctcgtggtgaacgtaatctgctgtacattgtggttgtggacggcaaaggaaacatcgtggaacagtacagcctgaacgaaatcattaacaatttcaacggcattcgcattaagacggattaccattctttgctggacaaaaaggagaaagagcgtttcgaggcccgtcagaactggacctccatcgagaacattaaggagctgaaggcgggttacatcagccaggtggtgcacaaaatctgcgaactggtggagaagtacgacgcggtgattgctttggcggatttgaactccggttttaagaacagccgcgttaaagtggaaaagcaggtttatcagaaattcgaaaaaatgctgattgataaattgaattacatggttgacaaaaagtctaacccgtgtgcaaccggcggtgcgctgaagggctaccaaatcaccaataagtttgaaagctttaagagcatgagcacgcaaaatggcttcattttttacattccggcgtggctgaccagcaagatcgatccgtcaaccggctttgttaatttactgaagacgaagtatacctctattgccgactctaaaaaatttattagttccttcgatagaatcatgtatgttccggaagaggacctgttcgaatttgcactggactacaagaactttagccgtaccgatgctgattatataaagaaatggaaactctacagctatggcaaccgcatccgtatctttcgcaacccgaaaaaaaacaacgtgttcgactgggaagaggtatgtctgacttcagcgtacaaggagcttttcaataaatatggtatcaactaccagcagggtgatatccgtgcactgctttgcgagcaaagcgataaagcgttctattcaagcttcatggcgctgatgagcctgatgctgcaaatgcgcaacagcatcaccggtcgtaccgatgttgcatttctgatttctccagtaaaaaacagcgatggtatcttttacgacagccgtaattacgaggcccaagaaaatgcgattttgccgaagaacgctgacgcgaatggtgcatacaacattgcaaggaaagttctgtgggcaatcggccaatttaaaaaagcggaagatgagaagctggacaaagtgaaaattgcgattagcaacaaggagtggctggagtatgcgcagacctccgttaaatcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 34 - DNA, 4560 bp, synthetic construct
  source 1..4560: mol_type = other DNA
atgggcagccaccaccaccaccaccacagcggcgagaacctgtacttccagtccatggctccaaagaagaagagaaaggttggaatccacggagttccagcttctggaatggataaaaagtattcaataggactagctattggcaccaacagcgttggttgggcagttatcaccgatgaatataaggtcccgagcaagaagtttaaggtcctgggcaacaccgatcgccacagcataaaaaagaaccttattggtgcgctgctgttcgattctggtgagactgccgaagcgacccgtctgaagagaaccgcgcgtcgtagatacacccggcgcaagaaccgtatctgctatctgcaagaaattttcagcaacgagatggctaaggtcgacgactccttctttcatcgcctggaggaaagctttctggttgaggaagacaaaaagcacgagcgtcatccgattttcggtaacattgttgatgaagttgcctaccacgaaaaataccccacaatttatcatctgcgtaaaaaactggtagactccaccgataaggcggatttacgccttatctatttggcgctggcgcatatgattaagttccgcggtcattttttgatcgagggcgacctgaatccggataacagcgacgttgacaaactgtttatccagctggtgcagacctataaccagttgttcgaagagaacccgattaatgcgtctggcgtggacgcgaaggcgatcctgagcgctcgtctgtccaagtcacgccgcctggagaacttgatcgcgcaactgccgggtgagaagaaaaacggcttgttcggcaatctgattgctctgtccctaggtttgaccccgaatttcaaatctaattttgatctggcggaagatgcgaaattacagttgagcaaggacacttacgatgacgatctggacaacctgctcgctcagatcggcgatcagtatgccgatcttttcctggccgcgaagaacctgagcgatgccatcttgctatcggacatcttacgtgttaacaccgaaatcaccaaagctccgctaagcgcgtccatgattaaacgttacgatgaacaccaccaggatctgacgctgctgaaagcgttggtgcgccagcaactgccggaaaaatacaaggaaattttcttcgaccaaagcaagaacggttatgcaggctatatcgacggtggtgcgtcgcaagaagaattttataaattcatcaaaccgattttggagaagatggatggtaccgaggagctgctggtaaaattgaatcgggaagacctcttgcgaaagcagcgtactttcgataatggtagcatcccgcaccagattcatctgggcgaacttcacgcaatcctacgtcgtcaagaggatttctacccgtttctgaaggataaccgtgaaaagatcgagaagattctgacctttcgtattccgtattatgtgggtccgctggcgagaggcaacagccgttttgcgtggatgacccgcaagtcggaagagactatcacgccgtggaatttcgaggaggtcgtggacaagggtgcatcagcgcaatcttttattgaacgtatgaccaattttgataaaaaccttccaaatgaaaaagtgttgccgaagcacagcttgctgtacgagtatttcaccgtttacaatgaactgacgaaagtcaagtacgttaccgaaggtatgcgtaagccagcctttctgtctggggagcaaaaaaaagccattgtggatctgctgttcaagaccaatcgcaaggttaccgtcaagcaactgaaagaggattacttcaaaaaaatcgaatgttttgactccgtggaaatcagcggcgttgaagaccgctttaatgcatctctgggcacctaccacgatctactcaagatcatcaaggataaggacttcctggacaacgaagaaaatgaggatatcttggaagatattgtgctgactttaacgttgttcgaggatcgtgagatgattgaagaacgcctcaaaacgtacgcacatctgtttgacgataaggtgatgaaacagctaaagcgccgtcgttacaccggttggggtcgtctgtctagaaagctgattaacggcattcgtgacaaacaatctggcaagaccatcctggacttcctcaaatccgacggcttcgcaaatcgtaatttcatgcaactgatccatgatgatagcctgacctttaaagaagatattcagaaggctcaagtgtccggccagggtgactcgctgcatgagcatatcgcgaatctggctggctcccctgcgataaaaaaaggtattctccaaacggtgaaagttgtggatgaactggttaaagtcatgggtcgtcataaaccggaaaacatcgtgattgagatggcacgtgagaaccagaccacccagaaaggtcaaaaaaacagtcgtgagcgtatgaaacgtatcgaagaggggattaaggagttaggcagccagattctgaaggagcacccggttgagaacacccagctgcaaaacgagaagctctacctgtattacctgcaaaacggccgtgacatgtatgttgaccaggagctggacatcaatcgtctcagcgactacgacgtggatgctatcgtgccgcagagctttctaaaagatgattccatcgacaataaagtgctcacccgtagcgacaagaaccgtggtaaaagcgacaacgtgccgagcgaggaagtagttaagaaaatgaaaaactactggcgtcagctattgaacgccaagcttatcacccagcgtaagtttgacaacttgacgaaggcagagcgtggcggcctgagcgagctagacaaggcgggttttattaaacgccagctggtggaaactcgccagattaccaaacacgttgcgcaaatcttggactcacgcatgaatacgaagtacgacgaaaacgataaattgatccgcgaggtgaaggtcataaccctgaaatctaaactggttagcgatttccggaaggactttcaattttataaagtccgggagattaacaactatcaccatgcacacgacgcttacttaaatgcggttgttggcacggccctgatcaagaagtacccgaaactggaaagcgagtttgtttatggtgactacaaggtgtatgacgttcgcaagatgattgcaaaatccgaacaagagattggtaaggcgacggctaagtattttttctacagcaatatcatgaacttcttcaagactgaaatcaccctggcgaacggtgaaattcgtaaacgtccgttgatcgaaaccaacggtgaaaccggtgaaatcgtttgggataaaggtagggattttgcgaccgttcgtaaggtcctgagcatgccgcaggttaacatcgtgaaaaagaccgaagtgcagaccggcggtttctccaaggagagtatcttaccgaagcgcaacagtgacaagctgatcgcgcgtaaaaaagactgggatccgaaaaaatacggcggctttgactccccgaccgtcgcgtacagcgtactggtcgtggctaaagtggagaaaggcaaatcgaaaaagctgaaatccgttaaagagttgctgggtattacgattatggaacgttcgagcttcgagaaaaatccgatcgacttcctggaggccaaaggctacaaggaagttaaaaaagacctgatcattaaactgccgaagtacagcctgttcgagctcgagaacggtcgcaaacgcatgctggcgagcgcgggtgagctccaaaaaggcaacgaattggctctgccgagcaagtacgtgaacttcctgtatctggcctcccattatgaaaaactgaagggtagcccggaagataacgaacaaaagcagctgttcgtggaacaacataaacactatctggacgagattattgagcaaatcagcgagttcagcaaacgcgtgattctggcggatgctaatctggacaaggttctttcagcctataataaacaccgtgataagccgatccgtgaacaggcagaaaatattattcacctgttcaccctgacgaacctgggtgcgccagcagcgtttaaatacttcgacacaacgatcgaccggaagcgttataccagcaccaaagaggttcttgacgccaccttgattcaccaaagcatcacgggtctgtatgaaacccgtattgacttgagccagttgggcggagattcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 35 - DNA, 2721 bp, synthetic construct
  source 1..2721: mol_type = other DNA
atgggcagccaccaccaccaccaccacagcggcgagaacctgtacttccagtccatggctccaaagaagaagagaaaggttggaatccacggagttccagcttctggaatgccaaaacccgctgtagagtcagaattttcaaaagttttgaaaaagcactttccgggagaacgtttccgctcgagctatatgaaaagaggcggtaagatcctggctgcccaaggcgaagaggcagttgttgcgtatctacagggcaagtccgaggaggagcctccgaacttccagccaccggcgaagtgccacgtggtgaccaaatcccgtgacttcgcggaatggccgatcatgaaggcgtcggaagcgattcaacgttatatctatgcactgtcgaccaccgaacgcgcggcctgtaaaccgggcaaaagtagtgagagccatgcagcgtggttcgccgctaccggtgtcagcaaccacggctacagccacgttcagggtctgaacctgatttttgatcataccttgggccgttacgatggtgttctgaaaaaggtccaactgcgcaacgaaaaggcgagagcgcgtttagagagcattaacgcgtctagagcagatgaaggtttaccggagattaaagccgaggaggaagaagtggctacgaacgaaaccggccacctgttgcaaccgccgggtatcaatccgagcttttacgtgtatcagaccattagcccgcaggcataccgtccgcgtgacgagatcgtgctgccgccggagtacgcaggttacgtacgtgacccgaatgctccgattccgctgggtgtggtgcgtaaccgttgcgatatccaaaaagggtgcccgggctacatcccggaatggcagcgtgaagcaggtaccgcgatcagcccgaaaacaggcaaggccgtaactgtgccgggcctgtctccgaagaaaaacaaacgtatgcgtaggtactggcgctctgagaaggagaaagctcaagatgcgctgttggttaccgtacgcattggtaccgattgggttgtcatcgacgtgcgcggtctgttgcgcaacgcccgttggcgcaccatcgctcccaaggacatcagcctgaatgcgctcctcgatttgtttaccggtgatccggtgatcgatgtgcgtcgcaatattgttacctttacgtacaccctcgacgcctgtggtacctacgcccgtaaatggaccttgaaaggcaaacagacgaaagcgaccctggacaaactgacggccactcagacggttgcgttggttgcgattgcgcttggtcagaccaacccgatttccgctggcattagccgtgttacacaagaaaatggtgcactgcaatgcgaaccgctggaccggttcaccctgccggatgacctgttgaaggacatcagcgcatatcgcattgcttgggacagaaacgaagaggaactacgcgctcgcagcgtcgaggccctgccggaggcgcaacaagcggaggtccgtgcgctggacggggtgtccaaagaaaccgcgcgtacccaactgtgcgcagatttcggcctggacccgaaacgtctgccgtgggataaaatgagctccaatactacctttatcagcgaagccttgctgtctaacagcgtgagccgtgatcaggtttttttcactccggctccgaagaagggtgctaagaaaaaagcgcctgttgaagttatgcgtaaagaccgcacttgggcacgtgcgtacaagccgcgtctgagcgtcgaagctcagaaactgaagaacgaagccctgtgggcacttaagcgcacctccccggagtatttgaagctgagccgtcgcaaagaggagctgtgtcgtcgtagcatcaactatgttatcgaaaagacccgtcgccgtacgcaatgtcagattgtgatcccggtgatcgaggatctgaatgttcgcttctttcatggttctggtaagagactgcccggttgggataatttcttcaccgctaaaaaagagaaccgttggttcatccagggcctgcacaaagcgtttagcgatctgcgtacgcaccgttccttctatgtttttgaagttcgtccggagcgcacctctattacctgtccaaagtgcggtcattgcgaggtgggtaatcgtgacggtgaggcgtttcagtgcctgagctgcggcaagacgtgcaacgcagatcttgacgtcgcgacccataatctgacccaagtggcgttgaccggtaagaccatgccgaagcgcgaggagccgcgtgacgctcagggtacagcgccagcgcgcaagactaagaaggcgagcaaatcaaaggcgccaccggcggagcgcgaagaccagaccccggcgcaagagccaagccagaccagttctggaggtagcgggggtagcggtggtagcactaacttaagtgatattatcgagaaagaaaccgggaaacagctggtgatccaagaaagcattctgatgctgcccgaagaagttgaagaagtgattggtaacaagcctgagtcggacatcttagtgcacaccgcgtacgatgaaagcacggatgagaatgtgatgctgctcacctctgatgcccccgaatacaagccgtgggctctggtgatccaggactctaacggagaaaacaaaattaagatgctctctggcggctccatggcgcctaaaaaaaaacgcaaagtggggattcacggggttccggcgtaa

SEQ ID NO: 36 - DNA, 4137 bp, synthetic construct
  source 1..4137: mol_type = other DNA
atgggcagccaccaccaccaccaccacagcggcgagaacctgtacttccagtccatggctccaaagaagaagagaaaggttggaatccacggagttccagcttctggaatgtcaaaactagaaaagtttacaaattgttactccctgtcgaaaaccctgagattcaaagcgatcccggtgggtaaaactcaagagaacattgataacaaacgtctgctggttgaggacgaaaagcgcgcggaggattacaaaggcgttaaaaagctgttggaccgttattacctgtcctttattaatgatgttctgcacagcattaaattaaagaacctaaacaattacatctccctcttccgtaaaaagacccgcaccgaaaaggaaaataaagaactggagaaccttgagattaatctgcgtaaagaaatcgctaaagcgtttaaaggcaacgagggttataaatccttatttaaaaaagacatcatcgaaacgattctcccagaattcctggacgacaaagacgagatcgcgctggttaattccttcaacggctttaccaccgcattcaccggcttttttgataaccgcgaaaacatgttctctgaagaggccaaaagcacctcaattgcgtttcgctgcattaacgaaaatctgacgcgttatatctctaacatggatatcttcgagaaggttgacgctatctttgataaacatgaagtccaggagatcaaggagaagatcctgaacagcgactacgacgtggaggatttcttcgagggtgagttcttcaattttgttttgacccaagaaggcatcgatgtctacaatgcgataattggtggttttgttaccgagagcggggagaaaattaagggtctgaacgaatatatcaacttgtacaaccagaaaaccaaacaaaaactgccgaagtttaagccgctgtacaagcaagtcttgtcggatcgtgaatcgctgagcttttacggcgaaggctacaccagcgatgaggaggtgttggaggtgttccgcaatacgttgaataagaactctgaaattttttccagcattaagaagctggaaaaactattcaagaacttcgacgagtatagcagcgcaggtatctttgtgaaaaacggtccggctattagcaccatctcgaaagacattttcggcgaatggaatgttattcgtgataagtggaatgccgaatacgacgacattcatctgaagaaaaaggcggttgtaactgaaaagtacgaggacgatcgtcgtaagtcgttcaaaaagatcggctccttctctctggagcaactgcaagagtacgccgatgccgatttgtcagtcgtggagaagctgaaagagatcatcattcaaaaagttgacgaaatctataaggtctatggtagcagtgaaaaactgtttgacgcggacttcgtgctggagaaaagcctgaagaaaaatgacgccgtggtggcaatcatgaaagatcttctggatagcgtcaagtctttcgaaaactacatcaaagcgtttttcggtgaaggcaaggaaacgaatcgtgacgagagcttctatggtgatttcgtcctagcctatgatatcctgcttaaggtggatcacatctatgatgccatccgcaactatgttacccagaaaccgtactcaaaggacaagttcaagttatacttccagaatccgcagtttatgggtggctgggataaagataaggaaaccgactatcgcgccaccattttacgttacggtagtaagtactatctggcaatcatggataaaaaatatgcgaaatgcctgcaaaagatcgacaaggacgatgttaacggcaattatgagaagatcaactacaaactgctgccgggcccaaataagatgctgccgaaggtgtttttcagcaaaaaatggatggcgtattacaatccgagcgaggacatccaaaagatctacaaaaacggtacgtttaagaagggtgatatgttcaacctcaacgattgtcataaacttattgacttcttcaaggatagcatatctcgttatccgaagtggagcaacgcgtatgactttaactttagcgaaaccgagaaatacaaagatatcgcgggtttctatcgtgaagttgaagaacagggttacaaagtgtccttcgagtccgcgagcaagaaggaggtagacaagctggtcgaggagggtaagttgtacatgtttcagatttacaataaggacttttccgacaagtcccatggtacaccgaatttgcacaccatgtatttcaagcttctgttcgacgagaacaaccacggccagatccgtctgagcggtggtgcagagctgttcatgcgtagagctagcctgaagaaagaagagttggtcgttcatccggctaattcgccgatcgcgaacaaaaatccagataaccctaaaaagactacgaccctgagctatgatgtgtataaagacaaacgctttagcgaagaccagtatgagttacacattccgatcgctatcaacaagtgcccgaagaatattttcaagatcaacaccgaagtgcgtgttcttttgaagcacgatgacaacccgtatgtgattggtattgctcgtggtgaacgtaatctgctgtacattgtggttgtggacggcaaaggaaacatcgtggaacagtacagcctgaacgaaatcattaacaatttcaacggcattcgcattaagacggattaccattctttgctggacaaaaaggagaaagagcgtttcgaggcccgtcagaactggacctccatcgagaacattaaggagctgaaggcgggttacatcagccaggtggtgcacaaaatctgcgaactggtggagaagtacgacgcggtgattgctttggcggatttgaactccggttttaagaacagccgcgttaaagtggaaaagcaggtttatcagaaattcgaaaaaatgctgattgataaattgaattacatggttgacaaaaagtctaacccgtgtgcaaccggcggtgcgctgaagggctaccaaatcaccaataagtttgaaagctttaagagcatgagcacgcaaaatggcttcattttttacattccggcgtggctgaccagcaagatcgatccgtcaaccggctttgttaatttactgaagacgaagtatacctctattgccgactctaaaaaatttattagttccttcgatagaatcatgtatgttccggaagaggacctgttcgaatttgcactggactacaagaactttagccgtaccgatgctgattatataaagaaatggaaactctacagctatggcaaccgcatccgtatctttcgcaacccgaaaaaaaacaacgtgttcgactgggaagaggtatgtctgacttcagcgtacaaggagcttttcaataaatatggtatcaactaccagcagggtgatatccgtgcactgctttgcgagcaaagcgataaagcgttctattcaagcttcatggcgctgatgagcctgatgctgcaaatgcgcaacagcatcaccggtcgtaccgatgttgcatttctgatttctccagtaaaaaacagcgatggtatcttttacgacagccgtaattacgaggcccaagaaaatgcgattttgccgaagaacgctgacgcgaatggtgcatacaacattgcaaggaaagttctgtgggcaatcggccaatttaaaaaagcggaagatgagaagctggacaaagtgaaaattgcgattagcaacaaggagtggctggagtatgcgcagacctccgttaaatcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 37 - DNA, 4947 bp, synthetic construct
  source 1..4947: mol_type = other DNA
atgggaagtcatcaccaccaccaccattcaggtgagaacctgtacttccagagcatggctccgaaaaagaaacgtaaggtgggtatccacggcgttccggcgagcggattagtaatgcaaaattataggctaatagacgctacgttgtatgtgacctttgaaccgtgcgttatgtgcgctggtgcaatgattcattctcgtatcggccgtgtcgtgttcggcgtgagaaacagcaagcgcggtgcggcgggtagcctgatgaatgttctgaactacccgggtatgaaccaccgtgttgaaattaccgagggtatcttggcagacgagtgcgccgccctgctgtgtgatttctaccgcatgccgcgtcaggtttttaacgcgcagaaaaaggcgcaaagcagcatcaatagcggcggtagctctggcggctcctccggttcggagtctggaagtgagacacccgggacttcagaatccgcgaccccggagtctggaatggataaaaagtattcaataggactagctattggcaccaacagcgttggttgggcagttatcaccgatgaatataaggtcccgagcaagaagtttaaggtcctgggcaacaccgatcgccacagcataaaaaagaaccttattggtgcgctgctgttcgattctggtgagactgccgaagcgacccgtctgaagagaaccgcgcgtcgtagatacacccggcgcaagaaccgtatctgctatctgcaagaaattttcagcaacgagatggctaaggtcgacgactccttctttcatcgcctggaggaaagctttctggttgaggaagacaaaaagcacgagcgtcatccgattttcggtaacattgttgatgaagttgcctaccacgaaaaataccccacaatttatcatctgcgtaaaaaactggtagactccaccgataaggcggatttacgccttatctatttggcgctggcgcatatgattaagttccgcggtcattttttgatcgagggcgacctgaatccggataacagcgacgttgacaaactgtttatccagctggtgcagacctataaccagttgttcgaagagaacccgattaatgcgtctggcgtggacgcgaaggcgatcctgagcgctcgtctgtccaagtcacgccgcctggagaacttgatcgcgcaactgccgggtgagaagaaaaacggcttgttcggcaatctgattgctctgtccctaggtttgaccccgaatttcaaatctaattttgatctggcggaagatgcgaaattacagttgagcaaggacacttacgatgacgatctggacaacctgctcgctcagatcggcgatcagtatgccgatcttttcctggccgcgaagaacctgagcgatgccatcttgctatcggacatcttacgtgttaacaccgaaatcaccaaagctccgctaagcgcgtccatgattaaacgttacgatgaacaccaccaggatctgacgctgctgaaagcgttggtgcgccagcaactgccggaaaaatacaaggaaattttcttcgaccaaagcaagaacggttatgcaggctatatcgacggtggtgcgtcgcaagaagaattttataaattcatcaaaccgattttggagaagatggatggtaccgaggagctgctggtaaaattgaatcgggaagacctcttgcgaaagcagcgtactttcgataatggtagcatcccgcaccagattcatctgggcgaacttcacgcaatcctacgtcgtcaagaggatttctacccgtttctgaaggataaccgtgaaaagatcgagaagattctgacctttcgtattccgtattatgtgggtccgctggcgagaggcaacagccgttttgcgtggatgacccgcaagtcggaagagactatcacgccgtggaatttcgaggaggtcgtggacaagggtgcatcagcgcaatcttttattgaacgtatgaccaattttgataaaaaccttccaaatgaaaaagtgttgccgaagcacagcttgctgtacgagtatttcaccgtttacaatgaactgacgaaagtcaagtacgttaccgaaggtatgcgtaagccagcctttctgtctggggagcaaaaaaaagccattgtggatctgctgttcaagaccaatcgcaaggttaccgtcaagcaactgaaagaggattacttcaaaaaaatcgaatgttttgactccgtggaaatcagcggcgttgaagaccgctttaatgcatctctgggcacctaccacgatctactcaagatcatcaaggataaggacttcctggacaacgaagaaaatgaggatatcttggaagatattgtgctgactttaacgttgttcgaggatcgtgagatgattgaagaacgcctcaaaacgtacgcacatctgtttgacgataaggtgatgaaacagctaaagcgccgtcgttacaccggttggggtcgtctgtctagaaagctgattaacggcattcgtgacaaacaatctggcaagaccatcctggacttcctcaaatccgacggcttcgcaaatcgtaatttcatgcaactgatccatgatgatagcctgacctttaaagaagatattcagaaggctcaagtgtccggccagggtgactcgctgcatgagcatatcgcgaatctggctggctcccctgcgataaaaaaaggtattctccaaacggtgaaagttgtggatgaactggttaaagtcatgggtcgtcataaaccggaaaacatcgtgattgagatggcacgtgagaaccagaccacccagaaaggtcaaaaaaacagtcgtgagcgtatgaaacgtatcgaagaggggattaaggagttaggcagccagattctgaaggagcacccggttgagaacacccagctgcaaaacgagaagctctacctgtattacctgcaaaacggccgtgacatgtatgttgaccaggagctggacatcaatcgtctcagcgactacgacgtggatgctatcgtgccgcagagctttctaaaagatgattccatcgacaataaagtgctcacccgtagcgacaagaaccgtggtaaaagcgacaacgtgccgagcgaggaagtagttaagaaaatgaaaaactactggcgtcagctattgaacgccaagcttatcacccagcgtaagtttgacaacttgacgaaggcagagcgtggcggcctgagcgagctagacaaggcgggttttattaaacgccagctggtggaaactcgccagattaccaaacacgttgcgcaaatcttggactcacgcatgaatacgaagtacgacgaaaacgataaattgatccgcgaggtgaaggtcataaccctgaaatctaaactggttagcgatttccggaaggactttcaattttataaagtccgggagattaacaactatcaccatgcacacgacgcttacttaaatgcggttgttggcacggccctgatcaagaagtacccgaaactggaaagcgagtttgtttatggtgactacaaggtgtatgacgttcgcaagatgattgcaaaatccgaacaagagattggtaaggcgacggctaagtattttttctacagcaatatcatgaacttcttcaagactgaaatcaccctggcgaacggtgaaattcgtaaacgtccgttgatcgaaaccaacggtgaaaccggtgaaatcgtttgggataaaggtagggattttgcgaccgttcgtaaggtcctgagcatgccgcaggttaacatcgtgaaaaagaccgaagtgcagaccggcggtttctccaaggagagtatcttaccgaagcgcaacagtgacaagctgatcgcgcgtaaaaaagactgggatccgaaaaaatacggcggctttgactccccgaccgtcgcgtacagcgtactggtcgtggctaaagtggagaaaggcaaatcgaaaaagctgaaatccgttaaagagttgctgggtattacgattatggaacgttcgagcttcgagaaaaatccgatcgacttcctggaggccaaaggctacaaggaagttaaaaaagacctgatcattaaactgccgaagtacagcctgttcgagctcgagaacggtcgcaaacgcatgctggcgagcgcgggtgagctccaaaaaggcaacgaattggctctgccgagcaagtacgtgaacttcctgtatctggcctcccattatgaaaaactgaagggtagcccggaagataacgaacaaaagcagctgttcgtggaacaacataaacactatctggacgagattattgagcaaatcagcgagttcagcaaacgcgtgattctggcggatgctaatctggacaaggttctttcagcctataataaacaccgtgataagccgatccgtgaacaggcagaaaatattattcacctgttcaccctgacgaacctgggtgcgccagcagcgtttaaatacttcgacacaacgatcgaccggaagcgttataccagcaccaaagaggttcttgacgccaccttgattcaccaaagcatcacgggtctgtatgaaacccgtattgacttgagccagttgggcggagattcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 38 - DNA, 3108 bp, synthetic construct
  source 1..3108: mol_type = other DNA
atgggaagtcatcaccaccaccaccattcaggtgagaacctgtacttccagagcatggctccgaaaaagaaacgtaaggtgggtatccacggcgttccggcgagcggattagtaatgcaaaattataggctaatagacgctacgttgtatgtgacctttgaaccgtgcgttatgtgcgctggtgcaatgattcattctcgtatcggccgtgtcgtgttcggcgtgagaaacagcaagcgcggtgcggcgggtagcctgatgaatgttctgaactacccgggtatgaaccaccgtgttgaaattaccgagggtatcttggcagacgagtgcgccgccctgctgtgtgatttctaccgcatgccgcgtcaggtttttaacgcgcagaaaaaggcgcaaagcagcatcaatagcggcggtagctctggcggctcctccggttcggagtctggaagtgagacacccgggacttcagaatccgcgaccccggagtctggaatgccaaaacccgctgtagagtcagaattttcaaaagttttgaaaaagcactttccgggagaacgtttccgctcgagctatatgaaaagaggcggtaagatcctggctgcccaaggcgaagaggcagttgttgcgtatctacagggcaagtccgaggaggagcctccgaacttccagccaccggcgaagtgccacgtggtgaccaaatcccgtgacttcgcggaatggccgatcatgaaggcgtcggaagcgattcaacgttatatctatgcactgtcgaccaccgaacgcgcggcctgtaaaccgggcaaaagtagtgagagccatgcagcgtggttcgccgctaccggtgtcagcaaccacggctacagccacgttcagggtctgaacctgatttttgatcataccttgggccgttacgatggtgttctgaaaaaggtccaactgcgcaacgaaaaggcgagagcgcgtttagagagcattaacgcgtctagagcagatgaaggtttaccggagattaaagccgaggaggaagaagtggctacgaacgaaaccggccacctgttgcaaccgccgggtatcaatccgagcttttacgtgtatcagaccattagcccgcaggcataccgtccgcgtgacgagatcgtgctgccgccggagtacgcaggttacgtacgtgacccgaatgctccgattccgctgggtgtggtgcgtaaccgttgcgatatccaaaaagggtgcccgggctacatcccggaatggcagcgtgaagcaggtaccgcgatcagcccgaaaacaggcaaggccgtaactgtgccgggcctgtctccgaagaaaaacaaacgtatgcgtaggtactggcgctctgagaaggagaaagctcaagatgcgctgttggttaccgtacgcattggtaccgattgggttgtcatcgacgtgcgcggtctgttgcgcaacgcccgttggcgcaccatcgctcccaaggacatcagcctgaatgcgctcctcgatttgtttaccggtgatccggtgatcgatgtgcgtcgcaatattgttacctttacgtacaccctcgacgcctgtggtacctacgcccgtaaatggaccttgaaaggcaaacagacgaaagcgaccctggacaaactgacggccactcagacggttgcgttggttgcgattgcgcttggtcagaccaacccgatttccgctggcattagccgtgttacacaagaaaatggtgcactgcaatgcgaaccgctggaccggttcaccctgccggatgacctgttgaaggacatcagcgcatatcgcattgcttgggacagaaacgaagaggaactacgcgctcgcagcgtcgaggccctgccggaggcgcaacaagcggaggtccgtgcgctggacggggtgtccaaagaaaccgcgcgtacccaactgtgcgcagatttcggcctggacccgaaacgtctgccgtgggataaaatgagctccaatactacctttatcagcgaagccttgctgtctaacagcgtgagccgtgatcaggtttttttcactccggctccgaagaagggtgctaagaaaaaagcgcctgttgaagttatgcgtaaagaccgcacttgggcacgtgcgtacaagccgcgtctgagcgtcgaagctcagaaactgaagaacgaagccctgtgggcacttaagcgcacctccccggagtatttgaagctgagccgtcgcaaagaggagctgtgtcgtcgtagcatcaactatgttatcgaaaagacccgtcgccgtacgcaatgtcagattgtgatcccggtgatcgaggatctgaatgttcgcttctttcatggttctggtaagagactgcccggttgggataatttcttcaccgctaaaaaagagaaccgttggttcatccagggcctgcacaaagcgtttagcgatctgcgtacgcaccgttccttctatgtttttgaagttcgtccggagcgcacctctattacctgtccaaagtgcggtcattgcgaggtgggtaatcgtgacggtgaggcgtttcagtgcctgagctgcggcaagacgtgcaacgcagatcttgacgtcgcgacccataatctgacccaagtggcgttgaccggtaagaccatgccgaagcgcgaggagccgcgtgacgctcagggtacagcgccagcgcgcaagactaagaaggcgagcaaatcaaaggcgccaccggcggagcgcgaagaccagaccccggcgcaagagccaagccagaccagttctggaggtagcgggggtagcggtggtagcactaacttaagtgatattatcgagaaagaaaccgggaaacagctggtgatccaagaaagcattctgatgctgcccgaagaagttgaagaagtgattggtaacaagcctgagtcggacatcttagtgcacaccgcgtacgatgaaagcacggatgagaatgtgatgctgctcacctctgatgcccccgaatacaagccgtgggctctggtgatccaggactctaacggagaaaacaaaattaagatgctctctggcggctccatggcgcctaaaaaaaaacgcaaagtggggattcacggggttccggcgtaa

SEQ ID NO: 39 - DNA, 4524 bp, synthetic construct
  source 1..4524: mol_type = other DNA
atgggaagtcatcaccaccaccaccattcaggtgagaacctgtacttccagagcatggctccgaaaaagaaacgtaaggtgggtatccacggcgttccggcgagcggattagtaatgcaaaattataggctaatagacgctacgttgtatgtgacctttgaaccgtgcgttatgtgcgctggtgcaatgattcattctcgtatcggccgtgtcgtgttcggcgtgagaaacagcaagcgcggtgcggcgggtagcctgatgaatgttctgaactacccgggtatgaaccaccgtgttgaaattaccgagggtatcttggcagacgagtgcgccgccctgctgtgtgatttctaccgcatgccgcgtcaggtttttaacgcgcagaaaaaggcgcaaagcagcatcaatagcggcggtagctctggcggctcctccggttcggagtctggaagtgagacacccgggacttcagaatccgcgaccccggagtctggaatgtcaaaactagaaaagtttacaaattgttactccctgtcgaaaaccctgagattcaaagcgatcccggtgggtaaaactcaagagaacattgataacaaacgtctgctggttgaggacgaaaagcgcgcggaggattacaaaggcgttaaaaagctgttggaccgttattacctgtcctttattaatgatgttctgcacagcattaaattaaagaacctaaacaattacatctccctcttccgtaaaaagacccgcaccgaaaaggaaaataaagaactggagaaccttgagattaatctgcgtaaagaaatcgctaaagcgtttaaaggcaacgagggttataaatccttatttaaaaaagacatcatcgaaacgattctcccagaattcctggacgacaaagacgagatcgcgctggttaattccttcaacggctttaccaccgcattcaccggcttttttgataaccgcgaaaacatgttctctgaagaggccaaaagcacctcaattgcgtttcgctgcattaacgaaaatctgacgcgttatatctctaacatggatatcttcgagaaggttgacgctatctttgataaacatgaagtccaggagatcaaggagaagatcctgaacagcgactacgacgtggaggatttcttcgagggtgagttcttcaattttgttttgacccaagaaggcatcgatgtctacaatgcgataattggtggttttgttaccgagagcggggagaaaattaagggtctgaacgaatatatcaacttgtacaaccagaaaaccaaacaaaaactgccgaagtttaagccgctgtacaagcaagtcttgtcggatcgtgaatcgctgagcttttacggcgaaggctacaccagcgatgaggaggtgttggaggtgttccgcaatacgttgaataagaactctgaaattttttccagcattaagaagctggaaaaactattcaagaacttcgacgagtatagcagcgcaggtatctttgtgaaaaacggtccggctattagcaccatctcgaaagacattttcggcgaatggaatgttattcgtgataagtggaatgccgaatacgacgacattcatctgaagaaaaaggcggttgtaactgaaaagtacgaggacgatcgtcgtaagtcgttcaaaaagatcggctccttctctctggagcaactgcaagagtacgccgatgccgatttgtcagtcgtggagaagctgaaagagatcatcattcaaaaagttgacgaaatctataaggtctatggtagcagtgaaaaactgtttgacgcggacttcgtgctggagaaaagcctgaagaaaaatgacgccgtggtggcaatcatgaaagatcttctggatagcgtcaagtctttcgaaaactacatcaaagcgtttttcggtgaaggcaaggaaacgaatcgtgacgagagcttctatggtgatttcgtcctagcctatgatatcctgcttaaggtggatcacatctatgatgccatccgcaactatgttacccagaaaccgtactcaaaggacaagttcaagttatacttccagaatccgcagtttatgggtggctgggataaagataaggaaaccgactatcgcgccaccattttacgttacggtagtaagtactatctggcaatcatggataaaaaatatgcgaaatgcctgcaaaagatcgacaaggacgatgttaacggcaattatgagaagatcaactacaaactgctgccgggcccaaataagatgctgccgaaggtgtttttcagcaaaaaatggatggcgtattacaatccgagcgaggacatccaaaagatctacaaaaacggtacgtttaagaagggtgatatgttcaacctcaacgattgtcataaacttattgacttcttcaaggatagcatatctcgttatccgaagtggagcaacgcgtatgactttaactttagcgaaaccgagaaatacaaagatatcgcgggtttctatcgtgaagttgaagaacagggttacaaagtgtccttcgagtccgcgagcaagaaggaggtagacaagctggtcgaggagggtaagttgtacatgtttcagatttacaataaggacttttccgacaagtcccatggtacaccgaatttgcacaccatgtatttcaagcttctgttcgacgagaacaaccacggccagatccgtctgagcggtggtgcagagctgttcatgcgtagagctagcctgaagaaagaagagttggtcgttcatccggctaattcgccgatcgcgaacaaaaatccagataaccctaaaaagactacgaccctgagctatgatgtgtataaagacaaacgctttagcgaagaccagtatgagttacacattccgatcgctatcaacaagtgcccgaagaatattttcaagatcaacaccgaagtgcgtgttcttttgaagcacgatgacaacccgtatgtgattggtattgctcgtggtgaacgtaatctgctgtacattgtggttgtggacggcaaaggaaacatcgtggaacagtacagcctgaacgaaatcattaacaatttcaacggcattcgcattaagacggattaccattctttgctggacaaaaaggagaaagagcgtttcgaggcccgtcagaactggacctccatcgagaacattaaggagctgaaggcgggttacatcagccaggtggtgcacaaaatctgcgaactggtggagaagtacgacgcggtgattgctttggcggatttgaactccggttttaagaacagccgcgttaaagtggaaaagcaggtttatcagaaattcgaaaaaatgctgattgataaattgaattacatggttgacaaaaagtctaacccgtgtgcaaccggcggtgcgctgaagggctaccaaatcaccaataagtttgaaagctttaagagcatgagcacgcaaaatggcttcattttttacattccggcgtggctgaccagcaagatcgatccgtcaaccggctttgttaatttactgaagacgaagtatacctctattgccgactctaaaaaatttattagttccttcgatagaatcatgtatgttccggaagaggacctgttcgaatttgcactggactacaagaactttagccgtaccgatgctgattatataaagaaatggaaactctacagctatggcaaccgcatccgtatctttcgcaacccgaaaaaaaacaacgtgttcgactgggaagaggtatgtctgacttcagcgtacaaggagcttttcaataaatatggtatcaactaccagcagggtgatatccgtgcactgctttgcgagcaaagcgataaagcgttctattcaagcttcatggcgctgatgagcctgatgctgcaaatgcgcaacagcatcaccggtcgtaccgatgttgcatttctgatttctccagtaaaaaacagcgatggtatcttttacgacagccgtaattacgaggcccaagaaaatgcgattttgccgaagaacgctgacgcgaatggtgcatacaacattgcaaggaaagttctgtgggcaatcggccaatttaaaaaagcggaagatgagaagctggacaaagtgaaaattgcgattagcaacaaggagtggctggagtatgcgcagacctccgttaaatcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 40 - DNA, 5229 bp, synthetic construct
  source 1..5229: mol_type = other DNA
atgggaagtcatcaccaccaccaccattcaggtgagaacctgtacttccagagcatggctccgaaaaagaaacgtaaggtgggtatccacggcgttccggcgatgacagatgctgaatatgtaaggatacacgagaagttggacatctacacctttaaaaagcagttctttaacaacaaaaagagcgtcagccatcgttgctatgttctgtttgagttgaagcgccgtggcgaacgtcgtgcgtgcttttggggttatgctgtgaacaaaccgcagtctggtactgaacgcggtattcatgcagaaattttcagcattcgtaaggtggaagaatacctgcgcgataacccaggtcagttcaccattaactggtacagcagctggtccccgtgcgcggattgcgctgagaagatcttggagtggtacaatcaagagctgcgtggtaatggccataccctgaaaatctgggcgtgcaaactgtattacgaaaagaacgcgcgtaatcaaattggtctgtggaatctgcgtgacaacggcgtgggcctgaatgttatggtttccgagcactatcagtgttgtcgtaaaatcttcatccaatcctctcacaaccaattgaacgagaaccgctggctggagaagaccctgaagcgcgccgagaaaagacgtagcgaactctcgatcatgattcaggttaaaatcttacacacgaccaaaagcccggcagtgtctggaagtgagacacccgggacttcagaatccgcgaccccggagtctggaatggataaaaagtattcaataggactagctattggcaccaacagcgttggttgggcagttatcaccgatgaatataaggtcccgagcaagaagtttaaggtcctgggcaacaccgatcgccacagcataaaaaagaaccttattggtgcgctgctgttcgattctggtgagactgccgaagcgacccgtctgaagagaaccgcgcgtcgtagatacacccggcgcaagaaccgtatctgctatctgcaagaaattttcagcaacgagatggctaaggtcgacgactccttctttcatcgcctggaggaaagctttctggttgaggaagacaaaaagcacgagcgtcatccgattttcggtaacattgttgatgaagttgcctaccacgaaaaataccccacaatttatcatctgcgtaaaaaactggtagactccaccgataaggcggatttacgccttatctatttggcgctggcgcatatgattaagttccgcggtcattttttgatcgagggcgacctgaatccggataacagcgacgttgacaaactgtttatccagctggtgcagacctataaccagttgttcgaagagaacccgattaatgcgtctggcgtggacgcgaaggcgatcctgagcgctcgtctgtccaagtcacgccgcctggagaacttgatcgcgcaactgccgggtgagaagaaaaacggcttgttcggcaatctgattgctctgtccctaggtttgaccccgaatttcaaatctaattttgatctggcggaagatgcgaaattacagttgagcaaggacacttacgatgacgatctggacaacctgctcgctcagatcggcgatcagtatgccgatcttttcctggccgcgaagaacctgagcgatgccatcttgctatcggacatcttacgtgttaacaccgaaatcaccaaagctccgctaagcgcgtccatgattaaacgttacgatgaacaccaccaggatctgacgctgctgaaagcgttggtgcgccagcaactgccggaaaaatacaaggaaattttcttcgaccaaagcaagaacggttatgcaggctatatcgacggtggtgcgtcgcaagaagaattttataaattcatcaaaccgattttggagaagatggatggtaccgaggagctgctggtaaaattgaatcgggaagacctcttgcgaaagcagcgtactttcgataatggtagcatcccgcaccagattcatctgggcgaacttcacgcaatcctacgtcgtcaagaggatttctacccgtttctgaaggataaccgtgaaaagatcgagaagattctgacctttcgtattccgtattatgtgggtccgctggcgagaggcaacagccgttttgcgtggatgacccgcaagtcggaagagactatcacgccgtggaatttcgaggaggtcgtggacaagggtgcatcagcgcaatcttttattgaacgtatgaccaattttgataaaaaccttccaaatgaaaaagtgttgccgaagcacagcttgctgtacgagtatttcaccgtttacaatgaactgacgaaagtcaagtacgttaccgaaggtatgcgtaagccagcctttctgtctggggagcaaaaaaaagccattgtggatctgctgttcaagaccaatcgcaaggttaccgtcaagcaactgaaagaggattacttcaaaaaaatcgaatgttttgactccgtggaaatcagcggcgttgaagaccgctttaatgcatctctgggcacctaccacgatctactcaagatcatcaaggataaggacttcctggacaacgaagaaaatgaggatatcttggaagatattgtgctgactttaacgttgttcgaggatcgtgagatgattgaagaacgcctcaaaacgtacgcacatctgtttgacgataaggtgatgaaacagctaaagcgccgtcgttacaccggttggggtcgtctgtctagaaagctgattaacggcattcgtgacaaacaatctggcaagaccatcctggacttcctcaaatccgacggcttcgcaaatcgtaatttcatgcaactgatccatgatgatagcctgacctttaaagaagatattcagaaggctcaagtgtccggccagggtgactcgctgcatgagcatatcgcgaatctggctggctcccctgcgataaaaaaaggtattctccaaacggtgaaagttgtggatgaactggttaaagtcatgggtcgtcataaaccggaaaacatcgtgattgagatggcacgtgagaaccagaccacccagaaaggtcaaaaaaacagtcgtgagcgtatgaaacgtatcgaagaggggattaaggagttaggcagccagattctgaaggagcacccggttgagaacacccagctgcaaaacgagaagctctacctgtattacctgcaaaacggccgtgacatgtatgttgaccaggagctggacatcaatcgtctcagcgactacgacgtggatgctatcgtgccgcagagctttctaaaagatgattccatcgacaataaagtgctcacccgtagcgacaagaaccgtggtaaaagcgacaacgtgccgagcgaggaagtagttaagaaaatgaaaaactactggcgtcagctattgaacgccaagcttatcacccagcgtaagtttgacaacttgacgaaggcagagcgtggcggcctgagcgagctagacaaggcgggttttattaaacgccagctggtggaaactcgccagattaccaaacacgttgcgcaaatcttggactcacgcatgaatacgaagtacgacgaaaacgataaattgatccgcgaggtgaaggtcataaccctgaaatctaaactggttagcgatttccggaaggactttcaattttataaagtccgggagattaacaactatcaccatgcacacgacgcttacttaaatgcggttgttggcacggccctgatcaagaagtacccgaaactggaaagcgagtttgtttatggtgactacaaggtgtatgacgttcgcaagatgattgcaaaatccgaacaagagattggtaaggcgacggctaagtattttttctacagcaatatcatgaacttcttcaagactgaaatcaccctggcgaacggtgaaattcgtaaacgtccgttgatcgaaaccaacggtgaaaccggtgaaatcgtttgggataaaggtagggattttgcgaccgttcgtaaggtcctgagcatgccgcaggttaacatcgtgaaaaagaccgaagtgcagaccggcggtttctccaaggagagtatcttaccgaagcgcaacagtgacaagctgatcgcgcgtaaaaaagactgggatccgaaaaaatacggcggctttgactccccgaccgtcgcgtacagcgtactggtcgtggctaaagtggagaaaggcaaatcgaaaaagctgaaatccgttaaagagttgctgggtattacgattatggaacgttcgagcttcgagaaaaatccgatcgacttcctggaggccaaaggctacaaggaagttaaaaaagacctgatcattaaactgccgaagtacagcctgttcgagctcgagaacggtcgcaaacgcatgctggcgagcgcgggtgagctccaaaaaggcaacgaattggctctgccgagcaagtacgtgaacttcctgtatctggcctcccattatgaaaaactgaagggtagcccggaagataacgaacaaaagcagctgttcgtggaacaacataaacactatctggacgagattattgagcaaatcagcgagttcagcaaacgcgtgattctggcggatgctaatctggacaaggttctttcagcctataataaacaccgtgataagccgatccgtgaacaggcagaaaatattattcacctgttcaccctgacgaacctgggtgcgccagcagcgtttaaatacttcgacacaacgatcgaccggaagcgttataccagcaccaaagaggttcttgacgccaccttgattcaccaaagcatcacgggtctgtatgaaacccgtattgacttgagccagttgggcggagattcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 41 - DNA, 3390 bp, synthetic construct
  source 1..3390: mol_type = other DNA
atgggaagtcatcaccaccaccaccattcaggtgagaacctgtacttccagagcatggctccgaaaaagaaacgtaaggtgggtatccacggcgttccggcgatgacagatgctgaatatgtaaggatacacgagaagttggacatctacacctttaaaaagcagttctttaacaacaaaaagagcgtcagccatcgttgctatgttctgtttgagttgaagcgccgtggcgaacgtcgtgcgtgcttttggggttatgctgtgaacaaaccgcagtctggtactgaacgcggtattcatgcagaaattttcagcattcgtaaggtggaagaatacctgcgcgataacccaggtcagttcaccattaactggtacagcagctggtccccgtgcgcggattgcgctgagaagatcttggagtggtacaatcaagagctgcgtggtaatggccataccctgaaaatctgggcgtgcaaactgtattacgaaaagaacgcgcgtaatcaaattggtctgtggaatctgcgtgacaacggcgtgggcctgaatgttatggtttccgagcactatcagtgttgtcgtaaaatcttcatccaatcctctcacaaccaattgaacgagaaccgctggctggagaagaccctgaagcgcgccgagaaaagacgtagcgaactctcgatcatgattcaggttaaaatcttacacacgaccaaaagcccggcagtgtctggaagtgagacacccgggacttcagaatccgcgaccccggagtctggaatgccaaaacccgctgtagagtcagaattttcaaaagttttgaaaaagcactttccgggagaacgtttccgctcgagctatatgaaaagaggcggtaagatcctggctgcccaaggcgaagaggcagttgttgcgtatctacagggcaagtccgaggaggagcctccgaacttccagccaccggcgaagtgccacgtggtgaccaaatcccgtgacttcgcggaatggccgatcatgaaggcgtcggaagcgattcaacgttatatctatgcactgtcgaccaccgaacgcgcggcctgtaaaccgggcaaaagtagtgagagccatgcagcgtggttcgccgctaccggtgtcagcaaccacggctacagccacgttcagggtctgaacctgatttttgatcataccttgggccgttacgatggtgttctgaaaaaggtccaactgcgcaacgaaaaggcgagagcgcgtttagagagcattaacgcgtctagagcagatgaaggtttaccggagattaaagccgaggaggaagaagtggctacgaacgaaaccggccacctgttgcaaccgccgggtatcaatccgagcttttacgtgtatcagaccattagcccgcaggcataccgtccgcgtgacgagatcgtgctgccgccggagtacgcaggttacgtacgtgacccgaatgctccgattccgctgggtgtggtgcgtaaccgttgcgatatccaaaaagggtgcccgggctacatcccggaatggcagcgtgaagcaggtaccgcgatcagcccgaaaacaggcaaggccgtaactgtgccgggcctgtctccgaagaaaaacaaacgtatgcgtaggtactggcgctctgagaaggagaaagctcaagatgcgctgttggttaccgtacgcattggtaccgattgggttgtcatcgacgtgcgcggtctgttgcgcaacgcccgttggcgcaccatcgctcccaaggacatcagcctgaatgcgctcctcgatttgtttaccggtgatccggtgatcgatgtgcgtcgcaatattgttacctttacgtacaccctcgacgcctgtggtacctacgcccgtaaatggaccttgaaaggcaaacagacgaaagcgaccctggacaaactgacggccactcagacggttgcgttggttgcgattgcgcttggtcagaccaacccgatttccgctggcattagccgtgttacacaagaaaatggtgcactgcaatgcgaaccgctggaccggttcaccctgccggatgacctgttgaaggacatcagcgcatatcgcattgcttgggacagaaacgaagaggaactacgcgctcgcagcgtcgaggccctgccggaggcgcaacaagcggaggtccgtgcgctggacggggtgtccaaagaaaccgcgcgtacccaactgtgcgcagatttcggcctggacccgaaacgtctgccgtgggataaaatgagctccaatactacctttatcagcgaagccttgctgtctaacagcgtgagccgtgatcaggtttttttcactccggctccgaagaagggtgctaagaaaaaagcgcctgttgaagttatgcgtaaagaccgcacttgggcacgtgcgtacaagccgcgtctgagcgtcgaagctcagaaactgaagaacgaagccctgtgggcacttaagcgcacctccccggagtatttgaagctgagccgtcgcaaagaggagctgtgtcgtcgtagcatcaactatgttatcgaaaagacccgtcgccgtacgcaatgtcagattgtgatcccggtgatcgaggatctgaatgttcgcttctttcatggttctggtaagagactgcccggttgggataatttcttcaccgctaaaaaagagaaccgttggttcatccagggcctgcacaaagcgtttagcgatctgcgtacgcaccgttccttctatgtttttgaagttcgtccggagcgcacctctattacctgtccaaagtgcggtcattgcgaggtgggtaatcgtgacggtgaggcgtttcagtgcctgagctgcggcaagacgtgcaacgcagatcttgacgtcgcgacccataatctgacccaagtggcgttgaccggtaagaccatgccgaagcgcgaggagccgcgtgacgctcagggtacagcgccagcgcgcaagactaagaaggcgagcaaatcaaaggcgccaccggcggagcgcgaagaccagaccccggcgcaagagccaagccagaccagttctggaggtagcgggggtagcggtggtagcactaacttaagtgatattatcgagaaagaaaccgggaaacagctggtgatccaagaaagcattctgatgctgcccgaagaagttgaagaagtgattggtaacaagcctgagtcggacatcttagtgcacaccgcgtacgatgaaagcacggatgagaatgtgatgctgctcacctctgatgcccccgaatacaagccgtgggctctggtgatccaggactctaacggagaaaacaaaattaagatgctctctggcggctccatggcgcctaaaaaaaaacgcaaagtggggattcacggggttccggcgtaa

SEQ ID NO: 42 - DNA, 4806 bp, synthetic construct
  source 1..4806: mol_type = other DNA
atgggaagtcatcaccaccaccaccattcaggtgagaacctgtacttccagagcatggctccgaaaaagaaacgtaaggtgggtatccacggcgttccggcgatgacagatgctgaatatgtaaggatacacgagaagttggacatctacacctttaaaaagcagttctttaacaacaaaaagagcgtcagccatcgttgctatgttctgtttgagttgaagcgccgtggcgaacgtcgtgcgtgcttttggggttatgctgtgaacaaaccgcagtctggtactgaacgcggtattcatgcagaaattttcagcattcgtaaggtggaagaatacctgcgcgataacccaggtcagttcaccattaactggtacagcagctggtccccgtgcgcggattgcgctgagaagatcttggagtggtacaatcaagagctgcgtggtaatggccataccctgaaaatctgggcgtgcaaactgtattacgaaaagaacgcgcgtaatcaaattggtctgtggaatctgcgtgacaacggcgtgggcctgaatgttatggtttccgagcactatcagtgttgtcgtaaaatcttcatccaatcctctcacaaccaattgaacgagaaccgctggctggagaagaccctgaagcgcgccgagaaaagacgtagcgaactctcgatcatgattcaggttaaaatcttacacacgaccaaaagcccggcagtgtctggaagtgagacacccgggacttcagaatccgcgaccccggagtctggaatgtcaaaactagaaaagtttacaaattgttactccctgtcgaaaaccctgagattcaaagcgatcccggtgggtaaaactcaagagaacattgataacaaacgtctgctggttgaggacgaaaagcgcgcggaggattacaaaggcgttaaaaagctgttggaccgttattacctgtcctttattaatgatgttctgcacagcattaaattaaagaacctaaacaattacatctccctcttccgtaaaaagacccgcaccgaaaaggaaaataaagaactggagaaccttgagattaatctgcgtaaagaaatcgctaaagcgtttaaaggcaacgagggttataaatccttatttaaaaaagacatcatcgaaacgattctcccagaattcctggacgacaaagacgagatcgcgctggttaattccttcaacggctttaccaccgcattcaccggcttttttgataaccgcgaaaacatgttctctgaagaggccaaaagcacctcaattgcgtttcgctgcattaacgaaaatctgacgcgttatatctctaacatggatatcttcgagaaggttgacgctatctttgataaacatgaagtccaggagatcaaggagaagatcctgaacagcgactacgacgtggaggatttcttcgagggtgagttcttcaattttgttttgacccaagaaggcatcgatgtctacaatgcgataattggtggttttgttaccgagagcggggagaaaattaagggtctgaacgaatatatcaacttgtacaaccagaaaaccaaacaaaaactgccgaagtttaagccgctgtacaagcaagtcttgtcggatcgtgaatcgctgagcttttacggcgaaggctacaccagcgatgaggaggtgttggaggtgttccgcaatacgttgaataagaactctgaaattttttccagcattaagaagctggaaaaactattcaagaacttcgacgagtatagcagcgcaggtatctttgtgaaaaacggtccggctattagcaccatctcgaaagacattttcggcgaatggaatgttattcgtgataagtggaatgccgaatacgacgacattcatctgaagaaaaaggcggttgtaactgaaaagtacgaggacgatcgtcgtaagtcgttcaaaaagatcggctccttctctctggagcaactgcaagagtacgccgatgccgatttgtcagtcgtggagaagctgaaagagatcatcattcaaaaagttgacgaaatctataaggtctatggtagcagtgaaaaactgtttgacgcggacttcgtgctggagaaaagcctgaagaaaaatgacgccgtggtggcaatcatgaaagatcttctggatagcgtcaagtctttcgaaaactacatcaaagcgtttttcggtgaaggcaaggaaacgaatcgtgacgagagcttctatggtgatttcgtcctagcctatgatatcctgcttaaggtggatcacatctatgatgccatccgcaactatgttacccagaaaccgtactcaaaggacaagttcaagttatacttccagaatccgcagtttatgggtggctgggataaagataaggaaaccgactatcgcgccaccattttacgttacggtagtaagtactatctggcaatcatggataaaaaatatgcgaaatgcctgcaaaagatcgacaaggacgatgttaacggcaattatgagaagatcaactacaaactgctgccgggcccaaataagatgctgccgaaggtgtttttcagcaaaaaatggatggcgtattacaatccgagcgaggacatccaaaagatctacaaaaacggtacgtttaagaagggtgatatgttcaacctcaacgattgtcataaacttattgacttcttcaaggatagcatatctcgttatccgaagtggagcaacgcgtatgactttaactttagcgaaaccgagaaatacaaagatatcgcgggtttctatcgtgaagttgaagaacagggttacaaagtgtccttcgagtccgcgagcaagaaggaggtagacaagctggtcgaggagggtaagttgtacatgtttcagatttacaataaggacttttccgacaagtcccatggtacaccgaatttgcacaccatgtatttcaagcttctgttcgacgagaacaaccacggccagatccgtctgagcggtggtgcagagctgttcatgcgtagagctagcctgaagaaagaagagttggtcgttcatccggctaattcgccgatcgcgaacaaaaatccagataaccctaaaaagactacgaccctgagctatgatgtgtataaagacaaacgctttagcgaagaccagtatgagttacacattccgatcgctatcaacaagtgcccgaagaatattttcaagatcaacaccgaagtgcgtgttcttttgaagcacgatgacaacccgtatgtgattggtattgctcgtggtgaacgtaatctgctgtacattgtggttgtggacggcaaaggaaacatcgtggaacagtacagcctgaacgaaatcattaacaatttcaacggcattcgcattaagacggattaccattctttgctggacaaaaaggagaaagagcgtttcgaggcccgtcagaactggacctccatcgagaacattaaggagctgaaggcgggttacatcagccaggtggtgcacaaaatctgcgaactggtggagaagtacgacgcggtgattgctttggcggatttgaactccggttttaagaacagccgcgttaaagtggaaaagcaggtttatcagaaattcgaaaaaatgctgattgataaattgaattacatggttgacaaaaagtctaacccgtgtgcaaccggcggtgcgctgaagggctaccaaatcaccaataagtttgaaagctttaagagcatgagcacgcaaaatggcttcattttttacattccggcgtggctgaccagcaagatcgatccgtcaaccggctttgttaatttactgaagacgaagtatacctctattgccgactctaaaaaatttattagttccttcgatagaatcatgtatgttccggaagaggacctgttcgaatttgcactggactacaagaactttagccgtaccgatgctgattatataaagaaatggaaactctacagctatggcaaccgcatccgtatctttcgcaacccgaaaaaaaacaacgtgttcgactgggaagaggtatgtctgacttcagcgtacaaggagcttttcaataaatatggtatcaactaccagcagggtgatatccgtgcactgctttgcgagcaaagcgataaagcgttctattcaagcttcatggcgctgatgagcctgatgctgcaaatgcgcaacagcatcaccggtcgtaccgatgttgcatttctgatttctccagtaaaaaacagcgatggtatcttttacgacagccgtaattacgaggcccaagaaaatgcgattttgccgaagaacgctgacgcgaatggtgcatacaacattgcaaggaaagttctgtgggcaatcggccaatttaaaaaagcggaagatgagaagctggacaaagtgaaaattgcgattagcaacaaggagtggctggagtatgcgcagacctccgttaaatcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 43 - DNA, 5313 bp, synthetic construct
  source 1..5313: mol_type = other DNA
atgggaagtcatcaccaccaccaccattcaggtgagaacctgtacttccagagcatggctccgaaaaagaaacgtaaggtgggtatccacggcgttccggcgatgacaagtgaaaaaggaccctcaactggggacccaaccctgcgtcgtcgcattgaaagctgggagttcgacgttttttatgatccgcgtgagctggcgaaagagacgtgcctgttgtacgagatcaaatggggtatgagccgtaaaatctggagatccagcggtaaaaacaccaccaatcacgtggaagtgaattttatcaagaagttcaccagcgaacgtcgtttccacagctccatttcttgttctattacctggtttctgtcttggtcgccgtgttgggagtgctcccaggcaattcgtgaattccttagtcaacatccgggcgtgaccctggtcatctacgtcgcccgtttgttttggcatatggaccagcgcaatcgtcagggtttgcgcgatctggttaacagcggcgttacgatccaaattatgcgtgcgagcgagtattaccactgctggcgcaacttcgtgaactatccaccgggcgatgaagcacattggccgcaatatccgccgctgtggatgatgctgtacgcgctggaactccactgcattatcctgtcgctgccgccttgtctgaagatcagccgacgctggcagaaccacctcgcgtttttccgcctgcacttgcaaaactgccattaccagaccatcccgccgcatatcctgttagctaccggtctgattcacccgagcgttacttggcgttctggaagtgagacacccgggacttcagaatccgcgaccccggagtctggaatggataaaaagtattcaataggactagctattggcaccaacagcgttggttgggcagttatcaccgatgaatataaggtcccgagcaagaagtttaaggtcctgggcaacaccgatcgccacagcataaaaaagaaccttattggtgcgctgctgttcgattctggtgagactgccgaagcgacccgtctgaagagaaccgcgcgtcgtagatacacccggcgcaagaaccgtatctgctatctgcaagaaattttcagcaacgagatggctaaggtcgacgactccttctttcatcgcctggaggaaagctttctggttgaggaagacaaaaagcacgagcgtcatccgattttcggtaacattgttgatgaagttgcctaccacgaaaaataccccacaatttatcatctgcgtaaaaaactggtagactccaccgataaggcggatttacgccttatctatttggcgctggcgcatatgattaagttccgcggtcattttttgatcgagggcgacctgaatccggataacagcgacgttgacaaactgtttatccagctggtgcagacctataaccagttgttcgaagagaacccgattaatgcgtctggcgtggacgcgaaggcgatcctgagcgctcgtctgtccaagtcacgccgcctggagaacttgatcgcgcaactgccgggtgagaagaaaaacggcttgttcggcaatctgattgctctgtccctaggtttgaccccgaatttcaaatctaattttgatctggcggaagatgcgaaattacagttgagcaaggacacttacgatgacgatctggacaacctgctcgctcagatcggcgatcagtatgccgatcttttcctggccgcgaagaacctgagcgatgccatcttgctatcggacatcttacgtgttaacaccgaaatcaccaaagctccgctaagcgcgtccatgattaaacgttacgatgaacaccaccaggatctgacgctgctgaaagcgttggtgcgccagcaactgccggaaaaatacaaggaaattttcttcgaccaaagcaagaacggttatgcaggctatatcgacggtggtgcgtcgcaagaagaattttataaattcatcaaaccgattttggagaagatggatggtaccgaggagctgctggtaaaattgaatcgggaagacctcttgcgaaagcagcgtactttcgataatggtagcatcccgcaccagattcatctgggcgaacttcacgcaatcctacgtcgtcaagaggatttctacccgtttctgaaggataaccgtgaaaagatcgagaagattctgacctttcgtattccgtattatgtgggtccgctggcgagaggcaacagccgttttgcgtggatgacccgcaagtcggaagagactatcacgccgtggaatttcgaggaggtcgtggacaagggtgcatcagcgcaatcttttattgaacgtatgaccaattttgataaaaaccttccaaatgaaaaagtgttgccgaagcacagcttgctgtacgagtatttcaccgtttacaatgaactgacgaaagtcaagtacgttaccgaaggtatgcgtaagccagcctttctgtctggggagcaaaaaaaagccattgtggatctgctgttcaagaccaatcgcaaggttaccgtcaagcaactgaaagaggattacttcaaaaaaatcgaatgttttgactccgtggaaatcagcggcgttgaagaccgctttaatgcatctctgggcacctaccacgatctactcaagatcatcaaggataaggacttcctggacaacgaagaaaatgaggatatcttggaagatattgtgctgactttaacgttgttcgaggatcgtgagatgattgaagaacgcctcaaaacgtacgcacatctgtttgacgataaggtgatgaaacagctaaagcgccgtcgttacaccggttggggtcgtctgtctagaaagctgattaacggcattcgtgacaaacaatctggcaagaccatcctggacttcctcaaatccgacggcttcgcaaatcgtaatttcatgcaactgatccatgatgatagcctgacctttaaagaagatattcagaaggctcaagtgtccggccagggtgactcgctgcatgagcatatcgcgaatctggctggctcccctgcgataaaaaaaggtattctccaaacggtgaaagttgtggatgaactggttaaagtcatgggtcgtcataaaccggaaaacatcgtgattgagatggcacgtgagaaccagaccacccagaaaggtcaaaaaaacagtcgtgagcgtatgaaacgtatcgaagaggggattaaggagttaggcagccagattctgaaggagcacccggttgagaacacccagctgcaaaacgagaagctctacctgtattacctgcaaaacggccgtgacatgtatgttgaccaggagctggacatcaatcgtctcagcgactacgacgtggatgctatcgtgccgcagagctttctaaaagatgattccatcgacaataaagtgctcacccgtagcgacaagaaccgtggtaaaagcgacaacgtgccgagcgaggaagtagttaagaaaatgaaaaactactggcgtcagctattgaacgccaagcttatcacccagcgtaagtttgacaacttgacgaaggcagagcgtggcggcctgagcgagctagacaaggcgggttttattaaacgccagctggtggaaactcgccagattaccaaacacgttgcgcaaatcttggactcacgcatgaatacgaagtacgacgaaaacgataaattgatccgcgaggtgaaggtcataaccctgaaatctaaactggttagcgatttccggaaggactttcaattttataaagtccgggagattaacaactatcaccatgcacacgacgcttacttaaatgcggttgttggcacggccctgatcaagaagtacccgaaactggaaagcgagtttgtttatggtgactacaaggtgtatgacgttcgcaagatgattgcaaaatccgaacaagagattggtaaggcgacggctaagtattttttctacagcaatatcatgaacttcttcaagactgaaatcaccctggcgaacggtgaaattcgtaaacgtccgttgatcgaaaccaacggtgaaaccggtgaaatcgtttgggataaaggtagggattttgcgaccgttcgtaaggtcctgagcatgccgcaggttaacatcgtgaaaaagaccgaagtgcagaccggcggtttctccaaggagagtatcttaccgaagcgcaacagtgacaagctgatcgcgcgtaaaaaagactgggatccgaaaaaatacggcggctttgactccccgaccgtcgcgtacagcgtactggtcgtggctaaagtggagaaaggcaaatcgaaaaagctgaaatccgttaaagagttgctgggtattacgattatggaacgttcgagcttcgagaaaaatccgatcgacttcctggaggccaaaggctacaaggaagttaaaaaagacctgatcattaaactgccgaagtacagcctgttcgagctcgagaacggtcgcaaacgcatgctggcgagcgcgggtgagctccaaaaaggcaacgaattggctctgccgagcaagtacgtgaacttcctgtatctggcctcccattatgaaaaactgaagggtagcccggaagataacgaacaaaagcagctgttcgtggaacaacataaacactatctggacgagattattgagcaaatcagcgagttcagcaaacgcgtgattctggcggatgctaatctggacaaggttctttcagcctataataaacaccgtgataagccgatccgtgaacaggcagaaaatattattcacctgttcaccctgacgaacctgggtgcgccagcagcgtttaaatacttcgacacaacgatcgaccggaagcgttataccagcaccaaagaggttcttgacgccaccttgattcaccaaagcatcacgggtctgtatgaaacccgtattgacttgagccagttgggcggagattcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 44 - DNA, 3474 bp, synthetic construct
  source 1..3474: mol_type = other DNA
atgggaagtcatcaccaccaccaccattcaggtgagaacctgtacttccagagcatggctccgaaaaagaaacgtaaggtgggtatccacggcgttccggcgatgacaagtgaaaaaggaccctcaactggggacccaaccctgcgtcgtcgcattgaaagctgggagttcgacgttttttatgatccgcgtgagctggcgaaagagacgtgcctgttgtacgagatcaaatggggtatgagccgtaaaatctggagatccagcggtaaaaacaccaccaatcacgtggaagtgaattttatcaagaagttcaccagcgaacgtcgtttccacagctccatttcttgttctattacctggtttctgtcttggtcgccgtgttgggagtgctcccaggcaattcgtgaattccttagtcaacatccgggcgtgaccctggtcatctacgtcgcccgtttgttttggcatatggaccagcgcaatcgtcagggtttgcgcgatctggttaacagcggcgttacgatccaaattatgcgtgcgagcgagtattaccactgctggcgcaacttcgtgaactatccaccgggcgatgaagcacattggccgcaatatccgccgctgtggatgatgctgtacgcgctggaactccactgcattatcctgtcgctgccgccttgtctgaagatcagccgacgctggcagaaccacctcgcgtttttccgcctgcacttgcaaaactgccattaccagaccatcccgccgcatatcctgttagctaccggtctgattcacccgagcgttacttggcgttctggaagtgagacacccgggacttcagaatccgcgaccccggagtctggaatgccaaaacccgctgtagagtcagaattttcaaaagttttgaaaaagcactttccgggagaacgtttccgctcgagctatatgaaaagaggcggtaagatcctggctgcccaaggcgaagaggcagttgttgcgtatctacagggcaagtccgaggaggagcctccgaacttccagccaccggcgaagtgccacgtggtgaccaaatcccgtgacttcgcggaatggccgatcatgaaggcgtcggaagcgattcaacgttatatctatgcactgtcgaccaccgaacgcgcggcctgtaaaccgggcaaaagtagtgagagccatgcagcgtggttcgccgctaccggtgtcagcaaccacggctacagccacgttcagggtctgaacctgatttttgatcataccttgggccgttacgatggtgttctgaaaaaggtccaactgcgcaacgaaaaggcgagagcgcgtttagagagcattaacgcgtctagagcagatgaaggtttaccggagattaaagccgaggaggaagaagtggctacgaacgaaaccggccacctgttgcaaccgccgggtatcaatccgagcttttacgtgtatcagaccattagcccgcaggcataccgtccgcgtgacgagatcgtgctgccgccggagtacgcaggttacgtacgtgacccgaatgctccgattccgctgggtgtggtgcgtaaccgttgcgatatccaaaaagggtgcccgggctacatcccggaatggcagcgtgaagcaggtaccgcgatcagcccgaaaacaggcaaggccgtaactgtgccgggcctgtctccgaagaaaaacaaacgtatgcgtaggtactggcgctctgagaaggagaaagctcaagatgcgctgttggttaccgtacgcattggtaccgattgggttgtcatcgacgtgcgcggtctgttgcgcaacgcccgttggcgcaccatcgctcccaaggacatcagcctgaatgcgctcctcgatttgtttaccggtgatccggtgatcgatgtgcgtcgcaatattgttacctttacgtacaccctcgacgcctgtggtacctacgcccgtaaatggaccttgaaaggcaaacagacgaaagcgaccctggacaaactgacggccactcagacggttgcgttggttgcgattgcgcttggtcagaccaacccgatttccgctggcattagccgtgttacacaagaaaatggtgcactgcaatgcgaaccgctggaccggttcaccctgccggatgacctgttgaaggacatcagcgcatatcgcattgcttgggacagaaacgaagaggaactacgcgctcgcagcgtcgaggccctgccggaggcgcaacaagcggaggtccgtgcgctggacggggtgtccaaagaaaccgcgcgtacccaactgtgcgcagatttcggcctggacccgaaacgtctgccgtgggataaaatgagctccaatactacctttatcagcgaagccttgctgtctaacagcgtgagccgtgatcaggtttttttcactccggctccgaagaagggtgctaagaaaaaagcgcctgttgaagttatgcgtaaagaccgcacttgggcacgtgcgtacaagccgcgtctgagcgtcgaagctcagaaactgaagaacgaagccctgtgggcacttaagcgcacctccccggagtatttgaagctgagccgtcgcaaagaggagctgtgtcgtcgtagcatcaactatgttatcgaaaagacccgtcgccgtacgcaatgtcagattgtgatcccggtgatcgaggatctgaatgttcgcttctttcatggttctggtaagagactgcccggttgggataatttcttcaccgctaaaaaagagaaccgttggttcatccagggcctgcacaaagcgtttagcgatctgcgtacgcaccgttccttctatgtttttgaagttcgtccggagcgcacctctattacctgtccaaagtgcggtcattgcgaggtgggtaatcgtgacggtgaggcgtttcagtgcctgagctgcggcaagacgtgcaacgcagatcttgacgtcgcgacccataatctgacccaagtggcgttgaccggtaagaccatgccgaagcgcgaggagccgcgtgacgctcagggtacagcgccagcgcgcaagactaagaaggcgagcaaatcaaaggcgccaccggcggagcgcgaagaccagaccccggcgcaagagccaagccagaccagttctggaggtagcgggggtagcggtggtagcactaacttaagtgatattatcgagaaagaaaccgggaaacagctggtgatccaagaaagcattctgatgctgcccgaagaagttgaagaagtgattggtaacaagcctgagtcggacatcttagtgcacaccgcgtacgatgaaagcacggatgagaatgtgatgctgctcacctctgatgcccccgaatacaagccgtgggctctggtgatccaggactctaacggagaaaacaaaattaagatgctctctggcggctccatggcgcctaaaaaaaaacgcaaagtggggattcacggggttccggcgtaa

SEQ ID NO: 45 - DNA, 4890 bp, synthetic construct
  source 1..4890: mol_type = other DNA
atgggaagtcatcaccaccaccaccattcaggtgagaacctgtacttccagagcatggctccgaaaaagaaacgtaaggtgggtatccacggcgttccggcgatgacaagtgaaaaaggaccctcaactggggacccaaccctgcgtcgtcgcattgaaagctgggagttcgacgttttttatgatccgcgtgagctggcgaaagagacgtgcctgttgtacgagatcaaatggggtatgagccgtaaaatctggagatccagcggtaaaaacaccaccaatcacgtggaagtgaattttatcaagaagttcaccagcgaacgtcgtttccacagctccatttcttgttctattacctggtttctgtcttggtcgccgtgttgggagtgctcccaggcaattcgtgaattccttagtcaacatccgggcgtgaccctggtcatctacgtcgcccgtttgttttggcatatggaccagcgcaatcgtcagggtttgcgcgatctggttaacagcggcgttacgatccaaattatgcgtgcgagcgagtattaccactgctggcgcaacttcgtgaactatccaccgggcgatgaagcacattggccgcaatatccgccgctgtggatgatgctgtacgcgctggaactccactgcattatcctgtcgctgccgccttgtctgaagatcagccgacgctggcagaaccacctcgcgtttttccgcctgcacttgcaaaactgccattaccagaccatcccgccgcatatcctgttagctaccggtctgattcacccgagcgttacttggcgttctggaagtgagacacccgggacttcagaatccgcgaccccggagtctggaatgtcaaaactagaaaagtttacaaattgttactccctgtcgaaaaccctgagattcaaagcgatcccggtgggtaaaactcaagagaacattgataacaaacgtctgctggttgaggacgaaaagcgcgcggaggattacaaaggcgttaaaaagctgttggaccgttattacctgtcctttattaatgatgttctgcacagcattaaattaaagaacctaaacaattacatctccctcttccgtaaaaagacccgcaccgaaaaggaaaataaagaactggagaaccttgagattaatctgcgtaaagaaatcgctaaagcgtttaaaggcaacgagggttataaatccttatttaaaaaagacatcatcgaaacgattctcccagaattcctggacgacaaagacgagatcgcgctggttaattccttcaacggctttaccaccgcattcaccggcttttttgataaccgcgaaaacatgttctctgaagaggccaaaagcacctcaattgcgtttcgctgcattaacgaaaatctgacgcgttatatctctaacatggatatcttcgagaaggttgacgctatctttgataaacatgaagtccaggagatcaaggagaagatcctgaacagcgactacgacgtggaggatttcttcgagggtgagttcttcaattttgttttgacccaagaaggcatcgatgtctacaatgcgataattggtggttttgttaccgagagcggggagaaaattaagggtctgaacgaatatatcaacttgtacaaccagaaaaccaaacaaaaactgccgaagtttaagccgctgtacaagcaagtcttgtcggatcgtgaatcgctgagcttttacggcgaaggctacaccagcgatgaggaggtgttggaggtgttccgcaatacgttgaataagaactctgaaattttttccagcattaagaagctggaaaaactattcaagaacttcgacgagtatagcagcgcaggtatctttgtgaaaaacggtccggctattagcaccatctcgaaagacattttcggcgaatggaatgttattcgtgataagtggaatgccgaatacgacgacattcatctgaagaaaaaggcggttgtaactgaaaagtacgaggacgatcgtcgtaagtcgttcaaaaagatcggctccttctctctggagcaactgcaagagtacgccgatgccgatttgtcagtcgtggagaagctgaaagagatcatcattcaaaaagttgacgaaatctataaggtctatggtagcagtgaaaaactgtttgacgcggacttcgtgctggagaaaagcctgaagaaaaatgacgccgtggtggcaatcatgaaagatcttctggatagcgtcaagtctttcgaaaactacatcaaagcgtttttcggtgaaggcaaggaaacgaatcgtgacgagagcttctatggtgatttcgtcctagcctatgatatcctgcttaaggtggatcacatctatgatgccatccgcaactatgttacccagaaaccgtactcaaaggacaagttcaagttatacttccagaatccgcagtttatgggtggctgggataaagataaggaaaccgactatcgcgccaccattttacgttacggtagtaagtactatctggcaatcatggataaaaaatatgcgaaatgcctgcaaaagatcgacaaggacgatgttaacggcaattatgagaagatcaactacaaactgctgccgggcccaaataagatgctgccgaaggtgtttttcagcaaaaaatggatggcgtattacaatccgagcgaggacatccaaaagatctacaaaaacggtacgtttaagaagggtgatatgttcaacctcaacgattgtcataaacttattgacttcttcaaggatagcatatctcgttatccgaagtggagcaacgcgtatgactttaactttagcgaaaccgagaaatacaaagatatcgcgggtttctatcgtgaagttgaagaacagggttacaaagtgtccttcgagtccgcgagcaagaaggaggtagacaagctggtcgaggagggtaagttgtacatgtttcagatttacaataaggacttttccgacaagtcccatggtacaccgaatttgcacaccatgtatttcaagcttctgttcgacgagaacaaccacggccagatccgtctgagcggtggtgcagagctgttcatgcgtagagctagcctgaagaaagaagagttggtcgttcatccggctaattcgccgatcgcgaacaaaaatccagataaccctaaaaagactacgaccctgagctatgatgtgtataaagacaaacgctttagcgaagaccagtatgagttacacattccgatcgctatcaacaagtgcccgaagaatattttcaagatcaacaccgaagtgcgtgttcttttgaagcacgatgacaacccgtatgtgattggtattgctcgtggtgaacgtaatctgctgtacattgtggttgtggacggcaaaggaaacatcgtggaacagtacagcctgaacgaaatcattaacaatttcaacggcattcgcattaagacggattaccattctttgctggacaaaaaggagaaagagcgtttcgaggcccgtcagaactggacctccatcgagaacattaaggagctgaaggcgggttacatcagccaggtggtgcacaaaatctgcgaactggtggagaagtacgacgcggtgattgctttggcggatttgaactccggttttaagaacagccgcgttaaagtggaaaagcaggtttatcagaaattcgaaaaaatgctgattgataaattgaattacatggttgacaaaaagtctaacccgtgtgcaaccggcggtgcgctgaagggctaccaaatcaccaataagtttgaaagctttaagagcatgagcacgcaaaatggcttcattttttacattccggcgtggctgaccagcaagatcgatccgtcaaccggctttgttaatttactgaagacgaagtatacctctattgccgactctaaaaaatttattagttccttcgatagaatcatgtatgttccggaagaggacctgttcgaatttgcactggactacaagaactttagccgtaccgatgctgattatataaagaaatggaaactctacagctatggcaaccgcatccgtatctttcgcaacccgaaaaaaaacaacgtgttcgactgggaagaggtatgtctgacttcagcgtacaaggagcttttcaataaatatggtatcaactaccagcagggtgatatccgtgcactgctttgcgagcaaagcgataaagcgttctattcaagcttcatggcgctgatgagcctgatgctgcaaatgcgcaacagcatcaccggtcgtaccgatgttgcatttctgatttctccagtaaaaaacagcgatggtatcttttacgacagccgtaattacgaggcccaagaaaatgcgattttgccgaagaacgctgacgcgaatggtgcatacaacattgcaaggaaagttctgtgggcaatcggccaatttaaaaaagcggaagatgagaagctggacaaagtgaaaattgcgattagcaacaaggagtggctggagtatgcgcagacctccgttaaatcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 46 - DNA, 5292 bp, synthetic construct
  source 1..5292: mol_type = other DNA
atgggaagtcatcaccaccaccaccattcaggtgagaacctgtacttccagagcatggctccgaaaaagaaacgtaaggtgggtatccacggcgttccggcgatgagttcagaaacaggacccgttgctgtagacccgaccctgcgtcgccgcattgagccgcacgagttcgaagtcttttttgatccgcgtgaactgcgcaaagaaacctgtctgctgtatgaaattaactggggtggccgtcacagcatttggcgtcacactagtcaaaacaccaataaacacgtggaagttaacttcatcgagaaatttaccaccgagcgctacttctgcccgaatactcgttgttctattacgtggtttctctcttggtcgccgtgcggcgagtgcagccgtgcgattaccgagttcctgagccgttacccgcacgttaccctgtttatctatatcgcgcgtctgtaccatcatgcggacccgcgtaaccgtcaaggtctgcgtgatctgatcagcagcggtgttacgatccagattatgacggagcaggagtccggctattgctggcgcaacttcgtgaactatagcccatccaatgaagctcattggccgcgctacccgcacctgtgggtacgtttgtacgtgctggaactctactgcatcatccttggcctgccaccgtgcttgaatattttgagacgcaagcagccgcaactgaccttcttcaccatcgccctgcaaagctgtcattatcagcgtttgccgcctcacatcttatgggcaaccggtttgaagtctggaagtgagacacccgggacttcagaatccgcgaccccggagtctggaatggataaaaagtattcaataggactagctattggcaccaacagcgttggttgggcagttatcaccgatgaatataaggtcccgagcaagaagtttaaggtcctgggcaacaccgatcgccacagcataaaaaagaaccttattggtgcgctgctgttcgattctggtgagactgccgaagcgacccgtctgaagagaaccgcgcgtcgtagatacacccggcgcaagaaccgtatctgctatctgcaagaaattttcagcaacgagatggctaaggtcgacgactccttctttcatcgcctggaggaaagctttctggttgaggaagacaaaaagcacgagcgtcatccgattttcggtaacattgttgatgaagttgcctaccacgaaaaataccccacaatttatcatctgcgtaaaaaactggtagactccaccgataaggcggatttacgccttatctatttggcgctggcgcatatgattaagttccgcggtcattttttgatcgagggcgacctgaatccggataacagcgacgttgacaaactgtttatccagctggtgcagacctataaccagttgttcgaagagaacccgattaatgcgtctggcgtggacgcgaaggcgatcctgagcgctcgtctgtccaagtcacgccgcctggagaacttgatcgcgcaactgccgggtgagaagaaaaacggcttgttcggcaatctgattgctctgtccctaggtttgaccccgaatttcaaatctaattttgatctggcggaagatgcgaaattacagttgagcaaggacacttacgatgacgatctggacaacctgctcgctcagatcggcgatcagtatgccgatcttttcctggccgcgaagaacctgagcgatgccatcttgctatcggacatcttacgtgttaacaccgaaatcaccaaagctccgctaagcgcgtccatgattaaacgttacgatgaacaccaccaggatctgacgctgctgaaagcgttggtgcgccagcaactgccggaaaaatacaaggaaattttcttcgaccaaagcaagaacggttatgcaggctatatcgacggtggtgcgtcgcaagaagaattttataaattcatcaaaccgattttggagaagatggatggtaccgaggagctgctggtaaaattgaatcgggaagacctcttgcgaaagcagcgtactttcgataatggtagcatcccgcaccagattcatctgggcgaacttcacgcaatcctacgtcgtcaagaggatttctacccgtttctgaaggataaccgtgaaaagatcgagaagattctgacctttcgtattccgtattatgtgggtccgctggcgagaggcaacagccgttttgcgtggatgacccgcaagtcggaagagactatcacgccgtggaatttcgaggaggtcgtggacaagggtgcatcagcgcaatcttttattgaacgtatgaccaattttgataaaaaccttccaaatgaaaaagtgttgccgaagcacagcttgctgtacgagtatttcaccgtttacaatgaactgacgaaagtcaagtacgttaccgaaggtatgcgtaagccagcctttctgtctggggagcaaaaaaaagccattgtggatctgctgttcaagaccaatcgcaaggttaccgtcaagcaactgaaagaggattacttcaaaaaaatcgaatgttttgactccgtggaaatcagcggcgttgaagaccgctttaatgcatctctgggcacctaccacgatctactcaagatcatcaaggataaggacttcctggacaacgaagaaaatgaggatatcttggaagatattgtgctgactttaacgttgttcgaggatcgtgagatgattgaagaacgcctcaaaacgtacgcacatctgtttgacgataaggtgatgaaacagctaaagcgccgtcgttacaccggttggggtcgtctgtctagaaagctgattaacggcattcgtgacaaacaatctggcaagaccatcctggacttcctcaaatccgacggcttcgcaaatcgtaatttcatgcaactgatccatgatgatagcctgacctttaaagaagatattcagaaggctcaagtgtccggccagggtgactcgctgcatgagcatatcgcgaatctggctggctcccctgcgataaaaaaaggtattctccaaacggtgaaagttgtggatgaactggttaaagtcatgggtcgtcataaaccggaaaacatcgtgattgagatggcacgtgagaaccagaccacccagaaaggtcaaaaaaacagtcgtgagcgtatgaaacgtatcgaagaggggattaaggagttaggcagccagattctgaaggagcacccggttgagaacacccagctgcaaaacgagaagctctacctgtattacctgcaaaacggccgtgacatgtatgttgaccaggagctggacatcaatcgtctcagcgactacgacgtggatgctatcgtgccgcagagctttctaaaagatgattccatcgacaataaagtgctcacccgtagcgacaagaaccgtggtaaaagcgacaacgtgccgagcgaggaagtagttaagaaaatgaaaaactactggcgtcagctattgaacgccaagcttatcacccagcgtaagtttgacaacttgacgaaggcagagcgtggcggcctgagcgagctagacaaggcgggttttattaaacgccagctggtggaaactcgccagattaccaaacacgttgcgcaaatcttggactcacgcatgaatacgaagtacgacgaaaacgataaattgatccgcgaggtgaaggtcataaccctgaaatctaaactggttagcgatttccggaaggactttcaattttataaagtccgggagattaacaactatcaccatgcacacgacgcttacttaaatgcggttgttggcacggccctgatcaagaagtacccgaaactggaaagcgagtttgtttatggtgactacaaggtgtatgacgttcgcaagatgattgcaaaatccgaacaagagattggtaaggcgacggctaagtattttttctacagcaatatcatgaacttcttcaagactgaaatcaccctggcgaacggtgaaattcgtaaacgtccgttgatcgaaaccaacggtgaaaccggtgaaatcgtttgggataaaggtagggattttgcgaccgttcgtaaggtcctgagcatgccgcaggttaacatcgtgaaaaagaccgaagtgcagaccggcggtttctccaaggagagtatcttaccgaagcgcaacagtgacaagctgatcgcgcgtaaaaaagactgggatccgaaaaaatacggcggctttgactccccgaccgtcgcgtacagcgtactggtcgtggctaaagtggagaaaggcaaatcgaaaaagctgaaatccgttaaagagttgctgggtattacgattatggaacgttcgagcttcgagaaaaatccgatcgacttcctggaggccaaaggctacaaggaagttaaaaaagacctgatcattaaactgccgaagtacagcctgttcgagctcgagaacggtcgcaaacgcatgctggcgagcgcgggtgagctccaaaaaggcaacgaattggctctgccgagcaagtacgtgaacttcctgtatctggcctcccattatgaaaaactgaagggtagcccggaagataacgaacaaaagcagctgttcgtggaacaacataaacactatctggacgagattattgagcaaatcagcgagttcagcaaacgcgtgattctggcggatgctaatctggacaaggttctttcagcctataataaacaccgtgataagccgatccgtgaacaggcagaaaatattattcacctgttcaccctgacgaacctgggtgcgccagcagcgtttaaatacttcgacacaacgatcgaccggaagcgttataccagcaccaaagaggttcttgacgccaccttgattcaccaaagcatcacgggtctgtatgaaacccgtattgacttgagccagttgggcggagattcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 47 - DNA, 3453 bp, synthetic construct
  source 1..3453: mol_type = other DNA
atgggaagtcatcaccaccaccaccattcaggtgagaacctgtacttccagagcatggctccgaaaaagaaacgtaaggtgggtatccacggcgttccggcgatgagttcagaaacaggacccgttgctgtagacccgaccctgcgtcgccgcattgagccgcacgagttcgaagtcttttttgatccgcgtgaactgcgcaaagaaacctgtctgctgtatgaaattaactggggtggccgtcacagcatttggcgtcacactagtcaaaacaccaataaacacgtggaagttaacttcatcgagaaatttaccaccgagcgctacttctgcccgaatactcgttgttctattacgtggtttctctcttggtcgccgtgcggcgagtgcagccgtgcgattaccgagttcctgagccgttacccgcacgttaccctgtttatctatatcgcgcgtctgtaccatcatgcggacccgcgtaaccgtcaaggtctgcgtgatctgatcagcagcggtgttacgatccagattatgacggagcaggagtccggctattgctggcgcaacttcgtgaactatagcccatccaatgaagctcattggccgcgctacccgcacctgtgggtacgtttgtacgtgctggaactctactgcatcatccttggcctgccaccgtgcttgaatattttgagacgcaagcagccgcaactgaccttcttcaccatcgccctgcaaagctgtcattatcagcgtttgccgcctcacatcttatgggcaaccggtttgaagtctggaagtgagacacccgggacttcagaatccgcgaccccggagtctggaatgccaaaacccgctgtagagtcagaattttcaaaagttttgaaaaagcactttccgggagaacgtttccgctcgagctatatgaaaagaggcggtaagatcctggctgcccaaggcgaagaggcagttgttgcgtatctacagggcaagtccgaggaggagcctccgaacttccagccaccggcgaagtgccacgtggtgaccaaatcccgtgacttcgcggaatggccgatcatgaaggcgtcggaagcgattcaacgttatatctatgcactgtcgaccaccgaacgcgcggcctgtaaaccgggcaaaagtagtgagagccatgcagcgtggttcgccgctaccggtgtcagcaaccacggctacagccacgttcagggtctgaacctgatttttgatcataccttgggccgttacgatggtgttctgaaaaaggtccaactgcgcaacgaaaaggcgagagcgcgtttagagagcattaacgcgtctagagcagatgaaggtttaccggagattaaagccgaggaggaagaagtggctacgaacgaaaccggccacctgttgcaaccgccgggtatcaatccgagcttttacgtgtatcagaccattagcccgcaggcataccgtccgcgtgacgagatcgtgctgccgccggagtacgcaggttacgtacgtgacccgaatgctccgattccgctgggtgtggtgcgtaaccgttgcgatatccaaaaagggtgcccgggctacatcccggaatggcagcgtgaagcaggtaccgcgatcagcccgaaaacaggcaaggccgtaactgtgccgggcctgtctccgaagaaaaacaaacgtatgcgtaggtactggcgctctgagaaggagaaagctcaagatgcgctgttggttaccgtacgcattggtaccgattgggttgtcatcgacgtgcgcggtctgttgcgcaacgcccgttggcgcaccatcgctcccaaggacatcagcctgaatgcgctcctcgatttgtttaccggtgatccggtgatcgatgtgcgtcgcaatattgttacctttacgtacaccctcgacgcctgtggtacctacgcccgtaaatggaccttgaaaggcaaacagacgaaagcgaccctggacaaactgacggccactcagacggttgcgttggttgcgattgcgcttggtcagaccaacccgatttccgctggcattagccgtgttacacaagaaaatggtgcactgcaatgcgaaccgctggaccggttcaccctgccggatgacctgttgaaggacatcagcgcatatcgcattgcttgggacagaaacgaagaggaactacgcgctcgcagcgtcgaggccctgccggaggcgcaacaagcggaggtccgtgcgctggacggggtgtccaaagaaaccgcgcgtacccaactgtgcgcagatttcggcctggacccgaaacgtctgccgtgggataaaatgagctccaatactacctttatcagcgaagccttgctgtctaacagcgtgagccgtgatcaggtttttttcactccggctccgaagaagggtgctaagaaaaaagcgcctgttgaagttatgcgtaaagaccgcacttgggcacgtgcgtacaagccgcgtctgagcgtcgaagctcagaaactgaagaacgaagccctgtgggcacttaagcgcacctccccggagtatttgaagctgagccgtcgcaaagaggagctgtgtcgtcgtagcatcaactatgttatcgaaaagacccgtcgccgtacgcaatgtcagattgtgatcccggtgatcgaggatctgaatgttcgcttctttcatggttctggtaagagactgcccggttgggataatttcttcaccgctaaaaaagagaaccgttggttcatccagggcctgcacaaagcgtttagcgatctgcgtacgcaccgttccttctatgtttttgaagttcgtccggagcgcacctctattacctgtccaaagtgcggtcattgcgaggtgggtaatcgtgacggtgaggcgtttcagtgcctgagctgcggcaagacgtgcaacgcagatcttgacgtcgcgacccataatctgacccaagtggcgttgaccggtaagaccatgccgaagcgcgaggagccgcgtgacgctcagggtacagcgccagcgcgcaagactaagaaggcgagcaaatcaaaggcgccaccggcggagcgcgaagaccagaccccggcgcaagagccaagccagaccagttctggaggtagcgggggtagcggtggtagcactaacttaagtgatattatcgagaaagaaaccgggaaacagctggtgatccaagaaagcattctgatgctgcccgaagaagttgaagaagtgattggtaacaagcctgagtcggacatcttagtgcacaccgcgtacgatgaaagcacggatgagaatgtgatgctgctcacctctgatgcccccgaatacaagccgtgggctctggtgatccaggactctaacggagaaaacaaaattaagatgctctctggcggctccatggcgcctaaaaaaaaacgcaaagtggggattcacggggttccggcgtaa

SEQ ID NO: 48 - DNA, 4869 bp, synthetic construct
  source 1..4869: mol_type = other DNA
atgggaagtcatcaccaccaccaccattcaggtgagaacctgtacttccagagcatggctccgaaaaagaaacgtaaggtgggtatccacggcgttccggcgatgagttcagaaacaggacccgttgctgtagacccgaccctgcgtcgccgcattgagccgcacgagttcgaagtcttttttgatccgcgtgaactgcgcaaagaaacctgtctgctgtatgaaattaactggggtggccgtcacagcatttggcgtcacactagtcaaaacaccaataaacacgtggaagttaacttcatcgagaaatttaccaccgagcgctacttctgcccgaatactcgttgttctattacgtggtttctctcttggtcgccgtgcggcgagtgcagccgtgcgattaccgagttcctgagccgttacccgcacgttaccctgtttatctatatcgcgcgtctgtaccatcatgcggacccgcgtaaccgtcaaggtctgcgtgatctgatcagcagcggtgttacgatccagattatgacggagcaggagtccggctattgctggcgcaacttcgtgaactatagcccatccaatgaagctcattggccgcgctacccgcacctgtgggtacgtttgtacgtgctggaactctactgcatcatccttggcctgccaccgtgcttgaatattttgagacgcaagcagccgcaactgaccttcttcaccatcgccctgcaaagctgtcattatcagcgtttgccgcctcacatcttatgggcaaccggtttgaagtctggaagtgagacacccgggacttcagaatccgcgaccccggagtctggaatgtcaaaactagaaaagtttacaaattgttactccctgtcgaaaaccctgagattcaaagcgatcccggtgggtaaaactcaagagaacattgataacaaacgtctgctggttgaggacgaaaagcgcgcggaggattacaaaggcgttaaaaagctgttggaccgttattacctgtcctttattaatgatgttctgcacagcattaaattaaagaacctaaacaattacatctccctcttccgtaaaaagacccgcaccgaaaaggaaaataaagaactggagaaccttgagattaatctgcgtaaagaaatcgctaaagcgtttaaaggcaacgagggttataaatccttatttaaaaaagacatcatcgaaacgattctcccagaattcctggacgacaaagacgagatcgcgctggttaattccttcaacggctttaccaccgcattcaccggcttttttgataaccgcgaaaacatgttctctgaagaggccaaaagcacctcaattgcgtttcgctgcattaacgaaaatctgacgcgttatatctctaacatggatatcttcgagaaggttgacgctatctttgataaacatgaagtccaggagatcaaggagaagatcctgaacagcgactacgacgtggaggatttcttcgagggtgagttcttcaattttgttttgacccaagaaggcatcgatgtctacaatgcgataattggtggttttgttaccgagagcggggagaaaattaagggtctgaacgaatatatcaacttgtacaaccagaaaaccaaacaaaaactgccgaagtttaagccgctgtacaagcaagtcttgtcggatcgtgaatcgctgagcttttacggcgaaggctacaccagcgatgaggaggtgttggaggtgttccgcaatacgttgaataagaactctgaaattttttccagcattaagaagctggaaaaactattcaagaacttcgacgagtatagcagcgcaggtatctttgtgaaaaacggtccggctattagcaccatctcgaaagacattttcggcgaatggaatgttattcgtgataagtggaatgccgaatacgacgacattcatctgaagaaaaaggcggttgtaactgaaaagtacgaggacgatcgtcgtaagtcgttcaaaaagatcggctccttctctctggagcaactgcaagagtacgccgatgccgatttgtcagtcgtggagaagctgaaagagatcatcattcaaaaagttgacgaaatctataaggtctatggtagcagtgaaaaactgtttgacgcggacttcgtgctggagaaaagcctgaagaaaaatgacgccgtggtggcaatcatgaaagatcttctggatagcgtcaagtctttcgaaaactacatcaaagcgtttttcggtgaaggcaaggaaacgaatcgtgacgagagcttctatggtgatttcgtcctagcctatgatatcctgcttaaggtggatcacatctatgatgccatccgcaactatgttacccagaaaccgtactcaaaggacaagttcaagttatacttccagaatccgcagtttatgggtggctgggataaagataaggaaaccgactatcgcgccaccattttacgttacggtagtaagtactatctggcaatcatggataaaaaatatgcgaaatgcctgcaaaagatcgacaaggacgatgttaacggcaattatgagaagatcaactacaaactgctgccgggcccaaataagatgctgccgaaggtgtttttcagcaaaaaatggatggcgtattacaatccgagcgaggacatccaaaagatctacaaaaacggtacgtttaagaagggtgatatgttcaacctcaacgattgtcataaacttattgacttcttcaaggatagcatatctcgttatccgaagtggagcaacgcgtatgactttaactttagcgaaaccgagaaatacaaagatatcgcgggtttctatcgtgaagttgaagaacagggttacaaagtgtccttcgagtccgcgagcaagaaggaggtagacaagctggtcgaggagggtaagttgtacatgtttcagatttacaataaggacttttccgacaagtcccatggtacaccgaatttgcacaccatgtatttcaagcttctgttcgacgagaacaaccacggccagatccgtctgagcggtggtgcagagctgttcatgcgtagagctagcctgaagaaagaagagttggtcgttcatccggctaattcgccgatcgcgaacaaaaatccagataaccctaaaaagactacgaccctgagctatgatgtgtataaagacaaacgctttagcgaagaccagtatgagttacacattccgatcgctatcaacaagtgcccgaagaatattttcaagatcaacaccgaagtgcgtgttcttttgaagcacgatgacaacccgtatgtgattggtattgctcgtggtgaacgtaatctgctgtacattgtggttgtggacggcaaaggaaacatcgtggaacagtacagcctgaacgaaatcattaacaatttcaacggcattcgcattaagacggattaccattctttgctggacaaaaaggagaaagagcgtttcgaggcccgtcagaactggacctccatcgagaacattaaggagctgaaggcgggttacatcagccaggtggtgcacaaaatctgcgaactggtggagaagtacgacgcggtgattgctttggcggatttgaactccggttttaagaacagccgcgttaaagtggaaaagcaggtttatcagaaattcgaaaaaatgctgattgataaattgaattacatggttgacaaaaagtctaacccgtgtgcaaccggcggtgcgctgaagggctaccaaatcaccaataagtttgaaagctttaagagcatgagcacgcaaaatggcttcattttttacattccggcgtggctgaccagcaagatcgatccgtcaaccggctttgttaatttactgaagacgaagtatacctctattgccgactctaaaaaatttattagttccttcgatagaatcatgtatgttccggaagaggacctgttcgaatttgcactggactacaagaactttagccgtaccgatgctgattatataaagaaatggaaactctacagctatggcaaccgcatccgtatctttcgcaacccgaaaaaaaacaacgtgttcgactgggaagaggtatgtctgacttcagcgtacaaggagcttttcaataaatatggtatcaactaccagcagggtgatatccgtgcactgctttgcgagcaaagcgataaagcgttctattcaagcttcatggcgctgatgagcctgatgctgcaaatgcgcaacagcatcaccggtcgtaccgatgttgcatttctgatttctccagtaaaaaacagcgatggtatcttttacgacagccgtaattacgaggcccaagaaaatgcgattttgccgaagaacgctgacgcgaatggtgcatacaacattgcaaggaaagttctgtgggcaatcggccaatttaaaaaagcggaagatgagaagctggacaaagtgaaaattgcgattagcaacaaggagtggctggagtatgcgcagacctccgttaaatcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 49 - DNA, 5292 bp, synthetic construct
  source 1..5292: mol_type = other DNA
atgggaagtcatcaccaccaccaccattcaggtgagaacctgtacttccagagcatggctccgaaaaagaaacgtaaggtgggtatccacggcgttccggcgatgagttcagaaacaggacccgttgctgtagacccgaccttgcgcagacgtattgagccgcacgaattcgaggtgtttttcgacccgagagaattgcgcaaagaaacctgtctgctgtacgagatcaactggggtggtcgtcatagcatctggcgtcacacctctcagaatacgaacaaacacgtggaagtcaatttcatcgagaagttcacgaccgagcgctacttctgtccgaacacccgttgttcgatcacgtggtttctgagctattctccgtgcggcgaatgcagccgtgcaattaccgagttcctgtcacgttacccgcacgtaactctgtttatctacattgcgcgtctctaccatcatgctgatccggaaaaccgtcaaggtttacgcgatctgattagctccggcgtgaccatccagattatgactgagcaggagagcggctattgctggcgcaactttgttaactattccccgtccaatgaggcgcattggcctcgttatccgcacctgtgggttcgtctatacgttctggaattgtactgcattatcctgggcctgccaccatgcttgaatattctgcgtcgcaagcagccgcaattgacctttttcaccatcgccctgcaaagctgccattatcagcgccttccgccgcacatcctgtgggcgaccggtctgaaatctggaagtgagacacccgggacttcagaatccgcgaccccggagtctggaatggataaaaagtattcaataggactagctattggcaccaacagcgttggttgggcagttatcaccgatgaatataaggtcccgagcaagaagtttaaggtcctgggcaacaccgatcgccacagcataaaaaagaaccttattggtgcgctgctgttcgattctggtgagactgccgaagcgacccgtctgaagagaaccgcgcgtcgtagatacacccggcgcaagaaccgtatctgctatctgcaagaaattttcagcaacgagatggctaaggtcgacgactccttctttcatcgcctggaggaaagctttctggttgaggaagacaaaaagcacgagcgtcatccgattttcggtaacattgttgatgaagttgcctaccacgaaaaataccccacaatttatcatctgcgtaaaaaactggtagactccaccgataaggcggatttacgccttatctatttggcgctggcgcatatgattaagttccgcggtcattttttgatcgagggcgacctgaatccggataacagcgacgttgacaaactgtttatccagctggtgcagacctataaccagttgttcgaagagaacccgattaatgcgtctggcgtggacgcgaaggcgatcctgagcgctcgtctgtccaagtcacgccgcctggagaacttgatcgcgcaactgccgggtgagaagaaaaacggcttgttcggcaatctgattgctctgtccctaggtttgaccccgaatttcaaatctaattttgatctggcggaagatgcgaaattacagttgagcaaggacacttacgatgacgatctggacaacctgctcgctcagatcggcgatcagtatgccgatcttttcctggccgcgaagaacctgagcgatgccatcttgctatcggacatcttacgtgttaacaccgaaatcaccaaagctccgctaagcgcgtccatgattaaacgttacgatgaacaccaccaggatctgacgctgctgaaagcgttggtgcgccagcaactgccggaaaaatacaaggaaattttcttcgaccaaagcaagaacggttatgcaggctatatcgacggtggtgcgtcgcaagaagaattttataaattcatcaaaccgattttggagaagatggatggtaccgaggagctgctggtaaaattgaatcgggaagacctcttgcgaaagcagcgtactttcgataatggtagcatcccgcaccagattcatctgggcgaacttcacgcaatcctacgtcgtcaagaggatttctacccgtttctgaaggataaccgtgaaaagatcgagaagattctgacctttcgtattccgtattatgtgggtccgctggcgagaggcaacagccgttttgcgtggatgacccgcaagtcggaagagactatcacgccgtggaatttcgaggaggtcgtggacaagggtgcatcagcgcaatcttttattgaacgtatgaccaattttgataaaaaccttccaaatgaaaaagtgttgccgaagcacagcttgctgtacgagtatttcaccgtttacaatgaactgacgaaagtcaagtacgttaccgaaggtatgcgtaagccagcctttctgtctggggagcaaaaaaaagccattgtggatctgctgttcaagaccaatcgcaaggttaccgtcaagcaactgaaagaggattacttcaaaaaaatcgaatgttttgactccgtggaaatcagcggcgttgaagaccgctttaatgcatctctgggcacctaccacgatctactcaagatcatcaaggataaggacttcctggacaacgaagaaaatgaggatatcttggaagatattgtgctgactttaacgttgttcgaggatcgtgagatgattgaagaacgcctcaaaacgtacgcacatctgtttgacgataaggtgatgaaacagctaaagcgccgtcgttacaccggttggggtcgtctgtctagaaagctgattaacggcattcgtgacaaacaatctggcaagaccatcctggacttcctcaaatccgacggcttcgcaaatcgtaatttcatgcaactgatccatgatgatagcctgacctttaaagaagatattcagaaggctcaagtgtccggccagggtgactcgctgcatgagcatatcgcgaatctggctggctcccctgcgataaaaaaaggtattctccaaacggtgaaagttgtggatgaactggttaaagtcatgggtcgtcataaaccggaaaacatcgtgattgagatggcacgtgagaaccagaccacccagaaaggtcaaaaaaacagtcgtgagcgtatgaaacgtatcgaagaggggattaaggagttaggcagccagattctgaaggagcacccggttgagaacacccagctgcaaaacgagaagctctacctgtattacctgcaaaacggccgtgacatgtatgttgaccaggagctggacatcaatcgtctcagcgactacgacgtggatgctatcgtgccgcagagctttctaaaagatgattccatcgacaataaagtgctcacccgtagcgacaagaaccgtggtaaaagcgacaacgtgccgagcgaggaagtagttaagaaaatgaaaaactactggcgtcagctattgaacgccaagcttatcacccagcgtaagtttgacaacttgacgaaggcagagcgtggcggcctgagcgagctagacaaggcgggttttattaaacgccagctggtggaaactcgccagattaccaaacacgttgcgcaaatcttggactcacgcatgaatacgaagtacgacgaaaacgataaattgatccgcgaggtgaaggtcataaccctgaaatctaaactggttagcgatttccggaaggactttcaattttataaagtccgggagattaacaactatcaccatgcacacgacgcttacttaaatgcggttgttggcacggccctgatcaagaagtacccgaaactggaaagcgagtttgtttatggtgactacaaggtgtatgacgttcgcaagatgattgcaaaatccgaacaagagattggtaaggcgacggctaagtattttttctacagcaatatcatgaacttcttcaagactgaaatcaccctggcgaacggtgaaattcgtaaacgtccgttgatcgaaaccaacggtgaaaccggtgaaatcgtttgggataaaggtagggattttgcgaccgttcgtaaggtcctgagcatgccgcaggttaacatcgtgaaaaagaccgaagtgcagaccggcggtttctccaaggagagtatcttaccgaagcgcaacagtgacaagctgatcgcgcgtaaaaaagactgggatccgaaaaaatacggcggctttgactccccgaccgtcgcgtacagcgtactggtcgtggctaaagtggagaaaggcaaatcgaaaaagctgaaatccgttaaagagttgctgggtattacgattatggaacgttcgagcttcgagaaaaatccgatcgacttcctggaggccaaaggctacaaggaagttaaaaaagacctgatcattaaactgccgaagtacagcctgttcgagctcgagaacggtcgcaaacgcatgctggcgagcgcgggtgagctccaaaaaggcaacgaattggctctgccgagcaagtacgtgaacttcctgtatctggcctcccattatgaaaaactgaagggtagcccggaagataacgaacaaaagcagctgttcgtggaacaacataaacactatctggacgagattattgagcaaatcagcgagttcagcaaacgcgtgattctggcggatgctaatctggacaaggttctttcagcctataataaacaccgtgataagccgatccgtgaacaggcagaaaatattattcacctgttcaccctgacgaacctgggtgcgccagcagcgtttaaatacttcgacacaacgatcgaccggaagcgttataccagcaccaaagaggttcttgacgccaccttgattcaccaaagcatcacgggtctgtatgaaacccgtattgacttgagccagttgggcggagattcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 50 - DNA, 3453 bp, synthetic construct
  source 1..3453: mol_type = other DNA
atgggaagtcatcaccaccaccaccattcaggtgagaacctgtacttccagagcatggctccgaaaaagaaacgtaaggtgggtatccacggcgttccggcgatgagttcagaaacaggacccgttgctgtagacccgaccttgcgcagacgtattgagccgcacgaattcgaggtgtttttcgacccgagagaattgcgcaaagaaacctgtctgctgtacgagatcaactggggtggtcgtcatagcatctggcgtcacacctctcagaatacgaacaaacacgtggaagtcaatttcatcgagaagttcacgaccgagcgctacttctgtccgaacacccgttgttcgatcacgtggtttctgagctattctccgtgcggcgaatgcagccgtgcaattaccgagttcctgtcacgttacccgcacgtaactctgtttatctacattgcgcgtctctaccatcatgctgatccggaaaaccgtcaaggtttacgcgatctgattagctccggcgtgaccatccagattatgactgagcaggagagcggctattgctggcgcaactttgttaactattccccgtccaatgaggcgcattggcctcgttatccgcacctgtgggttcgtctatacgttctggaattgtactgcattatcctgggcctgccaccatgcttgaatattctgcgtcgcaagcagccgcaattgacctttttcaccatcgccctgcaaagctgccattatcagcgccttccgccgcacatcctgtgggcgaccggtctgaaatctggaagtgagacacccgggacttcagaatccgcgaccccggagtctggaatgccaaaacccgctgtagagtcagaattttcaaaagttttgaaaaagcactttccgggagaacgtttccgctcgagctatatgaaaagaggcggtaagatcctggctgcccaaggcgaagaggcagttgttgcgtatctacagggcaagtccgaggaggagcctccgaacttccagccaccggcgaagtgccacgtggtgaccaaatcccgtgacttcgcggaatggccgatcatgaaggcgtcggaagcgattcaacgttatatctatgcactgtcgaccaccgaacgcgcggcctgtaaaccgggcaaaagtagtgagagccatgcagcgtggttcgccgctaccggtgtcagcaaccacggctacagccacgttcagggtctgaacctgatttttgatcataccttgggccgttacgatggtgttctgaaaaaggtccaactgcgcaacgaaaaggcgagagcgcgtttagagagcattaacgcgtctagagcagatgaaggtttaccggagattaaagccgaggaggaagaagtggctacgaacgaaaccggccacctgttgcaaccgccgggtatcaatccgagcttttacgtgtatcagaccattagcccgcaggcataccgtccgcgtgacgagatcgtgctgccgccggagtacgcaggttacgtacgtgacccgaatgctccgattccgctgggtgtggtgcgtaaccgttgcgatatccaaaaagggtgcccgggctacatcccggaatggcagcgtgaagcaggtaccgcgatcagcccgaaaacaggcaaggccgtaactgtgccgggcctgtctccgaagaaaaacaaacgtatgcgtaggtactggcgctctgagaaggagaaagctcaagatgcgctgttggttaccgtacgcattggtaccgattgggttgtcatcgacgtgcgcggtctgttgcgcaacgcccgttggcgcaccatcgctcccaaggacatcagcctgaatgcgctcctcgatttgtttaccggtgatccggtgatcgatgtgcgtcgcaatattgttacctttacgtacaccctcgacgcctgtggtacctacgcccgtaaatggaccttgaaaggcaaacagacgaaagcgaccctggacaaactgacggccactcagacggttgcgttggttgcgattgcgcttggtcagaccaacccgatttccgctggcattagccgtgttacacaagaaaatggtgcactgcaatgcgaaccgctggaccggttcaccctgccggatgacctgttgaaggacatcagcgcatatcgcattgcttgggacagaaacgaagaggaactacgcgctcgcagcgtcgaggccctgccggaggcgcaacaagcggaggtccgtgcgctggacggggtgtccaaagaaaccgcgcgtacccaactgtgcgcagatttcggcctggacccgaaacgtctgccgtgggataaaatgagctccaatactacctttatcagcgaagccttgctgtctaacagcgtgagccgtgatcaggtttttttcactccggctccgaagaagggtgctaagaaaaaagcgcctgttgaagttatgcgtaaagaccgcacttgggcacgtgcgtacaagccgcgtctgagcgtcgaagctcagaaactgaagaacgaagccctgtgggcacttaagcgcacctccccggagtatttgaagctgagccgtcgcaaagaggagctgtgtcgtcgtagcatcaactatgttatcgaaaagacccgtcgccgtacgcaatgtcagattgtgatcccggtgatcgaggatctgaatgttcgcttctttcatggttctggtaagagactgcccggttgggataatttcttcaccgctaaaaaagagaaccgttggttcatccagggcctgcacaaagcgtttagcgatctgcgtacgcaccgttccttctatgtttttgaagttcgtccggagcgcacctctattacctgtccaaagtgcggtcattgcgaggtgggtaatcgtgacggtgaggcgtttcagtgcctgagctgcggcaagacgtgcaacgcagatcttgacgtcgcgacccataatctgacccaagtggcgttgaccggtaagaccatgccgaagcgcgaggagccgcgtgacgctcagggtacagcgccagcgcgcaagactaagaaggcgagcaaatcaaaggcgccaccggcggagcgcgaagaccagaccccggcgcaagagccaagccagaccagttctggaggtagcgggggtagcggtggtagcactaacttaagtgatattatcgagaaagaaaccgggaaacagctggtgatccaagaaagcattctgatgctgcccgaagaagttgaagaagtgattggtaacaagcctgagtcggacatcttagtgcacaccgcgtacgatgaaagcacggatgagaatgtgatgctgctcacctctgatgcccccgaatacaagccgtgggctctggtgatccaggactctaacggagaaaacaaaattaagatgctctctggcggctccatggcgcctaaaaaaaaacgcaaagtggggattcacggggttccggcgtaa

SEQ ID NO: 51 - DNA, 4869 bp, synthetic construct
  source 1..4869: mol_type = other DNA
atgggaagtcatcaccaccaccaccattcaggtgagaacctgtacttccagagcatggctccgaaaaagaaacgtaaggtgggtatccacggcgttccggcgatgagttcagaaacaggacccgttgctgtagacccgaccttgcgcagacgtattgagccgcacgaattcgaggtgtttttcgacccgagagaattgcgcaaagaaacctgtctgctgtacgagatcaactggggtggtcgtcatagcatctggcgtcacacctctcagaatacgaacaaacacgtggaagtcaatttcatcgagaagttcacgaccgagcgctacttctgtccgaacacccgttgttcgatcacgtggtttctgagctattctccgtgcggcgaatgcagccgtgcaattaccgagttcctgtcacgttacccgcacgtaactctgtttatctacattgcgcgtctctaccatcatgctgatccggaaaaccgtcaaggtttacgcgatctgattagctccggcgtgaccatccagattatgactgagcaggagagcggctattgctggcgcaactttgttaactattccccgtccaatgaggcgcattggcctcgttatccgcacctgtgggttcgtctatacgttctggaattgtactgcattatcctgggcctgccaccatgcttgaatattctgcgtcgcaagcagccgcaattgacctttttcaccatcgccctgcaaagctgccattatcagcgccttccgccgcacatcctgtgggcgaccggtctgaaatctggaagtgagacacccgggacttcagaatccgcgaccccggagtctggaatgtcaaaactagaaaagtttacaaattgttactccctgtcgaaaaccctgagattcaaagcgatcccggtgggtaaaactcaagagaacattgataacaaacgtctgctggttgaggacgaaaagcgcgcggaggattacaaaggcgttaaaaagctgttggaccgttattacctgtcctttattaatgatgttctgcacagcattaaattaaagaacctaaacaattacatctccctcttccgtaaaaagacccgcaccgaaaaggaaaataaagaactggagaaccttgagattaatctgcgtaaagaaatcgctaaagcgtttaaaggcaacgagggttataaatccttatttaaaaaagacatcatcgaaacgattctcccagaattcctggacgacaaagacgagatcgcgctggttaattccttcaacggctttaccaccgcattcaccggcttttttgataaccgcgaaaacatgttctctgaagaggccaaaagcacctcaattgcgtttcgctgcattaacgaaaatctgacgcgttatatctctaacatggatatcttcgagaaggttgacgctatctttgataaacatgaagtccaggagatcaaggagaagatcctgaacagcgactacgacgtggaggatttcttcgagggtgagttcttcaattttgttttgacccaagaaggcatcgatgtctacaatgcgataattggtggttttgttaccgagagcggggagaaaattaagggtctgaacgaatatatcaacttgtacaaccagaaaaccaaacaaaaactgccgaagtttaagccgctgtacaagcaagtcttgtcggatcgtgaatcgctgagcttttacggcgaaggctacaccagcgatgaggaggtgttggaggtgttccgcaatacgttgaataagaactctgaaattttttccagcattaagaagctggaaaaactattcaagaacttcgacgagtatagcagcgcaggtatctttgtgaaaaacggtccggctattagcaccatctcgaaagacattttcggcgaatggaatgttattcgtgataagtggaatgccgaatacgacgacattcatctgaagaaaaaggcggttgtaactgaaaagtacgaggacgatcgtcgtaagtcgttcaaaaagatcggctccttctctctggagcaactgcaagagtacgccgatgccgatttgtcagtcgtggagaagctgaaagagatcatcattcaaaaagttgacgaaatctataaggtctatggtagcagtgaaaaactgtttgacgcggacttcgtgctggagaaaagcctgaagaaaaatgacgccgtggtggcaatcatgaaagatcttctggatagcgtcaagtctttcgaaaactacatcaaagcgtttttcggtgaaggcaaggaaacgaatcgtgacgagagcttctatggtgatttcgtcctagcctatgatatcctgcttaaggtggatcacatctatgatgccatccgcaactatgttacccagaaaccgtactcaaaggacaagttcaagttatacttccagaatccgcagtttatgggtggctgggataaagataaggaaaccgactatcgcgccaccattttacgttacggtagtaagtactatctggcaatcatggataaaaaatatgcgaaatgcctgcaaaagatcgacaaggacgatgttaacggcaattatgagaagatcaactacaaactgctgccgggcccaaataagatgctgccgaaggtgtttttcagcaaaaaatggatggcgtattacaatccgagcgaggacatccaaaagatctacaaaaacggtacgtttaagaagggtgatatgttcaacctcaacgattgtcataaacttattgacttcttcaaggatagcatatctcgttatccgaagtggagcaacgcgtatgactttaactttagcgaaaccgagaaatacaaagatatcgcgggtttctatcgtgaagttgaagaacagggttacaaagtgtccttcgagtccgcgagcaagaaggaggtagacaagctggtcgaggagggtaagttgtacatgtttcagatttacaataaggacttttccgacaagtcccatggtacaccgaatttgcacaccatgtatttcaagcttctgttcgacgagaacaaccacggccagatccgtctgagcggtggtgcagagctgttcatgcgtagagctagcctgaagaaagaagagttggtcgttcatccggctaattcgccgatcgcgaacaaaaatccagataaccctaaaaagactacgaccctgagctatgatgtgtataaagacaaacgctttagcgaagaccagtatgagttacacattccgatcgctatcaacaagtgcccgaagaatattttcaagatcaacaccgaagtgcgtgttcttttgaagcacgatgacaacccgtatgtgattggtattgctcgtggtgaacgtaatctgctgtacattgtggttgtggacggcaaaggaaacatcgtggaacagtacagcctgaacgaaatcattaacaatttcaacggcattcgcattaagacggattaccattctttgctggacaaaaaggagaaagagcgtttcgaggcccgtcagaactggacctccatcgagaacattaaggagctgaaggcgggttacatcagccaggtggtgcacaaaatctgcgaactggtggagaagtacgacgcggtgattgctttggcggatttgaactccggttttaagaacagccgcgttaaagtggaaaagcaggtttatcagaaattcgaaaaaatgctgattgataaattgaattacatggttgacaaaaagtctaacccgtgtgcaaccggcggtgcgctgaagggctaccaaatcaccaataagtttgaaagctttaagagcatgagcacgcaaaatggcttcattttttacattccggcgtggctgaccagcaagatcgatccgtcaaccggctttgttaatttactgaagacgaagtatacctctattgccgactctaaaaaatttattagttccttcgatagaatcatgtatgttccggaagaggacctgttcgaatttgcactggactacaagaactttagccgtaccgatgctgattatataaagaaatggaaactctacagctatggcaaccgcatccgtatctttcgcaacccgaaaaaaaacaacgtgttcgactgggaagaggtatgtctgacttcagcgtacaaggagcttttcaataaatatggtatcaactaccagcagggtgatatccgtgcactgctttgcgagcaaagcgataaagcgttctattcaagcttcatggcgctgatgagcctgatgctgcaaatgcgcaacagcatcaccggtcgtaccgatgttgcatttctgatttctccagtaaaaaacagcgatggtatcttttacgacagccgtaattacgaggcccaagaaaatgcgattttgccgaagaacgctgacgcgaatggtgcatacaacattgcaaggaaagttctgtgggcaatcggccaatttaaaaaagcggaagatgagaagctggacaaagtgaaaattgcgattagcaacaaggagtggctggagtatgcgcagacctccgttaaatcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 52 - DNA, 5292 bp, synthetic construct
  source 1..5292: mol_type = other DNA
atgggaagtcatcaccaccaccaccattcaggtgagaacctgtacttccagagcatggctccgaaaaagaaacgtaaggtgggtatccacggcgttccggcgatgtcaagtgaaacaggacccgttgctgtagacccgaccttgagacgccgcattgagccgcacgaatttgaagttttcttcgacccgcgtgaattgcgtaaagagacgtgcttgctgtacgagattaactggggtggccgtcacagcatttggcgccacaccagccaaaacaccaacaaacatgtggaagtcaacttcatcgagaagttcactaccgagcgttacttttgtccgaatacgcgttgttcgatcacctggtttctgtcctattccccgtgcggtgaatgctcacgcgcaatcactgagttcctgtctcgttacccgcacgtaaccctgtttatctatatcgcgcgtttgtaccatcatgcggatccggagaaccgccagggtctggaagatttaattagcagcggcgttacgatccagattatgaccgagcaggagagcggctattgctggcgtaatttcgtgaactattccccgtctaatgaagcgcattggccgcgttacccacacctgtgggttcgtctctacgtgctggaattgtactgcattatcctgggtttaccgccttgcctgaatattctacgtcgcaagcaaccgcagctgacctttttcaccatcgccctgcaaagctgtcattatcagcgtctgccgccacacatcctgtgggctaccggccttaaatctggaagtgagacacccgggacttcagaatccgcgaccccggagtctggaatggataaaaagtattcaataggactagctattggcaccaacagcgttggttgggcagttatcaccgatgaatataaggtcccgagcaagaagtttaaggtcctgggcaacaccgatcgccacagcataaaaaagaaccttattggtgcgctgctgttcgattctggtgagactgccgaagcgacccgtctgaagagaaccgcgcgtcgtagatacacccggcgcaagaaccgtatctgctatctgcaagaaattttcagcaacgagatggctaaggtcgacgactccttctttcatcgcctggaggaaagctttctggttgaggaagacaaaaagcacgagcgtcatccgattttcggtaacattgttgatgaagttgcctaccacgaaaaataccccacaatttatcatctgcgtaaaaaactggtagactccaccgataaggcggatttacgccttatctatttggcgctggcgcatatgattaagttccgcggtcattttttgatcgagggcgacctgaatccggataacagcgacgttgacaaactgtttatccagctggtgcagacctataaccagttgttcgaagagaacccgattaatgcgtctggcgtggacgcgaaggcgatcctgagcgctcgtctgtccaagtcacgccgcctggagaacttgatcgcgcaactgccgggtgagaagaaaaacggcttgttcggcaatctgattgctctgtccctaggtttgaccccgaatttcaaatctaattttgatctggcggaagatgcgaaattacagttgagcaaggacacttacgatgacgatctggacaacctgctcgctcagatcggcgatcagtatgccgatcttttcctggccgcgaagaacctgagcgatgccatcttgctatcggacatcttacgtgttaacaccgaaatcaccaaagctccgctaagcgcgtccatgattaaacgttacgatgaacaccaccaggatctgacgctgctgaaagcgttggtgcgccagcaactgccggaaaaatacaaggaaattttcttcgaccaaagcaagaacggttatgcaggctatatcgacggtggtgcgtcgcaagaagaattttataaattcatcaaaccgattttggagaagatggatggtaccgaggagctgctggtaaaattgaatcgggaagacctcttgcgaaagcagcgtactttcgataatggtagcatcccgcaccagattcatctgggcgaacttcacgcaatcctacgtcgtcaagaggatttctacccgtttctgaaggataaccgtgaaaagatcgagaagattctgacctttcgtattccgtattatgtgggtccgctggcgagaggcaacagccgttttgcgtggatgacccgcaagtcggaagagactatcacgccgtggaatttcgaggaggtcgtggacaagggtgcatcagcgcaatcttttattgaacgtatgaccaattttgataaaaaccttccaaatgaaaaagtgttgccgaagcacagcttgctgtacgagtatttcaccgtttacaatgaactgacgaaagtcaagtacgttaccgaaggtatgcgtaagccagcctttctgtctggggagcaaaaaaaagccattgtggatctgctgttcaagaccaatcgcaaggttaccgtcaagcaactgaaagaggattacttcaaaaaaatcgaatgttttgactccgtggaaatcagcggcgttgaagaccgctttaatgcatctctgggcacctaccacgatctactcaagatcatcaaggataaggacttcctggacaacgaagaaaatgaggatatcttggaagatattgtgctgactttaacgttgttcgaggatcgtgagatgattgaagaacgcctcaaaacgtacgcacatctgtttgacgataaggtgatgaaacagctaaagcgccgtcgttacaccggttggggtcgtctgtctagaaagctgattaacggcattcgtgacaaacaatctggcaagaccatcctggacttcctcaaatccgacggcttcgcaaatcgtaatttcatgcaactgatccatgatgatagcctgacctttaaagaagatattcagaaggctcaagtgtccggccagggtgactcgctgcatgagcatatcgcgaatctggctggctcccctgcgataaaaaaaggtattctccaaacggtgaaagttgtggatgaactggttaaagtcatgggtcgtcataaaccggaaaacatcgtgattgagatggcacgtgagaaccagaccacccagaaaggtcaaaaaaacagtcgtgagcgtatgaaacgtatcgaagaggggattaaggagttaggcagccagattctgaaggagcacccggttgagaacacccagctgcaaaacgagaagctctacctgtattacctgcaaaacggccgtgacatgtatgttgaccaggagctggacatcaatcgtctcagcgactacgacgtggatgctatcgtgccgcagagctttctaaaagatgattccatcgacaataaagtgctcacccgtagcgacaagaaccgtggtaaaagcgacaacgtgccgagcgaggaagtagttaagaaaatgaaaaactactggcgtcagctattgaacgccaagcttatcacccagcgtaagtttgacaacttgacgaaggcagagcgtggcggcctgagcgagctagacaaggcgggttttattaaacgccagctggtggaaactcgccagattaccaaacacgttgcgcaaatcttggactcacgcatgaatacgaagtacgacgaaaacgataaattgatccgcgaggtgaaggtcataaccctgaaatctaaactggttagcgatttccggaaggactttcaattttataaagtccgggagattaacaactatcaccatgcacacgacgcttacttaaatgcggttgttggcacggccctgatcaagaagtacccgaaactggaaagcgagtttgtttatggtgactacaaggtgtatgacgttcgcaagatgattgcaaaatccgaacaagagattggtaaggcgacggctaagtattttttctacagcaatatcatgaacttcttcaagactgaaatcaccctggcgaacggtgaaattcgtaaacgtccgttgatcgaaaccaacggtgaaaccggtgaaatcgtttgggataaaggtagggattttgcgaccgttcgtaaggtcctgagcatgccgcaggttaacatcgtgaaaaagaccgaagtgcagaccggcggtttctccaaggagagtatcttaccgaagcgcaacagtgacaagctgatcgcgcgtaaaaaagactgggatccgaaaaaatacggcggctttgactccccgaccgtcgcgtacagcgtactggtcgtggctaaagtggagaaaggcaaatcgaaaaagctgaaatccgttaaagagttgctgggtattacgattatggaacgttcgagcttcgagaaaaatccgatcgacttcctggaggccaaaggctacaaggaagttaaaaaagacctgatcattaaactgccgaagtacagcctgttcgagctcgagaacggtcgcaaacgcatgctggcgagcgcgggtgagctccaaaaaggcaacgaattggctctgccgagcaagtacgtgaacttcctgtatctggcctcccattatgaaaaactgaagggtagcccggaagataacgaacaaaagcagctgttcgtggaacaacataaacactatctggacgagattattgagcaaatcagcgagttcagcaaacgcgtgattctggcggatgctaatctggacaaggttctttcagcctataataaacaccgtgataagccgatccgtgaacaggcagaaaatattattcacctgttcaccctgacgaacctgggtgcgccagcagcgtttaaatacttcgacacaacgatcgaccggaagcgttataccagcaccaaagaggttcttgacgccaccttgattcaccaaagcatcacgggtctgtatgaaacccgtattgacttgagccagttgggcggagattcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 53 - DNA, 3453 bp, synthetic construct
  source 1..3453: mol_type = other DNA
atgggaagtcatcaccaccaccaccattcaggtgagaacctgtacttccagagcatggctccgaaaaagaaacgtaaggtgggtatccacggcgttccggcgatgtcaagtgaaacaggacccgttgctgtagacccgaccttgagacgccgcattgagccgcacgaatttgaagttttcttcgacccgcgtgaattgcgtaaagagacgtgcttgctgtacgagattaactggggtggccgtcacagcatttggcgccacaccagccaaaacaccaacaaacatgtggaagtcaacttcatcgagaagttcactaccgagcgttacttttgtccgaatacgcgttgttcgatcacctggtttctgtcctattccccgtgcggtgaatgctcacgcgcaatcactgagttcctgtctcgttacccgcacgtaaccctgtttatctatatcgcgcgtttgtaccatcatgcggatccggagaaccgccagggtctggaagatttaattagcagcggcgttacgatccagattatgaccgagcaggagagcggctattgctggcgtaatttcgtgaactattccccgtctaatgaagcgcattggccgcgttacccacacctgtgggttcgtctctacgtgctggaattgtactgcattatcctgggtttaccgccttgcctgaatattctacgtcgcaagcaaccgcagctgacctttttcaccatcgccctgcaaagctgtcattatcagcgtctgccgccacacatcctgtgggctaccggccttaaatctggaagtgagacacccgggacttcagaatccgcgaccccggagtctggaatgccaaaacccgctgtagagtcagaattttcaaaagttttgaaaaagcactttccgggagaacgtttccgctcgagctatatgaaaagaggcggtaagatcctggctgcccaaggcgaagaggcagttgttgcgtatctacagggcaagtccgaggaggagcctccgaacttccagccaccggcgaagtgccacgtggtgaccaaatcccgtgacttcgcggaatggccgatcatgaaggcgtcggaagcgattcaacgttatatctatgcactgtcgaccaccgaacgcgcggcctgtaaaccgggcaaaagtagtgagagccatgcagcgtggttcgccgctaccggtgtcagcaaccacggctacagccacgttcagggtctgaacctgatttttgatcataccttgggccgttacgatggtgttctgaaaaaggtccaactgcgcaacgaaaaggcgagagcgcgtttagagagcattaacgcgtctagagcagatgaaggtttaccggagattaaagccgaggaggaagaagtggctacgaacgaaaccggccacctgttgcaaccgccgggtatcaatccgagcttttacgtgtatcagaccattagcccgcaggcataccgtccgcgtgacgagatcgtgctgccgccggagtacgcaggttacgtacgtgacccgaatgctccgattccgctgggtgtggtgcgtaaccgttgcgatatccaaaaagggtgcccgggctacatcccggaatggcagcgtgaagcaggtaccgcgatcagcccgaaaacaggcaaggccgtaactgtgccgggcctgtctccgaagaaaaacaaacgtatgcgtaggtactggcgctctgagaaggagaaagctcaagatgcgctgttggttaccgtacgcattggtaccgattgggttgtcatcgacgtgcgcggtctgttgcgcaacgcccgttggcgcaccatcgctcccaaggacatcagcctgaatgcgctcctcgatttgtttaccggtgatccggtgatcgatgtgcgtcgcaatattgttacctttacgtacaccctcgacgcctgtggtacctacgcccgtaaatggaccttgaaaggcaaacagacgaaagcgaccctggacaaactgacggccactcagacggttgcgttggttgcgattgcgcttggtcagaccaacccgatttccgctggcattagccgtgttacacaagaaaatggtgcactgcaatgcgaaccgctggaccggttcaccctgccggatgacctgttgaaggacatcagcgcatatcgcattgcttgggacagaaacgaagaggaactacgcgctcgcagcgtcgaggccctgccggaggcgcaacaagcggaggtccgtgcgctggacggggtgtccaaagaaaccgcgcgtacccaactgtgcgcagatttcggcctggacccgaaacgtctgccgtgggataaaatgagctccaatactacctttatcagcgaagccttgctgtctaacagcgtgagccgtgatcaggtttttttcactccggctccgaagaagggtgctaagaaaaaagcgcctgttgaagttatgcgtaaagaccgcacttgggcacgtgcgtacaagccgcgtctgagcgtcgaagctcagaaactgaagaacgaagccctgtgggcacttaagcgcacctccccggagtatttgaagctgagccgtcgcaaagaggagctgtgtcgtcgtagcatcaactatgttatcgaaaagacccgtcgccgtacgcaatgtcagattgtgatcccggtgatcgaggatctgaatgttcgcttctttcatggttctggtaagagactgcccggttgggataatttcttcaccgctaaaaaagagaaccgttggttcatccagggcctgcacaaagcgtttagcgatctgcgtacgcaccgttccttctatgtttttgaagttcgtccggagcgcacctctattacctgtccaaagtgcggtcattgcgaggtgggtaatcgtgacggtgaggcgtttcagtgcctgagctgcggcaagacgtgcaacgcagatcttgacgtcgcgacccataatctgacccaagtggcgttgaccggtaagaccatgccgaagcgcgaggagccgcgtgacgctcagggtacagcgccagcgcgcaagactaagaaggcgagcaaatcaaaggcgccaccggcggagcgcgaagaccagaccccggcgcaagagccaagccagaccagttctggaggtagcgggggtagcggtggtagcactaacttaagtgatattatcgagaaagaaaccgggaaacagctggtgatccaagaaagcattctgatgctgcccgaagaagttgaagaagtgattggtaacaagcctgagtcggacatcttagtgcacaccgcgtacgatgaaagcacggatgagaatgtgatgctgctcacctctgatgcccccgaatacaagccgtgggctctggtgatccaggactctaacggagaaaacaaaattaagatgctctctggcggctccatggcgcctaaaaaaaaacgcaaagtggggattcacggggttccggcgtaa

SEQ ID NO: 54 - DNA, 4869 bp, synthetic construct
  source 1..4869: mol_type = other DNA
atgggaagtcatcaccaccaccaccattcaggtgagaacctgtacttccagagcatggctccgaaaaagaaacgtaaggtgggtatccacggcgttccggcgatgtcaagtgaaacaggacccgttgctgtagacccgaccttgagacgccgcattgagccgcacgaatttgaagttttcttcgacccgcgtgaattgcgtaaagagacgtgcttgctgtacgagattaactggggtggccgtcacagcatttggcgccacaccagccaaaacaccaacaaacatgtggaagtcaacttcatcgagaagttcactaccgagcgttacttttgtccgaatacgcgttgttcgatcacctggtttctgtcctattccccgtgcggtgaatgctcacgcgcaatcactgagttcctgtctcgttacccgcacgtaaccctgtttatctatatcgcgcgtttgtaccatcatgcggatccggagaaccgccagggtctggaagatttaattagcagcggcgttacgatccagattatgaccgagcaggagagcggctattgctggcgtaatttcgtgaactattccccgtctaatgaagcgcattggccgcgttacccacacctgtgggttcgtctctacgtgctggaattgtactgcattatcctgggtttaccgccttgcctgaatattctacgtcgcaagcaaccgcagctgacctttttcaccatcgccctgcaaagctgtcattatcagcgtctgccgccacacatcctgtgggctaccggccttaaatctggaagtgagacacccgggacttcagaatccgcgaccccggagtctggaatgtcaaaactagaaaagtttacaaattgttactccctgtcgaaaaccctgagattcaaagcgatcccggtgggtaaaactcaagagaacattgataacaaacgtctgctggttgaggacgaaaagcgcgcggaggattacaaaggcgttaaaaagctgttggaccgttattacctgtcctttattaatgatgttctgcacagcattaaattaaagaacctaaacaattacatctccctcttccgtaaaaagacccgcaccgaaaaggaaaataaagaactggagaaccttgagattaatctgcgtaaagaaatcgctaaagcgtttaaaggcaacgagggttataaatccttatttaaaaaagacatcatcgaaacgattctcccagaattcctggacgacaaagacgagatcgcgctggttaattccttcaacggctttaccaccgcattcaccggcttttttgataaccgcgaaaacatgttctctgaagaggccaaaagcacctcaattgcgtttcgctgcattaacgaaaatctgacgcgttatatctctaacatggatatcttcgagaaggttgacgctatctttgataaacatgaagtccaggagatcaaggagaagatcctgaacagcgactacgacgtggaggatttcttcgagggtgagttcttcaattttgttttgacccaagaaggcatcgatgtctacaatgcgataattggtggttttgttaccgagagcggggagaaaattaagggtctgaacgaatatatcaacttgtacaaccagaaaaccaaacaaaaactgccgaagtttaagccgctgtacaagcaagtcttgtcggatcgtgaatcgctgagcttttacggcgaaggctacaccagcgatgaggaggtgttggaggtgttccgcaatacgttgaataagaactctgaaattttttccagcattaagaagctggaaaaactattcaagaacttcgacgagtatagcagcgcaggtatctttgtgaaaaacggtccggctattagcaccatctcgaaagacattttcggcgaatggaatgttattcgtgataagtggaatgccgaatacgacgacattcatctgaagaaaaaggcggttgtaactgaaaagtacgaggacgatcgtcgtaagtcgttcaaaaagatcggctccttctctctggagcaactgcaagagtacgccgatgccgatttgtcagtcgtggagaagctgaaagagatcatcattcaaaaagttgacgaaatctataaggtctatggtagcagtgaaaaactgtttgacgcggacttcgtgctggagaaaagcctgaagaaaaatgacgccgtggtggcaatcatgaaagatcttctggatagcgtcaagtctttcgaaaactacatcaaagcgtttttcggtgaaggcaaggaaacgaatcgtgacgagagcttctatggtgatttcgtcctagcctatgatatcctgcttaaggtggatcacatctatgatgccatccgcaactatgttacccagaaaccgtactcaaaggacaagttcaagttatacttccagaatccgcagtttatgggtggctgggataaagataaggaaaccgactatcgcgccaccattttacgttacggtagtaagtactatctggcaatcatggataaaaaatatgcgaaatgcctgcaaaagatcgacaaggacgatgttaacggcaattatgagaagatcaactacaaactgctgccgggcccaaataagatgctgccgaaggtgtttttcagcaaaaaatggatggcgtattacaatccgagcgaggacatccaaaagatctacaaaaacggtacgtttaagaagggtgatatgttcaacctcaacgattgtcataaacttattgacttcttcaaggatagcatatctcgttatccgaagtggagcaacgcgtatgactttaactttagcgaaaccgagaaatacaaagatatcgcgggtttctatcgtgaagttgaagaacagggttacaaagtgtccttcgagtccgcgagcaagaaggaggtagacaagctggtcgaggagggtaagttgtacatgtttcagatttacaataaggacttttccgacaagtcccatggtacaccgaatttgcacaccatgtatttcaagcttctgttcgacgagaacaaccacggccagatccgtctgagcggtggtgcagagctgttcatgcgtagagctagcctgaagaaagaagagttggtcgttcatccggctaattcgccgatcgcgaacaaaaatccagataaccctaaaaagactacgaccctgagctatgatgtgtataaagacaaacgctttagcgaagaccagtatgagttacacattccgatcgctatcaacaagtgcccgaagaatattttcaagatcaacaccgaagtgcgtgttcttttgaagcacgatgacaacccgtatgtgattggtattgctcgtggtgaacgtaatctgctgtacattgtggttgtggacggcaaaggaaacatcgtggaacagtacagcctgaacgaaatcattaacaatttcaacggcattcgcattaagacggattaccattctttgctggacaaaaaggagaaagagcgtttcgaggcccgtcagaactggacctccatcgagaacattaaggagctgaaggcgggttacatcagccaggtggtgcacaaaatctgcgaactggtggagaagtacgacgcggtgattgctttggcggatttgaactccggttttaagaacagccgcgttaaagtggaaaagcaggtttatcagaaattcgaaaaaatgctgattgataaattgaattacatggttgacaaaaagtctaacccgtgtgcaaccggcggtgcgctgaagggctaccaaatcaccaataagtttgaaagctttaagagcatgagcacgcaaaatggcttcattttttacattccggcgtggctgaccagcaagatcgatccgtcaaccggctttgttaatttactgaagacgaagtatacctctattgccgactctaaaaaatttattagttccttcgatagaatcatgtatgttccggaagaggacctgttcgaatttgcactggactacaagaactttagccgtaccgatgctgattatataaagaaatggaaactctacagctatggcaaccgcatccgtatctttcgcaacccgaaaaaaaacaacgtgttcgactgggaagaggtatgtctgacttcagcgtacaaggagcttttcaataaatatggtatcaactaccagcagggtgatatccgtgcactgctttgcgagcaaagcgataaagcgttctattcaagcttcatggcgctgatgagcctgatgctgcaaatgcgcaacagcatcaccggtcgtaccgatgttgcatttctgatttctccagtaaaaaacagcgatggtatcttttacgacagccgtaattacgaggcccaagaaaatgcgattttgccgaagaacgctgacgcgaatggtgcatacaacattgcaaggaaagttctgtgggcaatcggccaatttaaaaaagcggaagatgagaagctggacaaagtgaaaattgcgattagcaacaaggagtggctggagtatgcgcagacctccgttaaatcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 55 - DNA, 5187 bp, synthetic construct
  source 1..5187: mol_type = other DNA
atgggaagtcatcaccaccaccaccattcaggtgagaacctgtacttccagagcatggctccgaaaaagaaacgtaaggtgggtatccacggcgttccggcgatgaaacctcagattcgcgatcatcgtccgaatcctatggaggcgatgtatccgcatattttctactttcattttgaaaacttagaaaaagcctatggccgtaacgaaacttggctgtgctttaccgtagagattatcaaacaatatttaccggtaccctggaaaaaaggtgttttccgtaatcaggttgatccggagacacattgtcatgcagagaagtgcttcctgtcctggttctgcaataacacccttagcccgaaaaagaattaccaagttacttggtacacgagctggtcgccatgtccagagtgcgcaggagaagtagcagaattcttagctgaacattcgaacgtaaaattgacaatctacaccgcccgtctctactacctgtgggacaccgactatcaggaaggcctgcgctcattgtctgaggagggcgcatcggtcgaaatcatggattatgaagattttcagtattgttgggaaaatttcgtatacgacgatggtgagccttttaaacgttggaaaggcctgaagtacaactttcagtctttgacccgtcgcttgcgcgaaattctgcaatctggaagtgagacacccgggacttcagaatccgcgaccccggagtctggaatggataaaaagtattcaataggactagctattggcaccaacagcgttggttgggcagttatcaccgatgaatataaggtcccgagcaagaagtttaaggtcctgggcaacaccgatcgccacagcataaaaaagaaccttattggtgcgctgctgttcgattctggtgagactgccgaagcgacccgtctgaagagaaccgcgcgtcgtagatacacccggcgcaagaaccgtatctgctatctgcaagaaattttcagcaacgagatggctaaggtcgacgactccttctttcatcgcctggaggaaagctttctggttgaggaagacaaaaagcacgagcgtcatccgattttcggtaacattgttgatgaagttgcctaccacgaaaaataccccacaatttatcatctgcgtaaaaaactggtagactccaccgataaggcggatttacgccttatctatttggcgctggcgcatatgattaagttccgcggtcattttttgatcgagggcgacctgaatccggataacagcgacgttgacaaactgtttatccagctggtgcagacctataaccagttgttcgaagagaacccgattaatgcgtctggcgtggacgcgaaggcgatcctgagcgctcgtctgtccaagtcacgccgcctggagaacttgatcgcgcaactgccgggtgagaagaaaaacggcttgttcggcaatctgattgctctgtccctaggtttgaccccgaatttcaaatctaattttgatctggcggaagatgcgaaattacagttgagcaaggacacttacgatgacgatctggacaacctgctcgctcagatcggcgatcagtatgccgatcttttcctggccgcgaagaacctgagcgatgccatcttgctatcggacatcttacgtgttaacaccgaaatcaccaaagctccgctaagcgcgtccatgattaaacgttacgatgaacaccaccaggatctgacgctgctgaaagcgttggtgcgccagcaactgccggaaaaatacaaggaaattttcttcgaccaaagcaagaacggttatgcaggctatatcgacggtggtgcgtcgcaagaagaattttataaattcatcaaaccgattttggagaagatggatggtaccgaggagctgctggtaaaattgaatcgggaagacctcttgcgaaagcagcgtactttcgataatggtagcatcccgcaccagattcatctgggcgaacttcacgcaatcctacgtcgtcaagaggatttctacccgtttctgaaggataaccgtgaaaagatcgagaagattctgacctttcgtattccgtattatgtgggtccgctggcgagaggcaacagccgttttgcgtggatgacccgcaagtcggaagagactatcacgccgtggaatttcgaggaggtcgtggacaagggtgcatcagcgcaatcttttattgaacgtatgaccaattttgataaaaaccttccaaatgaaaaagtgttgccgaagcacagcttgctgtacgagtatttcaccgtttacaatgaactgacgaaagtcaagtacgttaccgaaggtatgcgtaagccagcctttctgtctggggagcaaaaaaaagccattgtggatctgctgttcaagaccaatcgcaaggttaccgtcaagcaactgaaagaggattacttcaaaaaaatcgaatgttttgactccgtggaaatcagcggcgttgaagaccgctttaatgcatctctgggcacctaccacgatctactcaagatcatcaaggataaggacttcctggacaacgaagaaaatgaggatatcttggaagatattgtgctgactttaacgttgttcgaggatcgtgagatgattgaagaacgcctcaaaacgtacgcacatctgtttgacgataaggtgatgaaacagctaaagcgccgtcgttacaccggttggggtcgtctgtctagaaagctgattaacggcattcgtgacaaacaatctggcaagaccatcctggacttcctcaaatccgacggcttcgcaaatcgtaatttcatgcaactgatccatgatgatagcctgacctttaaagaagatattcagaaggctcaagtgtccggccagggtgactcgctgcatgagcatatcgcgaatctggctggctcccctgcgataaaaaaaggtattctccaaacggtgaaagttgtggatgaactggttaaagtcatgggtcgtcataaaccggaaaacatcgtgattgagatggcacgtgagaaccagaccacccagaaaggtcaaaaaaacagtcgtgagcgtatgaaacgtatcgaagaggggattaaggagttaggcagccagattctgaaggagcacccggttgagaacacccagctgcaaaacgagaagctctacctgtattacctgcaaaacggccgtgacatgtatgttgaccaggagctggacatcaatcgtctcagcgactacgacgtggatgctatcgtgccgcagagctttctaaaagatgattccatcgacaataaagtgctcacccgtagcgacaagaaccgtggtaaaagcgacaacgtgccgagcgaggaagtagttaagaaaatgaaaaactactggcgtcagctattgaacgccaagcttatcacccagcgtaagtttgacaacttgacgaaggcagagcgtggcggcctgagcgagctagacaaggcgggttttattaaacgccagctggtggaaactcgccagattaccaaacacgttgcgcaaatcttggactcacgcatgaatacgaagtacgacgaaaacgataaattgatccgcgaggtgaaggtcataaccctgaaatctaaactggttagcgatttccggaaggactttcaattttataaagtccgggagattaacaactatcaccatgcacacgacgcttacttaaatgcggttgttggcacggccctgatcaagaagtacccgaaactggaaagcgagtttgtttatggtgactacaaggtgtatgacgttcgcaagatgattgcaaaatccgaacaagagattggtaaggcgacggctaagtattttttctacagcaatatcatgaacttcttcaagactgaaatcaccctggcgaacggtgaaattcgtaaacgtccgttgatcgaaaccaacggtgaaaccggtgaaatcgtttgggataaaggtagggattttgcgaccgttcgtaaggtcctgagcatgccgcaggttaacatcgtgaaaaagaccgaagtgcagaccggcggtttctccaaggagagtatcttaccgaagcgcaacagtgacaagctgatcgcgcgtaaaaaagactgggatccgaaaaaatacggcggctttgactccccgaccgtcgcgtacagcgtactggtcgtggctaaagtggagaaaggcaaatcgaaaaagctgaaatccgttaaagagttgctgggtattacgattatggaacgttcgagcttcgagaaaaatccgatcgacttcctggaggccaaaggctacaaggaagttaaaaaagacctgatcattaaactgccgaagtacagcctgttcgagctcgagaacggtcgcaaacgcatgctggcgagcgcgggtgagctccaaaaaggcaacgaattggctctgccgagcaagtacgtgaacttcctgtatctggcctcccattatgaaaaactgaagggtagcccggaagataacgaacaaaagcagctgttcgtggaacaacataaacactatctggacgagattattgagcaaatcagcgagttcagcaaacgcgtgattctggcggatgctaatctggacaaggttctttcagcctataataaacaccgtgataagccgatccgtgaacaggcagaaaatattattcacctgttcaccctgacgaacctgggtgcgccagcagcgtttaaatacttcgacacaacgatcgaccggaagcgttataccagcaccaaagaggttcttgacgccaccttgattcaccaaagcatcacgggtctgtatgaaacccgtattgacttgagccagttgggcggagattcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 56 - DNA, 3348 bp, synthetic construct
  source 1..3348: mol_type = other DNA
atgggaagtcatcaccaccaccaccattcaggtgagaacctgtacttccagagcatggctccgaaaaagaaacgtaaggtgggtatccacggcgttccggcgatgaaacctcagattcgcgatcatcgtccgaatcctatggaggcgatgtatccgcatattttctactttcattttgaaaacttagaaaaagcctatggccgtaacgaaacttggctgtgctttaccgtagagattatcaaacaatatttaccggtaccctggaaaaaaggtgttttccgtaatcaggttgatccggagacacattgtcatgcagagaagtgcttcctgtcctggttctgcaataacacccttagcccgaaaaagaattaccaagttacttggtacacgagctggtcgccatgtccagagtgcgcaggagaagtagcagaattcttagctgaacattcgaacgtaaaattgacaatctacaccgcccgtctctactacctgtgggacaccgactatcaggaaggcctgcgctcattgtctgaggagggcgcatcggtcgaaatcatggattatgaagattttcagtattgttgggaaaatttcgtatacgacgatggtgagccttttaaacgttggaaaggcctgaagtacaactttcagtctttgacccgtcgcttgcgcgaaattctgcaatctggaagtgagacacccgggacttcagaatccgcgaccccggagtctggaatgccaaaacccgctgtagagtcagaattttcaaaagttttgaaaaagcactttccgggagaacgtttccgctcgagctatatgaaaagaggcggtaagatcctggctgcccaaggcgaagaggcagttgttgcgtatctacagggcaagtccgaggaggagcctccgaacttccagccaccggcgaagtgccacgtggtgaccaaatcccgtgacttcgcggaatggccgatcatgaaggcgtcggaagcgattcaacgttatatctatgcactgtcgaccaccgaacgcgcggcctgtaaaccgggcaaaagtagtgagagccatgcagcgtggttcgccgctaccggtgtcagcaaccacggctacagccacgttcagggtctgaacctgatttttgatcataccttgggccgttacgatggtgttctgaaaaaggtccaactgcgcaacgaaaaggcgagagcgcgtttagagagcattaacgcgtctagagcagatgaaggtttaccggagattaaagccgaggaggaagaagtggctacgaacgaaaccggccacctgttgcaaccgccgggtatcaatccgagcttttacgtgtatcagaccattagcccgcaggcataccgtccgcgtgacgagatcgtgctgccgccggagtacgcaggttacgtacgtgacccgaatgctccgattccgctgggtgtggtgcgtaaccgttgcgatatccaaaaagggtgcccgggctacatcccggaatggcagcgtgaagcaggtaccgcgatcagcccgaaaacaggcaaggccgtaactgtgccgggcctgtctccgaagaaaaacaaacgtatgcgtaggtactggcgctctgagaaggagaaagctcaagatgcgctgttggttaccgtacgcattggtaccgattgggttgtcatcgacgtgcgcggtctgttgcgcaacgcccgttggcgcaccatcgctcccaaggacatcagcctgaatgcgctcctcgatttgtttaccggtgatccggtgatcgatgtgcgtcgcaatattgttacctttacgtacaccctcgacgcctgtggtacctacgcccgtaaatggaccttgaaaggcaaacagacgaaagcgaccctggacaaactgacggccactcagacggttgcgttggttgcgattgcgcttggtcagaccaacccgatttccgctggcattagccgtgttacacaagaaaatggtgcactgcaatgcgaaccgctggaccggttcaccctgccggatgacctgttgaaggacatcagcgcatatcgcattgcttgggacagaaacgaagaggaactacgcgctcgcagcgtcgaggccctgccggaggcgcaacaagcggaggtccgtgcgctggacggggtgtccaaagaaaccgcgcgtacccaactgtgcgcagatttcggcctggacccgaaacgtctgccgtgggataaaatgagctccaatactacctttatcagcgaagccttgctgtctaacagcgtgagccgtgatcaggtttttttcactccggctccgaagaagggtgctaagaaaaaagcgcctgttgaagttatgcgtaaagaccgcacttgggcacgtgcgtacaagccgcgtctgagcgtcgaagctcagaaactgaagaacgaagccctgtgggcacttaagcgcacctccccggagtatttgaagctgagccgtcgcaaagaggagctgtgtcgtcgtagcatcaactatgttatcgaaaagacccgtcgccgtacgcaatgtcagattgtgatcccggtgatcgaggatctgaatgttcgcttctttcatggttctggtaagagactgcccggttgggataatttcttcaccgctaaaaaagagaaccgttggttcatccagggcctgcacaaagcgtttagcgatctgcgtacgcaccgttccttctatgtttttgaagttcgtccggagcgcacctctattacctgtccaaagtgcggtcattgcgaggtgggtaatcgtgacggtgaggcgtttcagtgcctgagctgcggcaagacgtgcaacgcagatcttgacgtcgcgacccataatctgacccaagtggcgttgaccggtaagaccatgccgaagcgcgaggagccgcgtgacgctcagggtacagcgccagcgcgcaagactaagaaggcgagcaaatcaaaggcgccaccggcggagcgcgaagaccagaccccggcgcaagagccaagccagaccagttctggaggtagcgggggtagcggtggtagcactaacttaagtgatattatcgagaaagaaaccgggaaacagctggtgatccaagaaagcattctgatgctgcccgaagaagttgaagaagtgattggtaacaagcctgagtcggacatcttagtgcacaccgcgtacgatgaaagcacggatgagaatgtgatgctgctcacctctgatgcccccgaatacaagccgtgggctctggtgatccaggactctaacggagaaaacaaaattaagatgctctctggcggctccatggcgcctaaaaaaaaacgcaaagtggggattcacggggttccggcgtaa

SEQ ID NO: 57 - DNA, 4764 bp, synthetic construct
  source 1..4764: mol_type = other DNA
atgggaagtcatcaccaccaccaccattcaggtgagaacctgtacttccagagcatggctccgaaaaagaaacgtaaggtgggtatccacggcgttccggcgatgaaacctcagattcgcgatcatcgtccgaatcctatggaggcgatgtatccgcatattttctactttcattttgaaaacttagaaaaagcctatggccgtaacgaaacttggctgtgctttaccgtagagattatcaaacaatatttaccggtaccctggaaaaaaggtgttttccgtaatcaggttgatccggagacacattgtcatgcagagaagtgcttcctgtcctggttctgcaataacacccttagcccgaaaaagaattaccaagttacttggtacacgagctggtcgccatgtccagagtgcgcaggagaagtagcagaattcttagctgaacattcgaacgtaaaattgacaatctacaccgcccgtctctactacctgtgggacaccgactatcaggaaggcctgcgctcattgtctgaggagggcgcatcggtcgaaatcatggattatgaagattttcagtattgttgggaaaatttcgtatacgacgatggtgagccttttaaacgttggaaaggcctgaagtacaactttcagtctttgacccgtcgcttgcgcgaaattctgcaatctggaagtgagacacccgggacttcagaatccgcgaccccggagtctggaatgtcaaaactagaaaagtttacaaattgttactccctgtcgaaaaccctgagattcaaagcgatcccggtgggtaaaactcaagagaacattgataacaaacgtctgctggttgaggacgaaaagcgcgcggaggattacaaaggcgttaaaaagctgttggaccgttattacctgtcctttattaatgatgttctgcacagcattaaattaaagaacctaaacaattacatctccctcttccgtaaaaagacccgcaccgaaaaggaaaataaagaactggagaaccttgagattaatctgcgtaaagaaatcgctaaagcgtttaaaggcaacgagggttataaatccttatttaaaaaagacatcatcgaaacgattctcccagaattcctggacgacaaagacgagatcgcgctggttaattccttcaacggctttaccaccgcattcaccggcttttttgataaccgcgaaaacatgttctctgaagaggccaaaagcacctcaattgcgtttcgctgcattaacgaaaatctgacgcgttatatctctaacatggatatcttcgagaaggttgacgctatctttgataaacatgaagtccaggagatcaaggagaagatcctgaacagcgactacgacgtggaggatttcttcgagggtgagttcttcaattttgttttgacccaagaaggcatcgatgtctacaatgcgataattggtggttttgttaccgagagcggggagaaaattaagggtctgaacgaatatatcaacttgtacaaccagaaaaccaaacaaaaactgccgaagtttaagccgctgtacaagcaagtcttgtcggatcgtgaatcgctgagcttttacggcgaaggctacaccagcgatgaggaggtgttggaggtgttccgcaatacgttgaataagaactctgaaattttttccagcattaagaagctggaaaaactattcaagaacttcgacgagtatagcagcgcaggtatctttgtgaaaaacggtccggctattagcaccatctcgaaagacattttcggcgaatggaatgttattcgtgataagtggaatgccgaatacgacgacattcatctgaagaaaaaggcggttgtaactgaaaagtacgaggacgatcgtcgtaagtcgttcaaaaagatcggctccttctctctggagcaactgcaagagtacgccgatgccgatttgtcagtcgtggagaagctgaaagagatcatcattcaaaaagttgacgaaatctataaggtctatggtagcagtgaaaaactgtttgacgcggacttcgtgctggagaaaagcctgaagaaaaatgacgccgtggtggcaatcatgaaagatcttctggatagcgtcaagtctttcgaaaactacatcaaagcgtttttcggtgaaggcaaggaaacgaatcgtgacgagagcttctatggtgatttcgtcctagcctatgatatcctgcttaaggtggatcacatctatgatgccatccgcaactatgttacccagaaaccgtactcaaaggacaagttcaagttatacttccagaatccgcagtttatgggtggctgggataaagataaggaaaccgactatcgcgccaccattttacgttacggtagtaagtactatctggcaatcatggataaaaaatatgcgaaatgcctgcaaaagatcgacaaggacgatgttaacggcaattatgagaagatcaactacaaactgctgccgggcccaaataagatgctgccgaaggtgtttttcagcaaaaaatggatggcgtattacaatccgagcgaggacatccaaaagatctacaaaaacggtacgtttaagaagggtgatatgttcaacctcaacgattgtcataaacttattgacttcttcaaggatagcatatctcgttatccgaagtggagcaacgcgtatgactttaactttagcgaaaccgagaaatacaaagatatcgcgggtttctatcgtgaagttgaagaacagggttacaaagtgtccttcgagtccgcgagcaagaaggaggtagacaagctggtcgaggagggtaagttgtacatgtttcagatttacaataaggacttttccgacaagtcccatggtacaccgaatttgcacaccatgtatttcaagcttctgttcgacgagaacaaccacggccagatccgtctgagcggtggtgcagagctgttcatgcgtagagctagcctgaagaaagaagagttggtcgttcatccggctaattcgccgatcgcgaacaaaaatccagataaccctaaaaagactacgaccctgagctatgatgtgtataaagacaaacgctttagcgaagaccagtatgagttacacattccgatcgctatcaacaagtgcccgaagaatattttcaagatcaacaccgaagtgcgtgttcttttgaagcacgatgacaacccgtatgtgattggtattgctcgtggtgaacgtaatctgctgtacattgtggttgtggacggcaaaggaaacatcgtggaacagtacagcctgaacgaaatcattaacaatttcaacggcattcgcattaagacggattaccattctttgctggacaaaaaggagaaagagcgtttcgaggcccgtcagaactggacctccatcgagaacattaaggagctgaaggcgggttacatcagccaggtggtgcacaaaatctgcgaactggtggagaagtacgacgcggtgattgctttggcggatttgaactccggttttaagaacagccgcgttaaagtggaaaagcaggtttatcagaaattcgaaaaaatgctgattgataaattgaattacatggttgacaaaaagtctaacccgtgtgcaaccggcggtgcgctgaagggctaccaaatcaccaataagtttgaaagctttaagagcatgagcacgcaaaatggcttcattttttacattccggcgtggctgaccagcaagatcgatccgtcaaccggctttgttaatttactgaagacgaagtatacctctattgccgactctaaaaaatttattagttccttcgatagaatcatgtatgttccggaagaggacctgttcgaatttgcactggactacaagaactttagccgtaccgatgctgattatataaagaaatggaaactctacagctatggcaaccgcatccgtatctttcgcaacccgaaaaaaaacaacgtgttcgactgggaagaggtatgtctgacttcagcgtacaaggagcttttcaataaatatggtatcaactaccagcagggtgatatccgtgcactgctttgcgagcaaagcgataaagcgttctattcaagcttcatggcgctgatgagcctgatgctgcaaatgcgcaacagcatcaccggtcgtaccgatgttgcatttctgatttctccagtaaaaaacagcgatggtatcttttacgacagccgtaattacgaggcccaagaaaatgcgattttgccgaagaacgctgacgcgaatggtgcatacaacattgcaaggaaagttctgtgggcaatcggccaatttaaaaaagcggaagatgagaagctggacaaagtgaaaattgcgattagcaacaaggagtggctggagtatgcgcagacctccgttaaatcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 58 - DNA, 5445 bp, synthetic construct
  source 1..5445: mol_type = other DNA
atgggaagtcatcaccaccaccaccattcaggtgagaacctgtacttccagagcatggctccgaaaaagaaacgtaaggtgggtatccacggcgttccggcgatggatccacagaggctaagacaatggcccggtccgggtccggcgagccgcggtggctacggccagcgtccgcgtatccgcaatccggaagagtggttccacgaactgagtccgagaacctttagcttccactttcgtaacctgcgttttgctagcggtcagaatcgcagctatatctgctgccaggtggaaggcaagaactgcttcttccaaggtatttttcaaaaccaggttccgccggacccaccttgtcatgctgaactttgctttttgtcctggttccaaagctggggtttgtcgccggacgagcactattacgtgacctggttcatctcctggtccccgtgctgtgaatgtgcggcgaaagtcgcccaatttctggaagagaaccgcaacgtgagcttgtctttgtctgcggcgcgtctgtactacttctggaaaagcgagtcccgtgagggcctgcgccgtctgtccgatctgggtgcacaggttggtattatgtcgttccaagattttcagcattgttggaataactttgttcataatctgggcatgccgttccagccgtggaagaaattgcacaaaaactaccaacgtctggtgaccgaattaaaacagattctccgcgaggagccggctacctatggctctccacaggcgcaaggtaaggtgcgcatcggcagcaccgcggcgggtctgcgtcacagccatagccacacccgtagcgaggcgcatctgcgtcccaaccactcatctcgtcagcaccgtatcctgaatccgccgcgcgaggcccgtgcgcgtacgtgcgttctggtagacgcatcctggatttgctatcgttctggaagtgagacacccgggacttcagaatccgcgaccccggagtctggaatggataaaaagtattcaataggactagctattggcaccaacagcgttggttgggcagttatcaccgatgaatataaggtcccgagcaagaagtttaaggtcctgggcaacaccgatcgccacagcataaaaaagaaccttattggtgcgctgctgttcgattctggtgagactgccgaagcgacccgtctgaagagaaccgcgcgtcgtagatacacccggcgcaagaaccgtatctgctatctgcaagaaattttcagcaacgagatggctaaggtcgacgactccttctttcatcgcctggaggaaagctttctggttgaggaagacaaaaagcacgagcgtcatccgattttcggtaacattgttgatgaagttgcctaccacgaaaaataccccacaatttatcatctgcgtaaaaaactggtagactccaccgataaggcggatttacgccttatctatttggcgctggcgcatatgattaagttccgcggtcattttttgatcgagggcgacctgaatccggataacagcgacgttgacaaactgtttatccagctggtgcagacctataaccagttgttcgaagagaacccgattaatgcgtctggcgtggacgcgaaggcgatcctgagcgctcgtctgtccaagtcacgccgcctggagaacttgatcgcgcaactgccgggtgagaagaaaaacggcttgttcggcaatctgattgctctgtccctaggtttgaccccgaatttcaaatctaattttgatctggcggaagatgcgaaattacagttgagcaaggacacttacgatgacgatctggacaacctgctcgctcagatcggcgatcagtatgccgatcttttcctggccgcgaagaacctgagcgatgccatcttgctatcggacatcttacgtgttaacaccgaaatcaccaaagctccgctaagcgcgtccatgattaaacgttacgatgaacaccaccaggatctgacgctgctgaaagcgttggtgcgccagcaactgccggaaaaatacaaggaaattttcttcgaccaaagcaagaacggttatgcaggctatatcgacggtggtgcgtcgcaagaagaattttataaattcatcaaaccgattttggagaagatggatggtaccgaggagctgctggtaaaattgaatcgggaagacctcttgcgaaagcagcgtactttcgataatggtagcatcccgcaccagattcatctgggcgaacttcacgcaatcctacgtcgtcaagaggatttctacccgtttctgaaggataaccgtgaaaagatcgagaagattctgacctttcgtattccgtattatgtgggtccgctggcgagaggcaacagccgttttgcgtggatgacccgcaagtcggaagagactatcacgccgtggaatttcgaggaggtcgtggacaagggtgcatcagcgcaatcttttattgaacgtatgaccaattttgataaaaaccttccaaatgaaaaagtgttgccgaagcacagcttgctgtacgagtatttcaccgtttacaatgaactgacgaaagtcaagtacgttaccgaaggtatgcgtaagccagcctttctgtctggggagcaaaaaaaagccattgtggatctgctgttcaagaccaatcgcaaggttaccgtcaagcaactgaaagaggattacttcaaaaaaatcgaatgttttgactccgtggaaatcagcggcgttgaagaccgctttaatgcatctctgggcacctaccacgatctactcaagatcatcaaggataaggacttcctggacaacgaagaaaatgaggatatcttggaagatattgtgctgactttaacgttgttcgaggatcgtgagatgattgaagaacgcctcaaaacgtacgcacatctgtttgacgataaggtgatgaaacagctaaagcgccgtcgttacaccggttggggtcgtctgtctagaaagctgattaacggcattcgtgacaaacaatctggcaagaccatcctggacttcctcaaatccgacggcttcgcaaatcgtaatttcatgcaactgatccatgatgatagcctgacctttaaagaagatattcagaaggctcaagtgtccggccagggtgactcgctgcatgagcatatcgcgaatctggctggctcccctgcgataaaaaaaggtattctccaaacggtgaaagttgtggatgaactggttaaagtcatgggtcgtcataaaccggaaaacatcgtgattgagatggcacgtgagaaccagaccacccagaaaggtcaaaaaaacagtcgtgagcgtatgaaacgtatcgaagaggggattaaggagttaggcagccagattctgaaggagcacccggttgagaacacccagctgcaaaacgagaagctctacctgtattacctgcaaaacggccgtgacatgtatgttgaccaggagctggacatcaatcgtctcagcgactacgacgtggatgctatcgtgccgcagagctttctaaaagatgattccatcgacaataaagtgctcacccgtagcgacaagaaccgtggtaaaagcgacaacgtgccgagcgaggaagtagttaagaaaatgaaaaactactggcgtcagctattgaacgccaagcttatcacccagcgtaagtttgacaacttgacgaaggcagagcgtggcggcctgagcgagctagacaaggcgggttttattaaacgccagctggtggaaactcgccagattaccaaacacgttgcgcaaatcttggactcacgcatgaatacgaagtacgacgaaaacgataaattgatccgcgaggtgaaggtcataaccctgaaatctaaactggttagcgatttccggaaggactttcaattttataaagtccgggagattaacaactatcaccatgcacacgacgcttacttaaatgcggttgttggcacggccctgatcaagaagtacccgaaactggaaagcgagtttgtttatggtgactacaaggtgtatgacgttcgcaagatgattgcaaaatccgaacaagagattggtaaggcgacggctaagtattttttctacagcaatatcatgaacttcttcaagactgaaatcaccctggcgaacggtgaaattcgtaaacgtccgttgatcgaaaccaacggtgaaaccggtgaaatcgtttgggataaaggtagggattttgcgaccgttcgtaaggtcctgagcatgccgcaggttaacatcgtgaaaaagaccgaagtgcagaccggcggtttctccaaggagagtatcttaccgaagcgcaacagtgacaagctgatcgcgcgtaaaaaagactgggatccgaaaaaatacggcggctttgactccccgaccgtcgcgtacagcgtactggtcgtggctaaagtggagaaaggcaaatcgaaaaagctgaaatccgttaaagagttgctgggtattacgattatggaacgttcgagcttcgagaaaaatccgatcgacttcctggaggccaaaggctacaaggaagttaaaaaagacctgatcattaaactgccgaagtacagcctgttcgagctcgagaacggtcgcaaacgcatgctggcgagcgcgggtgagctccaaaaaggcaacgaattggctctgccgagcaagtacgtgaacttcctgtatctggcctcccattatgaaaaactgaagggtagcccggaagataacgaacaaaagcagctgttcgtggaacaacataaacactatctggacgagattattgagcaaatcagcgagttcagcaaacgcgtgattctggcggatgctaatctggacaaggttctttcagcctataataaacaccgtgataagccgatccgtgaacaggcagaaaatattattcacctgttcaccctgacgaacctgggtgcgccagcagcgtttaaatacttcgacacaacgatcgaccggaagcgttataccagcaccaaagaggttcttgacgccaccttgattcaccaaagcatcacgggtctgtatgaaacccgtattgacttgagccagttgggcggagattcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 59 - DNA, 3606 bp, synthetic construct
  source 1..3606: mol_type = other DNA
atgggaagtcatcaccaccaccaccattcaggtgagaacctgtacttccagagcatggctccgaaaaagaaacgtaaggtgggtatccacggcgttccggcgatggatccacagaggctaagacaatggcccggtccgggtccggcgagccgcggtggctacggccagcgtccgcgtatccgcaatccggaagagtggttccacgaactgagtccgagaacctttagcttccactttcgtaacctgcgttttgctagcggtcagaatcgcagctatatctgctgccaggtggaaggcaagaactgcttcttccaaggtatttttcaaaaccaggttccgccggacccaccttgtcatgctgaactttgctttttgtcctggttccaaagctggggtttgtcgccggacgagcactattacgtgacctggttcatctcctggtccccgtgctgtgaatgtgcggcgaaagtcgcccaatttctggaagagaaccgcaacgtgagcttgtctttgtctgcggcgcgtctgtactacttctggaaaagcgagtcccgtgagggcctgcgccgtctgtccgatctgggtgcacaggttggtattatgtcgttccaagattttcagcattgttggaataactttgttcataatctgggcatgccgttccagccgtggaagaaattgcacaaaaactaccaacgtctggtgaccgaattaaaacagattctccgcgaggagccggctacctatggctctccacaggcgcaaggtaaggtgcgcatcggcagcaccgcggcgggtctgcgtcacagccatagccacacccgtagcgaggcgcatctgcgtcccaaccactcatctcgtcagcaccgtatcctgaatccgccgcgcgaggcccgtgcgcgtacgtgcgttctggtagacgcatcctggatttgctatcgttctggaagtgagacacccgggacttcagaatccgcgaccccggagtctggaatgccaaaacccgctgtagagtcagaattttcaaaagttttgaaaaagcactttccgggagaacgtttccgctcgagctatatgaaaagaggcggtaagatcctggctgcccaaggcgaagaggcagttgttgcgtatctacagggcaagtccgaggaggagcctccgaacttccagccaccggcgaagtgccacgtggtgaccaaatcccgtgacttcgcggaatggccgatcatgaaggcgtcggaagcgattcaacgttatatctatgcactgtcgaccaccgaacgcgcggcctgtaaaccgggcaaaagtagtgagagccatgcagcgtggttcgccgctaccggtgtcagcaaccacggctacagccacgttcagggtctgaacctgatttttgatcataccttgggccgttacgatggtgttctgaaaaaggtccaactgcgcaacgaaaaggcgagagcgcgtttagagagcattaacgcgtctagagcagatgaaggtttaccggagattaaagccgaggaggaagaagtggctacgaacgaaaccggccacctgttgcaaccgccgggtatcaatccgagcttttacgtgtatcagaccattagcccgcaggcataccgtccgcgtgacgagatcgtgctgccgccggagtacgcaggttacgtacgtgacccgaatgctccgattccgctgggtgtggtgcgtaaccgttgcgatatccaaaaagggtgcccgggctacatcccggaatggcagcgtgaagcaggtaccgcgatcagcccgaaaacaggcaaggccgtaactgtgccgggcctgtctccgaagaaaaacaaacgtatgcgtaggtactggcgctctgagaaggagaaagctcaagatgcgctgttggttaccgtacgcattggtaccgattgggttgtcatcgacgtgcgcggtctgttgcgcaacgcccgttggcgcaccatcgctcccaaggacatcagcctgaatgcgctcctcgatttgtttaccggtgatccggtgatcgatgtgcgtcgcaatattgttacctttacgtacaccctcgacgcctgtggtacctacgcccgtaaatggaccttgaaaggcaaacagacgaaagcgaccctggacaaactgacggccactcagacggttgcgttggttgcgattgcgcttggtcagaccaacccgatttccgctggcattagccgtgttacacaagaaaatggtgcactgcaatgcgaaccgctggaccggttcaccctgccggatgacctgttgaaggacatcagcgcatatcgcattgcttgggacagaaacgaagaggaactacgcgctcgcagcgtcgaggccctgccggaggcgcaacaagcggaggtccgtgcgctggacggggtgtccaaagaaaccgcgcgtacccaactgtgcgcagatttcggcctggacccgaaacgtctgccgtgggataaaatgagctccaatactacctttatcagcgaagccttgctgtctaacagcgtgagccgtgatcaggtttttttcactccggctccgaagaagggtgctaagaaaaaagcgcctgttgaagttatgcgtaaagaccgcacttgggcacgtgcgtacaagccgcgtctgagcgtcgaagctcagaaactgaagaacgaagccctgtgggcacttaagcgcacctccccggagtatttgaagctgagccgtcgcaaagaggagctgtgtcgtcgtagcatcaactatgttatcgaaaagacccgtcgccgtacgcaatgtcagattgtgatcccggtgatcgaggatctgaatgttcgcttctttcatggttctggtaagagactgcccggttgggataatttcttcaccgctaaaaaagagaaccgttggttcatccagggcctgcacaaagcgtttagcgatctgcgtacgcaccgttccttctatgtttttgaagttcgtccggagcgcacctctattacctgtccaaagtgcggtcattgcgaggtgggtaatcgtgacggtgaggcgtttcagtgcctgagctgcggcaagacgtgcaacgcagatcttgacgtcgcgacccataatctgacccaagtggcgttgaccggtaagaccatgccgaagcgcgaggagccgcgtgacgctcagggtacagcgccagcgcgcaagactaagaaggcgagcaaatcaaaggcgccaccggcggagcgcgaagaccagaccccggcgcaagagccaagccagaccagttctggaggtagcgggggtagcggtggtagcactaacttaagtgatattatcgagaaagaaaccgggaaacagctggtgatccaagaaagcattctgatgctgcccgaagaagttgaagaagtgattggtaacaagcctgagtcggacatcttagtgcacaccgcgtacgatgaaagcacggatgagaatgtgatgctgctcacctctgatgcccccgaatacaagccgtgggctctggtgatccaggactctaacggagaaaacaaaattaagatgctctctggcggctccatggcgcctaaaaaaaaacgcaaagtggggattcacggggttccggcgtaa

SEQ ID NO: 60 - DNA, 5022 bp, synthetic construct
  source 1..5022: mol_type = other DNA
atgggaagtcatcaccaccaccaccattcaggtgagaacctgtacttccagagcatggctccgaaaaagaaacgtaaggtgggtatccacggcgttccggcgatggatccacagaggctaagacaatggcccggtccgggtccggcgagccgcggtggctacggccagcgtccgcgtatccgcaatccggaagagtggttccacgaactgagtccgagaacctttagcttccactttcgtaacctgcgttttgctagcggtcagaatcgcagctatatctgctgccaggtggaaggcaagaactgcttcttccaaggtatttttcaaaaccaggttccgccggacccaccttgtcatgctgaactttgctttttgtcctggttccaaagctggggtttgtcgccggacgagcactattacgtgacctggttcatctcctggtccccgtgctgtgaatgtgcggcgaaagtcgcccaatttctggaagagaaccgcaacgtgagcttgtctttgtctgcggcgcgtctgtactacttctggaaaagcgagtcccgtgagggcctgcgccgtctgtccgatctgggtgcacaggttggtattatgtcgttccaagattttcagcattgttggaataactttgttcataatctgggcatgccgttccagccgtggaagaaattgcacaaaaactaccaacgtctggtgaccgaattaaaacagattctccgcgaggagccggctacctatggctctccacaggcgcaaggtaaggtgcgcatcggcagcaccgcggcgggtctgcgtcacagccatagccacacccgtagcgaggcgcatctgcgtcccaaccactcatctcgtcagcaccgtatcctgaatccgccgcgcgaggcccgtgcgcgtacgtgcgttctggtagacgcatcctggatttgctatcgttctggaagtgagacacccgggacttcagaatccgcgaccccggagtctggaatgtcaaaactagaaaagtttacaaattgttactccctgtcgaaaaccctgagattcaaagcgatcccggtgggtaaaactcaagagaacattgataacaaacgtctgctggttgaggacgaaaagcgcgcggaggattacaaaggcgttaaaaagctgttggaccgttattacctgtcctttattaatgatgttctgcacagcattaaattaaagaacctaaacaattacatctccctcttccgtaaaaagacccgcaccgaaaaggaaaataaagaactggagaaccttgagattaatctgcgtaaagaaatcgctaaagcgtttaaaggcaacgagggttataaatccttatttaaaaaagacatcatcgaaacgattctcccagaattcctggacgacaaagacgagatcgcgctggttaattccttcaacggctttaccaccgcattcaccggcttttttgataaccgcgaaaacatgttctctgaagaggccaaaagcacctcaattgcgtttcgctgcattaacgaaaatctgacgcgttatatctctaacatggatatcttcgagaaggttgacgctatctttgataaacatgaagtccaggagatcaaggagaagatcctgaacagcgactacgacgtggaggatttcttcgagggtgagttcttcaattttgttttgacccaagaaggcatcgatgtctacaatgcgataattggtggttttgttaccgagagcggggagaaaattaagggtctgaacgaatatatcaacttgtacaaccagaaaaccaaacaaaaactgccgaagtttaagccgctgtacaagcaagtcttgtcggatcgtgaatcgctgagcttttacggcgaaggctacaccagcgatgaggaggtgttggaggtgttccgcaatacgttgaataagaactctgaaattttttccagcattaagaagctggaaaaactattcaagaacttcgacgagtatagcagcgcaggtatctttgtgaaaaacggtccggctattagcaccatctcgaaagacattttcggcgaatggaatgttattcgtgataagtggaatgccgaatacgacgacattcatctgaagaaaaaggcggttgtaactgaaaagtacgaggacgatcgtcgtaagtcgttcaaaaagatcggctccttctctctggagcaactgcaagagtacgccgatgccgatttgtcagtcgtggagaagctgaaagagatcatcattcaaaaagttgacgaaatctataaggtctatggtagcagtgaaaaactgtttgacgcggacttcgtgctggagaaaagcctgaagaaaaatgacgccgtggtggcaatcatgaaagatcttctggatagcgtcaagtctttcgaaaactacatcaaagcgtttttcggtgaaggcaaggaaacgaatcgtgacgagagcttctatggtgatttcgtcctagcctatgatatcctgcttaaggtggatcacatctatgatgccatccgcaactatgttacccagaaaccgtactcaaaggacaagttcaagttatacttccagaatccgcagtttatgggtggctgggataaagataaggaaaccgactatcgcgccaccattttacgttacggtagtaagtactatctggcaatcatggataaaaaatatgcgaaatgcctgcaaaagatcgacaaggacgatgttaacggcaattatgagaagatcaactacaaactgctgccgggcccaaataagatgctgccgaaggtgtttttcagcaaaaaatggatggcgtattacaatccgagcgaggacatccaaaagatctacaaaaacggtacgtttaagaagggtgatatgttcaacctcaacgattgtcataaacttattgacttcttcaaggatagcatatctcgttatccgaagtggagcaacgcgtatgactttaactttagcgaaaccgagaaatacaaagatatcgcgggtttctatcgtgaagttgaagaacagggttacaaagtgtccttcgagtccgcgagcaagaaggaggtagacaagctggtcgaggagggtaagttgtacatgtttcagatttacaataaggacttttccgacaagtcccatggtacaccgaatttgcacaccatgtatttcaagcttctgttcgacgagaacaaccacggccagatccgtctgagcggtggtgcagagctgttcatgcgtagagctagcctgaagaaagaagagttggtcgttcatccggctaattcgccgatcgcgaacaaaaatccagataaccctaaaaagactacgaccctgagctatgatgtgtataaagacaaacgctttagcgaagaccagtatgagttacacattccgatcgctatcaacaagtgcccgaagaatattttcaagatcaacaccgaagtgcgtgttcttttgaagcacgatgacaacccgtatgtgattggtattgctcgtggtgaacgtaatctgctgtacattgtggttgtggacggcaaaggaaacatcgtggaacagtacagcctgaacgaaatcattaacaatttcaacggcattcgcattaagacggattaccattctttgctggacaaaaaggagaaagagcgtttcgaggcccgtcagaactggacctccatcgagaacattaaggagctgaaggcgggttacatcagccaggtggtgcacaaaatctgcgaactggtggagaagtacgacgcggtgattgctttggcggatttgaactccggttttaagaacagccgcgttaaagtggaaaagcaggtttatcagaaattcgaaaaaatgctgattgataaattgaattacatggttgacaaaaagtctaacccgtgtgcaaccggcggtgcgctgaagggctaccaaatcaccaataagtttgaaagctttaagagcatgagcacgcaaaatggcttcattttttacattccggcgtggctgaccagcaagatcgatccgtcaaccggctttgttaatttactgaagacgaagtatacctctattgccgactctaaaaaatttattagttccttcgatagaatcatgtatgttccggaagaggacctgttcgaatttgcactggactacaagaactttagccgtaccgatgctgattatataaagaaatggaaactctacagctatggcaaccgcatccgtatctttcgcaacccgaaaaaaaacaacgtgttcgactgggaagaggtatgtctgacttcagcgtacaaggagcttttcaataaatatggtatcaactaccagcagggtgatatccgtgcactgctttgcgagcaaagcgataaagcgttctattcaagcttcatggcgctgatgagcctgatgctgcaaatgcgcaacagcatcaccggtcgtaccgatgttgcatttctgatttctccagtaaaaaacagcgatggtatcttttacgacagccgtaattacgaggcccaagaaaatgcgattttgccgaagaacgctgacgcgaatggtgcatacaacattgcaaggaaagttctgtgggcaatcggccaatttaaaaaagcggaagatgagaagctggacaaagtgaaaattgcgattagcaacaaggagtggctggagtatgcgcagacctccgttaaatcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttaa

SEQ ID NO: 61 - DNA, 33 bp, synthetic construct
  source 1..33: mol_type = other DNA
atgggaagtcatcaccaccaccaccattcaggt

SEQ ID NO: 62 - DNA, 60 bp, synthetic construct
  source 1..60: mol_type = other DNA
tctggcggtagcatggctccgaaaaagaagcgtaaagtaggtattcatggtgttccggca

SEQ ID NO: 63 - DNA, 30 bp, synthetic construct
  source 1..30: mol_type = other DNA
tcagggggaagtggcggaagcggtgggtct

SEQ ID NO: 64 - DNA, 48 bp, synthetic construct
  source 1..48: mol_type = other DNA
atggctccgaaaaagaaacgtaaggtgggtatccacggcgttccggcg

SEQ ID NO: 65 - DNA, 21 bp, synthetic construct
  source 1..21: mol_type = other DNA
gagaacctgtacttccagagc

SEQ ID NO: 66 - DNA, 249 bp, synthetic construct
  source 1..249: mol_type = other DNA
acgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttg

SEQ ID NO: 67 - DNA, 45 bp, synthetic construct
  source 1..45: mol_type = other DNA
tctggaagtgagacacccgggacttcagaatccgcgaccccggag

SEQ ID NO: 68 - DNA, 750 bp, synthetic construct
  source 1..750: mol_type = other DNA
atgggaagtcatcaccaccaccaccattcaggtgagaacctgtacttccagagcatggctccgaaaaagaaacgtaaggtgggtatccacggcgttccggcgatggaagcaagtccagcttcaggacccaggcacctgatggacccgcatattttcacctctaattttaacaacggtattggccgtcacaagacgtacctgtgttatgaagtggaacgtcttgacaacggcacctccgtgaaaatggaccagcatcgtggtttcttgcataatcaagctaaaaacctgctgtgcggtttctacggccgtcacgcggaactgcgttttttggacctggttccgagcctgcaactggacccggcacagatttatcgtgttacttggttcatcagctggtcgccgtgttttagctggggttgcgccggcgaggtacgcgcgtttttgcaagagaacacccacgtgcgtttgcgcatcttcgccgctcgtatctacgattatgacccactgtataaagaagcgctccagatgctgcgcgatgcaggcgcgcaggtttccatcatgacctacgacgagttcaagcactgctgggatacctttgtcgatcatcagggctgcccgttccagccgtgggatggtctggatgagcacagccaggcgttaagcggtcgcctcagagcgattctgcaaaaccaaggtaattctggaagtgagacacccgggacttcagaatccgcgaccccggagtctgga

SEQ ID NO: 69 - DNA, 750 bp, synthetic construct
  source 1..750: mol_type = other DNA
atgggaagtcatcaccaccaccaccattcaggtgagaacctgtacttccagagcatggctccgaaaaagaaacgtaaggtgggtatccacggcgttccggcgatggaagcatcaccagctagtggacccaggcacctgatggatccgcacatcttcacctccaattttaataacggcatcggccgtcataagacgtacctgtgctatgaggttgaacgtttggacaacggtactagcgtcaagatggaccagcatcgtggtttccttcacaaccaagcgaaaaatctgctgtgcggtttttatggtcgtcacgccgaactgagattcttggatctggttccgtctctgcaactggacccggcgcagatttatcgtgtgacctggttcatcagctggtcgccgtgctttagctggggttgcgccggtgaagtgcgcgcatttctccaggagaacacccatgttcgtttgcgcatcttcgcggcacgtatcttcgattacgacccgctgtacaaggaggcgctgcaaatgttgcgtgatgctggcgctcaagttagcattatgacctacgacgaatttaaacactgttgggataccttcgtggatcatcagggctgtccgtttcagccatgggacggcctggacgagcacagccaggcgttatccggtcgcctgcgcgcgattctgcaaaaccagggaaactctggaagtgagacacccgggacttcagaatccgcgaccccggagtctgga

SEQ ID NO: 70 - DNA, 732 bp, synthetic construct
  source 1..732: mol_type = other DNA
atgggaagtcatcaccaccaccaccattcaggtgagaacctgtacttccagagcatggctccgaaaaagaaacgtaaggtgggtatccacggcgttccggcgatggaaatactaaggtatttaatggatcccgacactttcaccttcaatttcaacaacgatccattggtcttgcgccgtcgtcagacctacttgtgctatgaggttgaacgtctggataacggtacgtgggttttaatggatcagcatatgggtttcctgtgtaatgaagcgaagaacctgctgtgcggtttttacggccgtcatgcggaactgcgttttcttgacctggttccgagcctgcaactggacccggctcagatttaccgtgttacctggtttatctcgtggagcccgtgttttagctggggctgcgcgggtgaagtgcgcgcgtttttgcaagagaacacccacgtgcgtctccgcatcttcgccgcacgtatttacgattatgacccgctgtataaagaggccctgcaaatgctgagagatgctggcgcacaagtgagcattatgacctacgacgagttcgagtactgctgggatacgttcgtgtatcgccaaggttgcccgtttcagccgtgggacggcctggaggagcacagccaggcgctctccggtcgtttgcgcgcgatcctgcaatctggaagtgagacacccgggacttcagaatccgcgaccccggagtctgga

SEQ ID NO: 71 - DNA, 747 bp, synthetic construct
  source 1..747: mol_type = other DNA
atgggaagtcatcaccaccaccaccattcaggtgagaacctgtacttccagagcatggctccgaaaaagaaacgtaaggtgggtatccacggcgttccggcgatggattcactattaatgaataggagaaaattcctgtatcagtttaagaacgtccgttgggctaaaggtcgtcgtgagacgtatttatgctacgtggtgaaacgtcgcgacagcgcaacctccttcagcctggacttcggctatctgcgtaacaaaaacggctgccacgtggagctgctgtttttgcgctacattagcgactgggatttggacccaggtcgctgctatcgtgttacctggtttacgagctggtcgccgtgttacgactgtgcgcgtcatgtcgcggattttctgcgcggtaatccgaatctgtccctgcgtatttttaccgcacgtctctacttctgcgaagatcgtaaggcggaaccggaaggcttgcgtcgcctgcatagagcgggtgttcagatcgctatcatgaccttcaaagattatttctactgctggaataccttcgtggagaaccacgagcgtactttcaaggcctgggaaggccttcacgaaaacagcgttcgcttgtctcgtcaactgcgccgcatcctgctgccgctgtacgaggttgatgatctcagagacgcgtttcgtaccctgggtttgtctggaagtgagacacccgggacttcagaatccgcgaccccggagtctgga

SEQ ID NO: 72 - DNA, 729 bp, synthetic construct
  source 1..729: mol_type = other DNA
atgggaagtcatcaccaccaccaccattcaggtgagaacctgtacttccagagcatggctccgaaaaagaaacgtaaggtgggtatccacggcgttccggcgatggctgattcaagtgaaaaaatgaggggacaatatatcagccgtgacacctttgagaaaaactacaaaccgattgacggcacgaaagaggcgcatctgctgtgcgaaattaaatggggtaagtacggcaagccgtggctgcattggtgtcaaaaccaacgtatgaatatccatgctgaagactacttcatgaacaacatcttcaaagccaaaaagcatccggtgcactgctacgtgacctggtatttgtcctggtcaccgtgcgcggattgtgcgagcaagatcgttaagttcctggaggagcgtccttatctgaaactgaccatttatgttgcacagctgtattatcacactgaagaggagaaccgtaaaggtctgcgtttgctgcgttcgaagaaggtgattatccgcgtgatggatatctctgattacaactactgctggaaagtctttgttagcaatcagaacggtaatgaagactactggccgctgcaattcgatccgtgggttaaggagaactacagccgcctcttggacattttctgggaaagcaagtgccgttccccaaatccgtggtctggaagtgagacacccgggacttcagaatccgcgaccccggagtctgga

SEQ ID NO: 73 - DNA, 840 bp, synthetic construct
  source 1..840: mol_type = other DNA
atgggaagtcatcaccaccaccaccattcaggtgagaacctgtacttccagagcatggctccgaaaaagaaacgtaaggtgggtatccacggcgttccggcgatgagttcagaaacaggacccgttgctgtagacccgacgctgcgccgtcgtattgagccgcacgagttcgaggttttttttgatccgcgtgaactccgcaaggagacatgcttgctgtacgagatcaaatggggcacttcacacaaaatctggcgccacagcagcaagaacaccaccaagcacgtggaagtgaacttcatcgagaaattcacgtctgaacgtcacttttgtccgtccaccagctgtagcattacctggtttctatcctggagcccgtgcggtgaatgttccaaggcgattaccgaattcttgtcgcaacatccgaacgtgactcttgttatttatgtggcccgtttgtaccatcacatggatcagcagaatcgtcaaggcttgcgcgacctggttaacagcggtgttaccatccagatcatgaccgcaccggaatacgactactgctggcgtaatttcgtcaactatccgccgggcaaggaggcgcattggccgcgttatccgccgctgtggatgaaactgtacgctctggaactgcatgcgggtattttaggcctgccaccgtgcttgaatattctgagacgcaagcagcctcagctgacctttttcacgatcgcgctgcaaagctgtcattatcaacgtctgccgccacacatcctgtgggctaccggtctgaaatctggaagtgagacacccgggacttcagaatccgcgaccccggagtctgga

SEQ ID NO: 74 - DNA, 750 bp, synthetic construct
  source 1..750: mol_type = other DNA
atgggaagtcatcaccaccaccaccattcaggtgagaacctgtacttccagagcatggctccgaaaaagaaacgtaaggtgggtatccacggcgttccggcgatggaagcaagtcccgcttcaggaccaaggcacctgatggacccacatattttcaccagtaattttaacaacggcatcggccgtcacaagacctacctgtgttatgaggtcgaacgtctggataatggcacttccgtgaaaatggatcagcatcgtggtttcctgcatggtcaggctaagaatctgctttgcggtttctatggtcgccacgccgaactgcgttttttggacctggttccgtccctccaactggacccggcgcaaatctatcgtgttacctggtttatcagctggtcgccgtgtttcagctggggttgcgcaggcgaagtacgtgcgttcttacaagagaacacccatgtgcgcttgcgcatcttcgccgcacgtatttacgactacgacccgctgtataaagaggcgttgcaaatgctgcgcgatgctggcgcacaggttagcattatgacgtacgacgaatttaagcactgctgggataccttcgtggatcaccagggctgcccgtttcagccgtgggatggtttggacgagcactctcaggcgctgagcggtcgtctcagagcgattctgcaaaaccagggaaactctggaagtgagacacccgggacttcagaatccgcgaccccggagtctgga

SEQ ID NO: 75 - DNA, 909 bp, synthetic construct
  source 1..909: mol_type = other DNA
atgggaagtcatcaccaccaccaccattcaggtgagaacctgtacttccagagcatggctccgaaaaagaaacgtaaggtgggtatccacggcgttccggcgatgtcaagtaaaacaggacccgttgctgtagatccgacgctgagacgtcgcatcgagccgcacgaattcgaggtgttttttgatccgcgtgaactgcgcaaggagacgtgcctgctgtatgagatcaactggggtggccgtcacagcatttggcgtcacacctcccaaaacacgaacaaacacgtggaagtcaatttcatcgaaaaattcaccaccgaacgttacttctgcccgaacacccgttgttccatcacctggttcctgtcttggagcccgtgtggtgagtgcagccgcgcaattactgagttcctgagccgttacccgaatgttaccctgtttatctatatcgcccgtttgtaccatctggcgaacccgcgcaaccgtcagggtttacgcgacttaattagctctggcgttaccattcagattatgaccgagcaagagagcggttattgctggcataactttgtaaactatagcccgagcaatgaaagccattggcctcgctacccgcacctgtgggttcgtttgtacgtgctggaactgtactgcatcatccttggtttgccaccgtgcttgaatattttgagacgtaagcagtcgcaactgaccagttttacgatcgcgctacagagctgtcattatcagcgtctcccgccacacattctgtgggctaccggcctgaaaagcggcggtagcagcggtggctccagcggctcggaaactccgggcacctccgagtcagcgaccccggaatctggaagtgagacacccgggacttcagaatccgcgaccccggagtctgga

SEQ ID NO: 76 - DNA, 849 bp, synthetic construct
  source 1..849: mol_type = other DNA
atgggaagtcatcaccaccaccaccattcaggtgagaacctgtacttccagagcatggctccgaaaaagaaacgtaaggtgggtatccacggcgttccggcgatgtcaacagatgctgaatatgtaaggatacacgagaagctggacatctacacctttaaaaagcagttctccaacaataaaaaatctgtcagccatcgttgctacgtgttgtttgaactcaagcgtcgtggcgaacgccgtgcgtgcttttggggttacgctgtgaacaaaccgcagagcggtacggaacgtggtattcatgcggagatcttcagcatccgcaaggtggaagagtatctgcgcgataacccgggtcaattcaccattaactggtacagcagctggtcaccgtgcgccgactgtgctgagaagattttggagtggtataaccaagaattgcgcggtaatggccatactctgaagatctgggtgtgtaaactttattacgaaaagaacgcgcgtaatcaaattggtctgtggaatctgagagataacggcgttggtctgaatgttatggtttccgagcactatcagtgctgccgtaaaatcttcattcagtccagccacaaccaactgaacgagaaccgttggctggaaaagaccctgaagcgcgcggagaaacgtcgtagcgagctgagcattatgtttcaggttaaaatcttacacaccaccaaatcgccggcagtctctggcggttcctcgggcggcagctctggctccgaaaccccaggtacgagcgagagcgcaaccccggaatctggaagtgagacacccgggacttcagaatccgcgaccccggagtctgga

SEQ ID NO: 77 - DNA, 711 bp, synthetic construct
  source 1..711: mol_type = other DNA
atgggaagtcatcaccaccaccaccattcaggtgagaacctgtacttccagagcatggctccgaaaaagaaacgtaaggtgggtatccacggcgttccggcgatgtcatttgagaggaattatgatcccagagaattgcgcaaagagacgtacctgctgtatgaaatcaagtggggtaaaagcggcaagctgtggcgtcattggtgtcaaaacaaccgcacccaacacgcggaagtttatttcctcgagaatattttcaacgcccgtcgttttaacccgagcacccactgctccatcacctggtatctttcttggtcgccgtgtgcggagtgctcgcaaaaaatcgtggacttcctgaaagaacatccgaatgtgaacctggagatctatgttgcgagattgtactatccggagaacgaacgtaatcgtcagggcctgcgcgacttggtcaacagcggggtgaccattcgtattatggatctgccggattacaattactgctggaaaacctttgtttccgatcagggtggcgacgaagactactggcctggtcactttgctccgtggattaagcagtacagcctgaagctgagcggtggttcttccggcggtagcagtggctccgagactccaggtacgagcgagagcgcaaccccggaatctggaagtgagacacccgggacttcagaatccgcgaccccggagtctgga

SEQ ID NO: 78 - DNA, 717 bp, synthetic construct
  source 1..717: mol_type = other DNA
atgggaagtcatcaccaccaccaccattcaggtgagaacctgtacttccagagcatggctccgaaaaagaaacgtaaggtgggtatccacggcgttccggcgatggatcccccaacatttactttcaactttaataacgaaccgtgggttcgcggtcgccacgaaacctacctgtgctatgaggtggaacgtatgcataatgatacttgggttctgctgaatcaacgtagaggctttttatgcaaccaagctccgcataaacatggttttctggaaggccgtcacgccgagctgtgcttccttgacgtgatcccattctggaaactggacttggaccaggattatcgtgtgacctgtttcaccagctggtcgccgtgcttcagctgtgcgcaggagatggcgaagttcatctccaaaaacaagcacgttagcttgtgcatttttaccgcgcgtatttacgatgatcaaggtcgctgccaagagggcctgcgtaccctggcagaggctggtgcgaagatctctatcatgacgtacagcgaatttaaacactgctgggacacgtttgtcgatcaccagggttgtccgttccagccgtgggacggcttggacgaacatagccaggatttgtccggtcgtctgcgcgcaattctacagaatcaagagaactctggaagtgagacacccgggacttcagaatccgcgaccccggagtctgga

SEQ ID NO: 79 - DNA, 108 bp, synthetic construct
  source 1..108: mol_type = other DNA
atgggcagccaccaccaccaccaccacagcggcgagaacctgtacttccagtccatggctccaaagaagaagagaaaggttggaatccacggagttccagcttctgga

SEQ ID NO: 80 - DNA, 495 bp, synthetic construct
  source 1..495: mol_type = other DNA
atgggaagtcatcaccaccaccaccattcaggtgagaacctgtacttccagagcatggctccgaaaaagaaacgtaaggtgggtatccacggcgttccggcgagcggattagtaatgcaaaattataggctaatagacgctacgttgtatgtgacctttgaaccgtgcgttatgtgcgctggtgcaatgattcattctcgtatcggccgtgtcgtgttcggcgtgagaaacagcaagcgcggtgcggcgggtagcctgatgaatgttctgaactacccgggtatgaaccaccgtgttgaaattaccgagggtatcttggcagacgagtgcgccgccctgctgtgtgatttctaccgcatgccgcgtcaggtttttaacgcgcagaaaaaggcgcaaagcagcatcaatagcggcggtagctctggcggctcctccggttcggagtctggaagtgagacacccgggacttcagaatccgcgaccccggagtctgga

SEQ ID NO: 81 - DNA, 777 bp, synthetic construct
  source 1..777: mol_type = other DNA
atgggaagtcatcaccaccaccaccattcaggtgagaacctgtacttccagagcatggctccgaaaaagaaacgtaaggtgggtatccacggcgttccggcgatgacagatgctgaatatgtaaggatacacgagaagttggacatctacacctttaaaaagcagttctttaacaacaaaaagagcgtcagccatcgttgctatgttctgtttgagttgaagcgccgtggcgaacgtcgtgcgtgcttttggggttatgctgtgaacaaaccgcagtctggtactgaacgcggtattcatgcagaaattttcagcattcgtaaggtggaagaatacctgcgcgataacccaggtcagttcaccattaactggtacagcagctggtccccgtgcgcggattgcgctgagaagatcttggagtggtacaatcaagagctgcgtggtaatggccataccctgaaaatctgggcgtgcaaactgtattacgaaaagaacgcgcgtaatcaaattggtctgtggaatctgcgtgacaacggcgtgggcctgaatgttatggtttccgagcactatcagtgttgtcgtaaaatcttcatccaatcctctcacaaccaattgaacgagaaccgctggctggagaagaccctgaagcgcgccgagaaaagacgtagcgaactctcgatcatgattcaggttaaaatcttacacacgaccaaaagcccggcagtgtctggaagtgagacacccgggacttcagaatccgcgaccccggagtctgga

SEQ ID NO: 82 - DNA, 861 bp, synthetic construct
  source 1..861: mol_type = other DNA
atgggaagtcatcaccaccaccaccattcaggtgagaacctgtacttccagagcatggctccgaaaaagaaacgtaaggtgggtatccacggcgttccggcgatgacaagtgaaaaaggaccctcaactggggacccaaccctgcgtcgtcgcattgaaagctgggagttcgacgttttttatgatccgcgtgagctggcgaaagagacgtgcctgttgtacgagatcaaatggggtatgagccgtaaaatctggagatccagcggtaaaaacaccaccaatcacgtggaagtgaattttatcaagaagttcaccagcgaacgtcgtttccacagctccatttcttgttctattacctggtttctgtcttggtcgccgtgttgggagtgctcccaggcaattcgtgaattccttagtcaacatccgggcgtgaccctggtcatctacgtcgcccgtttgttttggcatatggaccagcgcaatcgtcagggtttgcgcgatctggttaacagcggcgttacgatccaaattatgcgtgcgagcgagtattaccactgctggcgcaacttcgtgaactatccaccgggcgatgaagcacattggccgcaatatccgccgctgtggatgatgctgtacgcgctggaactccactgcattatcctgtcgctgccgccttgtctgaagatcagccgacgctggcagaaccacctcgcgtttttccgcctgcacttgcaaaactgccattaccagaccatcccgccgcatatcctgttagctaccggtctgattcacccgagcgttacttggcgttctggaagtgagacacccgggacttcagaatccgcgaccccggagtctgga

SEQ ID NO: 83 - DNA, 840 bp, synthetic construct
  source 1..840: mol_type = other DNA
atgggaagtcatcaccaccaccaccattcaggtgagaacctgtacttccagagcatggctccgaaaaagaaacgtaaggtgggtatccacggcgttccggcgatgagttcagaaacaggacccgttgctgtagacccgaccctgcgtcgccgcattgagccgcacgagttcgaagtcttttttgatccgcgtgaactgcgcaaagaaacctgtctgctgtatgaaattaactggggtggccgtcacagcatttggcgtcacactagtcaaaacaccaataaacacgtggaagttaacttcatcgagaaatttaccaccgagcgctacttctgcccgaatactcgttgttctattacgtggtttctctcttggtcgccgtgcggcgagtgcagccgtgcgattaccgagttcctgagccgttacccgcacgttaccctgtttatctatatcgcgcgtctgtaccatcatgcggacccgcgtaaccgtcaaggtctgcgtgatctgatcagcagcggtgttacgatccagattatgacggagcaggagtccggctattgctggcgcaacttcgtgaactatagcccatccaatgaagctcattggccgcgctacccgcacctgtgggtacgtttgtacgtgctggaactctactgcatcatccttggcctgccaccgtgcttgaatattttgagacgcaagcagccgcaactgaccttcttcaccatcgccctgcaaagctgtcattatcagcgtttgccgcctcacatcttatgggcaaccggtttgaagtctggaagtgagacacccgggacttcagaatccgcgaccccggagtctgga

SEQ ID NO: 84 - DNA, 840 bp, synthetic construct
  source 1..840: mol_type = other DNA
atgggaagtcatcaccaccaccaccattcaggtgagaacctgtacttccagagcatggctccgaaaaagaaacgtaaggtgggtatccacggcgttccggcgatgagttcagaaacaggacccgttgctgtagacccgaccttgcgcagacgtattgagccgcacgaattcgaggtgtttttcgacccgagagaattgcgcaaagaaacctgtctgctgtacgagatcaactggggtggtcgtcatagcatctggcgtcacacctctcagaatacgaacaaacacgtggaagtcaatttcatcgagaagttcacgaccgagcgctacttctgtccgaacacccgttgttcgatcacgtggtttctgagctattctccgtgcggcgaatgcagccgtgcaattaccgagttcctgtcacgttacccgcacgtaactctgtttatctacattgcgcgtctctaccatcatgctgatccggaaaaccgtcaaggtttacgcgatctgattagctccggcgtgaccatccagattatgactgagcaggagagcggctattgctggcgcaactttgttaactattccccgtccaatgaggcgcattggcctcgttatccgcacctgtgggttcgtctatacgttctggaattgtactgcattatcctgggcctgccaccatgcttgaatattctgcgtcgcaagcagccgcaattgacctttttcaccatcgccctgcaaagctgccattatcagcgccttccgccgcacatcctgtgggcgaccggtctgaaatctggaagtgagacacccgggacttcagaatccgcgaccccggagtctgga

SEQ ID NO: 85 - DNA, 840 bp, synthetic construct
  source 1..840: mol_type = other DNA
atgggaagtcatcaccaccaccaccattcaggtgagaacctgtacttccagagcatggctccgaaaaagaaacgtaaggtgggtatccacggcgttccggcgatgtcaagtgaaacaggacccgttgctgtagacccgaccttgagacgccgcattgagccgcacgaatttgaagttttcttcgacccgcgtgaattgcgtaaagagacgtgcttgctgtacgagattaactggggtggccgtcacagcatttggcgccacaccagccaaaacaccaacaaacatgtggaagtcaacttcatcgagaagttcactaccgagcgttacttttgtccgaatacgcgttgttcgatcacctggtttctgtcctattccccgtgcggtgaatgctcacgcgcaatcactgagttcctgtctcgttacccgcacgtaaccctgtttatctatatcgcgcgtttgtaccatcatgcggatccggagaaccgccagggtctggaagatttaattagcagcggcgttacgatccagattatgaccgagcaggagagcggctattgctggcgtaatttcgtgaactattccccgtctaatgaagcgcattggccgcgttacccacacctgtgggttcgtctctacgtgctggaattgtactgcattatcctgggtttaccgccttgcctgaatattctacgtcgcaagcaaccgcagctgacctttttcaccatcgccctgcaaagctgtcattatcagcgtctgccgccacacatcctgtgggctaccggccttaaatctggaagtgagacacccgggacttcagaatccgcgaccccggagtctgga

SEQ ID NO: 86 - DNA, 735 bp, synthetic construct
  source 1..735: mol_type = other DNA
atgggaagtcatcaccaccaccaccattcaggtgagaacctgtacttccagagcatggctccgaaaaagaaacgtaaggtgggtatccacggcgttccggcgatgaaacctcagattcgcgatcatcgtccgaatcctatggaggcgatgtatccgcatattttctactttcattttgaaaacttagaaaaagcctatggccgtaacgaaacttggctgtgctttaccgtagagattatcaaacaatatttaccggtaccctggaaaaaaggtgttttccgtaatcaggttgatccggagacacattgtcatgcagagaagtgcttcctgtcctggttctgcaataacacccttagcccgaaaaagaattaccaagttacttggtacacgagctggtcgccatgtccagagtgcgcaggagaagtagcagaattcttagctgaacattcgaacgtaaaattgacaatctacaccgcccgtctctactacctgtgggacaccgactatcaggaaggcctgcgctcattgtctgaggagggcgcatcggtcgaaatcatggattatgaagattttcagtattgttgggaaaatttcgtatacgacgatggtgagccttttaaacgttggaaaggcctgaagtacaactttcagtctttgacccgtcgcttgcgcgaaattctgcaatctggaagtgagacacccgggacttcagaatccgcgaccccggagtctgga

SEQ ID NO: 87 - DNA, 993 bp, synthetic construct
  source 1..993: mol_type = other DNA
atgggaagtcatcaccaccaccaccattcaggtgagaacctgtacttccagagcatggctccgaaaaagaaacgtaaggtgggtatccacggcgttccggcgatggatccacagaggctaagacaatggcccggtccgggtccggcgagccgcggtggctacggccagcgtccgcgtatccgcaatccggaagagtggttccacgaactgagtccgagaacctttagcttccactttcgtaacctgcgttttgctagcggtcagaatcgcagctatatctgctgccaggtggaaggcaagaactgcttcttccaaggtatttttcaaaaccaggttccgccggacccaccttgtcatgctgaactttgctttttgtcctggttccaaagctggggtttgtcgccggacgagcactattacgtgacctggttcatctcctggtccccgtgctgtgaatgtgcggcgaaagtcgcccaatttctggaagagaaccgcaacgtgagcttgtctttgtctgcggcgcgtctgtactacttctggaaaagcgagtcccgtgagggcctgcgccgtctgtccgatctgggtgcacaggttggtattatgtcgttccaagattttcagcattgttggaataactttgttcataatctgggcatgccgttccagccgtggaagaaattgcacaaaaactaccaacgtctggtgaccgaattaaaacagattctccgcgaggagccggctacctatggctctccacaggcgcaaggtaaggtgcgcatcggcagcaccgcggcgggtctgcgtcacagccatagccacacccgtagcgaggcgcatctgcgtcccaaccactcatctcgtcagcaccgtatcctgaatccgccgcgcgaggcccgtgcgcgtacgtgcgttctggtagacgcatcctggatttgctatcgttctggaagtgagacacccgggacttcagaatccgcgaccccggagtctgga

SEQ ID NO: 88 - DNA, 4449 bp, synthetic construct
  source 1..4449: mol_type = other DNA
atggataaaaagtattcaataggactagctattggcaccaacagcgttggttgggcagttatcaccgatgaatataaggtcccgagcaagaagtttaaggtcctgggcaacaccgatcgccacagcataaaaaagaaccttattggtgcgctgctgttcgattctggtgagactgccgaagcgacccgtctgaagagaaccgcgcgtcgtagatacacccggcgcaagaaccgtatctgctatctgcaagaaattttcagcaacgagatggctaaggtcgacgactccttctttcatcgcctggaggaaagctttctggttgaggaagacaaaaagcacgagcgtcatccgattttcggtaacattgttgatgaagttgcctaccacgaaaaataccccacaatttatcatctgcgtaaaaaactggtagactccaccgataaggcggatttacgccttatctatttggcgctggcgcatatgattaagttccgcggtcattttttgatcgagggcgacctgaatccggataacagcgacgttgacaaactgtttatccagctggtgcagacctataaccagttgttcgaagagaacccgattaatgcgtctggcgtggacgcgaaggcgatcctgagcgctcgtctgtccaagtcacgccgcctggagaacttgatcgcgcaactgccgggtgagaagaaaaacggcttgttcggcaatctgattgctctgtccctaggtttgaccccgaatttcaaatctaattttgatctggcggaagatgcgaaattacagttgagcaaggacacttacgatgacgatctggacaacctgctcgctcagatcggcgatcagtatgccgatcttttcctggccgcgaagaacctgagcgatgccatcttgctatcggacatcttacgtgttaacaccgaaatcaccaaagctccgctaagcgcgtccatgattaaacgttacgatgaacaccaccaggatctgacgctgctgaaagcgttggtgcgccagcaactgccggaaaaatacaaggaaattttcttcgaccaaagcaagaacggttatgcaggctatatcgacggtggtgcgtcgcaagaagaattttataaattcatcaaaccgattttggagaagatggatggtaccgaggagctgctggtaaaattgaatcgggaagacctcttgcgaaagcagcgtactttcgataatggtagcatcccgcaccagattcatctgggcgaacttcacgcaatcctacgtcgtcaagaggatttctacccgtttctgaaggataaccgtgaaaagatcgagaagattctgacctttcgtattccgtattatgtgggtccgctggcgagaggcaacagccgttttgcgtggatgacccgcaagtcggaagagactatcacgccgtggaatttcgaggaggtcgtggacaagggtgcatcagcgcaatcttttattgaacgtatgaccaattttgataaaaaccttccaaatgaaaaagtgttgccgaagcacagcttgctgtacgagtatttcaccgtttacaatgaactgacgaaagtcaagtacgttaccgaaggtatgcgtaagccagcctttctgtctggggagcaaaaaaaagccattgtggatctgctgttcaagaccaatcgcaaggttaccgtcaagcaactgaaagaggattacttcaaaaaaatcgaatgttttgactccgtggaaatcagcggcgttgaagaccgctttaatgcatctctgggcacctaccacgatctactcaagatcatcaaggataaggacttcctggacaacgaagaaaatgaggatatcttggaagatattgtgctgactttaacgttgttcgaggatcgtgagatgattgaagaacgcctcaaaacgtacgcacatctgtttgacgataaggtgatgaaacagctaaagcgccgtcgttacaccggttggggtcgtctgtctagaaagctgattaacggcattcgtgacaaacaatctggcaagaccatcctggacttcctcaaatccgacggcttcgcaaatcgtaatttcatgcaactgatccatgatgatagcctgacctttaaagaagatattcagaaggctcaagtgtccggccagggtgactcgctgcatgagcatatcgcgaatctggctggctcccctgcgataaaaaaaggtattctccaaacggtgaaagttgtggatgaactggttaaagtcatgggtcgtcataaaccggaaaacatcgtgattgagatggcacgtgagaaccagaccacccagaaaggtcaaaaaaacagtcgtgagcgtatgaaacgtatcgaagaggggattaaggagttaggcagccagattctgaaggagcacccggttgagaacacccagctgcaaaacgagaagctctacctgtattacctgcaaaacggccgtgacatgtatgttgaccaggagctggacatcaatcgtctcagcgactacgacgtggatgctatcgtgccgcagagctttctaaaagatgattccatcgacaataaagtgctcacccgtagcgacaagaaccgtggtaaaagcgacaacgtgccgagcgaggaagtagttaagaaaatgaaaaactactggcgtcagctattgaacgccaagcttatcacccagcgtaagtttgacaacttgacgaaggcagagcgtggcggcctgagcgagctagacaaggcgggttttattaaacgccagctggtggaaactcgccagattaccaaacacgttgcgcaaatcttggactcacgcatgaatacgaagtacgacgaaaacgataaattgatccgcgaggtgaaggtcataaccctgaaatctaaactggttagcgatttccggaaggactttcaattttataaagtccgggagattaacaactatcaccatgcacacgacgcttacttaaatgcggttgttggcacggccctgatcaagaagtacccgaaactggaaagcgagtttgtttatggtgactacaaggtgtatgacgttcgcaagatgattgcaaaatccgaacaagagattggtaaggcgacggctaagtattttttctacagcaatatcatgaacttcttcaagactgaaatcaccctggcgaacggtgaaattcgtaaacgtccgttgatcgaaaccaacggtgaaaccggtgaaatcgtttgggataaaggtagggattttgcgaccgttcgtaaggtcctgagcatgccgcaggttaacatcgtgaaaaagaccgaagtgcagaccggcggtttctccaaggagagtatcttaccgaagcgcaacagtgacaagctgatcgcgcgtaaaaaagactgggatccgaaaaaatacggcggctttgactccccgaccgtcgcgtacagcgtactggtcgtggctaaagtggagaaaggcaaatcgaaaaagctgaaatccgttaaagagttgctgggtattacgattatggaacgttcgagcttcgagaaaaatccgatcgacttcctggaggccaaaggctacaaggaagttaaaaaagacctgatcattaaactgccgaagtacagcctgttcgagctcgagaacggtcgcaaacgcatgctggcgagcgcgggtgagctccaaaaaggcaacgaattggctctgccgagcaagtacgtgaacttcctgtatctggcctcccattatgaaaaactgaagggtagcccggaagataacgaacaaaagcagctgttcgtggaacaacataaacactatctggacgagattattgagcaaatcagcgagttcagcaaacgcgtgattctggcggatgctaatctggacaaggttctttcagcctataataaacaccgtgataagccgatccgtgaacaggcagaaaatattattcacctgttcaccctgacgaacctgggtgcgccagcagcgtttaaatacttcgacacaacgatcgaccggaagcgttataccagcaccaaagaggttcttgacgccaccttgattcaccaaagcatcacgggtctgtatgaaacccgtattgacttgagccagttgggcggagattcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggt

SEQ ID NO: 89 - DNA, 2616 bp, synthetic construct
  source 1..2616: mol_type = other DNA
atgccaaaacccgctgtagagtcagaattttcaaaagttttgaaaaagcactttccgggagaacgtttccgctcgagctatatgaaaagaggcggtaagatcctggctgcccaaggcgaagaggcagttgttgcgtatctacagggcaagtccgaggaggagcctccgaacttccagccaccggcgaagtgccacgtggtgaccaaatcccgtgacttcgcggaatggccgatcatgaaggcgtcggaagcgattcaacgttatatctatgcactgtcgaccaccgaacgcgcggcctgtaaaccgggcaaaagtagtgagagccatgcagcgtggttcgccgctaccggtgtcagcaaccacggctacagccacgttcagggtctgaacctgatttttgatcataccttgggccgttacgatggtgttctgaaaaaggtccaactgcgcaacgaaaaggcgagagcgcgtttagagagcattaacgcgtctagagcagatgaaggtttaccggagattaaagccgaggaggaagaagtggctacgaacgaaaccggccacctgttgcaaccgccgggtatcaatccgagcttttacgtgtatcagaccattagcccgcaggcataccgtccgcgtgacgagatcgtgctgccgccggagtacgcaggttacgtacgtgacccgaatgctccgattccgctgggtgtggtgcgtaaccgttgcgatatccaaaaagggtgcccgggctacatcccggaatggcagcgtgaagcaggtaccgcgatcagcccgaaaacaggcaaggccgtaactgtgccgggcctgtctccgaagaaaaacaaacgtatgcgtaggtactggcgctctgagaaggagaaagctcaagatgcgctgttggttaccgtacgcattggtaccgattgggttgtcatcgacgtgcgcggtctgttgcgcaacgcccgttggcgcaccatcgctcccaaggacatcagcctgaatgcgctcctcgatttgtttaccggtgatccggtgatcgatgtgcgtcgcaatattgttacctttacgtacaccctcgacgcctgtggtacctacgcccgtaaatggaccttgaaaggcaaacagacgaaagcgaccctggacaaactgacggccactcagacggttgcgttggttgcgattgcgcttggtcagaccaacccgatttccgctggcattagccgtgttacacaagaaaatggtgcactgcaatgcgaaccgctggaccggttcaccctgccggatgacctgttgaaggacatcagcgcatatcgcattgcttgggacagaaacgaagaggaactacgcgctcgcagcgtcgaggccctgccggaggcgcaacaagcggaggtccgtgcgctggacggggtgtccaaagaaaccgcgcgtacccaactgtgcgcagatttcggcctggacccgaaacgtctgccgtgggataaaatgagctccaatactacctttatcagcgaagccttgctgtctaacagcgtgagccgtgatcaggtttttttcactccggctccgaagaagggtgctaagaaaaaagcgcctgttgaagttatgcgtaaagaccgcacttgggcacgtgcgtacaagccgcgtctgagcgtcgaagctcagaaactgaagaacgaagccctgtgggcacttaagcgcacctccccggagtatttgaagctgagccgtcgcaaagaggagctgtgtcgtcgtagcatcaactatgttatcgaaaagacccgtcgccgtacgcaatgtcagattgtgatcccggtgatcgaggatctgaatgttcgcttctttcatggttctggtaagagactgcccggttgggataatttcttcaccgctaaaaaagagaaccgttggttcatccagggcctgcacaaagcgtttagcgatctgcgtacgcaccgttccttctatgtttttgaagttcgtccggagcgcacctctattacctgtccaaagtgcggtcattgcgaggtgggtaatcgtgacggtgaggcgtttcagtgcctgagctgcggcaagacgtgcaacgcagatcttgacgtcgcgacccataatctgacccaagtggcgttgaccggtaagaccatgccgaagcgcgaggagccgcgtgacgctcagggtacagcgccagcgcgcaagactaagaaggcgagcaaatcaaaggcgccaccggcggagcgcgaagaccagaccccggcgcaagagccaagccagaccagttctggaggtagcgggggtagcggtggtagcactaacttaagtgatattatcgagaaagaaaccgggaaacagctggtgatccaagaaagcattctgatgctgcccgaagaagttgaagaagtgattggtaacaagcctgagtcggacatcttagtgcacaccgcgtacgatgaaagcacggatgagaatgtgatgctgctcacctctgatgcccccgaatacaagccgtgggctctggtgatccaggactctaacggagaaaacaaaattaagatgctctctggcggctccatggcgcctaaaaaaaaacgcaaagtggggattcacggggttccggcgtcaggt

SEQ ID NO: 90 - DNA, 4026 bp, synthetic construct
  source 1..4026: mol_type = other DNA
atgtcaaaactagaaaagtttacaaattgttactccctgtcgaaaaccctgagattcaaagcgatcccggtgggtaaaactcaagagaacattgataacaaacgtctgctggttgaggacgaaaagcgcgcggaggattacaaaggcgttaaaaagctgttggaccgttattacctgtcctttattaatgatgttctgcacagcattaaattaaagaacctaaacaattacatctccctcttccgtaaaaagacccgcaccgaaaaggaaaataaagaactggagaaccttgagattaatctgcgtaaagaaatcgctaaagcgtttaaaggcaacgagggttataaatccttatttaaaaaagacatcatcgaaacgattctcccagaattcctggacgacaaagacgagatcgcgctggttaattccttcaacggctttaccaccgcattcaccggcttttttgataaccgcgaaaacatgttctctgaagaggccaaaagcacctcaattgcgtttcgctgcattaacgaaaatctgacgcgttatatctctaacatggatatcttcgagaaggttgacgctatctttgataaacatgaagtccaggagatcaaggagaagatcctgaacagcgactacgacgtggaggatttcttcgagggtgagttcttcaattttgttttgacccaagaaggcatcgatgtctacaatgcgataattggtggttttgttaccgagagcggggagaaaattaagggtctgaacgaatatatcaacttgtacaaccagaaaaccaaacaaaaactgccgaagtttaagccgctgtacaagcaagtcttgtcggatcgtgaatcgctgagcttttacggcgaaggctacaccagcgatgaggaggtgttggaggtgttccgcaatacgttgaataagaactctgaaattttttccagcattaagaagctggaaaaactattcaagaacttcgacgagtatagcagcgcaggtatctttgtgaaaaacggtccggctattagcaccatctcgaaagacattttcggcgaatggaatgttattcgtgataagtggaatgccgaatacgacgacattcatctgaagaaaaaggcggttgtaactgaaaagtacgaggacgatcgtcgtaagtcgttcaaaaagatcggctccttctctctggagcaactgcaagagtacgccgatgccgatttgtcagtcgtggagaagctgaaagagatcatcattcaaaaagttgacgaaatctataaggtctatggtagcagtgaaaaactgtttgacgcggacttcgtgctggagaaaagcctgaagaaaaatgacgccgtggtggcaatcatgaaagatcttctggatagcgtcaagtctttcgaaaactacatcaaagcgtttttcggtgaaggcaaggaaacgaatcgtgacgagagcttctatggtgatttcgtcctagcctatgatatcctgcttaaggtggatcacatctatgatgccatccgcaactatgttacccagaaaccgtactcaaaggacaagttcaagttatacttccagaatccgcagtttatgggtggctgggataaagataaggaaaccgactatcgcgccaccattttacgttacggtagtaagtactatctggcaatcatggataaaaaatatgcgaaatgcctgcaaaagatcgacaaggacgatgttaacggcaattatgagaagatcaactacaaactgctgccgggcccaaataagatgctgccgaaggtgtttttcagcaaaaaatggatggcgtattacaatccgagcgaggacatccaaaagatctacaaaaacggtacgtttaagaagggtgatatgttcaacctcaacgattgtcataaacttattgacttcttcaaggatagcatatctcgttatccgaagtggagcaacgcgtatgactttaactttagcgaaaccgagaaatacaaagatatcgcgggtttctatcgtgaagttgaagaacagggttacaaagtgtccttcgagtccgcgagcaagaaggaggtagacaagctggtcgaggagggtaagttgtacatgtttcagatttacaataaggacttttccgacaagtcccatggtacaccgaatttgcacaccatgtatttcaagcttctgttcgacgagaacaaccacggccagatccgtctgagcggtggtgcagagctgttcatgcgtagagctagcctgaagaaagaagagttggtcgttcatccggctaattcgccgatcgcgaacaaaaatccagataaccctaaaaagactacgaccctgagctatgatgtgtataaagacaaacgctttagcgaagaccagtatgagttacacattccgatcgctatcaacaagtgcccgaagaatattttcaagatcaacaccgaagtgcgtgttcttttgaagcacgatgacaacccgtatgtgattggtattgctcgtggtgaacgtaatctgctgtacattgtggttgtggacggcaaaggaaacatcgtggaacagtacagcctgaacgaaatcattaacaatttcaacggcattcgcattaagacggattaccattctttgctggacaaaaaggagaaagagcgtttcgaggcccgtcagaactggacctccatcgagaacattaaggagctgaaggcgggttacatcagccaggtggtgcacaaaatctgcgaactggtggagaagtacgacgcggtgattgctttggcggatttgaactccggttttaagaacagccgcgttaaagtggaaaagcaggtttatcagaaattcgaaaaaatgctgattgataaattgaattacatggttgacaaaaagtctaacccgtgtgcaaccggcggtgcgctgaagggctaccaaatcaccaataagtttgaaagctttaagagcatgagcacgcaaaatggcttcattttttacattccggcgtggctgaccagcaagatcgatccgtcaaccggctttgttaatttactgaagacgaagtatacctctattgccgactctaaaaaatttattagttccttcgatagaatcatgtatgttccggaagaggacctgttcgaatttgcactggactacaagaactttagccgtaccgatgctgattatataaagaaatggaaactctacagctatggcaaccgcatccgtatctttcgcaacccgaaaaaaaacaacgtgttcgactgggaagaggtatgtctgacttcagcgtacaaggagcttttcaataaatatggtatcaactaccagcagggtgatatccgtgcactgctttgcgagcaaagcgataaagcgttctattcaagcttcatggcgctgatgagcctgatgctgcaaatgcgcaacagcatcaccggtcgtaccgatgttgcatttctgatttctccagtaaaaaacagcgatggtatcttttacgacagccgtaattacgaggcccaagaaaatgcgattttgccgaagaacgctgacgcgaatggtgcatacaacattgcaaggaaagttctgtgggcaatcggccaatttaaaaaagcggaagatgagaagctggacaaagtgaaaattgcgattagcaacaaggagtggctggagtatgcgcagacctccgttaaatcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggt

SEQ ID NO: 91 - DNA, 43 bp, synthetic construct
  source 1..43: mol_type = other DNA
taatttctactcttgtagatgtcagcaacgacaggcagctggg

SEQ ID NO: 92 - DNA, 48 bp, synthetic construct
  source 1..48: mol_type = other DNA
caacgattgcccctcacgaggggacgtcagcaacgacaggcagctggg

SEQ ID NO: 93 - DNA, 102 bp, synthetic construct
  source 1..102: mol_type = other DNA
gtcagcaacgacaggcagctgttttagagctagaaatagcaagttaaaataaggctagtccgttatcaacttgaaaaagtggcaccgagtcggtgctttttt

SEQ ID NO: 94 - DNA, 58 bp, synthetic construct
  source 1..58: mol_type = other DNA
gtcaaaagacctttttaatttctactcttgtagatgtcagcaacgacaggcagctggg

SEQ ID NO: 95 - DNA, 42 bp, synthetic construct
  source 1..42: mol_type = other DNA
aatttctactgttgtagatgtcagcaacgacaggcagctggg

SEQ ID NO: 96 - DNA, 43 bp, synthetic construct
  source 1..43: mol_type = other DNA
aatttctactaagtgtagatgtcagcaacgacaggcagctggg

SEQ ID NO: 97 - DNA, 43 bp, synthetic construct
  source 1..43: mol_type = other DNA
aatttctactgtttgtagatgtcagcaacgacaggcagctggg

SEQ ID NO: 98 - DNA, 1014 bp, synthetic construct
  source 1..1014: mol_type = other DNA
tctggaagtgagacacccgggacttcagaatccgcgaccccggagtctatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggctccggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggcatggacgagctgtacaagtcaggtgctgcagcggccgaattcaagcgtgaagaacaagcaaggaaagctaaggtgaacaatgagaaaaagacggaaatagtgaaaccagagagttgtagcaatgaaggagatgtcaaggatctgaaaagaaaggactctgaggatggaaacgagggtgaggaagaagaagcttcttcgaaaccgaaaaagccaaaagttgctctttctcatcttcaggacattgacgacacagaagctgatcaagaagaagagtaa

SEQ ID NO: 99 - DNA, 466 bp, synthetic construct
  source 1..466: mol_type = other DNA
tggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagctta

SEQ ID NO: 100 - DNA, 857 bp, synthetic construct
  source 1..857: mol_type = other DNA
agattagccttttcaatttcagaaagaatgctaacccacagatggttagagaggcttacgcagcaagtctcatcaagacgatctacccgagcaataatctacaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagtagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaaaacgttccaaccacgtctacaaagcaagtggattgatgtgatatctccactgacgtaagggatgacgcacaatcccactatccttcgcaagacccttcctctatataaggaagttcatttcatttggagagaacacgggggactctagaggatccaaca

SEQ ID NO: 101 - DNA, 6897 bp, synthetic construct
  source 1..6897: mol_type = other DNA
tggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttaatgggaagtcatcaccaccaccaccattcaggtgagaacctgtacttccagagcatggctccgaaaaagaaacgtaaggtgggtatccacggcgttccggcgatggaagcaagtccagcttcaggacccaggcacctgatggacccgcatattttcacctctaattttaacaacggtattggccgtcacaagacgtacctgtgttatgaagtggaacgtcttgacaacggcacctccgtgaaaatggaccagcatcgtggtttcttgcataatcaagctaaaaacctgctgtgcggtttctacggccgtcacgcggaactgcgttttttggacctggttccgagcctgcaactggacccggcacagatttatcgtgttacttggttcatcagctggtcgccgtgttttagctggggttgcgccggcgaggtacgcgcgtttttgcaagagaacacccacgtgcgtttgcgcatcttcgccgctcgtatctacgattatgacccactgtataaagaagcgctccagatgctgcgcgatgcaggcgcgcaggtttccatcatgacctacgacgagttcaagcactgctgggatacctttgtcgatcatcagggctgcccgttccagccgtgggatggtctggatgagcacagccaggcgttaagcggtcgcctcagagcgattctgcaaaaccaaggtaattctggaagtgagacacccgggacttcagaatccgcgaccccggagtctggaatggataaaaagtattcaataggactagctattggcaccaacagcgttggttgggcagttatcaccgatgaatataaggtcccgagcaagaagtttaaggtcctgggcaacaccgatcgccacagcataaaaaagaaccttattggtgcgctgctgttcgattctggtgagactgccgaagcgacccgtctgaagagaaccgcgcgtcgtagatacacccggcgcaagaaccgtatctgctatctgcaagaaattttcagcaacgagatggctaaggtcgacgactccttctttcatcgcctggaggaaagctttctggttgaggaagacaaaaagcacgagcgtcatccgattttcggtaacattgttgatgaagttgcctaccacgaaaaataccccacaatttatcatctgcgtaaaaaactggtagactccaccgataaggcggatttacgccttatctatttggcgctggcgcatatgattaagttccgcggtcattttttgatcgagggcgacctgaatccggataacagcgacgttgacaaactgtttatccagctggtgcagacctataaccagttgttcgaagagaacccgattaatgcgtctggcgtggacgcgaaggcgatcctgagcgctcgtctgtccaagtcacgccgcctggagaacttgatcgcgcaactgccgggtgagaagaaaaacggcttgttcggcaatctgattgctctgtccctaggtttgaccccgaatttcaaatctaattttgatctggcggaagatgcgaaattacagttgagcaaggacacttacgatgacgatctggacaacctgctcgctcagatcggcgatcagtatgccgatcttttcctggccgcgaagaacctgagcgatgccatcttgctatcggacatcttacgtgttaacaccgaaatcaccaaagctccgctaagcgcgtccatgattaaacgttacgatgaacaccaccaggatctgacgctgctgaaagcgttggtgcgccagcaactgccggaaaaatacaaggaaattttcttcgaccaaagcaagaacggttatgcaggctatatcgacggtggtgcgtcgcaagaagaattttataaattcatcaaaccgattttggagaagatggatggtaccgaggagctgctggtaaaattgaatcgggaagacctcttgcgaaagcagcgtactttcgataatggtagcatcccgcaccagattcatctgggcgaacttcacgcaatcctacgtcgtcaagaggatttctacccgtttctgaaggataaccgtgaaaagatcgagaagattctgacctttcgtattccgtattatgtgggtccgctggcgagaggcaacagccgttttgcgtggatgacccgcaagtcggaagagactatcacgccgtggaatttcgaggaggtcgtggacaagggtgcatcagcgcaatcttttattgaacgtatgaccaattttgataaaaaccttccaaatgaaaaagtgttgccgaagcacagcttgctgtacgagtatttcaccgtttacaatgaactgacgaaagtcaagtacgttaccgaaggtatgcgtaagccagcctttctgtctggggagcaaaaaaaagccattgtggatctgctgttcaagaccaatcgcaaggttaccgtcaagcaactgaaagaggattacttcaaaaaaatcgaatgttttgactccgtggaaatcagcggcgttgaagaccgctttaatgcatctctgggcacctaccacgatctactcaagatcatcaaggataaggacttcctggacaacgaagaaaatgaggatatcttggaagatattgtgctgactttaacgttgttcgaggatcgtgagatgattgaagaacgcctcaaaacgtacgcacatctgtttgacgataaggtgatgaaacagctaaagcgccgtcgttacaccggttggggtcgtctgtctagaaagctgattaacggcattcgtgacaaacaatctggcaagaccatcctggacttcctcaaatccgacggcttcgcaaatcgtaatttcatgcaactgatccatgatgatagcctgacctttaaagaagatattcagaaggctcaagtgtccggccagggtgactcgctgcatgagcatatcgcgaatctggctggctcccctgcgataaaaaaaggtattctccaaacggtgaaagttgtggatgaactggttaaagtcatgggtcgtcataaaccggaaaacatcgtgattgagatggcacgtgagaaccagaccacccagaaaggtcaaaaaaacagtcgtgagcgtatgaaacgtatcgaagaggggattaaggagttaggcagccagattctgaaggagcacccggttgagaacacccagctgcaaaacgagaagctctacctgtattacctgcaaaacggccgtgacatgtatgttgaccaggagctggacatcaatcgtctcagcgactacgacgtggatgctatcgtgccgcagagctttctaaaagatgattccatcgacaataaagtgctcacccgtagcgacaagaaccgtggtaaaagcgacaacgtgccgagcgaggaagtagttaagaaaatgaaaaactactggcgtcagctattgaacgccaagcttatcacccagcgtaagtttgacaacttgacgaaggcagagcgtggcggcctgagcgagctagacaaggcgggttttattaaacgccagctggtggaaactcgccagattaccaaacacgttgcgcaaatcttggactcacgcatgaatacgaagtacgacgaaaacgataaattgatccgcgaggtgaaggtcataaccctgaaatctaaactggttagcgatttccggaaggactttcaattttataaagtccgggagattaacaactatcaccatgcacacgacgcttacttaaatgcggttgttggcacggccctgatcaagaagtacccgaaactggaaagcgagtttgtttatggtgactacaaggtgtatgacgttcgcaagatgattgcaaaatccgaacaagagattggtaaggcgacggctaagtattttttctacagcaatatcatgaacttcttcaagactgaaatcaccctggcgaacggtgaaattcgtaaacgtccgttgatcgaaaccaacggtgaaaccggtgaaatcgtttgggataaaggtagggattttgcgaccgttcgtaaggtcctgagcatgccgcaggttaacatcgtgaaaaagaccgaagtgcagaccggcggtttctccaaggagagtatcttaccgaagcgcaacagtgacaagctgatcgcgcgtaaaaaagactgggatccgaaaaaatacggcggctttgactccccgaccgtcgcgtacagcgtactggtcgtggctaaagtggagaaaggcaaatcgaaaaagctgaaatccgttaaagagttgctgggtattacgattatggaacgttcgagcttcgagaaaaatccgatcgacttcctggaggccaaaggctacaaggaagttaaaaaagacctgatcattaaactgccgaagtacagcctgttcgagctcgagaacggtcgcaaacgcatgctggcgagcgcgggtgagctccaaaaaggcaacgaattggctctgccgagcaagtacgtgaacttcctgtatctggcctcccattatgaaaaactgaagggtagcccggaagataacgaacaaaagcagctgttcgtggaacaacataaacactatctggacgagattattgagcaaatcagcgagttcagcaaacgcgtgattctggcggatgctaatctggacaaggttctttcagcctataataaacaccgtgataagccgatccgtgaacaggcagaaaatattattcacctgttcaccctgacgaacctgggtgcgccagcagcgtttaaatacttcgacacaacgatcgaccggaagcgttataccagcaccaaagaggttcttgacgccaccttgattcaccaaagcatcacgggtctgtatgaaacccgtattgacttgagccagttgggcggagattcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttcaggttctggaagtgagacacccgggacttcagaatccgcgaccccggagtctatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggctccggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggcatggacgagctgtacaagtcaggtgctgcagcggccgaattcaagcgtgaagaacaagcaaggaaagctaaggtgaacaatgagaaaaagacggaaatagtgaaaccagagagttgtagcaatgaaggagatgtcaaggatctgaaaagaaaggactctgaggatggaaacgagggtgaggaagaagaagcttcttcgaaaccgaaaaagccaaaagttgctctttctcatcttcaggacattgacgacacagaagctgatcaagaagaagagtaagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgct

SEQ ID NO: 102 - DNA, 7288 bp, synthetic construct
  source 1..7288: mol_type = other DNA
agattagccttttcaatttcagaaagaatgctaacccacagatggttagagaggcttacgcagcaagtctcatcaagacgatctacccgagcaataatctacaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagtagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaaaacgttccaaccacgtctacaaagcaagtggattgatgtgatatctccactgacgtaagggatgacgcacaatcccactatccttcgcaagacccttcctctatataaggaagttcatttcatttggagagaacacgggggactctagaggatccaacaatgggaagtcatcaccaccaccaccattcaggtgagaacctgtacttccagagcatggctccgaaaaagaaacgtaaggtgggtatccacggcgttccggcgatggaagcaagtccagcttcaggacccaggcacctgatggacccgcatattttcacctctaattttaacaacggtattggccgtcacaagacgtacctgtgttatgaagtggaacgtcttgacaacggcacctccgtgaaaatggaccagcatcgtggtttcttgcataatcaagctaaaaacctgctgtgcggtttctacggccgtcacgcggaactgcgttttttggacctggttccgagcctgcaactggacccggcacagatttatcgtgttacttggttcatcagctggtcgccgtgttttagctggggttgcgccggcgaggtacgcgcgtttttgcaagagaacacccacgtgcgtttgcgcatcttcgccgctcgtatctacgattatgacccactgtataaagaagcgctccagatgctgcgcgatgcaggcgcgcaggtttccatcatgacctacgacgagttcaagcactgctgggatacctttgtcgatcatcagggctgcccgttccagccgtgggatggtctggatgagcacagccaggcgttaagcggtcgcctcagagcgattctgcaaaaccaaggtaattctggaagtgagacacccgggacttcagaatccgcgaccccggagtctggaatggataaaaagtattcaataggactagctattggcaccaacagcgttggttgggcagttatcaccgatgaatataaggtcccgagcaagaagtttaaggtcctgggcaacaccgatcgccacagcataaaaaagaaccttattggtgcgctgctgttcgattctggtgagactgccgaagcgacccgtctgaagagaaccgcgcgtcgtagatacacccggcgcaagaaccgtatctgctatctgcaagaaattttcagcaacgagatggctaaggtcgacgactccttctttcatcgcctggaggaaagctttctggttgaggaagacaaaaagcacgagcgtcatccgattttcggtaacattgttgatgaagttgcctaccacgaaaaataccccacaatttatcatctgcgtaaaaaactggtagactccaccgataaggcggatttacgccttatctatttggcgctggcgcatatgattaagttccgcggtcattttttgatcgagggcgacctgaatccggataacagcgacgttgacaaactgtttatccagctggtgcagacctataaccagttgttcgaagagaacccgattaatgcgtctggcgtggacgcgaaggcgatcctgagcgctcgtctgtccaagtcacgccgcctggagaacttgatcgcgcaactgccgggtgagaagaaaaacggcttgttcggcaatctgattgctctgtccctaggtttgaccccgaatttcaaatctaattttgatctggcggaagatgcgaaattacagttgagcaaggacacttacgatgacgatctggacaacctgctcgctcagatcggcgatcagtatgccgatcttttcctggccgcgaagaacctgagcgatgccatcttgctatcggacatcttacgtgttaacaccgaaatcaccaaagctccgctaagcgcgtccatgattaaacgttacgatgaacaccaccaggatctgacgctgctgaaagcgttggtgcgccagcaactgccggaaaaatacaaggaaattttcttcgaccaaagcaagaacggttatgcaggctatatcgacggtggtgcgtcgcaagaagaattttataaattcatcaaaccgattttggagaagatggatggtaccgaggagctgctggtaaaattgaatcgggaagacctcttgcgaaagcagcgtactttcgataatggtagcatcccgcaccagattcatctgggcgaacttcacgcaatcctacgtcgtcaagaggatttctacccgtttctgaaggataaccgtgaaaagatcgagaagattctgacctttcgtattccgtattatgtgggtccgctggcgagaggcaacagccgttttgcgtggatgacccgcaagtcggaagagactatcacgccgtggaatttcgaggaggtcgtggacaagggtgcatcagcgcaatcttttattgaacgtatgaccaattttgataaaaaccttccaaatgaaaaagtgttgccgaagcacagcttgctgtacgagtatttcaccgtttacaatgaactgacgaaagtcaagtacgttaccgaaggtatgcgtaagccagcctttctgtctggggagcaaaaaaaagccattgtggatctgctgttcaagaccaatcgcaaggttaccgtcaagcaactgaaagaggattacttcaaaaaaatcgaatgttttgactccgtggaaatcagcggcgttgaagaccgctttaatgcatctctgggcacctaccacgatctactcaagatcatcaaggataaggacttcctggacaacgaagaaaatgaggatatcttggaagatattgtgctgactttaacgttgttcgaggatcgtgagatgattgaagaacgcctcaaaacgtacgcacatctgtttgacgataaggtgatgaaacagctaaagcgccgtcgttacaccggttggggtcgtctgtctagaaagctgattaacggcattcgtgacaaacaatctggcaagaccatcctggacttcctcaaatccgacggcttcgcaaatcgtaatttcatgcaactgatccatgatgatagcctgacctttaaagaagatattcagaaggctcaagtgtccggccagggtgactcgctgcatgagcatatcgcgaatctggctggctcccctgcgataaaaaaaggtattctccaaacggtgaaagttgtggatgaactggttaaagtcatgggtcgtcataaaccggaaaacatcgtgattgagatggcacgtgagaaccagaccacccagaaaggtcaaaaaaacagtcgtgagcgtatgaaacgtatcgaagaggggattaaggagttaggcagccagattctgaaggagcacccggttgagaacacccagctgcaaaacgagaagctctacctgtattacctgcaaaacggccgtgacatgtatgttgaccaggagctggacatcaatcgtctcagcgactacgacgtggatgctatcgtgccgcagagctttctaaaagatgattccatcgacaataaagtgctcacccgtagcgacaagaaccgtggtaaaagcgacaacgtgccgagcgaggaagtagttaagaaaatgaaaaactactggcgtcagctattgaacgccaagcttatcacccagcgtaagtttgacaacttgacgaaggcagagcgtggcggcctgagcgagctagacaaggcgggttttattaaacgccagctggtggaaactcgccagattaccaaacacgttgcgcaaatcttggactcacgcatgaatacgaagtacgacgaaaacgataaattgatccgcgaggtgaaggtcataaccctgaaatctaaactggttagcgatttccggaaggactttcaattttataaagtccgggagattaacaactatcaccatgcacacgacgcttacttaaatgcggttgttggcacggccctgatcaagaagtacccgaaactggaaagcgagtttgtttatggtgactacaaggtgtatgacgttcgcaagatgattgcaaaatccgaacaagagattggtaaggcgacggctaagtattttttctacagcaatatcatgaacttcttcaagactgaaatcaccctggcgaacggtgaaattcgtaaacgtccgttgatcgaaaccaacggtgaaaccggtgaaatcgtttgggataaaggtagggattttgcgaccgttcgtaaggtcctgagcatgccgcaggttaacatcgtgaaaaagaccgaagtgcagaccggcggtttctccaaggagagtatcttaccgaagcgcaacagtgacaagctgatcgcgcgtaaaaaagactgggatccgaaaaaatacggcggctttgactccccgaccgtcgcgtacagcgtactggtcgtggctaaagtggagaaaggcaaatcgaaaaagctgaaatccgttaaagagttgctgggtattacgattatggaacgttcgagcttcgagaaaaatccgatcgacttcctggaggccaaaggctacaaggaagttaaaaaagacctgatcattaaactgccgaagtacagcctgttcgagctcgagaacggtcgcaaacgcatgctggcgagcgcgggtgagctccaaaaaggcaacgaattggctctgccgagcaagtacgtgaacttcctgtatctggcctcccattatgaaaaactgaagggtagcccggaagataacgaacaaaagcagctgttcgtggaacaacataaacactatctggacgagattattgagcaaatcagcgagttcagcaaacgcgtgattctggcggatgctaatctggacaaggttctttcagcctataataaacaccgtgataagccgatccgtgaacaggcagaaaatattattcacctgttcaccctgacgaacctgggtgcgccagcagcgtttaaatacttcgacacaacgatcgaccggaagcgttataccagcaccaaagaggttcttgacgccaccttgattcaccaaagcatcacgggtctgtatgaaacccgtattgacttgagccagttgggcggagattcagggggaagtggcggaagcggtgggtctacgaacctgagcgacattattgaaaaagaaactggtaaacagttggtgatccaagaatccattctgatgttaccggaggaagtggaagaggtgatcggcaacaagccggagtccgacatcctggttcacaccgcgtatgatgagtcgaccgatgagaatgttatgctgctgaccagcgacgccccagagtacaaaccgtgggcgctcgtcatccaggatagcaatggcgaaaacaagatcaagatgttgtctggcggtagcatggctccgaaaaagaagcgtaaagtaggtattcatggtgttccggcatcaggttcaggttctggaagtgagacacccgggacttcagaatccgcgaccccggagtctatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggctccggataccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggcatggacgagctgtacaagtcaggtgctgcagcggccgaattcaagcgtgaagaacaagcaaggaaagctaaggtgaacaatgagaaaaagacggaaatagtgaaaccagagagttgtagcaatgaaggagatgtcaaggatctgaaaagaaaggactctgaggatggaaacgagggtgaggaagaagaagcttcttcgaaaccgaaaaagccaaaagttgctctttctcatcttcaggacattgacgacacagaagctgatcaagaagaagagtaagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgct